SEQUENCE LISTING (70 sequences)

SEQ ID NO: 1 - protein, 133 aa, Homo sapiens [sequence omitted]

SEQ ID NO: 2 - protein, 114 aa, Homo sapiens
  Human IL-15 (mature; UniProt P40933)
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - protein, 138 aa, Homo sapiens
  Human IL-21 (mature; UniProt Q9HBE4)
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 4 - protein, 133 aa, Artificial Sequence [sequence omitted]

SEQ ID NO: 5 - protein, 387 aa, Artificial Sequence
  Membrane-bound IL-15 (mbIL-15)
MLLLVTSLLLCELPHPAFLLIPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFF

SEQ ID NO: 6 - protein, 395 aa, Artificial Sequence
  Membrane-bound IL-21 (mbIL-21)
MLLLVTSLLLCELPHPAFLLIPQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKVSTLSFIESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFF

SEQ ID NO: 7 - protein, 204 aa, Artificial Sequence
  Membrane-bound IL-15 (mbIL-15)
MALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 8 - protein, 204 aa, Artificial Sequence
  Membrane-bound IL-15 (mbIL-15)
MALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVSPFTA

SEQ ID NO: 9 - DNA, 489 bp, Artificial Sequence
  IL-15 nucleotide
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 10 - protein, 67 aa, Artificial Sequence
  CD28 transmembrane domain
IEVMYPPPYLDNEKSNGTIIHVKGKHLCPSPLFPGPSKPFWVLVVVGGVLACYSLLVTVAFIIFWVR

SEQ ID NO: 11 - protein, 69 aa, Artificial Sequence
  CD8 transmembrane domain
TTTPAPRPPTPAPTIASQPLSLRPEACRPAAGGAVHTRGLDFACDIYIWAPLAGTCGVLLLSLVITLYC

SEQ ID NO: 12 - protein, 21 aa, Artificial Sequence
  CD8alpha signal peptide
MALPVTALLLPLALLLHAARP

SEQ ID NO: 13 - protein, 22 aa, Artificial Sequence
  GM-CSFRa signal peptide
MLLLVTSLLLCELPHPAFLLIP

SEQ ID NO: 14 - protein, 20 aa, Artificial Sequence
  IgK signal peptide
MRLPAQLLGLLMLWVPGSSG

SEQ ID NO: 15 - protein, 22 aa, Artificial Sequence
  CD4 transmembrane domain
MALIVLGGVAGLLLFIGLGIFF

SEQ ID NO: 16 - RNA, 20 nt, Artificial Sequence
  Cpfl crRNA scaffold sequence
uaauuucuacucuuguagau

SEQ ID NO: 17 - DNA, 21 bp, Artificial Sequence
  primer
atatttacagaatggcacagg

SEQ ID NO: 18 - DNA, 20 bp, Artificial Sequence
  Primer
gacttggtacccaggttgaa

SEQ ID NO: 19 - DNA, 32 bp, Artificial Sequence
  Primer
atcagattcgatcctacttctgcagggggcat

SEQ ID NO: 20 - DNA, 32 bp, Artificial Sequence
  Primer
acgtgctgagcttgagtgatggtgatgttcac

SEQ ID NO: 21 - DNA, 24 bp, Artificial Sequence
  Primer
cccaactcaacttcccagtgtgat

SEQ ID NO: 22 - DNA, 30 bp, Artificial Sequence
  Primer
gaaatctaccttttcctctaatagggcaat

SEQ ID NO: 23 - DNA, 29 bp, Artificial Sequence
  Primer
gaaatctaccttttcctctaatagggcaa

SEQ ID NO: 24 - DNA, 28 bp, Artificial Sequence
  Primer
gaaatctaccttttcctctaatagggca

SEQ ID NO: 25 - DNA, 21 bp, Artificial Sequence
  Primer
ccaaaagccacactcaaagac

SEQ ID NO: 26 - DNA, 21 bp, Artificial Sequence
  Primer
acccaggtggaaagaatgatg

SEQ ID NO: 27 - DNA, 25 bp, Artificial Sequence
  Primer
aacatcaccatcactcaaggtttgg

SEQ ID NO: 28 - DNA, 25 bp, Artificial Sequence
  Primer
ctgaagacacatttttactcccaaa

SEQ ID NO: 29 - DNA, 22 bp, Artificial Sequence
  Primer
tccaaaagccacactcaaagac

SEQ ID NO: 30 - DNA, 25 bp, Artificial Sequence
  Primer
ctgaagacacatttttactcccaac

SEQ ID NO: 31 - protein, 238 aa, Artificial Sequence
  CD16 (F158)
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 32 - protein, 16 aa, Artificial Sequence
  Signal peptide
MWQLLLPTALLLLVSA

SEQ ID NO: 33 - protein, 238 aa, Artificial Sequence
  CD16 (V158 or 158V+)
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 34 - protein, 86 aa, Artificial Sequence
  FcRgamma
MIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 35 - protein, 105 aa, Artificial Sequence
  Anti-CD20 (Leu16) VL
IVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 36 - protein, 117 aa, Artificial Sequence
  Anti-CD20 (Leu16) VH
GAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSLD

SEQ ID NO: 37 - protein, 247 aa, Artificial Sequence
  Anti-CD20 (Leu16) scFv
IVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSLD

SEQ ID NO: 38 - protein, 230 aa, Artificial Sequence
  IgG4 Fc spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKM

SEQ ID NO: 39 - protein, 27 aa, Artificial Sequence
  CD28 transmembrane domain
FWVLVVVGGVLACYSLLVTVAFIIFWV

SEQ ID NO: 40 - protein, 41 aa, Artificial Sequence
  CD28 costimulatory signaling domain
RSKRSRGGHSDYMNMTPRRPGPTRKHYQPYAPPRDFAAYRS

SEQ ID NO: 41 - protein, 112 aa, Artificial Sequence
  CD3 zeta signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 657 aa, Artificial Sequence
  Anti-CD20 CAR
IVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSLDESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 21 aa, Artificial Sequence
  IgK Signal peptide
METDTLLLWVLLLWVPGSTGD

SEQ ID NO: 44 - protein, 9 aa, Artificial Sequence
  HA tag
YPYDVPDYA

SEQ ID NO: 45 - DNA, 2064 bp, Artificial Sequence
  Anti-CD20 CAR
atggagacagacacactcctgctatgggtgctgctgctctgggttccaggttccacaggtgactacccatacgatgttccagattacgctattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcactcgacgaatctaagtacggaccgccctgccccccttgccctgcccccgagttcctgggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttcaacagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaagatgttctgggtgctggtggtggtgggcggggtgctggcctgctacagcctgctggtgacagtggccttcatcatcttttgggtgcggagcaagcggagcagaggcggccacagcgactacatgaacatgacccccagacggcctggccccacccggaagcactaccagccctacgccccacccagggactttgccgcctacagaagccgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtga

SEQ ID NO: 46 - protein, 121 aa, Artificial Sequence
  Anti-CD38 VH
QVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSS

SEQ ID NO: 47 - protein, 122 aa, Artificial Sequence
  Anti-CD38 VH
EVQLVQSGAEVKKPGESLKISCKGSGYSFSNYWIGWVRQMPGKGLEWMGIIYPHDSDARYSPSFQGQVTFSADKSISTAYLQWSSLKASDTAMYYCARHVGWGSRYWYFDLWGRGTLVTVSS

SEQ ID NO: 48 - protein, 107 aa, Artificial Sequence
  Anti-CD38 VL
EIVLTQSPDFQSVTPKEKVTITCRASQSIGSSLHWYQQKPDQSPKLLIKYASQSFSGVPSRFSGSGSGTDFTLTINSLEAEDAATYYCHQSSSLPYTFGQGTKLEIK

SEQ ID NO: 49 - protein, 107 aa, Artificial Sequence
  Anti-CD38 VL
DIQMTQSPSSLSASVGDRVTITCRASQGISNYLAWFQQKPGKAPKSLIYAASSLQSGVPSRFSGSGSGTDFTLTISSLQPEDFATYYCQQYNSYPYTFGQGTKLEIK

SEQ ID NO: 50 - protein, 112 aa, Artificial Sequence
  CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 42 aa, Artificial Sequence
  4-1BB costimulatory domain
KRGRKKLLYIFKQPFMRPVQTTQEEDGCSCRFPEEEEGGCEL

SEQ ID NO: 52 - protein, 41 aa, Artificial Sequence
  CD28 costimulatory domain
RSKRSRLLHSDYMNMTPRRPGPTRKHYQPYAPPRDFAAYRS

SEQ ID NO: 53 - protein, 107 aa, Artificial Sequence
  Anti-CD19 VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 54 - protein, 120 aa, Artificial Sequence
  Anti-CD19 VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 55 - protein, 18 aa, Artificial Sequence
  Whitlow linker
GSTSGSGKPGSGEGSTKG

SEQ ID NO: 56 - protein, 15 aa, Artificial Sequence
  GS linker
GGGGSGGGGSGGGGS

SEQ ID NO: 57 - protein, 245 aa, Artificial Sequence
  Anti-CD19 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 58 - protein, 242 aa, Artificial Sequence
  Anti-CD19 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 59 - protein, 12 aa, Artificial Sequence
  IgG4 hinge
ESKYGPPCPPCP

SEQ ID NO: 60 - protein, 25 aa, Artificial Sequence
  CD8 hinge
TTTPAPRPPTPAPTIASQPLSLRPE

SEQ ID NO: 61 - protein, 44 aa, Artificial Sequence
  CD8 transmembrane
ACRPAAGGAVHTRGLDFACDIYIWAPLAGTCGVLLLSLVITLYC

SEQ ID NO: 62 - protein, 111 aa, Artificial Sequence
  Anti-BCMA C11D5.3 VL
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 63 - protein, 117 aa, Artificial Sequence
  Anti-BCMA C11D5.3 VH
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 64 - protein, 246 aa, Artificial Sequence
  Anti-BCMA C11D5.3 scFv
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 65 - protein, 111 aa, Artificial Sequence
  Anti-BCMA C12A3.2 VL
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 66 - protein, 117 aa, Artificial Sequence
  Anti-BCMA C12A3.2 VH
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 67 - protein, 246 aa, Artificial Sequence
  Anti-BCMA C12A3.2 scFv
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 68 - protein, 110 aa, Artificial Sequence
  Anti-BCMA BCMA02 VH
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQG

SEQ ID NO: 69 - protein, 110 aa, Artificial Sequence
  Anti-BCMA BCMA02 VL
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEI

SEQ ID NO: 70 - protein, 246 aa, Artificial Sequence
  Anti-BCMA02 scFv
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

(19) United States
(12) Patent Application Publication
(10) Pub. No.: US 2024/0316102 A1
(43) Pub. Date: Sep. 26, 2024

(54) ENGINEERED NATURAL KILLER (NK) CELLS AND RELATED METHODS

(71) Applicant: Indapta Therapeutics, Inc., Houston, TX (US)

(72) Inventors: Guy DIPIERRO, Houston, TX (US); Austin BIGLEY, Houston, TX (US)

(73) Assignee: Indapta Therapeutics, Inc., Houston, TX (US)

(21) Appl. No.: 18/573,893

(22) PCT Filed: Jun. 30, 2022
(86) PCT No.: PCT/US2022/035884

§ 371 Date: Dec. 22, 2023

Related U.S. Application Data
(60) Provisional application No. 63/217,722, filed on Jul. 1, 2021.
(60) Provisional application No. 63/217,718, filed on Jul. 1, 2021.
(60) Provisional application No. 63/217,726, filed on Jul. 1, 2021.

(51) Int. Cl. A61K 35/17; A61K 39/00; A61P 35/00; C07K 14/54; C07K 14/55; C07K 14/725; C12N 5/0783; C12N 15/86

(52) U.S. Cl. CPC A61K 35/17; A61K 39/4613; A61K 39/4631; A61K 39/464424; A61P 35/00; C07K 14/5443; C07K 14/55; C07K 14/7051; C12N 5/0646; C12N 15/86; A61K 2239/13; A61K 2239/21; A61K 2239/48; C07K 2317/622; C07K 2319/02; C07K 2319/03; C12N 2501/2302; C12N 2501/2312; C12N 2501/2315; C12N 2501/2318; C12N 2501/2321; C12N 2501/2327; C12N 2510/00; C12N 2740/15043

(57) ABSTRACT

Provided herein are engineered Natural Killer (NK) cells deficient in expression of FcRγ chain (g-NK cells), and further comprising a recombinant chimeric antigen receptor (CAR) and compositions thereof. Also provided herein are compositions containing the engineered NK cells and methods of making and using the engineered NK cells.

### CROSS-REFERENCE TO RELATED APPLICATIONS

This application claims priority from U.S. provisional application No. 63/217,718, filed Jul. 1, 2021, entitled “ENGINEERED NATURAL KILLER (NK) CELLS AND RELATED METHODS,” U.S. provisional application No. U.S. 63/217,722, filed Jul. 1, 2021, entitled “ENGINEERED NATURAL KILLER (NK) CELLS AND RELATED METHODS,” and U.S. provisional application No. 63/217,726, filed Jul. 1, 2021, entitled “ENGINEERED NATURAL KILLER (NK) CELLS AND RELATED METHODS.” The contents of which are incorporated by reference in their entirety.

### INCORPORATION BY REFERENCE OF SEQUENCE LISTING

The present application is being filed with a Sequence Listing in electronic format. The Sequence Listing is provided as a file entitled 77603_2001040_SEQLISTING.txt, created on Jun. 30, 2022, which is 69,307 bytes in size. The information in electronic format of the Sequence Listing is incorporated by reference in its entirety.

### FIELD

The present disclosure provides engineered Natural Killer (NK) cells deficient in expression of FcRγ chain (g-NK cells), and further comprising a recombinant chimeric antigen receptor (CAR) and compositions thereof. The present disclosure also provides compositions containing the engineered NK cells and methods of making and using the engineered NK cells.

### BACKGROUND

Antibody-based therapy has become frequently used for treating cancers and other diseases. Responses to antibody therapy have typically focused on the direct inhibitory effects of these antibodies on the tumor cells (e.g. inhibition of growth factor receptors and the subsequent induction of apoptosis), but the in vivo effects of these antibodies may be more complex and may involve the host immune system. Natural killer (NK) cells are immune effector cells that mediate antibody-dependent cellular cytotoxicity when the Fc receptor (CD16; FcγRIII) binds to the Fc portion of antibodies bound to an antigen-bearing cell. NK cells, including specific specialized subsets thereof, can be used in therapeutic methods, including for improving responses to antibody therapy. Improved methods are needed for therapeutic uses involving NK cells. Provided herein are embodiments that meet such needs.

### SUMMARY

Described herein are engineered Natural Killer (NK) cells deficient in expression of FcRγ chain (g-NK cells), and further comprising a recombinant chimeric antigen receptor (CAR) and compositions thereof. Also described herein are compositions containing the engineered NK cells and methods of making and using the engineered NK cells.

In some aspects, provided herein, is an engineered Natural Killer (NK) cell that is deficient in expression of FcRγ chain (g-NK cells) and comprises a heterologous nucleic acid encoding a chimeric antigen receptor (CAR).

In some aspects, provided herein, is an engineered Natural Killer (NK) cell that is deficient in expression of FcRγ chain (g-NK cells), and comprises a heterologous nucleic acid encoding an immunomodulator.

In some aspects, provided herein, is an engineered Natural Killer (NK) cell that is deficient in expression of FcRγ chain (g-NK cells), and comprises a heterologous nucleic acid encoding a chimeric antigen receptor (CAR) and a heterologous nucleic acid encoding an immunomodulator.

In some embodiments, the CAR comprises 1) an antigen binding domain; 2) a flexible linker; 3) a transmembrane region; and 4) an intracellular signaling domain. In some embodiments, the antigen binding domain is targeted against a tumor antigen. In some embodiments, the antigen binding domain is a single chain variable fragment (scFv). In some embodiments, the intracellular signaling domain comprises a primary signaling domain and a costimulatory signaling domain. In some embodiments, the intracellular signaling domain comprises one or more signaling domains of CD3ζ, DAP10, DAP12, CD28, 4-1BB, or OX40. In some embodiments, the intracellular signaling domain comprises two or more signaling domains of CD3ζ, DAP10, DAP12, CD28, 4-1BB, or OX40. In some embodiments, the intracellular signaling domain comprises a primary signaling domain comprising a signaling domain of CD3ζ and a costimulatory signaling domain. In some embodiments, the costimulatory signaling domain is a signaling domain of CD28 or 4-1BB.

In some embodiments, the heterologous nucleic acid encoding the CAR is stably integrated into the genome of the cell. In some embodiments, the heterologous nucleic acid encoding the CAR is transiently expressed.

In some embodiments, the immunomodulator is an immunosuppressant. In some embodiments, the immunomodulator is an immunoactivator. In some embodiments, the immunomodulator is a cytokine. In some embodiments, the cytokine is secretable from the engineered NK cell. In some embodiments, the secretable cytokine is IL-2 or a biological portion thereof; IL-15 or a biological portion thereof; or IL-21 or a biological portion thereof; or combinations thereof. In other embodiments, the cytokine is membrane-bound. In some embodiments, the membrane-bound cytokine is membrane-bound IL-2 (mbIL-2); membrane-bound IL-15 (mbIL-15); membrane-bound IL-21 (mbIL-21); or combinations thereof

In some embodiments, the heterologous nucleic acid encoding the immunomodulator is stably integrated into the genome of the cell. In some embodiments, the heterologous nucleic acid encoding the immunomodulator is transiently expressed.

In some embodiments, the g-NK cell has a surface phenotype that is CD16^{pos}/CD57^{pos}/CD7^{dim/neg}/CD161^{neg}. In some embodiments, the g-NK cell further has a surface phenotype that is NKG2A^{neg}/CD161^{neg}. In some embodiments, the g-NK cell further is CD38^{neg}. In some embodiments, the g-NK cell has a surface phenotype that further is CD45^{pos}/CD3^{neg}/CD56^{pos}. In some embodiments, the g-NK cells comprise CD16 158V/V (V158). In some embodiments, the g-NK cells are CD16 158V/F.

In some embodiments, there is a composition comprising a plurality of engineered g-NK cells from any of the above described embodiments.

In some embodiments, greater than at or about 50% of the NK cells or total cells in the composition are g-NK cells. In some embodiments, greater than at or about 60% of the NK cells or total cells in the composition are g-NK cells. In some embodiments, greater than at or about 70% of the NK cells or total cells in the composition are g-NK cells. In some embodiments, greater than at or about 80% of the NK cells or total cells in the composition are g-NK cells. In some embodiments, greater than at or about 90% of the NK cells or total cells in the composition are g-NK cells. In some embodiments, greater than at or about 95% of the NK cells or total cells in the composition are g-NK cells.

In some embodiments, the plurality of engineered g-NK cells comprises greater than at or about 20% g-NK cells comprising a heterologous nucleic acid encoding the CAR. In some embodiments, the plurality of engineered g-NK cells comprises greater than at or about 30% g-NK cells comprising a heterologous nucleic acid encoding the CAR. In some embodiments, the plurality of engineered g-NK cells comprises greater than at or about 40% g-NK cells comprising a heterologous nucleic acid encoding the CAR. In some embodiments, the plurality of engineered g-NK cells comprises greater than at or about 50% g-NK cells comprising a heterologous nucleic acid encoding the CAR. In some embodiments, the plurality of engineered g-NK cells comprises greater than at or about g-NK 60% cells comprising a heterologous nucleic acid encoding the CAR. In some embodiments, the plurality of engineered g-NK cells comprises greater than at or about g-NK 70% cells comprising a heterologous nucleic acid encoding the CAR

In some embodiments, the total composition comprises greater than at or about 20% g-NK cells comprising a heterologous nucleic acid encoding the CAR. In some embodiments, the total composition comprises greater than at or about 30% g-NK cells comprising a heterologous nucleic acid encoding the CAR. In some embodiments, the total composition comprises greater than at or about 40% g-NK cells comprising a heterologous nucleic acid encoding the CAR. In some embodiments, the total composition comprises greater than at or about 50% g-NK cells comprising a heterologous nucleic acid encoding the CAR. In some embodiments, the total composition comprises greater than at or about 60% g-NK cells comprising a heterologous nucleic acid encoding the CAR. In some embodiments, the total composition comprises greater than at or about 70% g-NK cells comprising a heterologous nucleic acid encoding the CAR

In some embodiments, the plurality of engineered g-NK cells comprises greater than at or about 20% g-NK cells comprising a heterologous nucleic acid encoding the immunomodulator. In some embodiments, the plurality of engineered g-NK cells comprises greater than at or about 30% g-NK cells comprising a heterologous nucleic acid encoding the immunomodulator. In some embodiments, the plurality of engineered g-NK cells comprises greater than at or about 40% g-NK cells comprising a heterologous nucleic acid encoding the immunomodulator. In some embodiments, the plurality of engineered g-NK cells comprises greater than at or about 50% g-NK cells comprising a heterologous nucleic acid encoding the immunomodulator. In some embodiments, the plurality of engineered g-NK cells comprises greater than at or about 60% g-NK cells comprising a heterologous nucleic acid encoding the immunomodulator. In some embodiments, the plurality of engineered g-NK cells comprises greater than at or about 70% g-NK cells comprising a heterologous nucleic acid encoding the immunomodulator.

In some embodiments, the total composition comprises greater than at or about 20% g-NK cells comprising a heterologous nucleic acid encoding the immunomodulator. In some embodiments, the total composition comprises greater than at or about 30% g-NK cells comprising a heterologous nucleic acid encoding the immunomodulator. In some embodiments, the total composition comprises greater than at or about 40% g-NK cells comprising a heterologous nucleic acid encoding the immunomodulator. In some embodiments, the total composition comprises greater than at or about 50% g-NK cells comprising a heterologous nucleic acid encoding the immunomodulator. In some embodiments, the total composition comprises greater than at or about 60% g-NK cells comprising a heterologous nucleic acid encoding the immunomodulator. In some embodiments, the total composition comprises greater than at or about 70% g-NK cells comprising a heterologous nucleic acid encoding the immunomodulator.

In some embodiments, the plurality of engineered g-NK cells comprises greater than at or about 20% g-NK cells comprising a heterologous nucleic acid encoding the CAR and the heterologous nucleic acid encoding the immunomodulator. In some embodiments, the plurality of engineered g-NK cells comprises greater than at or about 30% g-NK cells comprising a heterologous nucleic acid encoding the CAR and the heterologous nucleic acid encoding the immunomodulator. In some embodiments, the plurality of engineered g-NK cells comprises greater than at or about 40% g-NK cells comprising a heterologous nucleic acid encoding the CAR and the heterologous nucleic acid encoding the immunomodulator. In some embodiments, the plurality of engineered g-NK cells comprises greater than at or about 50% g-NK cells comprising a heterologous nucleic acid encoding the CAR and the heterologous nucleic acid encoding the immunomodulator. In some embodiments, the plurality of engineered g-NK cells comprises greater than at or about 60% g-NK cells comprising a heterologous nucleic acid encoding the CAR and the heterologous nucleic acid encoding the CAR and the heterologous nucleic acid encoding the immunomodulator. In some embodiments, the plurality of engineered g-NK cells comprises greater than at or about 70% g-NK cells comprising a heterologous nucleic acid encoding the CAR and the heterologous nucleic acid encoding the immunomodulator.

In some embodiments, the total composition comprises greater than at or about 20% g-NK cells comprising a heterologous nucleic acid encoding the CAR and the heterologous nucleic acid encoding the immunomodulator. In some embodiments, the total composition comprises greater than at or about 30% g-NK cells comprising a heterologous nucleic acid encoding the CAR and the heterologous nucleic acid encoding the immunomodulator. In some embodiments, the total composition comprises greater than at or about 40% g-NK cells comprising a heterologous nucleic acid encoding the CAR and the heterologous nucleic acid encoding the immunomodulator. In some embodiments, the total composition comprises greater than at or about 50% g-NK cells comprising a heterologous nucleic acid encoding the CAR and the heterologous nucleic acid encoding the immunomodulator. In some embodiments, the total composition comprises greater than at or about 60% g-NK cells comprising a heterologous nucleic acid encoding the CAR and the heterologous nucleic acid encoding the immunomodulator. In some embodiments, the total composition comprises greater than at or about 70% g-NK cells comprising a heterologous nucleic acid encoding the CAR and the heterologous nucleic acid encoding the immunomodulator.

In some embodiments, greater than at or about 70% of the g-NK cells are positive for perforin and greater than at or about 70% of the g-NK cells are positive for granzyme B. In some embodiments, greater than at or about 80% of the g-NK cells are positive for perforin and greater than at or about 80% of the g-NK cells are positive for granzyme B. In some embodiments, greater than at or about 90% of the g-NK cells are positive for perforin and greater than at or about 90% of the g-NK cells are positive for granzyme B. In some embodiments, greater than at or about 95% of the g-NK cells are positive for perforin and greater than at or about 95% of the g-NK cells are positive for granzyme B.

In some embodiments, among the cells positive for perforin, the cells express a mean level of perforin as measured by intracellular flow cytometry that is, based on mean fluorescence intensity (MFI), at least at or about two times the mean level of perforin expressed by cells that are FcRγ^{pos}; and/or among the cells positive for granzyme B, the cells express a mean level of granzyme B as measured by intracellular flow cytometry that is, based on mean fluorescence intensity (MFI), at least at or about two times the mean level of granzyme B expressed by cells that are FcRγ^{pos}.

In some embodiments, greater than 10% of the cells in the composition are capable of degranulation against tumor target cells, optionally as measured by CD107a expression, optionally wherein the degranulation is measured in the absence of an antibody against the tumor target cells. In some embodiments, greater than 10% of the cells in the composition are capable of producing interferon-gamma or TNF-alpha against tumor target cells, optionally wherein the interferon-gamma or TNF-alpha is measured in the absence of an antibody against the tumor target cells.

In some embodiments, among the cells in the composition, greater than at or about 15% produce an effector cytokine in the presence of cells expressing a target antigen (target cells) and an antibody directed against the target antigen (anti-target antibody). In some embodiments, among the cells in the composition, greater than at or about 20% produce an effector cytokine in the presence of cells expressing a target antigen (target cells) and an antibody directed against the target antigen (anti-target antibody). In some embodiments, among the cells in the composition, greater than at or about 30% produce an effector cytokine in the presence of cells expressing a target antigen (target cells) and an antibody directed against the target antigen (anti-target antibody). In some embodiments, among the cells in the composition, greater than at or about 40% produce an effector cytokine in the presence of cells expressing a target antigen (target cells) and an antibody directed against the target antigen (anti-target antibody). In some embodiments, among the cells in the composition, greater than at or about 50% produce an effector cytokine in the presence of cells expressing a target antigen (target cells) and an antibody directed against the target antigen (anti-target antibody).

In some embodiments, among the total cells in the composition or among the g-NK cells in the composition, greater than at or about 30% are positive for NKG2C (NKG2C^{pos}) and/or greater than at or about 50% are negative or low for NKG2A (NKG2A^{neg}). In some embodiments, among the total cells in the composition or among the g-NK cells in the composition, greater than at or about 35% are positive for NKG2C (NKG2C^{pos}) and/or greater than at or about 60% are negative or low for NKG2A (NKG2A^{neg}). among the total cells in the composition or among the g-NK cells in the composition, greater than at or about 40% are positive for NKG2C (NKG2C^{pos}) and/or greater than at or about 70% are negative or low for NKG2A (NKG2A^{neg}). In some embodiments, among the total cells in the composition or among the g-NK cells in the composition, greater than at or about 45% are positive for NKG2C (NKG2C^{pos}) and/or greater than at or about 80% are negative or low for NKG2A (NKG2A^{neg}). In some embodiments, among the total cells in the composition or among the g-NK cells in the composition, greater than at or about 50% are positive for NKG2C (NKG2C^{pos}) and/or greater than at or about 85% are negative or low for NKG2A (NKG2A^{neg}). In some embodiments, among the total cells in the composition or among the g-NK cells in the composition, greater than at or about 55% are positive for NKG2C (NKG2C^{pos}) and/or greater than at or about 90% are negative or low for NKG2A (NKG2A^{neg}). In some embodiments, among the total cells in the composition, or among the g-NK cells in the composition, greater than at or about 60% are positive for NKG2C (NKG2C^{pos}) and/or greater than at or about 95% are negative or low for NKG2A (NKG2A^{neg}).

In some embodiments, among the total cells in the composition or among the g-NK cells in the composition, greater than at or about 50% are CD38^{neg}. In some embodiments, among the total cells in the composition or among the g-NK cells in the composition, greater than at or about 60% are CD38^{neg}. In some embodiments, among the total cells in the composition or among the g-NK cells in the composition, greater than at or about 70% are CD38^{neg}. In some embodiments, among the total cells in the composition or among the g-NK cells in the composition, greater than at or about 80% are CD38^{neg}. In some embodiments, among the total cells in the composition or among the g-NK cells in the composition, greater than at or about 90% are CD38^{neg}.

In some embodiments, among the total cells in the composition or among the g-NK cells in the composition, greater than at or about 50% are CD16^{pos}/CD57^{pos}/CD7^{dim/neg}/CD161^{neg}. In some embodiments, among the total cells in the composition or among the g-NK cells in the composition, greater than at or about 60% are CD16^{pos}/CD57^{pos}/CD7^{dim/neg}/CD161^{neg}. In some embodiments, among the total cells in the composition or among the g-NK cells in the composition, greater than at or about 70% are CD16^{pos}/CD57^{pos}/CD7^{dim/neg}/CD161^{neg}. In some embodiments, among the total cells in the composition or among the g-NK cells in the composition, greater than at or about 80% are CD16^{pos}/CD57^{pos}/CD7^{dim/neg}/CD161^{neg}. In some embodiments, among the total cells in the composition or among the g-NK cells in the composition, greater than at or about 90% are CD16^{pos}/CD57^{pos}/CD7^{dim/neg}/CD161^{neg}.

In some embodiments, among the total cells in the composition or among the g-NK cells in the composition, greater than at or about 50% are NKG2A^{neg}/CD161^{neg}. In some embodiments, among the total cells in the composition or among the g-NK cells in the composition, greater than at or about 60% are NKG2A^{neg}/CD161^{neg}. In some embodiments, among the total cells in the composition or among the g-NK cells in the composition, greater than at or about 70% are NKG2A^{neg}/CD161^{neg}. In some embodiments, among the total cells in the composition or among the g-NK cells in the composition, greater than at or about 80% are NKG2A^{neg}/CD161^{neg}. In some embodiments, among the total cells in the composition or among the g-NK cells in the composition, greater than at or about 90% are NKG2A^{neg}/CD161^{neg}.

In some embodiments, the plurality of g-NK cells are CD16 158 (V/V) (V158). In some embodiments, the plurality of g-NK cells are CD16 158V/F.

In some embodiments, the composition comprises at least or about at least 10⁸ cells. In some embodiments, the number of g-NK cells in the composition is from at or about 10⁸ to at or about 10¹² cells. In some embodiments, the number of g-NK cells in the composition is from at or about 10⁸ to at or about 10¹¹ cells. In some embodiments, the number of g-NK cells in the composition is from at or about 10⁸ to at or about 10¹⁰ cells. In some embodiments the number of g-NK cells in the composition is from at or about 10⁸ to at or about 10⁹ cells. In some embodiments, the number of g-NK cells in the composition is from at or about 10⁹ cells to at or about 10¹² cells. In some embodiments, the number of g-NK cells in the composition is from at or about 10⁹ to at or about 10¹¹ cells. In some embodiments, the number of g-NK cells in the composition is from at or about 10⁹ to at or about 10¹⁰ cells. In some embodiments, the number of g-NK cells in the composition is from at or about 10¹⁰ to at or about 10¹² cells. In some embodiments, the number of g-NK cells in the composition is from at or about 10¹⁰ to at or about 10¹¹ cells. In some embodiments, the number of g-NK cells in the composition is from at or about 10¹¹ to at or about 10¹² cells.

In some embodiments, the number of g-NK cells in the composition is or is about 5×10⁸ cells. In some embodiments, the number of g-NK cells in the composition is or is about 1×10⁹ cells. In some embodiments, the number of g-NK cells in the composition is or is about 5×10¹⁰ cells. In some embodiments, the number of g-NK cells in the composition is or is about 1×10¹⁰ cells

In some embodiments, the volume of the composition is between at or about 50 mL and at or about 500 mL. In some embodiments, the volume of the composition is optionally at or about 200 mL.

In some embodiments, the cells in the composition are from a single donor subject that have been expanded from the same biological sample.

In some embodiments, the composition is a pharmaceutical composition. In some embodiments, the composition comprises a pharmaceutically acceptable excipient. In some embodiments, the composition is formulated in a serum-free cryopreservation medium comprising a cryoprotectant. In some embodiments, the cryoprotectant is DMSO and the cryopreservation medium is 5% to 10% DMSO (v/v). In some embodiments, the cryoprotectant is or is about 10% DMSO (v/v).

In some embodiments, the composition is sterile. In some embodiments, the composition comprises a sterile bag. In some embodiments, the bag is a cryopreservation-compatible bag.

Also provided herein is a method of producing a genetically engineered g-NK cell, comprising introducing into an NK cell deficient in expression of FcRγ chain (g-NK cells) a heterologous nucleic acid encoding a chimeric antigen receptor (CAR).

In some aspects, provided herein is a method of producing a genetically engineered g-NK cell comprising introducing into the g-NK cell a heterologous nucleic acid encoding an immunomodulator, thereby producing a genetically engineered g-NK cell.

In some aspects, provided herein is a method of producing a genetically engineered g-NK cell comprising: (a) introducing into an NK cell deficient in expression of FcRγ chain (g-NK cells) a heterologous nucleic acid encoding a chimeric antigen receptor (CAR), and (b) introducing into the g-NK cell a heterologous nucleic acid encoding an immunomodulator, thereby producing a genetically engineered g-NK cell, wherein steps (a) and (b) are carried out simultaneously or sequentially in any order.

In some embodiments, the CAR comprises 1) an antigen binding domain; 2) a flexible linker (spacer); 3) a transmembrane region; and 4) an intracellular signaling domain. In some embodiments, the antigen binding domain is targeted against a tumor antigen. In some embodiments, the antigen binding domain is a single chain variable fragment (scFv). In some embodiments, the intracellular signaling domain comprises one or more signaling domain from CD3ζ, DAP10, DAP12, CD28, 4-1BB, or OX40. In some embodiments, intracellular signaling domain comprises two or more signaling domains from CD3ζ, DAP10, DAP12, CD28, 4-1BB, or OX40.

In some embodiments, the intracellular signaling domain comprises a primary signaling domain comprising a signaling domain of CD3ζ and a costimulatory signaling domain. In some embodiments, the costimulatory signaling domain is a signaling domain of CD28 or 4-1BB.

In some embodiments, the heterologous nucleic acid encoding the CAR is introduced under conditions for stable integration into the genome of the g-NK cell. In some embodiments, the heterologous nucleic acid encoding the CAR is comprised in a viral vector and is introduced into the g-NK cell by transduction. In some embodiments, the viral vector is a lentiviral vector.

In some embodiments, the nucleic acid encoding the CAR is introduced under conditions for transient expression in the g-NK cell. In some embodiments, the nucleic acid encoding the CAR is introduced to the g-NK cell via a lipid nanoparticle. In some embodiments, the nucleic acid encoding the CAR is DNA. In some embodiments, the nucleic acid encoding the CAR is RNA. In some embodiments, the RNA is mRNA. In some embodiments, the RNA is self-amplifying mRNA. In some embodiments, the nucleic acid is introduced to the g-NK cell via electroporation.

In some embodiments, the immunomodulator is an immunosuppressant. In some embodiments, the immunomodulator is an immunoactivator. In some embodiments, the immunomodulator is a cytokine. In some embodiments, the cytokine is secretable from the engineered NK cell. In some embodiments, the secretable cytokine is IL-2 or a biologically active portion thereof; IL-15 or a biologically active portion thereof; IL-21 or a biologically active portion thereof or combinations thereof. In other embodiments, the cytokine is membrane bound. In some embodiments, the membrane-bound cytokine is membrane bound IL-2 (mbIL-2); membrane-bound IL-15 (mbIL-15); or membrane bound IL-21 (mbIL-21) or combinations thereof.

In some embodiments, the nucleic acid encoding the immunomodulator is introduced under conditions for stable integration into the genome of the g-NK cell. In some embodiments, the nucleic acid encoding an immunomodulator is comprised in a viral vector and is introduced into the g-NK cell by transduction. In some embodiments, the viral vector is a lentiviral vector. In some embodiments, the nucleic acid encoding the immunomodulator is introduced under conditions for transient expression in the g-NK cell. In some embodiments, the nucleic acid encoding the immunomodulator is introduced by non-viral delivery. In some embodiments, the nucleic acid encoding the immunomodulator is introduced to the g-NK cell via a lipid nanoparticle. In some embodiments, the nucleic acid encoding the immunomodulator is DNA. In some embodiments, the nucleic acid encoding the immunomodulator is RNA. In some embodiments, the RNA is mRNA. In some embodiments, the RNA is self-amplifying RNA. In some embodiments, the nucleic acid encoding the immunomodulator is introduced to the g-NK cell via electroporation.

In some embodiments, the nucleic acid encoding the CAR and the nucleic acid encoding the immunomodulator are encoded from the same polynucleotide and introduced together. In some embodiments, the nucleic acid encoding the CAR and the nucleic acid encoding the immunomodulator are separated by a multicistronic element of the polynucleotide sequence. In some embodiments, the multicistronic element is a self-cleaving peptide selected from the group consisting of T2A, P2A and F2A. In some embodiments, the nucleic acid encoding the CAR and the nucleic acid encoding the immunomodulator are introduced simultaneously during an ex vivo process for expanding a population enriched in g-NK cells.

In some embodiments, the g-NK cell composition has been produced by ex vivo expansion of NK cells enriched from a biological sample from a subject that are either (i) negative or low for CD3 and positive for CD57 (CD3^{neg}CD57^{pos}) or (ii) negative or low for CD3 and positive for CD56 (CD3^{neg}CD56^{pos}), wherein the enriched NK cells are cultured with irradiated HLA-E+ feeder cells and one or more recombinant cytokines. In some embodiments, the one or more recombinant cytokine is selected from an effective amount of SCF, GSK3i, FLT3, IL-2, IL-6, IL-7, IL-15, IL-12, IL-18, IL-21, IL-27, or combinations thereof. In some embodiments, the culturing is performed in the presence of two or more recombinant cytokines, wherein at least one recombinant cytokine is interleukin (IL)-2 and at least one recombinant cytokine is IL-21. In some embodiments, the one or more recombinant cytokine comprises IL-21.

In some embodiments, the introducing is carried out during a method for expanding FcRγ-deficient NK (g-NK) cells, said method comprising: (A) obtaining a population of primary human cells enriched for natural killer (NK) cells, wherein the population enriched for NK cells is selected from a biological sample from a human subject; and (B) culturing the population of engineered NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21; wherein the introducing the nucleic acid encoding the CAR is performed after step (A) and prior to, during or after step (B), thereby producing a population of engineered NK cells; and wherein the method produces an expanded population of engineered NK cells that are enriched in g-NK cells and comprise cells engineered with a CAR

In some embodiments, the introducing is carried out during a method for expanding FcRγ-deficient NK cells (g-NK), said method comprising: (A) obtaining a population of primary human cells enriched for natural killer (NK) cells, wherein the population enriched for NK cells is selected from a biological sample from a human subject; (B) introducing into the population of enriched NK cells the nucleic acid encoding a CAR, thereby producing a population of engineered NK cells; and (C) culturing the population of engineered NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21, thereby producing an expanded population of NK cells; and wherein the method produces an expanded population of engineered NK cells that are enriched in g-NK cells and comprise cells engineered with a CAR

In some embodiments, the introducing is carried out during a method for expanding FcRγ-deficient NK cells (g-NK), said method comprising: (A) obtaining a population of primary human cells enriched for natural killer (NK) cells, wherein the population enriched for NK cells is selected from a biological sample from a human subject; and (B) culturing the population of enriched NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21, thereby producing an expanded population of NK cells; and (C) introducing into NK cells of the expanded population of NK cells the nucleic acid encoding a CAR, wherein the method produces an expanded population of engineered NK cells that are enriched in g-NK cells and comprises cells engineered with a CAR

In some embodiments, the introducing is carried out during a method for expanding FcRγ-deficient NK cells (g-NK), said method comprising: (A) obtaining a population of primary human cells enriched for natural killer (NK) cells, wherein the population enriched for NK cells is selected from a biological sample from a human subject; and (B) performing a first expansion comprising culturing the population of enriched NK cells in culture medium under conditions for expanding the NK cells to produce a first expanded population of NK cells; (C) introducing into NK cells of the first expanded population of NK cells the nucleic acid encoding a CAR, thereby producing a population of engineered NK cells; and (D) performing a second expansion comprising culturing the population of engineered NK cells under conditions for further expansion of the NK cells, wherein the first expansion and/or second expansion comprise culturing the population of enriched NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21; and wherein the method produces an expanded population of engineered NK cells that are enriched in g-NK cells and comprise cells engineered with a CAR.

In some embodiments, the g-NK cell composition has been produced by ex vivo expansion of NK cells enriched from a biological sample from a subject that are either (i) negative or low for CD3 and positive for CD57 (CD3^{neg}CD57^{pos}) or (ii) negative or low for CD3 and positive for CD56 (CD3^{neg}CD56^{pos}), wherein the enriched NK cells cultured with irradiated HLA-E+ feeder cells and one or more recombinant cytokines. In some embodiments, the one or more recombinant cytokine is selected from an effective amount of SCF, GSK3i, FLT3, IL-2, IL-6, IL-7, IL-15, IL-12, IL-18, IL-21, IL-27, or combinations thereof. In some embodiments, the culturing is performed in the presence of two or more recombinant cytokines, wherein at least one recombinant cytokine is interleukin (IL)-2 and at least one recombinant cytokine is IL-21.

In some embodiments, the introducing is carried out during a method for expanding FcRγ-deficient NK (g-NK) cells, said method comprising: (A) obtaining a population of primary human cells enriched for natural killer (NK) cells, wherein the population enriched for NK cells is selected from a biological sample from a human subject; and (B) culturing the population of engineered NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21; wherein the introducing the nucleic acid encoding the immunomodulator is performed after step (A) and prior to, during or after step (B), thereby producing a population of engineered NK cells; and wherein the method produces an expanded population of engineered NK cells that are enriched in g-NK cells and comprises cells engineered with an immunomodulator.

In some embodiments, the introducing is carried out during a method for expanding FcRγ-deficient NK cells (g-NK), said method comprising: (A) obtaining a population of primary human cells enriched for natural killer (NK) cells, wherein the population enriched for NK cells is selected from a biological sample from a human subject; (B) introducing into the population of enriched NK cells the nucleic acid encoding an immunomodulatory, thereby producing a population of engineered NK cells; and (C) culturing the population of engineered NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21, thereby producing an expanded population of NK cells; and wherein the method produces an expanded population of engineered NK cells that are enriched in g-NK cells and comprise cells engineered with an immunomodulator.

In some embodiments, the introducing is carried out during a method for expanding FcRγ-deficient NK cells (g-NK), said method comprising: (A) obtaining a population of primary human cells enriched for natural killer (NK) cells, wherein the population enriched for NK cells is selected from a biological sample from a human subject; and (B) culturing the population of enriched NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21, thereby producing an expanded population of NK cells; and (C) introducing into NK cells of the expanded population of NK cells the nucleic acid encoding an immunomodulator, wherein the method produces an expanded population of engineered NK cells that are enriched in g-NK cells and comprise cells engineered with an immunomodulator.

In some embodiments, the introducing is carried out during a method for expanding FcRγ-deficient NK (g-NK) cells, said method comprising: (A) obtaining a population of primary human cells enriched for natural killer (NK) cells, wherein the population enriched for NK cells is selected from a biological sample from a human subject; and (B) culturing the population of engineered NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21; wherein the introducing (i) the nucleic acid encoding the CAR and/or (ii) the nucleic acid encoding the immunomodulatory is performed after step (A) and prior to, during or after step (B), wherein steps (i) and (ii) are carried out simultaneously or sequentially in any order, thereby producing a population of engineered NK cells; and wherein the method produces an expanded population of engineered NK cells that are enriched in g-NK cells and comprise cells engineered with a CAR and an immunomodulator.

In some embodiments, the introducing is carried out during a method for expanding FcRγ-deficient NK cells (g-NK), said method comprising: (A) obtaining a population of primary human cells enriched for natural killer (NK) cells, wherein the population enriched for NK cells is selected from a biological sample from a human subject; (B) introducing into the population of enriched NK cells (i) the nucleic acid encoding a CAR, and (ii) the nucleic acid encoding an immunomodulator, wherein steps (i) and (ii) are carried out simultaneously or sequentially in any order, thereby producing a population of engineered NK cells; and (C) culturing the population of engineered NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21, thereby producing an expanded population of NK cells; and wherein the method produces an expanded population of engineered NK cells that are enriched in g-NK cells and comprise cells engineered with a CAR and an immunomodulator.

In some embodiments, the introducing is carried out during a method for expanding FcRγ-deficient NK cells (g-NK), said method comprising: (A) obtaining a population of primary human cells enriched for natural killer (NK) cells, wherein the population enriched for NK cells is selected from a biological sample from a human subject; and (B) culturing the population of enriched NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21, thereby producing an expanded population of NK cells; and (C) introducing into NK cells of the expanded population of NK cells (i) the nucleic acid encoding a CAR, and (ii) the nucleic acid encoding an immunomodulator, wherein steps (i) and (ii) are carried out simultaneously or sequentially in any order, wherein the method produces an expanded population of engineered NK cells that are enriched in g-NK cells and comprise cells engineered with a CAR and an immunomodulator.

In some embodiments, the introducing is carried out during a method for expanding FcRγ-deficient NK cells (g-NK), said method comprising: (A) obtaining a population of primary human cells enriched for natural killer (NK) cells, wherein the population enriched for NK cells is selected from a biological sample from a human subject; and (B) performing a first expansion comprising culturing the population of enriched NK cells in culture medium under conditions for expanding the NK cells to produce a first expanded population of NK cells; (C) introducing into NK cells of the first expanded population of NK cells (i) the nucleic acid encoding a CAR, and (ii) the nucleic acid encoding an immunomodulator, wherein steps (i) and (ii) are carried out simultaneously or sequentially in any order, thereby producing a population of engineered NK cells; and (D) performing a second expansion comprising culturing the population of engineered NK cells under conditions for further expansion of the NK cells, wherein the first expansion and/or second expansion comprise culturing the population of enriched NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21; and wherein the method produces an expanded population of engineered NK cells that are enriched in g-NK cells and comprise cells engineered with a CAR and an immunomodulator.

In some embodiments, the population of primary human cells enriched for NK cells are obtained by selecting from a biological sample from a human subject cells that are either (i) negative or low for CD3 and positive for CD57 (CD3^{neg}CD57^{pos}) or (ii) negative or low for CD3 and positive for CD56 (CD3^{neg}CD56^{pos}).

In some embodiments, the population enriched for NK cells are cells further selected for cells positive for NKG2C (NKG2C^{pos}); the population enriched for NK cells are cells further selected for cells negative or low for NKG2A (NKG2A^{neg}); or the population enriched for NK cells are cells further selected for cells positive for NKG2C and negative or low for NKG2A (NKG2C^{pos}NKG2A^{neg}).

In some embodiments, the human is a subject in which at least at or about 20% of natural killer (NK) cells in a peripheral blood sample from the subject are positive for NKG2C (NKG2C^{pos}) and at least 70% of NK cells in the peripheral blood sample are negative or low for NKG2A (NKG2A^{neg}). In some embodiments, the subject is CMV-seropositive.

In some embodiments, the percentage of g-NK cells among NK cells in the biological sample from the subject is greater than at or about 5%. In some embodiments, the percentage of g-NK cells among NK cells in the biological sample from the subject is greater than at or about 10%. In some embodiments, the percentage of g-NK cells among NK cells in the biological sample from the subject is greater than at or about 30%.

In some embodiments, the percentage of g-NK cells among the population of enriched NK cells is between at or about 20% and at or about 90%. In some embodiments, the percentage of g-NK cells among the population of enriched NK cells is between at or about 40% and at or about 90%. In some embodiments, the percentage of g-NK cells among the population of enriched NK cells is between at or about 60% and at or about 90%.

In some embodiments, the population enriched for NK cells are cells selected from the biological sample that are negative or low for CD3 and positive for CD57 (CD3^{neg}CD57^{pos}). In some embodiments, the population enriched for NK cells are cells selected from the biological sample that are negative or low for CD3 and positive for CD56 (CD3^{neg}CD56^{pos}).

In some embodiments, the two or more recombinant cytokines further comprises an effective amount of SCF, GSK3i, FLT3, IL-6, IL-7, IL-15, IL-12, IL-18, IL-27, or combinations thereof. In some embodiments, the recombinant cytokines are IL-21 and IL-2. In some embodiments, the recombinant cytokines are IL-21, IL-2, and IL-15.

In some embodiments, recombinant IL-21 is added to the culture medium during at least a portion of the culturing at a concentration that is from at or about 10 ng/mL to at or about 100 ng/mL. In some embodiments, recombinant IL-21 is added to the culture medium during at least a portion of the culturing at a concentration that is at or about 25 ng/mL. In some embodiments, recombinant IL-2 is added to the culture medium during at least a portion of the culturing at a concentration that is from at or about 10 IU/mL to at or about 500 IU/mL. In some embodiments, recombinant IL-2 is added to the culture medium during at least a portion of the culturing at a concentration that is at or about 100 IU/mL. In some embodiments, recombinant IL-2 is added to the culture medium during at least a portion of the culturing at a concentration that is at or about 500 IU/mL. In some embodiments, recombinant IL-15 is added to the culture medium during at least a portion of the culturing at a concentration that is from at or about 1 ng/mL to 50 ng/mL. In some embodiments, recombinant IL-15 is added to the culture medium during at least a portion of the culturing at a concentration that is at or about 10 ng/mL.

In some embodiments, the recombinant cytokines are added to the culture medium beginning at or about the initiation of the culturing. In some embodiments, the method further comprises exchanging the culture medium one or more times during the culturing, wherein at each exchange of the culture medium, fresh media containing the recombinant cytokines is added. In some embodiments, the exchanging of the culture medium is carried out every two or three days for the duration of the culturing. In some embodiments, exchanging the media is performed after a first expansion without media exchange for up to 5 days, optionally after a first expansion without media exchange for up to 5 days.

In some embodiments, the human subject has the CD16 158V/V NK cell genotype or the CD16 158V/F NK cell genotype, optionally wherein the biological sample is from a human subject selected for the CD16 158V/V NK cell genotype or the CD16 158V/F NK cell genotype.

In some embodiments, the biological sample is or comprises peripheral blood mononuclear cells (PBMCs), optionally is a blood sample, an apheresis sample or a leukapheresis sample. In some embodiments, the HLA-E+ feeder cells are K562 cells transformed with HLA-E (K562-HLA-E). In some embodiments, the HLA-E+ feeder cells are 221.AEH cells.

In some embodiments, the ratio of the irradiated HLA-E+ feeder cells to enriched NK cells is between 1:1 and 5:1, inclusive. In some embodiments, the ratio of the irradiated HLA-E+ feeder cells to enriched NK cells is between 1:1 and 3:1, inclusive. In some embodiments, the ratio of the irradiated HLA-E+ feeder cells to enriched NK cells optionally is or is about 2.5:1. In some embodiments, the ratio of irradiated HLA-E+ feeder cells to enriched NK cells optionally is or is about 2:1. In some embodiments, the ratio of irradiated HLA-E+ feeder cells to enriched NK cells optionally is about 1:1.

In some embodiments, the recombinant cytokines added to the culture medium during at least a portion of the culturing are 500 IU/mL IL-2, 10 ng/mL IL-15, and 25 ng/mL IL-21.

In some embodiments, the population of enriched NK cells comprises between at or about 2.0×10⁵ enriched NK cells and at or about 5.0×10⁷ enriched NK cells, inclusive. In some embodiments, the population of enriched NK cells comprises between at or about 1.0×10⁶ enriched NK cells and at or about 1.0×10⁸ enriched NK cells, inclusive. In some embodiments, the population of enriched NK cells comprises between at or about 1.0×10⁷ enriched NK cells and at or about 5.0×10⁸ enriched NK cells, inclusive. In some embodiments, the population of enriched NK cells comprises between at or about 1.0×10⁷ enriched NK cells and at or about 1.0×10⁹ enriched NK cells, inclusive. In some embodiments, the population of enriched NK cells optionally comprises at or about 1.0×10⁶ enriched NK cells.

In some embodiments, the population of enriched NK cells at the initiation of the culturing is at a concentration of between or between about 0.05×10⁶ enriched NK cells/mL and 1.0×10⁶ enriched NK cells/mL. In some embodiments, the population of enriched NK cells at the initiation of the culturing is at a concentration of between or between about 0.05×10⁶ enriched NK cells/mL and 0.5×10⁶ enriched NK cells/mL. In some embodiments, the population of enriched NK cells at the initiation of culturing is optionally at a concentration of or about 0.2×10⁶ enriched NK cells/mL.

In some embodiments, the culturing is carried out until a time at which the method achieves expansion of at least or at least about 2.50×10⁸ g-NK cells. In some embodiments, the culturing is carried out until a time at which the method achieves expansion of at least or at least about 5.00×10⁸ g-NK cells. In some embodiments, the culturing is carried out until a time at which the method achieves expansion of at least or at least about 1.0×10⁹ g-NK cells. In some embodiments, the culturing is carried out until a time at which the method achieves expansion of at least or at least about 5.0×10⁹ g-NK cells.

In some embodiments, the culturing is carried out for or about or at least or at least about 5 days. In some embodiments, the culturing is carried out for or about or at least or at least about 6 days. In some embodiments, the culturing is carried out for or about or at least or at least about 7 days. In some embodiments, the culturing is carried out for or about or at least or at least about 8 days. In some embodiments, the culturing is carried out for or about or at least or at least about 9 days. In some embodiments, the culturing is carried out for or about or at least or at least about 10 days. In some embodiments, the culturing is carried out for or about or at least or at least about 11 days. In some embodiments, the culturing is carried out for or about or at least or at least about 12 days. In some embodiments, the culturing is carried out for or about or at least or at least about 13 days. In some embodiments, the culturing is carried out for or about or at least or at least about 14 days. In some embodiments, the culturing is carried out for or about or at least or at least about 15 days. In some embodiments, the culturing is carried out for or about or at least or at least about 16 days. In some embodiments, the culturing is carried out for or about or at least or at least about 17 days. In some embodiments, the culturing is carried out for or about or at least or at least about 18 days. In some embodiments, the culturing is carried out for or about or at least or at least about 19 days. In some embodiments, the culturing is carried out for or about or at least or at least about 20 days. In some embodiments, the culturing is carried out for or about or at least or at least about 21 days. In some embodiments, the culturing is carried out for or about or at least or at least about 22 days. In some embodiments, the culturing is carried out for or about or at least or at least about 23 days. In some embodiments, the culturing is carried out for or about or at least or at least about 24 days. In some embodiments, the culturing is carried out for or about or at least or at least about 25 days.

In some embodiments, the method further comprises collecting the expanded population of engineered NK cells produced by the method. In some embodiments, the method further comprises formulating the expanded population of engineered NK cells in a pharmaceutically acceptable excipient. In some embodiments, the method further comprises formulating the expanded population of engineered NK cells with a serum-free cryopreservation medium comprising a cryoprotectant. In some embodiments, the cryoprotectant is DMSO. In some embodiments, the cryoprotectant optionally is DMSO and the cryopreservation medium is 5% to 10% DMSO (v/v), optionally is or is about 10% DMSO (v/v).

In some embodiments, among the expanded population of engineered NK cells produced by the method, greater than 50% of the population are FcRγ^{neg}. In some embodiments, among the expanded population of engineered NK cells produced by the method, greater than 60% of the population are FcRγ^{neg}. In some embodiments, among the expanded population of engineered NK cells produced by the method, greater than 70% of the population are FcRγ^{neg}. In some embodiments, among the expanded population of engineered NK cells produced by the method, greater than 80% of the population are FcRγ^{neg}. In some embodiments, among the expanded population of engineered NK cells produced by the method, greater than 90% of the population are FcRγ^{neg}. In some embodiments, among the expanded population of engineered NK cells produced by the method, greater than 95% of the population are FcRγ^{neg}.

In some embodiments, a plurality of engineered NK cells are produced by the method of any of the above embodiments.

Also provided herein are methods of treating a disease or condition in a subject. In some embodiments, an effective amount of cells of the composition of any one of the above embodiments is administered to an individual in need thereof.

In some embodiments, the disease or condition is an inflammatory condition. In some embodiments, the disease or condition is an infection. In some embodiments, the disease or condition is cancer. In some embodiments, the disease or condition is a cancer. In some embodiments, the cancer is leukemia. In some embodiments, the cancer is a lymphoma. In some embodiments, the cancer is a myeloma. In some embodiments, the disease or condition is a cancer and the cancer comprises a solid tumor.

In some embodiments, the cancer is adenocarcinoma of the stomach or gastroesophageal junction. In some embodiments, the cancer is a bladder cancer. In some embodiments, the cancer is a breast cancer. In some embodiments, the cancer is a brain cancer. In some embodiments, the cancer is a cervical cancer. In some embodiments, the cancer is a colorectal cancer. In some embodiments, the cancer is an endocrine/neuroendocrine cancer. In some embodiments, the cancer is a head and neck cancer. In some embodiments, the cancer is a gastrointestinal stromal cancer. In some embodiments, the cancer is a giant cell tumor of the bone. In some embodiments, the cancer is a kidney cancer. In some embodiments, the cancer is a liver cancer. In some embodiments, the cancer is a lung cancer. In some embodiments, the cancer is a neuroblastoma. In some embodiments, the cancer is an ovarian epithelial/fallopian tube/primary peritoneal cancer. In some embodiments, the cancer is a pancreatic cancer. In some embodiments, the cancer is a prostate cancer. In some embodiments, the cancer is a skin cancer. In some embodiments, the cancer is a soft tissue carcinoma.

In some embodiments, the composition is administered as a monotherapy. In some embodiments, the method further comprises administering an additional agent to the individual for treating the disease or condition. In some embodiments, the additional agent is an antibody or an Fc-fusion protein. In some embodiments, the additional agent is an antibody that is a monoclonal antibody. In some embodiments, the antibody is a full-length antibody. In some embodiments, the antibody is an IgG1 antibody. In some embodiments, the disease or condition is a cancer and the additional agent, optionally the antibody, recognizes a tumor antigen associated with the cancer. In some embodiments, the antibody recognizes a tumor antigen associated with the cancer.

In some embodiments, the method comprises administering from at or about 1×10⁵ to at or about 50×10⁹ cells of the g-NK cell composition to the individual. In some embodiments, the method comprises administering from at or about 1×10⁸ cells to at or about 50×10⁹ NK cells of the g-NK cell composition to the individual. In some embodiments, the method optionally comprises administering about at or about 5×10⁸ cells of the g-NK cell composition, at or about 5×10⁹ cells of the g-NK cell composition or at or about 10×10⁹ cells of the g-NK cell composition to the individual.

In some embodiments, the method further comprises administering exogenous cytokine support to facilitate expansion or persistence of the administered NK cells in vivo in the subject. In some embodiments, the exogenous cytokine optionally is or optionally comprises IL-15.

In some embodiments, prior to the administration of the dose of g-NK cells, the subject has received a lymphodepleting therapy. In some embodiments, the therapy comprises fludarabine and/or cyclophosphamide. In some embodiments, the lymphodepleting comprises the administration of fludarabine at or about 20-40 mg/m² body surface area of the subject. In some embodiments, the lymphodepleting optionally comprises at or about 30 mg/m², daily, for 2-4 days, and/or cyclophosphamide at or about 200-400 mg/m² body surface area of the subject, optionally at or about 300 mg/m², daily, for 2-4 days. In some embodiments, the lymphodepleting therapy comprises fludarabine and cyclophosphamide. In some embodiments, the lymphodepleting therapy comprises the administration of fludarabine at or about 30 mg/m² body surface area of the subject, daily, and cyclophosphamide at or about 300 mg/m² body surface area of the subject, daily, each for 2-4 days, optionally 3 days.

In some embodiments, administration of a dose of g-NK cells is initiated within two weeks after initiation of the lymphodepleting therapy. In some embodiments, administration of a dose of g-NK cells is initiated at or about two weeks after initiation of the lymphodepleting therapy.

In some embodiments, the individual is a human. In some embodiments, the NK cells in the composition are allogenic to the individual. In some embodiments, the NK cells in the composition are autologous to the subject.

### BRIEF DESCRIPTION OF THE DRAWINGS

FIG. 1A and FIG. 1B depict the expansion of g-NK cells expanded in the presence of 221.AEH or K562-mbIL15-41BBL feeder cells with or without IL-21 included in the NK cell media. FIG. 1A shows total NK cell counts. FIG. 1B shows g-NK cell counts after 21 days of expansion.

FIG. 2A and FIG. 2B depict daratumumab- and elotuzumab-mediated cytotoxic activity 21 days post-expansion of g-NK cells expanded in the presence of 221.AEH or K562-mbIL15-41BBL feeder cells with or without IL-21 included in the NK cell media. FIG. 2A shows g-NK cell cytotoxicity against the LP1 cell line. FIG. 2B shows g-NK cell cytotoxicity against the MM.1S cell line.

FIG. 3A-3D depict daratumumab- and elotuzumab-mediated degranulation levels (CD107a^{pos}) of g-NK cells expanded in the presence of 221.AEH or K562-mbIL15-41BBL feeder cells with or without IL-21 included in the NK cell media. FIG. 3A shows g-NK cell degranulation levels 13 days post-expansion against the LP1 cell line. FIG. 3B shows g-NK cell degranulation levels 13 days post-expansion against the MM.1S cell line. FIG. 3C shows g-NK cell degranulation levels 21 days post-expansion against the LP1 cell line. FIG. 3D shows g-NK cell degranulation levels 21 days post-expansion against the MM.1S cell line.

FIG. 4A-4D depict levels of perforin and granzyme B expression in g-NK cells expanded in the presence of 221.AEH or K562-mbIL 15-41BBL feeder cells with or without IL-21 included in the NK cell media. FIG. 4A shows perforin and granzyme B expression 13 days post-expansion as percentages of g-NK cells. FIG. 4B shows total perforin and granzyme B expression 13 days post-expansion. FIG. 4C shows perforin and granzyme B expression 21 days post-expansion as percentages of g-NK cells. FIG. 4D shows total perforin and granzyme B expression 21 days post-expansion.

FIG. 5A-5D depict daratumumab- and elotuzumab-mediated Interferon-γ expression levels of g-NK cells expanded in the presence of 221.AEH or K562-mbIL15-41BBL feeder cells with or without IL-21 included in the NK cell media. FIG. 5A shows g-NK cell Interferon-γ expression levels 13 days post-expansion against the LP1 cell line. FIG. 5B shows g-NK cell Interferon-γ expression levels 13 days post-expansion against the MM.1S cell line. FIG. 5C shows g-NK cell Interferon-γ expression levels 21 days post-expansion against the LP1 cell line. FIG. 5D shows g-NK cell Interferon-γ expression levels 21 days post-expansion against the MM.1S cell line.

FIG. 6A-6D depict daratumumab- and elotuzumab-mediated TNF-α expression levels of g-NK cells expanded in the presence of 221.AEH or K562-mbIL15-41BBL feeder cells with or without IL-21 included in the NK cell media. FIG. 6A shows g-NK cell TNF-α expression levels 13 days post-expansion against the LP1 cell line. FIG. 6B shows g-NK cell TNF-α expression levels 13 days post-expansion against the MM.1S cell line. FIG. 6C shows g-NK cell TNF-α expression levels 21 days post-expansion against the LP1 cell line. FIG. 6D shows g-NK cell TNF-α expression levels 21 days post-expansion against the MM.1S cell line.

FIG. 7 depicts g-NK cell expansion of NK cells expanded for 15 days in the presence of various cytokine mixtures and concentrations.

FIG. 8A-8J show cell effector function of g-NK cells expanded in the presence of various cytokine mixtures and concentrations.

FIG. 8A and FIG. 8B depict daratumumab- and elotuzumab-mediated cytotoxic activity of g-NK cells expanded in the presence of various cytokine mixtures and concentrations. FIG. 8A shows g-NK cell cytotoxicity against the LP1 cell line. FIG. 8B shows g-NK cell cytotoxicity against the MM.1S cell line.

FIG. 8C and FIG. 8D depict daratumumab- and elotuzumab-mediated degranulation levels (CD107a^{pos}) of g-NK cells expanded in the presence of various cytokine mixtures and concentrations. FIG. 8C shows g-NK cell degranulation levels against the LP1 cell line. FIG. 8D shows g-NK cell degranulation levels against the MM.1S cell line.

FIG. 8E and FIG. 8F depict levels of perforin and granzyme B expression in g-NK cells expanded in the presence of various cytokine mixtures and concentrations. FIG. 8E shows perforin and granzyme B expression as percentages of g-NK cells. FIG. 8F shows total perforin and granzyme B expression.

FIG. 8G and FIG. 8H depict daratumumab- and elotuzumab-mediated Interferon-γ expression levels of g-NK cells expanded in the presence of various cytokine mixtures and concentrations. FIG. 8G shows g-NK cell Interferon-γ expression levels against the LP1 cell line. FIG. 8H shows g-NK cell Interferon-γ expression levels against the MM.1S cell line.

FIG. 8I and FIG. 8J depict daratumumab- and elotuzumab-mediated TNF-α expression levels of g-NK cells expanded in the presence of various cytokine mixtures and concentrations. FIG. 8I shows g-NK cell TNF-α expression levels against the LP1 cell line. FIG. 8J shows g-NK cell TNF-α expression levels against the MM.1S cell line.

FIG. 9A-9L show expansion and cell effector function of g-NK cells expanded for 14 days in the presence of IL-21 compared to g-NK cells expanded without IL-21 (n=6).

FIG. 9A and FIG. 9B depict the expansion of g-NK cells expanded in the presence of IL-21 compared to g-NK cells expanded without IL-21. FIG. 9A shows g-NK cell percentage before and after expansion. FIG. 9B shows the number of g-NK cells expanded per 10 million NK cells. Values are mean±SE. #p<0.001 for comparisons of CD3^{neg}/CD57^{pos}+IL-21 expansions vs. CD3^{neg}/CD57^{pos} expansions without IL-21. \hat{ }p<0.05 for comparisons of CD3^{neg}/CD57^{pos} expansions vs. other CMV^{pos} expansions. *p<0.001 for comparisons of CMV^{pos} expansions vs. CMV^{neg} CD3^{neg} expansion.

FIG. 9C depicts comparison of the proportion of g-NK (% of total NK-cells from CMV+ (n=8) and CMV− donors (n=6) before and after expansion. FIG. 9D depicts comparison of the n-fold expansion rate of g-NK from CMV+ and CMV− donors. FIG. 9E provides representative flow plot of FcεR1γ vs. CD56 for a CMV+ donor. FIG. 9F provides representative histogram of FcεR1γ expression on CD3−/CD56+ NK-cells for CMV+ and CMV− donors. Independent samples t-tests were used to determine the differences between CMV+ and CMV− donors before and after expansion (FIG. 9C and FIG. 9D). Values are mean±SE. *p<0.05, **p<0.01, and ***p<0.001.

FIG. 9G and FIG. 9H depict daratumumab- and elotuzumab-mediated cytotoxic activity 14 days post-expansion of g-NK cells expanded in the presence of IL-21 compared to g-NK cells expanded without IL-21. FIG. 9G shows g-NK cell cytotoxicity against the LP1 cell line. FIG. 9H shows g-NK cell cytotoxicity against the MM.1S cell line. Values are mean±SE. *p<0.05, **p<0.01, and ***p<0.001 for comparisons of CD3^{neg}/CD57^{pos}+IL-21 expansions vs. CD3^{neg}/CD57^{pos} expansions without IL-21.

FIG. 9I and FIG. 9J depict daratumumab- and elotuzumab-mediated degranulation levels (CD107a^{pos}) of g-NK cells expanded in the presence of IL-21 compared to g-NK cells expanded without IL-21. FIG. 9I shows g-NK cell degranulation levels 14 days post-expansion against the LP1 cell line. FIG. 9J shows g-NK cell degranulation levels 14 days post-expansion against the MM.1S cell line. Values are mean±SE. *p<0.05, **p<0.01, and ***p<0.001 for comparisons of CD3^{neg}/CD57^{pos}+IL-21 expansions vs. CD3^{neg}/CD57^{pos} expansions without IL-21.

FIG. 9K and FIG. 9L depict levels of perforin and granzyme B expression in g-NK cells expanded in the presence of IL-21 compared to g-NK cells expanded without IL-21. FIG. 9K shows perforin and granzyme B expression 14 days post-expansion as percentages of NK cells. FIG. 9L shows total perforin and granzyme B expression 14 days post-expansion. Values are mean±SE. *p<0.05, **p<0.01, and ***p<0.001 for comparisons of CD3^{neg}/CD57^{pos}+IL-21 expansions vs. CD3^{neg}/CD57^{pos} expansions without IL-21.

FIG. 9M depicts baseline expression of perforin (left) and granzyme B (right) in expanded g-NK cells than cNK cells (n=5). To compare effector perforin and granzyme B expression between g-NK and cNK, an independent sample t-test was used. Values are mean±SE. Statistically significant differences from cNK cells are indicated by ***p<0.001.

FIG. 9N depicts representative histograms of perforin and granzyme B expression for g-NK and cNK cells.

FIG. 9O and FIG. 9P depict daratumumab- and elotuzumab-mediated Interferon-γ expression levels of g-NK cells expanded in the presence of IL-21 compared to g-NK cells expanded without IL-21. FIG. 9O shows g-NK cell Interferon-γ expression levels 14 days post-expansion against the LP1 cell line. FIG. 9P shows g-NK cell Interferon-γ expression levels 14 days post-expansion against the MM.1S cell line. Values are mean±SE. *p<0.05, **p<0.01, and ***p<0.001 for comparisons of CD3^{neg}/CD57^{pos}+IL-21 expansions vs. CD3^{neg}/CD57^{pos} expansions without IL-21.

FIG. 9Q and FIG. 9R depict daratumumab- and elotuzumab-mediated TNF-α expression levels of g-NK cells expanded in the presence of IL-21 compared to g-NK cells expanded without IL-21. FIG. 9Q shows g-NK cell TNF-α expression levels 14 days post-expansion against the LP1 cell line. FIG. 9R shows g-NK cell TNF-α expression levels 14 days post-expansion against the MM.1S cell line. Values are mean±SE. *p<0.05, **p<0.01, and ***p<0.001 for comparisons of CD3^{neg}/CD57^{pos}+IL-21 expansions vs. CD3^{neg}/CD57^{pos} expansions without IL-21.

FIG. 9S depicts daratumumab- and elotuzumab-mediated interferon-γ expression levels of expanded g-NK cells compared to cNK cells against MM.1S cell line among different donors. FIG. 9T depicts daratumumab- and elotuzumab-mediated TNF-α expression levels of expanded g-NK cells compared to cNK cells against MM.1S cell line among different donors.

FIG. 10 depicts the expansion of g-NK expanded in the presence of an IL-21/anti-IL-21 complex (n=4). Values are mean±SE. #p<0.001 for comparisons of expansions with IL-21 vs. expansions with IL-21/anti-IL-21 complex.

FIG. 11A-11H show NK cell effector function of previously cryopreserved g-NK cells compared to that of freshly enriched g-NK cells (n=4). Values are mean±SE. #p<0.05 for comparisons of freshly enriched g-NK cells vs. previously cryopreserved g-NK cells.

FIG. 11A and FIG. 11B depict daratumumab- and elotuzumab-mediated degranulation levels (CD107a^{pos}) of previously cryopreserved g-NK cells compared to freshly enriched g-NK cells. FIG. 11A shows g-NK cell degranulation levels against the LP1 cell line. FIG. 11B shows g-NK cell degranulation levels against the MM.1S cell line.

FIG. 11C and FIG. 11D depict levels of perforin and granzyme B expression in previously cryopreserved g-NK cells compared to freshly enriched g-NK cells. FIG. 11C shows total perforin expression of g-NK cells. FIG. 11D shows total granzyme B expression of g-NK cells.

FIG. 11E and FIG. 11F depict daratumumab- and elotuzumab-mediated Interferon-γ expression levels of previously cryopreserved g-NK cells compared to freshly enriched g-NK cells. FIG. 11E shows g-NK cell Interferon-γ expression levels against the LP1 cell line. FIG. 11F shows g-NK cell Interferon-γ expression levels against the MM.1S cell line.

FIG. 11G and FIG. 11H depict daratumumab- and elotuzumab-mediated TNF-α expression levels of previously cryopreserved g-NK cells compared to freshly enriched g-NK cells. FIG. 37G shows g-NK cell TNF-α expression levels against the LP1 cell line. FIG. 11H shows g-NK cell TNF-α expression levels against the MM.1S cell line.

FIGS. 12A-C depict the persistence of cNK (cryopreserved) and g-NK (cryopreserved or fresh) cells in NSG mice after infusion of a single dose of 1×10⁷ expanded cells. FIG. 12A shows the number of cNK and g-NK cells in peripheral blood collected at days 6, 16, 26, and 31 post-infusion. FIG. 12B shows the number of NK cells present in the spleen at day 31 post-infusion, the time of sacrifice. FIG. 12C shows the number of NK cells present in the bone marrow the time of sacrifice. N=3 for all 3 arms. Values are mean±SE. *p<0.05 and ***p<0.001 for comparisons of cryopreserved cNK cells and fresh or cryopreserved g-NK cells.

FIGS. 13A-13D depict the expression of CD20 (the target for rituximab), CD38 (the target for daratumumab), and SLAMF7 (the target for elotuzumab) on g-NK and cNK. FIG. 13A shows the percentage of expanded g-NK cells, unexpanded NK-cells (CD3^{neg}/CD56^{pos}), and MM.1S cells expressing CD20. FIG. 13B shows the percentage of expanded g-NK cells, unexpanded NK-cells (CD3^{neg}/CD56^{pos}), and MM.1S cells expressing CD38. FIG. 13C shows the percentage of expanded g-NK cells, unexpanded NK-cells (CD3^{neg}/CD56^{pos}), and MM.1S cells expressing SLAMF7. FIG. 13D shows the percentage of cNK and g-NK expressing CD38 before and after expansion. N=3 for all arms.

FIG. 13E depicts the mean fluorescence intensity (MFI) for CD38^{pos} NK-cells before and after expansion (n=4). FIG. 13F provides a representative histogram depicting the reduced CD38 expression of g-NK cells relative to cNK and MM.1S cells. Values are mean±SE. #p<0.001 for comparisons of g-NK cells vs. all other cells. FIG. 13G depicts comparison of daratumumab-induced fratricide by expanded g-NK and cNK cells

FIGS. 14A-F show effect of treatment with cNK and daratumumab (cNK+Dara) or g-NK and daratumumab (g-NK+Dara) on tumor burden and survival in a mouse model of multiple myeloma. 5×10⁵ luciferase-labeled MM.1S human myeloma cells were injected intravenously (I.V.) into the tail veins of female NSG mice. Weekly, for a duration of five weeks, expanded NK cells were I.V. administered (6.0×10⁶ cells per mouse) and daratumumab was I.P. injected (10 μg per mouse) to NSG mice. FIG. 14A shows BLI imaging of mice twice per week at days 20, 27, 37, 41, 48, and 57 following tumor inoculation (left). Correspondent days post-treatment are shown on the right side of the figure. Colors indicate intensity of BLI (blue, lowest; red, highest). FIG. 14B shows tumor BLI (photons/second) over time in the g-NK+Dara group relative to the control and cNK+Dara groups. *p<0.05 for comparisons of g-NK and control or cNK groups. FIG. 14C shows percent survival over time, and arrows indicate administration of therapy with either cNK+Dara or g-NK+Dara. FIG. 14D presents the change in body weight over time of mice in the control, cNK+Dara, and g-NK+Dara groups. FIG. 14E depicts the number of CD138⁺ tumor cells present in bone marrow at the time of sacrifice in cNK+Dara- and g-NK+Dara-treated mice. *** p<0.001 for comparisons of g-NK and cNK cells. Values are mean±SE.

FIG. 14F shows a representative flow plot using a gating strategy to resolve the presence of NK cells and tumor cells in the control group and in mice treated with either cNK+Dara or g-NK+Dara. N=8 for the control group, and N=7 for the g-NK or cNK group.

FIG. 14G presents all BLI images collected over the entire study for all control, cNK+Dara, and g-NK+Dara treated mice. Colors indicate intensity of BLI (blue, lowest; red, highest). FIG. 14H depicts X-ray images obtained for all mice in the control, cNK+Dara, and g-NK+Dara groups prior to sacrifice. Arrows indicate bone fractures and deformities. The day of sacrifice is indicated under each mouse.

FIGS. 15A-C present comparative data of persistent NK cells in NSG mice following treatment with cNK+Dara or g-NK+Dara. All data present the amount of cells detected using flow cytometry at the time of sacrifice. FIG. 15A shows the number of cNK and g-NK cells in blood. FIG. 15B shows the number of NK cells present in the spleen. FIG. 15C shows the number of NK cells present in bone marrow. Values are mean±SE. *** p<0.001 for comparisons of g-NK and cNK cells.

FIG. 16 depicts the percentage of g-NK (CD45^{pos}/CD3^{neg}/CD56^{pos}/FcRγ^{neg}) within a cell subset having either the surrogate extracellular surface phenotype of CD45^{pos}/CD3^{neg}/CD56^{pos}/CD16^{pos}/CD57^{pos}/CD7^{dim/neg}/CD161^{neg} or CD45^{pos}/CD3^{neg}/CD56^{pos}/NKG2A^{neg}/CD161^{neg}. Values are mean standard error.

FIG. 17 depicts the post-transduction expression of GFP and CD20-CAR by g-NK cells in two separate experiments, each using a distinct donor.

FIG. 18 depicts the potency of the g-NK cells with or without a CD20-CAR, in the presence or absence or rituximab (anti-CD20 monoclonal antibody) against Raji lymphoma cells.

### DETAILED DESCRIPTION

Provided herein is an engineered Natural Killer (NK) cell deficient in expression of FcRγ chain (g-NK cells), further comprising a recombinant chimeric antigen receptor (CAR) and compositions thereof. FcRγ is also known as FcεR1γ, which is used interchangeably herein. Also provided herein are methods of producing genetically engineered g-NK cells comprising introducing into a g-NK cell a nucleic acid encoding a CAR, thereby producing a genetically engineered g-NK cell. In some embodiments, methods of engineering the CAR results in transient expression of the CAR in the engineered NK cells. In some embodiments, methods of engineering the CAR results in stable expression of the CAR in the engineered NK cells.

Provided herein is an engineered Natural Killer (NK) cell deficient in expression of FcRγ chain (g-NK cells), further comprising a heterologous nucleic acid encoding an immunomodulator and compositions thereof. In some embodiments, the immunomodulator may be any agent that may increase or improve activity of the NK cells. In some embodiments, the immunomodulator is an exogenous cytokine, such as IL-15. Also provided herein are methods of producing genetically engineered g-NK cells comprising introducing into the g-NK cell a nucleic acid encoding an immunomodulator, thereby producing a genetically engineered g-NK cell. In some embodiments, methods of engineering the immunomodulator (e.g. cytokine, such as IL-15) results in transient expression of the immunomodulator in the engineered NK cells. In some embodiments, methods of engineering the immunomodulator (e.g. cytokine, such as IL-15) results in stable expression of the immunomodulator in the engineered NK cells.

Provided herein is an engineered Natural Killer (NK) cell deficient in expression of FcRγ chain (g-NK cells), further comprising a recombinant chimeric antigen receptor (CAR) and a heterologous nucleic acid encoding an immunomodulator and compositions thereof. Also provided herein are methods of producing genetically engineered g-NK cells comprising (a) introducing into a g-NK cell a nucleic acid encoding a CAR, and (b) introducing into the g-NK cell a nucleic acid encoding an immunomodulator, thereby producing a genetically engineered g-NK cell, wherein the introducing a nucleic acid encoding a CAR and the introducing a nucleic acid encoding an immunomodulator can be carried our simultaneously or sequentially in any order. In some embodiments, methods of engineering the CAR and the immunomodulator (e.g. cytokine, such as IL-15) results in transient expression of the CAR and/or immunomodulator in the engineered NK cells. In some embodiments, methods of engineering the CAR and the immunomodulator (e.g. cytokine, such as IL-15) results in stable expression of the CAR and immunomodulator in the engineered NK cells. In some embodiments, one of the CAR or immunomodulatory is transiently expressed in the engineered cells and the other of the CAR and immunomodulatory is stably expressed.

Natural killer (NK) cells are innate lymphocytes important for mediating anti-viral and anti-cancer immunity through cytokine and chemokine secretion, and through the release of cytotoxic granules (Vivier et al. Science 331(6013):44-49 (2011); Caligiuri, Blood 112(3):461-469 (2008); Roda et al., Cancer Res. 66(1):517-526 (2006)). NK cells are effector cells that comprise the third largest population of lymphocytes and are important for host immuno-surveillance against tumor and pathogen-infected cells. However, unlike T and B lymphocytes, NK cells use germline-encoded activation receptors and are thought to have only a limited capacity for target recognition (Bottino et al., Curr Top Microbiol Immunol. 298:175-182 (2006); Stewart et al., Curr Top Microbiol Immunol. 298:1-21 (2006)).

Activation of NK cells can occur through the direct binding of NK cell receptors to ligands on the target cell, as seen with direct tumor cell killing, or through the crosslinking of the Fc receptor (CD16; also known as CD16a or FcγRIIIa) by binding to the Fc portion of antibodies bound to an antigen-bearing cell. Upon activation, NK cells produce cytokines and chemokines abundantly and at the same time exhibit potent cytolytic activity. NK cells are capable of killing tumor cells via antibody dependent cell mediated cytotoxicity (ADCC). In some cases, ADCC is triggered when receptors on the NK cell surface (such as CD16) recognize IgG1 or IgG3 antibodies bound to the surface of a cell. This triggers release of cytoplasmic granules containing perforin and granzymes, leading to target cell death. Because NK cells express the activating Fc receptor CD16, which recognizes IgG-coated target cells, target recognition is broadened (Ravetch & Bolland, Annu Rev Immunol. 19:275-290 (2001); Lanier Nat. Immunol. 9(5):495-502 (2008); Bryceson & Long, Curr Opin Immunol. 20(3):344-352 (2008)). ADCC and antibody-dependent cytokine/chemokine production are primarily mediated by NK cells.

CD16 also exists in a glycosylphosphatidylinositol-anchored form (also known as FcγRIIIB or CD16B). It is understood that reference to CD16 herein is with reference to the CD16a form that is expressed on NK cells and that is involved in antibody-dependent responses (such as NK cell-mediated ADCC), and it is not meant to refer to the glycosylphosphatidylinositol-anchored form.

The CD16 receptor is able to associate with adaptors, the chain of the TCR-CD3 complex (CD3ζ) and/or the FcRγ chain, to transduce signals through immunoreceptor tyrosine-based activation motifs (ITAMs). In some aspects, CD16 engagement (CD16 crosslinking) initiates NK cell responses via intracellular signals that are generated through one, or both, of the CD16-associated adaptor chains, FcRγ or CD3ζ. Triggering of CD16 leads to phosphorylation of the γ or ζ chain, which in turn recruits tyrosine kinases, syk and ZAP-70, initiating a cascade of signal transduction leading to rapid and potent effector functions. The most well-known effector function is the release of cytoplasmic granules carrying toxic proteins to kill nearby target cells through the process of antibody-dependent cellular cytotoxicity. CD16 crosslinking also results in the production of cytokines and chemokines that, in turn, activate and orchestrate a series of immune responses.

This release of cytokines and chemokines can play a role in the anti-cancer activity of NK cells in vivo. NK cells also have small granules in their cytoplasm containing perforin and proteases (granzymes). Upon release from the NK cell, perforin forms pores in the cell membrane of targeted cells through which the granzymes and associated molecules can enter, inducing apoptosis. The fact that NK cells induce apoptosis rather than necrosis of target cells is significant—necrosis of a virus-infected cell would release the virions, whereas apoptosis leads to destruction of the virus inside the cells.

A specialized subset of NK cells lacking the FcRγ adaptor protein, also known as g-NK cells, are able to mediate robust ADCC responses (see e.g. published Patent Appl. No. US2013/0295044). The mechanism for increased responses may be due to changes in epigenetic modification that influence the expression of the FcRγ. The g-NK cells express the signaling adaptor ζ chain abundantly, but are deficient in the expression of the signaling adaptor γ chain. Compared to conventional NK cells, these γ-deficient g-NK cells exhibit dramatically enhanced activity when activated by antibodies. For example, the g-NK cells can be activated by antibody-mediated crosslinking of CD16 or by antibody-coated tumor cells. In some aspects, the g-NK cells produce greater amounts of cytokines (e.g. IFN-γ or TNF-α) and chemokines (e.g. MIP-1α, MIP-1β, and RANTES) and/or display higher degranulation responses than conventional NK cells expressing the γ chain. The g-NK cells provide high expression of Granzyme B, a component of natural killer cell cytotoxic machinery. Moreover, the g-NK cells have a prolonged lifespan, compared to conventional NK cells, and their presence is maintained long-term. In some embodiments, g-NK cells are functionally and phenotypically stable.

In some embodiments, g-NK cells are more effective in eliciting ADCC responses than conventional NK cells, e.g. NK cells that are not deficient in the γ chain. In some embodiments, g-NK cells are more effective in eliciting cell-mediated cytotoxicity than are conventional NK cells even in the absence of antibody. In some cases, ADCC is a mechanism of action of therapeutic antibodies, including anti-cancer antibodies. In some aspects, cell therapy by administering NK cells can be used in concert with antibodies for therapeutic and related purposes.

For instance, certain therapeutic monoclonal antibodies, such as daratumumab targeting CD38 and elotuzumab targeting SLAMF7 are FDA approved for treating disease, such as multiple myeloma (MM). While clinical responses of therapeutic antibodies are promising, they are often not ideal. For example, while initial clinical responses have generally been encouraging, particularly for daratumumab, essentially all patients eventually develop progressive disease. Thus, there is a significant need for new strategies to either drive deeper remissions or overcome resistance to these agents. The provided embodiments, including compositions, address these needs.

Provided herein is an engineered Natural Killer (NK) cell deficient in expression of FcRγ chain (g-NK cells), further comprising a recombinant chimeric antigen receptor (CAR) and compositions containing the same. Provided herein is an engineered Natural Killer (NK) cell deficient in expression of FcRγ chain (g-NK cells), further comprising a heterologous nucleic acid encoding an immunomodulator and compositions thereof. Provided herein is an engineered Natural Killer (NK) cell deficient in expression of FcRγ chain (g-NK cells), further comprising a recombinant chimeric antigen receptor (CAR) and a heterologous nucleic acid encoding an immunomodulator and compositions containing the same. Further provided are methods of engineering the g-NK cells. In some embodiments, CAR-dependent antigen targeting by engineering of or engineered g-NK cells leads to improved outcomes for patients due to the improved affinity, cytotoxic and/or cytokine-mediated effect functions of the g-NK cell subset. In some embodiments, the engineered g-NK cells are administered to the subject in combination with a therapeutic antibody directed at a tumor or other pathogen or disease antigen. In some embodiments, antibody-directed targeting of g-NK cells leads to improved outcomes for patients due to the improved affinity, cytotoxic and/or cytokine-mediated effect functions of the g-NK cell subset.

Although NK-cells are normally activated when the Fc portion of an antibody binds their Fc receptor (FcγRIIIa or CD16a) and triggers activation and degranulation through a process involving the adapter proteins CD3ζ and FcεR1γ, such methods involve separate administration of an antibody against a desired target and exposure of the NK cells to the antibody. Further, binding and crosslinking of the Fc receptor CD16 on conventional NK cells engages signaling via both the CD3ζ and FcεR1γ, which can lead to variability in signaling depending on the expression of the signaling adaptors in the NK cells. Finally, activity of NK cell activity often requires cytokine support, such as by IL-15, to boost cytotoxic activity; thus, absence of sufficient supporting cytokines may limit durability of the response. Each of the above factors, alone and together, has hampered the utility of certain NK cell therapies.

The engineered NK cells and compositions containing the same provided herein, such as produced by the provided methods, offer an improved cell therapy in several respects First, the provided g-NK cells and compositions containing the same, such as produced by the provided methods, are engineered to express a chimeric antigen receptor (CAR), an immunomodulator such as a co-stimulatory cytokine, or a chimeric antigen receptor (CAR) and an immunomodulator such as a co-stimulatory cytokine. Expression of the CAR enables the g-NK cells to be administered without a separate monoclonal antibody required for targeting the g-NK cells to the target cells or tissue in an affected subject or individual. Further, the ability of the NK cells to produce and secrete an exogenous immunomodulator, such as a cytokine, means that the NK cells have a built in support to boost activity. Finally, the provided NK cells compositions are enriched in g-NK cells (i.e. NK cells deficient in FcεR1γ), which offer a number of advantages compared to conventional NK cells or NK cells enriched in other subsets. G-NK cells are a relatively rare subset as g-NK cells are only detectable at levels of ˜3-10% of total NK-cells in only 25-30% of CMV seropositive individuals. The provided methods relate to methods that are particularly robust in the ability to expand and enrich g-NK cells, thus allowing sufficient expansion required for in vivo use, while also being amenable to engineering of the enriched g-NK cells with the CAR, the immunomodulator (e.g. cytokine, such as IL-15), or the CAR and the immunomodulator (e.g. cytokine, such as IL-15) prior to, during or subsequent to their expansion. In some embodiments, the engineered g-NK cells are administered to a subject in combination with a monoclonal antibody for targeting the g-NK cells to the target cells or tissue in an affected subject or individual. The provided cells and compositions produced by such methods are particularly robust in their ability to target the g-NK cells to the appropriate location in a subject or individual.

g-NK cells represent a relatively small percentage of NK cells in the peripheral blood, thereby limiting the ability to use these cells in therapeutic methods. In particular, to utilize g-NK cells in the clinic, a high preferential expansion rate is necessary because g-NK cells are generally a rare population. Other methods for expanding NK cells are able to achieve thousand-fold 14-day NK-cell expansion rates, but they yield low differentiation, NKG2C^{neg}, FcεR1γ^{pos} (FcRγ^{pos}) NK-cells (Fujisaki et al. (2009) Cancer Res., 69:4010-4017; Shah et al. (2013) PLoS One, 8:e76781). Further, it is found herein that an expansion optimized for expanding NK cells that phenotypically overlap with g-NK cells does not preferentially expand g-NK cells to amounts that would support therapeutic use. In particular, it has been previously reported that NKG2C^{pos} NK-cells, which exhibit phenotypic overlap with g-NK cells, can be preferentially expanded using HLA-E transfected 221.AEH cells and the inclusion of IL-15 in the culture medium (Bigley et al. (2016) Clin. Exp. Immunol., 185:239-251). Culture with such HLA-expressing cells that constitutively expresses HLA-E pushes the NK-cells in the direction of an NKG2C^{pos}/NKG2A^{neg} phenotype (NKG2C is the activating receptor for HLA-E, while NKG2A is the inhibitory receptor for HLA-E). It was thought that because such cells include within it the g-NK, such methods would be sufficient to expand g-NK cells. However, this method does not achieve robust expansion of g-NK cells.

Methods described herein are able to produce NK cell compositions enriched in g-NK cells that overcome these limitations. The provided methods utilize a greater ratio of HLA-E+ feeder cells deficient in HLA class I and HLA class II, for instance 221.AEH cells, to NK-cells compared to previous methods. In particular, previous methods have used a lower ratio of 221.AEH cells, such as a ratio of 10:1 NK cell to 221.AEH ratio. It is found herein that a greater ratio of HLA-E-expressing feeder cells, such as 221.AEH cells, results in overall expansion that is greater and more skewed towards the g-NK phenotype. In some embodiments, the greater ratio of HLA-E+ feeder cells, for instance 221.AEH cells, is possible by irradiating the feeder cells. In some aspects, the use of irradiated feeder cell lines also is advantageous because it provides for a method that is GMP compatible. The inclusion of any of recombinant IL-2, IL-7, IL-15, IL-12, IL-18, IL-21, IL-27, or combinations thereof during the expansion also is found to support robust expansion. In particular embodiments of the provided methods at least one recombinant cytokine is IL-2. In some embodiments, there are two or more recombinant cytokines wherein at least one recombinant cytokine is IL-2 and at least one recombinant cytokine is IL-21.

Provided methods herein are based on the finding that culture of NK cells for expansion in the presence of IL-21 supercharges the NK cells to produce cytokines or effector molecules such as perforin and granzyme B. Compositions containing NK cells produced by the expanded processes herein are highly functional, exhibit robust proliferation, and work well even after they are cryofrozen without rescue. For example, the NK cells produced by the provided processes when expanded in the presence of IL-21 not only exhibit strong ADCC activity, but they also exhibit antibody-independent cytotoxic activities. The robust activity, including antibody-independent cytotoxic activities, are particularly suitable for strategies as described herein in which cells are further engineered with a CAR, an immunomodulatory, or a CAR and an immunomodulator, as the NK cells are primed and ready for effector activity after engagement of the CAR by a target antigen or an Fc receptor CD16 by a co-administered antibody that has recognized a target antigen. For example, effector molecules (e.g. perforin and granzymes) are spontaneously present in NK cells expanded by the provided methods, thereby providing cells that exhibit high cytotoxic potential. As shown herein, NK cell composition produced by the provided processes that include IL-21 (e.g. IL-2, IL-15 and IL-21) not only exhibit a higher percentage of NK cells positive for perforin or granzyme B than NK cell compositions produced by a process that only includes IL-2 without addition of IL-21, but they also exhibit a higher average level or degree of expression of the molecules in the cells. Further, the NK cell composition produced by the method provided herein that includes IL-21 (e.g. IL-2, IL-15 and IL-12) also result in g-NK cell compositions that exhibit substantial effector activity, including degranulation and ability to express more IFN-gamma and TNF-alpha, in response to target cells. This functional activity is highly preserved even after cryopreservation and thawing of expanded NK cells. The marked increases in cytolytic enzymes, as well as more robust activation phenotypes, underpin the enhanced capacity of expanded g-NK cells to induce apoptosis of tumor targets. These activities are exemplified in examples herein upon engagement via CD16 crosslinking with antibody directed against a target antigen. While many of these activities are exemplified in examples herein upon engagement with antibody via CD16 crosslinking, similar activities result upon engagement of the CAR with target antigen as signaling is also mediated via CD3ζ. The marked antibody-independent effector phenotype, coupled with the engineering of the cells with a CAR, an immunomodulator (e.g. cytokine) or a CAR with an immunomodulator (e.g. cytokine), also supports potential utility of the g-NK cells as a monotherapy.

Further, in some embodiments, the g-NK cells produce significantly greater amounts of a cytokine than natural killer cells that do express FcRγ. In another embodiment, the cytokine is interferon-gamma (IFN-γ), tumor necrosis factor-α (TNF-α), or a combination thereof. In one embodiment, the g⁻NK cells produce significantly greater amounts of a chemokine. In one embodiment, the chemokine is MIP-1α, MIP-1β or a combination thereof. In another embodiment, the g⁻NK cells produce the cytokine or the chemokine upon signaling via CD3ζ, such as may occur via engagement of the CAR or, in some cases, stimulation through the Fc receptor CD16.

Further, findings herein also demonstrate the potential of the provided NK cells expanded in the presence of IL-21 to persist and proliferate well for an extended period of time, which is greater than cells expanded, for example, only in the presence of IL-2 without the addition of IL-21. Furthermore, results showed that cryopreserved g-NK cells persisted at comparable levels to fresh g-NK cells. This significantly improved persistence emphasizes the potential utility of fresh or cryopreserved g-NK as an off-the-shelf cellular therapy to enhance target-directed cytotoxicity or antibody-mediated ADCC. This finding of improved persistence is advantageous, since clinical utility of many NK cell therapies has been hampered by limited NK cell persistence.

It also is found that enrichment of NK cells from a cell sample prior to the expansion method, such as by enrichment for CD16 or CD57 cells prior to expansion, further substantially increases the amount of g-NK cell expansion that can be achieved compared to methods that initially enrich NK cells based on CD3 depletion alone. In another embodiment, another enrichment that can be carried out prior to expansion is enriching for NK cells by positive selection for CD56 and negative selection or depletion for CD38. In a further embodiment, another enrichment that can be carried out prior to expansion is enriching for NK cells by positive selection for CD56 followed by negative selection or depletion for NKG2A^{neg} and negative selection or depletion for CD161^{neg}. In another embodiment, another enrichment that can be carried out prior to expansion is enriching for NK cells by positive selection for CD57 followed by negative selection or depletion for NKG2A and/or positive selection for NKG2C. In another embodiment, another enrichment that can be carried out prior to expansion is enriching for NK cells by positive selection for CD56 followed by negative selection or depletion for NKG2A and/or positive selection for NKG2C. In any of such embodiments, enrichment for NKG2C^{pos} and/or NKG2A^{neg} NK cells can be carried out after expansion.

In any of such embodiments, the enriched NK cells can be enriched from a cell sample containing NK cells, such as from peripheral blood mononuclear cells (PBMCs). In some embodiments, prior to the enrichment for NK cells from the cell sample, T cells can be removed by negative selection or depletion for CD3. In any of such embodiments, the enriched NK cells can be enriched from a biological sample from a human subject containing NK cells (e.g. PBMCs) with a relatively high proportion of g-NK cells, for instance from a human subject selected for having a high percentage of g-NK cells among NK cells. In any of such embodiments, the enriched NK cells can be enriched from a biological sample from a human subject containing NK cells, e.g. PBMCs, in which the sample contains a relatively high proportion of NKG2C^{pos} NK cells (e.g. at or about or greater than 20% NKG2C^{pos} NK cells) and/or NKG2A^{neg} NK cells (e.g. at or about or greater than 70% NKG2A^{neg} NK cells). In any of such embodiments, the enriched NK cells can be enriched from a biological sample from a human subject containing NK cells, e.g. PBMCs, in which the sample contains a relatively high proportion of NKG2C^{pos} NK cells (e.g. at or about or greater than 20% NKG2C^{pos} NK cells) and NKG2A^{neg} NK cells (e.g. at or about or greater than 70% NKG2A^{neg} NK cells). In particular embodiments, the subject in which the sample is from is CMV seropositive, as such subjects have greater detectable g-NK cells in their peripheral blood.

Together, the provided approach for expanding g-NK cells can achieve expansion of NK cells that exceeds over 1 billion cells, and in some cases up to 8 billion or more, from an initial 10 million enriched NK cells at the initiation of culture. In particular, the provided methods can result in high-yield (>1000 fold) expansion rates with maintained or, in some cases, increased functionality of the g-NK cells after expansion. In some embodiments, the provided methods can result in a g-NK cell population expressing high levels of perforin and granzyme B. Further, it is found that the provided methods are sufficient to expand previously frozen NK cells, which is not commonly achieved by many existing methods that involve rescue of thawed NK cells. In some embodiments, this is achieved by increasing the duration of the expansion protocol. In some embodiments, this is achieved by decreasing the ratio of HLA-E+ feeder cells to NK cells, e.g. to about 1:1 221.AEH to NK cells. In some embodiments, this is achieved with the inclusion of any of recombinant IL-2, IL-7, IL-15, IL-12, IL-18, IL-21, IL-27, or combinations thereof during the expansion. In particular embodiments, at least one recombinant cytokine is IL-2. In some embodiments, expansion is carried out in the presence of two or more recombinant cytokines in which at least one is recombinant IL-21 and at least one is recombinant IL-2.

As shown here, the provided engineered g-NK cells and compositions containing the same, such produced by the provided methods, can be used for cancer therapy. In some embodiments, adoptive transfer of the NK-cells does not result in severe graft-versus-host (GVHD), and thus such a cell therapy, including in combination with an antibody as an antibody-directed NK-cell therapy, can be given in an “off-the-shelf” manner for clinical use. In some aspects, the NK cells may be further engineered to reduce or eliminate individual HLA molecules in the NK cells, thereby improving allogeneic potential of the provided cell therapy.

All references cited herein, including patent applications, patent publications, and scientific literature and databases, are herein incorporated by reference in their entirety for all purposes to the same extent as if each individual reference were specifically and individually indicated to be incorporated by reference.

For clarity of disclosure, and not by way of limitation, the detailed description is divided into the subsections that follow. The section headings used herein are for organizational purposes only and are not to be construed as limiting the subject matter described.

### I. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

As used herein, “antibody” refers to immunoglobulins and immunoglobulin fragments, whether natural or partially or wholly synthetically, such as recombinantly, produced, including any fragment thereof containing at least a portion of the variable heavy chain and/or light chain region of the immunoglobulin molecule that is sufficient to form an antigen binding site and, when assembled, to specifically bind antigen. Hence, an antibody includes any protein having a binding domain that is homologous or substantially homologous to an immunoglobulin antigen-binding domain (antibody combining site). Typically, antibodies minimally include all or at least a portion of the variable heavy (V_{H}) chain and/or the variable light (V_{L}) chain. In general, the pairing of a V_{H} and V_{L} together form the antigen-binding site, although, in some cases, a single V_{H} or V_{L} domain is sufficient for antigen-binding. The antibody also can include all or a portion of the constant region. Reference to an antibody herein includes full-length antibody and antigen-binding fragments. The term “immunoglobulin” (Ig) is used interchangeably with “antibody” herein.

The terms “full-length antibody,” “intact antibody” or “whole antibody” are used interchangeably to refer to an antibody in its substantially intact form, as opposed to an antibody fragment. A full-length antibody is an antibody typically having two full-length heavy chains (e.g., VH-CH1-CH2-CH3 or VH-CH1-CH2-CH3-CH4) and two full-length light chains (VL-CL) and hinge regions, such as antibodies produced from mammalian species (e.g. human, mouse, rat, rabbit, non-human primate, etc.) by antibody secreting B cells and antibodies with the same domains that are produced synthetically. Specifically whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, the antigen binding and/or the variable region of the intact antibody. Antibody fragments, include, but are not limited to, Fab fragments, Fab′ fragments, F(ab′)₂ fragments, Fv fragments, disulfide-linked Fvs (dsFv), Fd fragments, Fd′ fragments; diabodies; linear antibodies (see U.S. Pat. No. 5,641,870, Example 2; Zapata et al., Protein Eng. 8(10): 1057-1062 [1995]); single-chain antibody molecules, including single-chain Fvs (scFv) or single-chain Fabs (scFab); antigen-binding fragments of any of the above and multispecific antibodies from antibody fragments. For purposes herein, an antibody fragment typically includes one that is sufficient to engage or crosslink CD16 on the surface of an NK cell.

The term “autologous” refers to cells or tissues originating within or taken from an individual's own tissues. For example, in an autologous transfer or transplantation of NK cells, the donor and recipient are the same person.

The term “allogeneic” refers to cells or tissues that belong to or are obtained from the same species but that are genetically different, and which, in some cases, are therefore immunologically incompatible. Typically, the term “allogeneic” is used to define cells that are transplanted from a donor to a recipient of the same species.

The term “enriched” with reference to a cell composition refers to a composition in which there is an increase in the number or percentage of the cell type or population as compared to the number or percentage of the cell type in a starting composition of the same volume, such as a starting composition directly obtained or isolated from a subject. The term does not require complete removal of other cells, cell type, or populations from the composition and does not require that the cells so enriched be present at or even near 100% in the enriched composition.

The term “expression” refers to the process by which a polynucleotide is transcribed from a DNA template (such as into an mRNA or other RNA transcript) and/or the process by which a transcribed mRNA is subsequently translated into peptide, polypeptides or proteins. Transcripts and encoded polypeptides may be collectively referred to as “gene product.” If the polynucleotide is derived from genomic DNA, expression may include splicing of the mRNA in a eukaryotic cell.

The term “heterologous” with reference to a protein or nucleic acid refers to a protein or nucleic acid that has been transformed or introduced into a cell. In some cases, the heterologous protein or nucleic acid is exogenous to the cell, for example because it originates from an organism or individual other than the cell in which it is expressed. It is understood that reference to “heterologous” does not preclude that the protein or nucleic acid also may be expressed naturally by the cell into which it is introduced. A heterologous nucleic acid or encoded protein may be introduced into an NK cell, for example, by any of a variety of methods that are able to introduce or transform a nucleic acid (e.g. encoding the heterologous protein) into a cell, including viral-based methods such as by transduction or non-viral delivery methods such as electroporation or lipid nanoparticle delivery. An NK cell that has been introduced or transformed may carry the exogenous or heterologous nucleic acid extra-chromosomally or integrated in the chromosome. Integration into a cell genome and self-replicating vectors generally result in genetically stable inheritance of the transformed nucleic acid molecule. NK cells containing the transformed nucleic acids are referred to as “genetically engineered” but may also interchangeably be referred to as “recombinant” or “transformed”.

As used herein, the term “introducing” encompasses a variety of methods of introducing DNA into a cell, either in vitro or in vivo, such methods including transformation, transduction, transfection (e.g. electroporation), lipid delivery and infection. Vectors are useful for introducing DNA encoding molecules into cells. Possible vectors include plasmid vectors and viral vectors. Viral vectors include retroviral vectors, lentiviral vectors, or other vectors such as adenoviral vectors or adeno-associated vectors. Lipid nanoparticles also may be used for introducing nucleic acid, either DNA or mRNA, into cells.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides, nucleotide sequences, nucleic acids etc. can be chimeric mixtures or derivatives or modified versions thereof, single-stranded or double-stranded. They can be modified at the base moiety, sugar moiety, or phosphate backbone, for example, to improve stability of the molecule, its hybridization parameters, etc. A nucleotide sequence typically carries genetic information, including, but not limited to, the information used by cellular machinery to make proteins and enzymes. These terms include double- or single-stranded genomic DNA, RNA, any synthetic and genetically manipulated polynucleotide, and both sense and antisense polynucleotides. These terms also include nucleic acids containing modified bases.

The terms “protein,” “peptide” and “polypeptide” are used interchangeably to refer to a sequential chain of amino acids linked together via peptide bonds. The terms include individual proteins, groups or complexes of proteins that associate together, as well as fragments or portions, variants, derivatives and analogs of such proteins. Peptide sequences are presented herein using conventional notation, beginning with the amino or N-terminus on the left, and proceeding to the carboxyl or C-terminus on the right. Standard one-letter or three-letter abbreviations can be used.

The term “endogenous,” as used herein in the context of nucleic acids (e.g., genes, protein-encoding genomic regions, promoters), refers to a native nucleic acid or protein in its natural location, e.g., within the genome of a cell. In contrast, the term “exogenous,” as used herein in the context of nucleic acids, e.g., expression constructs, cDNAs, indels, and nucleic acid vectors, refers to nucleic acids that have artificially been introduced into the genome of a cell using, for example, by genetic engineering techniques such as transformation of heterologous nucleic acids or gene-editing, e.g., CRISPR-based editing techniques.

The term “composition” refers to any mixture of two or more products, substances, or compounds, including cells or antibodies. It may be a solution, a suspension, liquid, powder, a paste, aqueous, non-aqueous or any combination thereof. The preparation is generally in such form as to permit the biological activity of the active ingredient (e.g. antibody) to be effective.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, combination refers to any association between or among two or more items. The combination can be two or more separate items, such as two compositions or two collections, can be a mixture thereof, such as a single mixture of the two or more items, or any variation thereof. The elements of a combination are generally functionally associated or related.

As used herein, a kit is a packaged combination that optionally includes other elements, such as additional agents and instructions for use of the combination or elements thereof, for a purpose including, but not limited to, therapeutic uses.

As used herein, the term “treatment” or “treating” refers to clinical intervention designed to alter the natural course of the individual or cell being treated during the course of clinical pathology. Desirable effects of treatment include decreasing the rate of disease progression, ameliorating or palliating the disease state, and remission or improved prognosis. An individual is successfully “treated”, for example, if one or more symptoms associated with a disorder (e.g., an eosinophil-mediated disease) are mitigated or eliminated. For example, an individual is successfully “treated” if treatment results in increasing the quality of life of those suffering from a disease, decreasing the dose of other medications required for treating the disease, reducing the frequency of recurrence of the disease, lessening severity of the disease, delaying the development or progression of the disease, and/or prolonging survival of individuals.

An “effective amount” refers to at least an amount effective, at dosages and for periods of time necessary, to achieve the desired or indicated effect, including a therapeutic or prophylactic result. An effective amount can be provided in one or more administrations. A “therapeutically effective amount” is at least the minimum dose of cells required to effect a measurable improvement of a particular disorder. In some embodiments, a therapeutically effective amount is the amount of a composition that reduces the severity, the duration and/or the symptoms associated with cancer, viral infection, microbial infection, or septic shock in an animal. A therapeutically effective amount herein may vary according to factors such as the disease state, age, sex, and weight of the patient. A therapeutically effective amount may also be one in which any toxic or detrimental effects of the antibody are outweighed by the therapeutically beneficial effects. A “prophylactically effective amount” refers to an amount effective, at the dosages and for periods of time necessary, to achieve the desired prophylactic result. Typically but not necessarily, since a prophylactic dose is used in subjects prior to or at the earlier stage of disease, the prophylactically effective amount can be less than the therapeutically effective amount.

As used herein, an “individual” or a “subject” is a mammal. A “mammal” for purposes of treatment includes humans, domestic and farm animals, and zoo, sports, or pet animals, such as dogs, horses, rabbits, cattle, pigs, hamsters, gerbils, mice, ferrets, rats, cats, etc. In some embodiments, the individual or subject is human.

### II. ENGINEERED FC RECEPTOR GAMMA DEFICIENT NATURAL KILLER CELLS (G-NK CELLS)

Provided herein is an engineered natural killer (NK) cell deficient in expression of FcRγ (g-NK cells) that expresses a heterologous agent, such as an antigen receptor, for example a chimeric antigen receptor (CAR), that has been introduced into the g-NK cell.

Provided herein is an engineered natural killer (NK) cell deficient in expression of FcRγ (g-NK cells) that expresses a heterologous agent that has been introduced into the g-NK cells, including an immunomodulator, such as a cytokine.

Provided herein is an engineered natural killer (NK) cell deficient in expression of FcRγ (g-NK cells) that expresses heterologous agent(s) that have been introduced into the g-NK cells, including an antigen receptor, such as a chimeric antigen receptor (CAR), and an immunomodulator, such as a cytokine.

In some embodiments, the engineered NK cell is a g-NK cell deficient in expression of FcRγ. In some embodiments, the g-NK cell subset of NK cells can be detected by observing whether FcRγ is expressed by the NK cell or a population of NK cells, in which absence of FcRγ the cell is g-NK. FcRγ protein is an intracellular protein. Thus, in some aspects, the presence or absence of FcRγ can be detected after treatment of cells, for example, by fixation and permeabilization, to allow intracellular proteins to be detected.

In some cases, g-NK cells also may be identified by surface markers that are surrogate markers of g-NK cells. As described further below, it is also found that certain combinations of cell surface markers correlate with the g-NK cell phenotype, i.e. cells that lack or are deficient in intracellular expression of FcRγ, thereby providing a surrogate marker profile to identify or detect g-NK cells in a manner that does not injure the cells. In some embodiments, a surrogate marker profile for g-NK cells provided herein is based on positive surface expression of one or more markers CD16 (CD16^{pos}), NKG2C (NKG2C^{pos}), or CD57 (CD57pos) and/or based on low or negative surface expression of one or more markers CD7 (CD7^{dim/neg}), CD161 (CD161^{neg}) and/or NKG2A (NKG2A^{neg}). In some embodiments, cells are further assessed for one or more surface markers of NK cells, such as CD45, CD3 and/or CD56. In some embodiments, g-NK cells can be identified, detected, enriched and/or isolated with the surrogate marker profile CD45^{pos}/CD3^{neg}/CD56^{pos}/CD16^{pos}/CD57^{pos}/CD7^{dim/neg}/CD161^{neg}. In some embodiments, g-NK cells are identified, detected, enriched and/or isolated with the surrogate marker profile CD45^{pos}/CD3^{neg}/CD56^{pos}/NKG2A^{neg}/CD161^{neg}. In some embodiments, g-NK cells that are NKG2C^{pos} and/or NKG2A^{neg} are identified, detected, enriched for, and/or isolated.

In some embodiments, the g-NK cell has a surface phenotype that is CD16^{pos}/CD57^{pos}/CD7^{dim/neg}/CD161^{neg}. In some embodiments, the g-NK cell further has a surface phenotype that is NKG2A^{neg}/CD161^{neg}. In some embodiments, the g-NK cell further has a surface phenotype that is CD38^{neg}. In some embodiments, the g-NK cell has a surface phenotype that further is CD45^{pos}/CD3^{neg}/CD56^{pos}.

In some embodiments, the g-NK cells are engineered to express a CAR. In some embodiments, the g-NK cells are engineered to express an immunomodulator (e.g. exogenous cytokine). In some embodiments, the g-NK cells are engineered to express a CAR and an immunomodulator (e.g. exogenous cytokine). In some embodiments, the CAR is a fusion protein generally including an ectodomain that comprises an antigen recognition region, a transmembrane domain, and an endo-domain. The ectodomain (i.e., the antigen recognition region or antigen binding domain) and the transmembrane domain may be linked by a flexible linker. The endo-domain may comprise an intracellular signaling domain that propagates the external cellular stimulus intracellularly. In some embodiments, the CAR comprises 1) an antigen binding domain; 2) a flexible linker; 3) a transmembrane region; and 4) and intracellular signaling domain. In some embodiments, the CAR binds to a target antigen and induces cytotoxicity upon antigen binding. In some embodiments, the immunomodulator is an agent that is capable of regulating immune function of the NK cell. In some embodiments, an immunomodulator may be an immunoactivator. In other embodiments, an immunomodulator may be an immunosuppressant. In some embodiments, the immunomodulator is an exogenous cytokine, such as an interleukin or a functional portion thereof. Exemplary features of a CAR and immunomodulators are further described in the following subsections.

In some embodiments, the g-NK cells may be further engineered by gene editing as described in Section III.

A. Antigen Receptor, e.g. Chimeric Antigen Receptor

In provided embodiments, the g-NK cells are genetically engineered to express an antigen receptor(s) that binds to an antigen of interest. In certain embodiments, the antigen receptor is a chimeric antigen receptor (CAR). In certain embodiments, the antigen receptor is a T-cell receptor (TCR). The antigen receptor can bind to, for example, a tumor specific or tumor associated antigen or a pathogen antigen. Thus, the engineered antigen receptor, e.g. CAR, is a recombinant antigen receptor that is intended to introduce a certain antigen specificity to the NK cell. In some embodiments, the antigen receptor, such as a CAR, is stably integrated into the g-NK cell. In other embodiments, the antigen receptor, e.g. CAR is transiently expressed by the g-NK cell. For instance, the g-NK cells comprise a CAR with a defined polypeptide sequence expressed from an exogenous polynucleotide that has been introduced into the immune effector cell, either transiently or integrated into the genome. In provided embodiments, the engineered NK cells provided herein that comprise an antigen receptor (e.g. CAR) may be used for immunotherapy to target and destroy cells associated with a disease or disorder, e.g. cancer cells, that express the target antigen recognized by the antigen receptor (e.g. CAR).

In some embodiments, the antigen receptor is a chimeric antigen receptor (CAR). The CAR is typically encoded by a nucleic acid sequence (polynucleotide) that comprises a leader sequence, an extracellular targeting domain (also called ectodomain; e.g. antigen binding domain, such as an scFv), a transmembrane domain and one or more intracellular signaling domains. In some embodiments, a CAR is a fusion protein that includes an extracellular targeting domain (ectodomain) comprising an antigen recognition or antigen binding domain; a transmembrane domain; and an intracellular signaling domain. The ectodomain and transmembrane domains may be linked by a flexible linker (also called a spacer). In some embodiments, the antigen binding domain, such as a single-chain variable fragment (scFv) derived from a monoclonal antibody, recognizes a target antigen. In some embodiments, the antigen binding domain, e.g. an scFv, is linked or fused to the transmembrane domain via a spacer. In some embodiments, the intracellular signaling domain includes an immunoreceptor tyrosine-based activation motif (ITAM). Activation of the CAR fusion protein results in cellular activation in response to recognition by the scFv (or other antigen binding domain) of its target. When a cell expresses such a CAR, it can recognize and kill target cells that express the target antigen. This property makes CAR-expressing cells particularly attractive agents for specific targeting of cellular activity to aberrant cells, including, but not limited to, cancer cells. Various CARs have been developed against target antigens, including tumor associated antigens, for expression in various immune cells, including T lymphocytes and Natural Killer (NK) cells, to mediate cytotoxic activity against target cells expressing the antigen and can be the engineered g-NK cells disclosed herein.

In some embodiments, the leader sequence can be any of the signal peptide sequences described herein. An exemplary CD8α signal peptide is set forth in SEQ ID NO:12. An exemplary GM-CSFRa signal peptide is set forth in SEQ ID NO:13. An exemplary IgK signal peptide is set forth in SEQ ID NO:14. An exemplary IgK signal peptide is set forth in SEQ ID NO: 43.

Any variety of chimeric antigen receptor can be expressed in the engineered NK cells, including those described in International PCT Application PCT/US2018/024650, PCT/IB2019/000141, PCT/IB2019/000181, and/or PCT/US2020/020824, PCT/US2020, 035752.

In certain embodiments, the extracellular antigen-binding domain specifically binds to an antigen. In some embodiments, the extracellular antigen-binding domain or targeting domain is derived from an antibody molecule, and comprises one or more complementarity determining regions (CDRs) from an antibody molecule that confer antigen specificity on the CAR. In certain embodiments, the extracellular antigen-binding domain is a single chain variable fragment (scFv). In certain embodiments, the scFv is a human scFv. In certain embodiments, the scFv is a humanized scFv. In certain embodiments, the extracellular antigen-binding domain is a Fab, which is optionally crosslinked. In certain embodiments, the extracellular binding domain is a F(ab′)₂. In certain embodiments, any of the foregoing molecules may be comprised in a fusion protein with a heterologous sequence to form the extracellular antigen-binding domain. In certain embodiments, the scFv is identified by screening scFv phage library with an antigen-Fc fusion protein.

In some embodiments, the scFv comprises the variable chain portion of an immunoglobulin light chain and an immunoglobulin heavy chain molecule separated by a flexible linker polypeptide. The order of the heavy and light chains is not limiting and can be reversed. The flexible polypeptide linker allows the heavy and light chains to associate with one another and reconstitute an immunoglobulin antigen binding domain. In some embodiments, the flexible linker is a GS linker, such as set forth in SEQ ID NO: 56. In some embodiments, the flexible linker is a Whitlow linker, such as set forth in SEQ ID NO: 55. Suitably, the light chain variable region comprises three CDRs and the heavy chain variable region comprises three CDRs. Suitably, the CDRs for use in the antigen-binding targeting domain are derived from an antibody molecule of any species (e.g., human, mouse, rat, rabbit, goat, sheep) and the framework regions between the CDRs are humanized or comprise a sequence that is at least 85%, 90%, 95 or 99% identical to a human framework region.

When the targeting domain of the CAR comprises an scFv, the immunoglobulin light chain and the immunoglobulin heavy chain are joined by polypeptide linkers of various lengths. Suitably, the polypeptide linker comprises a length greater than or equal to 10 amino acids. Suitably, the polypeptide linker comprises a length greater than 10, 15, 20, or 25 amino acids. Suitably, the polypeptide linker comprises a length less than or equal to 30 amino acids. Suitably, the polypeptide linker comprises a length less than 15, 20, 25, or 30 amino acids. Suitably, the polypeptide linker comprises between 10 and 30 amino acids in length. Suitably, the polypeptide linker comprises between 10 and 25 amino acids in length. Suitably, the polypeptide linker comprises between 10 and 20 amino acids in length. Suitably, the polypeptide linker comprises between 10 and 15 amino acids in length. Suitably, the polypeptide linker comprises between 15 and 30 amino acids in length. Suitably, the polypeptide linker comprises between 20 and 30 amino acids in length. Suitably, the polypeptide linker comprises between 25 and 30 amino acids in length. Suitably, the polypeptide linker comprises hydrophilic amino acids. Suitably, the polypeptide linker consists of hydrophilic amino acids. Suitably, the polypeptide linker comprises a G₄S sequence (GGGGS). The G₄S linker allows for flexibility and protease resistance of the linker. Suitably, the G₄S linker is consecutively repeated in the polypeptide linker 1, 2, 3, 4, 5, 6, 7, or 8 times.

In certain embodiments, the antigen is a tumor antigen. In certain embodiments, the antigen is a pathogen antigen, including for example, a viral or a bacterial antigen.

Binding of an extracellular antigen-binding domain (for example, an scFv or an analog thereof) of an antigen-targeted CAR can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March 1986, which is incorporated by reference herein). The radioactive isotope can be detected by such means as the use of a gamma counter or a scintillation counter or by autoradiography. In certain embodiments, the extracellular antigen-binding domain of the CAR is labeled with a fluorescent marker. Non-limiting examples of fluorescent markers include green fluorescent protein (GFP), blue fluorescent protein (e.g., EBFP, EBFP2, Azurite, and mKalamal), cyan fluorescent protein (e.g., ECFP, Cerulean, and CyPet), and yellow fluorescent protein (e.g., YFP, Citrine, Venus, and YPet).

In certain embodiments, the antigen recognizing receptor binds to a tumor associated or tumor specific antigen. Any suitable tumor associated or tumor specific antigen (e.g., an antigenic peptide) can be used in the embodiments described herein. The antigen can be, but is not limited to, a protein, non-protein, neoantigen, post-translationally modified antigen, peptide-MHC antigen, and/or over-expressed antigen.

For example, tumor targets include, but are not limited to CD38 (multiple myeloma); CD20 (lymphoma); epidermal growth factor receptor (EGFR; non-small cell lung cancer, epithelial carcinoma, and glioma); variant III of the epidermal growth factor receptor (EGFRvIII; glioblastoma); human epidermal growth factor receptor 2 (HER2; ovarian cancer, breast cancer, glioblastoma, colon cancer, osteosarcoma, and medulloblastoma); mesothelin (mesothelioma, ovarian cancer, and pancreatic adenocarcinoma); prostate-specific membrane antigen (PSMA; prostate cancer); carcinoembryonic antigen (CEA; pancreatic adenocarcinoma, breast cancer, and colorectal carcinoma); disialoganglioside 2 (GD2; neuroblastoma and melanoma); interleukin-13Ra2 (glioma); glypican-3 (hepatocellular carcinoma); carbonic anhydrase IX (CAIX; renal cell carcinoma); L 1 cell adhesion molecule (L1-CAM; neuroblastoma, melanoma, and ovarian adenocarcinoma); cancer antigen 125 (CA 125; epithelial ovarian cancer); CD133 (glioblastoma and cholangiocarcinoma); fibroblast activation protein (FAP; malignant pleural mesothelioma); cancer/testis antigen 1B (CTAG1B; melanoma and ovarian cancer); mucin 1 (seminal vesicle cancer); and folate receptor-a (FR-a; ovarian cancer).

Further non-limiting examples of a tumor antigen include, but are not limited to, Non-limiting examples of tumor antigens include carbonic anhydrase IX (CAIX), carcinoembryonic antigen (CEA), CD8, CD7, CD10, CD19, CD20, CD22, CD30, CD33, CLL1, CD34, CD38, CD41, CD44, CD49c, CD49f, CD56, CD66c, CD73, CD74, CD104, CD133, CD138, CD123, CD142, CD44V6, an antigen of a cytomegalovirus (CMV) infected cell (e.g., a cell surface antigen), cutaneous lymphocyte-associated antigen (CLA; a specialized glycoform of P-selectin glycoprotein ligand-1 (PSGL-1)), epithelial glycoprotein-2 (EGP-2), epithelial glycoprotein-40 (EGP-40), epithelial cell adhesion molecule (EpCAM), receptor tyrosine-protein kinases erb-B2,3,4 (erb-B2,3,4), folate-binding protein (EBP), fetal acetylcholine receptor (AChR), folate receptor-alpha, Ganglioside G2 (GD2), Ganglioside G3 (GD3), human Epidermal Growth Factor Receptor 2 (HER2), human telomerase reverse transcriptase (hTERT), Interleukin-13 receptor subunit alpha-2 (IL-13Ralpha2), kappa-light chain, kinase insert domain receptor (KDR), Lewis Y (LeY), LI cell adhesion molecule (L1CAM), melanoma antigen family A, 1 (MAGE-A1), Mucin 16 (MUC16), Mucin 1 (MUC1), Mesothelin (MSLN), ERBB2, MAGE A3, p53, MARTI, GP100, Proteinase3 (PR1), Tyrosinase, Survivin, hTERT, EphA2, an NKG2D ligand, cancer-testis antigen NY-ESO-1, oncofetal antigen (h5T4), prostate stem cell antigen (PSCA), prostate-specific membrane antigen (PSMA), ROR1, tetraspanin 8 (TSPAN8), tumor-associated glycoprotein 72 (TAG-72), vascular endothelial growth factor R2 (VEGF-R2), Wilms tumor protein (WT-1), cytokine receptor-like factor 2 (CRLF2), BCMA, GPC3, NKCS1, EGF1R, EGFR-VIII, and ERBB.

In some embodiments, the tumor antigen is CD19, ROR1, Her2, PSMA, PSCA, mesothelin (MSLN), or CD20. In some embodiments, the tumor antigen is CD19, CD20, CD33, MSLN, or cytokine receptor-like factor 2 (CRLF2), which are expressed on leukemias or lymphomas. In some embodiments, the CAR binds a target antigen selection from Her2, EGFR, alpha folate receptor, CEA, cMET, MUC2, Mesothelin, or ROR1. In a certain embodiment, the target antigen is CD38, CD319/SLAMF-7, TNFRSF 17/BCMA, SYND1/CD138, CD229, CD47, Her2/Neu, epidermal growth factor receptor (EGFR), CD123/IL3-RA, CD19, CD20, CD22, Mesothelin, EpCAM, MUC1, MUC 16, Tn antigen, NEU5GC, NeuGcGM3, GD2, CLL-1, or HERV-K. In some embodiments, the target antigen is a blood cancer associated antigen. For instance, the target antigen may be CD38, CD319/SLAMF-7, TNFRSF 17/BCMA, SYND1/CD138, CD229, CD47, CD123/IL3-RA, CD19, CD20, CD22, or CLL-1.

A variety of antigen-binding domains for incorporation into a CAR are known. In one non-limiting example, the g-NK cell is engineered with a CD38 specific CAR (see e.g. WO2018/104562).

In some embodiments, the g-NK cell is engineered with a bispecific CAR or multiple different CARs, wherein their affinity is for two distinct ligands/antigens. Bispecific CAR-NKs can be used either for increasing the number of potential binding sites on cancer cells or, alternatively, for localizing cancer cells to other immune effector cells which express ligands specific to the NK-CAR. For use in cancer therapy, a bispecific CAR may bind to a target tumor cell and to an effector cell, e.g. a T cell, NK cell or macrophage. Thus, for example, in the case of multiple myeloma, a bispecific CAR may bind a T cell antigen (e.g. CD3, etc.) and a tumor cell marker (e.g. CD38, etc.). A bispecific CAR may alternatively bind to two separate tumor cell markers, increasing the overall binding affinity of the NK cell for the target tumor cell. This may reduce the risk of cancer cells developing resistance by downregulating one of the target antigens. An example in this case, in multiple myeloma, would be a CAR binding to both CD38 and CS-1/SLAMF7. Another tumor cell marker suitably targeted by the CAR is a “don't eat me” type marker on tumors, exemplified by CD47.

In some embodiments, the engineered g-NK cells may comprise a bispecific CAR or multiple CARs expressed by the same NK cell. This allows the NK cells to target two different antigens simultaneously. Suitably, the bispecific CAR has specificity for any two of the following antigens: CD38, CD319/SLAMF-7, TNFRSF 17/BCMA, CD123/IL3-RA, SYND1/CD138, CD229, CD47, Her2/Neu, epidermal growth factor receptor (EGFR), CD19, CD20, CD22, Mesothelin, EpCAM, MUC1, MUC16, Tn antigen, NEU5GC, NeuGcGM3, GD2, CLL-1, CD 123, HERV-K. Suitably, the bispecific nature of the CAR NK cell may allow binding to a tumor antigen and another immune cell, such as a T cell or dendritic cell. Suitably, the bispecific nature of the CAR NK cell may allow binding to a checkpoint inhibitor, such as PDL-1, or CD47. Suitably, the first CAR has CD38 specificity, and the second CAR has specificity for any one of SLAMF-7, BCMA, CD138, CD229, PDL-1, or CD47. Suitably, the first CAR has specificity for CD38, and the second CAR has specificity for SLAMF-7, BCMA, CD138, CD229. Suitably, the first CAR has specificity for CD38, and the second CAR has specificity for SLAMF-7. Suitably, the first CAR has specificity for CD38, and the second CAR has specificity for BCMA. Suitably, the first CAR has specificity for CD38, and the second CAR has specificity for CD 138. Suitably, the first CAR has specificity for CD38, and the second CAR has specificity for CD229.

In some embodiments, the transmembrane domain of the CAR comprises hydrophobic amino acid residues and allows the CAR to be anchored into the cell membrane of the engineered NK cell. Suitably, the transmembrane domain comprises an amino acid sequence derived from a transmembrane protein. Suitably, the transmembrane domain comprises an amino acid sequence derived from the transmembrane domain of the alpha, beta, or zeta chain of the T-cell receptor, CD27, CD28, CD3 epsilon, CD45, CD4, CD5, CD8, CD9, CD16, CD22, CD33, CD37, CD64, CD80, CD86, CD 134, CD 137, and CD 154. Suitably, the CAR comprises a transmembrane with an amino acid sequence derived from the transmembrane domain of CD8. Suitably, the CAR comprises a transmembrane domain with an amino acid sequence derived from the transmembrane domain of human CD8 alpha. In some embodiments, the CAR contains a transmembrane domain of CD8 alpha that has the sequence of amino acids set forth in SEQ ID NO:61 or a sequence of amino acids that exhibits at least 85%, 90% or 95% sequence identity to SEQ ID NO:61. In some embodiments, the transmembrane domain is set forth in SEQ ID NO:61. In some embodiments, suitably, the CAR comprises a transmembrane with an amino acid sequence derived from the transmembrane domain of CD28. Suitably, the CAR comprises a transmembrane domain with an amino acid sequence derived from the transmembrane domain of human CD28. In some embodiments, the CAR contains a transmembrane domain of CD28 that has the sequence of amino acids set forth in SEQ ID NO:39 or a sequence of amino acids that exhibits at least 85%, 90% or 95% sequence identity to SEQ ID NO:39. In some embodiments, the transmembrane domain is set forth in SEQ ID NO:39.

In some embodiments, the CARs can also comprise a spacer region located between the antigen-binding targeting domain and the transmembrane domain. In some embodiments, the spacer region comprises hydrophilic amino acids and allows flexibility of the targeting domain with respect to the cell surface. Suitably, the spacer region comprises greater than 5, 10, 15, 20, 25, or 30 amino acids. Suitably, the spacer region comprises less than 10, 15, 20, 25, 30, or 35 amino acids. In some embodiments, the spacer region is a hinge region and includes a hinge sequence of CD8 or of an immunoglobulin molecule.

In some embodiments, the spacer region is or includes the CD8 hinge. In some embodiments the spacer is the hinge region of human CD8. In some embodiments, the CAR contains a CD8 hinge spacer sequence that has the sequence of amino acids set forth in SEQ ID NO:60 or a sequence of amino acids that exhibits at least 85%, 90% or 95% sequence identity to SEQ ID NO:60. In some embodiments, the sequence of the spacer is set forth in SEQ ID NO:60.

In some embodiments, the spacer region includes all or a portion containing the hinge domain of an IgG1 Fc or an IgG4 Fc. In some embodiments, the spacer is an IgG4 Fc spacer. In some embodiments, the CAR contains an IgG4 Fc spacer that has the sequence of amino acids set forth in SEQ ID NO:38 or a sequence of amino acids that exhibits at least 85%, 90% or 95% sequence identity to SEQ ID NO:38. In some embodiments, the sequence of the spacer is set forth in SEQ ID NO:38. In some embodiments, the sequence of the spacer is the hinge portion of the IgG1 Fc or IgG4 Fc. In some embodiments, the CAR contains an IgG4 hinge spacer. In some embodiments, the IgG4 hinge spacer has the sequence of amino acids set forth in SEQ ID NO: 59 or a sequence of amino acids that exhibits at least 85%, 90% or 95% sequence identity to SEQ ID NO:59. In some embodiments, the sequence of the spacer is set forth in SEQ ID NO:59.

In some embodiments, the intracellular signaling domain of the CAR increases the potency of the CAR and comprises an intracellular signaling domain derived from a protein involved in immune cell signal transduction. Suitably, the one or more intracellular signaling domains comprise an intracellular signaling domain derived from CD3 zeta CD28, OX-40, 4-1BB, DAP10, DAP 12, 2B4 (CD244), or any combination thereof. Suitably, the one or more intracellular signaling domains comprise an intracellular signaling domain derived from any two of CD3 zeta CD28, OX-40, 4-1BB, DAP10, DAP 12, 2B4 (CD244), or any combination thereof.

In some embodiments, the endodomain of a CAR may include two more signaling domains. For instance, a CAR may include a primary intracellular signaling domain, such as a CD3zeta intracellular signaling domain, and an intracellular signaling domains from a costimulatory molecule to provide additional signal to the cells, such as to further enhance potency of the CAR-expressing immune cell. Thus, in some embodiments, the chimeric antigen receptor (CAR) comprises s: 1) an antigen binding domain; 2) a flexible linker; 3) a transmembrane region; and 4) an intracellular signaling region comprising a first primary intracellular signaling domain, such as a CD3 zeta intracellular signaling domain and second co-stimulatory intracellular signaling domain. In some embodiments, a costimulatory domain can be CD27, CD28, 4-1BB (CD137), 0X40 (CD134), CD30, CD40, lymphocyte function-associated antigen-1 (LFA-1), CD2, CD7, LIGHT, NKG2C, and/or B7-H3 costimulatory domains. In some embodiments, a costimulatory domain can be CD27, CD28, 4-1BB (CD137), 0X40 (CD134), DAP10, DAP12, ICOS, and/or 2B4. In some embodiments, a co-stimulatory domain can be CD27, CD28, 4-1BB, 2B4, DAP10, DAP12, 0X40, CD30, CD40, lymphocyte function-associated antigen-1 (LFA-1), CD2, CD7, LIGHT, NKG2C, and/or B7-H3 costimulatory domains. In some embodiments, the costimulatory signaling domain is a signaling domain of CD28. In some embodiments, the costimulatory signaling domain is a signaling domain of 4-1BB.

In some embodiments, the CAR contains an intracellular signaling domain that contains a signaling domain of CD3zeta that has the sequence of amino acids set forth in SEQ ID NO:41 or a sequence of amino acids that exhibits at least 85%, 90% or 95% sequence identity to SEQ ID NO:41. In some embodiments, the CAR contains an intracellular signaling domain that contains the signaling domain of CD3zeta that has the sequence of amino acids set forth in SEQ ID NO:41. In some embodiments, the CAR contains an intracellular signaling domain that contains a signaling domain of CD3zeta that has the sequence of amino acids set forth in SEQ ID NO:50 or a sequence of amino acids that exhibits at least 85%, 90% or 95% sequence identity to SEQ ID NO:50. In some embodiments, the CAR contains an intracellular signaling domain that contains a signaling domain of CD3zeta that has the sequence of amino acids set forth in SEQ ID NO:50.

In some embodiments, the CAR contains an intracellular signaling domain that contains a costimulatory signaling domain of CD28 that has the sequence of amino acids set forth in SEQ ID NO:40 or a sequence of amino acids that exhibits at least 85%, 90% or 95% sequence identity to SEQ ID NO:40. In some embodiments, the CAR contains an intracellular signaling domain that contains a costimulatory signaling domain of CD28 that has the sequence of amino acids set forth in SEQ ID NO:40. In some embodiments, the CAR contains an intracellular signaling domain that contains a costimulatory signaling domain of CD28 that has the sequence of amino acids set forth in SEQ ID NO:52 or a sequence of amino acids that exhibits at least 85%, 90% or 95% sequence identity to SEQ ID NO:52. In some embodiments, the CAR contains an intracellular signaling domain that contains the costimulatory signaling domain of CD28 that has the sequence of amino acids set forth in SEQ ID NO:52.

In some embodiments, the CAR contains an intracellular signaling domain that contains a costimulatory signaling domain of 4-1BB that has the sequence of amino acids set forth in SEQ ID NO:51 or a sequence of amino acids that exhibits at least 85%, 90% or 95% sequence identity to SEQ ID NO:51. In some embodiments, the CAR contains an intracellular signaling domain that contains a costimulatory signaling domain of 4-1BB that has the sequence of amino acids set forth in SEQ ID NO:51.

In some embodiments, an intracellular signaling domain can be a domain of CD3zeta, CD28 and/or 4-1BB.

Suitably, the CAR comprises at least two intracellular signaling domains derived from CD3 zeta and 4-1BB. In some embodiments, the CAR comprises an intracellular signaling domain comprising the sequence set forth in SEQ ID NO: 41 and SEQ ID NO:51. In some embodiments, the CAR comprises an intracellular signaling domain comprising the sequence set forth in SEQ ID NO: 50 and SEQ ID NO:51.

In other embodiments, suitably the CAR comprises at least two intracellular signaling domains derived from CD3 zeta and CD28. In some embodiments, the CAR comprises an intracellular signaling domain comprising the sequence set forth in SEQ ID NO: 41 and SEQ ID NO:40. In some embodiments, the CAR comprises an intracellular signaling domain comprising the sequence set forth in SEQ ID NO: 41 and SEQ ID NO:52. In some embodiments, the CAR comprises an intracellular signaling domain comprising the sequence set forth in SEQ ID NO: 50 and SEQ ID NO:40. In some embodiments, the CAR comprises an intracellular signaling domain comprising the sequence set forth in SEQ ID NO: 50 and SEQ ID NO:52.

In some embodiments, the antigen receptor (e.g. CAR) is encoded by a polynucleotide that encodes a CAR with an NH₂-terminal leader sequence. The leader sequence (also known as the signal peptide) allows the expressed CAR construct to enter the endoplasmic reticulum (ER) and target the cell surface. The leader sequence is cleaved in the ER and the mature cell surface CAR does not possess a leader sequence. In general, the leader sequence length will be in the range of 5 to 30 amino acids, and comprise a stretch of hydrophobic amino acids. Suitably, the leader sequence comprises greater than 5, 10, 15, 20, or 25 amino acids in length. Suitably, the leader sequence comprises less than 10, 15, 20, 25, or 30 amino acids in length. Suitably, the leader sequence comprises a sequence derived from any secretory protein. Suitably, the leader sequence comprises a sequence derived from the CD8 alpha leader sequence. In some embodiments, suitably the leader sequence comprises a sequence derived from the IgK leader sequence. In some embodiments, the leader sequence is set forth in SEQ ID NO:43.

In some embodiments, the CAR is the CAR present in any of a variety of known engineered cell products. The CAR may include, but is not limited to a CAR engineered into cells of ABECMA®, JCARH125, CARVYKTI™ (NJ-68284528; Janssen/Legend), P-BCMA-101 (Poseida), PBCAR269A (Poseida), P-BCMA-Allo1 (Poseida), Allo-715 (Pfizer/Allogene), CT053 (Carsgen), Descartes-08 (Cartesian), PHE885 (Novartis), CTX120 (CRISPR Therapeutics); YESCARTA®, KYMRIAH®, TECARTUS®, or BREYANZI®.

In some embodiments, the CAR comprises a CAR of a commercial CAR cell therapy. Non-limiting examples of a CAR in commercial cell based therapies include the CAR engineered in cells of brexucabtagene autoleucel (TECARTUS®), axicabtagene ciloleucel (YESCARTA®), idecabtagene vicleucel (ABECMA®), ciltacabtagene autoleucel (CARVYKTI™), lisocabtagene maraleucel (BREYANZI®), tisagenlecleucel (KYMRIAH®).

In some embodiments, the g-NK cell is engineered with a CAR that binds to CD19. Cluster of Differentiation 19 (CD 19) is an antigenic determinant detectable on leukemia precursor cells. The human and murine amino acid and nucleic acid sequences can be found in a public database, such as GenBank, UniProt and Swiss-Prot. For example, the amino acid sequence of human CD19 can be found as UniProt/Swiss-Prot Accession No. P15391 and the nucleotide sequence encoding of the human CD19 can be found at Accession No. NM_001178098. CD19 is expressed on most B lineage cancers, including, e.g., acute lymphoblastic leukemia, chronic lymphocyte leukemia and non-Hodgkin's lymphoma. It is also an early marker of B cell progenitors. See, e.g., Nicholson et al. Mol. Immun. 34 (16-17): 1157-1165 (1997). The antigen-binding extracellular domain in the CAR polypeptide disclosed herein is specific to CD19 (e.g., human CD19). In some examples, the antigen-binding extracellular domain may comprise a scFv extracellular domain capable of binding to CD 19. In some embodiments, an anti-CD19 CAR may comprise an anti-CD19 single-chain variable fragment (scFv) specific for CD19, followed by a spacer and transmembrane domain that is fused to an intracellular co-signaling domain (e.g., a CD28 or 4-1BB) and a CD3zeta signaling domain.

In some embodiments, the extracellular binding domain of the CD19 CAR may comprise the heavy chain variable region (V_{H}) set forth in SEQ ID NO:54 and the light chain variable region (V_{L}) set forth in SEQ ID NO:53. In some embodiments, the linker separating the VH and VL in the scFv is a GS linker, such as set forth in SEQ ID NO: 56. In some embodiments, the linker separating the VH and VL in the scFv is the Whitlow linker set forth in SEQ ID NO:55. In some embodiments, the scFv has the sequence of amino acids set forth in SEQ ID NO:57. In some embodiments, the scFv has the sequence of amino acids set forth in SEQ ID NO:58. In some embodiments, the spacer is a CD8 hinge, such as set forth in SEQ ID NO: 60. In some embodiments, the spacer is an IgG4 hinge, such as set forth in SEQ ID NO: 59. In some embodiments, the intracellular signaling domain contains a 4-1BB costimulatory signaling domain and a CD3 zeta signaling domain, such as any described herein. In some embodiments, the intracellular signaling domain contains a CD28 costimulatory signaling domain and a CD3 zeta signaling domain, such as any described herein. In some embodiments, it is understood the CAR includes any sequences that exhibit some sequence variation to any of the above or described SEQ ID NOS, such as at least 85%, 90%, 95% or more sequence identity thereto, and retain binding to CD19 and intracellular signaling and cytotoxic activity.

In some embodiments, the CAR comprises an anti-CD19 CAR of a commercial CAR cell therapy. Non-limiting examples of an anti-CD19 CAR in commercial cell based therapies include the anti-CD19 CAR engineered in cells of YESCARTA®, KYMRIAH®, TECARTUS®, or BREYANZI®.

CD20 is a proven therapeutic target for hematotolic malignancies, such as B-NHL, supported by approved and widely used monoclonal antibody therapy. Further, the universal presence of CD19, CD20, and CD22 antigens on malignant B-cells make them the perfect targets for cellular therapies. In some embodiments, the CAR contains an extracellular antigen-binding domain that binds to CD20. In a particular embodiment, the CD20 CAR comprise a CAR directed to CD20, wherein the CAR directed to CD20 comprises a single chain Fv antibody or antibody fragment (scFv). In some embodiments, an anti-CD20 CAR may comprise an anti-CD20 single-chain variable fragment (scFv) specific for CD20, followed by a spacer and transmembrane domain that is fused to an intracellular co-signaling domain (e.g., a CD28 or 4-1BB) and a CD3zeta signaling domain. In some embodiments, the CAR contains an anti-CD20 scFv, followed by a IgG4-Fc spacer, a CD28 transmembrane domain, a 4-1BB costimulatory domain and a CD3 zeta signaling domain. In some embodiments, the CAR is the Leu16 CAR as described in Rufener et al. Cancer Immunol. Res. 2016 4:509-519. See also, GenBank accession #KX055828).

In some embodiments, the extracellular binding domain of the CD20 CAR may comprise the heavy chain variable region (V_{H}) set forth in SEQ ID NO:36 and the light chain variable region (V_{L}) set forth in SEQ ID NO:35. In some embodiments, the linker separating the VH and VL in the scFv is a GS linker, such as set forth in SEQ ID NO: 56. In some embodiments, the linker separating the VH and VL in the scFv is the Whitlow linker set forth in SEQ ID NO:55. In some embodiments, the anti-CD20 scFv is set forth in SEQ ID NO: 37. In some embodiments, the intracellular signaling domain contains a 4-1BB costimulatory signaling domain and a CD3 zeta signaling domain, such as any described herein. In some embodiments, the intracellular signaling domain contains a CD28 costimulatory signaling domain and a CD3 zeta signaling domain, such as any described herein. In some embodiments, it is understood the CAR includes any sequences that exhibit some sequence variation to any of the above or described SEQ ID NOS, such as at least 85%, 90%, 95% or more sequence identity thereto, and retain binding to CD38 and intracellular signaling and cytotoxic activity. In some embodiments, the anti-CD20 CAR contains the scFv set forth in SEQ ID NO: 37, and IgG4 Fc spacer (e.g. SEQ ID NO: 38), a CD28 transmembrane domain (e.g. SEQ ID NO: 39), a CD28 costimulatory signaling domain (e.g. SEQ ID NO: 40), and a CD3 zeta signaling domain (e.g. SEQ ID NO:41). In some embodiments, the CD20 CAR has the sequence of amino acids set forth in SEQ ID NO:42 or a sequence that exhibits at least 85%, at least 90% or at least 95% sequence identity to SEQ ID NO: 42. In some embodiments, the CD20 CAR has the sequence set forth in SEQ ID NO: 42. In some embodiments, the CAR is encoded by a polynucleotide (e.g. mRNA) set forth in SEQ ID NO:45.

In some embodiments, the g-NK cell is engineered with a CAR that binds to BCMA. BCMA RNA has been detected universally in multiple myeloma cells and in other lymphomas, and BCMA protein has been detected on the surface of plasma cells from multiple myeloma patients by several investigators (see, e.g., Novak et al., Blood, 103(2): 689-694, 2004; Neri et al., Clinical Cancer Research, 73(19): 5903-5909, 2007; Bellucci et al., Blood, 105(10): 3945-3950, 2005; and Moreaux et al., Blood, 703(8): 3148-3157, 2004. CARs for targeting BCMA are known and include, but are not limited to, those described in U.S. Pat. No. 10,934,363 or WO 2018/028647. In some embodiments, the CAR contains an extracellular antigen-binding domain that binds to BCMA. In a particular embodiment, the BCMA CAR comprise a CAR directed to BCMA, wherein the CAR directed to BCMA comprises a single chain Fv antibody or antibody fragment (scFv). In some embodiments, an anti-BCMA CAR may comprise an anti-BCMA single-chain variable fragment (scFv) specific for BCMA, followed by a spacer and transmembrane domain that is fused to an intracellular co-signaling domain (e.g., a CD28 or 4-1BB) and a CD3zeta signaling domain.

In some embodiments, the extracellular binding domain of the BCMA CAR comprises an scFv derived from C11D5.3, a murine monoclonal antibody as described in Carpenter et al., Clin. Cancer Res. 19(8):2048-2060 (2013). See also PCT Application Publication No. WO2010/104949. The C11D5.3-derived scFv may comprise the heavy chain variable region (V_{H}) and the light chain variable region (V_{L}) of C11D5.3. In some embodiments, the VH has the sequence of amino acids set forth in SEQ ID NO: 63 and the VL has the sequence of amino acids set forth in SEQ ID NO: 62. In some embodiments, the linker separating the VH and VL in the scFv is a GS linker, such as set forth in SEQ ID NO: 56. In some embodiments, the linker separating the VH and VL in the scFv is the Whitlow linker set forth in SEQ ID NO:55. In some embodiments, the scFv has the sequence of amino acids set forth in SEQ ID NO:64. In some embodiments, the intracellular signaling domain contains a 4-1BB costimulatory signaling domain and a CD3 zeta signaling domain, such as any described herein. In some embodiments, the intracellular signaling domain contains a CD28 costimulatory signaling domain and a CD3 zeta signaling domain, such as any described herein. In some embodiments, it is understood the CAR includes any sequences that exhibit some sequence variation to any of the above or described SEQ ID NOS, such as at least 85%, 90%, 95% or more sequence identity thereto, and retain binding to BCMA and intracellular signaling and cytotoxic activity.

In some embodiments, the extracellular binding domain of the BCMA CAR comprises an scFv derived from another murine monoclonal antibody, C12A3.2, as described in Carpenter et al., Clin. Cancer Res. 19(8):2048-2060 (2013) and PCT Application Publication No. WO2010/104949. In some embodiments, the VH has the sequence of amino acids set forth in SEQ ID NO: 66 and the VL has the sequence of amino acids set forth in SEQ ID NO: 65. In some embodiments, the linker separating the VH and VL in the scFv is a GS linker, such as set forth in SEQ ID NO: 56. In some embodiments, the linker separating the VH and VL in the scFv is the Whitlow linker set forth in SEQ ID NO:55. In some embodiments, the scFv has the sequence of amino acids set forth in SEQ ID NO:67. In some embodiments, the intracellular signaling domain contains a 4-1BB costimulatory signaling domain and a CD3 zeta signaling domain, such as any described herein. In some embodiments, the intracellular signaling domain contains a CD28 costimulatory signaling domain and a CD3 zeta signaling domain, such as any described herein. In some embodiments, it is understood the CAR includes any sequences that exhibit some sequence variation to any of the above or described SEQ ID NOS, such as at least 85%, 90%, 95% or more sequence identity thereto, and retain binding to BCMA and intracellular signaling and cytotoxic activity.

In some embodiments, the extracellular binding domain of the BCMA CAR comprises a murine monoclonal antibody with high specificity to human BCMA, referred to as BB2121 in Friedman et al., Hum. Gene Ther. 29(5):585-601 (2018)). See also, PCT Application Publication No. WO2012163805. BB2121 is also known as anti-BCMA02 CAR. In some embodiments, the VH has the sequence of amino acids set forth in SEQ ID NO: 68 and the VL has the sequence of amino acids set forth in SEQ ID NO: 69. In some embodiments, the linker separating the VH and VL in the scFv is a GS linker, such as set forth in SEQ ID NO: 56. In some embodiments, the linker separating the VH and VL in the scFv is the Whitlow linker set forth in SEQ ID NO:55. In some embodiments, the scFv has the sequence of amino acids set forth in SEQ ID NO:70. In some embodiments, the intracellular signaling domain contains a 4-1BB costimulatory signaling domain and a CD3 zeta signaling domain, such as any described herein. In some embodiments, the intracellular signaling domain contains a CD28 costimulatory signaling domain and a CD3 zeta signaling domain, such as any described herein. In some embodiments, it is understood the CAR includes any sequences that exhibit some sequence variation to any of the above or described SEQ ID NOS, such as at least 85%, 90%, 95% or more sequence identity thereto, and retain binding to BCMA and intracellular signaling and cytotoxic activity.

In some embodiments, the extracellular binding domain of the BCMA CAR comprises single variable fragments of two heavy chains (VHH) that can bind to two epitopes of BCMA as described in Zhao et al., J. Hematol. Oncol. 11(1):141 (2018), also referred to as LCAR-B38M. See also, PCT Application Publication No. WO2018/028647. In some embodiments, the intracellular signaling domain contains a 4-1BB costimulatory signaling domain and a CD3 zeta signaling domain, such as any described herein. In some embodiments, the intracellular signaling domain contains a CD28 costimulatory signaling domain and a CD3 zeta signaling domain, such as any described herein. In some embodiments, it is understood the CAR includes any sequences that exhibit some sequence variation to any of the above or described SEQ ID NOS, such as at least 85%, 90%, 95% or more sequence identity thereto, and retain binding to BCMA and intracellular signaling and cytotoxic activity.

In some embodiments, the extracellular binding domain of the BCMA CAR comprises a fully human heavy-chain variable domain (FHVH) as described in Lam et al., Nat. Commun. 11(1):283 (2020), also referred to as FHVH33. In some embodiments, the intracellular signaling domain contains a 4-1BB costimulatory signaling domain and a CD3 zeta signaling domain, such as any described herein. In some embodiments, the intracellular signaling domain contains a CD28 costimulatory signaling domain and a CD3 zeta signaling domain, such as any described herein. In some embodiments, it is understood the CAR includes any sequences that exhibit some sequence variation to any of the above or described SEQ ID NOS, such as at least 85%, 90%, 95% or more sequence identity thereto, and retain binding to BCMA and intracellular signaling and cytotoxic activity.

In some embodiments, the CAR comprises an anti-BCMA CAR of a commercial CAR cell therapy. Non-limiting examples of an anti-BCMA CAR in commercial cell based therapies include the anti-BCMA CAR engineered in cells of idecabtagene vicleucel (ABECMA®) or ciltacabtagene autoleucel (CARVYKTI™).

CD38 (cluster of differentiation 38), also known as cyclic ADP ribose hydrolase is a glycoprotein found on the surface of many immune cells (white blood cells), in particular T-cells, including CD4+, CD8+, B lymphocytes and natural killer cells. CD38 also functions in cell adhesion, signal transduction and calcium signaling. Structural information about this protein can be found in the UniProtKB/Swiss-Prot database under reference P28907. In humans, the CD38 protein is encoded by the CD38 gene which located on chromosome 4. CD38 is a multifunctional ectoenzyme that catalyzes the synthesis and hydrolysis of cyclic ADP-ribose (cADPR) from NAD+ to ADP-ribose. These reaction products are deemed essential for the regulation of intracellular Ca2+. Also, loss of CD38 function was associated with impaired immune responses and metabolic disturbances (Malavasi F., et al. (2008). “Evolution and function of the ADP ribosyl cyclase/CD38 gene family in physiology and pathology”. Physiol. Rev. 88(3): 841-86). CD38 protein is a marker of HIV infection, leukemias, myelomas, solid tumors, type II diabetes mellitus and bone metabolism. CD38 expression as an important prognostic factor in B-cell chronic lymphocytic leukemia. Blood 98:181-186). In some embodiments, an anti-CD38 CAR may comprise an anti-CD38 single-chain variable fragment (scFv) specific for CD38, followed by a spacer and transmembrane domain that is fused to an intracellular co-signaling domain (e.g., a CD28 or 4-1BB) and a CD3zeta signaling domain.

In some embodiments, the extracellular binding domain of the CD38 CAR may comprise the heavy chain variable region (V_{H}) set forth in SEQ ID NO:46 or SEQ ID NO:47 and the light chain variable region (V_{L}) set forth in SEQ ID NO:48 or SEQ ID NO: 49. In some embodiments, the linker separating the VH and VL in the scFv is a GS linker, such as set forth in SEQ ID NO: 56. In some embodiments, the linker separating the VH and VL in the scFv is the Whitlow linker set forth in SEQ ID NO:55. In some embodiments, the intracellular signaling domain contains a 4-1BB costimulatory signaling domain and a CD3 zeta signaling domain, such as any described herein. In some embodiments, the intracellular signaling domain contains a CD28 costimulatory signaling domain and a CD3 zeta signaling domain, such as any described herein. In some embodiments, it is understood the CAR includes any sequences that exhibit some sequence variation to any of the above or described SEQ ID NOS, such as at least 85%, 90%, 95% or more sequence identity thereto, and retain binding to CD38 and intracellular signaling and cytotoxic activity.

B. Immunomodulator (e.g. Cytokine)

In provided embodiments, the engineered g-NK cells, or a plurality of g-NK cells, are engineered to express a heterologous immunomodulatory, such as an exogenous cytokine, e.g. an interleukin. In some embodiments, the heterologous nucleic acid encoding the immunomodulator is stably integrated into the genome of the g-NK cell. In other embodiments, the heterologous nucleic acid encoding the immunomodulator is transiently expressed. In some embodiments, the immunomodulator is an immunosuppressant. In other embodiments, the immunomodulator is an immunoactivator. In some embodiments, the immunoactivator is a cytokine.

In provided embodiments, the engineered NK cells express a heterologous cytokine or a functional portion thereof. According to provided embodiments, the NK cells are engineered, in some embodiments, to express a cytokine in a secreted form, while in some embodiments, the cytokine is membrane bound. In some embodiments, the heterologous cytokine or functional portion thereof is secretable from the cell. In some embodiments, the heterologous cytokine or functional portion thereof is expressed as a membrane bound protein on the surface of the cell.

Cytokines are a broad class of proteins that play an important role in cell signaling, particularly in the context of the immune system. Cytokines have been shown to play a role in autocrine, paracrine, and endocrine signaling as immunomodulating agents. Cytokines may function as immunoactivators, stimulating an immune-mediated response, or as immunosuppressants, damping down immune-mediated responses. Cytokines include chemokines, interferons, interleukins, lymphokines, and tumor necrosis factors, but generally not hormones or growth factors.

In some embodiments, the cytokine is an interleukin. Interleukins are a group of cytokines that are generally secreted proteins and signal molecules that mediate a broad range of immune responses. For example, Interleukin (IL)-2 plays a role in regulating the activities of white blood cells, while Interleukin (IL)-15 plays a major role in the development of inflammatory and protective immune responses to microbial invaders and parasites through modulating the activities of cells of both the innate and adaptive immune systems. In some embodiments, one or more activities of NK cells, including g-NK cells as provided, are regulated by IL-2, IL-21 and/or IL-15 or another cytokine as described.

As cytokines are necessary for NK cell activity, typical methods involve administering exogenous cytokines to a subject in combination with an NK cell therapy as exogenous cytokine support. However, in some aspects, the administration of exogenous cytokines may lead to a risk of systemic toxicity, particularly as can occur with high dose administration of certain cytokines. In provided embodiments, engineering the NK cells with secretable cytokines or membrane-bound cytokines provides a local source of the cytokines to the NK cells while avoiding or reducing risks of systemic toxicities.

In some embodiments of provided engineered cells, an interleukin or a functional portion thereof is introduced into a g-NK cell or a population of g-NK cells. In some embodiments, the interleukin includes a cytokine produced by immune cells such as lymphocytes, monocytes or macrophages. In some embodiments, the cytokine is an immune activating cytokine (also called an immunoactivator) that can be used to induce NK cells, such as to the promotion of NK cell survival, activation and/or proliferation. For instance, certain cytokines, such as IL-15 or IL-21, may prevent or reduce NK cells from undergoing senescence, such as by improving their ability to expand ex vivo or in vivo. In some embodiments, the interleukin or functional portion thereof is a partial or full peptide of one or more of IL-2, IL-4, IL-6, IL-7, IL-9, IL-10, IL-11, IL-12, IL-15, IL-18, or IL-21. In some embodiments, the cytokine is IL-2, IL-7, IL-12, IL-15, IL-18, IL-21, Flt3-L, SCF, or IL-7. In some embodiments, the cytokine is IL-2 or a functional portion thereof. In some embodiments, the cytokine is IL-12 or a functional portion thereof. In some embodiments the cytokine is IL-15 or a functional portion thereof. In some embodiments, the cytokine is IL-21 or a functional portion thereof. In some embodiments, the cytokine may be introduced with the respective receptor for the cytokine. In some embodiments, the steps of engineering a heterologous cytokine into the engineered cells permits cytokine signaling, thereby maintaining or improving cell growth, proliferation, expansion and/or effector function of the NK cells but with reduced risk of cytokine toxicities. In some embodiments, the introduced cytokine, or in some cases also its respective cytokine receptor, are expressed on the cell surface. In some embodiments, the cytokine signaling is constitutively activated. In some embodiments, the activation of the cytokine signaling is inducible. In some embodiments, the activation of the cytokine signaling is transient or temporal.

Exemplary secretable and membrane-bound (mb) cytokines are known as described, for example, in patent publication Nos. US2017/0073638; US2020/0199532, US 2021/0024959; and PCT patent publication Nos. WO2015174928, WO 2019/126748, WO 2019/191495, WO2020056045, WO2021021907, WO 2021/011919, WO 2021/062281, any of which can be used in the provided engineered cells.

In some embodiments, the cytokine is IL-15 or a functional portion thereof. IL-15 is a cytokine that regulates NK cell activation and proliferation. In some cases, IL-15 and IL-12 share similar biological activities. For instance, IL-15 and IL-2 bind common receptor subunits, and may compete for the same receptor. In some embodiments, IL-15 induces the activation of JAK kinases, as well as the phosphorylation and activation of transcription activators STAT3, STAT5, and STAT6. In some embodiments, IL-15 promotes or regulates one or more functional activities of NK cells, such as the promotion of NK cell survival, regulation of NK cell and T cell activation and proliferation as well as the support of NK cell development from hematopoietic stem cells. In some embodiments, a functional portion is a portion of IL-15 (e.g. containing a truncated contiguous sequence of amino acids of full-length IL-15) that retains one or more functions of full length or mature IL-15, such as the promotion of NK cell survival, regulation of NK cell and T cell activation and proliferation as well as the support of NK cell development from hematopoietic stem cells. All or a functional portion of IL-15 can be expressed as a membrane-bound polypeptide and/or as a secreted polypeptide.

As will be appreciated by those of skill in the art, the sequence of a variety of IL-15 molecules are known in the art. In one aspect, the IL-15 is a wild type IL-15. In some aspects, the IL-15 is a mammalian IL-15 (e.g., Homo sapiens interleukin 15 (IL15), transcript variant 3, mRNA, NCBI Reference Sequence: NM_000585.4; Canis lupus familiaris interleukin 15 (IL 15), mRNA, NCBI Reference Sequence: NM_001197188.1; Felis catus interleukin 15 (IL15), mRNA, NCBI Reference Sequence: NM_001009207.1). Examples of “mammalian” or “mammals” include primates (e.g., human), canines, felines, rodents, porcine, ruminants, and the like. Specific examples include humans, dogs, cats, horses, cows, sheep, goats, rabbits, guinea pigs, rats and mice. In a particular aspect, the mammalian IL-15 is a human IL-15. Human IL-15 amino acid sequences include, for example, Genbank Accession Nos: NR_751915.1, NP_000576.1, AA100963.1, AA100964.1, AA100962.1, CAA71044.1, AAH18149.1, AAB97518.1, CAA63914.1, and CAA63913.1.

In some embodiments, the engineered NK cell comprises a heterologous nucleotide sequence encoding IL-15. In some embodiments, the IL-15 nucleotide sequence is set forth in SEQ ID NO:9 or is a sequence that has at least or at least about 85%, at least or at least about 90%, at least or at least about 95%, or at least or at least about 98% sequence identity to SEQ ID NO:9. In some embodiments, the IL-15 is expressed by the cell in a mature form lacking the signal peptide sequence and in some cases also lacking the propeptide sequence. In some embodiments, the IL-15 has the sequence of amino acids set forth in SEQ ID NO:2 or a sequence that has at least or at least about 85%, at least or at least about 90%, at least or at least about 95%, or at least or at least about 98% sequence identity to SEQ ID NO:2.

In some embodiments, the IL-15 molecule is a variant of human IL-5, e.g., having one or more amino acid alterations, e.g., substitutions, to the human IL-15 amino acid sequence. In some embodiments, the IL-15 variant comprises, or consists of, a mutation at position 45, 51, 52, or 72, e.g., as described in US 2016/0184399. In some embodiments, the IL-15 variant comprises, or consists of, an N, S or L to one of D, E, A, Y or P substitution. In some embodiments, the mutation is chosen from L45D, L45E, S51D, L52D, N72D, N72E, N72A, N72S, N72Y, or N72P (in reference to the sequence of human IL-15, SEQ ID NO: 2).

In embodiments, the IL-15 molecule comprises an IL-15 variant, e.g., a human IL-15 polypeptide having one or more amino acid substitutions. In some embodiments, the IL-15 molecule comprises a substitution at position 72, e.g., an N to D substitution. In one embodiment, the IL-15 molecule is an IL-15 polypeptide of SEQ ID NO: 2 into which is contained the amino acid substitution N72D or is an amino acid sequence having at least 80%, 85%, 90%, 95%, 96%, 97%, 98%, or 99% identity thereto, which has IL-15Ra binding activity.

In some embodiments, the cytokine is IL-2 or a functional portion thereof. In some embodiments, IL-2 is a member of a cytokine family that also includes IL-4, IL-7, IL-9, IL-15 and IL-21. IL-2 signals through a receptor complex consisting of three chains, termed alpha, beta and gamma. The gamma chain is shared by all members of this family of cytokine receptors. IL-2, which similar to IL-15, facilitates production of immunoglobulins made by B cells and induces the differentiation and proliferation of NK cells. Primary differences between IL-2 and IL-15 are found in adaptive immune responses. For example, IL-2 is necessary for adaptive immunity to foreign pathogens, as it is the basis for the development of immunological memory. On the other hand, IL-15 is necessary for maintaining highly specific T cell responses by supporting the survival of CD8 memory T cells. All or a functional portion of IL-2 can be expressed as a membrane-bound polypeptide and/or as a secreted polypeptide. As will be appreciated by those of skill in the art, the sequence of a variety of IL-2 molecules are known in the art. In one aspect, the IL-2 is a wild type IL-2. In some aspects, the IL-2 is a mammalian IL-2. In some embodiments, the IL-2 is a human IL-2.

In some embodiments, the engineered NK cell comprises a heterologous nucleotide sequence encoding IL-2. In some embodiments, the IL-2 is expressed by the cell in a mature form lacking the signal peptide sequence and in some cases also lacking the propeptide sequence. In some embodiments, the IL-2 has the sequence of amino acids set forth in SEQ ID NO:1 or a sequence that has at least or at least about 85%, at least or at least about 90%, at least or at least about 95%, or at least or at least about 98% sequence identity to SEQ ID NO:1.

In some embodiments, the cytokine is IL-21 or a functional portion thereof. IL-21 binds to the IL-21 receptor (IL-21 R) and co-receptor, the common gamma chain (CD 132), The IL-21 receptor has been identified on NK cells, T cells and B cell indicating IL-21 acts on hematopoietic lineage cells, in particular lymphoid progenitor cells and lymphoid cells. IL-21 has been shown to be a potent modulator of cytotoxic T cells and NK cells. (Parrish-Novak, et al. Nature 408:57-63, 2000; Parrish-Novak, et al., J. Leuk. Bio. 72:856-863, 202: Collins et al., Immunol. Res. 28:131-140, 2003; Brady, et al. J. Immunol. 172:2048-58, 2004.) In murine studies, IL-21 potentiates the maturation and effector function of NK cells (Kasaian et al., Immunity 16:559-569, 2002).

As will be appreciated by those of skill in the art, the sequence of a variety of IL-21 molecules are known in the art. In one aspect, the IL-21 is a wild type IL-21. In some aspects, the IL-21 is a mammalian IL-21. In an embodiment, the IL-21 sequence is a human IL-21 sequence. Human IL-21 amino acid sequences include, for example, Genbank Accession Nos: AAU88182.1, EAX05226.1, CAI94500.1, CAJ47524.1, CAL81203.1, CAN87399.1, CAS03522.1, CAV33288.1, CBE74752.1, CBI70418.1, CBI85469.1, CBI85472.1, CBL93962.1, CCA63962.1, AAG29348.1, AAH66258.1, AAH66259.1, AAH66260.1, AAH66261.1, AAH66262.1, AAH69124.1, and ABG36529.1.

In some embodiments, the engineered NK cell comprises a heterologous nucleotide sequence encoding IL-21. In some embodiments, the IL-21 is expressed by the cell in a mature form lacking the signal peptide sequence and in some cases also lacking the propeptide sequence. In some embodiments, the IL-21 has the sequence of amino acids set forth in SEQ ID NO:3 or a sequence that has at least or at least about 85%, at least or at least about 90%, at least or at least about 95%, or at least or at least about 98% sequence identity to SEQ ID NO:3. In some embodiments, the IL-21 has the sequence of amino acids set forth in SEQ ID NO:4 or a sequence that has at least or at least about 85%, at least or at least about 90%, at least or at least about 95%, or at least or at least about 98% sequence identity to SEQ ID NO:4.

The cytokine (e.g., IL-2, IL-15, or IL-21) amino acid sequences may comprise any functional portion of mature cytokine, e.g. any functional portion of a mature, IL-2, mature, IL-15 or mature IL-21. The functional portion can be any portion comprising contiguous amino acids of the interleukin of which it is a part, provided that the functional portion specifically binds to the respective interleukin receptor. The term “functional portion” when used in reference to an interleukin refers to any part or fragment of the interleukin, which part or fragment retains the biological activity of the interleukin of which it is a part (the parent interleukin). Functional portions encompass, for example, those parts of an interleukin that retain the ability to specifically bind to the respective interleukin receptor, activate the downstream targets of the interleukin, and/or induce one or more of the differentiation, proliferation (or death) and activity of immune cells, e.g., NK cells, to a similar extent, the same extent, or to a higher extent, as the parent interleukin. The biological activity of the functional portion of the interleukin may be measured using assays known in the art. In reference to the parent interleukin, the functional portion can comprise, for instance, about 60%, about 70%, about 80%, about 90%, about 95%, or more, of the amino acid sequence of the parent mature interleukin.

Included in the scope of the cytokine or functional portion in accord with the provided embodiments are functional variants of the interleukins described herein. The term “functional variant” as used herein refers to an interleukin having substantial or significant sequence identity or similarity to a parent interleukin, which functional variant retains the biological activity of the interleukin of which it is a variant. Functional variants encompass, for example, those variants of the interleukin described herein (the parent interleukin) that retain the ability to specifically bind to the respective interleukin receptor, activate the downstream targets of the interleukin, and/or induce one or more of the differentiation, proliferation (or death) and activity of immune cells, e.g., NK cells, to a similar extent, the same extent, or to a higher extent, as the parent interleukin. In reference to the parent interleukin, the functional variant can, for instance, be at least about 80%, about 90%, about 95%, about 99% or more identical in amino acid sequence to the parent interleukin.

A functional variant can, for example, comprise the amino acid sequence of the parent interleukin with at least one conservative amino acid substitution. Alternatively or additionally, the functional variants can comprise the amino acid sequence of the parent interleukin with at least one non-conservative amino acid substitution. In some embodiments, the amino acid substitution, e.g. conservative or non-conservative amino acid substitution, does not interfere with or inhibit the biological activity of the functional variant as compared to the parental interleukin sequence. In some embodiments, the amino acid substitution, e.g. conservative or non-conservative amino acid substitution, may enhance the biological activity of the functional variant, such that the biological activity of the functional variant is increased as compared to the parent interleukin.

In some embodiments, the amino acid substitution(s) of the interleukin are conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, lie, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gin, Ser, Thr, Tyr, etc.), an amino acid with a beta-branched side-chain substituted for another amino acid with a beta-branched side-chain (e.g., lie, Thr, and Val), an amino acid with an aromatic side-chain substituted for another amino acid with an aromatic side chain (e.g., His, Phe, Trp, and Tyr), etc.

In some embodiments, all or a functional portion of a cytokine (e.g. IL-2, IL-15, IL-21 or a functional portion of any of the foregoing) can be expressed by a g-NK cell as a secreted polypeptide in a variety of ways. For example, all or a functional portion of the cytokine can be expressed within the NK cell and secreted from the NK cell. In some embodiments, a secretable cytokine does not contain a transmembrane domain.

In some embodiments, the cytokine is secretable from the engineered g-NK cell. In some embodiments, the secretable cytokine is constitutively expressed. In other embodiments, the secretable cytokine is transiently expressed. In some embodiments, the secretable cytokine is under an inducible promoter. In some embodiments, the secretable cytokine is IL-2 or a functional portion thereof. In some embodiments, the amino acid sequence of IL-2 is or comprises SEQ ID NO:1. In some embodiments, the secretable cytokine is IL-15 or a functional portion thereof. In some embodiments, the amino acid sequence of IL-15 is or comprises SEQ ID NO:2. In some embodiments, the secretable cytokine is IL-21 or a functional portion thereof. In some embodiments, the amino acid sequence of IL-21 is or comprises SEQ ID NO:3. In some embodiments, the g-NK cells are engineered with two or more secretable cytokines, such as a combination of two or more of IL-2, IL-15, and IL-21.

Although interleukins and other cytokines are generally secreted, they can also be membrane bound. When co-expressed with a CAR fusion protein, it is then possible to concentrate the immune-cell activating cytokine and the CAR fusion protein in close proximity to the target cell. When co-expressed in with a CAR fusion protein in a g-NK cell, the g-NK cells show an increase targeting and killing ability, thus representing an attractive and effective therapeutic agent. In some cases, a membrane-bound cytokine makes it possible to concentrate the immune-cell activating cytokine in close proximity to a target cell, for example, a cell that is targeted by a monoclonal antibody administered in combination with the g-NK cells as described herein.

In other embodiments, the cytokine is membrane-bound (mb). In some embodiments, the membrane-bound cytokine is constitutively expressed. In other embodiments, the membrane-bound cytokine is transiently expressed. In some embodiments, the membrane-bound cytokine is under an inducible promoter. In some embodiments, the membrane-bound cytokine is a membrane-bound IL-2 (mbIL-2). In some embodiments, the membrane-bound cytokine is a membrane-bound IL-15 (mbIL-15). In some embodiments, the membrane-bound cytokine is a membrane bound IL-21 (mbIL-21). In some embodiments, the g-NK cells are engineered with two or more membrane-bound cytokines, such as a combination of two or more of mbIL-2, mbIL-15, and mbIL-21. The membrane-bound cytokine can include any format of an interleukin cytokine (e.g., IL-2, IL-15 or IL-21) that is formatted in membrane bound form, such as any described herein.

In some embodiments, all or a functional portion of a cytokine (e.g. IL-2, IL-15, IL-21 or a functional portion of any of the foregoing) can be expressed by a g-NK cell as a membrane-bound cytokine in a variety of ways. In some embodiments, the cytokine or a functional portion thereof can be linked (e.g. conjugated or fused) directly or indirectly (e.g., ionic, non-ionic, covalent linkage) to the surface (e.g., at the surface, or within the membrane, of the NK cell) of the g-NK cell using any of a variety of linkers known in the art (Hermanson, G., Bioconjugate Techniques, Academic Press 1996). In some aspects, all or a functional portion of the cytokine is linked to all or a portion of a transmembrane protein. In one aspect, the NK cell expresses a fusion protein comprising all or a portion of the cytokine fused to all or a portion of a transmembrane protein. In some embodiments, the linker may be a peptide linker, such as a flexible linker. In some embodiments, the flexible linker comprises mainly glycine and serine residues. For example, the flexible linker may comprise one or more repeats of one or both of G4S and G3S (e.g., about 3 to about 15 or about 5 to about 12 repeats of G4S and G3S). In some embodiments, the linker is a cleavable linker, such as a furin cleavable sequence. Exemplary furin cleavage sequences are described in Duckert et al, Protein Engineering, Design & Selection, 17(1): 107-112 (2004) and U.S. Pat. No. 8,871,906, each of which is incorporated herein by reference.

In a particular aspect, the portion of the transmembrane protein comprises all or a portion of a transmembrane domain of the transmembrane protein. In some embodiments, the transmembrane protein may be any protein located at and/or within a membrane such as the phospholipid bilayer of a biological membrane (e.g., biomembranes such as the membrane of a cell). In some embodiments, the transmembrane domain is a domain of a transmembrane protein that is normally present within the membrane, particularly those that form channels and pores. In some embodiments, a transmembrane domain is a three-dimensional protein structure which is thermodynamically stable in a membrane (e.g., a membrane of a vesicle such as a cell). Examples of transmembrane domains include a single alpha helix, a stable complex of several transmembrane alpha helices, a transmembrane beta barrel, a beta-helix of gramicidin A, or any other structure. Transmembrane helices are usually about 20 amino acids in length.

Examples of transmembrane proteins include a receptor, a ligand, an immunoglobulin, a glycophorin or a combination thereof. Specific examples of transmembrane proteins include, but are not limited to, CD8α, CD4, CD3ε, CD3γ, CD3δ, CD3ζ, CD28, CD137, FcεRIγ, a T-cell receptor (TCR such as TCRα and/or TCRβ), a nicotinic acetylcholine receptor, a GABA receptor, or a combination thereof. Specific examples of immunoglobulins include IgG, IgA, IgM, IgE, IgD or a combination thereof. Specific examples of glycophorin include glycophorin A, glycophorin D or a combination thereof.

In some embodiments, the transmembrane domain is a CD28 transmembrane domain. An exemplary sequence of a CD28 transmembrane domain is set forth in SEQ ID NO:10.

(SEQ ID NO: 10)
IEVMYPPPYLDNEKSNGTIIHVKGKHLCPSPLFPGPSKPFWVLVVVGGV
LACYSLLVTVAFIIFWVR

In some embodiments, the transmembrane domain is a CD8 transmembrane domain. An exemplary sequence of a CD8 transmembrane domain is set forth in SEQ ID NO:11.

(SEQ ID NO: 11)
TTTPAPRPPTPAPTIASQPLSLRPEACRPAAGGAVHTRGLDFACDIYIW
APLAGTCGVLLLSLVITLYC

In some embodiments, the transmembrane domain is a CD4 transmembrane domain. An exemplary sequence of a CD4 transmembrane domain is set forth in SEQ ID NO:15.

(SEQ ID NO: 15)
MALIVLGGVAGLLLFIGLGIFF

In some embodiments, all or a functional portion of a cytokine e.g. IL-2, IL-15, IL-21 or a functional portion of any of the foregoing) can be linked to other components such as a signal peptide, a leader sequence, a secretory signal, a label (e.g., a reporter gene), or any combination thereof.

In some embodiments, the nucleic acid sequence encoding all or a functional portion of a cytokine (e.g. IL-2, IL-15, IL-21 or a functional portion of any of the foregoing) is replaced with a nucleic acid sequence encoding a signal peptide from a heterologous protein. The heterologous protein can be, for example, CD8α, CD28, tissue plasminogen activator (tPA), growth hormone, granulocyte-macrophage colony stimulating factor (GM-CSF), GM-CSF receptor (GM-CSFRa), or an immunoglobulin (e.g., IgE or IgK).

In some embodiments, all or a functional portion of a cytokine e.g. IL-2, IL-15, IL-21 or a functional portion of any of the foregoing) is fused to a signal peptide of CD8α. An exemplary CD8α signal peptide is set forth in SEQ ID NO:12. In some embodiments, all or a functional portion of a cytokine (e.g. IL-15 or a functional portion thereof, IL-2 or a functional portion thereof, or IL-21 or a functional portion thereof) is fused to a signal peptide of GM-CSFRa (SEQ ID NO:13). An exemplary GM-CSFRa signal peptide is set forth in SEQ ID NO:13. An exemplary IgK signal peptide is set forth in SEQ ID NO:14. An exemplary IgK signal peptide is set forth in SEQ ID NO: 43.

In some embodiments, all or a functional portion of a cytokine (e.g. IL-2, IL-15, IL-21 or a functional portion of any of the foregoing) is fused to a signal peptide of CD8α and all or a portion of a transmembrane domain of CD8α. In some embodiments, the heterologous cytokine is a membrane bound IL-15 set forth in SEQ ID NO:7 or a sequence that has at least or at least about 85%, at least or at least about 90%, at least or at least about 95%, or at least or at least about 98% sequence identity to SEQ ID NO:7. In some embodiments, the heterologous cytokine is a membrane bound IL-15 set forth in SEQ ID NO:8 or a sequence that has at least or at least about 85%, at least or at least about 90%, at least or at least about 95%, or at least or at least about 98% sequence identity to SEQ ID NO:8.

In some embodiments, all or a functional portion of a cytokine (e.g. IL-2, IL-15, IL-21 or a functional portion of any of the foregoing) is fused to an Fc region of an immunoglobulin to generate a bivalent cytokine. In some embodiments, the cytokine-Fc fusion protein may be further linked to a transmembrane domain for expression as a membrane-bound cytokine.

In some embodiments, the heterologous cytokine is a membrane bound IL-15 set forth in SEQ ID NO:5 or a sequence that has at least or at least about 85%, at least or at least about 90%, at least or at least about 95%, or at least or at least about 98% sequence identity to SEQ ID NO:5.

In some embodiments, the heterologous cytokine is a membrane bound IL-21 set forth in SEQ ID NO:6 or a sequence that has at least or at least about 85%, at least or at least about 90%, at least or at least about 95%, or at least or at least about 98% sequence identity to SEQ ID NO:6.

In some embodiments, the IL-15 is engineered into the cells with IL-15 Receptor alpha (IL15RA). IL15RA specifically binds IL-15 with very high affinity, and is capable of binding IL-15 independent of other subunits. In some aspects, this property allows IL-15 to be produced by one cell, endocytosed by another cell, and then presented to a third cell. In some embodiments, the g-NK cells expresses a heterologous (e.g. exogenous) IL-15/IL-15Ra. In some embodiments, the g-NK cell is engineered with an IL-15/IL-15R fusion protein. In some embodiments, the g-NK cell is engineered with a single-chain IL-15/IL-15R fusion protein. In some embodiments, the IL-15/IL-15Ra is expressed as a membrane-bound IL-15.IL 15Ra complex (e.g. Imamura et al., Blood, 2014 124(7):108 and Hurton L V et al., PNAS, 2016). In some embodiments, the exogenous IL-15/IL-15Ra is secretable and is expressed as a soluble IL15Ra.IL15 complex (e.g. Mortier E et al., JBC 2006; Bessard A, Mol. Cancer Ther., 2009; and Desbois M, J. Immunol., 2016). In some embodiments, the provided engineered g-NK cells expresses a membrane-bound IL 15/IL 15Ra complex and a soluble (secretable) IL 15Ra/IL15 complex. In some embodiments, the engineered g-NK cell expresses a membrane-bound from of IL15.IL15Ra complex with a cleavable linker.

### C. Polynucleotides

In some embodiments, provided herein is a polynucleotide having a nucleic acid sequence encoding an antigen receptor, such as a chimeric antigen receptor, including any of the provided chimeric antigen receptors. In some embodiments, provided herein is a polynucleotide having a nucleic acid sequence encoding any of the provided immunomodulators, such as cytokines, including a secretable or membrane-bound cytokine.

In some embodiments, the nucleic acid encoding an antigen receptor, such as a chimeric antigen receptor, and the nucleic acid encoding the immunomodulator, such as a cytokine, including a secretable or membrane-bound cytokine are provided as separate polynucleotides.

In some embodiments, the polynucleotide comprises a nucleic acid sequence encoding an antigen receptor, such as a chimeric antigen receptor, and a nucleic acid encoding the immunomodulator, such as a cytokine, including a secretable or membrane-bound cytokine. Thus, in some aspects the nucleic acid sequences are provided as part of the same polynucleotide. For instance, provided embodiments include polynucleotides in which engineered components are encoded by a polynucleotide that includes one or more protease cleavage site, for example a self-cleaving peptide, such as a T2A, a P2A, an E2A, or a F2A. Such sites are recognized and cleaved by a proteinase, which can result in separation (and separate expression) of the various component parts (e.g. cytokine and CAR) encoded by a polynucleotide engineered into an NK cell. As a result, depending on the embodiment, the various constituent parts of an engineered components can be delivered to an NK cell in a single vector or by multiple vectors.

Also provided herein are vehicles encodings any of the provided polynucleotides, such as for delivery of the polynucleotides to a cell, e.g. g-NK cell. In some embodiments, the vehicle is a vector, such as a viral vector or a non-viral vector. In some embodiments, the vehicle is a viral vector that is a lentiviral vector. In some embodiments, the vehicle is a liposome. In some embodiments, the vehicle is a lipid nanoparticle. Other vehicles, including vectors or non-vector delivery vehicles include those known to a skilled artisan, including any described below.

In some embodiments, the polynucleotides are engineered into a g-NK cells, or a composition containing a plurality of g-NK cells, in accord with the provided methods. Exemplary methods of engineering NK cells are described below.

### D. Methods of Delivery of Heterologous Agents

Disclosed herein is a method of producing a genetically engineered g-NK cell, comprising introducing into a g-NK cell a nucleic acid encoding a CAR, thereby producing a genetically engineered g-NK cell.

Disclosed herein is a method of producing a genetically engineered g-NK cell, comprising introducing into a g-NK cell a nucleic acid encoding an immunomodulator (e.g. a secretable or soluble cytokine or membrane-bound cytokine), thereby producing a genetically engineered g-NK cell.

Disclosed herein is a method of producing a genetically engineered g-NK cell, comprising (a) introducing into a g-NK cell a nucleic acid encoding a CAR, and (b) introducing into the g-NK cell a nucleic acid encoding an immunomodulator (e.g. a secretable or soluble cytokine or membrane-bound cytokine), thereby producing a genetically engineered g-NK cell, wherein steps (a) and (b) are carried out simultaneously or sequentially in any order.

The nucleic acid that is introduced into the g-NK cell may be introduced for stable integration into the genome or for transient expression. Stable integration versus transient expression may be selected based off of various factors including, but not limited to, the ability of a particular nucleic acid to be efficiently integrated into the host genome or the content of the nucleic acid and its half-life.

In some embodiments, introducing the heterologous agent(s) into the g-NK cells (e.g. antigen receptor, such as a CAR and/or an immunomodulatory agent, such as a cytokine as described) may be carried out in a method that enriches for g-NK cell subset from a starting sample of NK cells. In some embodiments, introducing the antigen receptor, such as a CAR, may be carried out in a method that enriches for g-NK cell subset from a starting sample of NK cells. In some embodiments, introducing the immunomodulator, such as a cytokine, may be carried out in a method that enriches for g-NK cell subset from a starting sample of NK cells. In some embodiments, introducing the antigen receptor, such as a CAR, and the immunomodulator, such as a cytokine, may be carried out in a method that enriches for g-NK cell subset from a starting sample of NK cells. Thus, it is understood that the provided methods do not require specifically engineering only g-NK cells that have been selected for NK cells that are deficient in the FcRγ chain (or only that have been selected or identified by a g-NK surrogate marker profile), but may involve engineering of cells of a composition of NK cells that are to be, or that have been, preferentially expanded or enriched in g-NK cells. As such, the final composition of cells that are enriched in g-NK cells include g-NK cells introduced with the heterologous antigen receptor (e.g. CAR), the heterologous immunomodulator (e.g. secretable or membrane-bound interleukin, such as IL-15 or IL-21), or the heterologous antigen receptor (e.g. CAR) and the heterologous immunomodulator, such as cytokine (e.g. secretable or membrane-bound interleukin, such as IL-15 or IL-21). Exemplary methods for preparing and expanding a composition enriched in g-NK cells is provided in Section V.

In some embodiments, the introducing of the heterologous agent(s) (e.g. antigen receptor, such as a CAR and/or an immunomodulatory agent, such as a cytokine as described) may take place at any suitable time during the methods of expanding the g-NK cells, such as described in Section V. In some embodiments, the introducing of the antigen receptor, such as a CAR, may take place at any suitable time during the methods of expanding the g-NK cells. In some embodiments, the introducing of the immunomodulator, such as a cytokine, may take place at any suitable time during the methods of expanding the g-NK cells. In some embodiments, the introducing of the CAR and the immunomodulator may take place at any suitable time during the methods of expanding the g-NK cells. In some embodiments, the introducing is carried out after the selection of cells from a subject (e.g. selecting or enriching cells that are CD3^{neg}CD57^{pos}) and prior to incubating or culturing the selected or enriched cells with feeder cells (e.g. HLA-E-expressing feeder cells) for proliferation or expansion of the NK cells. In some embodiments, the introducing is carried out after the incubation or culture with the feeder cells (e.g. HLA-E-expressing feeder cells) and thus after selected or enriched cells have proliferated or expanded. In some embodiments, the introducing is carried out sequentially, in any order, with the methods for gene editing as described herein.

In some embodiments, the period for expansion of the cells, such as described in Section V, is divided into a first expansion and a second expansion. In some embodiments, prior to the introduction (e.g. viral transduction), the selected cells from the biological sample are cultured under conditions for expansion for a first period of time, for example, for at or greater than about 6 hours, about 12 hours, about 18 hours, about 24 hours, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, or for any time between those listed, including endpoints. In some embodiments, after the first period of expansion, the expanded cells (e.g., NK cells) are introduced (e.g. transduced) with an engineered construct encoding one or more heterologous agent(s), such as a chimeric antigen receptor and/or an immunomodulatory agent, such as a cytokine as described. In some embodiments, after the first period of expansion, the expanded cells (e.g., NK cells) are introduced (e.g. transduced) with an engineered construct encoding one or more heterologous agent, such as a chimeric antigen receptor. In some embodiments, after the first period of expansion, the expanded cells (e.g., NK cells) are introduced (e.g. transduced) with an engineered construct encoding one or more heterologous agent, such as an immunomodulatory agent, such as a cytokine. In some embodiments, after the first period of expansion, the expanded cells (e.g., NK cells) are introduced (e.g. transduced) with an engineered construct encoding one or more heterologous agent(s), such as a chimeric antigen receptor and an immunomodulatory agent, such as a cytokine. After the introduction (e.g. viral transduction), the engineered cells are cultured for a second period of time, for example, for at or greater than about 6 hours, about 12 hours, about 18 hours, about 24 hours, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, or for any time between those listed, including endpoints.

Supplementation of the media with HLA-E expressing feeder cells and/or one or more stimulatory agents, such as IL 12 and/or IL21, can occur at any time during the culturing process. For example, one or more stimulatory agents can be added at the inception of culturing, for example at time point zero (e.g., inception of culture). The agent, or agents, can be added a second, third, fourth, fifth, or more times. Subsequent additions may, or may not, be at the same concentration as a prior addition. The interval between multiple additions can vary, for example a time interval of about 12 hours, about 24 hours, about 36 hours, about 48 hours, about 72 hours, or longer, and any time there between, including endpoints. If multiple additions of a stimulatory agent are used, the concentrations of a first supplemental addition can be at the same or a different concentration than the second (and/or any supplemental addition). For example, in several embodiments, the addition of a stimulatory agent over multiple time points can ramp up, ramp down, stay constant, or vary across multiple, non equivalent concentrations.

In some embodiments, the nucleic acid encoding the antigen receptor (e.g. CAR) is introduced under conditions for transient expression in the g-NK cell. In some embodiments, the nucleic acid encoding the immunomodulator (e.g. cytokine, such as secretable or soluble cytokine or membrane-bound cytokine) is introduced under conditions for transient expression in the g-NK cell. In some embodiments, the nucleic acid encoding the antigen receptor (e.g. CAR) and the nucleic acid encoding the immunomodulator (e.g. cytokine, such as secretable or soluble cytokine or membrane-bound cytokine) is introduced under conditions for transient expression in the g-NK cell. In some embodiments, methods for introducing a nucleic acid for transient expression includes any method that will result in a nucleic acid that may express its encoded content for a short period of time before being degraded.

In some embodiments, the nucleic acid encoding the antigen receptor (e.g. CAR) is introduced under conditions for stable expression in the g-NK cell. In some embodiments, the nucleic acid encoding the immunomodulator (e.g. cytokine, such as secretable or soluble cytokine or membrane-bound cytokine) is introduced under conditions for stable expression in the g-NK cell. In some embodiments, the nucleic acid encoding the antigen receptor (e.g. CAR) and the nucleic acid encoding the immunomodulator (e.g. cytokine, such as secretable or soluble cytokine or membrane-bound cytokine) is introduced under conditions for stable expression in the g-NK cell. In some embodiments, methods for introducing a nucleic acid for stable expression in a cell involves any method that results in stable integration of the nucleic acid into the genome of the cell, such that it may be propagated if the cell it has integrated into divides.

In some embodiments, one of the heterologous agents (e.g. one of the nucleic acid encoding the antigen receptor, such as a CAR, or the nucleic acid encoding the immunomodulator, such as a cytokine) is introduced into the cells by a method that results in transient expression in the cell and the other heterologous agent (e.g. the other of the nucleic acid encoding the antigen receptor, such as a CAR, or the nucleic acid encoding the immunomodulator, such as a cytokine) is introduced by a method that results in stable expression in a cell. In some embodiments, both of the heterologous agents (e.g. both the nucleic acid encoding the antigen receptor, such as a CAR, and the nucleic acid encoding the immunomodulator, such as a cytokine) are introduced into the cells by a method that results in transient expression in the cell. In some embodiments, both of the heterologous agents (e.g. both the nucleic acid encoding the antigen receptor, such as a CAR, and the nucleic acid encoding the immunomodulator, such as a cytokine) are introduced into the cells by a method that results in stable expression in the cell.

Methods of delivery of polynucleotides, and compositions containing the same, are known to a skilled artisan. It is within the level of a skilled artisan to choose an appropriate method for transient or stable expression in the cell.

In some embodiments, engineering of the NK cells can be accomplished by transducing a cell compositions with a polynucleotide encoding the heterologous agent(s) (e.g. CAR and/or immunomodulator, such as a cytokine), or a vector comprising said polynucleotide. In some embodiments, engineering of the NK cells can be accomplished by transducing a cell compositions with a polynucleotide encoding the antigen receptor, such as a CAR, or a vector comprising said polynucleotide. In some embodiments, engineering of the NK cells can be accomplished by transducing a cell compositions with a polynucleotide encoding the immunomodulator, such as a cytokine, or a vector comprising said polynucleotide. In some embodiments, engineering of the NK cells can be accomplished by transducing a cell compositions with polynucleotides encoding the antigen receptor, such as a CAR, and an immunomodulatory, such as a cytokine, or a vector comprising said polynucleotides. The vector may be a viral vector such as a lentiviral vector, a gamma-retroviral vector, a recombinant AAV, an adenoviral vector or an oncolytic viral vector. In other aspects, non-viral vectors for example, nanoparticles and liposomes may also be used for introducing and delivery of a polynucleotide encoding the heterologous agent(s) (e.g. CAR and/or immunomodulator, such as a cytokine) into the NK cell. In other aspects, non-viral vectors for example, nanoparticles and liposomes may also be used for introducing and delivery of a polynucleotide encoding the antigen receptor, such as a CAR, into the NK cell. In other aspects, non-viral vectors for example, nanoparticles and liposomes may also be used for introducing and delivery of a polynucleotide encoding the immunomodulator, such as a cytokine, into the NK cell. In other aspects, non-viral vectors for example, nanoparticles and liposomes may also be used for introducing and delivery of polynucleotides encoding the antigen receptor, such as a CAR, and an immunomodulator, such as a cytokine, into the NK cell.

In some embodiments, vectors that package a polynucleotide encoding a heterologous agent(s) (e.g. CAR and/or immunomodulator, such as a cytokine) may be used to deliver the packaged polynucleotides to a g-NK cell or to a composition or population of cells enriched in g-NK cells. In some embodiments, vectors that package a polynucleotide encoding an antigen receptor, such as a CAR, may be used to deliver the packaged polynucleotides to a g-NK cell or to a composition or population of cells enriched in g-NK cells. In some embodiments, vectors that package a polynucleotide encoding an immunomodulatory, such as a cytokine, may be used to deliver the packaged polynucleotides to a g-NK cell or to a composition or population of cells enriched in g-NK cells. In some embodiments, vectors that package a polynucleotide encoding an antigen receptor, such as a CAR, and an immunomodulatory, such as a cytokine, may be used to deliver the packaged polynucleotides to a g-NK cell or to a composition or population of cells enriched in g-NK cells. These vectors may be of any kind, including DNA vectors, RNA vectors, plasmids, viral vectors and particles. Viral vector technology is well known and described in Sambrook et al. (2001, Molecular Cloning: A Laboratory Manual, Cold Spring Harbor Laboratory, New York). Viruses, which are useful as vectors include, but are not limited to lentiviral vectors, adenoviral vectors, adeno-associated viral (AAV) vectors, herpes simplex viral vectors, retroviral vectors, oncolytic viruses, and the like.

In general, vectors contain an origin of replication functional in at least one organism, a promoter sequence and convenient restriction endonuclease site, and one or more selectable markers e.g. a drug resistance gene.

The promoter may include any DNA sequence recognized by transcription machinery of the cell, required to initiate specific transcription of the polynucleotide sequence. Vectors can comprise native or non-native promoters operably linked to the polynucleotides. The promoters selected may be strong, weak, constitutive, inducible, tissue specific, development stage-specific, and/or organism specific. One example of a suitable promoter is the immediate early cytomegalovirus (CMV) promoter sequence. This promoter sequence is a strong constitutive promoter sequence capable of driving high levels of expression of polynucleotide sequence that is operatively linked to it. Another example of a promoter is Elongation Growth Factor-1. Alpha (EF-1. alpha). Other constitutive promoters may also be used, including, but not limited to simian vims 40 (SV40), mouse mammary tumor virus (MMTV), human immunodeficiency vims (HIV), long terminal repeat (LTR), promoter, an avian leukemia vims promoter, an Epstein-Barr vims immediate early promoter, a Rous sarcoma vims promoter as well as human gene promoters including, but not limited to the phosphoglycerate kinase (PGK) promoter, actin promoter, the myosin promoter, the hemoglobin promoter, the Ubiquitin C (Ubc) promoter, the human U6 small nuclear protein promoter and the creatine kinase promoter. In some instances, inducible promoters such as but not limited to metallothionine promoter, glucocorticoid promoter, a progesterone promoter, and a tetracycline promoter may be used.

Additional promoter elements e.g. enhancers may be used to regulate the frequency of transcriptional initiation. Such regions may be located 10-100 base pairs upstream or downstream of the start site. In some instances, two or more promoter elements may be used to cooperatively or independently activate transcription.

In some embodiments, polynucleotides may be packaged into viral vectors or integrated into viral genomes allowing transient or stable expression of the polynucleotides. Viral vectors may include retroviral vectors including lentiviral vectors. In order to construct a retroviral vector, a polynucleotide molecule encoding a heterologous agent(s) (e.g. CAR and/or immunomodulator, such as a cytokine) is inserted into the viral genome in the place of certain viral sequences to produce a virus that is replication-defective. In order to construct a retroviral vector, a polynucleotide molecule encoding the antigen receptor, such as a CAR, is inserted into the viral genome in the place of certain viral sequences to produce a virus that is replication-defective. In order to construct a retroviral vector, a polynucleotide molecule encoding the immunomodulator, such as a cytokine, is inserted into the viral genome in the place of certain viral sequences to produce a virus that is replication-defective. In order to construct a retroviral vector, polynucleotide molecules encoding the antigen receptor, such as a CAR, and the immunomodulator, such as a cytokine, are inserted into the viral genome in the place of certain viral sequences to produce a virus that is replication-defective. The recombinant viral vector is then introduced into a packaging cell line containing the gag, pol, and env genes, but without the LTR and packaging components. The recombinant retroviral particles are secreted into the culture media, then collected, optionally concentrated, and used for gene transfer. Lentiviral vectors are especially preferred as they are capable of infecting both dividing and non-dividing cells.

In some embodiments, the polynucleotides encoding a heterologous agent(s) (e.g. CAR and/or immunomodulator, such as a cytokine) are incorporated into a viral vector for delivery by transduction. In some embodiments, the polynucleotides encoding the antigen receptor, such as a CAR, are incorporated into a viral vector for delivery by transduction. In some embodiments, the polynucleotides encoding the immunomodulator, such as a cytokine, are incorporated into a viral vector for delivery by transduction. In some embodiments, the polynucleotides encoding the antigen receptor, such as a CAR, and the immunomodulator, such as a cytokine, are incorporated into a viral vector for delivery by transduction. Viral transduction is a process whereby nucleic acids are deliberately introduced into eukaryotic cells through virus-mediated means.

In some embodiments, the viral vector is a lentiviral vector. Lentiviral vectors are particularly useful means for successful viral transduction as they permit stable expression of the gene contained within the delivered nucleic acid transcript. Lentiviral vectors express reverse transcriptase and integrase, two enzymes required for stable expression of the gene contained within the delivered nucleic acid transcript. Reverse transcriptase converts an RNA transcript into DNA, while integrase inserts and integrates the DNA into the genome of the target cell. Once the DNA has been integrated stably into the genome, it divides along with the host. The gene of interest contained within the integrated DNA may be expressed constitutively or it may be inducible. As part of the host cell genome, it may be subject to cellular regulation, including activation or repression, depending on a host of factors in the target cell.

Lentiviruses are subgroup of the Retroviridae family of viruses, named because reverse transcription of viral RNA genomes to DNA is required before integration into the host genome. As such, the most important features of lentiviral vehicles/particles are the integration of their genetic material into the genome of a target/host cell. Some examples of lentivirus include the Human Immunodeficiency Viruses: HIV-1 and HIV-2, the Simian Immunodeficiency Virus (SIV), feline immunodeficiency virus (FIV), bovine immunodeficiency virus (BIV), Jembrana Disease Virus (JDV), equine infectious anemia virus (EIAV), equine infectious anemia, virus, visna-maedi and caprine arthritis encephalitis virus (CAEV).

Typically, lentiviral particles making up the gene delivery vehicle are replication defective on their own (also referred to as “self-inactivating”). Lentiviruses are able to infect both dividing and non-dividing cells by virtue of the entry mechanism through the intact host nuclear envelope (Naldini L et al., Curr. Opin. Bioiecknol, 1998, 9: 457-463). Recombinant lentiviral vehicles/particles have been generated by multiply attenuating the HIV virulence genes, for example, the genes Env, Vif, Vpr, Vpu, Nef and Tat are deleted making the vector biologically safe. Correspondingly, lentiviral vehicles, for example, derived from HIV-1/HIV-2 can mediate the efficient delivery, integration and long-term expression of transgenes into non-dividing cells.

Lentiviral particles may be generated by co-expressing the virus packaging elements and the vector genome itself in a producer cell such as human HEK293T cells. These elements are usually provided in three (in second generation lentiviral systems) or four separate plasmids (in third generation lentiviral systems). The producer cells are co-transfected with plasmids that encode lentiviral components including the core (i.e. structural proteins) and enzymatic components of the virus, and the envelope protein(s) (referred to as the packaging systems), and a plasmid that encodes the genome including a foreign transgene, to be transferred to the target cell, the vehicle itself (also referred to as the transfer vector). In general, the plasmids or vectors are included in a producer cell line. The plasmids/vectors are introduced via transfection, transduction or infection into the producer cell line. Methods for transfection, transduction or infection are well known by those of skill in the art. As non-limiting example, the packaging and transfer constructs can be introduced into producer cell lines by calcium phosphate transfection, lipofection or electroporation, generally together with a dominant selectable marker, such as neomycin (neo), dihydrofolate reductase (DHFR), glutamine synthetase or adenosine deaminase (ADA), followed by selection in the presence of the appropriate drug and isolation of clones.

The producer cell produces recombinant viral particles that contain the foreign gene, for example, the polynucleotides encoding the heterologous agent(s) (e.g. CAR and/or immunomodulator, such as a cytokine). The producer cell produces recombinant viral particles that contain the foreign gene, for example, the polynucleotides encoding the antigen receptor, such as a CAR The producer cell produces recombinant viral particles that contain the foreign gene, for example, the polynucleotides encoding the immunomodulator, such as a cytokine. The producer cell produces recombinant viral particles that contain the foreign gene, for example, the polynucleotides encoding the antigen receptor, such as a CAR, and the immunomodulator, such as a cytokine. The recombinant viral particles are recovered from the culture media and titrated by standard methods used by those of skill in the art. The recombinant lentiviral vehicles can be used to infect target cells, such as g-NK cells or a composition or population of cells enriched in g-NK cells.

Cells that can be used to produce high-titer lentiviral particles may include, but are not limited to, HEK293T cells, 293G cells, STAR cells (Relander et al., Mol Ther. 2005, 11: 452-459), FreeStyle™ 293 Expression System (ThermoFisher, Waltham, MA), and other HEK293T-based producer cell lines (e.g., Stewart et al., Hum Gene Ther. 2011, 2, 2. (3):357-369; Lee et al, Biotechnol Bioeng, 2012, 10996): 1551-1560; Throm et al. Blood. 2009, 113(21): 5104-5110).

In some aspects, the envelope proteins may be heterologous envelope protein from other viruses, such as the G protein of vesicular stomatitis virus (VSV G) or baculoviral gp64 envelop proteins. The VSV-G glycoprotein may especially be chosen among species classified in the vesiculovirus genus: Carajas virus (CJSV), Chandipura virus (CHPV), Cocal virus (COCV), Isfahan virus (ISFV), Maraba virus (MARAV), Piry virus (PIRYV), Vesicular stomatitis Aiagoas virus (VSAV), Vesicular stomatitis Indiana virus (VSTV) and Vesicular stomatitis New Jersey virus (VSNJV) and/or stains provisionally classified in the vesiculovims genus as Grass carp rhabdovirus, BeAn 157575 virus (BeAn 157575), Boteke virus (BTKV), Calchaqui virus (CQFV), Eel virus American (EVA), Gray Lodge virus (GLOV), Jurona virus (JURY), Klamath virus (KLAVj. Kwatta virus (KWAV), La Joya virus (LJV), Malpais Spring virus (MSPV), Mount Elgon bat virus (MEB V), Ferine t virus (PERV), Pike fry rhabdovirus (PFRV), Porton virus (PORV), Radi virus (RADIV), Spring viremia of carp virus (SVCV), Tupaia virus (TUPV), Ulcerative disease rhabdovirus (UDRV) and Yug Bogdanovac virus (YBV). The gp64 or other baculoviral env protein can be derived from Autographa californica nucleopolyhedroviras (AcMNPV), Anagrapha falcifera nuclear polyhedrosis virus, Bombyx mori nuclear polyhedrosis virus, Choristoneura fiimiferana nucleopolyhedroviras, Orgyia pseudotsugata single capsid nuclear polyhedrosis virus, Epiphyas postvittana nucleopolyhedroviras, Hypharitria cunea nucleopolyhedroviras, Galleria mellonella nuclear polyhedrosis virus, Dhori virus, Thogoto virus, Antheraea pemyi nucleopolyhedroviras or Batken virus.

Additional elements provided in lentiviral particles may comprise retroviral LTR (long-terminal repeat) at either 5′ or 3′ terminus, a retroviral export element, optionally a lentiviral reverse response element (RRE), a promoter or active portion thereof, and a locus control region (LCR) or active portion thereof. Other elements include central polypurine tract (cPPT) sequence to improve transduction efficiency in non-dividing cells, Woodchuck Hepatitis Virus (WHP) Posttranscriptional Regulatory Element (WPRE) which enhances the expression of the transgene, and increases titer.

Methods for generating recombinant lentiviral particles are known to a skilled artisan, for example, U.S. Pat. Nos. 8,846,385; 7,745,179; 7,629,153; 7,575,924; 7,179,903; and 6,808,905. Lentivirus vectors used may be selected from, but are not limited to pLVX, pLenti, pLenti6, pLJMl, FUGW, pWPXL, pWPI, pLenti CMV puro DEST, pLJMl-EGFP, pULTRA, pInducer2Q, pHIV-EGFP, pCW57.1, pTRPE, pELPS, pRRL, and pLionII, Any known lentiviral vehicles may also be used (See, U.S. Pat. Nos. 9,260,725; 9,068,199; 9,023,646; 8,900,858; 8,748,169; 8,709,799; 8,420,104; 8,329,462; 8,076,106; 6,013,516; and 5,994,136; International Patent Publication NO.: WO2012079000).

Other retroviral vectors also may be used to package a heterologous agent(s) (e.g. CAR and/or immunomodulator, such as a cytokine) for delivery into g-NK cells or a composition or population of cells enriched in g-NK cells. Other retroviral vectors also may be used to package the antigen receptor, such as a CAR, for delivery into g-NK cells or a composition or population of cells enriched in g-NK cells. Other retroviral vectors also may be used to package the immunomodulator, such as a cytokine, for delivery into g-NK cells or a composition or population of cells enriched in g-NK cells. Other retroviral vectors also may be used to package the antigen receptor, such as a CAR, and the immunomodulator, such as a cytokine, for delivery into g-NK cells or a composition or population of cells enriched in g-NK cells. Retroviral vectors (RVs) allow the permanent integration of a transgene in target cells. In addition to lentiviral vectors based on complex HIV-1/2, retroviral vectors based on simple gamma-retroviruses have been widely used to deliver therapeutic genes and demonstrated clinically as one of the most efficient and powerful gene delivery systems capable of transducing a broad range of cell types. Example species of Gamma retroviruses include the murine leukemia viruses (MLVs) and the feline leukemia viruses (FeLV).

In some embodiments, gamma-retro viral vectors derived from a mammalian gamma-retrovirus such as murine leukemia viruses (MLVs), are recombinant. The MLV families of gamma retroviruses include the ecotropic, amphotropic, xenotropic and polytropic subfamilies. Ecotropic viruses are able to infect only murine cells using mCAT-1 receptor. Examples of ecotropic viruses are Moloney MLV and AKV. Amphotropic viruses infect murine, human and other species through the Pit-2 receptor. One example of an amphotropic virus is the 4070A virus. Xenotropic and polytropic viruses utilize the same (Xprl) receptor, but differ in their species tropism. Xenotropic viruses such as NZB-9-1 infect human and other species but not murine species, whereas polytropic viruses such as focus-forming viruses (MCF) infect murine, human and other species.

Gamma-retroviral vectors may be produced in packaging cells by co-transfecting the cells with several plasmids including one encoding the retroviral structural and enzymatic (gag-pol) polyprotein, one encoding the envelope (env) protein, and one encoding the vector mRNA comprising polynucleotide encoding the heterologous agent(s) (e.g. CAR and/or immunomodulator, such as a cytokine) that is to be packaged in newly formed viral particles. Gamma-retroviral vectors may be produced in packaging cells by co-transfecting the cells with several plasmids including one encoding the retroviral structural and enzymatic (gag-pol) polyprotein, one encoding the envelope (env) protein, and one encoding the vector mRNA comprising polynucleotide encoding the antigen receptor, such as a CAR, that is to be packaged in newly formed viral particles. Gamma-retroviral vectors may be produced in packaging cells by co-transfecting the cells with several plasmids including one encoding the retroviral structural and enzymatic (gag-pol) polyprotein, one encoding the envelope (env) protein, and one encoding the vector mRNA comprising polynucleotide encoding the immunomodulator, such as a cytokine, that is to be packaged in newly formed viral particles. Gamma-retroviral vectors may be produced in packaging cells by co-transfecting the cells with several plasmids including one encoding the retroviral structural and enzymatic (gag-pol) polyprotein, one encoding the envelope (env) protein, and one encoding the vector mRNA comprising polynucleotide encoding the antigen receptor, such as a CAR, and the immunomodulator, such as a cytokine, that is to be packaged in newly formed viral particles.

In some aspects, the recombinant gamma-retroviral vectors are pseudotyped with envelope proteins from other viruses. Envelope glycoproteins are incorporated in the outer lipid layer of the viral particles which can increase/alter the cell tropism. Exemplary envelope proteins include the gibbon ape leukemia vims envelope protein (GALV) or vesicular stomatitis virus G protein (VSV-G), or Simian endogenous retrovirus envelope protein, or Measles Virus H and F proteins, or Human immunodeficiency virus gp120 envelope protein, or cocal vesiculovirus envelope protein (See, e.g., U.S. application publication NO.: 2012/164118). In other aspects, envelope glycoproteins may be genetically modified to incorporate targeting/binding ligands into gamma-retroviral vectors, binding ligands including, but not limited to, peptide ligands, single chain antibodies and growth factors (Waehier et aL, Nat. Rev. Genet. 2007, 8(8):573-587). These engineered glycoproteins can retarget vectors to cells expressing their corresponding target moieties. In other aspects, a “molecular bridge” may be introduced to direct vectors to specific cells. The molecular bridge has dual specificities: one end can recognize viral glycoproteins, and the other end can bind to the molecular determinant on the target cell. Such molecular bridges, for example ligand-receptor, avidin-biotin, and chemical conjugations, monoclonal antibodies and engineered fusogenic proteins, can direct the attachment of viral vectors to target cells for transduction (Yang et al., Biotechnol Bioeng., 2008, 101(2): 357-368; and Maetzig et al, Viruses, 2011, 3, 677-713).

In some embodiments, the recombinant gamma-retroviral vectors are self-inactivating (SIN) gammaretroviral vectors. The vectors may be replication incompetent. SIN vectors may harbor a deletion within the 3′ U3 region initially comprising enhancer/promoter activity. Furthermore, the 5′ U3 region may be replaced with strong promoters (needed in the packaging cell line) derived from Cytomegalovirus or RSV, or an internal promoter of choice, and/or an enhancer element. The choice of the internal promoters may be made according to specific requirements of gene expression needed for a particular purpose.

In some embodiments, polynucleotides encoding the heterologous agent(s) (e.g. CAR and/or immunomodulator, such as a cytokine) are inserted within the recombinant viral genome. In some embodiments, polynucleotides encoding the antigen receptor, such as a CAR, are inserted within the recombinant viral genome. In some embodiments, polynucleotides encoding the immunomodulator, such as a cytokine, are inserted within the recombinant viral genome. In some embodiments, polynucleotides encoding the antigen receptor, such as a CAR, and the immunomodulator, such as a cytokine, are inserted within the recombinant viral genome. The other components of the viral mRNA of a recombinant gamma-retroviral vector may be modified by insertion or removal of naturally occurring sequences (e.g., insertion of an IRES, insertion of a heterologous polynucleotide encoding a polypeptide or inhibitory nucleic acid of interest, shuffling of a more effective promoter from a different retrovirus or virus in place of the wild-type promoter and the like). In some examples, the recombinant gamma-retroviral vectors may comprise modified packaging signal, and/or primer binding site (PBS), and/or 5′-enhancer/promoter elements in the U3-region of the 5′-long terminal repeat (LTR), and/or 3′-SIN elements modified in the US-region of the 3-LTR. These modifications may increase the titers and the ability of infection. Gamma retroviral vectors suitable for delivering the heterologous agent(s) (e.g. CAR and/or immunomodulator, such as a cytokine) may be selected from those disclosed in U.S. Pat. Nos. 8,828,718; 7,585,676; 7,351,585; U.S. application publication NO.: US2007/048285; PCT application publication NOs.: WO2010/113037; WO2014/121005; WO2015/056014; and EP Pat, NOs.: EP1757702; EP1757703).

In some embodiments, polynucleotides encoding the heterologous agent(s) (e.g. CAR and/or immunomodulator, such as a cytokine) may be packaged into recombinant adeno-associated viral (rAAV) vectors. In some embodiments, polynucleotides encoding the antigen receptor, such as a CAR, may be packaged into recombinant adeno-associated viral (rAAV) vectors. In some embodiments, polynucleotides encoding the immunomodulator, such as a cytokine, may be packaged into recombinant adeno-associated viral (rAAV) vectors. In some embodiments, polynucleotides encoding the antigen receptor, such as a CAR, and the immunomodulator, such as a cytokine, may be packaged into recombinant adeno-associated viral (rAAV) vectors. Such vectors or viral particles may be designed to utilize any of the known serotype capsids or combinations of serotype capsids. The serotype capsids may include capsids from any identified AAV serotypes and variants thereof, for example, AAV1, AAV2, AAV2G9, AAV3, AAV4, AAV4-4, AAV5, AAV6, AAV7, AAV8, AAV9, AAV10, AAV11, AAV12 and AAVrh10. In some embodiments, the AAV serotype may be or have a sequence as described in United States Publication No. US20030138772; Pulicherla et al. Molecular Therapy, 2011, 19(6): 1070-1078; U.S. Pat. Nos. 6,156,303; 7,198,951; U.S. Patent Publication Nos.: US2015/0159173 and US2014/0359799; and International Patent Publication NOs.: WO1998/011244, WO2005/033321 and WO2014/14422.

AAV vectors include not only single stranded vectors but self-complementary AAV vectors (scAAVs). scAAV vectors contain DNA which anneals together to form double stranded vector genome. By skipping second strand synthesis, scAAVs allow for rapid expression in the cell. The rAAV vectors may be manufactured by standard methods in the art such as by triple transfection, in sf9 insect cells or in suspension cell cultures of human cells such as HEK293 cells.

In some embodiments, non-viral based methods may be used. For instance, in some aspects, vectors comprising the polynucleotides may be transferred to cells by non-viral methods by physical methods such as needles, electroporation, sonoporation, hydroporation; chemical carriers such as inorganic particles (e.g. calcium phosphate, silica, gold) and/or chemical methods. In other aspects, synthetic or natural biodegradable agents may be used for delivery such as cationic lipids, lipid nano emulsions, nanoparticles, peptide based vectors, or polymer based vectors.

In some embodiments, the polynucleotide encoding the heterologous agent(s) (e.g. CAR and/or immunomodulator, such as a cytokine) is designed as a messenger RNA (mRNA) for delivery. In some embodiments, the polynucleotide encoding the antigen receptor, such as a CAR, is designed as a messenger RNA (mRNA) for delivery. In some embodiments, the polynucleotide encoding the immunomodulator, such as a cytokine, is designed as a messenger RNA (mRNA) for delivery. In some embodiments, the polynucleotides encoding the antigen receptor, such as a CAR, and the immunomodulator, such as a cytokine, are designed as a messenger RNA (mRNA) for delivery.

In some embodiments, the polynucleotide, such as mRNA, encoding the heterologous agent(s) (e.g. CAR and/or immunomodulator, such as a cytokine) is incorporated in lipid nanoparticles. In some embodiments, the polynucleotide, such as mRNA, encoding the antigen receptor, such as a CAR, is incorporated in lipid nanoparticles. In some embodiments, the polynucleotide, such as mRNA, encoding the immunomodulator, such as a cytokine, is incorporated in lipid nanoparticles. In some embodiments, the polynucleotides, such as mRNA, encoding the antigen receptor, such as a CAR, and the immunomodulator, such as a cytokine, are incorporated in lipid nanoparticles. In some embodiments, the formulation is a nanoparticle which may comprise at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG and PEGylated lipids. In another aspect, the lipid may be a cationic lipid such as, but not limited to, DLin-DMA, DLin-D-DMA, DLin-MC 3-DMA, DLin-KC2-DMA and DODMA

Lipid nanoparticles can be used for the delivery of encapsulated or associated (e.g., complexed) therapeutic agents, including mRNA. In particular, some nanoparticle compositions are particularly useful for the delivery of nucleic acids including messenger RNA (mRNA), antisense oligonucleotide, plasmid DNA, microRNA (miRNA), miRNA inhibitors (antagomirs/antimers), messenger-RNA-interfering complementary RNA (micRNA), DNA, multivalent RNA, dicer substrate RNA, complementary DNA (cDNA), and self-amplifying RNA (saRNA). See, e.g., U.S. Pat. No. 10,723,692 B2.

Thus, among provided methods herein are methods for the delivery of nucleic acids including DNA, RNA, mRNA, and self-amplifying RNA (saRNA) encoding a heterologous agent(s) (e.g. CAR and/or immunomodulator, such as a cytokine) for delivery into g-NK cells or a composition or population of cells enriched in g-NK cells. Also provided are methods for the delivery of nucleic acids including DNA, RNA, mRNA, and self-amplifying RNA (saRNA) encoding the antigen receptor, such as a CAR, for delivery into g-NK cells or a composition or population of cells enriched in g-NK cells. Also provided are methods for the delivery of nucleic acids including DNA, RNA, mRNA, and self-amplifying RNA (saRNA) encoding the immunomodulator, such as a cytokine, for delivery into g-NK cells or a composition or population of cells enriched in g-NK cells. Also provided are methods for the delivery of nucleic acids including DNA, RNA, mRNA, and self-amplifying RNA (saRNA) encoding the antigen receptor, such as a CAR, and the immunomodulator, such as a cytokine, for delivery into g-NK cells or a composition or population of cells enriched in g-NK cells. In some embodiments, the heterologous agent(s) (e.g. CAR and/or immunomodulator, such as a cytokine) are packaged or incorporated into lipid nanoparticles for delivery of the nucleic acid, e.g. DNA, RNA, mRNA, and self-amplifying RNA (saRNA). In some embodiments, the antigen receptor, such as a CAR, is packaged or incorporated into lipid nanoparticles for delivery of the nucleic acid, e.g. DNA, RNA, mRNA, and self-amplifying RNA (saRNA). In some embodiments, the immunomodulator, such as a cytokine, is packaged or incorporated into lipid nanoparticles for delivery of the nucleic acid, e.g. DNA, RNA, mRNA, and self-amplifying RNA (saRNA). In some embodiments, the antigen receptor, such as a CAR, and the immunomodulator, such as a cytokine, are packaged or incorporated into lipid nanoparticles for delivery of the nucleic acid, e.g. DNA, RNA, mRNA, and self-amplifying RNA (saRNA). In some embodiments, the nucleic acid is DNA. In some embodiments, the nucleic acid is RNA. In some embodiments, the nucleic acid is mRNA. In some embodiments, the nucleic acid is self-amplifying RNA (saRNA).

In some embodiments, the mRNA is a self-amplifying mRNA. Self-amplifying RNA (saRNA) is able to self-amplify itself through the presence of 5′ and 3′ conserved sequence elements (CSEs) and nsP1-4 genes along with a subgenomic promoter. See, e.g., Bloom, van den Berg, and Arbuthnot, Gene Therapy, 2021. Following in situ translation, the nsP1-4 proteins form an RdRP complex which recognizes the flaking CSE sequences and amplifies the sequence contained within the RNA. Introduction of saRNA to a target cell can be performed via lipid nanoparticle delivery. In some embodiments, such self-amplifying RNA may have structural features or components of any of those taught in International Patent Application Publication No. WO201 1 05799.

In some embodiments, the provided methods involve use of a lipid nanoparticle (LNP) comprising mRNA encoding a heterologous agent(s) (e.g. CAR and/or immunomodulator, such as a cytokine). In some embodiments, the provided methods involve use of a lipid nanoparticle (LNP) comprising mRNA encoding the antigen receptor, such as a CAR. In some embodiments, the provided methods involve use of a lipid nanoparticle (LNP) comprising mRNA encoding the immunomodulator, such as a cytokine. In some embodiments, the provided methods involve use of a lipid nanoparticle (LNP) comprising mRNA encoding the antigen receptor, such as a CAR, and the immunomodulator, such as a cytokine. In some embodiments, the mRNA encoding a heterologous agent(s) (e.g. CAR and/or immunomodulator, such as a cytokine) can be produced using methods known in the art such as in vitro transcription. In some embodiments, the mRNA encoding the antigen receptor, such as a CAR, can be produced using methods known in the art such as in vitro transcription. In some embodiments, the mRNA encoding the immunomodulator, such as a cytokine, can be produced using methods known in the art such as in vitro transcription. In some embodiments, the mRNA encoding the antigen receptor, such as a CAR, and the immunomodulator, such as a cytokine, can be produced using methods known in the art such as in vitro transcription. In some embodiments of the method, the mRNA comprises a 5′ cap. In some embodiments, the 5′ cap is an altered nucleotide on the 5′ end of primary transcripts such as messenger RNA. In some aspects, the 5′ caps of the mRNA improves one or more of RNA stability and processing, mRNA metabolism, the processing and maturation of an RNA transcript in the nucleus, transport of mRNA from the nucleus to the cytoplasm, mRNA stability, and efficient translation of mRNA to protein. In some embodiments, a 5′ cap can be a naturally-occurring 5′ cap or one that differs from a naturally-occurring cap of an mRNA. A 5′ caps may be any 5′ caps known to a skilled artisan. In certain embodiments, the 5′ cap is selected from the group consisting of an Anti-Reverse Cap Analog (ARCA) cap, a 7-methyl-guanosine (7mG) cap, a CleanCap® analog, a vaccinia cap, and analogs thereof. For instance, the 5′ cap may include, without limitation, an anti-reverse cap analogs (ARCA) (U.S. Pat. No. 7,074,596), 7-methyl-guanosine, CleanCap® analogs, such as Cap 1 analogs (Trilink; San Diego, CA), or enzymatically capped using, for example, a vaccinia capping enzyme or the like. In some embodiments, the mRNA may be polyadenylated. The mRNA may contain various 5′ and 3′ untranslated sequence elements to enhance expression of the encoded engineered heterologous agent(s) (e.g. CAR and/or immunomodulator, such as a cytokine) and/or stability of the mRNA itself. The mRNA may contain various 5′ and 3′ untranslated sequence elements to enhance expression of the encoded engineered antigen receptor, such as a CAR, and/or stability of the mRNA itself. The mRNA may contain various 5′ and 3′ untranslated sequence elements to enhance expression of the encoded engineered immunomodulator, such as a cytokine, and/or stability of the mRNA itself. The mRNA may contain various 5′ and 3′ untranslated sequence elements to enhance expression of the encoded engineered antigen receptor, such as a CAR, and the immunomodulator, such as a cytokine, and/or stability of the mRNA itself. Such elements can include, for example, posttranslational regulatory elements such as a woodchuck hepatitis vims posttranslational regulatory element.

In some embodiments, the mRNA comprises at least one nucleoside modification. The mRNA may contain modifications of naturally-occurring nucleosides to nucleoside analogs. Any nucleoside analogs known in the art are envisioned. Such nucleoside analogs can include, for example, those described in U.S. Pat. No. 8,278,036. In certain embodiments of the method, the nucleoside modification is selected from the group consisting of a modification from uridine to pseudouridine and uridine to Nl-methyl pseudouridine. In particular embodiments of the method the nucleoside modification is from uridine to pseudouridine.

LNPs particularly useful for in the present methods comprise a cationic lipid selected from DLin-DMA (1,2-dilinoleyloxy-3-dimethylaminopropane), DLin-MC3-DM A (dilinoleylmethyl-4-dimethylaminobutyrate), DLin-KC2-DMA (2,2-dilinoleyl-4-(2-dimethylaminoethyl)-[1,3]-dioxolane), DODMA (1,2-dioleyloxy-N,N-dimethyl-3-aminopropane), SS-OP (Bis[2-(4-{2-[4-(cis-9 octadecenoyloxy)phenylacetoxy]ethyl}piperidinyl)ethyl] disulfide), and derivatives thereof. DLin-MC3-DMA and derivatives thereof are described, for example, in WO 2010144740. DODMA and derivatives thereof are described, for example, in U.S. Pat. No. 7,745,651 and Mok et al. (1999), Biochimica et Biophysica Acta, 1419(2): 137-150. DLin-DMA and derivatives thereof are described, for example, in U.S. Pat. No. 7,799,565. DLin-KC2-DMA and derivatives thereof are described, for example, in U.S. Pat. No. 9,139,554. SS-OP (NOF America Corporation, White Plains, NY) is described, for example, at www. nofamerica.com/store/index.php?dispatch=products. view &product_id=962. Additional and non-limiting examples of cationic lipids include methylpyridiyl-dialkyl acid (MPDACA), palmitoyl-oleoyl-nor-arginine (PONA), guanidino-dialkyl acid (GUADACA), 1,2-di-0-octadecenyl-3-trimethylammonium propane (DOTMA), 1,2-dioleoyl-3-trimethylammonium-propane (DOTAP), Bis{2-[N-methyl-N-(a-D-tocopherolhemisuccinatepropyl)amino]ethyl} disulfide (SS-33/3AP05), Bis{2-[4-(a-D-tocopherolhemisuccinateethyl)piperidyl] ethyl} disulfide (SS33/4PE15), Bis{2-[4-(cis-9-octadecenoateethyl)-1-piperidinyl] ethyl} disulfide (SS18/4PE16), and Bis{2-[4-(cis,cis-9,12-octadecadienoateethyl)-1-piperidinyl] ethyl} disulfide (SS18/4PE13). In further embodiments, the lipid nanoparticles also comprise one or more non-cationic lipids and a lipid conjugate.

In some embodiments, the molar concentration of the cationic lipid is from about 20% to about 80%, from about 30% to about 70%, from about 40% to about 60%, from about 45% to about 55%, or about 20%, about 25%, about 30%, about 35%, about 40%, about 45%, about 50%, about 55%, about 60%, about 65%, about 70%, about 75%, or about 80% of the total lipid molar concentration, wherein the total lipid molar concentration is the sum of the cationic lipid, the non-cationic lipid, and the lipid conjugate molar concentrations. In certain embodiments, the lipid nanoparticles comprise a molar ratio of cationic lipid to mRNA of from about 1 to about 20, from about 2 to about 16, from about 4 to about 12, from about 6 to about 10, or about 1, about 2, about 3, about 4, about 5, about 6, about 7, about 8, about 9, about 10, about 11, about 12, about 13, about 14, about 15, about 16, about 17, about 18, about 19, or about 20.

In some embodiments, the lipid nanoparticles utilized in the presently disclosed methods can comprise at least one non-cationic lipid. In particular embodiments, the molar concentration of the non-cationic lipids is from about 20% to about 80%, from about 30% to about 70%, from about 40% to about 70%, from about 40% to about 60%, from about 46% to about 50%, or about 20%, about 25%, about 30%, about 35%, about 40%, about 45%, about 48.5%, about 50%, about 55%, about 60%, about 65%, about 70%, about 75%, or about 80% of the total lipid molar concentration. Non-cationic lipids include, in some embodiments, phospholipids and steroids.

In some embodiments, phospholipids useful for the lipid nanoparticles described herein include, but are not limited to, 1,2-Distearoyl-sn-glycero-3-phosphocholine (DSPC), 1,2-Didecanoyl-sn-glycero-3-phosphocholine (DDPC), 1,2-Dierucoyl-sn-glycero-3-phosphate (Sodium Salt) (DEPA-NA), 1,2-Dierucoyl-sn-glycero-3-phosphocholine (DEPC), 1,2-Dierucoyl-sn-glycero-3-phosphoethanolamine (DEPE), 1,2-Dierucoyl-sn-glycero-3[Phospho-rac-(1-glycerol) (Sodium Salt) (DEPG-NA), 1,2-Dilinoleoyl-sn-glycero-3-phosphocholine (DLOPC), 1,2-Dilauroyl-sn-glycero-3-phosphate (Sodium Salt) (DLPA-NA), 1,2-Dilauroyl-sn-glycero-3-phosphocholine (DLPC), 1,2-Dilauroyl-sn-glycero-3-phosphoethanolamine (DLPE), 1,2-Dilauroyl-sn-glycero-3[Phospho-rac-(1-glycerol . . . ) (Sodium Salt) (DLPG-NA), 1,2-Dilauroyl-sn-glycero-3[Phospho-rac-(1-glycerol)(Ammonium Salt) (DLPG-NH4), 1,2-Dilauroyl-sn-glycero-3-phosphoserine (Sodium Salt) (DLPS-NA), 1,2-Dimyristoyl-sn-glycero-3-phosphate (SodiumSalt) (DMPA-NA), 1,2-Dimyristoyl-sn-glycero-3-phosphocholine (DMPC), 1,2-Dimyristoyl-sn-glycero-3-phosphoethanolamine (DMPE), 1,2-Dimyristoyl-sn-glycero-3[Phospho-rac-(1-glycerol) (Sodium Salt) (DMPG-NA), 1,2-Dimyristoyl-sn-glycero-3[Phospho-rac-(1-glycerol)(Ammonium Salt) (DMPG-NH4), 1,2-Dimyristoyl-sn-glycero-3[Phospho-rac-(1-glycerol)(Sodium/Ammonium Salt) (DMPG-NH4/NA), 1,2-Dimyristoyl-sn-glycero-3-phosphoserine (Sodium Salt) (DMPS-NA), 1,2-Dioleoyl-sn-glycero-3-phosphate (Sodium Salt) (DOPA-NA), 1,2-Dioleoyl-sn-glycero-3-phosphocholine (DOPC), 1,2-Dioleoyl-sn-glycero-3-phosphoethanolamine (DOPE), 1,2-Dioleoyl-sn-glycero-3[Phospho-rac-(1-glycerol) (Sodium Salt) (DOPG-NA), 1,2-Dioleoyl-sn-glycero-3-phosphoserine (Sodium Salt) (DOPS-NA), 1,2-Dipalmitoyl-sn-glycero-3-phosphate (Sodium Salt) (DPPA-NA), 1,2-Dipalmitoyl-sn-glycero-3-phosphocholine (DPPC), 1,2-Dipalmitoyl-sn-glycero-3-phosphoethanolamine (DPPE), 1,2-Dipalmitoyl-sn-glycero-3[Phospho-rac-(1-glycerol) (Sodium Salt) (DPPG-NA), 1,2-Dipalmitoyl-sn-glycero-3[Phospho-rac-(1-glycerol)(Ammonium Salt) (DPPG-NH4), 1,2-Dipalmitoyl-sn-glycero-3-phosphoserine (Sodium Salt) (DPPS-NA), 1,2-Distearoyl-sn-glycero-3-phosphate (Sodium Salt) (DSPA-NA), 1,2-Distearoyl-sn-glycero-3-phosphoethanolamine (DSPE), 1,2-Distearoyl-sn-glycero-3[Phospho-rac-(1-glycerol) (Sodium Salt) (DSPG-NA), 1,2-Distearoyl-sn-glycero-3[Phospho-rac-(1-glycerol)(Ammonium Salt) (DSPG-NH4), 1,2-Distearoyl-sn-glycero-3-phosphoserine (Sodium Salt) (DSPS-NA), Egg-PC (EPC), Hydrogenated Egg PC (HEPC), Hydrogenated Soy PC (HSPC), 1-Myristoyl-sn-glycero-3-phosphocholine (LYSOPCMYRISTIC), 1-Palmitoyl-sn-glycero-3-phosphocholine (LYSOPCPALMITIC), 1-Stearoyl-sn-glycero-3-phosphocholine (LYSOPC STEARIC), 1-Myristoyl-2-palmitoyl-sn-glycero3-phosphocholine (MPPC), 1-Myristoyl-2-stearoyl-sn-glycero-3-phosphocholine (MSPC), 1-Palmitoyl-2-myristoyl-sn-glycero-3-phosphocholine (PMPC), 1-Palmitoyl-2-oleoyl-sn-glycero-3-phosphocholine (POPC), 1-Palmitoyl-2-oleoyl-sn-glycero-3-phosphoethanolamine (POPE), 1-Palmitoyl-2-oleoyl-sn-glycero-3[Phospho-rac-(1-glycerol)] (Sodium Salt) (POPG-NA), 1-Palmitoyl-2-stearoyl-sn-glycero-3-phosphocholine (PS PC), 1-Stearoyl-2-myristoyl-sn-glycero-3-phosphocholine (SMPC), 1-Stearoyl-2-oleoyl-sn-glycero-3-phosphocholine (SOPC), and 1-Stearoyl-2-palmitoyl-sn-glycero-3-phosphocholine (SPPC). In particular embodiments, the phospholipid is DSPC. In particular embodiments, the phospholipid is DOPE. In particular embodiments, the phospholipid is DOPC.

In some embodiments, the non-cationic lipids comprised by the lipid nanoparticles include one or more steroids. Steroids useful for the lipid nanoparticles described herein include, but are not limited to, cholestanes such as cholesterol, cholanes such as cholic acid, pregnanes such as progesterone, androstanes such as testosterone, and estranes such as estradiol. Further steroids include, but are not limited to, cholesterol (ovine), cholesterol sulfate, desmosterol-d6, cholesterol-d7, lathosterol-d7, desmosterol, stigmasterol, lanosterol, dehydrocholesterol, dihydrolanosterol, zymosterol, lathosterol, zymosterol-d5, 14-demethyl-lanosterol, 14-demethyl-lanosterol-d6, 8(9)-dehydrocholesterol, 8(14)-dehydrocholesterol, diosgenin, DHEA sulfate, DHEA, lanosterol-d6, dihydrolanosterol-d7, campesterol-d6, sitosterol, lanosterol-95, Dihydro FF-MAS-d6, zymostenol-d7, zymostenol, sitostanol, campestanol, campesterol, 7-dehydrodesmosterol, pregnenolone, sitosterol-d7, Dihydro T-MAS, Delta 5-avenasterol, Brassicasterol, Dihydro FF-MAS, 24-methylene cholesterol, cholic acid derivatives, cholesteryl esters, and glycosylated sterols. In particular embodiments, the lipid nanoparticles comprise cholesterol.

In some embodiments, the lipid nanoparticles comprise a lipid conjugate. Such lipid conjugates include, but are not limited to, ceramide PEG derivatives such as C8 PEG2000 ceramide, C16 PEG2000 ceramide, C8 PEG5000 ceramide, C16 PEG5000 ceramide, C8 PEG750 ceramide, and C16 PEG750 ceramide, phosphoethanolamine PEG derivatives such as 16:0 PEG5000PE, 14:0 PEG5000 PE, 18:0 PEG5000 PE, 18:1 PEG5000 PE, 16:0 PEG3000 PE, 14:0 PEG3000 PE, 18:0 PEG3000 PE, 18:1 PEG3000 PE, 16:0 PEG2000 PE, 14:0 PEG2000 PE, 18:0 PEG2000 PE, 18:1 PEG2000 PE 16:0 PEG1000 PE, 14:0 PEG1000 PE, 18:0 PEG1000 PE, 18:1 PEG 1000 PE, 16:0 PEG750 PE, 14:0 PEG750 PE, 18:0 PEG750 PE, 18:1 PEG750 PE, 16:0 PEG550 PE, 14:0 PEG550 PE, 18:0 PEG550 PE, 18:1 PEG550 PE, 16:0 PEG350 PE, 14:0 PEG350 PE, 18:0 PEG350 PE, and 18:1 PEG350, sterol PEG derivatives such as Chol-PEG600, and glycerol PEG derivatives such as DMG-PEG5000, DSG-PEG5000, DPG-PEG5000, DMG-PEG3000, DSG-PEG3000, DPG-PEG3000, DMG-PEG2000, DSG-PEG2000, DPG-PEG2000, DMG-PEG1000, DSG-PEG1000, DPG-PEG1000, DMG-PEG750, DSG-PEG750, DPG-PEG750, DMG-PEG550, DSG-PEG550, DPG-PEG550, DMG-PEG350, DSG-PEG350, and DPG-PEG350. In some embodiments, the lipid conjugate is a DMG-PEG. In some particular embodiments, the lipid conjugate is DMG-PEG2000. In some particular embodiments, the lipid conjugate is DMG-PEG5000.

It is within the level of a skilled artisan to select the cationic lipids, non-cationic lipids and/or lipid conjugates which comprise the lipid nanoparticle, as well as the relative molar ratio of such lipids to each other, such as based upon the characteristics of the selected lipid(s), the nature of the delivery to the intended target cells (e.g. g-NK cell enriched composition), and the characteristics of the mRNA to be delivered. Additional considerations include, for example, the saturation of the alkyl chain, as well as the size, charge, pH, pKa, fusogenicity and toxicity of the selected lipid(s). Thus, the molar ratios of each individual component may be adjusted accordingly.

The lipid nanoparticles for use in the method can be prepared by various techniques which are known to a skilled artisan. Nucleic acid-lipid particles and their method of preparation are disclosed in, for example, U.S. Patent Publication Nos. 20040142025 and 20070042031.

In some embodiments, the lipid nanoparticles will have a size within the range of about 25 to about 500 nm. In some embodiments, the lipid nanoparticles have a size from about 50 nm to about 300 nm, or from about 60 nm to about 120 nm. The size of the lipid nanoparticles may be determined by quasi-electric light scattering (QELS) as described in Bloomfield, Ann. Rev. Biophys. Bioeng., 10:421A150 (1981). A variety of methods are known in the art for producing a population of lipid nanoparticles of particular size ranges, for example, sonication or homogenization. One such method is described in U.S. Pat. No. 4,737,323.

In some embodiments, the lipid nanoparticles comprise an immune cell targeting molecule such as, for example, a targeting ligand (e.g., antibodies, scFv proteins, DART molecules, peptides, aptamers, and the like) anchored on the surface of the lipid nanoparticle that selectively binds the lipid nanoparticles to NK cells, e.g. g-NK cells.

In some embodiments, introduction of the nucleic acid can be performed through electroporation. In some embodiments, the nucleic acid is introduced to the g-NK cell via electroporation. In some embodiments, the nucleic acid is DNA. In some embodiments, the nucleic acid is RNA. In some embodiments, the RNA is mRNA. In some embodiments, the RNA is saRNA. In some embodiments, the nucleic acid, such as an mRNA or saRNA, is incorporated into a lipid nanoparticle for delivery by electroporation.

### III. GENE EDITING

In some embodiments, the g-NK cell may be genetically engineered by gene editing to alter (e.g.) reduce expression of one or more genes by the g-NK cells, thereby altering one or more properties or activities of the NK cells. For instance, strategies for gene editing can include one or more strategy that reduces fratricide (self-killing) due to expression of target antigen on g-NK cells; reduces undesired immunoreactivity that may result in graft vs. host disease (GvHD) particularly when infused into immune-compromised HLA-matched or, in some cases also when infused into HLA mis-mismatched recipients; or reduces immunosuppression by host factors, particularly in the tumor microenvironment. In some embodiments, the engineered g-NK cells, including those engineered by one or more gene editing strategy, exhibit enhanced NK cell response characteristics as compared to similar NK cells without the gene editing, e.g., enhanced target recognition, enhanced NK cell response level and/or duration, improved NK cell survival, delayed NK cell exhaustion, and/or enhanced target recognition.

In some embodiments, the g-NK cells are generated by gene editing to disrupt or knock out the gene encoding FcRγ chain. In some embodiments the NK cell is genetically engineered to reduce or eliminate expression or activity of human FcRγ chain protein. In some embodiments, the genetic disruption results in an insertion, deletion or mutation in the gene, such as a frameshift mutations and/or premature stop codons within the open reading frame. Methods for knockout or disruption of FcRγ chain in NK cells are described in PCT publ. No. WO2018/148462 and Liu et al. iScience, 2020; 23:101709.

One of ordinary skill in the art will understand that there are many suitable methods for disrupting FcRγ chain gene. For example, the entire gene locus, such as FcRγ locus, may be deleted. In some cases, it is also suitable to delete a portion of the gene, for example an exon, or a domain. Specifically, the ITAM signaling domain of FcRγ may be deleted. Alternatively, the provided methods also include introducing one or more amino acid substitutions into the gene locus, such as FcRγ locus, such as an inactivating mutation. In some embodiments, a stop codon can be introduced into the mRNA, such as FcRγ mRNA, to produce a truncated and/or inactivated form of the expressed gene, such as FcRγ signaling adaptor. In some embodiments, regulatory elements of the gene, such as FcRγ gene, can also be mutated or deleted in order to reduce expression, activity and/or signaling of FcRγ signaling adaptor.

In some embodiments, gene disruption can be carried out in mammalian cells using site-specific endonucleases. Endonucleases that allow for site-specific deletion of a gene are well known in the art and may include TAL nucleases, meganucleases, zinc-finger nucleases, CRISPR/Cas (e.g. Cas9), and Argonaute. Methods for producing engineered, site-specific endonucleases are known in the art. The site-specific endonuclease can be engineered to recognize and delete or modify a specific gene, such as the FcRγ chain gene.

In some embodiments, provided g-NK are engineered by editing the genome of the g-NK cells. In some embodiments, the editing of the genome may be carried out in a method that enriches for g-NK cell subset from a starting sample of NK cells. Thus, it is understood that the provided methods do not require selecting editing the genome only of g-NK cells that have been selected for NK cells that are deficient in the FcRγ chain (or only that have been selected or identified by a g-NK surrogate marker profile), but may involve gene editing of a composition of NK cells that are to be, or that have been, preferentially expanded or enriched in g-NK cells. As such, the final composition of cells that are enriched in g-NK cells include g-NK cells introduced with the heterologous antigen receptor (e.g. CAR), with the heterologous cytokine (e.g. secretable or membrane-bound interleukin, such as IL-15 or IL-21), or with the heterologous antigen receptor and cytokine and that have been gene edited. Exemplary methods for preparing and expanding a composition enriched in g-NK cells is provided in Section V.

In some embodiments, the editing of the genome may take place at any suitable time during the methods of expanding the g-NK cells, such as described in Section V. In some embodiments, the gene editing is carried out after the selection of cells from a subject (e.g. selecting or enriching cells that are CD3^{neg}CD57^{pos} or CD3^{neg}CD56^{pos}) and prior to incubating or culturing the selected or enriched cells with feeder cells (e.g. HLA-E-expressing feeder cells) for proliferation or expansion of the NK cells. In some embodiments, the gene editing is carried out after the incubation or culture with the feeder cells (e.g. HLA-E-expressing feeder cells) and thus after selected or enriched cells have proliferated or expanded. In some embodiments, the gene editing is carried out sequentially, in any order, with the methods for introducing an antigen receptor (e.g. CAR) and/or an immunomodulator (e.g. cytokine, such as a secretable or membrane-bound interleukin, e.g. IL-15 or IL-21). In some embodiments, the gene editing is carried out sequentially, in any order, with the methods for introducing the antigen receptor, such as a CAR. In some embodiments, the gene editing is carried out sequentially, in any order, with the methods for introducing the immunomodulator, such as a cytokine. In some embodiments, the gene editing is carried out sequentially, in any order, with the methods for introducing the antigen receptor, such as a CAR, and the immunomodulator, such as a cytokine.

Methods for knocking out a target gene expression include, but not limited to, a zinc finger nuclease (ZFN), a Tale-effector domain nuclease (TALEN), and CRIPSR/Cas system. Such methods typically comprise administering to the cell one or more polynucleotides encoding one or more nucleases such that the nuclease mediates modification of the endogenous gene, for example in the presence of one or more donor sequence, such that the donor is integrated into the endogenous gene targeted by the nuclease. Integration of one or more donor molecule(s) occurs via homology-directed repair (HDR) or by non-homologous end joining (NHEJ) associated repair. In certain embodiments, one or more pairs of nucleases are employed, which nucleases may be encoded by the same or different nucleic acids.

In some embodiments, gene editing is carried out using a zinc finger nuclease (ZFN). ZFNs are fusion proteins that comprise a non-specific cleavage domain (N) of FokI endonuclease and a zinc finger protein (ZFP). A pairs of ZNFs are involved to recognize a specific locus in a target gene—one that recognizes the sequence upstream and the other that recognizes the sequence downstream of the site to be modified—and the nuclease portion of the ZFN cuts at the specific locus and causing the knock out of the target gene. Methods of using the ZFNs to reduce gene expression is well known, for example, as disclosed in U.S. Pat. No. 9,045,763, and also in Durai et al., “Zinc Finger Nucleases: Custom-Designed Molecular Scissors for Genome Engineering of Plant and Mammalian cells,” Nucleic Acid Research 33 (18):5978-5990 (2005), the disclosures of which are incorporated by reference in its entirety.

In some embodiments, gene editing is carried out using transcription activator-like effector nucleases (TALENS). TALENs are similar to ZFNs in that they bind as a pair around a genomic site and direct the same non-specific nuclease, FoKI, to cleave the genome at a specific site, but instead of recognizing DNA triplets, each domain recognizes a single nucleotide. Methods of using the ZFNs to reduce gene expression are also well known, for example, as disclosed in U.S. Pat. No. 9,005,973, and also Christian et al. “Targeting DNA Double-Strand Breaks with TAL Effector Nucleases,” Genetics 186(2): 757-761 (2010), the disclosures of which are incorporated by reference in their entirety.

In some embodiments, gene editing is carried out using an RNA-guided nuclease. In some embodiments, the RNA-guided nuclease is a RNA-guided DNA endonuclease. In some embodiments, the RNA-guided nuclease is a CRISPR nuclease. Non-limiting examples of RNA-guided nucleases include any as described in PCT publication No. WO2020/168300 (e.g. Table 2 therein). In some embodiments, the RNA-guided nuclease is a Cas9 or Cas12 nuclease. In some embodiments, the RNA-guided nuclease is Cpfl (Cas12a). In some embodiments, Cpfl is Acidaminococcus sp. Cpfl (AsCpfl).

In some embodiments, gene editing is carried out with an RNA-guided nuclease and a guide RNA (gRNA). These two components form a complex that is capable of associating with a specific nucleic acid sequence and editing the DNA in or around that nucleic acid sequence, for instance by making one or more of a single-strand break (an SSB or nick), a double-strand break (a DSB) and/or a point mutation. In some embodiments, the gRNA includes a crRNA and, optionally, a tracrRNA. In some embodiments, the RNA-guided nuclease (e.g. Cas9 or a Cas12) and one or more gRNAs form ribonucleoprotein (RNP) complexes that associate with (i.e. target) and cleave specific loci complementary to a targeting (or spacer) sequence of the gRNA (e.g. crRNA). In some embodiments, the Cas is a Cas9 nuclease, such as from Streptococcus pyogenes. It is understood that the endonuclease used herein is not limited to the Cas9 of Streptococcus pyogenes (SpCas9) typically used for a synthetic Cas9. In one aspect, the Cas9 can come from a different bacterial source. Substitution of the Cas9 can also be used to increase the targeting specificity so less gRNA needs to be used. Thus, for example, the Cas can be derived from Staphylococcus aureus (SaCas9), Acidaminococcus sp. (AsCpf1), Clustered Regularly Interspaced Short Palindromic Repeats from Prevotella and Francisella 1 (Cpf1) derived from Lachnospiracase bacterium (LbCpf1), Neisseria meningitidis (NmCas9), Streptococcus thermophilus (StCas9), Campylobacter jejuni (CjCas9), enhanced SpCas9 (eSpCas9), SpCas9-HF1, Fokl-Fused dCas9, or an expanded Cas9 (xCas9). Additionally other Cas endonucleases can be used in place of a Cas9 system such as, for example, CasX, CasY, Casl4, Cas4, Csn2, Cas13a, Cas13b, Cas13c, Cas13d, C2c1, or C2c3 or using any other type of engineered Cas protein including prime editing.

In some embodiments, a genome editing system containing an RNA-guided nucleases (e.g. a Cas) and a gRNA is implemented, in certain embodiments, as a protein/RNA complex (a ribonucleoprotein, or RNP) that is introduced into the cell to be edited. In some embodiments, the RNP complex is introduced into the cells in an encapsulating agent, such as a lipid or polymer micro- or nanoparticle, micelle, or liposome. In certain embodiments, a genome editing system containing an RNA-guided nucleases (e.g. a Cas) and a gRNA is implemented as one or more nucleic acids encoding the RNA-guided nuclease and guide RNA components. For instance, in certain embodiments, the genome editing system is implemented as one or more vectors comprising such nucleic acids, for instance a viral vector such as an adeno-associated virus.

In functional terms, RNA-guided nucleases are defined as those nucleases that: (a) interact with (e.g., complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM.” The PAM sequence takes its name from its sequential relationship to the “protospacer” sequence that is complementary to gRNA targeting domains (or “spacers”). Together with protospacer sequences, PAM sequences define target regions or sequences for specific RNA-guided nuclease/gRNA combinations. Various RNA-guided nucleases may require different sequential relationships between PAMs and protospacers. For example, Cas9 nucleases recognize PAM sequences that are 3′ of the protospacer, while Cpf1, on the other hand, generally recognizes PAM sequences that are 5′ of the protospacer. In addition to recognizing specific sequential orientations of PAMs and protospacers, RNA-guided nucleases can also recognize specific PAM sequences. S. aureus Cas9, for instance, recognizes a PAM sequence of NNGRRT or NNGRRV, wherein the N residues are immediately 3′ of the region recognized by the gRNA targeting domain. S. pyogenes Cas9 recognizes NGG PAM sequences. And F. novicida Cpf1 recognizes a TTN PAM sequence. PAM sequences have been identified for a variety of RNA-guided nucleases, and a strategy for identifying novel PAM sequences has been described by Shmakov el al, 2015, Molecular Cell 60, 385-397, Nov. 5, 2015.

It is understood and herein contemplated that the use of a particular Cas can change the PAM sequence which the Cas endonuclease (or alternative) uses to screen for targets. As used herein, suitable PAM sequences comprises NGG (SpCas9 PAM) NNGRRT (SaCas9 PAM) NNNNGATT (NmCAs9 PAM), NNNNRYAC (CjCas9 PAM), NNAGAAW (St), TTTV (LbCpf1 PAM and AsCpf1 PAM); TYCV (LbCpf1 PAM variant and AsCpf1 PAM variant); where N can be any nucleotide; V=A, C, or G; Y=C or T; W=A or T; and R=A or G.

In some embodiments, the gRNA promotes the specific association (or “targeting”) of an RNA-guided nuclease (e.g. a Cas, such as a Cas9 or a Cpf1) to a target sequence such as a genomic sequence in a cell. gRNAs can be unimolecular (comprising a single RNA molecule, and referred to alternatively as chimeric), or modular (comprising more than one, and typically two, separate RNA molecules, such as a crispr RNA (crRNA) and a tracrRNA, which are usually associated with one another, for instance by duplexing). Guide RNAs, whether unimolecular or modular, include a “targeting domain” that is fully or partially complementary to a target domain within a target sequence, such as a DNA sequence in the genome of a cell where editing is desired. For instance, in connection with a Cas9 the crRNA is the guide RNA that provides the targeting domain that is a nucleotide sequence complementary to the target DNA, and also can include a tracr RNA that serves as a binding scaffold for the Cas nuclease. In connection with Cpf1, which induces double stranded DNA breaks under the guidance of a single crRNA, a tracrRNA is not required and instead the crRNA includes a 5′-handle engaging Cpf1 recognition and a guide segment interacting with targeted DNA sequences through complementary binding. Targeting domains are typically 10-30 nucleotides in length, and in certain embodiments are 16-24 nucleotides in length (for instance, 16, 17, 18, 19, 20, 21, 22, 23 or 24 nucleotides in length).

In some embodiments, the gRNA, in some cases the crRNA, is any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optionally aligned using a suitable alignment algorithm, is about or more than about 50%, 60%, 75%, 80%, 85%, 90%, 95%, 97.5%, 99% or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g. the Burrows Wheeler Aligner), Clustal 1W, Clustal X, BLAT, and others known to a skilled artisan. The ability of a guide sequence (within a nucleic-acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid targeting complex, followed by an assessment of preferential targeting (e.g., cleavage) within the target nucleic acid sequence. Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions.

Methods for designing gRNAs are known to a skilled artisan (see e.g. Cui et al. (2018) Interdisciplinary Sciences: Computational Life Sciences, 10:455-465); PCT publication No. WO2019/010384). Methods for selection and validation of target sequences as well as off-target analyses have been described previously, e.g., in Mali; Hsu; Fu et al, 2014 Nat biotechnol 32(3): 279-84, Heigwer et al, 2014 Nat methods 11(2): 122-3; Bae et al. (2014) Bioinformatics 30(10): 1473-5; and Xiao A et al. (2014) Bioinformatics 30(8): 1180-1182. As a non-limiting example, gRNA design may involve the use of a software tool to optimize the choice of potential target sequences corresponding to a user's target sequence, e.g., to minimize total off-target activity across the genome. While off-target activity is not limited to cleavage, the cleavage efficiency at each off-target sequence can be predicted, e.g., using an experimentally-derived weighting scheme.

For example, a guide RNA comprising a targeting sequence of RNA nucleotides would include the RNA sequence corresponding to the targeting domain sequence provided as a DNA sequence, and this contains uracil instead of thymidine nucleotides. For example, a guide RNA comprising a targeting domain sequence of RNA nucleotides, and described by a DNA sequence that includes thymidine molecules would have a targeting domain of the corresponding RNA sequence that is the same but including uracil instead of thymidine. As will be apparent to the skilled artisan, such a targeting sequence would be linked to a suitable guide RNA scaffold, e.g., a crRNA scaffold sequence or a chimeric crRNA/tracerRNA scaffold sequence. Suitable gRNA scaffold sequences are known to those of ordinary skill in the art. For Cpf1, for example, a suitable scaffold sequence comprises the sequence U A AUUU CU ACUCUU GU AG AU (SEQ ID NO: 16), added to the 5′-terminus of the targeting domain.

In some embodiments, efforts to enhance the clinical ADCC response to antibodies, including MM antibodies, have been challenging because NK-cells also express certain antigens that are the same as the tumor targets. These antigens include, for example, CD38 and SLAMF7. Thus, when an NK cell therapy is combined with an antibody against the target antigen (e.g. daratumumab and elotuzumab for targeting CD38 and SLAMF7, respectively), or when the NK cells express a CAR as provided herein against the target antigen, the therapy may not only target the cancer, but can also deplete the patient's NK cell population. For instance, high CD38 expression particularly results in rapid depletion of NK cells early in the daratumumab treatment course, largely eliminating this source of innate immune cells which could potentially drive even more complete tumor eradication.

In some embodiments, the NK cells are edited to reduce expression of a target antigen that is known or suspected of also being expressed at some level by the NK cells. In some embodiments, gene editing is carried out with a gRNA that targets the target antigen known or suspected of being expressed at some level by the NK cells. In some embodiments, the NK cells express a CAR directed against CD38 and CD38 expression is reduced or eliminated in the NK cells. In some embodiments, the gRNA for use in the disclosure is a gRNA targeting CD38 (see e.g. WO2019/222503, WO2021/087466 and WO2021/113853 for exemplary gRNA targeting CD38).

In some embodiments, the gRNA targets a molecule involved in immunoreactivity of the NK cell. In some embodiments, HLA class I expression on the surface of the engineered g-NK cell is reduced. The human leukocyte antigen (HLA) system is a gene complex encoding the major histocompatibility complex (MHC) proteins in humans. The HLA class I proteins all have a long alpha chain and a short beta chain, B2M. Little HLA class I can be expressed in the absence of B2M and the expression of B2M is required for HLA class I proteins to present peptides from inside the cell. The present disclosure provides g-NK cells engineered to reduce expression of B3M. Thus, these cells avoid the immune surveillance and attach by cytotoxic T cells. In some embodiments, the gRNA for use in the disclosure is a gRNA targeting beta 2 microglobulin (B2M) (see e.g. WO2020/168300, WO2018/064694, WO2015/161276, or WO2017/152015) for exemplary gRNA targeting B2M).

In some embodiments, the gRNA targets a molecule involved in immunosuppression of the NK cell activity. Suitably, engineered NK cells comprise reduced or absent checkpoint inhibitory receptor function. Suitably, the checkpoint inhibitory receptors with reduced or absent function comprise one or more or all of CD96 (TACTILE), CD 152 (CTLA4), CD223 (LAG-3), CD279 (PD-1), CD328 (SIGLEC7), SIGLEC9, TIGIT, and/or TIM-3. Suitably, the NK cell cells comprise reduced or absent checkpoint inhibitory receptor function for two or more checkpoint inhibitory receptors. Suitably, the two or more checkpoint inhibitory receptors comprise CD96 (TACTILE), CD 152 (CTLA4), or CD328 (SIGLEC7) or CD279 (PD-1).

In some embodiments the gRNA for use in the disclosure is a gRNA targeting TIGIT (see e.g. WO2020/168300 for exemplary gRNA targeting TIGIT). In some embodiments, the gRNA for use in the disclosure is a gRNA targeting PD-1 (see e.g. WO2015/161276, or WO2017/152015) for exemplary gRNA targeting PD-1).

In some embodiments the gRNA for use in the disclosure is a gRNA targeting an adenosine receptor, such as adenosine A2a receptor (ADORA2a) (see e.g. WO2020/168300 for exemplary gRNA targeting ADORA2a). In some embodiments, the gRNA for use in the disclosure is a gRNA targeting a TGF beta receptor, such as TGFbetaR2 (see e.g. WO2020/168300 for exemplary gRNA targeting TGFbetaR2). In some embodiments, the gRNA for use in the disclosure is a gRNA targeting the gene encoding cytokine-inducible SH2-containing protein (CISH) (see e.g. WO2020/168300 for exemplary gRNA targeting CISH).

In some embodiments, RNA-guided nuclease-encoding and/or gRNA encoding DNA, can be delivered by, e.g., vectors (e.g., viral or non-viral vectors), non-vector based methods (e.g., using naked DNA or DNA complexes), or a combination thereof. In some embodiments the nucleic acid encoding the RNA-guided nuclease (e.g. a Cas) and/or gRNA is delivered by AAV. Nucleic acids for gene editing can be delivered directly to cells as naked DNA or RNA, for instance by means of transfection or electroporation, or can be conjugated to molecules (e.g., N-acetylgalactosamine) promoting uptake by the target cells.

In some embodiments the RNA-guided nuclease and gRNA are delivered into cells as a ribonucleoprotein (RNP) complex. In some embodiments, the Cas and gRNA are separately purified and then assembled to form the RNP. In some embodiments, one or more RNP complexes are delivered to the cell sequentially in any order, or simultaneously. In some embodiments the RNP complex is delivered into cells by electroporation. In some embodiments the RNP complex is delivered into cells using lipid nanoparticles.

In one non-limiting example, to make the RNP complex, crRNA and tracrRNA can be mixed at a 1:1, 2:1, or 1:2 ratio of concentrations between about 50 μM and about 500 μM (for example, 50, 60, 70, 80, 90, 100, 125, 150, 175, 200, 225, 250, 275, 300, 325, 35, 375, 400, 425, 450, 475, or 500 μM), preferably between 100 μM and about 300 μM, most preferably about 200 μM at 95 C for about 5 min to form a crRNA:tracrRNA complex (i.e., the guide RNA). The crRNA:tracrRNA complex can then be mixed with between about 20 μM and about 50 μM (for example 21, 22, 23, 24, 25, 26, 27, 28, 29, 30, 31, 32, 33, 34, 35, 36, 37, 38, 39, 40, 41, 42, 43, 44, 45, 46, 47 48, 49, or 50 μM) final dilution of a Cas endonuclease (such as, for example, Cas9).

In particular embodiments, introduction of an RNP complex into NK cells, such as expanded NK cells enriched for g-NK cells as described in Section V, is by electroporation. Electroporation is a technique in which an electric field is applied to cells to increase the permeability of the cell membrane. The application of the electric filed cause a charge gradient across the membrane which draws the charged molecules such as, nucleic acid, across the cell membrane. Thus, in one aspect, disclosed herein are methods of genetically modifying an NK cell comprising obtaining guide RNA (gRNA) specific for a target DNA sequence in the NK cell; and b) introducing via electroporation into a target NK cell, a ribonucleoprotein (RNP) complex comprising a Cas endonuclease (e.g. Cas9) complexed with a corresponding CRISPR/Cas guide RNA that hybridizes to the target sequence within the genomic DNA of the NK cell.

In some embodiments, following the introduction (e.g., electroporation) of the NK cell, the now modified NK cell can be propagated in a media comprising HLA-expressing feeder cells, generally irradiated feeder cells, and cytokines (e.g. IL-2 and IL-21) as described in Section V, such as under conditions to induce stimulation, proliferation or expansion of the NK cells enriched in g-NK cells. Thus, the genetically engineered cells retain viability and proliferative potential, as they are able to be expanded post-electroporation using irradiated feeder cells. It is understood and herein contemplated that the period of culturing can be between 1 and 14 days post introduction of the RNP complex, such as post-electroporation (i.e., 1, 2, 3, 4, 5, 6, 7, 8, 9, 10, 11, 12, 13, or 14 days), such as between 3 and 7 days, for example between 4 and 6 days. In some aspect, the media for culturing the engineered NK cells can further comprise cytokines such as, for example, IL-2, IL-12, IL-15, IL-18, and/or IL-21, such as described in Section V. In some embodiments, the media contains IL-2 and IL-21.

### IV. COMPOSITIONS AND PHARMACEUTICAL FORMULATIONS

Provided herein are compositions comprising the engineered g-NK cells. In some embodiments, the engineered g-NK cells of the composition express a CAR. The composition may be comprised of a plurality of g-NK cells expressing a CAR.

In some embodiments, the engineered g-NK cells of the composition express an immunomodulator (e.g. cytokine), which may be secreted or membrane-bound. The composition may be comprised of a plurality of g-NK cells expressing an immunomodulator.

In some embodiments, the engineered g-NK cells of the composition express a CAR and an immunomodulator (e.g. cytokine), which may be secreted or membrane-bound. The composition may be comprised of a plurality of g-NK cells expressing both a CAR and an immunomodulator. In some embodiments, the engineered NK cells comprise a plurality of engineered g-NK cells. In some embodiments, greater than at or about 50% of the engineered NK cells are g-NK cells. In some embodiments, greater than at or about 60% of the engineered NK cells are g-NK cells. In some embodiments, greater than at or about 70% of the engineered NK cells are g-NK cells. In some embodiments, greater than at or about 80% of the engineered NK cells are g-NK cells. In some embodiments, greater than at or about 90% of the engineered NK cells are g-NK cells. In some embodiments, greater than at or about 95% of the engineered NK cells are g-NK cells.

In some embodiments, the composition comprises greater than at or about 50% g-NK cells. In some embodiments, the composition comprises greater than at or about 60% g-NK cells. In some embodiments, the composition comprises greater than at or about 70% g-NK cells. In some embodiments, the composition comprises greater than at or about 80% g-NK cells. In some embodiments, the composition comprises greater than at or about 90% g-NK cells. In some embodiments, the composition comprises greater than at or about 95% g-NK cells.

In some embodiments, the plurality of NK cells of the composition comprises greater than at or about 50% g-NK cells. In some embodiments, the plurality of NK cells of the composition comprises greater than at or about 60% g-NK cells. In some embodiments, the plurality of NK cells of the composition comprises greater than at or about 70% g-NK cells. In some embodiments, the plurality of NK cells of the composition comprises greater than at or about 80% g-NK cells. In some embodiments, the plurality of NK cells of the composition comprises greater than at or about 90% g-NK cells. In some embodiments, the plurality of NK cells of the composition comprises greater than at or about 95% g-NK cells.

In some embodiments, greater than at or about 20% of total cells in the composition comprise a heterologous nucleic acid encoding a CAR. In some embodiments, greater than at or about 30% of total cells in the composition comprise a heterologous nucleic acid encoding a CAR. In some embodiments, greater than at or about 40% of total cells in the composition comprise a heterologous nucleic acid encoding a CAR. In some embodiments, greater than at or about 50% of total cells in the composition comprise a heterologous nucleic acid encoding a CAR. In some embodiments, greater than at or about 60% of total cells in the composition comprise a heterologous nucleic acid encoding a CAR. In some embodiments, greater than at or about 70% of total cells in the composition comprise a heterologous nucleic acid encoding a CAR. In some embodiments, greater than at or about 80% of total cells in the composition comprise a heterologous nucleic acid encoding a CAR. In some embodiments, greater than at or about 90% of total cells in the composition comprise a heterologous nucleic acid encoding a CAR. In some embodiments, greater than at or about 95% of total cells in the composition comprise a heterologous nucleic acid encoding a CAR

In some embodiments, greater than at or about 20% of g-NK cells in the composition comprise a heterologous nucleic acid encoding a CAR. In some embodiments, greater than at or about 30% of g-NK cells in the composition comprise a heterologous nucleic acid encoding a CAR. In some embodiments, greater than at or about 40% of g-NK cells in the composition comprise a heterologous nucleic acid encoding a CAR. In some embodiments, greater than at or about 50% of g-NK cells in the composition comprise a heterologous nucleic acid encoding a CAR. In some embodiments, greater than at or about 60% of g-NK cells in the composition comprise a heterologous nucleic acid encoding a CAR. In some embodiments, greater than at or about 70% of g-NK cells in the composition comprise a heterologous nucleic acid encoding a CAR. In some embodiments, greater than at or about 80% of g-NK cells in the composition comprise a heterologous nucleic acid encoding a CAR. In some embodiments, greater than at or about 90% of g-NK cells in the composition comprise a heterologous nucleic acid encoding a CAR. In some embodiments, greater than at or about 95% of g-NK cells in the composition comprise a heterologous nucleic acid encoding a CAR

In some embodiments, greater than at or about 20% of total cells in the composition comprise a heterologous nucleic acid encoding an immunomodulator (e.g. cytokine, either secretable or membrane-bound as described). In some embodiments, greater than at or about 30% of total cells in the composition comprise a heterologous nucleic acid encoding an immunomodulator (e.g. cytokine, either secretable or membrane-bound as described). In some embodiments, greater than at or about 40% of total cells in the composition comprise a heterologous nucleic acid encoding an immunomodulator (e.g. cytokine, either secretable or membrane-bound as described). In some embodiments, greater than at or about 50% of total cells in the composition comprise a heterologous nucleic acid encoding an immunomodulator (e.g. cytokine, either secretable or membrane-bound as described). In some embodiments, greater than at or about 60% of total cells in the composition comprise a heterologous nucleic acid encoding an immunomodulator (e.g. cytokine, either secretable or membrane-bound as described). In some embodiments, greater than at or about 70% of total cells in the composition comprise a heterologous nucleic acid encoding an immunomodulator (e.g. cytokine, either secretable or membrane-bound as described). In some embodiments, greater than at or about 80% of total cells in the composition comprise a heterologous nucleic acid encoding an immunomodulator (e.g. cytokine, either secretable or membrane-bound as described). In some embodiments, greater than at or about 90% of total cells in the composition comprise a heterologous nucleic acid encoding an immunomodulator (e.g. cytokine, either secretable or membrane-bound as described). In some embodiments, greater than at or about 95% of total cells in the composition comprise a heterologous nucleic acid encoding an immunomodulator (e.g. cytokine, either secretable or membrane-bound as described).

In some embodiments, greater than at or about 20% of g-NK cells in the composition comprise a heterologous nucleic acid encoding an immunomodulator (e.g. cytokine, either secretable or membrane-bound as described). In some embodiments, greater than at or about 30% of g-NK cells in the composition comprise a heterologous nucleic acid encoding an immunomodulator (e.g. cytokine, either secretable or membrane-bound as described). In some embodiments, greater than at or about 40% of g-NK cells in the composition comprise a heterologous nucleic acid encoding an immunomodulator (e.g. cytokine, either secretable or membrane-bound as described). In some embodiments, greater than at or about 50% of g-NK cells in the composition comprise a heterologous nucleic acid encoding an immunomodulator (e.g. cytokine, either secretable or membrane-bound as described). In some embodiments, greater than at or about 60% of g-NK cells in the composition comprise a heterologous nucleic acid encoding an immunomodulator (e.g. cytokine, either secretable or membrane-bound as described). In some embodiments, greater than at or about 70% of g-NK cells in the composition comprise a heterologous nucleic acid encoding an immunomodulator (e.g. cytokine, either secretable or membrane-bound as described). In some embodiments, greater than at or about 80% of g-NK cells in the composition comprise a heterologous nucleic acid encoding an immunomodulator (e.g. cytokine, either secretable or membrane-bound as described). In some embodiments, greater than at or about 90% of g-NK cells in the composition comprise a heterologous nucleic acid encoding an immunomodulator (e.g. cytokine, either secretable or membrane-bound as described). In some embodiments, greater than at or about 95% of g-NK cells in the composition comprise a heterologous nucleic acid encoding an immunomodulator (e.g. cytokine, either secretable or membrane-bound as described).

In some embodiments, greater than at or about 20% of total cells in the composition comprise a heterologous nucleic acid(s) encoding a CAR and an immunomodulator (e.g. cytokine, either secretable or membrane-bound as described). In some embodiments, greater than at or about 30% of total cells in the composition comprise a heterologous nucleic acid(s) encoding a CAR and an immunomodulator (e.g. cytokine, either secretable or membrane-bound as described). In some embodiments, greater than at or about 40% of total cells in the composition comprise a heterologous nucleic acid(s) encoding a CAR and an immunomodulator (e.g. cytokine, either secretable or membrane-bound as described). In some embodiments, greater than at or about 50% of total cells in the composition comprise a heterologous nucleic acid(s) encoding a CAR and an immunomodulator (e.g. cytokine, either secretable or membrane-bound as described). In some embodiments, greater than at or about 60% of total cells in the composition comprise a heterologous nucleic acid(s) encoding a CAR and an immunomodulator (e.g. cytokine, either secretable or membrane-bound as described). In some embodiments, greater than at or about 70% of total cells in the composition comprise a heterologous nucleic acid(s) encoding a CAR and an immunomodulator (e.g. cytokine, either secretable or membrane-bound as described). In some embodiments, greater than at or about 80% of total cells in the composition comprise a heterologous nucleic acid(s) encoding a CAR and an immunomodulator (e.g. cytokine, either secretable or membrane-bound as described). In some embodiments, greater than at or about 90% of total cells in the composition comprise a heterologous nucleic acid(s) encoding a CAR and an immunomodulator (e.g. cytokine, either secretable or membrane-bound as described). In some embodiments, greater than at or about 95% of total cells in the composition comprise a heterologous nucleic acid(s) encoding a CAR and an immunomodulator (e.g. cytokine, either secretable or membrane-bound as described).

In some embodiments, greater than at or about 20% of g-NK cells in the composition comprise a heterologous nucleic acid(s) encoding a CAR and an immunomodulator (e.g. cytokine, either secretable or membrane-bound as described). In some embodiments, greater than at or about 30% of g-NK cells in the composition comprise a heterologous nucleic acid(s) encoding a CAR and an immunomodulator (e.g. cytokine, either secretable or membrane-bound as described). In some embodiments, greater than at or about 40% of g-NK cells in the composition comprise a heterologous nucleic acid(s) encoding a CAR and an immunomodulator (e.g. cytokine, either secretable or membrane-bound as described). In some embodiments, greater than at or about 50% of g-NK cells in the composition comprise a heterologous nucleic acid(s) encoding a CAR and an immunomodulator (e.g. cytokine, either secretable or membrane-bound as described). In some embodiments, greater than at or about 60% of g-NK cells in the composition comprise a heterologous nucleic acid(s) encoding a CAR and an immunomodulator (e.g. cytokine, either secretable or membrane-bound as described). In some embodiments, greater than at or about 70% of g-NK cells in the composition comprise a heterologous nucleic acid(s) encoding a CAR and an immunomodulator (e.g. cytokine, either secretable or membrane-bound as described). In some embodiments, greater than at or about 80% of g-NK cells in the composition comprise a heterologous nucleic acid(s) encoding a CAR and an immunomodulator (e.g. cytokine, either secretable or membrane-bound as described). In some embodiments, greater than at or about 90% of g-NK cells in the composition comprise a heterologous nucleic acid(s) encoding a CAR and an immunomodulator (e.g. cytokine, either secretable or membrane-bound as described). In some embodiments, greater than at or about 95% of g-NK cells in the composition comprise a heterologous nucleic acid(s) encoding a CAR and an immunomodulator (e.g. cytokine, either secretable or membrane-bound as described).

In particular, among the provided compositions are compositions of cells that are enriched for g-NK cells. In some embodiments, the compositions for use in the provided methods contain g-NK cells that are expanded NK cells such as produced by any of the provided methods. In some embodiments, the compositions contain NKG2C^{pos} cells or a subset thereof. In some embodiments, the compositions contain NKG2A^{neg} cells or a subset thereof. In some embodiments, the compositions contain NKG2C^{pos}/NKG2A^{neg} cells or a subset thereof.

In some embodiments, the composition comprises about 5-99% NKG2C^{pos} cells or a subset thereof, or any percentage of NKG2C^{pos} cells or a subset thereof between 5 and 99% inclusive. In some embodiments, the composition can include an increased or greater percentages of NKG2C^{pos} cells or a subset thereof relative to total NK cells or total cells compared to the percentage of NKG2C^{pos} cells or the subset thereof relative to total NK cells or total cells naturally present in the subject from which the cells were isolated. In some embodiments, the percentage is increased at least or at least about 2-fold, 3-fold, 4-fold, 5-fold, 10-fold, 20-fold, 30-fold, 40-fold, 50-fold, 60-fold, 70-fold, 80-fold, 90-fold, 100-fold, 150-fold, 200-fold or more.

In some embodiments, the composition can include at least at or about 20%, at least at or about 30%, at least at or about 40%, at least at or about 50%, at least at or about 60%, at least at or about 65%, at least at or about 70%, at least at or about 75%, at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, at least at or about 99%, or substantially 100% NKG2C^{pos} cells or a subset thereof. In some embodiments, the composition comprises more than 50% NKG2C^{pos} cells or a subset thereof. In another embodiment, the composition comprises more than 60% NKG2C^{pos} cells or a subset thereof. In another embodiment, the composition comprises more than 70% NKG2C^{pos} cells or a subset thereof. In another embodiment, the composition comprises more than 80% NKG2C^{pos} cells or a subset thereof. In some embodiments, the provided compositions include those in which the NKG2C^{pos} cells or a subset thereof make up at least at or about 60%, at least at or about 70%, at least at or about 80%, at least at or about 85%, at least at or about 90%, at least at or about 95% or more of the cells in the composition or of the NK cells in the composition. In some of any such embodiments, greater than at or about 50%, greater than at or about 60%, greater than at or about 70%, greater than at or about 80%, greater than at or about 90% or greater than at or about 95% of the NKG2C^{pos} cells in the composition comprise a heterologous nucleic acid(s) encoding a CAR. In some of any such embodiments, greater than at or about 50%, greater than at or about 60%, greater than at or about 70%, greater than at or about 80%, greater than at or about 90% or greater than at or about 95% of the NKG2C^{pos} cells in the composition comprise a heterologous nucleic acid(s) encoding an immunomodulator (e.g. cytokine, either secretable or membrane-bound as described). In some of any such embodiments, greater than at or about 50%, greater than at or about 60%, greater than at or about 70%, greater than at or about 80%, greater than at or about 90% or greater than at or about 95% of the NKG2C^{pos} cells in the composition comprise a heterologous nucleic acid(s) encoding a CAR and an immunomodulator (e.g. cytokine, either secretable or membrane-bound as described).

In some embodiments, the composition comprises about 5-99% NKG2A^{neg} cells or a subset thereof, or any percentage of NKG2A^{neg} cells or a subset thereof between 5 and 99% inclusive. In some embodiments, the composition can include an increased or greater percentages of NKG2A^{neg} cells or a subset thereof relative to total NK cells or total cells compared to the percentage of NKG2A^{neg} cells or the subset thereof relative to total NK cells or total cells naturally present in the subject from which the cells were isolated. In some embodiments, the percentage is increased at least or at least about 2-fold, 3-fold, 4-fold, 5-fold, 10-fold, 20-fold, 30-fold, 40-fold, 50-fold, 60-fold, 70-fold, 80-fold, 90-fold, 100-fold, 150-fold, 200-fold or more.

In some embodiments, the composition can include at least at or about 20%, at least at or about 30%, at least at or about 40%, at least at or about 50%, at least at or about 60%, at least at or about 65%, at least at or about 70%, at least at or about 75%, at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, at least at or about 99%, or substantially 100% NKG2A^{neg} cells or a subset thereof. In some embodiments, the composition comprises more than 50% NKG2A^{neg} cells or a subset thereof. In another embodiment, the composition comprises more than 60% NKG2A^{neg} cells or a subset thereof. In another embodiment, the composition comprises more than 70% NKG2A^{neg} cells or a subset thereof. In another embodiment, the composition comprises more than 80% NKG2A^{neg} cells or a subset thereof. In some embodiments, the provided compositions include those in which the NKG2A^{neg} cells or a subset thereof make up at least at or about 60%, at least at or about 70%, at least at or about 80%, at least at or about 85%, at least at or about 90%, at least at or about 95% or more of the cells in the composition or of the NK cells in the composition. In some of any such embodiments, greater than at or about 50%, greater than at or about 60%, greater than at or about 70%, greater than at or about 80%, greater than at or about 90% or greater than at or about 95% of the NKG2A^{neg} cells in the composition comprise a heterologous nucleic acid(s) encoding a CAR. In some of any such embodiments, greater than at or about 50%, greater than at or about 60%, greater than at or about 70%, greater than at or about 80%, greater than at or about 90% or greater than at or about 95% of the NKG2A^{neg} cells in the composition comprise a heterologous nucleic acid(s) encoding an immunomodulator (e.g. cytokine, either secretable or membrane-bound as described). In some of any such embodiments, greater than at or about 50%, greater than at or about 60%, greater than at or about 70%, greater than at or about 80%, greater than at or about 90% or greater than at or about 95% of the NKG2A^{neg} cells in the composition comprise a heterologous nucleic acid(s) encoding a CAR and an immunomodulator (e.g. cytokine, either secretable or membrane-bound as described).

In some embodiments, the composition comprises about 5-99% NKG2C^{pos}NKG2A^{neg} cells or a subset thereof, or any percentage of NKG2C^{pos}NKG2A^{neg} cells or a subset thereof between 5 and 99% inclusive. In some embodiments, the composition can include an increased or greater percentages of NKG2C^{pos}NKG2A^{neg} cells or a subset thereof relative to total NK cells or total cells compared to the percentage of NKG2C^{pos}NKG2A^{neg} cells or the subset thereof relative to total NK cells or total cells naturally present in the subject from which the cells were isolated. In some embodiments, the percentage is increased at least or at least about 2-fold, 3-fold, 4-fold, 5-fold, 10-fold, 20-fold, 30-fold, 40-fold, 50-fold, 60-fold, 70-fold, 80-fold, 90-fold, 100-fold, 150-fold, 200-fold or more.

In some embodiments, the composition can include at least at or about 20%, at least at or about 30%, at least at or about 40%, at least at or about 50%, at least at or about 60%, at least at or about 65%, at least at or about 70%, at least at or about 75%, at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, at least at or about 99%, or substantially 100% NKG2C^{pos}NKG2A^{neg} cells or a subset thereof. In some embodiments, the composition comprises more than 50% NKG2C^{pos}NKG2A^{neg} cells or a subset thereof. In another embodiment, the composition comprises more than 60% NKG2C^{pos}NKG2A^{neg} cells or a subset thereof. In another embodiment, the composition comprises more than 70% NKG2C^{pos}NKG2A^{neg} cells or a subset thereof. In another embodiment, the composition comprises more than 80% NKG2C^{pos}NKG2A^{neg} cells or a subset thereof. In some embodiments, the provided compositions include those in which the NKG2C^{pos}NKG2A^{neg} cells or a subset thereof make up at least at or about 60%, at least at or about 70%, at least at or about 80%, at least at or about 85%, at least at or about 90%, at least at or about 95% or more of the cells in the composition or of the NK cells in the composition. In some of any such embodiments, greater than at or about 50%, greater than at or about 60%, greater than at or about 70%, greater than at or about 80%, greater than at or about 90% or greater than at or about 95% of the NKG2C^{pos}NKG2A^{neg} cells in the composition comprise a heterologous nucleic acid(s) encoding a CAR. In some of any such embodiments, greater than at or about 50%, greater than at or about 60%, greater than at or about 70%, greater than at or about 80%, greater than at or about 90% or greater than at or about 95% of the NKG2C^{pos}NKG2A^{neg} cells in the composition comprise a heterologous nucleic acid(s) encoding an immunomodulator (e.g. cytokine, either secretable or membrane-bound as described). In some of any such embodiments, greater than at or about 50%, greater than at or about 60%, greater than at or about 70%, greater than at or about 80%, greater than at or about 90% or greater than at or about 95% of the NKG2C^{pos}NKG2A^{neg} cells in the composition comprise a heterologous nucleic acid(s) encoding a CAR and an immunomodulator (e.g. cytokine, either secretable or membrane-bound as described).

In some embodiments, the composition comprises about 5-99% g-NK cells, or any percentage of g-NK cells between 5 and 99% inclusive. In some embodiments, the composition can include an increased or greater percentages of g-NK cells relative to total NK cells or total cells compared to the percentage of g-NK relative to total NK cells or total cells naturally present in the subject from which the cells were isolated. In some embodiments, the percentage is increased at least or at least about 2-fold, 3-fold, 4-fold, 5-fold, 10-fold, 20-fold, 30-fold, 40-fold, 50-fold, 60-fold, 70-fold, 80-fold, 90-fold, 100-fold, 150-fold, 200-fold or more.

In some embodiments, the composition can include at least at or about 20%, at least at or about 30%, at least at or about 40%, at least at or about 50%, at least at or about 60%, at least at or about 65%, at least at or about 70%, at least at or about 75%, at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, at least at or about 99%, or substantially 100% g-NK cells. In some embodiments, the composition comprises more than 50% g-NK cells. In another embodiment, the composition comprises more than 70% g-NK cells. In another embodiment, the composition comprises more than 80% g-NK cells. In some embodiments, the provided compositions include those in which the g-NK cells make up at least at or about 60%, at least at or about 70%, at least at or about 80%, at least at or about 85%, at least at or about 90%, at least at or about 95% or more of the cells in the composition or of the NK cells in the composition. In some of any such embodiments, greater than at or about 50%, greater than at or about 60%, greater than at or about 70%, greater than at or about 80%, greater than at or about 90% or greater than at or about 95% of the g-NK cells in the composition comprise a heterologous nucleic acid(s) encoding a CAR. In some of any such embodiments, greater than at or about 50%, greater than at or about 60%, greater than at or about 70%, greater than at or about 80%, greater than at or about 90% or greater than at or about 95% of the g-NK cells in the composition comprise a heterologous nucleic acid(s) encoding an immunomodulator (e.g. cytokine, either secretable or membrane-bound as described). In some of any such embodiments, greater than at or about 50%, greater than at or about 60%, greater than at or about 70%, greater than at or about 80%, greater than at or about 90% or greater than at or about 95% of the g-NK cells in the composition comprise a heterologous nucleic acid(s) encoding a CAR and an immunomodulator (e.g. cytokine, either secretable or membrane-bound as described).

In some embodiments, the composition includes a population of a natural killer (NK) cell subset, wherein at least at or about 40%, at least at or about 50%, at least at or about 55%, at least at or about 60%, at least at or about 65%, at least at or about 70%, at least at or about 75%, at least at least at or about 80%, at least at or about 85%, at least at or about 90%, or at least at or about 95% of the cells in the composition have a g-NK cell surrogate marker profile that is CD57^{pos}. In some embodiments, from or from about 70% to at or about 90% of the cells in the composition have the phenotype CD57^{pos}. In some embodiments, at least at or about 72%, at least at or about 74%, at least at or about 76%, at least at or about 78%, at least at or about 80%, at least at or about 82%, at least at or about 84%, at least at or about 86%, at least at or about 88%, at least at or about 90%, at least at or about 92%, at least at or about 94%, at least at or about 96% or at least at or about 98% of cell in the composition have the phenotype CD57^{pos}. In some of any of the provided embodiments, at least at or about 60% of the cells in the composition comprise the phenotype CD57^{pos}. In some of any of the provided embodiments, at least at or about 70% of the cells in the composition comprise the phenotype CD57^{pos}. In some embodiments, the phenotype further includes the surface phenotype CD3^{neg}. In some embodiments, the phenotype further includes the surface phenotype CD45^{pos}/CD3^{neg}/CD56^{pos}. In some of any such embodiments, greater than at or about 50%, greater than at or about 60%, greater than at or about 70%, greater than at or about 80%, greater than at or about 90% or greater than at or about 95% of the CD57^{pos} cells in the composition comprise a heterologous nucleic acid(s) encoding a CAR. In some of any such embodiments, greater than at or about 50%, greater than at or about 60%, greater than at or about 70%, greater than at or about 80%, greater than at or about 90% or greater than at or about 95% of the CD57^{pos} cells in the composition comprise a heterologous nucleic acid(s) encoding an immunomodulator (e.g. cytokine, either secretable or membrane-bound as described). In some embodiments, the phenotype further includes the surface phenotype CD45^{pos}/CD3^{neg}/CD56^{pos}. In some of any such embodiments, greater than at or about 50%, greater than at or about 60%, greater than at or about 70%, greater than at or about 80%, greater than at or about 90% or greater than at or about 95% of the CD57^{pos} cells in the composition comprise a heterologous nucleic acid(s) encoding a CAR and an immunomodulator (e.g. cytokine, either secretable or membrane-bound as described). In some embodiments, the phenotype further includes the surface phenotype CD45^{pos}/CD3^{neg}/CD56^{pos}. In some of any such embodiments, greater than at or about 50%, greater than at or about 60%, greater than at or about 70%, greater than at or about 80%, greater than at or about 90% or greater than at or about 95% of the CD3^{neg}CD57^{pos} cells in the composition comprise a heterologous nucleic acid(s) encoding a CAR. In some of any such embodiments, greater than at or about 50%, greater than at or about 60%, greater than at or about 70%, greater than at or about 80%, greater than at or about 90% or greater than at or about 95% of the CD3^{neg}CD57^{pos} cells in the composition comprise a heterologous nucleic acid(s) encoding an immunomodulator (e.g. cytokine, either secretable or membrane-bound as described). In some of any such embodiments, greater than at or about 50%, greater than at or about 60%, greater than at or about 70%, greater than at or about 80%, greater than at or about 90% or greater than at or about 95% of the CD3^{neg}CD57^{pos} cells in the composition comprise a heterologous nucleic acid(s) encoding a CAR and an immunomodulator (e.g. cytokine, either secretable or membrane-bound as described). In some of any such embodiments, greater than at or about 50%, greater than at or about 60%, greater than at or about 70%, greater than at or about 80%, greater than at or about 90% or greater than at or about 95% of the CD57^{pos} CD45^{pos}/CD3^{neg}/CD56^{pos} cells in the composition comprise a heterologous nucleic acid(s) encoding a CAR. In some of any such embodiments, greater than at or about 50%, greater than at or about 60%, greater than at or about 70%, greater than at or about 80%, greater than at or about 90% or greater than at or about 95% of the CD57^{pos} CD45^{pos}/CD3^{neg}/CD56^{pos} cells in the composition comprise a heterologous nucleic acid(s) encoding an immunomodulator (e.g. cytokine, either secretable or membrane-bound as described). In some of any such embodiments, greater than at or about 50%, greater than at or about 60%, greater than at or about 70%, greater than at or about 80%, greater than at or about 90% or greater than at or about 95% of the CD57^{pos} CD45^{pos}/CD3^{neg}/CD56^{pos} cells in the composition comprise a heterologous nucleic acid(s) encoding a CAR and an immunomodulator (e.g. cytokine, either secretable or membrane-bound as described). In some of any of the provided embodiments, of the cells that have such a phenotype greater than 50% are FcRγ^{neg}, optionally between at or about 50% and 90% are FcRγ^{neg}. In some of any of the provided embodiments, of the cells that have such a phenotype greater than 70% are FcRγ^{neg}, optionally between at or about 70% and 90% are FcRγ^{neg}.

In some embodiments, the composition includes a population of a natural killer (NK) cell subset, wherein at least at or about 40%, at least at or about 50%, at least at or about 55%, at least at or about 60%, at least at or about 65%, at least at or about 70%, at least at or about 75%, at least at least at or about 80%, at least at or about 85%, at least at or about 90%, or at least at or about 95% of the cells in the composition have a g-NK cell surrogate marker profile that is CD16^{pos}/CD57^{pos}/CD7^{dim/neg}/CD161^{neg}. In some embodiments, from or from about 70% to at or about 90% of the cells in the composition have the phenotype CD16^{pos}/CD57^{pos}/CD7^{dim/neg}/CD161^{neg}. In some embodiments, at least at or about 72%, at least at or about 74%, at least at or about 76%, at least at or about 78%, at least at or about 80%, at least at or about 82%, at least at or about 84%, at least at or about 86%, at least at or about 88%, at least at or about 90%, at least at or about 92%, at least at or about 94%, at least at or about 96% or at least at or about 98% of cell in the composition have the phenotype CD16^{pos}/CD57^{pos}/CD7^{dim/neg}/CD161^{neg}. In some of any of the provided embodiments, at least at or about 60% of the cells in the composition comprise the phenotype. CD16^{pos}/CD57^{pos}/CD7^{dim/neg}/CD161^{neg}. In some of any of the provided embodiments, at least at or about 70% of the cells in the composition comprise the phenotype CD16^{pos}/CD57^{pos}/CD7^{dim/neg}/CD161^{neg}. In some of any such embodiments, greater than at or about 50%, greater than at or about 60%, greater than at or about 70%, greater than at or about 80%, greater than at or about 90% or greater than at or about 95% of the CD16^{pos}/CD57^{pos}/CD7^{dim/neg}/CD161^{neg} cells in the composition comprise a heterologous nucleic acid(s) encoding a CAR. In some of any such embodiments, greater than at or about 50%, greater than at or about 60%, greater than at or about 70%, greater than at or about 80%, greater than at or about 90% or greater than at or about 95% of the CD16^{pos}/CD57^{pos}/CD7^{dim/neg}/CD161^{neg} cells in the composition comprise a heterologous nucleic acid(s) encoding an immunomodulator (e.g. cytokine, either secretable or membrane-bound as described). In some of any such embodiments, greater than at or about 50%, greater than at or about 60%, greater than at or about 70%, greater than at or about 80%, greater than at or about 90% or greater than at or about 95% of the CD16^{pos}/CD57^{pos}/CD7^{dim/neg}/CD161^{neg} cells in the composition comprise a heterologous nucleic acid(s) encoding a CAR and an immunomodulator (e.g. cytokine, either secretable or membrane-bound as described). In some embodiments, the phenotype further includes the surface phenotype CD3^{neg}. In some embodiments, the phenotype further includes the surface phenotype CD45^{pos}/CD3^{neg}/CD56^{pos}. In some of any of the provided embodiments, of the cells that have such a phenotype greater than 50% are FcRγ^{neg}, optionally between at or about 50% and 90% are FcRγ^{neg}. In some of any of the provided embodiments, of the cells that have such a phenotype greater than 70% are FcRγ^{neg}, optionally between at or about 70% and 90% are FcRγ^{neg}.

In some embodiments, the composition includes a population of a natural killer (NK) cell subset, wherein at least at or about 40%, at least at or about 50%, at least at or about 55%, at least at or about 60%, at least at or about 65%, at least at or about 70%, at least at or about 75%, at least at least at or about 80%, at least at or about 85%, at least at or about 90%, or at least at or about 95% of the cells in the composition have a phenotype that is CD38^{neg}. In some embodiments, from or from about 70% to at or about 90% of the cells in the composition have the phenotype CD38^{neg}. In some embodiments, at least at or about 72%, at least at or about 74%, at least at or about 76%, at least at or about 78%, at least at or about 80%, at least at or about 82%, at least at or about 84%, at least at or about 86%, at least at or about 88%, at least at or about 90%, at least at or about 92%, at least at or about 94%, at least at or about 96% or at least at or about 98% of cell in the composition have the phenotype CD38^{neg}. In some of any of the provided embodiments, at least at or about 60% of the cells in the composition comprise the phenotype CD38^{neg}. In some of any of the provided embodiments, at least at or about 70% of the cells in the composition comprise the phenotype CD38^{neg}. In some embodiments, the phenotype further includes the surface phenotype CD3^{neg}. In some embodiments, the phenotype further includes the surface phenotype CD45^{pos}/CD3^{neg}/CD56^{pos}. In some of any such embodiments, greater than at or about 50%, greater than at or about 60%, greater than at or about 70%, greater than at or about 80%, greater than at or about 90% or greater than at or about 95% of the CD38^{neg} cells in the composition comprise a heterologous nucleic acid(s) encoding a CAR and an immunomodulator (e.g. cytokine, either secretable or membrane-bound as described). In some of any such embodiments, greater than at or about 50%, greater than at or about 60%, greater than at or about 70%, greater than at or about 80%, greater than at or about 90% or greater than at or about 95% of the CD3^{neg} CD38^{neg} cells in the composition comprise a heterologous nucleic acid(s) encoding a CAR. In some of any such embodiments, greater than at or about 50%, greater than at or about 60%, greater than at or about 70%, greater than at or about 80%, greater than at or about 90% or greater than at or about 95% of the CD3^{neg} CD38^{neg} cells in the composition comprise a heterologous nucleic acid(s) encoding an immunomodulator (e.g. cytokine, either secretable or membrane-bound as described). In some of any such embodiments, greater than at or about 50%, greater than at or about 60%, greater than at or about 70%, greater than at or about 80%, greater than at or about 90% or greater than at or about 95% of the CD38^{neg} cells in the composition comprise heterologous nucleic acid(s) encoding a CAR and an immunomodulator (e.g. cytokine, either secretable or membrane-bound as described). In some of any such embodiments, greater than at or about 50%, greater than at or about 60%, greater than at or about 70%, greater than at or about 80%, greater than at or about 90% or greater than at or about 95% of the CD38^{neg}CD45^{pos}/CD3^{neg}/CD56^{pos} cells in the composition comprise a heterologous nucleic acid(s) encoding a CAR. In some of any such embodiments, greater than at or about 50%, greater than at or about 60%, greater than at or about 70%, greater than at or about 80%, greater than at or about 90% or greater than at or about 95% of the CD38^{neg}CD45^{pos}/CD3^{neg}/CD56^{pos} cells in the composition comprise a heterologous nucleic acid(s) encoding an immunomodulator (e.g. cytokine, either secretable or membrane-bound as described). In some of any such embodiments, greater than at or about 50%, greater than at or about 60%, greater than at or about 70%, greater than at or about 80%, greater than at or about 90% or greater than at or about 95% of the CD38^{neg}CD45^{pos}/CD3^{neg}/CD56^{pos} cells in the composition comprise heterologous nucleic acid(s) encoding a CAR and an immunomodulator (e.g. cytokine, either secretable or membrane-bound as described). In some of any of the provided embodiments, of the cells that have such a phenotype greater than 50% are FcRγ^{neg}, optionally between at or about 50% and 90% are FcRγ^{neg}. In some of any of the provided embodiments, of the cells that have such a phenotype greater than 70% are FcRγ^{neg}, optionally between at or about 70% and 90% are FcRγ^{neg}.

In some embodiments, the composition includes a population of a natural killer (NK) cell subset, wherein at least at or about 40%, at least at or about 50%, at least at or about 55%, at least at or about 60%, at least at or about 65%, at least at or about 70%, at least at or about 75%, at least at least at or about 80%, at least at or about 85%, at least at or about 90%, or at least at or about 95% of the cells in the composition have a phenotype that is CD16^{pos}. In some embodiments, from or from about 70% to at or about 90% of the cells in the composition have the phenotype CD16^{pos}. In some embodiments, at least at or about 72%, at least at or about 74%, at least at or about 76%, at least at or about 78%, at least at or about 80%, at least at or about 82%, at least at or about 84%, at least at or about 86%, at least at or about 88%, at least at or about 90%, at least at or about 92%, at least at or about 94%, at least at or about 96% or at least at or about 98% of cell in the composition have the phenotype CD16^{pos}. In some of any of the provided embodiments, at least at or about 60% of the cells in the composition comprise the phenotype CD16^{pos}. In some of any of the provided embodiments, at least at or about 70% of the cells in the composition comprise the phenotype CD16^{pos}. In some embodiments, the phenotype further includes the surface phenotype CD3^{neg}. In some embodiments, the phenotype further includes the surface phenotype CD45^{pos}/CD3^{neg}/CD56^{pos}. In some of any such embodiments, greater than at or about 50%, greater than at or about 60%, greater than at or about 70%, greater than at or about 80%, greater than at or about 90% or greater than at or about 95% of the CD16^{pos} cells in the composition comprise a heterologous nucleic acid(s) encoding a CAR. In some of any such embodiments, greater than at or about 50%, greater than at or about 60%, greater than at or about 70%, greater than at or about 80%, greater than at or about 90% or greater than at or about 95% of the CD16^{pos} cells in the composition comprise a heterologous nucleic acid(s) encoding an immunomodulator (e.g. cytokine, either secretable or membrane-bound as described). In some of any such embodiments, greater than at or about 50%, greater than at or about 60%, greater than at or about 70%, greater than at or about 80%, greater than at or about 90% or greater than at or about 95% of the CD16^{pos} cells in the composition comprise heterologous nucleic acid(s) encoding a CAR and an immunomodulator (e.g. cytokine, either secretable or membrane-bound as described). In some of any such embodiments, greater than at or about 50%, greater than at or about 60%, greater than at or about 70%, greater than at or about 80%, greater than at or about 90% or greater than at or about 95% of the CD3^{neg} CD16^{pos} cells in the composition comprise a heterologous nucleic acid(s) encoding a CAR. In some of any such embodiments, greater than at or about 50%, greater than at or about 60%, greater than at or about 70%, greater than at or about 80%, greater than at or about 90% or greater than at or about 95% of the CD3^{neg} CD16^{pos} cells in the composition comprise a heterologous nucleic acid(s) encoding an immunomodulator (e.g. cytokine, either secretable or membrane-bound as described). In some of any such embodiments, greater than at or about 50%, greater than at or about 60%, greater than at or about 70%, greater than at or about 80%, greater than at or about 90% or greater than at or about 95% of the CD3^{neg CD}16^{pos} cells in the composition comprise heterologous nucleic acid(s) encoding a CAR and an immunomodulator (e.g. cytokine, either secretable or membrane-bound as described). In some of any such embodiments, greater than at or about 50%, greater than at or about 60%, greater than at or about 70%, greater than at or about 80%, greater than at or about 90% or greater than at or about 95% of the CD16^{pos} CD45^{pos}/CD3^{neg}/CD56^{pos} cells in the composition comprise a heterologous nucleic acid(s) encoding a CAR. In some of any such embodiments, greater than at or about 50%, greater than at or about 60%, greater than at or about 70%, greater than at or about 80%, greater than at or about 90% or greater than at or about 95% of the CD16^{pos} CD45^{pos}/CD3^{neg}/CD56^{pos} cells in the composition comprise a heterologous nucleic acid(s) encoding an immunomodulator (e.g. cytokine, either secretable or membrane-bound as described). In some of any such embodiments, greater than at or about 50%, greater than at or about 60%, greater than at or about 70%, greater than at or about 80%, greater than at or about 90% or greater than at or about 95% of the CD16^{pos} CD45^{pos}/CD3^{neg}/CD56^{pos} cells in the composition comprise heterologous nucleic acid(s) encoding a CAR and an immunomodulator (e.g. cytokine, either secretable or membrane-bound as described). In some of any of the provided embodiments, of the cells that have such a phenotype greater than 50% are FcRγ^{neg}, optionally between at or about 50% and 90% are FcRγ^{neg}. In some of any of the provided embodiments, of the cells that have such a phenotype greater than 70% are FcRγ^{neg}, optionally between at or about 70% and 90% are FcRγ^{neg}.

In some embodiments, the composition includes a population of a natural killer (NK) cell subset, wherein at least at or about 40%, at least at or about 50%, at least at or about 55%, at least at or about 60%, at least at or about 65%, at least at or about 70%, at least at or about 75%, at least at least at or about 80%, at least at or about 85%, at least at or about 90%, or at least at or about 95% of the cells in the composition have a g-NK cell surrogate marker profile that is NKG2A^{neg}/CD161^{neg}. In some embodiments, from or from about 70% to at or about 90% of the cells in the composition have the phenotype NKG2A^{neg}/CD161^{neg}. In some embodiments, at least at or about 72%, at least at or about 74%, at least at or about 76%, at least at or about 78%, at least at or about 80%, at least at or about 82%, at least at or about 84%, at least at or about 86%, at least at or about 88%, at least at or about 90%, at least at or about 92%, at least at or about 94%, at least at or about 96% or at least at or about 98% of cell in the composition have the phenotype NKG2A^{neg}/CD161^{neg}. In some of any of the provided embodiments, at least at or about 60% of the cells in the composition comprise the phenotype NKG2A^{neg}/CD161^{neg}. In some of any of the provided embodiments, at least at or about 70% of the cells in the composition comprise the phenotype NKG2A^{neg}/CD161^{neg}. In some embodiments, the phenotype further includes the surface phenotype CD3^{neg}. In some embodiments, the phenotype further includes the surface phenotype CD45^{pos}/CD3^{neg}/CD56^{pos}. In some of any such embodiments, greater than at or about 50%, greater than at or about 60%, greater than at or about 70%, greater than at or about 80%, greater than at or about 90% or greater than at or about 95% of the NKG2A^{neg}/CD161^{neg} cells in the composition comprise a heterologous nucleic acid(s) encoding a CAR. In some of any such embodiments, greater than at or about 50%, greater than at or about 60%, greater than at or about 70%, greater than at or about 80%, greater than at or about 90% or greater than at or about 95% of the NKG2A^{neg}/CD161^{neg} cells in the composition comprise comprise a heterologous nucleic acid(s) encoding an immunomodulator, such as a cytokine. In some of any such embodiments, greater than at or about 50%, greater than at or about 60%, greater than at or about 70%, greater than at or about 80%, greater than at or about 90% or greater than at or about 95% of the NKG2A^{neg}/CD161^{neg} cells in the composition comprise heterologous nucleic acid(s) encoding an antigen receptor, such as a CAR, and an immunomodulator, such as a cytokine. In some of any such embodiments, greater than at or about 50%, greater than at or about 60%, greater than at or about 70%, greater than at or about 80%, greater than at or about 90% or greater than at or about 95% of the CD3^{neg} NKG2A^{neg}/CD161^{neg} cells in the composition comprise a heterologous nucleic acid(s) encoding a CAR In some of any such embodiments, greater than at or about 50%, greater than at or about 60%, greater than at or about 70%, greater than at or about 80%, greater than at or about 90% or greater than at or about 95% of the CD3^{neg} NKG2A^{neg}/CD161^{neg} cells in the composition comprise a heterologous nucleic acid(s) encoding an immunomodulator, such as a cytokine. In some of any such embodiments, greater than at or about 50%, greater than at or about 60%, greater than at or about 70%, greater than at or about 80%, greater than at or about 90% or greater than at or about 95% of the CD3^{neg} NKG2A^{neg}/CD161^{neg} cells in the composition comprise heterologous nucleic acid(s) encoding an antigen receptor, such as a CAR, and an immunomodulator, such as a cytokine. In some of any such embodiments, greater than at or about 50%, greater than at or about 60%, greater than at or about 70%, greater than at or about 80%, greater than at or about 90% or greater than at or about 95% of the NKG2A^{neg}/CD161^{neg}/CD45^{pos}/CD3^{neg}/CD56^{pos} cells in the composition comprise a heterologous nucleic acid(s) encoding a CAR. In some of any such embodiments, greater than at or about 50%, greater than at or about 60%, greater than at or about 70%, greater than at or about 80%, greater than at or about 90% or greater than at or about 95% of the NKG2A^{neg}/CD161^{neg}/CD45^{pos}/CD3^{neg}/CD56^{pos} cells in the composition comprise a heterologous nucleic acid(s) encoding an immunomodulator, such as a cytokine. In some of any such embodiments, greater than at or about 50%, greater than at or about 60%, greater than at or about 70%, greater than at or about 80%, greater than at or about 90% or greater than at or about 95% of the NKG2A^{neg}/CD161^{neg}/CD45^{pos}/CD3^{neg}/CD56^{pos} cells in the composition comprise heterologous nucleic acid(s) encoding an antigen receptor, such as a CAR, and an immunomodulator, such as a cytokine. In some of any of the provided embodiments, of the cells that have such a phenotype greater than 50% are FcRγ^{neg}, optionally between at or about 50% and 90% are FcRγ^{neg}. In some of any of the provided embodiments, of the cells that have such a phenotype greater than 70% are FcRγ^{neg}, optionally between at or about 70% and 90% are FcRγ^{neg}.

In some embodiments, the composition includes a population of NK cells wherein greater than at or about 50% of the NK cells in the composition are g-NK cells (FcRγ^{neg}) or NK cells expressing a surrogate marker profile thereof. In some embodiments, the composition includes a population of NK cells wherein greater than at or about 55% of the NK cells in the composition are g-NK cells (FcRγ^{neg}) or NK cells expressing a surrogate marker profile thereof. In some embodiments, the composition includes a population of NK cells wherein greater than at or about 60% of the NK cells in the composition are g-NK cells (FcRγ^{neg}) or NK cells expressing a surrogate marker profile thereof. In some embodiments, the composition includes a population of NK cells wherein greater than at or about 65% of the NK cells in the composition are g-NK cells (FcRγ^{neg}) or NK cells expressing a surrogate marker profile thereof. In some embodiments, the composition includes a population of NK cells wherein greater than at or about 70% of the NK cells in the composition are g-NK cells (FcRγ m) or NK cells expressing a surrogate marker profile thereof. In some embodiments, the composition includes a population of NK cells wherein greater than at or about 75% of the NK cells in the composition are g-NK cells (FcRγ^{neg}) or NK cells expressing a surrogate marker profile thereof. In some embodiments, the composition includes a population of NK cells wherein greater than at or about 80% of the NK cells in the composition are g-NK cells (FcRγ^{neg}) or NK cells expressing a surrogate marker profile thereof. In some embodiments, the composition includes a population of NK cells wherein greater than at or about 85% of the NK cells in the composition are g-NK cells (FcRγ^{neg}) or NK cells expressing a surrogate marker profile thereof. In some embodiments, the composition includes a population of NK cells wherein greater than at or about 90% of the NK cells in the composition are g-NK cells (FcRγ^{neg}) or NK cells expressing a surrogate marker profile thereof. In some embodiments, the composition includes a population of NK cells wherein greater than at or about 95% of the NK cells in the composition are g-NK cells (FcRγ m) or NK cells expressing a surrogate marker profile thereof. The surrogate marker profile may be any as described herein. For example, the surrogate marker profile may be CD16^{pos}/CD57^{pos}/CD7^{dim/neg}/CD161^{neg}. In other examples, the surrogate marker profile may be NKG2A^{neg}/CD161^{neg}. In further examples, the g-NK cell surrogate marker profile is CD38^{neg}. A surrogate surface marker profile may further include the phenotype CD45^{pos}/CD3^{neg}/CD56^{pos}.

In some embodiments, the g-NK cells of the composition, or a certain percentage thereof, e.g. greater than about 70%, are positive for perforin and/or granzyme B. In some embodiments, natural killer cells in the composition are enriched in cells that are positive for perforin and granzyme B. In some cases, natural killer cells are positive for perforin and granzyme B. Perforin is a pore forming cytolytic protein found in the granules of NK cells. Upon degranulation, perforin binds to the target cell's plasma membrane and oligomerizes in a calcium-dependent manner to form pores on the target cells. Granzyme B is a serine protease most commonly found in the granules of natural killer cells and cytotoxic T cells. Granzyme B is secreted with perforin to mediate apoptosis in target cells. Methods for measuring the number of cells positive for perforin or granzyme B are known to a skilled artisan. Methods include, for example, intracellular flow cytometry. In an example, the percentage or number of cells positive for perforin or granzyme B may be determined by the permeabilization of cells, for instance using the Inside Stain Kit from Miltenyi Biotec, prior to staining with antibodies against perforin and granzyme B. Cell staining can then be resolved for instance using flow cytometry.

In some embodiments, greater than at or about 70% of the g-NK cells of the composition are positive for perforin, and greater than at or about 70% of the g-NK cells of the composition are positive for granzyme B. In some embodiments, greater than at or about 75% of the g-NK cells of the composition are positive for perforin, and greater than at or about 75% of the g-NK cells of the composition are positive for granzyme B. In some embodiments, greater than at or about 80% of the g-NK cells of the composition are positive for perforin, and greater than at or about 80% of the g-NK cells of the composition are positive for granzyme B. In some embodiments, greater than at or about 85% of the g-NK cells of the composition are positive for perforin, and greater than at or about 85% of the g-NK cells of the composition are positive for granzyme B. In some embodiments, greater than at or about 90% of the g-NK cells of the composition are positive for perforin, and greater than at or about 90% of the g-NK cells of the composition are positive for granzyme B. In some embodiments, greater than at or about 95% of the g-NK cells of the composition are positive for perforin, and greater than at or about 95% of the g-NK cells of the composition are positive for granzyme B. In some of any such embodiments, greater than at or about 50%, greater than at or about 60%, greater than at or about 70%, greater than at or about 80%, greater than at or about 90% or greater than at or about 95% of the cells positive for granzyme B and perforin in the composition comprise a heterologous nucleic acid(s) encoding a CAR. In some of any such embodiments, greater than at or about 50%, greater than at or about 60%, greater than at or about 70%, greater than at or about 80%, greater than at or about 90% or greater than at or about 95% of the cells positive for granzyme B and perforin in the composition comprise a heterologous nucleic acid(s) encoding an immunomodulator (e.g. cytokine, either secretable or membrane-bound as described). In some of any such embodiments, greater than at or about 50%, greater than at or about 60%, greater than at or about 70%, greater than at or about 80%, greater than at or about 90% or greater than at or about 95% of the cells positive for granzyme B and perforin in the composition comprise heterologous nucleic acid(s) encoding an antigen receptor, such as a CAR, and an immunomodulator (e.g. cytokine, either secretable or membrane-bound as described).

In some embodiments, perforin and granzyme B expression levels by NK cells, for instance g-NK cells, can be measured by intracellular flow cytometry and levels measured based on levels of mean fluorescence intensity (MFI). In some embodiments, perforin and granzyme B expression levels based on MFI will differ between g-NK cells and cells that are FcRγ^{pos}. In some embodiments, the g-NK cells of the composition that are positive for perforin express a mean level of perforin, based on MFI levels, at least at or about two times the mean level of perforin expressed by FcRγ^{pos} NK cells. In some embodiments, the g-NK cells of the composition that are positive for perforin express a mean level of perforin, based on MFI levels, at least at or about three times the mean level of perforin expressed by FcRγ^{pos} NK cells. In some embodiments, the g-NK cells of the composition that are positive for perforin express a mean level of perforin, based on MFI levels, at least at or about four times the mean level of perforin expressed by FcRγ^{pos} NK cells. In some embodiments, the g-NK cells of the composition that are positive for granzyme B express a mean level of granzyme B, based on MFI levels, at least at or about two times the mean level of granzyme B expressed by FcRγ^{pos} NK cells. In some embodiments, the g-NK cells of the composition that are positive for granzyme B express a mean level of granzyme B, based on MFI levels, at least at or about three times the mean level of granzyme B expressed by FcRγ^{pos} NK cells. In some embodiments, the g-NK cells of the composition that are positive for granzyme B express a mean level of granzyme B, based on MFI levels, at least at or about four times the mean level of granzyme B expressed by FcRγ^{pos} NK cells.

In some embodiments, at least at or about 50% of the cells in the composition are FcRγ-deficient NK cells (g-NK), wherein greater than at or about 70% of the g-NK cells are positive for perforin and greater than at or about 70% of the g-NK cells are positive for granzyme B. In some embodiments, greater than at or about 80% of the g-NK cells are positive for perforin and greater than at or about 80% of the g-NK cells are positive for granzyme B. In some embodiments, greater than at or about 90% of the g-NK cells are positive for perforin and greater than at or about 90% of the g-NK cells are positive for granzyme B. In some embodiments, greater than at or about 95% of the g-NK cells are positive for perforin and greater than at or about 95% of the g-NK cells are positive for granzyme B. In some embodiments, the g-NK cells are FcRγ^{neg}. In some of any such embodiments, greater than at or about 50%, greater than at or about 60%, greater than at or about 70%, greater than at or about 80%, greater than at or about 90% or greater than at or about 95% of the cells positive for perforin and granzyme B in the composition comprise a heterologous nucleic acid(s) encoding a CAR. In some embodiments, the g-NK cells are FcRγ^{neg}. In some of any such embodiments, greater than at or about 50%, greater than at or about 60%, greater than at or about 70%, greater than at or about 80%, greater than at or about 90% or greater than at or about 95% of the cells positive for perforin and granzyme B in the composition comprise a heterologous nucleic acid(s) encoding an immunomodulator (e.g. cytokine, either secretable or membrane-bound as described). In some embodiments, the g-NK cells are FcRγ^{neg}. In some of any such embodiments, greater than at or about 50%, greater than at or about 60%, greater than at or about 70%, greater than at or about 80%, greater than at or about 90% or greater than at or about 95% of the cells positive for perforin and granzyme B in the composition comprise heterologous nucleic acid(s) encoding an antigen receptor, such as a CAR, and an immunomodulator (e.g. cytokine, either secretable or membrane-bound as described).

In some of any embodiments, among the cells positive for perforin, the cells express a mean level of perforin as measured by intracellular flow cytometry that is, based on mean fluorescence intensity (MFI), at least at or about two times the mean level of perforin expressed by cells that are FcRγ^{pos}. In some of any embodiments, among the cells positive for granzyme B, the cells express a mean level of granzyme B as measured by intracellular flow cytometry that is, based on mean fluorescence intensity (MFI), at least at or about two times the mean level of granzyme B expressed by cells that are FcRγ^{pos}.

In some embodiments, natural killer cells in the composition are enriched in cells that are express or produce CD107A, IFNγ, and TNF-α. In some cases, the expression or production, or a certain degree of expression or production, of such factors in in the absence of target antigen (i.e. is intrinsic to cells in the composition without further stimulation). In some cases, the expression or production, or a certain degree of expression or production, as in the presence of cells expressing a target antigen (target cells) and an antibody directed against the target antigen (anti-target antibody). In some embodiments, for instance, the target cells may be a tumor cell line expressing CD38 and the antibody is an anti-CD38 antibody (e.g. daratumumab). In some embodiments, for instance, the target cells may be a tumor cell line expressing CD20 and the antibody is an anti-CD20 antibody (e.g. rituximab).

In some of any embodiments, greater than 10% of the cells in the composition are capable of degranulation against tumor target cells, optionally as measured by CD107a expression, optionally wherein the degranulation is measured in the absence of an antibody against the tumor target cells. In some of any embodiments, among the cells in the composition, greater than at or about 15%, greater than at or about 20%, greater than at or about 30%, greater than at or about 40% or greater than at or about 50% exhibit degranulation, optionally as measured by CD107a expression, in the presence of cells expressing a target antigen (target cells) and an antibody directed against the target antigen (anti-target antibody). In some of any such embodiments, greater than 10% of the cells in the composition are further capable of producing interferon-gamma or TNF-alpha against tumor target cells, optionally wherein the interferon-gamma or TNF-alpha is measured in the absence of an antibody against the tumor target cells. In some of any such embodiments, greater than at or about 50%, greater than at or about 60%, greater than at or about 70%, greater than at or about 80%, greater than at or about 90% or greater than at or about 95% of the cells positive for CD107a in the composition, e.g. as measured in the presence or absence of cells expressing a target antigen and an antibody directed against the target antigen, comprise a heterologous nucleic acid(s) encoding a CAR. In some of any such embodiments, greater than at or about 50%, greater than at or about 60%, greater than at or about 70%, greater than at or about 80%, greater than at or about 90% or greater than at or about 95% of the cells positive for CD107a in the composition, e.g. as measured in the presence or absence of cells expressing a target antigen and an antibody directed against the target antigen, comprise a heterologous nucleic acid(s) encoding an immunomodulator (e.g. cytokine, either secretable or membrane-bound as described). In some of any such embodiments, greater than at or about 50%, greater than at or about 60%, greater than at or about 70%, greater than at or about 80%, greater than at or about 90% or greater than at or about 95% of the cells positive for CD107a in the composition, e.g. as measured in the presence or absence of cells expressing a target antigen and an antibody directed against the target antigen, comprise heterologous nucleic acid(s) encoding an antigen receptor, such as a CAR, and an immunomodulator (e.g. cytokine, either secretable or membrane-bound as described). In some embodiments, among the cells in the composition, greater than at or about 15%, greater than at or about 20%, greater than at or about 30%, greater than at or about 40% or greater than at or about 50% produce an effector cytokine in the presence of cells expressing a target antigen (target cells) and an antibody directed against the target antigen (anti-target antibody). In some embodiments, for instance, the target cells may be a tumor cell line expressing CD38 and the antibody is an anti-CD38 antibody (e.g. daratumumab). In some embodiments, for instance, the target cells may be a tumor cell line expressing CD20 and the antibody is an anti-CD20 antibody (e.g. rituximab).

In some embodiments, at least at or about 50% of the cells in the composition are FcRγ-deficient (FcRγ^{neg}) NK cells (g-NK), and wherein greater than at or about 15% of the cells in the composition produce an effector cytokine in the presence of cells expressing a target antigen (target cells) and an antibody directed against the target antigen (anti-target antibody). In some embodiments, greater than at or about 20%, greater than at or about 30%, greater than at or about 40% or greater than at or about 50% produce an effector cytokine in the presence of cells expressing a target antigen (target cells) and an antibody directed against the target antigen (anti-target antibody). In some embodiments, for instance, the target cells may be a tumor cell line expressing CD38 and the antibody is an anti-CD38 antibody (e.g. daratumumab). In some embodiments, for instance, the target cells may be a tumor cell line expressing CD20 and the antibody is an anti-CD20 antibody (e.g. rituximab). In some of any embodiments, the effector cytokine is IFN-gamma or TNF-alpha. In some of any embodiments, the effector cytokine is IFN-gamma and TNF-alpha. In some of any such embodiments, greater than at or about 50%, greater than at or about 60%, greater than at or about 70%, greater than at or about 80%, greater than at or about 90% or greater than at or about 95% of the cells that produce an effect cytokine (e.g. IFN-gamma or TNF-alpha) in the composition, e.g. as measured in the presence or absence of cells expressing a target antigen and an antibody directed against the target antigen, comprise a heterologous nucleic acid(s) encoding a CAR. In some of any embodiments, the effector cytokine is IFN-gamma and TNF-alpha. In some of any such embodiments, greater than at or about 50%, greater than at or about 60%, greater than at or about 70%, greater than at or about 80%, greater than at or about 90% or greater than at or about 95% of the cells that produce an effect cytokine (e.g. IFN-gamma or TNF-alpha) in the composition, e.g. as measured in the presence or absence of cells expressing a target antigen and an antibody directed against the target antigen, comprise a heterologous nucleic acid(s) encoding an immunomodulator (e.g. cytokine, either secretable or membrane-bound as described). In some of any embodiments, the effector cytokine is IFN-gamma and TNF-alpha. In some of any such embodiments, greater than at or about 50%, greater than at or about 60%, greater than at or about 70%, greater than at or about 80%, greater than at or about 90% or greater than at or about 95% of the cells that produce an effect cytokine (e.g. IFN-gamma or TNF-alpha) in the composition, e.g. as measured in the presence or absence of cells expressing a target antigen and an antibody directed against the target antigen, comprise heterologous nucleic acid(s) encoding an antigen receptor, such as a CAR, and an immunomodulator (e.g. cytokine, either secretable or membrane-bound as described).

In some of any embodiments, among the cells in the composition, greater than at or about 15%, greater than at or about 20%, greater than at or about 30%, greater than at or about 40% or greater than at or about 50% exhibit degranulation, optionally as measured by CD107a expression, in the presence of cells expressing a target antigen (target cells) and an antibody directed against the target antigen (anti-target antibody). In some embodiments, for instance, the target cells may be a tumor cell line expressing CD38 and the antibody is an anti-CD38 antibody (e.g. daratumumab). In some embodiments, for instance, the target cells may be a tumor cell line expressing CD20 and the antibody is an anti-CD20 antibody (e.g. rituximab).

In some embodiments, at least at or about 50% of the cells in the composition are FcRγ-deficient (FcRγ^{neg}) NK cells (g-NK), and wherein greater than at or about 15% of the cells in the composition exhibit degranulation, optionally as measured by CD107a expression, in the presence of cells expressing a target antigen (target cells) and an antibody directed against the target antigen (anti-target antibody). In some embodiments, greater than at or about 20%, greater than at or about 30%, greater than at or about 40% or greater than at or about 50% exhibit degranulation, optionally as measured by CD107a expression, in the presence of cells expressing a target antigen (target cells) and an antibody directed against the target antigen (anti-target antibody). In some embodiments, for instance, the target cells may be a tumor cell line expressing CD38 and the antibody is an anti-CD38 antibody (e.g. daratumumab). In some embodiments, for instance, the target cells may be a tumor cell line expressing CD20 and the antibody is an anti-CD20 antibody (e.g. rituximab).

In some of any of the provided embodiments greater than at or about 60% of the cells in the composition are g-NK cells. In some of any of the provided embodiments, greater than at or about 70% of the cells in the composition are g-NK cells. In some of any of the provided embodiments, greater than at or about 80% of the cells in the composition are g-NK cells. In some of any of the provided embodiments, greater than at or about 90% of the cells in the composition are g-NK cells. In some of any of the provided embodiments, greater than at or about 95% of the cells in the composition are g-NK cells.

In some embodiments, the g-NK cells exhibit a g-NK cell surrogate marker profile. In some embodiments, the g-NK cell surrogate marker profile is CD16pos/CD57^{pos}/CD7^{dim/neg}/CD161^{neg}. In some embodiments, the g-NK cell surrogate marker profile is NKG2A^{neg}/CD161^{neg}. In some embodiments, the g-NK cell surrogate marker profile is CD38^{neg}. In some embodiments, the g-NK cell surrogate surface marker profile further is CD45^{pos}/CD3^{neg}/CD56^{pos}.

In some of any of the preceding embodiments, greater than at or about 60% of the cells are g-NK cells. In some of any of the preceding embodiments, greater than at or about 70% of the cells are g-NK cells. In some of any of the preceding embodiments, greater than at or about 80% of the cells are g-NK cells. In some of any of the preceding embodiments, greater than at or about 90% of the cells are g-NK cells. In some of any of the preceding embodiments, greater than at or about 95% of the cells are g-NK cells.

In some of any of the preceding embodiments, greater than at or at about 80% of the cells are positive for perforin. In some of any of the preceding embodiments, greater than at or at about 90% of the cells are positive for perforin. In some of any of the preceding embodiments, among the cells positive for perforin, the cells express a mean level of perforin as measured by intracellular flow cytometry that is, based on mean fluorescence intensity (MFI), at least at or about two times the mean level of perforin expressed by cells that are FcRγ^{pos}.

In some of any of the preceding embodiments, greater than at or at about 80% of the cells are positive for granzyme B. In some of any of the preceding embodiments, greater than at or at about 90% of the cells are positive for granzyme B. In some of any of the preceding embodiments, among the cells positive for granzyme B, the cells express a mean level of granzyme B as measured by intracellular flow cytometry that is, based on mean fluorescence intensity (MFI), at least at or about two times the mean level of granzyme B expressed by cells that are FcRγ^{pos}.

In some of any of the provided embodiments, the composition comprises from at or about 10⁶ cells to at or about 10¹² cells. In some of any of the provided embodiments, the composition comprises from at or about 10⁶ to at or about 10¹¹ cells, from at or about 10⁶ to at or about 10¹⁰ cells, from at or about 10⁶ to at or about 10¹¹ cells, from at or about 10⁶ to at or about 10⁸ cells, from at or about 10⁶ to at or about 10⁷ cells, from at or about 10⁷ to at or about 10¹² cells, from at or about 10⁷ to at or about 10¹¹ cells, from at or about 10⁷ to at or about 10¹⁰ cells, from at or about 10⁷ to at or about 10⁹ cells, or from at or about 10⁷ to at or about 10⁸ cells, from at or about 10⁸ to at or about 10¹² cells, from at or about 10⁸ to at or about 10¹¹ cells, from at or about 10⁸ to at or about 10¹⁰ cells, from at or about 10⁸ to at or about 10⁹ cells, from at or about 10⁹ to at or about 10¹² cells, from at or about 10⁹ to at or about 10¹¹ cells, from at or about 10⁹ to at or about 10¹⁰ cells, from at or about 10¹⁰ to at or about 10¹² cells, from at or about 10¹⁰ to at or about 10¹¹ cells, or from at or about 10¹¹ to at or about 10¹² cells.

In some of any of the provided embodiments, the composition comprises at least or about at least 10⁶ cells. In some of any of the provided embodiments, the composition comprises from at or about 10⁶ to at or about 10¹⁰ cells, from at or about 10⁶ to at or about 10⁹ cells, from at or about 10⁶ to at or about 10⁸ cells, from at or about 10⁶ to at or about 10⁷ cells, from at or about 10⁷ to at or about 10¹⁰ cells, from at or about 10⁷ to at or about 10⁹ cells, from at or about 10⁷ to at or about 10⁸ cells, from at or about 10⁸ to at or about 10¹⁰ cells, from at or about 10⁸ to at or about 10⁹ cells, or from at or about 10⁹ to at or about 10¹⁰ cells.

In some of any of the provided embodiments, the composition comprises at least or about at least 10⁸ cells. In some of any of the provided embodiments, the composition comprises at least at or about 10⁹ cells. In some of any of the provided embodiments, the composition comprises at least at or about 10¹⁰ cells. In some of any of the provided embodiments, the composition comprises at least at or about 10¹¹ cells. In some of any of the provided embodiments, the composition comprises from at or about 10⁸ to at or about 10¹¹ cells. In some of any of the provided embodiments, the composition comprises from at or about 10⁸ to at or about 10¹⁰ cells. In some of any of the provided embodiments, the composition comprises from at or about 10⁸ to at or about 10⁹ cells. In some of any of the provided embodiments, the composition comprises from at or about 10⁹ to at or about 10¹¹ cells. In some of any of the provided embodiments, the composition comprises from at or about 10⁹ to at or about 10¹⁰ cells. In some of any of the provided embodiments, the composition comprises from at or about 10¹⁰ to at or about 10¹¹ cells.

In some of any of the provided embodiments, the composition comprises at least at or about 10⁶ g-NK cells. In some of any of the provided embodiments, the composition comprises from at or about 10⁶ to at or about 10¹⁰ g-NK cells, from at or about 10⁶ to at or about 10⁹ g-NK cells, from at or about 10⁶ to at or about 10⁸ g-NK cells, from at or about 10⁶ to at or about 10⁷ g-NK cells, from at or about 10⁷ to at or about 10¹⁰ g-NK cells, from at or about 10⁷ to at or about 10⁹ g-NK cells, from at or about 10⁷ to at or about 10⁸ g-NK cells, from at or about 10⁸ to at or about 10¹⁰ g-NK cells, from at or about 10⁸ to at or about 10⁹ g-NK cells, or from at or about 10⁹ to at or about 10¹⁰ g-NK cells. In some of any of the provided embodiments, the g-NK cells are FcRγ^{neg}. In some of any of the provided embodiments, the g-NK cells are cells having a g-NK surrogate surface marker profile. In some embodiments, the g-NK cell surrogate surface marker profile is CD16^{pos}/CD57^{pos}/CD7^{dim/neg}/CD161^{neg}. In some embodiments, the g-NK cell surrogate surface marker profile is NKG2A^{neg}/CD161^{neg}. In some of any of the provided embodiments, the g-NK cells or cells having a g-NK surrogate marker profile further include the surface phenotype CD45^{pos}/CD3^{neg}/CD56^{pos}. In some of any of the provided embodiments, the g-NK cells or cells having a g-NK surrogate marker profile further include the surface phenotype CD38^{neg}.

In particular embodiments of any of the provided compositions, the cells in the composition are from the same donor. As such, the compositions do not include a mixed population of cells from one or more different donors. As provided here, the methods of expansion result in high yield expansion of at or greater than 500-fold, at or greater than 600-fold, at or greater than 700-fold, at or greater than 800-fold, at or greater than 900-fold, at or greater than 1000-fold or more of certain NK cell subsets, particularly the g-NK cell subset or an NK cell subset that is associated with or includes a surrogate marker for g-NK cells, such as any of the NK cell subsets described above. In some of any embodiments, the increase is at or about 1000-fold greater. In some of any embodiments, the increase is at or about 2000-fold greater. In some of any embodiments, the increase is at or about 2500-fold greater. In some of any embodiments, the increase is at or about 3000-fold greater. In some of any embodiments, the increase is at or about 5000-fold greater. In some of any embodiments, the increase is at or about 10000-fold greater. In some of any embodiments, the increase is at or about 15000-fold greater. In some of any embodiments, the increase is at or about 20000-fold greater. In some of any embodiments, the increase is at or about 25000-fold greater. In some of any embodiments, the increase is at or about 30000-fold greater. In some of any embodiments, the increase is at or about 35000-fold greater. In particular embodiments, expansion results in at or about 1,000 fold increase in number of certain NK cell subsets, particularly the g-NK cell subset or an NK cell subset that is associated with or includes a surrogate marker for g-NK cells, such as any of the NK cell subsets described above. In particular embodiments, expansion results in at or about 3,000 fold increase in number of certain NK cell subsets, particularly the g-NK cell subset or an NK cell subset that is associated with or includes a surrogate marker for g-NK cells, such as any of the NK cell subsets described above. In particular embodiments, expansion results in at or about 35,000 fold increase in number of certain NK cell subsets, particularly the g-NK cell subset or an NK cell subset that is associated with or includes a surrogate marker for g-NK cells, such as any of the NK cell subsets described above.

In some cases, expansion achieved by the provided methods from an initial source of NK cells obtained from a single donor can produce a composition of cells to provide a plurality of individual doses for administration to a subject in need. As such, the provided methods are particularly suitable for allogeneic methods. In some cases, a single expansion from a starting population of NK cells isolated from one donor in accord with the provided methods can result in greater than or greater than about 20 individual doses for administration to a subject in need, such as at or about 30 individual doses, 40 individual doses, 50 individual doses, 60 individual doses, 70 individual doses, 80 individual doses, 90 individual doses, 100 individual doses, or an individual dose that is a value between any of the foregoing. In some embodiments, the individual dose is from at or about 1×10⁵ cells/kg to at or about 1×10⁷ cells/kg, such as from at or about 1×10⁵ cells/kg to at or about 7.5×10⁶ cells/kg, from at or about 1×10⁵ cells/kg to at or about 5×10⁶ cells/kg, from at or about 1×10⁵ cells/kg to at or about 2.5×10⁶ cells/kg, from at or about 1×10⁵ cells/kg to at or about 1×10⁶ cells/kg, from at or about 1×10⁵ cells/kg to at or about 7.5×10⁵ cells/kg, from at or about 1×10⁵ cells/kg to at or about 5×10⁵ cells/kg, from at or about 1×10⁵ cells/kg to at or about 2.5×10⁵ cells/kg, from at or about 2.5×10⁵ cells/kg to at or about 1×10⁷ cells/kg, from at or about 2.5×10⁵ cells/kg to at or about 7.5×10⁶ cells/kg, from at or about 2.5×10⁵ cells/kg to at or about 5×10⁶ cells/kg, from at or about 2.5×10⁵ cells/kg to at or about 2.5×10⁶ cells/kg, from at or about 2.5×10⁵ cells/kg to at or about 1×10⁶ cells/kg, from at or about 2.5×10⁵ cells/kg to at or about 7.5×10⁵ cells/kg, from at or about 2.5×10⁵ cells/kg to at or about 5×10⁵ cells/kg, from at or about 5×10⁵ cells/kg to at or about 1×10⁷ cells/kg, from at or about 5×10⁵ cells/kg to at or about 7.5×10⁶ cells/kg, from at or about 5×10⁵ cells/kg to at or about 5×10⁶ cells/kg, from at or about 5×10⁵ cells/kg to at or about 2.5×10⁶ cells/kg, from at or about 5×10⁵ cells/kg to at or about 1×10⁶ cells/kg, from at or about 5×10⁵ cells/kg to at or about 7.5×10⁵ cells/kg, from at or about 1×10⁶ cells/kg to at or about 1×10⁷ cells/kg, from at or about 1×10⁶ cells/kg to at or about 7.5×10⁶ cells/kg, from at or about 1×10⁶ cells/kg to at or about 5×10⁶ cells/kg, from at or about 1×10⁶ cells/kg to at or about 2.5×10⁶ cells/kg, from at or about 2.5×10⁶ cells/kg to at or about 1×10⁷ cells/kg, from at or about 2.5×10⁶ cells/kg to at or about 7.5×10⁶ cells/kg, from at or about 2.5×10⁶ cells/kg to at or about 5×10⁶ cells/kg, from at or about 5×10⁶ cells/kg to at or about 1×10⁷ cells/kg, from at or about 5×10⁶ cells/kg to at or about 7.5×10⁶ cells/kg, or from at or about 7.5×10⁶ cells/kg to at or about 1×10⁷ cells/kg. In some embodiments, the individual dose is from at or about 1×10⁵ cells/kg to at or about 1×10⁸ cells/kg, such as from at or about 2.5×10⁵ cells/kg to at or about 1×10⁸ cells/kg, from at or about 5×10⁵ cells/kg to at or about 1×10⁸ cells/kg, from at or about 7.5×10⁵ cells/kg to at or about 1×10⁸ cells/kg, from at or about 1×10⁶ cells/kg to at or about 1×10⁸ cells/kg, from at or about 2.5×10⁶ cells/kg to at or about 1×10⁸ cells/kg, from at or about 5×10⁶ cells/kg to at or about 1×10⁸ cells/kg, from at or about 7.5×10⁶ cells/kg to at or about 1×10⁸ cells/kg, from at or about 1×10⁷ cells/kg to at or about 1×10⁸ cells/kg, from at or about 2.5×10⁷ cells/kg to at or about 1×10⁸ cells/kg, from at or about 5×10⁷ cells/kg to at or about 1×10⁸ cells/kg, or from at or about 7.5×10⁷ cells/kg to at or about 1×10⁸ cells/kg. In some embodiments, the individual dose is from at or about 5×10⁷ to at or about 10×10⁹, such as from at or about 5×10⁷ to at or about 5×10⁹, from about or about 5×10⁷ to at or about 1×10⁹, from at or about 5×10⁷ to at or about 5×10⁸, from about or about 5×10⁷ to at or about 1×10⁸, 1×10⁸ to at or about 10×10⁹, from at or about 1×10⁸ to at or about 5×10⁹, from about or about 1×10⁸ to at or about 1×10⁹, from at or about 1×10⁸ to at or about 5×10⁸, from at or about 5×10⁸ to at or about 10×10⁹, from at or about 5×10⁸ to at or about 5×10⁹, from about or about 5×10⁸ to at or about 1×10⁹, from at or about 1×10⁹ to at or about 10×10⁹, from at or about 1×10⁹ to at or about 5×10⁹, or from at or about 5×10⁹ to at or about 10×10⁹. In some embodiments, the individual dose is or is about 5×10⁸ cells. In some embodiments, the individual dose is or is about 1×10⁹ cells. In some embodiments, the individual dose is or is about 5×10⁹ cells. In some embodiments, the individual dose is or is about 1×10¹⁰ cells. In any of the above embodiments, the dose is given as the number of cells g-NK cells or an NK cell subset that is associated with or includes a surrogate marker for g-NK cells, such as any of the NK cell subsets described above, or a number of viable cells of any of the foregoing. In any of the above embodiments, the dose is given as the number of cells in a composition of expanded cells produced by the method, or a number of viable cells of any of the foregoing.

Among the compositions are pharmaceutical compositions and formulations for administration, such as for adoptive cell therapy. In some embodiments, the engineered cells are formulated with a pharmaceutically acceptable carrier.

A pharmaceutically acceptable carrier can include all solvents, dispersion media, coatings, antibacterial and antifungal agents, isotonic and absorption delaying agents, and the like, compatible with pharmaceutical administration (Gennaro, 2000, Remington: The science and practice of pharmacy, Lippincott, Williams & Wilkins, Philadelphia, PA). Examples of such carriers or diluents include, but are not limited to, water, saline, Ringer's solutions, dextrose solution, and 5% human serum albumin. Liposomes and non-aqueous vehicles such as fixed oils may also be used. Supplementary active compounds can also be incorporated into the compositions. The pharmaceutical carrier should be one that is suitable for NK cells, such as a saline solution, a dextrose solution or a solution comprising human serum albumin.

In some embodiments, the pharmaceutically acceptable carrier or vehicle for such compositions is any non-toxic aqueous solution in which the NK cells can be maintained, or remain viable, for a time sufficient to allow administration of live NK cells. For example, the pharmaceutically acceptable carrier or vehicle can be a saline solution or buffered saline solution. The pharmaceutically acceptable carrier or vehicle can also include various bio materials that may increase the efficiency of NK cells. Cell vehicles and carriers can, for example, include polysaccharides such as methylcellulose (M. C. Tate, D. A. Shear, S. W. Hoffman, D. G. Stein, M. C. LaPlaca, Biomaterials 22, 1113, 2001, which is incorporated herein by reference in its entirety), chitosan (Suh J K F, Matthew H W T. Biomaterials, 21, 2589, 2000; Lahiji A, Sohrabi A, Hungerford D S, et al., J Biomed Mater Res, 51, 586, 2000, each of which is incorporated herein by reference in its entirety), N-isopropylacrylamide copolymer P (NIPAM-co-AA) (Y. H. Bae, B. Vernon, C. K. Han, S. W. Kim, J. Control. Release 53, 249, 1998; H. Gappa, M. Baudys, J. J. Koh, S. W. Kim, Y. H. Bae, Tissue Eng. 7, 35, 2001, each of which is incorporated herein by reference in its entirety), as well as Poly(oxyethylene)/poly(D,L-lactic acid-co-glycolic acid) (B. Jeong, K. M. Lee, A. Gutowska, Y. H. An, Biomacromolecules 3, 865, 2002, which is incorporated herein by reference in its entirety), P(PF-co-EG) (Suggs L J, Mikos A G. Cell Trans, 8, 345, 1999, which is incorporated herein by reference in its entirety), PEO/PEG (Mann B K, Gobin A S, Tsai A T, Schmedlen R H, West J L., Biomaterials, 22, 3045, 2001; Bryant S J, Anseth K S. Biomaterials, 22, 619, 2001, each of which is incorporated herein by reference in its entirety), PVA (Chih-Ta Lee, Po-Han Kung and Yu-Der Lee, Carbohydrate Polymers, 61, 348, 2005, which is incorporated herein by reference in its entirety), collagen (Lee C R, Grodzinsky A J, Spector M., Biomaterials 22, 3145, 2001, which is incorporated herein by reference in its entirety), alginate (Bouhadir K H, Lee K Y, Alsberg E, Damm K L, Anderson K W, Mooney D J. Biotech Prog 17, 945, 2001; Smidsrd O, Skjak-Braek G., Trends Biotech, 8, 71, 1990, each of which is incorporated herein by reference in its entirety).

In some embodiments, the NK cells such as NKG2C^{pos} cells or a subset thereof can be present in the composition in an effective amount. In some embodiments, the composition contains an effective amount of g-NK cells, such as FcRγ^{neg} cells or cells having a g-NK surrogate marker profile thereof. An effective amount of cells can vary depending on the patient, as well as the type, severity and extent of disease. Thus, a physician can determine what an effective amount is after considering the health of the subject, the extent and severity of disease, and other variables.

In certain embodiments, the number of such cells in the composition is a therapeutically effective amount. In some embodiments, the amount is an amount that reduces the severity, the duration and/or the symptoms associated with cancer, viral infection, microbial infection, or septic shock in an animal. In some embodiments, a therapeutically effective amount is a dose of cells that results in a reduction of the growth or spread of cancer by at least 2.5%, at least 5%, at least 10%, at least 15%, at least 25%, at least 35%, at least 45%, at least 50%, at least 75%, at least 85%, by at least 90%, at least 95%, or at least 99% in a patient or an animal administered a composition described herein relative to the growth or spread of cancer in a patient (or an animal) or a group of patients (or animals) not administered the composition. In some embodiments, a therapeutically effective amount is an amount to result in cytotoxic activity resulting in activity to inhibit or reduce the growth of cancer, viral and microbial cells.

In some embodiments, the composition comprises an amount of NKG2C^{pos} cells or a subset thereof that is from at or about 10⁵ and at or about 10¹²NKG2C^{pos} cells or a subset thereof or from at or about 10⁵ to at or about 10⁸ NKG2C^{pos} cells or a subset thereof, or from at or about 10⁶ and at or about 10¹² NKG2C^{pos} cells or a subset thereof, or from at or about 10⁸ and at or about 10¹¹ NKG2C^{pos} cells or a subset thereof, or from at or about 10⁹ and at or about 10¹⁰ NKG2C^{pos} cells or a subset thereof. In some embodiments, the composition comprises greater than or greater than at or about 10⁵ NKG2C^{pos} cells or a subset thereof, at or about 10⁶ NKG2C^{pos} cells or a subset thereof, at or about 10⁷ NKG2C^{pos} cells or a subset thereof, at or about 10⁸ NKG2C^{pos} cells or a subset thereof, at or about 10⁹ NKG2C^{pos} cells or a subset thereof, at or about 10¹⁰ NKG2C^{pos} cells or a subset thereof, at or about 10¹¹ NKG2C^{pos} cells or a subset thereof, or at or about 10¹² NKG2C^{pos} cells or a subset thereof. In some embodiments, such an amount can be administered to a subject having a disease or condition, such as to a cancer patient.

In some embodiments, the composition comprises an amount of g-NK cells that is from at or about 10⁵ and at or about 10¹² g-NK cells, or from at or about 10⁵ to at or about 10⁸ g-NK cells, or from at or about 10⁶ and at or about 10¹² g-NK cells, or from at or about 10⁸ and at or about 10¹¹ g-NK cells, or from at or about 10⁹ and at or about 10¹⁰ g-NK cells. In some embodiments, the composition comprises greater than or greater than at or about 10⁵ g-NK cells, at or about 10⁶ g-NK cells, at or about 10⁷ g-NK cells, at or about 10⁸ g-NK cells, at or about 10⁹ g-NK cells, at or about 10¹⁰ g-NK cells, at or about 10¹¹ g-NK cells, or at or about 10¹² g-NK cells. In some embodiments, such an amount can be administered to a subject having a disease or condition, such as to a cancer patient.

In some embodiments, the volume of the composition is at least or at least about 10 mL, 50 mL, 100 mL, 200 mL, 300 mL, 400 mL or 500 mL, such as is from or from about 10 mL to 500 mL, 10 mL to 200 mL, 10 mL to 100 mL, 10 mL to 50 mL, 50 mL to 500 mL, 50 mL to 200 mL, 50 mL to 100 mL, 100 mL to 500 mL, 100 mL to 200 mL or 200 mL to 500 mL, each inclusive. In some embodiments, the composition has a cell density of at least or at least about 1×10⁵ cells/mL, 5×10⁵ cells/mL, 1×10⁶ cells/mL, 5×10⁶ cells/mL, 1×10⁷ cells/mL, 5×10⁷ cells/mL or 1×10⁸ cells/mL. In some embodiments, the cell density of the composition is between or between about 1×10⁵ cells/mL to 1×10⁸ cells/mL, 1×10⁵ cells/mL to 1×10⁷ cells/mL, 1×10⁵ cells/mL to 1×10⁶ cells/mL, 1×10⁶ cells/mL to 1×10⁷ cells/mL, 1×10⁶ cells/mL to 1×10⁸ cells/mL, 1×10⁶ cells/mL to 1×10⁷ cells/mL or 1×10⁷ cells/mL to 1×10⁸ cells/mL, each inclusive.

In some embodiments, the composition, including pharmaceutical composition, is sterile. In some embodiments, isolation, enrichment, or culturing of the cells is carried out in a closed or sterile environment, for example and for instance in a sterile culture bag, to minimize error, user handling and/or contamination. In some embodiments, sterility may be readily accomplished, e.g., by filtration through sterile filtration membranes. In some embodiments, culturing is carried out using a gas permeable culture vessel. In some embodiments, culturing is carried out using a bioreactor.

Also provided herein are compositions that are suitable for cryopreserving the provided NK cells. In some embodiments, the NK cells are cryopreserved in a serum-free cryopreservation medium. In some embodiments, the composition comprises a cryoprotectant. In some embodiments, the cryoprotectant is or comprises DMSO and/or s glycerol. In some embodiments, the cryopreservation medium is between at or about 5% and at or about 10% DMSO (v/v). In some embodiments, the cryopreservation medium is at or about 5% DMSO (v/v). In some embodiments, the cryopreservation medium is at or about 6% DMSO (v/v). In some embodiments, the cryopreservation medium is at or about 7% DMSO (v/v). In some embodiments, the cryopreservation medium is at or about 8% DMSO (v/v). In some embodiments, the cryopreservation medium is at or about 9% DMSO (v/v). In some embodiments, the cryopreservation medium is at or about 10% DMSO (v/v). In some embodiments, the cryopreservation medium contains a commercially available cryopreservation solution (CryoStor™ CS10). CryoStor™ CS10 is a cryopreservation medium containing 10% dimethyl sulfoxide (DMSO). In some embodiments, compositions formulated for cryopreservation can be stored at low temperatures, such as ultra low temperatures, for example, storage with temperature ranges from −40° C. to −150° C., such as or about 80° C.±6.0° C.

In some embodiments, the compositions can be preserved at ultra low temperature before the administration to a patient. In some aspects, NK cell subsets, such as g-NK cells, can be isolated, processed and expanded, such as in accord with the provided methods, and then stored at ultra-low temperature prior to administration to a subject.

A typical method for the preservation at ultra low temperature in small scale is described, for example, in U.S. Pat. No. 6,0168,991. For small-scale, cells can be preserved at ultra low temperature by low density suspension (e.g., at a concentration of about 200×106/ml) in 5% human albumin serum (HAS) which is previously cooled. An equivalent amount of 20% DMSO can be added into the HAS solution. Aliquots of the mixture can be placed into vials and frozen overnight inside an ultra low temperature chamber at about −80° C.

In some embodiments, the cryopreserved NK cells are prepared for administration by thawing. In some cases, the NK cells can be administered to a subject immediately after thawing. In such an embodiment, the composition is ready-to-use without any further processing. In other cases, the NK cells are further processed after thawing, such as by resuspension with a pharmaceutically acceptable carrier, incubation with an activating or stimulating agent, or are activated washed and resuspended in a pharmaceutically acceptable buffer prior to administration to a subject.

### V. METHODS FOR EXPANDING NATURAL KILLER CELL SUBSETS

Also disclosed herein is a method of producing a composition comprising a population or plurality of genetically engineered g-NK cell comprising a heterologous nucleic acid(s) encoding an antigen receptor (e.g. CAR). Also disclosed herein is a method of producing a composition comprising a population or plurality of genetically engineered g-NK cell comprising a heterologous nucleic acid(s) encoding an immunomodulator (e.g. cytokine, such as secretable or soluble cytokine or membrane-bound cytokine). Also disclosed herein is a method of producing a composition comprising a population or plurality of genetically engineered g-NK cell comprising a heterologous nucleic acid(s) encoding an antigen receptor (e.g. CAR) and an immunomodulator (e.g. cytokine, such as secretable or soluble cytokine or membrane-bound cytokine). In some embodiments, the methods comprise introducing into a g-NK cell, or a composition or population of cells enriched in or expanded for g-NK cells, a nucleic acid encoding a CAR.

Also disclosed herein is a method of producing a composition comprising a population or plurality of genetically engineered g-NK cell comprising a heterologous nucleic acid(s) encoding an immunomodulator (e.g. cytokine, such as secretable or soluble cytokine or membrane-bound cytokine). In some embodiments, the methods comprise introducing into a g-NK cell, or a composition or population of cells enriched in or expanded for g-NK cells, a nucleic acid encoding a an immunomodulator (e.g. cytokine, such as secretable or soluble cytokine or membrane-bound cytokine.

Also disclosed herein is a method of producing a composition comprising a population or plurality of genetically engineered g-NK cell comprising heterologous nucleic acid(s) encoding an antigen receptor (e.g. CAR) and an immunomodulator (e.g. cytokine, such as secretable or soluble cytokine or membrane-bound cytokine). In some embodiments, the methods comprise (a) introducing into a g-NK cell, or a composition or population of cells enriched in or expanded for g-NK cells, a nucleic acid encoding a CAR, and (b) introducing into the g-NK cell, or the composition or population of cells enriched in or expanded for g-NK cells, a nucleic acid encoding an immunomodulator (e.g. cytokine, such as secretable or soluble cytokine or membrane-bound cytokine, wherein steps (a) and (b) are carried out simultaneously or sequentially in any order.

In some embodiments, one or more steps of gene editing also can be carried out to produce cells in which a gene or genes has been edited, such as knocked-out, in the genome of the engineered cells. In some embodiments, the methods for gene editing, such as by introducing an RNA-guided nuclease, e.g. RNP complex, into the g-NK cell, or a composition or population of cells enriched in or expanded for g-NK cells, can be carried out simultaneously or sequentially with the steps of introducing a heterologous nucleic acid encoding an antigen receptor (e.g. CAR), a heterologous nucleic acid encoding an immunomodulator (e.g. cytokine, such as secretable or soluble cytokine or membrane-bound cytokine), or heterologous nucleic acids encoding an antigen receptor (e.g. CAR) and an immunomodulator (e.g. cytokine, such as secretable or soluble cytokine or membrane-bound cytokine), in any order.

In some embodiments, the steps of engineering the cells, can be carried out in connection with a method for enriching and expanding g-NK cells from a biological sample from a subject. Methods for enriching or expanding g-NK cells may include method as described in PCT Publication No. WO2020/107002 or PCT Appl. No. PCT/US2021/028504. Exemplary methods for enriching for g-NK cells, and preferentially expanding such cells, are described in further detail below.

In some aspects, the nucleic acid encoding the CAR can be introduced into the g-NK cell for stable integration into the genome or for transient expression. In some aspects, the nucleic acid encoding the immunomodulator can be introduced into the g-NK cell for stable integration into the genome or for transient expression. In some aspects, the nucleic acids encoding the CAR and the immunomodulator can be introduced into the g-NK cell for stable integration into the genome or for transient expression. When the nucleic acid is introduced into the g-NK cell for stable integration, it can be introduced prior to culturing the population of engineered NK cells, such that the nucleic acid is stably integrated and will be propagated in the engineered NK cell progeny. In some embodiments, the nucleic acid encoding the CAR is stably integrated into the genome. In some embodiments, the nucleic acid encoding the immunomodulator is stably integrated into the genome. In some embodiments, the nucleic acids encoding the CAR and the immunomodulator is stably integrated into the genome. In some embodiments, the nucleic acid is introduced to the g-NK cell via a viral vector. In some embodiments, the viral vector is a lentiviral vector.

In some embodiments, the steps of engineering the cells, such as for stable integration of the heterologous agents into the genome of the NK cell, is carried out prior to culturing or incubating enriched g-NK cells under conditions for their further expansion, In some embodiments, NK cells are isolated from a biological sample as described in Section V. A below, and then are introduced with a heterologous nucleic acid(s) encoding an antigen receptor (e.g. CAR) prior to expanding the cells using methods as described in Section V. B. In some embodiments, NK cells are isolated from a biological sample as described in Section V. A below, and then are introduced with a heterologous nucleic acid(s) encoding an immunomodulator (e.g. cytokine, such as secretable or soluble cytokine or membrane-bound cytokine) prior to expanding the cells using methods as described in Section V. B. In some embodiments, NK cells are isolated from a biological sample as described in Section V. A below, and then are introduced with a heterologous nucleic acid(s) encoding an antigen receptor (e.g. CAR) and an immunomodulator (e.g. cytokine, such as secretable or soluble cytokine or membrane-bound cytokine) prior to expanding the cells using methods as described in Section V. B. In some embodiments, the cells are further engineered by gene editing methods simultaneously or sequentially with introducing the nucleic acid encoding the CAR. In some embodiments, the cells are further engineered by gene editing methods simultaneously or sequentially with introducing the nucleic acid encoding the immunomodulator (e.g. cytokine, such as secretable or soluble cytokine or membrane-bound cytokine). In some embodiments, the cells are further engineered by gene editing methods simultaneously or sequentially with introducing the nucleic acids encoding the CAR and the immunomodulator (e.g. cytokine, such as secretable or soluble cytokine or membrane-bound cytokine). In some embodiments, the cells are engineered by gene editing prior to carrying out the expansion methods described in Section V. B.

In some embodiments, the steps of engineering the cells, such as for stable integration of the heterologous agents into the genome of the NK cells, is carried out during the culturing or incubating enriched g-NK cells under conditions for their further expansion. For instance, NK cells are isolated from a biological sample as described in Section V. A below, and then are subjected to a first period of expansion in accord with the methods described in Section V. B. The first period of expansion is a portion of the total expansion period as described in Section V.B, in which the remaining portion of the expansion period is achieved by carrying out a second period of expansion. For instance, the first expansion period is for at or about 1 day, 2 days, 3 days, 4 days, 5 days, 6 days, 7 days, 8 days, 9 days or 10 days. In some embodiments, after the first period of expansion, the cells are collected and then introduced with a heterologous nucleic acid(s) encoding an antigen receptor (e.g. CAR) prior to further expanding the cells in a second expansion period using methods as described in Section V. B. In some embodiments, after the first period of expansion, the cells are collected and then introduced with a heterologous nucleic acid(s) encoding an immunomodulator (e.g. cytokine, such as secretable or soluble cytokine or membrane-bound cytokine) prior to further expanding the cells in a second expansion period using methods as described in Section V. B. In some embodiments, after the first period of expansion, the cells are collected and then introduced with a heterologous nucleic acid(s) encoding an antigen receptor (e.g. CAR) and an immunomodulator (e.g. cytokine, such as secretable or soluble cytokine or membrane-bound cytokine) prior to further expanding the cells in a second expansion period using methods as described in Section V. B. The second period of expansion is a portion of the total expansion period as described in Section V. B, such as until a threshold number of cells enriched in g-NK cells are expanded. For instance, the second expansion period is for at or about 1 day, 2 days, 3 days, 4 days, 5 days, 6 days, 7 days, 8 days, 9 days or 10 days. In some embodiments, the cells are further engineered by gene editing methods simultaneously or sequentially with introducing the nucleic acid encoding the CAR In some embodiments, the cells are further engineered by gene editing methods simultaneously or sequentially with introducing the nucleic acid encoding the immunomodulator (e.g. cytokine, such as secretable or soluble cytokine or membrane-bound cytokine). In some embodiments, the cells are further engineered by gene editing methods simultaneously or sequentially with introducing the nucleic acids encoding the CAR and the immunomodulator (e.g. cytokine, such as secretable or soluble cytokine or membrane-bound cytokine). In some embodiments, the cells are engineered by gene editing after the isolating or selecting cells from the biological sample to enrich for g-NK cells as described in Section V. A, and prior to carrying out the first expansion.

In other aspects, the nucleic acid encoding the CAR can be introduced into the g-NK cell for transient expression. In other aspects, the nucleic acid encoding the immunomodulator can be introduced into the g-NK cell for transient expression. In other aspects, the nucleic acids encoding the CAR and the immunomodulator can be introduced into the g-NK cell for transient expression. When the nucleic acid is introduced into the g-NK cell for transient expression, it can be introduced after culturing the population of engineered NK cells, as a transiently expressed nucleic acid may not persist for sufficiently long periods of time or be propagated sufficiently into all cells of the cultured population. In some embodiments, the nucleic acid encoding the CAR is transiently expressed in the engineered NK cell. In some embodiments, the nucleic acid encoding the immunomodulator (e.g. cytokine, such as secretable or soluble cytokine or membrane-bound cytokine) is transiently expressed in the engineered NK cell. In some embodiments, the nucleic acids encoding the CAR and the immunomodulator (e.g. cytokine, such as secretable or soluble cytokine or membrane-bound cytokine) is transiently expressed in the engineered NK cell. In some embodiments, the nucleic acid is introduced via nanoparticle delivery. In some embodiments, the nucleic acid is introduced via electroporation.

Thus, in some methods of expanding g-NK cells, the population of enriched NK cells should be cultured under conditions for expansion prior to introducing into the NK cells of the expanded population the nucleic acid encoding the CAR. In some methods of expanding g-NK cells, the population of enriched NK cells should be cultured under conditions for expansion prior to introducing into the NK cells of the expanded population the nucleic acid encoding the immunomodulator (e.g. cytokine, such as secretable or soluble cytokine or membrane-bound cytokine). In some methods of expanding g-NK cells, the population of enriched NK cells should be cultured under conditions for expansion prior to introducing into the NK cells of the expanded population the nucleic acids encoding the CAR and the immunomodulator (e.g. cytokine, such as secretable or soluble cytokine or membrane-bound cytokine). This is particularly applicable in situations where the CAR or the immunomodulator is expressed transiently (e.g., via mRNA).

Exemplary methods for expansion of NK cells enriched in g-NK cells is described in Sections V. A and B below. In some embodiments, the method of expanding g-NK cells comprises (a) obtaining a population of primary human cells enriched for Natural Killer (NK) cells, wherein the population enriched for NK cells is selected from a biological sample from a human subject; (b) culturing the population of enriched NK cells in culture medium with (i) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (ii) an effective amount of two or more recombinant cytokines for expansion of the NK cells, wherein at least one recombinant cytokine is interleukin (IL)-2 and at least one recombinant cytokine is IL-21, thereby producing an expanded population of NK cells; (c) introducing into NK cells of the expanded population of NK cells nucleic acid encoding a CAR, wherein the method produces an expanded population of engineered NK cells that are enriched in g-NK cells engineered with a CAR. In some embodiments, the method of expanding g-NK cells comprises (a) obtaining a population of primary human cells enriched for Natural Killer (NK) cells, wherein the population enriched for NK cells is selected from a biological sample from a human subject; (b) culturing the population of enriched NK cells in culture medium with (i) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (ii) an effective amount of two or more recombinant cytokines for expansion of the NK cells, wherein at least one recombinant cytokine is interleukin (IL)-2 and at least one recombinant cytokine is IL-21, thereby producing an expanded population of NK cells; (c) introducing into NK cells of the expanded population of NK cells nucleic acid encoding an immunomodulator (e.g. cytokine, such as secretable or soluble cytokine or membrane-bound cytokine), wherein the method produces an expanded population of engineered NK cells that are enriched in g-NK cells engineered with an immunomodulator (e.g. cytokine, such as secretable or soluble cytokine or membrane-bound cytokine). In some embodiments, the method of expanding g-NK cells comprises (a) obtaining a population of primary human cells enriched for Natural Killer (NK) cells, wherein the population enriched for NK cells is selected from a biological sample from a human subject; (b) culturing the population of enriched NK cells in culture medium with (i) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (ii) an effective amount of two or more recombinant cytokines for expansion of the NK cells, wherein at least one recombinant cytokine is interleukin (IL)-2 and at least one recombinant cytokine is IL-21, thereby producing an expanded population of NK cells; (c) introducing into NK cells of the expanded population of NK cells (i) nucleic acid encoding a CAR, and (ii) a nucleic acid encoding an immunomodulator (e.g. cytokine, such as secretable or soluble cytokine or membrane-bound cytokine), wherein steps (i) and (ii) are carried out simultaneously or sequentially in any order, wherein the method produces an expanded population of engineered NK cells that are enriched in g-NK cells engineered with a CAR and an immunomodulator (e.g. cytokine, such as secretable or soluble cytokine or membrane-bound cytokine).

In some embodiments, the steps of engineering the cells, such as for transient expression of the heterologous agents into the genome of the NK cell, is carried out after culturing or incubating enriched g-NK cells under conditions for their further expansion. In some embodiments, NK cells are isolated from a biological sample as described in Section V. A below, expanded using methods as described in Section V. B, and then are introduced with a heterologous nucleic acid(s) encoding an antigen receptor (e.g. CAR). In some embodiments, NK cells are isolated from a biological sample as described in Section V. A below, expanded using methods as described in Section V. B, and then are introduced with a heterologous nucleic acid(s) encoding an immunomodulator (e.g. cytokine, such as secretable or soluble cytokine or membrane-bound cytokine). In some embodiments, NK cells are isolated from a biological sample as described in Section V. A below, expanded using methods as described in Section V. B, and then are introduced with heterologous nucleic acids encoding an antigen receptor (e.g. CAR) and an immunomodulator (e.g. cytokine, such as secretable or soluble cytokine or membrane-bound cytokine). In some embodiments, the cells are further engineered by gene editing methods simultaneously or sequentially with introducing the nucleic acid encoding the CAR and/or the immunomodulator (e.g. a cytokine, such as a secretable or soluble cytokine or membrane-bound cytokine). In some embodiments, the cells are engineered by gene editing prior to carrying out the expansion methods described in Section V. B.

A. Methods of Selecting Cells from a Biological Sample for Enriching g-NK Cells

In some embodiments, the g-NK cell compositions, including compositions containing engineered g-NK cells, are produced by methods that include methods for enriching g-NK cells by their expansion ex vivo from a subset of NK cells from a biological sample from a human subject. In some embodiments, the methods for expanding and producing a g-NK cell composition can include expanding a subset of cells that are FcRγ-deficient NK cells (g-NK) from a biological sample from a human subject. In some embodiments, the methods can include expanding a subset of NK cells that are NKG2C^{pos} from a biological sample from a human subject. In some embodiments, the methods can include expanding a subset of NK cells that are NKG2A^{neg} from a biological sample from a human subject. In some embodiments, the method includes isolating a population of cells enriched for natural killer (NK) cells from a biological sample from a human subject and culturing the cells under conditions in which preferential growth and/or expansion of the g-NK cell subject and/or an NK cell subset that overlaps or shares extracellular surface markers with the g-NK cell subset. For example, the NK cells may be cultured using feeder cells, or in the presence of cytokines to enhance the growth and/or expansion of g-NK cell subject and/or an NK cell subset that overlaps or shares extracellular surface markers with the g-NK cell subset. In some aspects, the provided methods also can expand other subsets of NK cells, such as any NK cell that is NKG2C^{pos} and/or NKG2A^{neg}.

In some embodiments, the sample, e.g. biological sample, is one containing a plurality of cell populations that includes an NK cell population. In some embodiments, the biological sample is or comprises blood cells, e.g. peripheral blood mononuclear cells. In some aspects, the biological sample is a whole blood sample, an apheresis product or a leukapheresis product. In some embodiments, the sample is a sample of peripheral blood mononuclear cells (PBMCs). Thus, in some embodiments of the provided methods, a population of peripheral blood mononuclear cells (PBMCs) can be obtained. The sample containing a plurality of cell populations that includes an NK cell population can be used as the cells for enriching or selecting an NK cell subset for expansion in accord with the provided methods.

In some embodiments, the biological sample is from a subject that is a healthy subject. In some embodiments, the biological sample is from a subject that has a disease of conditions, e.g. a cancer.

In some embodiments, the cells are isolated or selected from a sample, such as a biological sample, e.g., one obtained from or derived from a subject, such as one having a particular disease or condition or in need of a cell therapy or to which cell therapy will be administered. In some aspects, the subject is a human, such as a subject who is a patient in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom. In some aspects, the sample is blood or a blood-derived sample, or is or is derived from an apheresis or leukapheresis product.

In some examples, cells from the circulating blood of a subject are obtained. The samples, in some aspects, contain lymphocytes, including NK cells, T cells, monocytes, granulocytes, B cells, other nucleated white blood cells, red blood cells, and/or platelets, and in some aspects contains cells other than red blood cells and platelets. In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient, such as by using a Histopaque® density centrifugation.

In some embodiments, the biological sample is from an enriched leukapheresis product collected from normal peripheral blood. In some embodiments, the enriched leukapheresis product can contain fresh cells. In some embodiments, the enriched leukapheresis product is a cryopreserved sample that is thawed for use in the provided methods.

In some embodiments, the source of biological cells contains from at or about 5×10, to at or about 5×10⁸ NK cells or a g-NK cell subset or an NK cell subset that is associated with or includes a surrogate marker for g-NK cells. In some embodiments, the number of NK cells, or a g-NK cell subset or an NK cell subset that is associated with or includes a surrogate marker for g-NK cells, in the biological sample is from at or about 5×10⁵ to at or about 1×10⁸, from at or about 5×10⁵ to at or about 5×10⁷, from at or about 5×10⁵ to at or about 1×10⁷, from at or about 5×10⁵ to at or about 5×10⁶, from at or about 5×10⁵ to at or about 1×10⁶, from at or about 1×10⁶ to at or about 1×10⁸, from at or about 1×10⁶ to at or about 5×10⁷, from at or about 1×10⁶ to at or about 1×10⁷, from at or about 1×10⁶ to at or about 5×10⁶, from at or about 5×10⁶ to at or about 1×10⁸, from at or about 5×10⁶ to at or about 5×10⁷, from at or about 5×10⁶ to at or about 1×10⁷, from at or about 1×10⁷ to at or about 1×10⁸, from at or about 1×10⁷ to at or about 5×10⁷, or from at or about 5×10⁷ to at or about 1×10⁸.

In some embodiments, the percentage of g-NK cells, or of an NK cell subset that is associated with or includes a surrogate marker for g-NK cells, among NK cells in the biological sample is greater than at or about 3%. In some embodiments, the percentage of g-NK cells, or of an NK cell subset that is associated with or includes a surrogate marker for g-NK cells, among NK cells in the biological sample is greater than at or about 5%. In some embodiments, the percentage of g-NK cells, or of an NK cell subset that is associated with or includes a surrogate marker for g-NK cells, among NK cells in the biological sample is greater than at or about 10%. In some embodiments, the percentage of g-NK cells, or of an NK cell subset that is associated with or includes a surrogate marker for g-NK cells, among NK cells in the biological sample is greater than at or about 12%. In some embodiments, the percentage of g-NK cells, or of an NK cell subset that is associated with or includes a surrogate marker for g-NK cells, among NK cells in the biological sample is greater than at or about 14%. In some embodiments, the percentage of g-NK cells, or of an NK cell subset that is associated with or includes a surrogate marker for g-NK cells, among NK cells in the biological sample is greater than at or about 16%. In some embodiments, the percentage of g-NK cells, or of an NK cell subset that is associated with or includes a surrogate marker for g-NK cells, among NK cells in the biological sample is greater than at or about 18%. In some embodiments, the percentage of g-NK cells, or of an NK cell subset that is associated with or includes a surrogate marker for g-NK cells, among NK cells in the biological sample is greater than at or about 20%. In some embodiments, the percentage of g-NK cells, or of an NK cell subset that is associated with or includes a surrogate marker for g-NK cells, among NK cells in the biological sample is greater than at or about 22%. In some embodiments, the percentage of g-NK cells, or of an NK cell subset that is associated with or includes a surrogate marker for g-NK cells, among NK cells in the biological sample is greater than at or about 24%. In some embodiments, the percentage of g-NK cells, or of an NK cell subset that is associated with or includes a surrogate marker for g-NK cells, among NK cells in the biological sample is greater than at or about 26%. In some embodiments, the percentage of g-NK cells, or of an NK cell subset that is associated with or includes a surrogate marker for g-NK cells, among NK cells in the biological sample is greater than at or about 28%. In some embodiments, the percentage of g-NK cells, or of an NK cell subset that is associated with or includes a surrogate marker for g-NK cells, among NK cells in the biological sample is greater than at or about 30%.

In some embodiments, a subject is selected if the percentage of g-NK cells, or of an NK cell subset that is associated with or includes a surrogate marker for g-NK cells, among NK cells in the biological sample is greater than at or about 3%. In some embodiments, a subject is selected if the percentage of g-NK cells, or of an NK cell subset that is associated with or includes a surrogate marker for g-NK cells, among NK cells in the biological sample is greater than at or about 5%. In some embodiments, a subject is selected if the percentage of g-NK cells, or of an NK cell subset that is associated with or includes a surrogate marker for g-NK cells, among NK cells in the biological sample is greater than at or about 10%. In some embodiments, a subject is selected if the percentage of g-NK cells, or of an NK cell subset that is associated with or includes a surrogate marker for g-NK cells, among NK cells in the biological sample is greater than at or about 12%. In some embodiments, a subject is selected if the percentage of g-NK cells, or of an NK cell subset that is associated with or includes a surrogate marker for g-NK cells, among NK cells in the biological sample is greater than at or about 14%. In some embodiments, a subject is selected if the percentage of g-NK cells, or of an NK cell subset that is associated with or includes a surrogate marker for g-NK cells, among NK cells in the biological sample is greater than at or about 16%. In some embodiments, a subject is selected if the percentage of g-NK cells, or of an NK cell subset that is associated with or includes a surrogate marker for g-NK cells, among NK cells in the biological sample is greater than at or about 18%. In some embodiments, a subject is selected if the percentage of g-NK cells, or of an NK cell subset that is associated with or includes a surrogate marker for g-NK cells, among NK cells in the biological sample is greater than at or about 20%. In some embodiments, a subject is selected if the percentage of g-NK cells, or of an NK cell subset that is associated with or includes a surrogate marker for g-NK cells, among NK cells in the biological sample is greater than at or about 22%. In some embodiments, a subject is selected if the percentage of g-NK cells, or of an NK cell subset that is associated with or includes a surrogate marker for g-NK cells, among NK cells in the biological sample is greater than at or about 24%. In some embodiments, a subject is selected if the percentage of g-NK cells, or of an NK cell subset that is associated with or includes a surrogate marker for g-NK cells, among NK cells in the biological sample is greater than at or about 26%. In some embodiments, a subject is selected if the percentage of g-NK cells, or of an NK cell subset that is associated with or includes a surrogate marker for g-NK cells, among NK cells in the biological sample is greater than at or about 28%. In some embodiments, a subject is selected if the percentage of g-NK cells, or of an NK cell subset that is associated with or includes a surrogate marker for g-NK cells, among NK cells in the biological sample is greater than at or about 30%.

In some embodiments, the biological sample is from a subject that is CMV seropositive. CMV infection can result in phenotypic and functional differentiation of NK cells, including development of high fractions of NK cells expressing NKG2C that exhibit enhanced antiviral activity. CMV-associated NK cells expressing NKG2C display altered DNA methylation patterns and reduced expression of signaling molecules, such as FcRγ (Schlums et al., Immunity (2015) 42:443-56). These NK cells are linked to more potent antibody-dependent activation, expansion, and function relative to conventional NK-cell subsets. In some cases, the biological sample can be from a subject that is CMV seronegative as NK cells with reduced expression of FcRγ can also be detected in CMV seronegative individuals, albeit generally at lower levels. In some cases, the biological sample can be from CMV seropositive individuals.

In some embodiments, a subject is selected based on the percentage of NK cells in a peripheral blood sample that are positive for NKG2C. In some embodiments, the subject is selected if at least at or about 20% of NK cells in the peripheral blood sample are positive for NKG2C. In some embodiments, the subject is selected if at least at or about 25% of NK cells in the peripheral blood sample are positive for NKG2C. In some embodiments, the subject is selected if at least at or about 30% of NK cells in the peripheral blood sample are positive for NKG2C. In some embodiments, the subject is selected if at least at or about 35% of NK cells in the peripheral blood sample are positive for NKG2C. In some embodiments, the subject is selected if at least at or about 40% of NK cells in the peripheral blood sample are positive for NKG2C. In some embodiments, the subject is selected if at least at or about 45% of NK cells in the peripheral blood sample are positive for NKG2C. In some embodiments, the subject is selected if at least at or about 50% of NK cells in the peripheral blood sample are positive for NKG2C. In some embodiments, the subject is selected if at least at or about 55% of NK cells in the peripheral blood sample are positive for NKG2C. In some embodiments, the subject is selected if at least at or about 60% of NK cells in the peripheral blood sample are positive for NKG2C.

In some embodiments, a subject is selected based on the percentage of NK cells in a peripheral blood sample that are negative or low for NKG2A. In some embodiments, a subject is selected if at least at or about 70% of NK cells in the peripheral blood sample are negative or low for NKG2A. In some embodiments, a subject is selected if at least at or about 75% of NK cells in the peripheral blood sample are negative or low for NKG2A. In some embodiments, a subject is selected if at least at or about 80% of NK cells in the peripheral blood sample are negative or low for NKG2A. In some embodiments, a subject is selected if at least at or about 85% of NK cells in the peripheral blood sample are negative or low for NKG2A. In some embodiments, a subject is selected if at least at or about 90% of NK cells in the peripheral blood sample are negative or low for NKG2A.

In some embodiments, a subject is selected based on both the percentage of NK cells in a peripheral blood sample that are positive for NKG2C and the percentage of NK cells in the peripheral blood sample that are negative or low for NKG2A. In some embodiments, the subject is selected if at least at or about 20% of NK cells in the peripheral blood sample are positive for NKG2C and at least at or about 70% of NK cells in the peripheral blood sample are negative or low for NKG2A. In some embodiments, the subject is selected if at least at or about 30% of NK cells in the peripheral blood sample are positive for NKG2C and at least at or about 75% of NK cells in the peripheral blood sample are negative or low for NKG2A. In some embodiments, the subject is selected if at least at or about 40% of NK cells in the peripheral blood sample are positive for NKG2C and at least at or about 80% of NK cells in the peripheral blood sample are negative or low for NKG2A. In some embodiments, the subject is selected if at least at or about 50% of NK cells in the peripheral blood sample are positive for NKG2C and at least at or about 85% of NK cells in the peripheral blood sample are negative or low for NKG2A. In some embodiments, the subject is selected if at least at or about 60% of NK cells in the peripheral blood sample are positive for NKG2C and at least at or about 90% of NK cells in the peripheral blood sample are negative or low for NKG2A. In some embodiments, the subject is selected if at least at or about 60% of NK cells in the peripheral blood sample are positive for NKG2C and at least at or about 95% of NK cells in the peripheral blood sample are negative or low for NKG2A.

In some embodiments, a subject is selected for expansion of cells in accord with the provided methods if the subject is CMV seropositive, and if among NK cells in a peripheral blood sample from the subject, the percentage of g-NK cells is greater than at or about 30%, the percentage of NKG2C^{pos} cells is greater than at or about 20%, and the percentage of NKG2A^{neg} cells is greater than at or about 70%.

In some embodiments, NK cells from the subject bear a single nucleotide polymorphism (SNP rs396991) in the CD16 gene, nucleotide 526 [thymidine (T)→guanine (G)] resulting in an amino acid (aa) substitution of valine (V) for phenylalanine (F) at position 158 in the mature (processed) form of the protein (F158V). In some embodiments, NK cells bear the CD16 158V polymorphism in both alleles (called 158V/V herein). In some embodiments, NK cells bear the CD16 158V polymorphism in a single allele (called 158V/F herein). It is understood that reference to a 158V+ genotype herein refers to both the 158V/V genotype and the 158V/F genotype. It has been found that the CD16 F158V polymorphism is associated with substantially higher affinity for IgG1 antibodies and have the ability to mount more robust NK cell-mediated ADCC responses (Mellor et al. (2013) Journal of Hematology & Oncology, 6:1; Musolino et al. (2008) Journal of Clinical Oncology, 26:1789-1796 and Hatjiharissi et al. (2007) Blood, 110:2561-2564). In some embodiments, antibody-directed targeting of CD16 158V+/g-NK cells leads to improved outcomes for patients due to the improved affinity, cytotoxic and/or cytokine-mediated effect functions of the CD16 158V+/g-NK cell subset.

In some embodiments, the provided methods include enriching or isolating NK cells or a subset thereof from a biological sample of a subject identified as having the CD16 158V+ NK cell genotype. In some embodiments, the method includes screening subjects for the presence of the CD16 158V+ NK cell genotype. In some embodiments, genomic DNA is extracted from a sample from a subject that is or includes NK cells, such as blood sample or bone marrow sample. In some embodiments, the sample is or comprises blood cells, e.g. peripheral blood mononuclear cells. In some embodiments, the sample is or comprises isolated NK cells. In some embodiments, the sample is a sample from a healthy donor subject. Any method for extracting DNA from the sample can be employed. For instance, nucleic acids can be readily isolated from a sample, e.g. cells, using standard techniques such as guanidium thiocyanate-phenol-chloroform extraction (Chomocyznski et al. (1987) Anal. Biochem. 162: 156). Commercially available kits also are readily available for extracting genomic DNA, such as the Wizard genomic DNA purification kit (Promega, Madison, WI).

Genotyping can be performed on any suitable sample. In any of the embodiments described herein, the genotyping reaction can be, for example, a pyrosequencing reaction, DNA sequencing reaction, MassARRAY MALDI-TOF, RFLP, allele-specific PCR, real-time allelic discrimination, or microarray. In some embodiments, a PCR-based technique, such as RT-PCR, of genomic DNA is carried out using allele-specific primers for the polymorphism. The PCR method for amplifying target nucleic acid sequences in a sample is well known in the art and has been described in, e.g., Innis et al. (eds.) PCR Protocols (Academic Press, N Y 1990); Taylor (1991) Polymerase chain reaction: basic principles and automation, in PCR A Practical Approach, McPherson et al. (eds.) IRL Press, Oxford; Saiki et al. (1986) Nature 324: 163; as well as in U.S. Pat. Nos. 4,683,195, 4,683,202 and 4,889,818, all incorporated herein by reference in their entireties.

Primers for detecting the 158V+ polymorphism are known or can be easily designed by a skilled artisan, See. e.g. International published PCT Appl. No. WO2012/061814; Kim et al. (2006) Blood, 108:2720-2725; Cartron et al. (2002) Blood, 99:754-758; Koene et al. (1997) Blood, 90:1109-1114; Hatijiharissi et al. (2007) Blood, 110:2561-2564; Somboonyosdech et al. (2012) Asian Biomedicine, 6:883-889). In some embodiments, PCR can be carried out using nested primers followed by allele-specific restriction enzyme digestion. In some embodiments, the first PCR primers comprise nucleic acid sequences 5′-ATA TTT ACA GAA TGG CAC AGG-3′ (SEQ ID NO:17) and 5′-GAC TTG GTA CCC AGG TTG AA-3′ (SEQ ID NO:18), while the second PCR primers are 5′-ATC AGA TTC GAT CCT ACT TCT GCA GGG GGC AT-3′ (SEQ ID NO:19) and 5′-ACG TGC TGA GCT TGA GTG ATG GTG ATG TTC AC-3′ (SEQ ID NO:20), which, in some cases, generates a 94-bp fragment depending on the nature of allele. In some embodiments, the primer pair comprises the nucleic acid sequences set forth in SEQ ID NO:21 (CCCAACTCAA CTTCCCAGTG TGAT) and SEQ ID NO:22 (GAAATCTACC TTTTCCTCTA ATAGGGCAAT). In some embodiments, the primer pair comprises the nucleic acid sequences set forth in SEQ ID NO:21 (CCCAACTCAA CTTCCCAGTG TGAT) and SEQ ID NO:23 (GAAATCTACC TTTTCCTCTA ATAGGGCAA). In some embodiments, the primer pair comprises the nucleic acid sequences set forth in SEQ ID NO:21 (CCCAACTCAA CTTCCCAGTG TGAT) and SEQ ID NO:24 (GAAATCTACC TTTTCCTCTA ATAGGGCA). In some embodiments, genotyping can be carried out by quantitative real-time RT-PCR following extraction of RNA using primer sequences as follows: CD16 sense set forth in SEQ ID NO:25 (5′-CCAAAAGCCACACTCAAAGAC-3′) and antisense set forth in SEQ ID NO:26 (5′-ACCCAGGTGGAAAGAATGATG-3′) and TaqMan probe set forth in SEQ ID NO:27 (5′-AACATCACCATCACTCAAGGTTTGG-3′).

To confirm the genotyping, allele specific amplification can be used with a set of V allele specific primers (e.g. forward primer set forth in SEQ ID NO:28, 5′-CTG AAG ACA CAT TTT TAC TCC CAAA-3′; and reverse primer set forth in SEQ ID NO:29, 5′-TCC AAA AGC CAC ACT CAA AGA C-3′) or a set of F allele specific primers (e.g., forward primer set forth in SEQ ID NO:30, 5′-CTG AAG ACA CAT TTT TAC TCC CAAC-3′; and reverse primer set forth in SEQ ID NO:29, 5′-TCC AAA AGC CAC ACT CAA AGA C-3′).

The genomic sequence for CD16a is available in the NCBI database at NG_009066.1. The gene ID for CD16A is 2214. Sequence information for CD16, including gene polymorphisms, is available at UniProt Acc. No. P08637. The sequence of CD16 (F158) is set forth in SEQ ID NO:31 (residue F158 is bold and underlined). In some embodiments, CD16 (F158) further comprises a signal peptide set forth as MWQLLLPTALLLLVSA (SEQ ID NO:32).

(SEQ ID NO: 31)
GMRTEDLPKAVVFLEPQWYRVLEKDSVTLKCQGAYSPEDNSTQWFHNES
LISSQASSYFIDAATVDDSGEYRCQTNLSTLSDPVQLEVHIGWLLLQAP
RWVFKEEDPIHLRCHSWKNTALHKVTYLONGKGRKYFHHNSDFYIPKAT
LKDSGSYFCRGLFGSKNVSSETVNITITQGLAVSTISSFFPPGYQVSFC
LVMVLLFAVDTGLYFSVKTNIRSSTRDWKDHKFKWRKDPQDK

The sequence of CD16 158V+ (polymorphism resulting in F158V) is known as VAR_003960 and has the sequence set forth in SEQ ID NO:33 (158V+ polymorphism is in bold and underline). In some embodiments, CD16 (158V+) further comprises a signal peptide set forth as MWQLLLPTALLLLVSA (SEQ ID NO:32).

(SEQ ID NO: 33)
GMRTEDLPKAVVFLEPQWYRVLEKDSVTLKCQGAYSPEDNSTQWFHNES
LISSQASSYFIDAATVDDSGEYRCQTNLSTLSDPVQLEVHIGWLLLQAP
RWVFKEEDPIHLRCHSWKNTALHKVTYLQNGKGRKYFHHNSDFYIPKAT
LKDSGSYFCRGLVGSKNVSSETVNITITQGLAVSTISSFFPPGYQVSFC
LVMVLLFAVDTGLYFSVKTNIRSSTRDWKDHKFKWRKDPQDK

In some embodiments, single nucleotide polymorphism (SNP) analysis is employed on genomic deoxyribonucleic acid (DNA) samples using allele-specific probes containing a fluorescent dye label (e.g. FAM or VIC) on the 5′ end and a minor groove binder (MGB) and nonfluorescent quencher (NFQ) on the 3′ end and an unlabeled PCR primers to detect a specific SNP targets. In some embodiments, the assay measures or detects the presence of an SNP by a change in fluorescence of the dyes associated with the probe. In such embodiments, probes hybridize to the target DNA between the two unlabeled primers and signal from the fluorescent dye on the 5′ end is quenched by the NFQ on its 3′ end by fluorescence resonance energy transfer (FRET). During PCR, Taq polymerase extends the unlabeled primers using the template as a guide and when the polymerase reaches the labeled probe, it cleaves the molecule separating the dye from the quencher. In some aspects, a qPCR instrument can detect fluorescence from the unquenched label. Exemplary reagents are commercially available SNP Assays, e.g. code C_25815666_10 for rs396991 (Applied Biosystems, Cat No. 4351379 for SNP genotyping of F158V in CD16).

In some embodiments, subjects heterozygous or homozygous for the CD16 158V (F158V) polymorphism are identified. In some embodiments, subjects homozygous for the CD16 158V (F158V) polymorphism are identified. In some embodiments, NK cells or an NK cell subset are isolated or enriched from a biological sample from a subject identified as being heterozygous or homozygous for the CD16 158V polymorphism. In some embodiments, NK cells or an NK cell subset are isolated or enriched from a biological sample from a subject identified as being homozygous for the CD16 158V polymorphism.

In some embodiments, the method includes enriching NK cells from the biological sample, such as from a population PBMCs isolated or obtained from the subject. In some embodiments, the population of cells enriched for NK cells is enriched by isolation or selection based on one or more natural killer cell-specific markers. It is within the level of a skilled artisan to choose particular markers or combinations of surface markers. In some embodiments, the surface marker(s) is any one or more of the from the following surface antigens CD11a, CD3, CD7, CD14, CD16, CD19, CD25, CD27, CD56, CD57, CD161, CD226, NKB1, CD62L; CD244, NKG2D, NKp30, NKp44, NKp46, NKG2A, NKG2C, KIR2DL 1 and/or KIR2DL3. In some embodiments, the surface marker(s) is any one or more of the from the following surface antigens CD11a, CD3, CD7, CD14, CD16, CD19, CD25, CD27, CD38, CD56, CD57, CD161, CD226, NKB1, CD62L; CD244, NKG2D, NKp30, NKp44, NKp46, NKG2A, NKG2C, SLAMF7 (CD319), KIR2DL 1 and/or KIR2DL3. In particular embodiments, the one or more surface antigen includes CD3 and one or more of the following surface antigens CD16, CD56 or CD57. In some embodiments, the one or more surface antigen is CD3 and CD57. In some embodiments, the one or more surface antigen is CD3, CD56 and CD16. In other embodiments, the one or more surface antigen is CD3, CD56 and CD38. In further embodiments, the one or more surface antigen is CD3, CD56, NKG2A and CD161. In some embodiments, the one or more surface antigen is CD3, CD57, and NKG2C. In some embodiments, the one or more surface antigen is CD3, CD57, and NKG2A. In some embodiments, the one or more surface antigen is CD3, CD57, NKG2C, and NKG2A. In some embodiments, the one or more surface antigen is CD3 and CD56. In some embodiments, the one or more surface antigen is CD3, CD56, and NKG2C. In some embodiments, the one or more surface antigen is CD3, CD56, and NKG2A. In some embodiments, the one or more surface antigen is CD3, CD56, NKG2C, and NKG2A. Reagents, including fluorochrome-conjugated antibodies, for detecting such surface antigens are well known and available to a skilled artisan.

In some embodiments, the NK cell population is enriched, such as by isolation or selection, from a sample by the provided methods are cells that are positive for (marker+ or marker^{pos}) or express high levels (marker^{high}) of one or more particular markers, such as surface markers, or that are negative for or express relatively low levels (marker− or marker^{neg}) of one or more markers. Hence, it is understood that the terms positive, pos or + with reference to a marker or protein expressed on or in a cell are used interchangeably herein. Likewise, it is understood that the terms negative, neg or − with reference to a marker or protein expressed on or in a cell are used interchangeably herein. Further, it is understood that reference to cells that are marker^{neg} herein may refer to cells that are negative for the marker as well as cells expressing relatively low levels of the marker, such as a low level that would not be readily detectable compared to control or background levels. In some cases, such markers are those that are absent or expressed at relatively low levels on certain populations of NK cells but are present or expressed at relatively higher levels on certain other populations of lymphocytes (such as T cells). In some cases, such markers are those that are present or expressed at relatively higher levels on certain populations of NK cells but are absent or expressed at relatively low levels on certain other populations of lymphocytes (such as T cells or subsets thereof).

In some embodiments, any known method for separation based on such markers may be used. In some embodiments, the separation is affinity- or immunoaffinity-based separation. For example, the isolation in some aspects includes separation of cells and cell populations based on the expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner. In some embodiments, incubation is static (without mixing). In some embodiments, incubation is dynamic (with mixing).

Such separation steps can be based on positive selection, in which the cells having bound the reagents are retained for further use, and/or negative selection, in which the cells having not bound to the antibody or binding partner are retained. In some examples, both fractions are retained for further use. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells. For example, in some aspects, a negative selection for CD3 enriches for a population of cells that are CD3^{neg}, but also can contain some residual or small percentage of other non-selected cells, which can, in some cases, include a small percentage of cells still being present in the enriched population that are CD3^{pos}. In some examples, a positive selection of one of the CD57^{pos} or CD16^{pos} population enriches for said population, either the CD57^{pos} or CD16^{pos} population, but also can contain some residual or small percentage of other non-selected cells, which can, in some cases, include the other of the CD57 or CD16 population still being present in the enriched population.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some aspects, the selection includes positive and/or negative selection steps based on expression of one or more of the surface antigens, such as in cells from a PBMC sample. In some embodiments, the isolation includes positive selection for cells expressing CD56, cells expressing CD16 or cells expressing CD57 and/or negative selection for cells expressing CD38 and/or negative selection for cells expressing non-NK cell markers, such as T cell markers, for example, negative selection for cells expressing CD3 (CD3^{neg}). For example, in some embodiments, the isolation includes positive selection for cells expressing CD56, cells expressing CD16 or cells expressing CD57 and/or negative selection for cells expressing non-NK cell markers, such as T cell markers, for example, negative selection for cells expressing CD3 (CD3^{neg}). In some embodiments, the isolation includes positive selection for cells expressing CD56, cells expressing CD16 or cells expressing CD57, and/or negative selection for cells expressing CD38 (CD38^{neg}), CD161 (CD161^{neg}), NKG2A (NKG2A^{neg}), and/or negative selection for cells expressing CD3 (CD3^{neg}). In some embodiments, the selection includes isolation of cells negative for CD3 (CD3^{neg}).

In some embodiments, the isolation includes negative selection for cells expressing CD3 (CD3^{neg}) and positive selection for cells expressing CD56 (CD56^{pos}). In some embodiments, the selection can further include negative selection for cells expressing CD38 (CD38^{neg}). In specific embodiments, the isolated or selected cells are CD3^{neg}CD56^{pos}CD38^{neg}.

In some embodiments, the selection includes negative selection for cells expressing CD3 (CD3^{neg}), positive selection for cells expressing CD56 (CD56^{pos}), followed by negative selection for cells expressing NKG2A (NKG2A^{neg}) and CD161 (CD161^{neg}). In specific embodiments, the isolated or selected cells are CD3^{neg}CD56^{pos}NKG2A^{neg} CD161^{neg}.

In some embodiments, the selection includes negative selection for cells expressing CD3 (CD3^{neg}) and positive selection for cells expressing CD57 (CD57^{pos}). In specific embodiments, the isolated or selected cells are CD3^{neg}CD57^{pos}.

In some embodiments, the selection includes negative selection for cells expressing CD3 (CD3^{neg}) and positive for cells expressing CD16 (CD16^{pos}). In specific embodiments, the isolated or selected cells are CD3^{neg}CD16^{pos}.

In some embodiments, the selection includes negative selection for cells expressing CD3 (CD3^{neg}) and positive selection for cells expressing CD57 (CD57^{pos}). In specific embodiments, the isolated or selected cells are CD3^{neg}CD57^{pos}. For example, the NK cells may be enriched by depletion of CD3^{pos} cells (negative selection for CD3^{pos} cells) followed by CD57^{pos} cell selection, thereby isolating and enriching CD57^{pos} NK cells. The separation can be carried out by immunoaffinity-based methods, such as using MACS™ Microbeads. For example, CD3 microbeads can be used to deplete CD3^{pos} cells in a negative selection for CD3^{neg} cells. Subsequently, CD57 MicroBeads can be used for CD57 enrichment of CD3 cell-depleted PBMCs. The CD3^{neg}/CD57^{pos} enriched NK cells can then be used in expansion in the provided methods.

In some embodiments, the selection may further include positive selection for cells expressing NKG2C (NKG2C^{pos}) and/or negative selection for cells NKG2A (NKG2A^{neg}). In some embodiments, the selection includes negative selection for cells expressing CD3 (CD3^{neg}), positive selection for cells expressing CD57 (CD57^{pos}), and positive selection for cells expressing NKG2C (NKG2C^{pos}). In specific embodiments, the isolated or selected cells are CD3^{neg}CD57^{pos}NKG2C^{pos}. In some embodiments, the selection includes negative selection for cells expressing CD3 (CD3^{neg}), positive selection for cells expressing CD57 (CD57^{pos}), and negative selection for cells expressing NKG2A (NKG2A^{neg}). In specific embodiments, the isolated or selected cells are CD3^{neg}CD57^{pos}NKG2A^{neg}. In some embodiments, the selection includes negative selection for cells expressing CD3 (CD3^{neg}), positive selection for cells expressing CD57 (CD57^{pos}), positive selection for cells expressing NKG2C (NKG2C^{pos}), and negative selection for cells expressing NKG2A (NKG2A^{neg}). In specific embodiments, the isolated or selected cells are CD3^{neg}CD57^{pos}NKG2C^{pos}NKG2A^{neg}.

In some of any of the provided embodiments, the selection can further include negative selection for cells expressing CD38 (CD38^{neg}). In specific embodiments, the isolated or selected cells are CD3^{neg}CD57^{pos}CD38^{neg}. In specific embodiments, the isolated or selected cells are CD3^{neg}CD57^{pos}CD38^{neg}NKG2C^{pos}. In specific embodiments, the isolated or selected cells are CD3^{neg}CD57^{pos}CD38^{neg}NKG2A^{neg}. In specific embodiments, the isolated or selected cells are CD3^{neg}CD57^{pos}CD38^{neg}NKG2C^{pos}NKG2A^{neg}.

In some embodiments, the selection includes negative selection for cells expressing CD3 (CD3^{neg}) and positive selection for cells expressing CD56 (CD56^{pos}). In specific embodiments, the isolated or selected cells are CD3^{neg}CD56^{pos}. In some embodiments, the selection includes negative selection for cells expressing CD3 (CD3^{neg}), positive selection for cells expressing CD56 (CD56^{pos}), and positive selection for cells expressing NKG2C (NKG2C^{pos}). In specific embodiments, the isolated or selected cells are CD3^{neg}CD56^{pos}NKG2C^{pos}. In some embodiments, the selection includes negative selection for cells expressing CD3 (CD3^{neg}), positive selection for cells expressing CD56 (CD56^{pos}), and negative selection for cells expressing NKG2A (NKG2A^{neg}). In specific embodiments, the isolated or selected cells are CD3^{neg}CD56^{pos}NKG2A^{neg}. In some embodiments, the selection includes negative selection for cells expressing CD3 (CD3^{neg}), positive selection for cells expressing CD56 (CD56^{pos}), positive selection for cells expressing NKG2C (NKG2C^{pos}), and negative selection for cells expressing NKG2A (NKG2A^{neg}). In specific embodiments, the isolated or selected cells are CD3^{neg}CD56^{pos}NKG2C^{pos}NKG2A^{neg}.

In some of any of the provided embodiments, the selection can further include negative selection for cells expressing CD38 (CD38^{neg}). In specific embodiments, the isolated or selected cells are CD3^{neg}CD56^{pos}CD38^{neg}. In specific embodiments, the isolated or selected cells are CD3^{neg}CD56^{pos}CD38^{neg}NKG2C^{pos}. In specific embodiments, the isolated or selected cells are CD3^{neg}CD56^{pos}CD38^{neg}NKG2A^{neg}. In specific embodiments, the isolated or selected cells are CD3^{neg}CD56^{pos}CD38^{neg}NKG2C^{pos}NKG2A^{neg}.

In some of any of the provided embodiments, the g-NK cells are cells having a g-NK surrogate surface marker profile. In some embodiments, the g-NK cell surrogate surface marker profile is CD16^{pos}/CD57^{pos}/CD7^{dim/neg}/CD161^{neg}. In some embodiments, the g-NK cell surrogate surface marker profile is NKG2A^{neg}/CD161^{neg}. In some of any such embodiments, the g-NK cell surrogate surface marker profile is CD38^{neg}. In some of any such embodiments, CD45^{pos}/CD3^{neg}/CD56^{pos} is used as a surrogate surface marker profile for NK cells. In some of any such embodiments, the g-NK cell surrogate surface marker profile further includes an NK cell surrogate surface marker profile. In some of any such embodiments, the g-NK cell surrogate surface marker profile further includes CD45^{pos}/CD3^{neg}/CD56^{pos}. In particular embodiments the g-NK cell surrogate surface marker profile includes CD45^{pos}/CD3^{neg}/CD56^{pos}/CD16^{pos}/CD57^{pos}/CD7^{dim/neg}/CD161^{neg}. In other particular embodiments, the g-NK cell surrogate surface marker profile includes CD45^{pos}/CD3^{neg}/CD56^{pos}/NKG2A^{neg}/CD161^{neg}. In other particular embodiments, the g-NK cell surrogate surface marker profile includes CD45^{pos}/CD3^{neg}/CD56^{pos}/CD38^{neg}.

In some embodiments, the methods of isolating, selecting and/or enriching for cells, such as by positive or negative selection based on the expression of a cell surface marker or markers, can include immunoaffinity-based selections. In some embodiments, the immunoaffinity-based selections include contacting a sample containing cells, such as PBMCs, with an antibody or binding partner that specifically binds to the cell surface marker or markers. In some embodiments, the antibody or binding partner is bound to a solid support or matrix, such as a sphere or bead, for example microbeads, nanobeads, including agarose, magnetic bead or paramagnetic beads, to allow for separation of cells for positive and/or negative selection. In some embodiments, the spheres or beads can be packed into a column to effect immunoaffinity chromatography, in which a sample containing cells, such as PBMCs, is contacted with the matrix of the column and subsequently eluted or released therefrom.

The incubation generally is carried out under conditions whereby the antibodies or binding partners, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In some embodiments, the magnetically responsive particles are left attached to the cells that are to be subsequently incubated and/or cultured; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the affinity-based selection is via magnetic-activated cell sorting (MACS) (Miltenyi Biotech, Auburn, CA). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In some of any of such embodiments, the method comprises administering IL-12, IL-15, IL-18, IL-2 and/or CCL5 to the subject prior to enriching, such as selecting and/or isolating, the NK cells or subset thereof.

B. Methods of Expanding NK Cells Enriched in g-NK Cells

In embodiments of the provided methods, the enriched NK cells are incubated or cultured in the presence of feeder cells, such as under conditions to support the proliferation and expansion of NK cell subsets, and in particular the g-NK cell subset.

In particular aspects, the feeder cells include cells that stimulate or promote expansion of NKG2C^{pos} and/or inhibit expansion of NKG2A^{pos} cells. In some embodiments, the feeder cells are cells that express or are transfected with HLA-E or a hybrid HLA-E containing the HLA-A2 signal sequence. For example, exemplary of such a hybrid is an AEH hybrid gene containing an MHC class I, such as HLA-A2, promoter and signal sequence and the HLA-E mature protein sequence, which, in some cases, can result in a mature protein identical to that encoded by the HLA-E gene but that can be stably expressed on the cell surface (see e.g. Lee et al. (1998) Journal of Immunology, 160:4951-4960). In some embodiments, the cell is an LCL 721.221, K562 cell or RMA-S cell that is transfected to express an MHC-E molecule stabilized in the presence of an MHC class I, such as HLA-A2, leader sequence. Cells lines that are engineered to express cell surface HLA-E stabilized in the presence of an MHC class I, such as HLA-A2, leader sequence peptide are known in the art (Lee et al. (1998) Journal of Immunology, 160:4951-4960; Zhongguo et al. (2005) 13:464-467; Garcia et al. (2002) Eur J. Immunol., 32:936-944). In some embodiments, 221.AEH cells, such as irradiated 221.AEH cells, can be used as feeder cells, or any other HLA-E-expressing cell line or irradiated HLA-E-expressing cell line that is otherwise HLA negative, such as K562. In some embodiments, the cell line can be transfected to express HLA-E. In some embodiments, K562 cells expressing membrane-bound IL-15 (K562-mb15) or membrane-bound IL-21 (K562-mb21) can be used as feeder cells. Exemplary of such a cell line for use in the methods provided herein are 221-AEH cells.

In embodiments, the HLA-expressing feeder cells are cryopreserved and thawed before use. In some embodiments, if the cells have been transfected to express HLA-E such as 221.AEH cells, the cells can be grown in the presence of appropriate nutrients, e.g. including serum or other appropriate serum replacement, and a selection agent prior to their use in the method. For example, in the case of 221.AEH cells, the cells can be cultured in cell culture media supplemented with Hygromycin B (e.g. 0.1% to 10%, such as at or about 1%) to maintain selective pressure on the cells to maintain the high level of plasmid HLA-E. The cells can be maintained at a density of 1×10⁵ cells/mL to 1×10⁶ cells/mL until use.

In particular embodiments, the HLA-E-expressing feeder cells, e.g. 221.AEH cells, added to the culture are non-dividing, such as by X-ray irradiation or gamma irradiation. The HLA-E-expressing feeder cells, e.g. 221.AEH, can be irradiated on the day of or just prior to their use in the provided methods. In some embodiments, the HLA-E-expressing feeder cells are irradiated with gamma rays in the range of about 1000 to 10000 rad, such as 1000-5000, rads to prevent cell division. In some embodiments, the HLA-E-expressing feeder cells are irradiated with gamma rays in the range of about 10 Gy to 100 Gy, such as 10-50 Gy to prevent cell division. In some embodiments, the cells are irradiated at 100 Gy. In other embodiments, irradiation is carried out by x-ray irradiation. In some embodiments, the HLA-E-expressing feeder cells are irradiated with x rays in the range of about 10 Gy to 100 Gy, such as 10-50 Gy to prevent cell division. In some embodiments, the A Rad-Sure™ blood irradiation indicator can be used to provide positive visual verification of irradiation. In aspects of the provided methods, the feeder cells are never removed; as a result of the irradiation the NK cells will be directly cytotoxic to the feeder cells and the feeder cells will die during the culture.

In some embodiments, the enriched, selected and/or isolated NK cells are incubated or cultured in the presence of HLA-E-expressing feeder cells (e.g. 221.AEH cells), such as an irradiated population thereof, at a ratio of feeder cells to enriched NK cells that is greater than or about 1:10 HLA-E feeder cells (e.g. 221.AEH cells), such as an irradiated population thereof, to enriched NK cells, such as from at or about 1:10 and at or about 10:1 of such feeder cells to enriched NK cells.

In some embodiments, the ratio of HLA-E-expressing feeder cells (e.g. 221.AEH cells), such as an irradiated population thereof, is at a ratio of such feeder cells to enriched NK cells that is between at or about 1:10 and at or about 10:1, between at or about 1:10 and at or about 5:1, between at or about 1:10 and at or about 2.5:1, between at or about 1:10 and at or about 1:1, between at or about 1:10 and at or about 1:2.5, between at or about 1:10 and at or about 1:5, between at or about 1:5 and at or about 10:1, between at or about 1:5 and at or about 5:1, between at or about 1:5 and at or about 2.5:1, between at or about 1:5 and at or about 1:1, between at or about 1:5 and at or about 1:2.5, between at or about 1:2.5 and at or about 10:1, between at or about 1:2.5 and at or about 5:1, between at or about 1:2.5 and at or about 2.5:1, between at or about 1:2.5 and at or about 1:1, between at or about 1:1 and at or about 10:1, between at or about 1:1 and at or about 5:1, between at or about 1:1 and at or about 3:1, between at or about 1:1 and at or about 2.5:1, between at or about 2.5:1 and at or about 10:1, between at or about 2.5:1 and at or about 5:1 or between at or about 5:1 and at or about 10:1, each inclusive.

In some embodiments, the ratio of HLA-expressing feeder cells (e.g. 221.AEH cells), such as an irradiated population thereof, is at a ratio of such feeder cells to enriched NK cells that is at or about 1.25:1, 1.5:1, 1.75:1, 2.0:1, 2.25:1, 2:5:1, 2.75:1, 3.0:1, 3.25:1, 3.5.:1, 3.75:1, 4.0:1, 4.25:1, 4.5:1, 4.75:1 or 5:1, or any value between any of the foregoing. In some embodiments, the ratio of HLA-expressing feeder cells (e.g. 221.AEH cells), such as an irradiated population thereof, is at a ratio of such feeder cells to enriched cells that is less than or less than about 5:1. In some embodiments, the ratio of HLA-expressing feeder cells (e.g. 221.AEH cells), such as an irradiated population thereof, is at a ratio between at or about 1:1 and 2.5:1, inclusive. In some embodiments, the ratio of HLA-expressing feeder cells (e.g. 221.AEH cells), such as an irradiated population thereof, is at a ratio of at or about 2.5:1. In some embodiments, the ratio of HLA-expressing feeder cells (e.g. 221.AEH cells), such as an irradiated population thereof, is at a ratio of at or about 2:1.

In some cases if the starting NK cell population has been cryopreserved prior to expansion, i.e. subject to freeze/thaw, a lower 221.AEH to NK-cell ratio can be employed than for methods using fresh NK cells. It is found here that a ratio of 1:1 221.AEH to freeze/thaw NK-cell resulted in comparable expansion in a culture containing a ratio of 2.5:1 221.AEH to fresh NK cells. In some aspects, the lower ratio ensures a higher number of NK cells in the culture to permit more cell-to-cell contact, which may play a role in promoting initial growth and expansion. In some embodiments, if initial enriched population of NK cells from a sample has been subject to freeze/thaw, a ratio of at or about 2:1 to 1:2 221.AEH to freeze/thaw NK-cells is used. In particular embodiments, the ratio is 1:1. It is understood that higher ratio, such as 2.5:1 221.AEH to freeze/thaw NK-cells can be used, but this may require a longer culture, e.g. at or about 21 days, to reach a desired threshold density or number.

In some embodiments, the NK cells are expanded by further adding to the culture non-dividing peripheral blood mononuclear cells (PBMC). In some aspects, the non-dividing feeder cells can comprise X-ray-irradiated PBMC feeder cells. In some aspects, the non-dividing feeder cells can comprise gamma-irradiated PBMC feeder cells. In some embodiments, the PBMC are irradiated with gamma rays in the range of about 1000 to 10000 rad, such as 1000-5000, rads to prevent cell division. In some embodiments, the PBMC are irradiated with gamma rays in the range of about 10 Gy to 100 Gy, such as 10-50 Gy to prevent cell division. In some aspects, during at least a portion of the incubation, the irradiated feeder cells are present in the culture medium at the same time as the non-dividing (e.g. irradiated) HLA-E-expressing feeder cells. In some aspects, the non-dividing (e.g. irradiated) PBMC feeder cell, HLA-E-expressing feeder cells and enriched NK cells are added to the culture on the same day, such as on the day of the initiation of the incubation, e.g. at or about or near the same time.

In some embodiments, the incubation or culture is further carried out in the presence of irradiated PBMCs as feeder cells. In some embodiments, the irradiated PBMC feeder cells are autologous to, or from the same subject as, the enriched NK cells were isolated or selected. In particular embodiments, the PBMCs are obtained from the same biological sample, e.g. whole blood or leukapheresis or apheresis product, as used to enrich the NK cells. Once obtained, a portion of the PBMCs are reserved for irradiation prior to enrichment of NK cells as described above.

In some embodiments, irradiated PBMCs are present as feeder cells at a ratio of such feeder cells to enriched NK cells that is from at or about 1:10 to at or about 10:1, from at or about 1:10 to at or about 5:1, from at or about 1:10 to at or about 2.5:1, from at or about 1:10 to at or about 1:1, from at or about 1:10 to at or about 1:2.5, from at or about 1:10 to at or about 1:5, from at or about 1:5 to at or about 10:1, from at or about 1:5 to at or about 5:1, from at or about 1:5 to at or about 2.5:1, from at or about 1:5 to at or about 1:1, from at or about 1:5 to at or about 1:2.5, from at or about 1:2.5 to at or about 10:1, from at or about 1:2.5 to at or about 5:1, from at or about 1:2.5 to at or about 2.5:1, from at or about 1:2.5 to at or about 1:1, from at or about 1:1 to at or about 10:1, from at or about 1:1 to at or about 5:1, from at or about 1:1 to at or about 2.5:1, from at or about 2.5:1 to at or about 10:1, from at or about 2.5:1 to at or about 5:1 or from at or about 5:1 to at or about 10:1.

In some embodiments, the irradiated PBMCs are present as feeder cells at a ratio of such feeder cells to enriched NK cells that is between at or about 1:1 and at or about 5:1, such as at or about 1.25:1, 1.5:1, 1.75:1, 2.0:1, 2.25:1, 2:5:1, 2.75:1, 3.0:1, 3.25:1, 3.5.:1, 3.75:1, 4.0:1, 4.25:1, 4.5:1, 4.75:1 or 5:1, or any value between any of the foregoing. In some embodiments, the irradiated PBMCs are present at a ratio of such feeder cells to enriched cells that is or is about 5:1.

In particular embodiments, during at least a portion of the incubation or culture one or more cells or cell types, such as T cells, of the irradiated PBMCs are activated and/or the incubation or culture is carried out in the presence of at least one stimulatory agent that is capable of stimulating the activation of one or more T cells of the PBMC feeder cells. In some embodiments, at least one stimulatory agent specifically binds to a member of a TCR complex. In some embodiments, the at least one stimulatory agent specifically binds to a CD3, optionally a CD3epsilon. In some aspects, the at least one stimulatory agent is an anti-CD3 antibody or antigen binding fragment. An exemplary anti-CD3 antibody includes mouse anti-human CD3 (OKT3).

In some embodiments, the anti-CD3 antibody or antigen-binding fragment is present during at least a portion of the incubation that includes irradiated PBMC feeder cells. In some embodiments, the anti-CD3 antibody or antigen-binding fragment is added to the culture or incubation at or about the same time as the irradiated PBMCs. For example, the anti-CD3 antibody or antigen-binding fragment is added at or about at the initiation of the incubation or culture. In particular aspects, the anti-CD3 antibody or antigen-binding fragment may be removed, or its concentration reduced, during the course of the culture or incubation, such as by exchanging or washing out the culture medium. In particular embodiments, after exchanging or washing, the methods do not include adding back or replenishing the culture media with the anti-CD3 antibody or antigen-binding fragment.

In some embodiments, the anti-CD3 antibody or antigen-binding fragment is added, or is present during at least a portion of the culture or incubation, at a concentration that is between at or about 10 ng/mL and at or about 5 μg/mL, such as between at or about 10 ng/mL and at or about 2 μg/mL, between at or about 10 ng/mL and at or about 1 μg/mL, between at or about 10 ng/mL and at or about 500 ng/mL, between at or about 10 ng/mL and at or about 100 ng/mL, between at or about 10 ng/mL and at or about 50 ng/mL, between at or about 50 ng/mL and at or about 5 μg/mL, such as between at or about 50 ng/mL and at or about 2 μg/mL, between at or about 50 ng/mL and at or about 1 μg/mL, between at or about 50 ng/mL and at or about 500 ng/mL, between at or about 50 ng/mL and at or about 100 ng/mL, between at or about 100 ng/mL and at or about 5 μg/mL, between at or about 100 ng/mL and at or about 2 μg/mL, between at or about 100 ng/mL and at or about 1 μg/mL, between at or about 100 ng/mL and at or about 500 ng/mL, between at or about 500 ng/mL and at or about 5 μg/mL, between at or about 500 ng/mL and at or about 2 μg/mL, between at or about 500 ng/mL and at or about 1 μg/mL, between at or about 1 μg/mL and at or about 5 μg/mL, between at or about 1 μg/mL and at or about 2 μg/mL, or between at or about 2 μg/mL and at or about 5 μg/mL, each inclusive. In some embodiments, the concentration of the anti-CD3 antibody or antigen-binding fragment is at or about 10 ng/mL, 20 ng/mL, 30 ng/mL, 40 ng/mL, 50 ng/mL, 60 ng/mL, 70 ng/mL, 80 ng/mL, 90 ng/mL or 100 ng/mL, or any value between any of the foregoing. In some embodiments, the concentration of the anti-CD3 antibody or antigen-binding fragment is or is about 50 ng/mL.

In some embodiments, the term “antibody” refers to immunoglobulin molecules and antigen-binding portions or fragments of immunoglobulin (Ig) molecules, i.e., molecules that contain an antigen binding site that specifically binds (immunoreacts with) an antigen. The term antibody encompasses not only intact polyclonal or monoclonal antibodies, but also fragments thereof, such as dAb, Fab, Fab′, F(ab′)₂, Fv), single chain (scFv) or single domain antibody (sdAb). Typically, an “antigen-binding fragment” contains at least one CDR of an immunoglobulin heavy and/or light chain that binds to at least one epitope of the antigen of interest. In this regard, an antigen-binding fragment may comprise 1, 2, 3, 4, 5, or all 6 CDRs of a variable heavy chain (VH) and variable light chain (VL) sequence from antibodies that bind the antigen, such as generally six CDRs for an antibody containing a VH and a VL (“CDR1,” “CDR2” and “CDR3” for each of a heavy and light chain), or three CDRs for an antibody containing a single variable domain.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; variable heavy chain (V_{H}) regions, single-chain antibody molecules such as scFvs and single-domain V_{H} single antibodies; and multispecific antibodies formed from antibody fragments. In particular embodiments, the antibodies are single-chain antibody fragments comprising a variable heavy chain region and/or a variable light chain region, such as scFvs.

In some embodiments, the incubation or culture is initiated in the presence of such enriched NK cells, such as selected and/or isolated NK cells, at a concentration that is at or about, or at least at or about, 0.05×10⁶ enriched NK cells/mL, at or about 0.1×10⁶ enriched NK cells/mL, at or about 0.2×10⁶ enriched NK cells/mL, at or about 0.5×10⁶ enriched NK cells/mL or at or about 1.0×10⁶ enriched NK cells/mL. In embodiments of the provided methods, the incubation or culture is initiated in the presence of such enriched NK cells, such as selected and/or isolated NK cells, at a concentration that is between at or about 0.05×10⁶ enriched NK cells/mL and at or about 1.0×10⁶ enriched NK cells/mL, such as between at or about 0.05×10⁶ enriched NK cells/mL and at or about 0.75×10⁶, between at or about 0.05×10⁶ enriched NK cells/mL and at or about 0.5×10⁶, between at or about 0.05×10⁶ enriched NK cells/mL and at or about 0.20×10⁶ enriched NK cells/mL, between at or about 0.05×10⁶ enriched NK cells/mL and at or about 0.1×10⁶ enriched NK cells/mL, between at or about 0.1×10⁶ enriched NK cells/mL and at or about 1.0×10⁶ enriched NK cells/mL, between at or about 0.1×10⁶ enriched NK cells/mL and at or about 0.75×10⁶, between at or about 0.1×10⁶ enriched NK cells/mL and at or about 0.5×10⁶, between at or about 0.1×10⁶ enriched NK cells/mL and at or about 0.20×10⁶ enriched NK cells/mL, between at or about 0.20×10⁶ enriched NK cells/mL and at or about 1.0×10⁶ enriched NK cells/mL, between at or about 0.20×10⁶ enriched NK cells/mL and at or about 0.75×10⁶, between at or about 0.20×10⁶ enriched NK cells/mL and at or about 0.5×10⁶, between at or about 0.5×10⁶ enriched NK cells/mL and at or about 1.0×10⁶ enriched NK cells/mL, between at or about 0.5×10⁶ enriched NK cells/mL and at or about 0.75×10⁶, between at or about 0.75×10⁶ enriched NK cells/mL and at or about 1.0×10⁶ enriched NK cells/mL, each inclusive. In some embodiments, the incubation or culture is initiated in the presence of such enriched NK cells, such as selected and/or isolated NK cells, at a concentration that is at or about 0.2×10⁶ enriched NK cells/mL.

In some of any such embodiments, the amount of enriched NK cells, such as selected or isolated from PBMCs as described above in Section V. A, added or present at the initiation of the incubation or culture is at least or at least about 1×10⁵ cells, at least or at least about 2×10⁵ cells, at least or at least about 3×10⁵ cells, at least or at least about 4×10⁵ cells, at least or at least about 5×10⁵ cells, at least or at least about 6×10⁵ cells, at least or at least about 7×10⁵ cells, at least or at least about 8×10⁵ cells, at least or at least about 9×10⁵ cells, at least or at least about 1×10⁶ cells or more. In particular embodiments, the amount of enriched NK cells, such as selected or isolated from PBMCs as described above, is at least or about at least or is or is about 1×10⁶ cells.

In some embodiments, at the initiation of the incubation or culture the population of enriched NK cells, such as selected or isolated as described above in Section V. A, comprises at least at or about 2.0×10⁶ enriched NK cells, at least at or about 3.0×10⁶ enriched NK cells, at least at or about 4.0×10⁶ enriched NK cells, at least at or about 5.0×10⁶ enriched NK cells, at least at or about 6.0×10⁶ enriched NK cells, at least at or about 7.0×10⁶ enriched NK cells, at least at or about 8.0×10⁶ enriched NK cells, at least at or about 9.0×10⁶ enriched NK cells, at least at or about 1.0×10⁷ enriched NK cells, at least at or about 5.0×10⁷ enriched NK cells, at least at or about 1.0×10⁸ enriched NK cells, at least at or about 5.0×10⁸ enriched NK cells, or at least at or about 1.0×10⁹ enriched NK cells. In some embodiments, the population of enriched NK cells comprises at least at or about 2.0×10⁵ enriched NK cells. In some embodiments, the population of enriched NK cells comprises at least at or about 1.0×10⁶ enriched NK cells. In some embodiments, the population of enriched NK cells comprises at least at or about 1.0×10⁷ enriched NK cells.

In some embodiments, at the initiation of the incubation or culturing the population of enriched NK cells, such as selected or isolated as described in Section V. A, comprises between at or about 2.0×10⁵ enriched NK cells and at or about 1.0×10⁹ enriched NK cells, between at or about 2.0×10⁵ enriched NK cells and at or about 5.0×10⁸ enriched NK cells, between at or about 2.0×10⁵ enriched NK cells and at or about 1.0×10⁸ enriched NK cells, between at or about 2.0×10⁵ enriched NK cells and at or about 5.0×10⁷ enriched NK cells, between at or about 2.0×10⁵ enriched NK cells and at or about 1.0×10⁷ enriched NK cells, between at or about 2.0×10⁵ enriched NK cells and at or about 5.0×10⁶ enriched NK cells, between at or about 2.0×10⁵ enriched NK cells and at or about 1.0×10⁶ enriched NK cells, between at or about 1.0×10⁶ enriched NK cells and at or about 1.0×10⁹ enriched NK cells, between at or about 1.0×10⁶ enriched NK cells and at or about 5.0×10⁸ enriched NK cells, between at or about 1.0×10⁶ enriched NK cells and at or about 1.0×10⁸ enriched NK cells, between at or about 1.0×10⁶ enriched NK cells and at or about 5.0×10⁷ enriched NK cells, between at or about 1.0×10⁶ enriched NK cells and at or about 1.0×10⁷ enriched NK cells, between at or about 1.0×10⁶ enriched NK cells and at or about 5.0×10⁶ enriched NK cells, between at or about 5.0×10⁶ enriched NK cells and at or about 1.0×10⁹ enriched NK cells, between at or about 5.0×10⁶ enriched NK cells and at or about 5.0×10⁸ enriched NK cells, between at or about 5.0×10⁶ enriched NK cells and at or about 1.0×10⁸ enriched NK cells, between at or about 5.0×10⁶ enriched NK cells and at or about 5.0×10⁷ enriched NK cells, between at or about 5.0×10⁶ enriched NK cells and at or about 1.0×10⁷ enriched NK cells, between at or about 1.0×10⁷ enriched NK cells and at or about 1.0×10⁹ enriched NK cells, between at or about 1.0×10⁷ enriched NK cells and at or about 5.0×10⁸ enriched NK cells, between at or about 1.0×10⁷ enriched NK cells and at or about 1.0×10⁸ enriched NK cells, between at or about 1.0×10⁷ enriched NK cells and at or about 5.0×10⁷ enriched NK cells, between at or about 5.0×10⁷ enriched NK cells and at or about 1.0×10⁹ enriched NK cells, between at or about 5.0×10⁷ enriched NK cells and at or about 5.0×10⁸ enriched NK cells, between at or about 5.0×10⁷ enriched NK cells and at or about 1.0×10⁸ enriched NK cells, between at or about 1.0×10⁸ enriched NK cells and at or about 1.0×10⁹ enriched NK cells, between at or about 1.0×10⁸ enriched NK cells and at or about 5.0×10⁸ enriched NK cells, or between at or about 5.0×10⁸ enriched NK cells and at or about 1.0×10⁹ enriched NK cells. In some embodiments, at the initiation of the culturing or incubation the population of enriched NK cells comprises between at or about 2.0×10⁵ enriched NK cells and at or about 5.0×10⁷ enriched NK cells. In some embodiments, at the initiation of the culturing or incubation the population of enriched NK cells comprises between at or about 1.0×10⁶ enriched NK cells and at or about 1.0×10⁸ enriched NK cells. In some embodiments, at the initiation of the culturing or incubation the population of enriched NK cells comprises between at or about 1.0×10⁷ enriched NK cells and at or about 5.0×10⁸ enriched NK cells. In some embodiments, at the initiation of the culturing or incubation the population of enriched NK cells comprises between at or about 1.0×10⁷ enriched NK cells and at or about 1.0×10⁹ enriched NK cells.

In some embodiments, the percentage of g-NK cells among the population of enriched NK cells present at the initiation of the culturing or incubation is between at or about 20% and at or about 90%, between at or about 20% and at or about 80%, between at or about 20% and at or about 70%, between at or about 20% and at or about 60%, between at or about 20% and at or about 50%, between at or about 20% and at or about 40%, between at or about 20% and at or about 30%, between at or about 30% and at or about 90%, between at or about 30% and at or about 80%, between at or about 30% and at or about 70%, between at or about 30% and at or about 60%, between at or about 30% and at or about 50%, between at or about 30% and at or about 40%, between at or about 40% and at or about 90%, between at or about 40% and at or about 80%, between at or about 40% and at or about 70%, between at or about 40% and at or about 60%, between at or about 40% and at or about 50%, between at or about 50% and at or about 90%, between at or about 50% and at or about 80%, between at or about 50% and at or about 70%, between at or about 50% and at or about 60%, between at or about 60% and at or about 90%, between at or about 60% and at or about 80%, between at or about 60% and at or about 70%, between at or about 70% and at or about 90%, between at or about 70% and at or about 80%, or between at or about 80% and at or about 90%. In some embodiments, the percentage of g-NK cells among the population of enriched NK cells at the initiation of the culturing or incubation is between at or about 20% and at or about 90%. In some embodiments, the percentage of g-NK cells among the population of enriched NK cells at the initiation of the culturing or incubation is between at or about 40% and at or about 90%. In some embodiments, the percentage of g-NK cells among the population of enriched NK cells at the initiation of the culturing or incubation is between at or about 60% and at or about 90%.

In some of these embodiments, the NK cells can be cultured with a growth factor. According to some embodiments, the at least one growth factor comprises a growth factor selected from the group consisting of SCF, GSK3i, FLT3, IL-2, IL-6, IL-7, IL-15, IL-12, IL-18 and IL-21. According to some embodiments, the at least one growth factor is IL-2 or IL-7 and IL-15. According to some embodiments, the at least one growth factor is IL-2, IL-21 or IL-7 and IL-15. In some embodiments, the growth factor is a recombinant cytokine, such as a recombinant IL-2, recombinant IL-7, recombinant IL-21 or recombinant IL-15.

In some embodiments, the NK cells are cultured in the presence of one or more recombinant cytokines. In some embodiments, the one or more recombinant cytokines comprise any of SCF, GSK3i, FLT3, IL-2, IL-6, IL-7, IL-15, IL-12, IL-18, IL-21, IL-27, or combinations thereof. In some embodiments, the one or more recombinant cytokines comprise any of IL-2, IL-7, IL-15, IL-12, IL-18, IL-21, IL-27, or combinations thereof. In some embodiments, at least one of the one or more recombinant cytokines is IL-21. In some embodiments, the one or more recombinant cytokines further comprises IL-2, IL-7, IL-15, IL-12, IL-18, or IL-27, or combinations thereof. In some embodiments, at least one of the one or more recombinant cytokines is IL-2. In some embodiments, the one or more recombinant cytokines is at least IL-2 and IL-21. In some embodiments, the one or more recombinant cytokines are IL-21 and IL-2. In some embodiments, the one or more recombinant cytokines are IL-21, IL-2, and IL-15. In some embodiments, the one or more recombinant cytokines are IL-21, IL-12, IL-15, and IL-18. In some embodiments, the one or more recombinant cytokines are IL-21, IL-2, 11-12, IL-15, and IL-18. In some embodiments, the one or more recombinant cytokines are IL-21, IL-15, IL-18, and IL-27. In some embodiments, the one or more recombinant cytokines are IL-21, IL-2, IL-15, IL-18, and IL-27. In some embodiments, the one or more recombinant cytokines are IL-2 and IL-15.

In particular embodiments, the provided methods include incubation or culture of the enriched NK cells and feeder cells in the presence of recombinant IL-2. In some embodiments, during at least a portion of the incubation, e.g. added at the initiation of the culturing and optionally one or more times during the culturing, the recombinant IL-2 is present at a concentration of between at or about 1 IU/mL and at or about 500 IU/mL, such as between at or about 1 IU/mL and at or about 250 IU/mL, between at or about 1 IU/mL and at or about 100 IU/mL, between at or about 1 IU/mL and at or about 50 IU/mL, between at or about 50 IU/mL and at or about 500 IU/mL, between at or about 50 IU/mL and at or about 250 IU/mL, between at or about 50 IU/mL and at or about 100 IU/mL, between at or about 100 IU/mL and at or about 500 IU/mL, between at or about 100 IU/mL and at or about 250 IU/mL or between at or about 250 IU/mL and at or about 500 IU/mL, each inclusive. In some embodiments, during at least a portion of the incubation, e.g. added at the initiation of the culturing and optionally one or more times during the culturing, the concentration of the IL-2 is at or about 50 IU/mL, 60 IU/mL, 70 IU/mL, 80 IU/mL, 90 IU/mL, 100 IU/mL, 125 IU/mL, 150 IU/mL, 200 IU/mL, or any value between any of the foregoing. In particular embodiments, the concentration of the recombinant IL-2 added at the initiation of the culturing and optionally one or more times during the culturing is or is about 100 IU/mL. In particular embodiments, the concentration of the recombinant IL-2 added at the initiation of the culturing and optionally one or more times during the culturing is or is about 500 IU/mL.

In particular embodiments, the provided methods include incubation or culture of the enriched NK cells and feeder cells in the presence of recombinant IL-21. In some embodiments, during at least a portion of the incubation, e.g. added at the initiation of the culturing and optionally one or more times during the culturing, the recombinant IL-21 is present at a concentration of between at or about 1 IU/mL and at or about 500 IU/mL, such as between at or about 1 IU/mL and at or about 250 IU/mL, between at or about 1 IU/mL and at or about 100 IU/mL, between at or about 1 IU/mL and at or about 50 IU/mL, between at or about 50 IU/mL and at or about 500 IU/mL, between at or about 50 IU/mL and at or about 250 IU/mL, between at or about 50 IU/mL and at or about 100 IU/mL, between at or about 100 IU/mL and at or about 500 IU/mL, between at or about 100 IU/mL and at or about 250 IU/mL or between at or about 250 IU/mL and at or about 500 IU/mL, each inclusive. In some embodiments, during at least a portion of the incubation, e.g. added at the initiation of the culturing and optionally one or more times during the culturing, the concentration of the IL-21 is at or about 50 IU/mL, 60 IU/mL, 70 IU/mL, 80 IU/mL, 90 IU/mL, 100 IU/mL, 125 IU/mL, 150 IU/mL, 200 IU/mL, or any value between any of the foregoing. In particular embodiments, the concentration of the recombinant IL-21 added at the initiation of the culturing and optionally one or more times during the culturing, is or is about 100 IU/mL.

In particular embodiments, the provided methods include incubation or culture of the enriched NK cells and feeder cells in the presence of recombinant IL-21. In particular embodiments, the concentration of recombinant IL-21 during at least a portion of the culturing, e.g. added at the initiation of the culturing and optionally one or more times during the culturing, is between about 10 ng/mL and about 100 ng/mL, between about 10 ng/mL and about 90 ng/mL, between about 10 ng/mL and about 80 ng/mL, between about 10 ng/mL and about 70 ng/mL, between about 10 ng/mL and about 60 ng/mL, between about 10 ng/mL and about 50 ng/mL, between about 10 ng/mL and about 40 ng/mL, between about 10 ng/mL and about 30 ng/mL, between about 10 ng/mL and about 20 ng/mL, between about 20 ng/mL and about 100 ng/mL, between about 20 ng/mL and about 90 ng/mL, between about 20 ng/mL and about 80 ng/mL, between about 20 ng/mL and about 70 ng/mL, between about 20 ng/mL and about 60 ng/mL, between about 20 ng/mL and about 50 ng/mL, between about 20 ng/mL and about 40 ng/mL, between about 20 ng/mL and about 30 ng/mL, between about 30 ng/mL and about 100 ng/mL, between about 30 ng/mL and about 90 ng/mL, between about 30 ng/mL and about 80 ng/mL, between about 30 ng/mL and about 70 ng/mL, between about 30 ng/mL and about 60 ng/mL, between about 30 ng/mL and about 50 ng/mL, between about 30 ng/mL and about 40 ng/mL, between about 40 ng/mL and about 100 ng/mL, between about 40 ng/mL and about 90 ng/mL, between about 40 ng/mL and about 80 ng/mL, between about 40 ng/mL and about 70 ng/mL, between about 40 ng/mL and about 60 ng/mL, between about 40 ng/mL and about 50 ng/mL, between about 50 ng/mL and about 100 ng/mL, between about 50 ng/mL and about 90 ng/mL, between about 50 ng/mL and about 80 ng/mL, between about 50 ng/mL and about 70 ng/mL, between about 50 ng/mL and about 60 ng/mL, between about 60 ng/mL and about 100 ng/mL, between about 60 ng/mL and about 90 ng/mL, between about 60 ng/mL and about 80 ng/mL, between about 60 ng/mL and about 70 ng/mL, between about 70 ng/mL and about 100 ng/mL, between about 70 ng/mL and about 90 ng/mL, between about 70 ng/mL and about 80 ng/mL, between about 80 ng/mL and about 100 ng/mL, between about 80 ng/mL and about 90 ng/mL, or between about 90 ng/mL and about 100 ng/mL, inclusive. In particular embodiments, the concentration of recombinant IL-21 during at least a portion of the culturing, e.g. added at the initiation of the culturing and optionally one or more times during the culturing, is between about 10 ng/mL and about 100 ng/mL, inclusive. In particular embodiments, the concentration of recombinant IL-21 during at least a portion of the culturing, e.g. added at the initiation of the culturing and optionally one or more times during the culturing, is at or about 25 ng/mL.

In particular embodiments, the concentration of recombinant IL-15 during at least a portion of the culturing, e.g. added at the initiation of the culturing and optionally one or more times during the culturing, is between about 1 ng/mL and about 50 ng/mL, between about 1 ng/mL and about 40 ng/mL, between about 1 ng/mL and about 30 ng/mL, between about 1 ng/mL and about 20 ng/mL, between about 1 ng/mL and about 10 ng/mL, between about 1 ng/mL and about 5 ng/mL, between about 5 ng/mL and about 50 ng/mL, between about 5 ng/mL and about 40 ng/mL, between about 5 ng/mL and about 30 ng/mL, between about 5 ng/mL and about 20 ng/mL, between about 5 ng/mL and about 10 ng/mL, between about 10 ng/mL and about 50 ng/mL, between about 10 ng/mL and about 40 ng/mL, between about 10 ng/mL and about 30 ng/mL, between about 10 ng/mL and about 20 ng/mL, between about 20 ng/mL and about 50 ng/mL, between about 20 ng/mL and about 40 ng/mL, between about 20 ng/mL and about 30 ng/mL, between about 30 ng/mL and about 50 ng/mL, between about 30 ng/mL and about 40 ng/mL, or between about 40 ng/mL and about 50 ng/mL. In particular embodiments, the concentration of recombinant IL-15 during at least a portion of the culturing, e.g. added at the initiation of the culturing and optionally one or more times during the culturing, is between about 1 ng/mL and about 50 ng/mL. In particular embodiments, the concentration of recombinant IL-15 during at least a portion of the culturing, e.g. added at the initiation of the culturing and optionally one or more times during the culturing, is at or about 10 ng/mL.

In particular embodiments, the methods include culture in the presence of IL-2, IL-15 and IL-21. In embodiments of the provided methods, the concentration of recombinant cytokines, e.g. added to the culture at the initiation of the culturing and optionally one or more times during the culturing, is at between at or about 50 IU/mL and at or about 500 IU/mL IL-2, such as at or about 100 IU/mL or 500 IU/mL IL-2; between at or about 1 ng/mL and 50 ng/mL IL-15, such as at or about 10 ng/mL; and between at or about 10 ng/mL and at or about 100 ng/mL IL-21, such as at or about 25 ng/mL. In particular embodiments, 500 IU/mL of IL-2, 10 ng/mL of IL-15, and 25 ng/mL of IL-21 are added during at least a portion of the culturing, such as added at the initiation of the culturing and optionally one or more times during the culturing. In particular embodiments, 100 IU/mL of IL-2, 10 ng/mL of IL-15, and 25 ng/mL of IL-21 are added during at least a portion of the culturing, such as added at the initiation of the culturing and optionally one or more times during the culturing.

In some embodiments, the provided methods include incubation or culture of the enriched NK cells and feeder cells in the presence of recombinant IL-21 and the recombinant IL-21 is added as a complex with an anti-IL-21 antibody. In some embodiments, prior to the culturing, anti-IL-21 antibody is contacted with the recombinant IL-21, thereby forming an IL-21/anti-IL-21 complex, and the IL-21/anti-IL-21 complex is added to the culture medium. In some embodiments, contacting the recombinant IL-21 and the anti-IL-21 antibody to form an IL-21/anti-IL-21 complex is carried out under conditions that include temperature and time suitable for the formation of the complex. In some embodiments, the culturing is carried out at 37° C.±2 for 30 minutes.

In some embodiments, anti-IL-21 antibody is added at a concentration between at or about 100 ng/mL and at or about 500 ng/mL, between at or about 100 ng/mL and at or about 400 ng/mL, between at or about 100 ng/mL and at or about 300 ng/mL, between at or about 100 ng/mL and at or about 200 ng/mL, between at or about 200 ng/mL and at or about 500 ng/mL, between at or about 200 ng/mL and at or about 400 ng/mL, between at or about 200 ng/mL and at or about 300 ng/mL, between at or about 300 ng/mL and at or about 500 ng/mL, between at or about 300 ng/mL and at or about 400 ng/mL, or between at or about 400 ng/mL and at or about 500 ng/mL. In some embodiments, anti-IL-21 antibody is added at a concentration between at or about 100 ng/mL and at or about 500 ng/mL. In some embodiments, anti-IL-21 antibody is added at a concentration of 250 ng/mL.

In particular embodiments, the concentration of recombinant IL-21 used to form a complex with the anti-IL-21 antibody is between about 10 ng/mL and about 100 ng/mL, between about 10 ng/mL and about 90 ng/mL, between about 10 ng/mL and about 80 ng/mL, between about 10 ng/mL and about 70 ng/mL, between about 10 ng/mL and about 60 ng/mL, between about 10 ng/mL and about 50 ng/mL, between about 10 ng/mL and about 40 ng/mL, between about 10 ng/mL and about 30 ng/mL, between about 10 ng/mL and about 20 ng/mL, between about 20 ng/mL and about 100 ng/mL, between about 20 ng/mL and about 90 ng/mL, between about 20 ng/mL and about 80 ng/mL, between about 20 ng/mL and about 70 ng/mL, between about 20 ng/mL and about 60 ng/mL, between about 20 ng/mL and about 50 ng/mL, between about 20 ng/mL and about 40 ng/mL, between about 20 ng/mL and about 30 ng/mL, between about 30 ng/mL and about 100 ng/mL, between about 30 ng/mL and about 90 ng/mL, between about 30 ng/mL and about 80 ng/mL, between about 30 ng/mL and about 70 ng/mL, between about 30 ng/mL and about 60 ng/mL, between about 30 ng/mL and about 50 ng/mL, between about 30 ng/mL and about 40 ng/mL, between about 40 ng/mL and about 100 ng/mL, between about 40 ng/mL and about 90 ng/mL, between about 40 ng/mL and about 80 ng/mL, between about 40 ng/mL and about 70 ng/mL, between about 40 ng/mL and about 60 ng/mL, between about 40 ng/mL and about 50 ng/mL, between about 50 ng/mL and about 100 ng/mL, between about 50 ng/mL and about 90 ng/mL, between about 50 ng/mL and about 80 ng/mL, between about 50 ng/mL and about 70 ng/mL, between about 50 ng/mL and about 60 ng/mL, between about 60 ng/mL and about 100 ng/mL, between about 60 ng/mL and about 90 ng/mL, between about 60 ng/mL and about 80 ng/mL, between about 60 ng/mL and about 70 ng/mL, between about 70 ng/mL and about 100 ng/mL, between about 70 ng/mL and about 90 ng/mL, between about 70 ng/mL and about 80 ng/mL, between about 80 ng/mL and about 100 ng/mL, between about 80 ng/mL and about 90 ng/mL, or between about 90 ng/mL and about 100 ng/mL, inclusive. In particular embodiments, the concentration of recombinant IL-21 used to form a complex with the anti-IL-21 antibody is between about 10 ng/mL and about 100 ng/mL, inclusive. In particular embodiments, the concentration of recombinant IL-21 used to form a complex with the anti-IL-21 antibody is at or about 25 ng/mL.

In particular embodiments, the concentration of recombinant IL-12 during at least a portion of the culturing, e.g. added at the initiation of the culturing and optionally one or more times during the culturing, is between about 1 ng/mL and about 50 ng/mL, between about 1 ng/mL and about 40 ng/mL, between about 1 ng/mL and about 30 ng/mL, between about 1 ng/mL and about 20 ng/mL, between about 1 ng/mL and about 10 ng/mL, between about 1 ng/mL and about 5 ng/mL, between about 5 ng/mL and about 50 ng/mL, between about 5 ng/mL and about 40 ng/mL, between about 5 ng/mL and about 30 ng/mL, between about 5 ng/mL and about 20 ng/mL, between about 5 ng/mL and about 10 ng/mL, between about 10 ng/mL and about 50 ng/mL, between about 10 ng/mL and about 40 ng/mL, between about 10 ng/mL and about 30 ng/mL, between about 10 ng/mL and about 20 ng/mL, between about 20 ng/mL and about 50 ng/mL, between about 20 ng/mL and about 40 ng/mL, between about 20 ng/mL and about 30 ng/mL, between about 30 ng/mL and about 50 ng/mL, between about 30 ng/mL and about 40 ng/mL, or between about 40 ng/mL and about 50 ng/mL. In particular embodiments, the concentration of recombinant IL-12 during at least a portion of the culturing, e.g. added at the initiation of the culturing and optionally one or more times during the culturing, is between about 1 ng/mL and about 50 ng/mL. In particular embodiments, the concentration of recombinant IL-12 during at least a portion of the culturing, e.g. added at the initiation of the culturing and optionally one or more times during the culturing, is at or about 10 ng/mL.

In particular embodiments, the concentration of recombinant IL-18 during at least a portion of the culturing, e.g. added at the initiation of the culturing and optionally one or more times during the culturing, is between about 1 ng/mL and about 50 ng/mL, between about 1 ng/mL and about 40 ng/mL, between about 1 ng/mL and about 30 ng/mL, between about 1 ng/mL and about 20 ng/mL, between about 1 ng/mL and about 10 ng/mL, between about 1 ng/mL and about 5 ng/mL, between about 5 ng/mL and about 50 ng/mL, between about 5 ng/mL and about 40 ng/mL, between about 5 ng/mL and about 30 ng/mL, between about 5 ng/mL and about 20 ng/mL, between about 5 ng/mL and about 10 ng/mL, between about 10 ng/mL and about 50 ng/mL, between about 10 ng/mL and about 40 ng/mL, between about 10 ng/mL and about 30 ng/mL, between about 10 ng/mL and about 20 ng/mL, between about 20 ng/mL and about 50 ng/mL, between about 20 ng/mL and about 40 ng/mL, between about 20 ng/mL and about 30 ng/mL, between about 30 ng/mL and about 50 ng/mL, between about 30 ng/mL and about 40 ng/mL, or between about 40 ng/mL and about 50 ng/mL. In particular embodiments, the concentration of recombinant IL-18 during at least a portion of the culturing, e.g. added at the initiation of the culturing and optionally one or more times during the culturing, is between about 1 ng/mL and about 50 ng/mL. In particular embodiments, the concentration of recombinant IL-18 during at least a portion of the culturing, e.g. added at the initiation of the culturing and optionally one or more times during the culturing, is at or about 10 ng/mL.

In particular embodiments, the concentration of recombinant IL-27 during at least a portion of the culturing, e.g. added at the initiation of the culturing and optionally one or more times during the culturing, is between about 1 ng/mL and about 50 ng/mL, between about 1 ng/mL and about 40 ng/mL, between about 1 ng/mL and about 30 ng/mL, between about 1 ng/mL and about 20 ng/mL, between about 1 ng/mL and about 10 ng/mL, between about 1 ng/mL and about 5 ng/mL, between about 5 ng/mL and about 50 ng/mL, between about 5 ng/mL and about 40 ng/mL, between about 5 ng/mL and about 30 ng/mL, between about 5 ng/mL and about 20 ng/mL, between about 5 ng/mL and about 10 ng/mL, between about 10 ng/mL and about 50 ng/mL, between about 10 ng/mL and about 40 ng/mL, between about 10 ng/mL and about 30 ng/mL, between about 10 ng/mL and about 20 ng/mL, between about 20 ng/mL and about 50 ng/mL, between about 20 ng/mL and about 40 ng/mL, between about 20 ng/mL and about 30 ng/mL, between about 30 ng/mL and about 50 ng/mL, between about 30 ng/mL and about 40 ng/mL, or between about 40 ng/mL and about 50 ng/mL. In particular embodiments, the concentration of recombinant IL-27 during at least a portion of the culturing, e.g. added at the initiation of the culturing and optionally one or more times during the culturing, is between about 1 ng/mL and about 50 ng/mL. In particular embodiments, the concentration of recombinant IL-27 during at least a portion of the culturing, e.g. added at the initiation of the culturing and optionally one or more times during the culturing, is at or about 10 ng/mL.

In some embodiments, the methods include exchanging the culture medium, which, in some aspects includes washing the cells. For example, during at least a portion of the culture or incubation the culture medium can be exchanged or washed out intermittently, such as daily, every other day, every three days, or once a week. In particular embodiments, the culture medium is exchanged or washed out beginning within or within about 3 days to 7 days after initiation of the culture, such as at or about at day 3, day 4, day 5, day 6 or day 7. In particular embodiments, the culture medium is exchanged or washed out at or about beginning at day 5. For example, media is exchanged on day 5 and every 2-3 days afterwards.

Once the culture medium is removed or washed out, it is replenished. In some embodiments, the replenished culture medium includes the one or more growth factors or cytokines, such as any as described above. Hence, in some embodiments, the one or more growth factor or cytokine, such as recombinant IL-2, IL-15 and/or IL-21, is added intermittently during the incubation or culture. In some such aspects, the one or more growth factor or cytokine, such as recombinant IL-2, IL-15 and/or IL-21, is added at or about at the initiation of the culture or incubation, and then is added intermittently during the culture or incubation, such as each time the culture medium is exchanged or washed out. In some embodiments, the one or more growth factor or cytokine, such as recombinant IL-2, IL-15 and/or IL-21, is added to the culture or incubation beginning at day 0 (initiation of the incubation) and, at each exchange or wash out of the culture medium, it is further added to replenish the culture or incubation with the one or more growth factor or cytokine, such as recombinant IL-2, IL-15 and/or IL-21. In some embodiments, the methods include adding the one or more growth factor or cytokine, e.g. recombinant IL-2, IL-15 and/or IL-21, at the initiation of the incubation (day 0), and every two or three days at each wash or exchange of the culture medium for the duration of the incubation, e.g. at or about at day 5, day 7, day 9, day 11, and day 14 of the culture or incubation.

In particular embodiments, the culturing is carried out in the presence of at least one of IL-2, IL-15 and IL-21 and the culture medium is replenished to include at least one of IL-2, IL-15 and IL-21. In some embodiments, the culturing is carried out in the presence of IL-2 and IL-21 and the culture medium is replenished to include IL-2 and IL-21. In some embodiments, the culturing is carried out in the presence of IL-2 and IL-15 and the culture medium is replenished to include IL-2 and IL-15. In some embodiments, the culturing is carried out in the presence of IL-15 and IL-21 and the culture medium is replenished to include IL-15 and IL21. In some embodiments, the culturing is carried out in the presence of IL-2, IL-15 and IL-21 and the culture medium is replenished to include IL-2, IL-15 and IL-21. In some embodiments, one or more additional cytokines can be utilized in the expansion of the NK cells, including but not limited to recombinant IL-18, recombinant IL-7, and/or recombinant IL-12.

In some embodiments, the replenished culture medium includes the one or more growth factors or cytokines, such as recombinant IL-2. Hence, in some embodiments, the growth factor or cytokine, such as recombinant IL-2, is added intermittently during the incubation or culture. In some such aspects, the growth factor or cytokine, such as recombinant IL-2, is added at or about at the initiation of the culture or incubation, and then is added intermittently during the culture or incubation, such as each time the culture medium is exchanged or washed out. In some embodiments, the growth factor or cytokine, such as recombinant IL-2, is added to the culture or incubation beginning at day 0 (initiation of the incubation) and, at each exchange or wash out of the culture medium, it is further added to replenish the culture or incubation with the growth factor or cytokine, such as recombinant IL-2. In some embodiments, the methods include adding recombinant IL-2 at the initiation of the incubation (day 0), and every two or three days at each wash or exchange of the culture medium for the duration of the incubation, e.g. at or about at day 5, day 7, day 9, day 11, and day 14 of the culture or incubation. In any of such embodiments, the recombinant IL-2 is added to the culture or incubation at a concentration of between at or about 1 IU/mL and at or about 500 IU/mL, such as between at or about 1 IU/mL and at or about 250 IU/mL, between at or about 1 IU/mL and at or about 100 IU/mL, between at or about 1 IU/mL and at or about 50 IU/mL, between at or about 50 IU/mL and at or about 500 IU/mL, between at or about 50 IU/mL and at or about 250 IU/mL, between at or about 50 IU/mL and at or about 100 IU/mL, between at or about 100 IU/mL and at or about 500 IU/mL, between at or about 100 IU/mL and at or about 250 IU/mL or between at or about 250 IU/mL and at or about 500 IU/mL, each inclusive. In some embodiments, the recombinant IL-2 is added to the culture or incubation at a concentration that is at or about 50 IU/mL, 60 IU/mL, 70 IU/mL, 80 IU/mL, 90 IU/mL, 100 IU/mL, 125 IU/mL, 150 IU/mL, 200 IU/mL, or any value between any of the foregoing. In particular embodiments, the concentration of the recombinant IL-2 is or is about 100 IU/mL. In particular embodiments, the concentration of the recombinant IL-2 is or is about 500 IU/mL.

In some embodiments, the replenished culture medium includes the one or more growth factors or cytokines, such as recombinant IL-21. Hence, in some embodiments, the growth factor or cytokine, such as recombinant IL-21, is added intermittently during the incubation or culture. In some such aspects, the growth factor or cytokine, such as recombinant IL-21, is added at or about at the initiation of the culture or incubation, and then is added intermittently during the culture or incubation, such as each time the culture medium is exchanged or washed out. In some embodiments, the growth factor or cytokine, such as recombinant IL-21, is added to the culture or incubation beginning at day 0 (initiation of the incubation) and, at each exchange or wash out of the culture medium, it is further added to replenish the culture or incubation with the growth factor or cytokine, such as recombinant IL-21. In some embodiments, the methods include adding recombinant IL-21 at the initiation of the incubation (day 0), and every two or three days at each wash or exchange of the culture medium for the duration of the incubation, e.g. at or about at day 5, day 7, day 9, day 11, and day 14 of the culture or incubation. In any of such embodiments, the recombinant IL-21 is added to the culture or incubation at a concentration of between about 10 ng/mL and about 100 ng/mL, between about 10 ng/mL and about 90 ng/mL, between about 10 ng/mL and about 80 ng/mL, between about 10 ng/mL and about 70 ng/mL, between about 10 ng/mL and about 60 ng/mL, between about 10 ng/mL and about 50 ng/mL, between about 10 ng/mL and about 40 ng/mL, between about 10 ng/mL and about 30 ng/mL, between about 10 ng/mL and about 20 ng/mL, between about 20 ng/mL and about 100 ng/mL, between about 20 ng/mL and about 90 ng/mL, between about 20 ng/mL and about 80 ng/mL, between about 20 ng/mL and about 70 ng/mL, between about 20 ng/mL and about 60 ng/mL, between about 20 ng/mL and about 50 ng/mL, between about 20 ng/mL and about 40 ng/mL, between about 20 ng/mL and about 30 ng/mL, between about 30 ng/mL and about 100 ng/mL, between about 30 ng/mL and about 90 ng/mL, between about 30 ng/mL and about 80 ng/mL, between about 30 ng/mL and about 70 ng/mL, between about 30 ng/mL and about 60 ng/mL, between about 30 ng/mL and about 50 ng/mL, between about 30 ng/mL and about 40 ng/mL, between about 40 ng/mL and about 100 ng/mL, between about 40 ng/mL and about 90 ng/mL, between about 40 ng/mL and about 80 ng/mL, between about 40 ng/mL and about 70 ng/mL, between about 40 ng/mL and about 60 ng/mL, between about 40 ng/mL and about 50 ng/mL, between about 50 ng/mL and about 100 ng/mL, between about 50 ng/mL and about 90 ng/mL, between about 50 ng/mL and about 80 ng/mL, between about 50 ng/mL and about 70 ng/mL, between about 50 ng/mL and about 60 ng/mL, between about 60 ng/mL and about 100 ng/mL, between about 60 ng/mL and about 90 ng/mL, between about 60 ng/mL and about 80 ng/mL, between about 60 ng/mL and about 70 ng/mL, between about 70 ng/mL and about 100 ng/mL, between about 70 ng/mL and about 90 ng/mL, between about 70 ng/mL and about 80 ng/mL, between about 80 ng/mL and about 100 ng/mL, between about 80 ng/mL and about 90 ng/mL, or between about 90 ng/mL and about 100 ng/mL, inclusive. In particular embodiments, the recombinant IL-21 is added to the culture or incubation at a concentration of between about 10 ng/mL and about 100 ng/mL, inclusive. The recombinant IL-21 is added to the culture or incubation at a concentration of at or about 25 ng/mL.

In some embodiments, the replenished culture medium includes the one or more growth factors or cytokines, such as recombinant IL-21, added as a complex with an antibody, such as an anti-IL-21 antibody. Hence, in some embodiments, the complex, such as an IL-21/anti-IL-21 antibody complex, is added intermittently during the incubation or culture. In some such aspects, the complex, such as an IL-21/anti-IL-21 antibody complex, is added at or about at the initiation of the culture or incubation, and then is added intermittently during the culture or incubation, such as each time the culture medium is exchanged or washed out. In some embodiments, the complex, such as an IL-21/anti-IL-21 antibody complex, is added to the culture or incubation beginning at day 0 (initiation of the incubation) and, at each exchange or wash out of the culture medium, it is further added to replenish the culture or incubation with the complex, such as an IL-21/anti-IL-21 antibody complex. In some embodiments, the methods include adding the complex, such as an IL-21/anti-IL-21 antibody complex, at the initiation of the incubation (day 0), and every two or three days at each wash or exchange of the culture medium for the duration of the incubation, e.g. at or about at day 5, day 7, day 9, day 11, and day 14 of the culture or incubation. In any of such embodiments, the anti-IL-21 antibody is contacted with the recombinant IL-21, thereby forming an IL-21/anti-IL-21 complex, and the IL-21/anti-IL-21 complex is added to the culture medium. In any of such embodiments, contacting the recombinant IL-21 and the anti-IL-21 antibody to form an IL-21/anti-IL-21 complex is carried out under conditions that include temperature and time suitable for the formation of the complex. In any of such embodiments, the culturing is carried out at 37° C.±2 for 30 minutes. In any of such embodiments, anti-IL-21 antibody is added at a concentration between at or about 100 ng/mL and at or about 500 ng/mL, between at or about 100 ng/mL and at or about 400 ng/mL, between at or about 100 ng/mL and at or about 300 ng/mL, between at or about 100 ng/mL and at or about 200 ng/mL, between at or about 200 ng/mL and at or about 500 ng/mL, between at or about 200 ng/mL and at or about 400 ng/mL, between at or about 200 ng/mL and at or about 300 ng/mL, between at or about 300 ng/mL and at or about 500 ng/mL, between at or about 300 ng/mL and at or about 400 ng/mL, or between at or about 400 ng/mL and at or about 500 ng/mL. In some embodiments, anti-IL-21 antibody is added at a concentration between at or about 100 ng/mL and at or about 500 ng/mL. In some embodiments, anti-IL-21 antibody is added at a concentration of 250 ng/mL. In any of such embodiments, the concentration of recombinant IL-21 used to form a complex with the anti-IL-21 antibody is between about 10 ng/mL and about 100 ng/mL, between about 10 ng/mL and about 90 ng/mL, between about 10 ng/mL and about 80 ng/mL, between about 10 ng/mL and about 70 ng/mL, between about 10 ng/mL and about 60 ng/mL, between about 10 ng/mL and about 50 ng/mL, between about 10 ng/mL and about 40 ng/mL, between about 10 ng/mL and about 30 ng/mL, between about 10 ng/mL and about 20 ng/mL, between about 20 ng/mL and about 100 ng/mL, between about 20 ng/mL and about 90 ng/mL, between about 20 ng/mL and about 80 ng/mL, between about 20 ng/mL and about 70 ng/mL, between about 20 ng/mL and about 60 ng/mL, between about 20 ng/mL and about 50 ng/mL, between about 20 ng/mL and about 40 ng/mL, between about 20 ng/mL and about 30 ng/mL, between about 30 ng/mL and about 100 ng/mL, between about 30 ng/mL and about 90 ng/mL, between about 30 ng/mL and about 80 ng/mL, between about 30 ng/mL and about 70 ng/mL, between about 30 ng/mL and about 60 ng/mL, between about 30 ng/mL and about 50 ng/mL, between about 30 ng/mL and about 40 ng/mL, between about 40 ng/mL and about 100 ng/mL, between about 40 ng/mL and about 90 ng/mL, between about 40 ng/mL and about 80 ng/mL, between about 40 ng/mL and about 70 ng/mL, between about 40 ng/mL and about 60 ng/mL, between about 40 ng/mL and about 50 ng/mL, between about 50 ng/mL and about 100 ng/mL, between about 50 ng/mL and about 90 ng/mL, between about 50 ng/mL and about 80 ng/mL, between about 50 ng/mL and about 70 ng/mL, between about 50 ng/mL and about 60 ng/mL, between about 60 ng/mL and about 100 ng/mL, between about 60 ng/mL and about 90 ng/mL, between about 60 ng/mL and about 80 ng/mL, between about 60 ng/mL and about 70 ng/mL, between about 70 ng/mL and about 100 ng/mL, between about 70 ng/mL and about 90 ng/mL, between about 70 ng/mL and about 80 ng/mL, between about 80 ng/mL and about 100 ng/mL, between about 80 ng/mL and about 90 ng/mL, or between about 90 ng/mL and about 100 ng/mL, inclusive. In particular embodiments, the concentration of recombinant IL-21 used to form a complex with the anti-IL-21 antibody is between about 10 ng/mL and about 100 ng/mL, inclusive. In particular embodiments, the concentration of recombinant IL-21 used to form a complex with the anti-IL-21 antibody is at or about 25 ng/mL.

In some embodiments, the replenished culture medium includes the one or more growth factors or cytokines, such as recombinant IL-15. Hence, in some embodiments, the growth factor or cytokine, such as recombinant IL-15, is added intermittently during the incubation or culture. In some such aspects, the growth factor or cytokine, such as recombinant IL-15, is added at or about at the initiation of the culture or incubation, and then is added intermittently during the culture or incubation, such as each time the culture medium is exchanged or washed out. In some embodiments, the growth factor or cytokine, such as recombinant IL-15, is added to the culture or incubation beginning at day 0 (initiation of the incubation) and, at each exchange or wash out of the culture medium, it is further added to replenish the culture or incubation with the growth factor or cytokine, such as recombinant IL-15. In some embodiments, the methods include adding recombinant IL-15 at the initiation of the incubation (day 0), and every two or three days at each wash or exchange of the culture medium for the duration of the incubation, e.g. at or about at day 5, day 7, day 9, day 11, and day 14 of the culture or incubation. In any of such embodiments, the recombinant IL-15 is added to the culture or incubation at a concentration of between about 1 ng/mL and about 50 ng/mL, between about 1 ng/mL and about 40 ng/mL, between about 1 ng/mL and about 30 ng/mL, between about 1 ng/mL and about 20 ng/mL, between about 1 ng/mL and about 10 ng/mL, between about 1 ng/mL and about 5 ng/mL, between about 5 ng/mL and about 50 ng/mL, between about 5 ng/mL and about 40 ng/mL, between about 5 ng/mL and about 30 ng/mL, between about 5 ng/mL and about 20 ng/mL, between about 5 ng/mL and about 10 ng/mL, between about 10 ng/mL and about 50 ng/mL, between about 10 ng/mL and about 40 ng/mL, between about 10 ng/mL and about 30 ng/mL, between about 10 ng/mL and about 20 ng/mL, between about 20 ng/mL and about 50 ng/mL, between about 20 ng/mL and about 40 ng/mL, between about 20 ng/mL and about 30 ng/mL, between about 30 ng/mL and about 50 ng/mL, between about 30 ng/mL and about 40 ng/mL, or between about 40 ng/mL and about 50 ng/mL. In any of such embodiments, the recombinant IL-15 is added to the culture or incubation at a concentration of between about 1 ng/mL and about 50 ng/mL. In any of such embodiments, the recombinant IL-15 is added to the culture or incubation at a concentration of at or about 10 ng/mL. In particular embodiments, 500 IU/mL of IL-2, 10 ng/mL of IL-15, and 25 ng/mL of IL-21 are added to the culture or incubation.

In some embodiments, the replenished culture medium includes the one or more growth factors or cytokines, such as recombinant IL-12. Hence, in some embodiments, the growth factor or cytokine, such as recombinant IL-12, is added intermittently during the incubation or culture. In some such aspects, the growth factor or cytokine, such as recombinant IL-12, is added at or about at the initiation of the culture or incubation, and then is added intermittently during the culture or incubation, such as each time the culture medium is exchanged or washed out. In some embodiments, the growth factor or cytokine, such as recombinant IL-12, is added to the culture or incubation beginning at day 0 (initiation of the incubation) and, at each exchange or wash out of the culture medium, it is further added to replenish the culture or incubation with the growth factor or cytokine, such as recombinant IL-12. In some embodiments, the methods include adding recombinant IL-12 at the initiation of the incubation (day 0), and every two or three days at each wash or exchange of the culture medium for the duration of the incubation, e.g. at or about at day 5, day 7, day 9, day 11, and day 14 of the culture or incubation. In any of such embodiments, the recombinant IL-12 is added to the culture or incubation at a concentration of between about 1 ng/mL and about 50 ng/mL, between about 1 ng/mL and about 40 ng/mL, between about 1 ng/mL and about 30 ng/mL, between about 1 ng/mL and about 20 ng/mL, between about 1 ng/mL and about 10 ng/mL, between about 1 ng/mL and about 5 ng/mL, between about 5 ng/mL and about 50 ng/mL, between about 5 ng/mL and about 40 ng/mL, between about 5 ng/mL and about 30 ng/mL, between about 5 ng/mL and about 20 ng/mL, between about 5 ng/mL and about 10 ng/mL, between about 10 ng/mL and about 50 ng/mL, between about 10 ng/mL and about 40 ng/mL, between about 10 ng/mL and about 30 ng/mL, between about 10 ng/mL and about 20 ng/mL, between about 20 ng/mL and about 50 ng/mL, between about 20 ng/mL and about 40 ng/mL, between about 20 ng/mL and about 30 ng/mL, between about 30 ng/mL and about 50 ng/mL, between about 30 ng/mL and about 40 ng/mL, or between about 40 ng/mL and about 50 ng/mL. In any of such embodiments, the recombinant IL-12 is added to the culture or incubation at a concentration of between about 1 ng/mL and about 50 ng/mL. In any of such embodiments, the recombinant IL-12 is added to the culture or incubation at a concentration of at or about 10 ng/mL.

In some embodiments, the replenished culture medium includes the one or more growth factors or cytokines, such as recombinant IL-18. Hence, in some embodiments, the growth factor or cytokine, such as recombinant IL-18, is added intermittently during the incubation or culture. In some such aspects, the growth factor or cytokine, such as recombinant IL-18, is added at or about at the initiation of the culture or incubation, and then is added intermittently during the culture or incubation, such as each time the culture medium is exchanged or washed out. In some embodiments, the growth factor or cytokine, such as recombinant IL-18, is added to the culture or incubation beginning at day 0 (initiation of the incubation) and, at each exchange or wash out of the culture medium, it is further added to replenish the culture or incubation with the growth factor or cytokine, such as recombinant IL-18. In some embodiments, the methods include adding recombinant IL-18 at the initiation of the incubation (day 0), and every two or three days at each wash or exchange of the culture medium for the duration of the incubation, e.g. at or about at day 5, day 7, day 9, day 11, and day 14 of the culture or incubation. In any of such embodiments, the recombinant IL-18 is added to the culture or incubation at a concentration of between about 1 ng/mL and about 50 ng/mL, between about 1 ng/mL and about 40 ng/mL, between about 1 ng/mL and about 30 ng/mL, between about 1 ng/mL and about 20 ng/mL, between about 1 ng/mL and about 10 ng/mL, between about 1 ng/mL and about 5 ng/mL, between about 5 ng/mL and about 50 ng/mL, between about 5 ng/mL and about 40 ng/mL, between about 5 ng/mL and about 30 ng/mL, between about 5 ng/mL and about 20 ng/mL, between about 5 ng/mL and about 10 ng/mL, between about 10 ng/mL and about 50 ng/mL, between about 10 ng/mL and about 40 ng/mL, between about 10 ng/mL and about 30 ng/mL, between about 10 ng/mL and about 20 ng/mL, between about 20 ng/mL and about 50 ng/mL, between about 20 ng/mL and about 40 ng/mL, between about 20 ng/mL and about 30 ng/mL, between about 30 ng/mL and about 50 ng/mL, between about 30 ng/mL and about 40 ng/mL, or between about 40 ng/mL and about 50 ng/mL. In any of such embodiments, the recombinant IL-18 is added to the culture or incubation at a concentration of between about 1 ng/mL and about 50 ng/mL. In any of such embodiments, the recombinant IL-18 is added to the culture or incubation at a concentration of at or about 10 ng/mL.

In some embodiments, the replenished culture medium includes the one or more growth factors or cytokines, such as recombinant IL-27. Hence, in some embodiments, the growth factor or cytokine, such as recombinant IL-27, is added intermittently during the incubation or culture. In some such aspects, the growth factor or cytokine, such as recombinant IL-27, is added at or about at the initiation of the culture or incubation, and then is added intermittently during the culture or incubation, such as each time the culture medium is exchanged or washed out. In some embodiments, the growth factor or cytokine, such as recombinant IL-27, is added to the culture or incubation beginning at day 0 (initiation of the incubation) and, at each exchange or wash out of the culture medium, it is further added to replenish the culture or incubation with the growth factor or cytokine, such as recombinant IL-27. In some embodiments, the methods include adding recombinant IL-27 at the initiation of the incubation (day 0), and every two or three days at each wash or exchange of the culture medium for the duration of the incubation, e.g. at or about at day 5, day 7, day 9, day 11, and day 14 of the culture or incubation. In any of such embodiments, the recombinant IL-27 is added to the culture or incubation at a concentration of between about 1 ng/mL and about 50 ng/mL, between about 1 ng/mL and about 40 ng/mL, between about 1 ng/mL and about 30 ng/mL, between about 1 ng/mL and about 20 ng/mL, between about 1 ng/mL and about 10 ng/mL, between about 1 ng/mL and about 5 ng/mL, between about 5 ng/mL and about 50 ng/mL, between about 5 ng/mL and about 40 ng/mL, between about 5 ng/mL and about 30 ng/mL, between about 5 ng/mL and about 20 ng/mL, between about 5 ng/mL and about 10 ng/mL, between about 10 ng/mL and about 50 ng/mL, between about 10 ng/mL and about 40 ng/mL, between about 10 ng/mL and about 30 ng/mL, between about 10 ng/mL and about 20 ng/mL, between about 20 ng/mL and about 50 ng/mL, between about 20 ng/mL and about 40 ng/mL, between about 20 ng/mL and about 30 ng/mL, between about 30 ng/mL and about 50 ng/mL, between about 30 ng/mL and about 40 ng/mL, or between about 40 ng/mL and about 50 ng/mL. In any of such embodiments, the recombinant IL-27 is added to the culture or incubation at a concentration of between about 1 ng/mL and about 50 ng/mL. In any of such embodiments, the recombinant IL-27 is added to the culture or incubation at a concentration of at or about 10 ng/mL.

In embodiments of the provided methods, culturing or incubating includes providing the chemical and physical conditions (e.g., temperature, gas) which are required or useful for NK cell maintenance. Examples of chemical conditions which may support NK cell proliferation or expansion include but are not limited to buffers, nutrients, serum, vitamins and antibiotics which are typically provided in the growth (i.e., culture) medium. In one embodiment, the NK culture medium includes MEMα comprising 10% FCS or CellGro SCGM (Cell Genix) comprising 5% Human Serum/LiforCell® FBS Replacement (Lifeblood Products). Other media suitable for use with the invention include, but are not limited to Glascow's medium (Gibco Carlsbad Calif.), RPMI medium (Sigma-Aldrich, St Louis Mo.) or DMEM (Sigma-Aldrich, St Louis Mo.). It will be noted that many of the culture media contain nicotinamide as a vitamin supplement for example, MEMα (8.19 μM nicotinamide), RPMI (8.19 μM nicotinamide), DMEM (32.78 μM nicotinamide) and Glascow's medium (16.39 μM nicotinamide).

In some embodiments, such as for applications in which cells are introduced (or reintroduced) into a human subject, culturing is carried out using serum-free formulations, such as AIM V™ serum free medium for lymphocyte culture, MARROWMAX™ bone marrow medium or serum-free stem cell growth medium (SCGM) (e.g. CellGenix® GMP SCGM). Such medium formulations and supplements are available from commercial sources. The cultures can be supplemented with amino acids, antibiotics, and/or with other growth factors cytokines as described to promote optimal viability, proliferation, functionality and/or and survival. In some embodiments, the serum-free media also may be supplemented with a low percentage of human serum, such as 0.5% to 10% human serum, such as at or about 5% human serum. In such embodiments, the human serum can be human serum from human AB plasma (human AB serum) or autologous serum.

In some embodiments, the culturing with feeder cells, and optionally cytokines (e.g. recombinant IL-2 or IL-21) is carried out under conditions that include temperature suitable for the growth or expansion of human NK cells, for example, at least about 25 degrees Celsius, generally at least about 30 degrees, and generally at or about 37 degrees Celsius. In some embodiments, the culturing is carried out at 37° C.±2 in 5% CO₂.

In embodiments of the provided methods, the culturing includes incubation that is carried out under GMP conditions. In some embodiments, the incubation is in a closed system, which in some aspects may be a closed automated system. In some embodiments, the culture media containing the one or more recombinant cytokines or growth factors is a serum-free media. In some embodiments, the incubation is carried out in a closed automated system and with serum-free media.

In some embodiments, the expansion of the NK cells is carried out in a culture vessel suitable for cell expansion. In some embodiments, the culture vessel is a gas permeable culture vessel, such as a G-Rex system (e.g. G-Rex 10, G-Rex 10M, G-Rex 100 M/100M-CS or G-Rex 500 M/500M-CS). In some embodiments the culture vessel is a microplate, flask, bag or other culture vessel suitable for expansion of cells in a closed system. In some embodiments, expansion can be carried out in a bioreactor. In some embodiments, the expansion is carried out using a cell expansion system by transfer of the cells to gas permeable bags, such as in connection with a bioreactor (e.g. Xuri Cell Expansion System W25 (GE Healthcare)). In an embodiment, the cell expansion system includes a culture vessel, such as a bag, e.g. gas permeable cell bag, with a volume that is about 50 mL, about 100 mL, about 200 mL, about 300 mL, about 400 mL, about 500 mL, about 600 mL, about 700 mL, about 800 mL, about 900 mL, about 1 L, about 2 L, about 3 L, about 4 L, about 5 L, about 6 L, about 7 L, about 8 L, about 9 L, and about 10 L, or any value between any of the foregoing. In some embodiments, the process is automated or semi-automated. In some aspects, the expansion culture is carried out under static conditions. In some embodiments, the expansion culture is carried out under rocking conditions. The medium can be added in bolus or can be added on a perfusion schedule. In some embodiments, the bioreactor maintains the temperature at or near 37° C. and CO2 levels at or near 5% with a steady air flow at, at about, or at least 0.01 L/min, 0.05 L/min, 0.1 L/min, 0.2 L/min, 0.3 L/min, 0.4 L/min, 0.5 L/min, 1.0 L/min, 1.5 L/min, or 2.0 L/min or greater than 2.0 L/min. In certain embodiments, at least a portion of the culturing is performed with perfusion, such as with a rate of 290 ml/day, 580 ml/day, and/or 1160 ml/day.

In some aspects, cells are expanded in an automated closed expansion system that is perfusion enabled. Perfusions can continuously add media to the cells to ensure an optimal growth rate is achieved.

The expansion methods can be carried out under GMP conditions, including in a closed automated system and using serum free medium. In some embodiments, any one or more of the steps of the method can be carried out in a closed system or under GMP conditions. In certain embodiments, all process operations are performed in a GMP suite. In some embodiments, a closed system is used for carrying out one or more of the other processing steps of a method for manufacturing, generating or producing a cell therapy. In some embodiments, one or more or all of the processing steps, e.g., isolation, selection and/or enrichment, processing, culturing steps including incubation in connection with expansion of the cells, and formulation steps is carried out using a system, device, or apparatus in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some of any of the provided embodiments, the culturing is carried out until a time at which the method achieves expansion of at least or at least about 2.50×10⁸ g-NK cells. In some of any of the provided embodiments, the culturing is carried out until a time at which the method achieves expansion of at least or at least about 5.0×10⁸ g-NK cells. In some of any of the provided embodiments, the culturing is carried out until the method achieves expansion of at least or at least about 1.0×10⁹ g-NK cells. In some of any of the provided embodiments, the culturing is carried out until a time at which the method achieves expansion of at least or at least about 5.0×10⁹ g-NK cells.

In some of any of the provided embodiments, the culturing is carried out for at or about or at least at or at least about 5 days, 6 days, 7 days, 8 days, 9 days, 10 days, 11 days, 12 days, 13 days, 14 days, 15 days, 16 days, 17 days, 18 days, 19 days, 20 day, 21 days, 22 days, 23 days, 24 days or 25 days. In some embodiments, the culturing is carried out for at or about or at least at or about 14 days. In some embodiments the culturing is carried out for at or about or at least at or about 21 days.

In some of any of the provided embodiments, the culturing or incubation in accord with any of the provided methods is carried out for at or about or at least at or at least about 5 days, 6 days, 7 days, 8 days, 9 days, 10 days, 11 days, 12 days, 13 days, 14 days, 15 days, 16 days, 17 days, 18 days, 19 days, 20 day, 21 days, 22 days, 23 days, 24 days or 25 days. In some embodiments, the culturing is carried out for at or about or at least at or about 14 days. In some embodiments, the culturing is carried out for at or about or at least at or about 21 days. In certain embodiments, a longer duration of culturing is typically necessary if the enriched NK cells at the initiation of the culturing have been thawed after having been previously frozen or cryopreserved. It is within the level of a skilled artisan to empirically determine the optimal number of days to culture the cells depending on factors such as the state of the cells at the initiation of the culture, the health or viability of the cells that the initiation of the culture or during the culturing and/or the desired number of threshold cells at the end of the culturing depending, for example, on the desired application of the cells, such as the dose of cells to be administered to a subject for therapeutic purposes.

At the end of the culturing, the cells are harvested. Collection or harvesting of the cells can be achieved by centrifugation of the cells from the culture vessel after the end of the culturing. For example, cells are harvested by centrifugation after approximately 14 days of culture. After harvesting of the cells, the cells are washed. A sample of the cells can be collected for functional or phenotypic testing. Any other cells not used for functional or phenotypic testing can be separately formulated. In some cases, the cells are formulated with a cryoprotectant for cryopreservation of cells.

In some embodiments, the provided methods include steps for freezing, e.g., cryopreserving, the cells, either before or after isolation, selection and/or enrichment. In some embodiments, the provided methods include steps for freezing, e.g., cryopreserving, the cells, either before or after incubation and/or culturing. In some embodiments, the method includes cryopreserving the cells in the presence of a cryoprotectant, thereby producing a cryopreserved composition. In some aspects, prior to the incubating and/or prior to administering to a subject, the method includes washing the cryopreserved composition under conditions to reduce or remove the cryoprotectant. Any of a variety of known freezing solutions and parameters in some aspects may be used. In some embodiments, the cells are frozen, e.g., cryofrozen or cryopreserved, in media and/or solution with a final concentration of or of about 12.5%, 12.0%, 11.5%, 11.0%, 10.5%, 10.0%, 9.5%, 9.0%, 8.5%, 8.0%, 7.5%, 7.0%, 6.5%, 6.0%, 5.5%, or 5.0% DMSO, or between 1% and 15%, between 6% and 12%, between 5% and 10%, or between 6% and 8% DMSO. In particular embodiments, the cells are frozen, e.g., cryofrozen or cryopreserved, in media and/or solution with a final concentration of or of about 5.0%, 4.5%, 4.0%, 3.5%, 3.0%, 2.5%, 2.0%, 1.5%, 1.25%, 1.0%, 0.75%, 0.5%, or 0.25% HSA, or between 0.1% and −5%, between 0.25% and 4%, between 0.5% and 2%, or between 1% and 2% HSA. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to or to about −80° C. at a rate of or of about 1° per minute and stored in the vapor phase of a liquid nitrogen storage tank. In some embodiments, the cells are frozen in a serum-free cryopreservation medium comprising a cryoprotectant. In some embodiments, the cryoprotectant is DMSO. In some embodiments, the cryopreservation medium is between at or about 5% and at or about 10% DMSO (v/v). In some embodiments, the cryopreservation medium is at or about 5% DMSO (v/v). In some embodiments, the cryopreservation medium is at or about 6% DMSO (v/v). In some embodiments, the cryopreservation medium is at or about 7% DMSO (v/v). In some embodiments, the cryopreservation medium is at or about 8% DMSO (v/v). In some embodiments, the cryopreservation medium is at or about 9% DMSO (v/v). In some embodiments, the cryopreservation medium is at or about 10% DMSO (v/v). In some embodiments, the cryopreservation medium contains a commercially available cryopreservation solution (CryoStor™ CS10 or CS5). CryoStor™ CS10 is a cryopreservation medium containing 10% dimethyl sulfoxide (DMSO). CryoStor™ CSS is a cryopreservation medium containing 5% dimethyl sulfoxide (DMSO). In some embodiments, the cryopreservation media contains one or more additional excipients, such as plasmalyte A or human serum albumin (HSA).

In some embodiments, the cells are cryopreserved at a density of 5×10⁶ to 1×10⁸ cells/mL. For example, the cells are cryopreserved at a density of at or about 5×10⁶ cells/mL, at or about 10×10⁶ cells/mL, at or about 15×10⁶ cells/mL, at or about 20×10⁶ cells/mL, at or about 25×10⁶ cells/mL, at or about 30×10⁶ cells/mL, at or about 40×10⁶ cells/mL, at or about 50×10⁶ cells/mL, at or about 60×10⁶ cells/mL, at or about 70×10⁶ cells/mL, at or about 80×10⁶ cells/mL or at or about 90×10⁶ cells/mL, or any value between any of the foregoing. The cells can be cryopreserved in any volume as suitable for the cryopreservation vessel. In some embodiments, the cells are cryopreserved in a vial. The volume of the cryopreservation media may be between at or about 1 mL and at or about 50 mL, such as at or about 1 mL and 5 mL. In some embodiments, the cells are cryopreserved in a bag. The volume of the cryopreservation media may between at or about 10 mL and at or about 500 mL, such as between at or about 100 mL or at or about 200 mL. The harvested and expanded cells can be cryopreserved at low temperature environments, such as temperatures of −80° C. to −196° C. In some of any of the provided methods, the method produces an increased number of NKG2C^{pos} cells at the end of the culturing compared to at the initiation of the culturing. For example, the increase in NKG2C^{pos} cells at the end of culturing compared to at the initiation of the culturing can be greater than or greater than about 100-fold, greater than or greater than about 200-fold, greater than or greater than about 300-fold, greater than or greater than about 400-fold, greater than or greater than about 500-fold, greater than or greater than about 600-fold, greater than or greater than about 700-fold or greater than or greater than about 800-fold. In some of any embodiments, the increase is at or about 1000-fold greater. In some of any embodiments, the increase is at or about 2000-fold greater. In some of any embodiments, the increase is at or about 2500-fold greater. In some of any embodiments, the increase is at or about 3000-fold greater. In some of any embodiments, the increase is at or about 5000-fold greater. In some of any embodiments, the increase is at or about 10000-fold greater. In some of any embodiments, the increase is at or about 15000-fold greater. In some of any embodiments, the increase is at or about 20000-fold greater. In some of any embodiments, the increase is at or about 25000-fold greater. In some of any embodiments, the increase is at or about 30000-fold greater. In some of any embodiments, the increase is at or about 35000-fold greater. In some embodiments, the culturing or incubation in accord with any of the provided methods is carried out until a time at which the method achieves expansion of at least at or about 2.50×10⁸ NKG2C^{pos} cells, at least at or about 3.0×10⁸ NKG2C^{pos} cells, at least at or about 4.0×10⁸ NKG2C^{pos} cells, at least at or about 5.0×10⁸ NKG2C^{pos} cells, at least at or about 6.0×10⁸ NKG2C^{pos} cells, at least at or about 7.0×10⁸ NKG2C^{pos} cells, at least at or about 8.0×10⁸ NKG2C^{pos} cells, at least at or about 9.0×10⁸ NKG2C^{pos} cells, at least at or about 1.0×10⁹ NKG2C^{pos} cells, at least at or about 1.5×10⁹ NKG2C^{pos} cells, at least at or about 2.0×10⁹ NKG2C^{pos} cells, at least at or about 3.0×10⁹ NKG2C^{pos} cells, at least at or about 4.0×10⁹ NKG2C^{pos} cells, at least at or about 5.0×10⁹ NKG2C^{pos} cells, at least at or about 1.0×10¹⁰ NKG2C^{pos} cells, at least at or about 1.5×10¹⁰ NKG2C^{pos} cells, at least at or about 2.0×10¹⁰ NKG2C^{pos} cells, at least at or about 2.5×10¹⁰ NKG2C^{pos} cells or more.

In some of any of the provided methods, the method produces an increased number of NKG2A^{neg} cells at the end of the culturing compared to at the initiation of the culturing. For example, the increase in NKG2A^{neg} cells at the end of culturing compared to at the initiation of the culturing can be greater than or greater than about 100-fold, greater than or greater than about 200-fold, greater than or greater than about 300-fold, greater than or greater than about 400-fold, greater than or greater than about 500-fold, greater than or greater than about 600-fold, greater than or greater than about 700-fold or greater than or greater than about 800-fold. In some of any embodiments, the increase is at or about 1000-fold greater. In some of any embodiments, the increase is at or about 2000-fold greater. In some of any embodiments, the increase is at or about 3000-fold greater. In some of any embodiments, the increase is at or about 2500-fold greater. In some of any embodiments, the increase is at or about 5000-fold greater. In some of any embodiments, the increase is at or about 10000-fold greater. In some of any embodiments, the increase is at or about 15000-fold greater. In some of any embodiments, the increase is at or about 20000-fold greater. In some of any embodiments, the increase is at or about 25000-fold greater. In some of any embodiments, the increase is at or about 30000-fold greater. In some of any embodiments, the increase is at or about 35000-fold greater. In some embodiments, the culturing or incubation in accord with any of the provided methods is carried out until a time at which the method achieves expansion of at least at or about 2.50×10⁸ NKG2A^{neg} cells, at least at or about 3.0×10⁸ NKG2A^{neg} cells, at least at or about 4.0×10⁸ NKG2A^{neg} cells, at least at or about 5.0×10⁸ NKG2A^{neg} cells, at least at or about 6.0×10⁸ NKG2A^{neg} cells, at least at or about 7.0×10⁸ NKG2A^{neg} cells, at least at or about 8.0×10⁸ NKG2A^{neg} cells, at least at or about 9.0×10⁸ NKG2A^{neg} cells, at least at or about 1.0×10⁹ NKG2A^{neg} cells, at least at or about 1.5×10⁹ NKG2A^{neg} cells, at least at or about 2.0×10⁹ NKG2A^{neg} cells, at least at or about 3.0×10⁹ NKG2A^{neg} cells, at least at or about 4.0×10⁹ NKG2A^{neg} cells, at least at or about 5.0×10⁹ NKG2A^{neg} cells, at least at or about 1.0×10¹⁰ NKG2A^{neg} cells, at least at or about 1.5×10¹⁰ NKG2A^{neg} cells, at least at or about 2.0×10¹⁰ NKG2A^{neg} cells, at least at or about 2.5×10¹⁰ NKG2A^{neg} cells or more.

In some of any of the provided methods, the method produces an increased number of NKG2C^{pos}NKG2A^{neg} cells at the end of the culturing compared to at the initiation of the culturing. For example, the increase in NKG2C^{pos}NKG2A^{neg} cells at the end of culturing compared to at the initiation of the culturing can be greater than or greater than about 100-fold, greater than or greater than about 200-fold, greater than or greater than about 300-fold, greater than or greater than about 400-fold, greater than or greater than about 500-fold, greater than or greater than about 600-fold, greater than or greater than about 700-fold or greater than or greater than about 800-fold. In some of any embodiments, the increase is at or about 1000-fold greater. In some of any embodiments, the increase is at or about 2000-fold greater. In some of any embodiments, the increase is at or about 2500-fold greater. In some of any embodiments, the increase is at or about 3000-fold greater. In some of any embodiments, the increase is at or about 5000-fold greater. In some of any embodiments, the increase is at or about 10000-fold greater. In some of any embodiments, the increase is at or about 15000-fold greater. In some of any embodiments, the increase is at or about 20000-fold greater. In some of any embodiments, the increase is at or about 25000-fold greater. In some of any embodiments, the increase is at or about 30000-fold greater. In some of any embodiments, the increase is at or about 35000-fold greater. In some embodiments, the culturing or incubation in accord with any of the provided methods is carried out until a time at which the method achieves expansion of at least at or about 2.50×10⁸ NKG2C^{pos}NKG2A^{neg} cells, at least at or about 3.0×10⁸ NKG2C^{pos}NKG2A^{neg} cells, at least at or about 4.0×10⁸ NKG2C^{pos}NKG2A^{neg} cells, at least at or about 5.0×10⁸ NKG2C^{pos}NKG2A^{neg} cells, at least at or about 6.0×10⁸ NKG2C^{pos}NKG2A^{neg} cells, at least at or about 7.0×10⁸ NKG2C^{pos}NKG2A^{neg} cells, at least at or about 8.0×10⁸ NKG2C^{pos}NKG2A^{neg} cells, at least at or about 9.0×10⁸ NKG2C^{pos}NKG2A^{neg} cells, at least at or about 1.0×10⁸ NKG2C^{pos}NKG2A^{neg} cells, at least at or about 1.5×10⁹ NKG2C^{pos}NKG2A^{neg} cells, at least at or about 2.0×10⁹ NKG2C^{pos}NKG2A^{neg} cells, at least at or about 3.0×10⁹ NKG2C^{pos}NKG2A^{neg} cells, at least at or about 4.0×10⁹ NKG2C^{pos}NKG2A^{neg} cells, at least at or about 5.0×10⁹ NKG2C^{pos}NKG2A^{neg} cells, at least at or about 1.0×10¹⁰ NKG2C^{pos}NKG2A^{neg} cells, at least at or about 1.5×10¹⁰ NKG2C^{pos}NKG2A^{neg} cells, at least at or about 2.0×10¹⁰ NKG2C^{pos}NKG2A^{neg} cells, at least at or about 2.5×10¹⁰ NKG2C^{pos}NKG2A^{neg} cells or more.

In some of any of the provided methods, the method produces an increased number of g-NK cells at the end of the culturing compared to at the initiation of the culturing. For example, the increase in g-NK cells at the end of culturing compared to at the initiation of the culturing can be greater than or greater than about 100-fold, greater than or greater than about 200-fold, greater than or greater than about 300-fold, greater than or greater than about 400-fold, greater than or greater than about 500-fold, greater than or greater than about 600-fold, greater than or greater than about 700-fold or greater than or greater than about 800-fold. In some of any embodiments, the increase is at or about 1000-fold greater. In some of any embodiments, the increase is at or about 2000-fold greater. In some of any embodiments, the increase is at or about 2500-fold greater. In some of any embodiments, the increase is at or about 3000-fold greater. In some of any embodiments, the increase is at or about 5000-fold greater. In some of any embodiments, the increase is at or about 10000-fold greater. In some of any embodiments, the increase is at or about 15000-fold greater. In some of any embodiments, the increase is at or about 20000-fold greater. In some of any embodiments, the increase is at or about 25000-fold greater. In some of any embodiments, the increase is at or about 30000-fold greater. In some of any embodiments, the increase is at or about 35000-fold greater. In some embodiments, the culturing or incubation in accord with any of the provided methods is carried out until a time at which the method achieves expansion of at least at or about 2.50×10⁸ g-NK cells, at least at or about 3.0×10⁸ g-NK cells, at least at or about 4.0×10⁸ g-NK cells, at least at or about 5.0×10⁸ g-NK cells, at least at or about 6.0×10⁸ g-NK cells, at least at or about 7.0×10⁸ g-NK cells, at least at or about 8.0×10⁸ g-NK cells, at least at or about 9.0×10⁸ g-NK cells, at least at or about 1.0×10⁹ g-NK cells, at least at or about 1.5×10⁹ g-NK cells, at least at or about 2.0×10⁹ g-NK cells, at least at or about 3.0×10⁹ g-NK cells, at least at or about 4.0×10⁹ g-NK cells, at least at or about 5.0×10⁹ g-NK cells or more, at least at or about 1.0×10¹⁰ g-NK cells or more, at least at or about 1.5×10¹⁰ g-NK cells or more, at least at or about 2.0×10¹⁰ g-NK cells or more, or at least at or about 2.5×10¹⁰ g-NK cells or more.

In some embodiments, the provided methods result in the preferential expansion of g-NK cells. In some aspects, g-NK cells are identified by the presence, absence or level of surface expression of one or more various marker that distinguishes NK cells from other lymphocytes or immune cells and that distinguishes g-NK cells from conventional NK cells. In embodiments, surface expression can be determined by flow cytometry, for example, by staining with an antibody that specifically bind to the marker and detecting the binding of the antibody to the marker. Similar methods can be carried out to assess expression of intracellular markers, except that such methods typically include methods for fixation and permeabilization before staining to detect intracellular proteins by flow cytometry. In some embodiments, fixation is achieved using formaldehyde (e.g. 0.01%) followed by disruption of membranes using a detergent (e.g. 0.1% to 1% detergent, for example at or about 0.5%), such as Triton, NP-50, Tween 20, Saponin, Digitonin or Leucoperm.

Antibodies and other binding entities can be used to detect expression levels of marker proteins to identify, detect, enrich and/or isolate the g-NK cells. Suitable antibodies may include polyclonal, monoclonal, fragments (such as Fab fragments), single chain antibodies and other forms of specific binding molecules.

In some embodiments, a cell (e.g. NK cell subset) is positive (pos) for a particular marker if there is detectable presence on or in the cell of a particular marker, which can be an intracellular marker or a surface marker. In embodiments, surface expression is positive if staining is detectable at a level substantially above the staining detected carrying out the same procedures with an isotype-matched control under otherwise identical conditions and/or at a level substantially similar to, or in some cases higher than, a cell known to be positive for the marker and/or at a level higher than that for a cell known to be negative for the marker.

In some embodiments, a cell (e.g. NK cell subset) is negative (neg) for a particular marker if there is an absence of detectable presence on or in the cell of a particular marker, which can be an intracellular marker or a surface marker. In embodiments, surface expression is negative if staining is not detectable at a level substantially above the staining detected carrying out the same procedures with an isotype-matched control under otherwise identical conditions and/or at a level substantially lower than a cell known to be positive for the marker and/or at a level substantially similar to a cell known to be negative for the marker.

In some embodiments, a cell (e.g. NK cell subset) is low (lo or min) for a particular marker if there is a lower level of detectable presence on or in the cell of a particular marker compared to a cell known to be positive for the marker. In embodiments, surface expression can be determined by flow cytometry, for example, by staining with an antibody that specifically bind to the marker and detecting the binding of the antibody to the marker, wherein expression, either surface or intracellular depending on the method used, is low if staining is at a level lower than a cell known to be positive for the marker.

In some embodiments, g-NK cells are cells having a phenotype of NK cells (e.g. CD45^{pos}, CD3^{neg} and/or CD56^{pos}) and express one or more markers that identify or that are associated with a g-NK cell subset.

In some embodiments, g-NK cells are identified as described in published Patent Appl. No. US2013/0295044 or Zhang et al. (2013) J. Immunol., 190:1402-1406.

In some embodiments, g-NK cells are cells that do not express substantial FcRγ but do express at least one marker for natural killer cells. An amino acid sequence for FcRγ chain (Homo sapiens, also called the High affinity immunoglobulin gamma Fc receptor I) is available in the NCBI database as accession number NP_004097.1 (GI:4758344), and is reproduced below as SEQ ID NO:34.

(SEQ ID NO: 34)
MIPAVVLLLLLLVEQAAALGEPQLCYILDAILFLYGIVLTLLYCRLKIQ
VRKAAITSYEKSDGVYTGLSTRNQETYETLKHEKPPQ

In some embodiments, the g-NK cell subset of NK cells can be detected by observing whether FcRγ is expressed by a population of NK cells or a subpopulation of NK cells. In some cases, g-NK cells are identified as cells that do not express FcRγ. FcRγ protein is an intracellular protein. Thus, in some aspects, the presence or absence of FcRγ can be detected after treatment of cells, for example, by fixation and permeabilization, to allow intracellular proteins to be detected. In some embodiments, cells are further assessed for one or more surface markers (CD45, CD3 and/or CD56) prior to the intracellular detection, such as prior to fixation of cells. In some embodiments, g-NK cells are identified, detected, enriched and/or isolated as cells that are CD45^{pos}/CD3^{neg}/CD56^{pos}/FcRγ^{neg}.

In some embodiments, greater than at or about 50% of NK cells in the expanded population are FcRγ^{neg}. In some embodiments, greater than at or about 60% of NK cells in the expanded population are FcRγ^{neg}. In some embodiments, greater than at or about 70% of NK cells in the expanded population are FcRγ^{neg}. In some embodiments, greater than at or about 80% of NK cells in the expanded population are FcRγ^{neg}. In some embodiments, greater than at or about 90% of NK cells in the expanded population are FcRγ^{neg}. In some embodiments, greater than at or about 95% of NK cells in the expanded population are FcRγ^{neg}. For example, the methods herein generally result in a highly pure, e.g. 70-90%, g-NK cell product.

In some embodiments, it may be useful to detect expression of g-NK cells without employing intracellular staining, such as, for example, if cells of the sample are to be subjected to cell sorting or a functional assay. While treatments, e.g. fixation and permeabilization, to permit intracellular staining of FcRγ can be used to confirm the identity of a substantially pure population of cells, in many cases cell-surface markers can be employed that can be detected without injuring the cells when identifying, detecting or isolating g-NK cells. Thus, in some embodiments, g-NK cells are identified using a surrogate marker profile that correlates with the lack of FcRγ among a subset of NK cells. In some embodiments, a surrogate marker profile is of particular use when the presence or absence of an intracellular protein, such as FcRγ, is difficult or not possible to assess depending on the particular application of the cells.

It is found herein that certain combinations of cell surface marker correlate with the g-NK cell phenotype, i.e. cells that lack or are deficient in intracellular expression of FcRγ, thereby providing a surrogate marker profile to identify or detect g-NK cells in a manner that does not injure the cells. In some embodiments, a surrogate marker profile for g-NK cells provided herein is based on positive surface expression of one or more markers CD16 (CD16^{pos}), NKG2C (NKG2C^{pos}), or CD57 (CD57pos) and/or based on low or negative surface expression of one or more markers CD7 (CD7^{dim/neg}), CD161 (CD161^{neg}) and/or NKG2A (NKG2A^{neg}). In some embodiments, cells are further assessed for one or more surface markers of NK cells, such as CD45, CD3 and/or CD56. In some embodiments, g-NK cells can be identified, detected, enriched and/or isolated with the surrogate marker profile CD45^{pos}/CD3^{neg}/CD56^{pos}/CD16^{pos}/CD57^{pos}/CD7^{dim/neg}/CD161^{neg}. In some embodiments, g-NK cells are identified, detected, enriched and/or isolated with the surrogate marker profile CD45^{pos}/CD3^{neg}/CD56^{pos}/NKG2A^{neg}/CD161^{neg}. In some embodiments, g-NK cells that are NKG2C^{pos} and/or NKG2A^{neg} are identified, detected, enriched for, and/or isolated.

In some embodiments, greater than at or about 30% of NK cells in the expanded population are positive for NKG2C and/or greater than at or about 50% of NK cells in the expanded population are negative or low for NKG2A. In some embodiments, greater than at or about 35% of NK cells in the expanded population are positive for NKG2C and/or greater than at or about 60% of NK cells in the expanded population are negative or low for NKG2A. In some embodiments, greater than at or about 40% of NK cells in the expanded population are positive for NKG2C and/or greater than at or about 70% of NK cells in the expanded population are negative or low for NKG2A. In some embodiments, greater than at or about 45% of NK cells in the expanded population are positive for NKG2C and/or greater than at or about 80% of NK cells in the expanded population are negative or low for NKG2A. In some embodiments, greater than at or about 50% of NK cells in the expanded population are positive for NKG2C and/or greater than at or about 85% of NK cells in the expanded population are negative or low for NKG2A. In some embodiments, greater than at or about 55% of NK cells in the expanded population are positive for NKG2C and/or greater than at or about 90% of NK cells in the expanded population are negative or low for NKG2A. In some embodiments, greater than at or about 60% of NK cells in the expanded population are positive for NKG2C and/or greater than at or about 95% of NK cells in the expanded population are negative or low for NKG2A.

In some embodiments, greater than at or about 70% of the g-NK cells in the expanded population are positive for perforin, and greater than at or about 70% of the g-NK cells in the expanded population are positive for granzyme B. In some embodiments, greater than at or about 75% of the g-NK cells in the expanded population are positive for perforin, and greater than at or about 75% of the g-NK cells in the expanded population are positive for granzyme B. In some embodiments, greater than at or about 80% of the g-NK cells in the expanded population are positive for perforin, and greater than at or about 80% of the g-NK cells in the expanded population are positive for granzyme B. In some embodiments, greater than at or about 85% of the g-NK cells in the expanded population are positive for perforin, and greater than at or about 85% of the g-NK cells in the expanded population are positive for granzyme B. In some embodiments, greater than at or about 90% of the g-NK cells in the expanded population are positive for perforin, and greater than at or about 90% of the g-NK cells in the expanded population are positive for granzyme B. In some embodiments, greater than at or about 95% of the g-NK cells in the expanded population are positive for perforin, and greater than at or about 95% of the g-NK cells in the expanded population are positive for granzyme B.

In some of any such embodiments, greater than at or about 20%, greater than at or about 30%, greater than at or about 40%, greater than at or about 50%, greater than at or about 60%, greater than at or about 70%, greater than at or about 80%, greater than at or about 90% or greater than at or about 95% of the total cells in the expanded population comprise a heterologous nucleic acid(s) encoding a CAR. In some of any such embodiments, greater than at or about 20%, greater than at or about 30%, greater than at or about 40%, greater than at or about 50%, greater than at or about 60%, greater than at or about 70%, greater than at or about 80%, greater than at or about 90% or greater than at or about 95% of the total cells in the expanded population comprise a heterologous nucleic acid(s) encoding an immunomodulator (e.g. cytokine, either secretable or membrane-bound as described). In some of any such embodiments, greater than at or about 20%, greater than at or about 30%, greater than at or about 40%, greater than at or about 50%, greater than at or about 60%, greater than at or about 70%, greater than at or about 80%, greater than at or about 90% or greater than at or about 95% of the total cells in the expanded population comprise heterologous nucleic acids encoding a CAR and an immunomodulator (e.g. cytokine, either secretable or membrane-bound as described). In some of any such embodiments, greater than at or about 20%, greater than at or about 30%, greater than at or about 40%, greater than at or about 50%, greater than at or about 60%, greater than at or about 70%, greater than at or about 80%, greater than at or about 90% or greater than at or about 95% of the g-NK cells, or of a cell subset with a surrogate marker profile of g-NK cells as described herein, in the expanded population comprise a heterologous nucleic acid(s) encoding a CAR. In some of any such embodiments, greater than at or about 20%, greater than at or about 30%, greater than at or about 40%, greater than at or about 50%, greater than at or about 60%, greater than at or about 70%, greater than at or about 80%, greater than at or about 90% or greater than at or about 95% of the g-NK cells, or of a cell subset with a surrogate marker profile of g-NK cells as described herein, in the expanded population comprise a heterologous nucleic acid(s) encoding an immunomodulator (e.g. cytokine, either secretable or membrane-bound as described). In some of any such embodiments, greater than at or about 20%, greater than at or about 30%, greater than at or about 40%, greater than at or about 50%, greater than at or about 60%, greater than at or about 70%, greater than at or about 80%, greater than at or about 90% or greater than at or about 95% of the g-NK cells, or of a cell subset with a surrogate marker profile of g-NK cells as described herein, in the expanded population comprise heterologous nucleic acids encoding a CAR and an immunomodulator (e.g. cytokine, either secretable or membrane-bound as described).

Cells expanded by the provided methods can be assessed for any of a number of functional or phenotypic activities, including but not limited to cytotoxic activity, degranulation, ability to produce or secrete cytokines, and expression of one or more intracellular or surface phenotypic markers. Methods to assess such activities are known and are exemplified herein and in working examples.

In some embodiments, antibody-dependent cell cytotoxicity (ADCC) cytotoxic activity against target cells can be used as a measure of functionality. For the ADCC cytotoxicity assays, cells from expansions can be co-cultured with appropriate targets cells in the presence or absence of an antibody specific to a target antigen on the target cells. For example, for anti-myeloma cytotoxicity any of a number of multiple myeloma (MM) target cells can be used (e.g. AM01, KMS11, KMS18, KMS34, LP1 or MM.1S) can be used and the assay performed with an anti-CD38 (e.g. Daratumumab) or anti-CD319 antibody (e.g. Elotuzumab). Cell killing can be determined by any number of methods. For example, cells can be stained with Propidium iodide (PI) and the number of NK-cells, live target cells, and dead target cells can be resolved, such as by flow cytometry.

In some embodiments, greater than at or about 10% of g-NK cells in the expanded population are capable of degranulation against tumor cells. Degranulation can be measured by assessing expression of CD107A. For example, in some embodiments, greater than at or about 20% of g-NK cells in the expanded population are capable of degranulation against tumor cells. In some embodiments, greater than at or about 30% of g-NK cells in the expanded population are capable of degranulation against tumor cells. In some embodiments, greater than at or about 40% of g-NK cells in the expanded population are capable of degranulation against tumor cells. In some embodiments, capacity for degranulation is measured in the absence of an antibody against the tumor cells.

In some embodiments, greater than at or about 10% of g-NK cells in the expanded population are capable of producing an effector cytokine, such as interferon-gamma or TNF-alpha, against tumor cells. In some embodiments, greater than at or about 20% of g-NK cells in the expanded population are capable of producing an effector cytokine, e.g. interferon-gamma or TNF-alpha, against tumor cells. In some embodiments, greater than at or about 30% of g-NK cells in the expanded population are capable of producing an effector cytokine, e.g. interferon-gamma or TNF-alpha, against tumor cells. In some embodiments, greater than at or about 40% of g-NK cells in the expanded population are capable of producing an effector cytokine, e.g. interferon-gamma or TNF-alpha, against tumor cells. In some embodiments, capacity for producing interferon-gamma or TNF-alpha is measured in the absence of an antibody against the tumor cells.

Provided herein are methods for identifying or detecting g-NK cells in a sample containing a population of cells by employing a surrogate marker profile of g-NK cells. In some embodiments, the methods include contacting a sample of cells with a binding molecule, such as an antibody or antigen-binding fragment that is specific for one or more markers CD16, CD57, CD7, CD161, NKG2C, and/or NKG2A. In some embodiments, the methods further include contacting the sample of cells with a binding molecule, such as an antibody or antigen-binding fragment that is specific for CD45, CD3 and/or CD56. In some embodiments of the methods, the one or more binding molecules can be contacted with the sample simultaneously. In some embodiments of the methods, the one or more binding molecules can be contacted with the sample sequentially. In some embodiments, following the contact, the methods can include one or more washing under conditions to retain cells that have bound to the one or more binding molecule and/or to separate away unbound binding molecules from the sample.

In some embodiments, each of the one or more binding molecules, e.g. antibody, may be attached directly or indirectly to a label for detection of cells positive or negative for the marker. For example, the binding molecule, e.g. antibody, may be conjugated, coupled or linked to the label. Labels are well known by one of skill in the art. Labels contemplated herein include, but are not limited to, fluorescent dyes, fluorescent proteins, radioisotopes, chromophores, metal ions, gold particles (e.g., colloidal gold particles), silver particles, particles with strong light scattering properties, magnetic particles (e.g., magnetic bead particles such as Dynabeads® magnetic beads), polypeptides (e.g., FLAG™ tag, human influenza hemagglutinin (HA) tag, etc.), enzymes such as peroxidase (e.g., horseradish peroxidase) or a phosphatase (e.g., alkaline phosphatase), streptavidin, biotin, luminescent compounds (e.g., chemiluminescent substrates), oligonucleotides, members of a specific binding pair (e.g., a ligands and its receptor) and other labels well known in the art that are used for visualizing or detecting a binding molecule, e.g. an antibody, when directly or indirectly attached to said antibody.

A number of well-known methods for assessing expression level of surface markers or proteins may be used, such as detection by affinity-based methods, e.g., immunoaffinity-based methods, e.g., in the context of surface markers, such as by flow cytometry. In some embodiments, the label is a fluorophore and the methods for detection or identification of g-NK cells is by flow cytometry. In some embodiments, different labels are used for each of the different markers by multicolor flow cytometry.

In some embodiments, the methods include contacting a sample with a binding molecule specific to CD45, CD3, CD56, CD57, CD7 and CD161. In some such embodiments, g-NK cells are identified or detected as cells having the g-NK cell surrogate marker profile CD45^{pos}/CD3^{neg}/CD56^{pos}/CD16^{pos}/CD57^{pos}/CD7^{dim/neg}/CD161^{neg}.

In some embodiments, the methods include contacting a sample with a binding molecule specific to CD45, CD3, CD56, NKG2A and CD161. In some such embodiments, g-NK cells are identified or detected as cells having the g-NK cell surrogate marker profile CD45^{pos}/CD3^{neg}/CD56^{pos}/NKG2A^{neg}/CD161^{neg}.

In some embodiments, the provided methods also can include isolating or enriching g-NK, such as g-NK cells preferentially expanded in accord with any of the provided methods. In some such embodiments, a substantially pure population of g-NK cells can be obtained, such as a cell population containing greater than or greater than about 90%, 91%, 92%, 93%, 94%, 95%, 96%, 97%, 98%, 99% or more g-NK cells, such as determined using any of the described panel or combinations of markers. Antibodies and other binding molecules can be used to detect the presence or absence of expression levels of marker proteins, for use in isolating or enriching g-NK cells. In some embodiments, isolation or enrichment is carried out by fluorescence activated cell sorting (FACs). In examples of such methods, g-NK cells are identified or detected by flow cytometry using the methods as described above for staining cells for multiple cell surface markers and stained cells are carried in a fluidic stream for collection of cells that are positive or negative for markers associated with g-NK cells.

### VI. KITS AND ARTICLES OF MANUFACTURE

Provided herein are articles of manufacture and kits comprising the provided compositions containing NK cells enriched for particular subsets, such as g-NK cells. In some embodiments, the g-NK cells are engineered, such as with a nucleic acid encoding an antigen receptor, such as a CAR. In some embodiments, the g-NK cells are engineered, such as with a nucleic acid encoding an immunomodulator, such as a cytokine. In some embodiments, the g-NK cells are engineered, such as with nucleic acids encoding an antigen receptor, such as a CAR and an immunomodulator, such as a cytokine. Among cells in the compositions are engineered g-NK cells that comprise a heterologous nucleic acid encoding an antigen receptor (e.g. CAR). Among cells in the compositions are engineered g-NK cells that comprise a heterologous nucleic acid encoding an immunomodulator (e.g. cytokine). Among cells in the compositions are engineered g-NK cells that comprise heterologous nucleic acids encoding an antigen receptor (e.g. CAR) and an immunomodulator (e.g. cytokine). In some embodiments, the compositions are produced by any of the provided methods. In some embodiments, the kit comprises any of the provided compositions and instructions for administering the composition as a monotherapy.

In some embodiments, provided herein is a kit comprising any of the provided compositions and an additional agent. Exemplary additional agents include any as described herein. In some embodiments, the additional agent comprises an Fc domain. In some embodiment the additional agent is an Fc fusion protein or an antibody. In some embodiments, the additional agent is an antibody. In some embodiments, the additional agent is a human, humanized, or chimeric antibody. In some of these embodiments, the additional agent is a full length antibody. Exemplary antibodies included any as described.

Kits can optionally include one or more components such as instructions for use, devices and additional reagents (e.g., sterilized water or saline solutions for dilution of the compositions and/or reconstitution of lyophilized protein), and components, such as tubes, containers and syringes for practice of the methods. In some embodiments, the kits can further contain reagents for collection of samples, preparation and processing of samples, and/or reagents for quantitating the amount of one or more surface markers in a sample, such as, but not limited to, detection reagents, such as antibodies, buffers, substrates for enzymatic staining, chromagens or other materials, such as slides, containers, microtiter plates, and optionally, instructions for performing the methods. Those of skill in the art will recognize many other possible containers and plates and reagents that can be used in accord with the provided methods.

In some embodiments, the kits can be provided as articles of manufacture that include packing materials for the packaging of the cells, antibodies or reagents, or compositions thereof, or one or more other components. For example, the kits can contain containers, bottles, tubes, vial and any packaging material suitable for separating or organizing the components of the kit. The one or more containers may be formed from a variety of materials such as glass or plastic. In some embodiments, the one or more containers hold a composition comprising cells or an antibody or other reagents for use in the methods. The article of manufacture or kit herein may comprise the cells, antibodies or reagents in separate containers or in the same container.

In some embodiments, the one or more containers holding the composition may be a single-use vial or a multi-use vial, which, in some cases, may allow for repeat use of the composition. In some embodiments, the article of manufacture or kit may further comprise a second container comprising a suitable diluent. The article of manufacture or kit may further include other materials desirable from a commercial, therapeutic, and user standpoint, including other buffers, diluents, filters, needles, syringes, therapeutic agents and/or package inserts with instructions for use.

In some embodiments, the kit can, optionally, include instructions. Instructions typically include a tangible expression describing the cell composition, reagents and/or antibodies and, optionally, other components included in the kit, and methods for using such. In some embodiments, the instructions indicate methods for using the cell compositions and antibodies for administration to a subject for treating a disease or condition, such as in accord with any of the provided embodiments. In some embodiments, the instructions are provided as a label or a package insert, which is on or associated with the container. In some embodiments, the instructions may indicate directions for reconstitution and/or use of the composition.

### VII. METHODS OF TREATMENT

Provided herein are compositions for use and methods relating to the provided g-NK cell compositions comprising engineered g-NK cells (also referred to as engineered g-NK cell compositions), including any as described herein, for use in treating diseases or conditions in a subject. The methods and uses may include use of any composition as described herein, including those described in Section IV or produced by methods herein as described in Section II or V. In some embodiments, provided herein is a method of treating a condition in an individual, comprising administering any of the provided g-NK compositions comprising engineered g-NK cells to an individual in need thereof. Among cells in the compositions are engineered g-NK cells that comprise a heterologous nucleic acid encoding an antigen receptor (e.g. CAR). Among cells in the compositions are engineered g-NK cells that comprise a heterologous nucleic acid encoding an immunomodulator (e.g. cytokine). Among cells in the compositions are engineered g-NK cells that comprise heterologous nucleic acids encoding an antigen receptor (e.g. CAR) and an immunomodulator (e.g. cytokine). In particular embodiments, the composition is produced by the methods provided herein. Such methods and uses include therapeutic methods and uses, for example, involving administration of the therapeutic cells, or compositions containing the same, to a subject having a disease, condition, or disorder. In some cases, the disease or disorder is a tumor or cancer. In some embodiments, the disease or disorder is a virus infection. In some embodiments, the cells or pharmaceutical composition thereof is administered in an effective amount to effect treatment of the disease or disorder. Uses include uses of the cells or pharmaceutical compositions thereof in such methods and treatments, and in the preparation of a medicament in order to carry out such therapeutic methods. In some embodiments, the methods thereby treat the disease or condition or disorder in the subject.

In some embodiments, any of the provided methods and uses may be of provided NK cell compositions comprising engineered g-NK cells may include methods and uses as described in PCT Publication No. WO2020/107002 or PCT Appl. No. PCT/US2021/028504.

In some embodiments, the methods of treatment or uses involve administration of an effective amount of cells of a g-NK cell composition provided herein, such as compositions containing engineered g-NK cells as provided herein, including any such composition that includes expanded NK cells produced by the provided methods, to an individual. In some embodiments, from at or about 10⁵ to at about 10¹², or from at or about 10⁵ and at or about 10⁸, or from at or about 10⁶ and at or about 10¹², or from at or about 10⁸ and at or about 10¹¹, or from at or about 10⁹ and at or about 10¹⁰ of such a g-NK cell composition provided herein, such as a composition containing engineered NK cells as provided herein, including any composition produced by the provided methods, is administered to an individual subject. In some embodiments, a dose of cells containing at or greater than at or about 10⁵, at or greater than at or about 10⁶, at or greater than at or about 10⁷, at or greater than at or about 10⁸, at or greater than at or about 10⁹, at or greater than at or about 10¹⁰, at or greater than at or about 10¹¹, or at or greater than at or about 10¹² of cells of such a g-NK cell composition provided herein, such as a composition containing engineered NK cells as provided herein, including any composition produced by the provided methods, are administered to the individual.

In some embodiments, the methods of treatment or uses involve administration of an effective amount of cells of any of the provided NK cell compositions, including any engineered g-NK cell composition as described herein, to an individual. In some embodiments, from at or about 10⁵ to at about 10¹², or from at or about 10⁵ and at or about 10⁸, or from at or about 10⁶ and at or about 10¹², or from at or about 10⁸ and at or about 10¹¹, or from at or about 10⁹ and at or about 10¹⁰ of cells from any of the provided compositions containing engineered g-NK cells is administered to an individual subject. In some embodiments, a dose of cells containing at or greater than at or about 10⁵, at or greater than at or about 10⁶, at or greater than at or about 10⁷, at or greater than at or about 10⁸, at or greater than at or about 10⁹, at or greater than at or about 10¹⁰, at or greater than at or about 10¹¹, or at or greater than at or about 10¹² of cells from any of the provided compositions containing engineered g-NK cells are administered to the individual. In some embodiments, from or from about 10⁶ to 10¹⁰ of such cells of any of the provided compositions containing engineered g-NK cells per kg are administered to the subject.

In some embodiments, the composition containing engineered g-NK cells may be administered once weekly for a predetermined number of doses.

In some embodiments, the predetermined number of once weekly doses is one dose, two doses, three doses, four doses, five doses, six doses, seven doses, eight doses, nine doses, ten doses, eleven doses or twelve doses. In some embodiments, the once weekly doses are administered for 4 weeks, 6 weeks, 8 weeks, 10 weeks, 12 weeks, 16 weeks, 20 weeks, 24 weeks, 28 weeks, 32 weeks, 36 weeks or more. In some embodiments, six (6) once weekly doses of the g-NK cell composition is administered. In some embodiments, the once weekly doses are administered in consecutive weeks.

In some embodiments the once weekly dose is administered in a cycling regimen. In some embodiments, the cycling regimen is a 14 day cycle. In some embodiments, the once weekly dose is administered two times in the 14 day cycle. In some embodiments, the 14 day cycle is repeated twice. In some embodiments, the 14 day cycle is repeated three times.

In some embodiments, an effective amount of any of the disclosed cells or compositions containing engineered g-NK cells disclosed herein is administered to a subject once weekly, for a duration of five weeks.

In some embodiments, each dose of cells of a g-NK cell composition containing engineered g-NK cells may be from at or about from at or about 1×10⁸ cells to at or about 50×10⁹ cells of the g-NK cell composition. In some embodiments, each dose of cells of a g-NK cell composition containing engineered g-NK cells may be or may be about 5×10⁸ cells of the g-NK cell composition. In some embodiments, each dose of cells of a g-NK cell composition containing engineered g-NK cells may be or may be about 5×10⁹ cells of the g-NK cell composition. In some embodiments, each dose of cells of a g-NK cell composition containing engineered g-NK cells may be or may be about 10×10⁹ cells of the g-NK cell composition.

In some embodiments, the dose for administration in accord with any of the provided methods of treatment or uses is from at or about 1×10⁵ cells/kg to at or about 1×10⁷ cells/kg, such as from at or about 1×10⁵ cells/kg to at or about 7.5×10⁶ cells/kg, from at or about 1×10⁵ cells/kg to at or about 5×10⁶ cells/kg, from at or about 1×10⁵ cells/kg to at or about 2.5×10⁶ cells/kg, from at or about 1×10⁵ cells/kg to at or about 1×10⁶ cells/kg, from at or about 1×10⁵ cells/kg to at or about 7.5×10⁵ cells/kg, from at or about 1×10⁵ cells/kg to at or about 5×10⁵ cells/kg, from at or about 1×10⁵ cells/kg to at or about 2.5×10⁵ cells/kg, from at or about 2.5×10⁵ cells/kg to at or about 1×10⁷ cells/kg, from at or about 2.5×10⁵ cells/kg to at or about 7.5×10⁶ cells/kg, from at or about 2.5×10⁵ cells/kg to at or about 5×10⁶ cells/kg, from at or about 2.5×10⁵ cells/kg to at or about 2.5×10⁶ cells/kg, from at or about 2.5×10⁵ cells/kg to at or about 1×10⁶ cells/kg, from at or about 2.5×10⁵ cells/kg to at or about 7.5×10⁵ cells/kg, from at or about 2.5×10⁵ cells/kg to at or about 5×10⁵ cells/kg, from at or about 5×10⁵ cells/kg to at or about 1×10⁷ cells/kg, from at or about 5×10⁵ cells/kg to at or about 7.5×10⁶ cells/kg, from at or about 5×10⁵ cells/kg to at or about 5×10⁶ cells/kg, from at or about 5×10⁵ cells/kg to at or about 2.5×10⁶ cells/kg, from at or about 5×10⁵ cells/kg to at or about 1×10⁶ cells/kg, from at or about 5×10⁵ cells/kg to at or about 7.5×10⁵ cells/kg, from at or about 1×10⁶ cells/kg to at or about 1×10⁷ cells/kg, from at or about 1×10⁶ cells/kg to at or about 7.5×10⁶ cells/kg, from at or about 1×10⁶ cells/kg to at or about 5×10⁶ cells/kg, from at or about 1×10⁶ cells/kg to at or about 2.5×10⁶ cells/kg, from at or about 2.5×10⁶ cells/kg to at or about 1×10⁷ cells/kg, from at or about 2.5×10⁶ cells/kg to at or about 7.5×10⁶ cells/kg, from at or about 2.5×10⁶ cells/kg to at or about 5×10⁶ cells/kg, from at or about 5×10⁶ cells/kg to at or about 1×10⁷ cells/kg, from at or about 5×10⁶ cells/kg to at or about 7.5×10⁶ cells/kg, or from at or about 7.5×10⁶ cells/kg to at or about 1×10⁷ cells/kg. In some embodiments, the dose for administration is from at or about 1×10⁵ cells/kg to at or about 1×10⁸ cells/kg, such as from at or about 2.5×10⁵ cells/kg to at or about 1×10⁸ cells/kg, from at or about 5×10⁵ cells/kg to at or about 1×10⁸ cells/kg, from at or about 7.5×10⁵ cells/kg to at or about 1×10⁸ cells/kg, from at or about 1×10⁶ cells/kg to at or about 1×10⁸ cells/kg, from at or about 2.5×10⁶ cells/kg to at or about 1×10⁸ cells/kg, from at or about 5×10⁶ cells/kg to at or about 1×10⁸ cells/kg, from at or about 7.5×10⁶ cells/kg to at or about 1×10⁸ cells/kg, from at or about 1×10⁷ cells/kg to at or about 1×10⁸ cells/kg, from at or about 2.5×10⁷ cells/kg to at or about 1×10⁸ cells/kg, from at or about 5×10⁷ cells/kg to at or about 1×10⁸ cells/kg, or from at or about 7.5×10⁷ cells/kg to at or about 1×10⁸ cells/kg.

In some embodiments, the dose is given as the number of g-NK cells or an NK cell subset in the composition that is associated with or includes a surrogate marker for g-NK cells, such as any of the NK cell subsets described herein, or a number of viable cells of any of the foregoing. In any of the above embodiments, the dose is given as the number of cells in a composition of engineered cells as provided, such as produced by the provided methods, or a number of viable cells of any of the foregoing.

In some embodiments, the dose for administration in accord with any of the methods of treatment or uses is from at or about 5×10⁷ to at or about 10×10⁹, such as from at or about 5×10⁷ to at or about 5×10⁹, from about or about 5×10⁷ to at or about 1×10⁹, from at or about 5×10⁷ to at or about 5×10⁸, from about or about 5×10⁷ to at or about 1×10⁸, 1×10⁸ to at or about 10×10⁹, from at or about 1×10⁸ to at or about 5×10⁹, from about or about 1×10⁸ to at or about 1×10⁹, from at or about 1×10⁸ to at or about 5×10⁸, from at or about 5×10⁸ to at or about 10×10⁹, from at or about 5×10⁸ to at or about 5×10⁹, from about or about 5×10⁸ to at or about 1×10⁹, from at or about 1×10⁹ to at or about 10×10⁹, from at or about 1×10⁹ to at or about 5×10⁹, or from at or about 5×10⁹ to at or about 10×10⁹ of the g-NK cell composition containing engineered g-NK cells. In some embodiments, the dose for administration is at or about 5×10⁸ cells of the g-NK cell composition containing engineered g-NK cells. In some embodiments, the dose for administration is at or about 1×10⁹ cells of the g-NK cell composition containing engineered g-NK cells. In some embodiments, the dose for administration is at or about 5×10⁹ cells of the g-NK cell composition containing engineered g-NK cells. In some embodiments, the dose for administration is at or about 1×10¹⁰ cells of the g-NK cell composition containing engineered g-NK cells. In any of the above embodiments, the dose is given as the number of cells in a composition of engineered cells produced by the provided method, or a number of viable cells of any of the foregoing. In some embodiments, the dose is given as the number of g-NK cells or an NK cell subset that is associated with or includes a surrogate marker for g-NK cells, such as any of the NK cell subsets described herein, or a number of viable cells of any of the foregoing.

In some embodiments, a dose of cells of a composition containing engineered g-NK cells are administered to an individual soon after expansion and/or engineering according to the provided methods. In other embodiments, the composition of g-NK cells containing engineered g-NK cells are stored prior to administration, such as by methods described above. For example, the NK cells can be stored for greater than 6, 12, 18, or 24 months prior to administration to the individual.

In some embodiments, the provided compositions containing engineered NK cells and subsets thereof, such as g-NK cells, can be administered to a subject by any convenient route including parenteral routes such as subcutaneous, intramuscular, intravenous, and/or epidural routes of administration.

In particular embodiments, the provided compositions are administered by intravenous infusion. In some embodiments, at or about 10×10⁶ cells to 10×10⁹ cells are administered by intravenous infusion in a volume of 1 mL to 100 mL. In some embodiments, at or about 50×10⁶ cells are administered. In some embodiments, at or about 1×10⁹ cells are administered. In some embodiments, at or about 5×10⁹ cells are administered. In some embodiments, at or about 10×10⁹ cells are administered. It is within the level of a skilled artisan to determine the volume of cells for infusion to administer the number of cells. In one example, 0.5×10⁹ cells is administered by intravenous infusion of a volume of about 20 mL from a composition, such as a thawed cryopreserved composition, formulated at a concentration of at or about 2.5×10⁷ cells/mL (e.g. at or about 5×10⁹ cells in 200 mL).

The provided engineered g-NK cell compositions can be used in methods of treating an individual with a tumor or hyperproliferative disorders or microbial infection such as a viral infection, yeast infection, fungal infection, protozoan infection and/or bacterial infection. The provided engineered g-NK cell compositions can be administered for treatment of animals, such as mammalian animals, for example human subjects.

In some examples, the methods include treating a hyperproliferative disorder, such as a hematological malignancy or a solid tumor. Examples of types of cancer and proliferative disorders that can be treated with the compositions described herein include, but are not limited to, multiple myeloma, leukemia (e.g., myeloblastic, promyelocytic, myelomonocytic, monocytic, erythroleukemia, chronic myelocytic (granulocytic) leukemia, and chronic lymphocytic leukemia), lymphoma (e.g., Hodgkin's disease and non-Hodgkin's disease), fibrosarcoma, myxosarcoma, liposarcoma, chondrosarcoma, osteogenic sarcoma, angiosarcoma, endotheliosarcoma, Ewing's tumor, colon carcinoma, pancreatic cancer, breast cancer, ovarian cancer, prostate cancer, squamous cell carcinoma, basal cell carcinoma, adenocarcinoma, renal cell carcinoma, hepatoma, Wilm's tumor, cervical cancer, uterine cancer, testicular tumor, lung carcinoma, small cell lung carcinoma, bladder carcinoma, epithelial carcinoma, glioma, astrocytoma, oligodendroglioma, melanoma, neuroblastoma, retinoblastoma, dysplasia and hyperplasia. The treatment and/or prevention of cancer includes, but is not limited to, alleviating one or more symptoms associated with cancer, the inhibition or reduction of the progression of cancer, the promotion of the regression of cancer, and/or the promotion of the immune response.

In some examples, the methods include treating a viral infection, such as an infection caused by the presence of a virus in the body. Viral infections may be caused by DNA or RNA viruses and include chronic or persistent viral infections, which are viral infections that are able to infect a host and reproduce within the cells of a host over a prolonged period of time-usually weeks, months or years, before proving fatal. Viruses giving rise to chronic infections that which may be treated in accordance with the present invention include, for example, the human papilloma viruses (HPV), Herpes simplex, and other herpes viruses, the viruses of hepatitis B and C as well as other hepatitis viruses, human immunodeficiency virus, and the measles virus, all of which can produce important clinical diseases. Prolonged infection may ultimately lead to the induction of disease which may be, e.g., in the case of hepatitis C virus liver cancer, fatal to the patient. Other chronic viral infections which may be treated in accordance with the present invention include Epstein Barr virus (EBV), as well as other viruses such as those which may be associated with tumors.

Examples of viral infections which can be treated or prevented with the compositions and methods described herein include, but are limited to, viral infections caused by coronaviruses (e.g., SARS-CoV-2, wherein the infection is COVID-19), retroviruses (e.g., human T-cell lymphotrophic virus (HTLV) types I and II and human immunodeficiency virus (HIV)), herpes viruses (e.g., herpes simplex virus (HSV) types I and II, Epstein-Ban virus and cytomegalovirus), arenaviruses (e.g., lassa fever virus), paramyxoviruses (e.g., morbillivirus virus, human respiratory syncytial virus, and pneumovirus), adenoviruses, bunyaviruses (e.g., hantavirus), cornaviruses, filoviruses (e.g., Ebola virus), flaviviruses (e.g., hepatitis C virus (HCV), yellow fever virus, and Japanese encephalitis virus), hepadnaviruses (e.g., hepatitis B viruses (HBV)), orthomyoviruses (e.g., Sendai virus and influenza viruses A, B and C), papovaviruses (e.g., papillomaviruses), picornaviruses (e.g., rhinoviruses, enteroviruses and hepatitis A viruses), poxviruses, reoviruses (e.g., rotaviruses), togaviruses (e.g., rubella virus), and rhabdoviruses (e.g., rabies virus). The treatment and/or prevention of a viral infection includes, but is not limited to, alleviating one or more symptoms associated with said infection, the inhibition, reduction or suppression of viral replication, and/or the enhancement of the immune response.

In some embodiments, the engineered g-NK cells and compositions containing engineered g-NK cells are used in a method of treating a yeast or bacterial infection. Examples of yeast or bacterial infections include infections relating to Streptococcus pyogenes, Streptococcus pneumoniae, Neisseria gonorrhoea, Neisseria meningitidis, Corynebacterium diphtheriae, Clostridium botulinum, Clostridium perfringens, Clostridium tetani, Haemophilus influenzae, Klebsiella pneumoniae, Klebsiella ozaenae, Klebsiella rhinoscleromotis, Staphylococcus aureus, Vibrio cholera, Escherichia coli, Pseudomonas aeruginosa, Campylobacter (Vibrio) fetus, Campylobacter jejuni, Aeromonas hydrophila, Bacillus cereus, Edwardsiella tarda, Yersinia enterocolitica, Yersinia pestis, Yersinia pseudotuberculosis, Shigella dysenteriae, Shigella flexneri, Shigella sonnei, Salmonella typhimurium, Treponema pallidum, Treponema pertenue, Treponema carateneum, Borrelia vincentii, Borrelia burgdorferi, Leptospira icterohemorrhagiae, Mycobacterium tuberculosis, Toxoplasma gondii, Pneumocystis carinii, Francisella tularensis, Brucella aborts, Brucella suis, Brucella melitensis, Mycoplasma spp., Rickettsia prowazeki, Rickettsia tsutsugumushi, Chlamydia spp., Helicobacter pylori or combinations thereof.

In some embodiments, the provided engineered g-NK cells and compositions thereof can be used as a monotherapy for the treatment of the disease or disorder.

### A. Combination Therapy

In some embodiments, compositions containing engineered g-NK cells as provided herein can be administered in a combination therapy with one or more other agents for treating a disease or condition in a subject. In such embodiments, the composition containing engineered g-NK cells as provided herein can be administered prior to, concurrently with or subsequent (after) the administration of one or more other agents.

In some embodiments, the disclosed methods of treating a subject with the provided engineered g-NK cells and compositions can be in combination with a therapeutic monoclonal antibody, such as an anti-tumor antigen or anti-cancer antibody, anti-viral antibody or anti-bacterial antibody.

In some embodiments, a dose of cells of the engineered g-NK cells can be administered simultaneously or sequentially with anti-microbial, anti-viral and other therapeutic agents. In some embodiments, the methods are carried out in combination with administering to the subject a chemotherapeutic agent, a cytotoxic agent, or an immunomodulatory agent.

In some embodiments provided herein, the engineered g-NK cells, or compositions containing the same, can be administered to an individual in combination with cytokines and/or growth factors. As cytokines are necessary for NK cell activity, typical methods involve administering exogenous cytokines to a subject in combination with an NK cell therapy as exogenous cytokine support.

Exemplary combination therapies are described in the following subsections.

### 1. Antibody Combination

In some embodiments, compositions containing engineered g-NK cells as provided herein exhibit enhanced activity when activated by or contacted with antibodies or Fc-containing proteins, such as compared to conventional NK cells. For example, the g-NK cells can be activated by antibody-mediated crosslinking of CD16 or by antibody-coated tumor cells. In some embodiments, provided herein is a method of treating a condition in an individual comprising administering engineered g-NK cells or composition thereof and an antibody to a subject.

One of ordinary skill in the art can select an appropriate therapeutic (e.g., anti-cancer) monoclonal antibody to administer to the subject with the provided engineered g-NK cells and compositions described herein, such as depending on the particular disease or condition of the individual. Suitable antibodies may include polyclonal, monoclonal, fragments (such as Fab fragments), single chain antibodies and other forms of specific binding molecules.

In some embodiments, the antibody may further include humanized or human antibodies. Humanized forms of non-human antibodies are chimeric Igs, Ig chains or fragments (such as Fv, Fab, Fab′, F(ab′)2 or other antigen-binding subsequences of an antibody) that contain minimal sequence derived from non-human Ig. In some embodiments, the antibody comprises an Fc domain.

Generally, a humanized antibody has one or more amino acid residues introduced from a non-human source. These non-human amino acid residues are often referred to as “import” residues, which are typically taken from an “import” variable domain. Humanization is accomplished by substituting rodent CDRs or CDR sequences for the corresponding sequences of a human antibody (Jones et al., 1986; Riechmann et al., 1988; Verhoeyen et al., 1988). Such “humanized” antibodies are chimeric antibodies (1989), wherein substantially less than an intact human variable domain has been substituted by the corresponding sequence from a non-human species. In practice, humanized antibodies are typically human antibodies in which some CDR residues and possibly some Fc residues are substituted by residues from analogous sites in rodent antibodies. Humanized antibodies include human antibodies (recipient antibody) in which residues from a complementary determining region (CDR) of the recipient are replaced by residues from a CDR of a non-human species (donor antibody) such as mouse, rat or rabbit, having the desired specificity, affinity and capacity. In some instances, corresponding non-human residues replace Fv framework residues of the human antibody. Humanized antibodies may comprise residues that are found neither in the recipient antibody nor in the imported CDR or framework sequences. In general, the humanized antibody comprises substantially all of at least one, and typically two, variable domains, in which most if not all of the CDR regions correspond to those of a non-human Ig and most if not all of the FR regions are those of a human antibody consensus sequence. The humanized antibody optimally also comprises at least a portion of an antibody constant region (Fc), typically that of a human antibody (Jones et al., 1986; Presta, 1992; Riechmann et al., 1988).

Human antibodies can also be produced using various techniques, including phage display libraries (Hoogenboom et al., 1991; Marks et al., 1991) and the preparation of human mAbs (Boerner et al., 1991; Reisfeld and Sell, 1985). Similarly, introducing human Ig genes into transgenic animals in which the endogenous antibody genes have been partially or completely inactivated can be exploited to synthesize human Abs. Upon challenge, human antibody production is observed, which closely resembles that seen in humans in all respects, including gene rearrangement, assembly, and antibody repertoire (1997a; 1997b; 1997c; 1997d; 1997; 1997; Fishwild et al., 1996; 1997; 1997; 2001; 1996; 1997; 1997; 1997; Lonberg and Huszar, 1995; Lonberg et al., 1994; Marks et al., 1992; 1997; 1997; 1997).

Specifically, the cells of the present invention can be targeted to tumors by administration with an antibody that recognizes a tumor associated antigen. One of ordinary skill in the art will appreciate that the present g-NK cells are suitable for use with a wide variety of antibodies that recognize tumor associated antigens. Non-limiting examples of a tumor associated antigen includes CD19, CD20, CD22, CD30, CD33, CD37, CD38, CD40, CD52, CD56, CD70, CD74, CD140, EpCAM, CEA, gpA33, mesothelin, α-fetoprotein, Mucin, PDGFR-alpha, TAG-72, CAIX, PSMA, folate-binding protein, scatter factor receptor kinase, a ganglioside, cytokerain, frizzled receptor, VEGF, VEGFR, Integrin αVβ3, integrin α5β1, EGFR, EGFL7, ERBB2 (HER2), ERBB3, fibronectin, HGF, HER3, LOXL2, MET, IGF1R, IGLF2, EPHA3, FR-alpha, phosphatidylserine, Syndecan 1, SLAMF7 (CD319), TRAILR1, TRAILR2, RANKL, FAP, vimentin or tenascin. In some cases, the antibody is an anti-CD20 antibody (e.g. rituximab), an anti-HER2 antibody (e.g. cetuximab), an anti-CD52 antibody, an anti-EGFR antibody and an anti-CD38 antibody (e.g. daratumumab), an anti-SLAMF7 antibody (e.g. elotuzumab).

Non-limiting antibodies that can be used in the provided methods in combination therapy with a cell composition including g-NK cells include Trastuzumab (Herceptin®), Ramucirumab (Cyramza®), Atezolizumab (Tecentriq™), Nivolumab (Opdivo®), Durvalumab (Imfinzi™), Avelumab (Bavencio®), Pembrolizumab (Keytruda®), Bevacizumab (Avastin®), Everolimus (Afinitor®), Pertuzumab (Perjeta®), ado-Trastuzumab emtansine (Kadcyla®), Cetuximab (Erbitux®), Denosumab (Xgeva®), Rituximab (Rituxan®), Alemtuzumab (Campath®), Ofatumumab (Arzerra®), Obinutuzumab (Gazyva®), Necitumumab (Portrazza™), Ibritumomab tiuxetan (Zevalin®), Brentuximab vedotin (Adcetris®), Siltuximab (Sylvant®), Bortezomib (Velcade®), Daratumumab (Darzalex™), Elotuzumab (Empliciti™), Dinutuximab (Unituxin™), Olaratumab (Lartruvo™), Ocrelizumab, Isatuximab, Truxima, Blitzima, Ritemvia, Rituzena, Herzuma, Ruxience, ABP 798, Kanjinti, Ogivry, BI 695500, Novex (RTXM83), Tositumomab or Ontruzant, or a biosimilar thereof. Exemplary antibodies include rituximab, trastuzumab, aletuzumab, certuximab, daratumumab, veltuzumab, ofatumumab, ublituximab, ocaratuzumab or elotuzumab.

In some embodiments, the antibody can be an anti-PD-1 or anti-PD-L 1 antibody. Antibodies targeting PD-1 or PD-LI include, but are not limited to, Nivolumab, Pembrolizumab or Atezolizumab.

Antibodies specific for a selected cancer type can be chosen, and include any antibody approved for treatment of cancer. Examples include trastuzumab (Herceptin) for breast cancer, rituximab (Rituxan®) for lymphoma, and cetuximab (Erbitux) for head and neck squamous cell carcinoma. A skilled artisan is familiar with FDA-approved monoclonal antibodies able to bind particular tumor or disease antigens, any of which can be used in accord with the provided methods for treating the tumor or disease.

In some embodiments, the methods are for treating adenocarcinoma of the stomach or gastroesophageal junction and the antibody is Trastuzumab (Herceptin®) or Ramucirumab (Cyramza®).

In some embodiments, the methods are for treating bladder cancer and the antibody is Atezolizumab (Tecentriq™), Nivolumab (Opdivo®), Durvalumab (Imfinzi™), Avelumab (Bavencio®), or Pembrolizumab (Keytruda®).

In some embodiments, the methods are for treating brain cancer and the antibody is Bevacizumab (Avastin®).

In some embodiments, the methods are for treating breast cancer and the antibody is Trastuzumab (Herceptin®).

In some embodiments, the methods are for treating cervical cancer and the antibody is Bevacizumab (Avastin®).

In some embodiments, the methods are for treating colorectal cancer and the antibody is Cetuximab (Erbitux®), Panitumumab (Vectibix®), Bevacizumab (Avastin®) or Ramucirumab (Cyramza®).

In some embodiments, the methods are for treating endocrine/neuroendocrine tumors and the antibody is Avelumab (Bavencio®).

In some embodiments, the methods are for treating head and neck cancer and the antibody is Cetuximab (Erbitux®), Pembrolizumab (Keytruda®), Nivolumab (Opdivo®), Trastuzumab or Ramucirumab.

In some embodiments, the methods are for treating bone cancer and the antibody is Denosumab (Xgeva®).

In some embodiments, the methods are for treating kidney cancer and the antibody is Bevacizumab (Avastin®) or Nivolumab (Opdivo®).

In some embodiments, the methods are for treating leukemia and the antibody is Rituximab (Rituxan®), Alemtuzumab (Campath®), Ofatumumab (Arzerra®), Obinutuzumab (Gazyva®) or Blinatumomab (Blincyto®).

In some embodiments, the methods are for treating lung cancer and the antibody is Bevacizumab (Avastin®), Ramucirumab (Cyramza®), Nivolumab (Opdivo®), Necitumumab (Portrazza™), Pembrolizumab (Keytruda®) or Atezolizumab (Tecentriq™).

In some embodiments, the methods are for treating lymphoma and the antibody is Ibritumomab tiuxetan (Zevalin®), Brentuximab vedotin (Adcetris®), Rituximab (Rituxan®), Siltuximab (Sylvant®), Obinutuzumab (Gazyva®), Nivolumab (Opdivo®) or Pembrolizumab (Keytruda®).

In some embodiments, the methods are for treating multiple myeloma and the antibodies are Bortezomib (Velcade®), Daratumumab (Darzalex™), or Elotuzumab (Empliciti™).

In some embodiments, the methods are for treating neuroblastoma and the antibody is Dinutuximab (Unituxin™).

In some embodiments, the methods are for treating ovarian epithelial/fallopian tube/primary peritoneal cancer and the antibody is Bevacizumab (Avastin®).

In some embodiments, the method is for treating pancreatic cancer and the antibody is Cetuximab (Erbitux®) or Bevacizumab (Avastin®).

In some embodiments, the method is for treating skin cancer and the antibody is Ipilimumab (Yervoy®), Pembrolizumab (Keytruda®), Avelumab (Bavencio®) or Nivolumab (Opdivo®).

In some embodiments, the method is for treating soft tissue sarcoma and the antibody is Olaratumab (Lartruvo™).

In some embodiments, the subject is administered a population of g-NK cells described herein and an effective dose of a bispecific antibody. In some embodiments, the bispecific antibody comprises a first binding domain and a second binding domain, the first binding domain specifically binding to a surface antigen on an immune cell, for instance an NK cell or a macrophage. In some embodiments, the first binding domain specifically binds to an activating receptor, for instance CD16 (CD16a), on an NK cell or a macrophage. In some embodiments, the second binding domain specifically binds to a tumor-associated antigen. The tumor-associated antigen to target can be chosen based on cancer type and includes, but is not limited to, CD19, CD20, CD22, CD30, CD33, CD37, CD38, CD40, CD52, CD56, CD70, CD74, CD140, EpCAM, CEA, gpA33, mesothelin, α-fetoprotein, Mucin, PDGFR-alpha, TAG-72, CAIX, PSMA, folate-binding protein, scatter factor receptor kinase, a ganglioside, cytokerain, frizzled receptor, VEGF, VEGFR, Integrin αVβ3, integrin α5β1, EGFR, EGFL7, ERBB2 (HER2), ERBB3, fibronectin, HGF, HER3, LOXL2, MET, IGF1R, IGLF2, EPHA3, FR-alpha, phosphatidylserine, Syndecan 1, SLAMF7 (CD319), TRAILR1, TRAILR2, RANKL, FAP, vimentin or tenascin. In some embodiments, the first binding domain specifically binds to CD16, and the second binding domain specifically binds to CD30.

The engineered g-NK cells and the additional agent can be administered sequentially or simultaneously. In some embodiments, the additional agent can be administered before administration of the g-NK cells. In some embodiments, the additional agent can be administered after administration of the engineered g-NK cells. For example, the engineered g-NK cells can be administered simultaneously with antibodies specific for a selected cancer type. Alternatively, the engineered g-NK cells can be administered at selected times that are distinct from the times when antibodies specific for a selected cancer type are administered.

In particular examples, the subject is administered an effective dose of an antibody before, after, or substantially simultaneously with the population containing engineered g-NK cells. In some examples, the subject is administered about 0.1 mg/kg to about 100 mg/kg of the antibody (such as about 0.5-10 mg/kg, about 1-20 mg/kg, about 10-50 mg/kg, about 20-100 mg/kg, for example, about 0.5 mg/kg, about 1 mg/kg, about 2 mg/kg, about 3 mg/kg, about 4 mg/kg, about 5 mg/kg, about 8 mg/kg, about 10 mg/kg, about 16 mg/kg, about 20 mg/kg, about 24 mg/kg, about 36 mg/kg, about 48 mg/kg, about 60 mg/kg, about 75 mg/kg, or about 100 mg/kg). An effective amount of the antibody can be selected by a skilled clinician, taking into consideration the particular antibody, the particular disease or conditions (e.g. tumor or other disorder), the general condition of the subject, any additional treatments the subject is receiving or has previously received, and other relevant factors. The subject is also administered a population of containing engineered g-NK cells described herein. Both the antibody and the population of engineered g-NK cells are typically administered parenterally, for example intravenously; however, injection or infusion to a tumor or close to a tumor (local administration) or administration to the peritoneal cavity can also be used. One of skill in the art can determine appropriate routes of administration.

In some embodiments, the subject for treatment of a virus is administered an effective dose of one or more antibodies against the virus as well as a population of g-NK cells described herein. In some embodiments, the one or more antibodies is an antibody that binds to a spike glycoprotein, for instance a spike glycoprotein of SARS-Cov-2. In some embodiments, the subject is administered a population containing engineered g-NK cells described herein as well an effective dose of an Fc-fusion protein, for instance a recombinant ACE2-Fc fusion protein. In some embodiments, the subject is administered a population of g-NK cells described herein and serum containing antibodies against the virus, for instance antibodies against SARS-Cov-2. In some embodiments, the serum is convalescent serum collected from a patient recovering from an infection caused by the same virus. In some embodiments, convalescent serum from multiple patients recovering from infections caused by the same virus are collected, combined, and administered with a population containing engineered g-NK cells described herein to the subject in need thereof.

### 2. Cytokines and Growth Factors

In some embodiments provided herein, the engineered g-NK cells, or compositions containing the same, can be administered to an individual in combination with cytokines and/or growth factors. In some embodiments provided herein, the engineered g-NK cells, or compositions containing the same, can be administered to an individual in combination with a further exogenously administered cytokine and/or growth factor. As cytokines are necessary for NK cell activity, typical methods involve administering exogenous cytokines to a subject in combination with an NK cell therapy as exogenous cytokine support.

According to some embodiments, the at least one growth factor or cytokine comprises a growth factor selected from the group consisting of SCF, FLT3, IL-2, IL-7, IL-15, IL-12, IL-21, and IL-27.

In some embodiments, at least one cytokine is administered to the subject in combination with administration of the engineered g-NK cells or a composition thereof.

Cytokines are a broad class of proteins that play an important role in cell signaling, particularly in the context of the immune system. Cytokines have been shown to play a role in autocrine, paracrine, and endocrine signaling as immunomodulating agents. Cytokines may function as immunoactivators, stimulating an immune-mediated response, or as immunosuppressants, damping down immune-mediated responses. Cytokines include chemokines, interferons, interleukins, lymphokines, and tumor necrosis factors, but generally not hormones or growth factors.

In some embodiments, the cytokine is an interleukin. Interleukins are a group of cytokines that are generally secreted proteins and signal molecules that mediate a broad range of immune responses. For example, Interleukin (IL)-2 plays a role in regulating the activities of white blood cells, while Interleukin (IL)-15 plays a major role in the development of inflammatory and protective immune responses to microbial invaders and parasites through modulating the activities of cells of both the innate and adaptive immune systems. In some embodiments, one or more activities of NK cells, including g-NK cells as provided, are regulated by IL-2, IL-21 and/or IL-15 or another cytokine as described.

In some embodiments, the interleukin includes a cytokine produced by immune cells such as lymphocytes, monocytes or macrophages. In some embodiments, the cytokine is an immune activating cytokine that can be used to induce NK cells, such as to the promotion of NK cell survival, activation and/or proliferation. For instance, certain cytokines, such as IL-15 or IL-21, may prevent or reduce NK cells from undergoing senescence, such as by improving their ability to expand ex vivo or in vivo. In some embodiments, the interleukin or functional portion thereof is a partial or full peptide of one or more of IL-2, IL-4, IL-6, IL-7, IL-9, IL-10, IL-11, IL-12, IL-15, IL-18, or IL-21. In some embodiments, the cytokine is IL-2, IL-7, IL-12, IL-15, IL-18, IL-21, Flt3-L, SCF, or IL-7. In some embodiments, the cytokine is IL-2. In some embodiments, the cytokine is IL-12. In some embodiments the cytokine is IL-15. In some embodiments, the cytokine is IL-21. In some embodiments, the cytokine may be administered with the respective receptor for the cytokine. In some embodiments, the steps of administering a cytokine with the engineered g-NK cells permits cytokine signaling, thereby maintaining or improving cell growth, proliferation, expansion and/or effector function of the NK cells.

In particular embodiments recombinant IL-2 is administered to the subject. In other particular embodiments, recombinant IL-15 is administered to the subject. In other particular embodiments, recombinant IL-21 is administered to the subject.

In some embodiments, the cytokine is IL-15 or a functional portion thereof. IL-15 is a cytokine that regulates NK cell activation and proliferation. In some cases, IL-15 and IL-12 share similar biological activities. For instance, IL-15 and IL-2 bind common receptor subunits, and may compete for the same receptor. In some embodiments, IL-15 induces the activation of JAK kinases, as well as the phosphorylation and activation of transcription activators STAT3, STAT5, and STAT6. In some embodiments, IL-15 promotes or regulates one or more functional activities of NK cells, such as the promotion of NK cell survival, regulation of NK cell and T cell activation and proliferation as well as the support of NK cell development from hematopoietic stem cells. In some embodiments, a functional portion is a portion of IL-15 (e.g. containing a truncated contiguous sequence of amino acids of full-length IL-15) that retains one or more functions of full length or mature IL-15, such as the promotion of NK cell survival, regulation of NK cell and T cell activation and proliferation as well as the support of NK cell development from hematopoietic stem cells. All or a functional portion of IL-15 can be administered to a subject.

As will be appreciated by those of skill in the art, the sequence of a variety of IL-15 molecules are known in the art. In one aspect, the IL-15 is a wild type IL-15. In some aspects, the IL-15 is a mammalian IL-15 (e.g., Homo sapiens interleukin 15 (IL15), transcript variant 3, mRNA, NCBI Reference Sequence: NM_000585.4; Canis lupus familiaris interleukin 15 (IL15), mRNA, NCBI Reference Sequence: NM_001197188.1; Felis catus interleukin 15 (IL15), mRNA, NCBI Reference Sequence: NM_001009207.1). Examples of “mammalian” or “mammals” include primates (e.g., human), canines, felines, rodents, porcine, ruminants, and the like. Specific examples include humans, dogs, cats, horses, cows, sheep, goats, rabbits, guinea pigs, rats and mice. In a particular aspect, the mammalian IL-15 is a human IL-15. Human IL-15 amino acid sequences include, for example, Genbank Accession Nos: NR_751915.1, NP_000576.1, AA100963.1, AA100964.1, AA100962.1, CAA71044.1, AAH18149.1, AAB97518.1, CAA63914.1, and CAA63913.1.

In some embodiments, the IL-15 nucleotide sequence is set forth in SEQ ID NO:9 or is a sequence that has at least or at least about 85%, at least or at least about 90%, at least or at least about 95%, or at least or at least about 98% sequence identity to SEQ ID NO:9. In some embodiments, the IL-15 is in a mature form lacking the signal peptide sequence and in some cases also the propeptide sequence. In some embodiments, the IL-15 has the sequence of amino acids set forth in SEQ ID NO:2 or a sequence that has at least or at least about 85%, at least or at least about 90%, at least or at least about 95%, or at least or at least about 98% sequence identity to SEQ ID NO:2.

In some embodiments, the IL-15 molecule is a variant of human IL-5, e.g., having one or more amino acid alterations, e.g., substitutions, to the human IL-15 amino acid sequence. In some embodiments, the IL-15 variant comprises, or consists of, a mutation at position 45, 51, 52, or 72, e.g., as described in US 2016/0184399. In some embodiments, the IL-15 variant comprises, or consists of, an N, S or L to one of D, E, A, Y or P substitution. In some embodiments, the mutation is chosen from L45D, L45E, S51D, L52D, N72D, N72E, N72A, N72S, N72Y, or N72P (in reference to the sequence of human IL-15, SEQ ID NO: 2).

In embodiments, the IL-15 molecule comprises an IL-15 variant, e.g., a human IL-15 polypeptide having one or more amino acid substitutions. In some embodiments, the IL-15 molecule comprises a substitution at position 72, e.g., an N to D substitution. In one embodiment, the IL-15 molecule is an IL-15N72D polypeptide of SEQ ID NO: 2 or an amino acid sequence having at least 80%, 85%, 90%, 95%, 96%, 97%, 98%, or 99% identity thereto, which has IL-15Ra binding activity.

In some embodiments, the IL-15 is administered with, such as in a complex with or as a fusion, with an IL-15 Receptor alpha (IL15RA). IL15RA specifically binds IL-15 with very high affinity, and is capable of binding IL1-5 independent of other subunits. In some aspects, this property allows IL-15 to be produced by one cell, endocytosed by another cell, and then presented to a third cell. In some embodiments, the subject is administered IL-15/IL-15Ra. In some embodiments, the subject is administered with a IL-15/IL-15R fusion protein. In some embodiments, the subject is administered with a single-chain IL-15/IL-15R fusion protein. In some embodiments, the IL-15/IL-15Ra is a soluble IL 15Ra.IL15 complex (e.g. Mortier E et al., JBC 2006; Bessard A, Mol. Cancer Ther., 2009; and Desbois M, J. Immunol., 2016).

In some embodiments, the cytokine is IL-2 or a functional portion thereof. In some embodiments, IL-2 is a member of a cytokine family that also includes IL-4, IL-7, IL-9, IL-15 and IL-21. IL-2 signals through a receptor complex consisting of three chains, termed alpha, beta and gamma. The gamma chain is shared by all members of this family of cytokine receptors. IL-2, which similar to IL-15, facilitates production of immunoglobulins made by B cells and induces the differentiation and proliferation of NK cells. Primary differences between IL-2 and IL-15 are found in adaptive immune responses. For example, IL-2 is necessary for adaptive immunity to foreign pathogens, as it is the basis for the development of immunological memory. On the other hand, IL-15 is necessary for maintaining highly specific T cell responses by supporting the survival of CD8 memory T cells. All or a functional portion of IL-2 can be expressed as a membrane-bound polypeptide and/or as a secreted polypeptide. As will be appreciated by those of skill in the art, the sequence of a variety of IL-2 molecules are known in the art. In one aspect, the IL-2 is a wild type IL-2. In some aspects, the IL-2 is a mammalian IL-2. In some embodiments, the IL-2 is a human IL-2.

In some embodiments, the IL-2 is in a mature form lacking the signal peptide sequence and in some cases also the propeptide sequence. In some embodiments, the IL-2 has the sequence of amino acids set forth in SEQ ID NO:1 or a sequence that has at least or at least about 85%, at least or at least about 90%, at least or at least about 95%, or at least or at least about 98% sequence identity to SEQ ID NO:1.

In some embodiments, the cytokine is IL-21 or a functional portion thereof. IL-21 binds to the IL-21 receptor (IL-21 R) and co-receptor, the common gamma chain (CD 132), The IL-21 receptor has been identified on NK cells, T cells and B cell indicating IL-21 acts on hematopoietic lineage cells, in particular lymphoid progenitor cells and lymphoid cells. IL-21 has been shown to be a potent modulator of cytotoxic T cells and NK cells. (Parrish-Novak, et al. Nature 408:57-63, 2000; Parrish-Novak, et al., J. Leuk. Bio. 72:856-863, 202: Collins et al., Immunol. Res. 28:131-140, 2003; Brady, et al. J. Immunol. 172:2048-58, 2004.) In murine studies, IL-21 potentiates the maturation and effector function of NK cells (Kasaian et al., Immunity 16:559-569, 2002).

As will be appreciated by those of skill in the art, the sequence of a variety of IL-21 molecules are known in the art. In one aspect, the IL-21 is a wild type IL-21. In some aspects, the IL-21 is a mammalian IL-21. In an embodiment, the IL-21 sequence is a human IL-21 sequence. Human IL-21 amino acid sequences include, for example, Genbank Accession Nos: AAU88182.1, EAX05226.1, CAI94500.1, CAJ47524.1, CAL81203.1, CAN87399.1, CAS03522.1, CAV33288.1, CBE74752.1, CBI70418.1, CBI85469.1, CBI85472.1, CBL93962.1, CCA63962.1, AAG29348.1, AAH66258.1, AAH66259.1, AAH66260.1, AAH66261.1, AAH66262.1, AAH69124.1, and ABG36529.1.

In some embodiments, the IL-21 is in a mature form lacking the signal peptide sequence and in some cases also the propeptide sequence. In some embodiments, the IL-21 has the sequence of amino acids set forth in SEQ ID NO:3 or a sequence that has at least or at least about 85%, at least or at least about 90%, at least or at least about 95%, or at least or at least about 98% sequence identity to SEQ ID NO:3. In some embodiments, the IL-21 has the sequence of amino acids set forth in SEQ ID NO:4 or a sequence that has at least or at least about 85%, at least or at least about 90%, at least or at least about 95%, or at least or at least about 98% sequence identity to SEQ ID NO:4.

The cytokine (e.g., IL-2, IL-15, or IL-21) amino acid sequences may comprise any functional portion of mature cytokine, e.g. any functional portion of a mature, IL-2, mature, IL-15 or mature IL-15. The functional portion can be any portion comprising contiguous amino acids of the interleukin of which it is a part, provided that the functional portion specifically binds to the respective interleukin receptor. The term “functional portion” when used in reference to an interleukin refers to any part or fragment of the interleukin, which part or fragment retains the biological activity of the interleukin of which it is a part (the parent interleukin). Functional portions encompass, for example, those parts of an interleukin that retain the ability to specifically bind to the respective interleukin receptor, activate the downstream targets of the interleukin, and/or induce one or more of the differentiation, proliferation (or death) and activity of immune cells, e.g., NK cells, to a similar extent, the same extent, or to a higher extent, as the parent interleukin. The biological activity of the functional portion of the interleukin may be measured using assays known in the art. In reference to the parent interleukin, the functional portion can comprise, for instance, about 60%, about 70%, about 80%, about 90%, about 95%, or more, of the amino acid sequence of the parent mature interleukin.

Included in the scope of the cytokine or functional portion in accord with the provided embodiments are functional variants of the interleukins described herein. The term “functional variant” as used herein refers to an interleukin having substantial or significant sequence identity or similarity to a parent interleukin, which functional variant retains the biological activity of the interleukin of which it is a variant. Functional variants encompass, for example, those variants of the interleukin described herein (the parent interleukin) that retain the ability to specifically bind to the respective interleukin receptor, activate the downstream targets of the interleukin, and/or induce one or more of the differentiation, proliferation (or death) and activity of immune cells, e.g., NK cells, to a similar extent, the same extent, or to a higher extent, as the parent interleukin. In reference to the parent interleukin, the functional variant can, for instance, be at least about 80%, about 90%, about 95%, about 99% or more identical in amino acid sequence to the parent interleukin.

A functional variant can, for example, comprise the amino acid sequence of the parent interleukin with at least one conservative amino acid substitution. Alternatively or additionally, the functional variants can comprise the amino acid sequence of the parent interleukin with at least one non-conservative amino acid substitution. In some embodiments, the amino acid substitution, e.g. conservative or non-conservative amino acid substitution, does not interfere with or inhibit the biological activity of the functional variant as compared to the parental interleukin sequence. In some embodiments, the amino acid substitution, e.g. conservative or non-conservative amino acid substitution, may enhance the biological activity of the functional variant, such that the biological activity of the functional variant is increased as compared to the parent interleukin.

In some embodiments, the amino acid substitution(s) of the interleukin are conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, lie, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gin, Ser, Thr, Tyr, etc.), an amino acid with a beta-branched side-chain substituted for another amino acid with a beta-branched side-chain (e.g., lie, Thr, and Val), an amino acid with an aromatic side-chain substituted for another amino acid with an aromatic side chain (e.g., His, Phe, Trp, and Tyr), etc.

In some embodiments, a dose of the provided engineered g-NK cell compositions and the cytokines or growth factors are administered sequentially. For example, the g-NK cells may be administered first, followed by administration of the cytokines and/or growth factors. In some embodiments, a dose of cells containing engineered g-NK cells are administered simultaneously with the cytokines or growth factors.

In some embodiments, the subject is administered one or more cytokines (such as IL-2, IL-15, IL-21, IL-27, and/or IL-12) to support survival and/or growth of NK cells. The cytokine(s) can be administered before, after, or substantially simultaneously with the NK cells. In some examples, the cytokine(s) can be administered after the NK cells. In one specific example, the cytokine(s) is administered to the subject within about 1-8 hours (such as within about 1-4 hours, about 2-6 hours, about 4-6 hours, or about 5-8 hours) of the administration of the NK cells.

### 3. Cytotoxic Agents of Lymphodepleting Therapy

In some embodiments, the provided methods also can include administering a dose of cells containing engineered g-NK cells with another treatment, such as with a chemotherapeutic agent or cytotoxic agent or other treatment.

In some aspects, the provided methods can further include administering one or more lymphodepleting therapies, such as prior to or simultaneous with initiation of administration of the g-NK cell composition containing engineered g-NK cells. In some embodiments, the lymphodepleting therapy comprises administration of a phosphamide, such as cyclophosphamide. In some embodiments, the lymphodepleting therapy can include administration of fludarabine.

In some aspects, preconditioning subjects with immunodepleting (e.g., lymphodepleting) therapies can improve the effects of adoptive cell therapy (ACT). In some embodiments, the lymphodepleting therapy includes combinations of cyclosporine and fludarabine.

Such preconditioning can be carried out with the goal of reducing the risk of one or more of various outcomes that could dampen efficacy of the therapy. These include the phenomenon known as “cytokine sink,” by which T cells, B cells, NK cells compete with TILs for homeostatic and activating cytokines, such as IL-2, IL-7, and/or IL-15; suppression of TILs by regulatory T cells, NK cells, or other cells of the immune system; impact of negative regulators in the tumor microenvironment. Muranski et al., Nat Clin Pract Oncol. December; 3(12): 668-681 (2006).

Thus in some embodiments, the provided method further involves administering a lymphodepleting therapy to the subject. In some embodiments, the method involves administering the lymphodepleting therapy to the subject prior to the administration of the dose of cells. In some embodiments, the lymphodepleting therapy contains a chemotherapeutic agent such as fludarabine and/or cyclophosphamide. In some embodiments, the administration of the cells and/or the lymphodepleting therapy is carried out via outpatient delivery.

In some embodiments, the methods include administering a preconditioning agent, such as a lymphodepleting or chemotherapeutic agent, such as cyclophosphamide, fludarabine, or combinations thereof to a subject prior to the administration of the dose of cells. For example, the subject may be administered a preconditioning agent, such as a lymphodepleting or chemotherapeutic agent, such as cyclophosphamide, fludarabine, or combinations thereof, at least 2 days prior, such as at least 3, 4, 5, 6, or 7 days prior, to the first or subsequent dose. In some embodiments, the subject is administered a preconditioning agent, such as a lymphodepleting or chemotherapeutic agent, such as cyclophosphamide, fludarabine, or combinations thereof, no more than 7 days prior, such as no more than 6, 5, 4, 3, or 2 days prior, to the administration of the dose of cells. In some embodiments, the subject is administered a preconditioning agent, such as a lymphodepleting or chemotherapeutic agent, such as cyclophosphamide, fludarabine, or combinations thereof, no more than 14 days prior, such as no more than 13, 12, 11, 10, 9 or 8 days prior, to the administration of the dose of cells.

In some embodiments, the subject is preconditioned with cyclophosphamide at a dose between or between about 20 mg/kg and 100 mg/kg, such as between or between about 40 mg/kg and 80 mg/kg. In some aspects, the subject is preconditioned with or with about 60 mg/kg of cyclophosphamide. In some embodiments, the fludarabine can be administered in a single dose or can be administered in a plurality of doses, such as given daily, every other day or every three days. In some embodiments, the cyclophosphamide is administered once daily for one or two days.

In some embodiments, where the lymphodepleting agent comprises fludarabine, the subject is administered fludarabine at a dose between or between about 1 mg/m² and 100 mg/m², such as between or between about 10 mg/m² and 75 mg/m², 15 mg/m² and 50 mg/m², 20 mg/m² and 30 mg/m², or 24 mg/m² and 26 mg/m². In some instances, the subject is administered 25 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered 60 mg/kg (˜2 g/m²) of cyclophosphamide and 3 to 5 doses of 25 mg/m² fludarabine prior to the dose of cells.

In some embodiments, prior to the administration of the dose of g-NK cells, the subject has received a lymphodepleting therapy. In some embodiments, the lymphodepleting therapy includes fludarabine and/or cyclophosphamide. In some embodiments, the lymphodepleting includes the administration of fludarabine at or about 20-40 mg/m² body surface area of the subject, optionally at or about 30 mg/m², daily, for 2-4 days, and/or cyclophosphamide at or about 200-400 mg/m² body surface area of the subject, optionally at or about 300 mg/m², daily, for 2-4 days.

In some embodiments, the lymphodepleting therapy includes fludarabine and cyclophosphamide. In some embodiments, the lymphodepleting therapy includes the administration of fludarabine at or about 30 mg/m² body surface area of the subject, daily, and cyclophosphamide at or about 300 mg/m² body surface area of the subject, daily, each for 2-4 days, optionally 3 days.

In some embodiments, the administration of the preconditioning agent prior to infusion of the dose of cells improves an outcome of the treatment. For example, in some aspects, preconditioning, such as a lymphodepleting or chemotherapeutic agent, such as cyclophosphamide, fludarabine, or combinations thereof, improves the efficacy of treatment with the dose or increases the persistence of the NK cells in the subject. In some embodiments, preconditioning treatment increases disease-free survival, such as the percent of subjects that are alive and exhibit no minimal residual or molecularly detectable disease after a given period of time following the dose of cells. In some embodiments, the time to median disease-free survival is increased.

Once the cells are administered to the subject (e.g., human), the biological activity of the engineered cell populations in some aspects is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the NK cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines or other effector molecules, such as CD107a, IFNγ, and TNF. In some aspects the biological activity is measured by assessing clinical outcome, such as reduction in tumor burden or load. In some aspects, toxic outcomes, persistence and/or expansion of the cells, and/or presence or absence of a host immune response, are assessed.

### VIII. EXEMPLARY EMBODIMENTS

Among the provided embodiments are:
1. An engineered Natural Killer (NK) cell that is deficient in expression of FcRγ chain (g-NK cells) and comprises a heterologous nucleic acid encoding a chimeric antigen receptor (CAR). 1%
2. The engineered NK cell of embodiment 1, wherein the CAR comprises 1) an antigen binding domain; 2) a flexible linker; 3) a transmembrane region; and 4) an intracellular signaling domain.
3. The engineered NK cell of embodiment 2, wherein the antigen binding domain is targeted against a tumor antigen.
4. The engineered NK cell of embodiment 2 or 3, wherein the antigen binding domain is a single chain variable fragment (scFv).
5. The engineered NK cell of any of embodiments 2-4, wherein the intracellular signaling domain comprises a primary signaling domain and a costimulatory signaling domain.
6. The engineered NK cell of any of embodiments 2-5, wherein the intracellular signaling domain comprises one or more signaling domains of CD3ζ, DAP10, DAP12, CD28, 4-1BB, or OX40.
7. The engineered NK cell of any of embodiments 2-5, wherein the intracellular signaling domain comprises two or more signaling domains of CD3ζ, DAP10, DAP12, CD28, 4-1BB, or OX40.
8. The engineered NK cell of any of embodiments 1-7, wherein the heterologous nucleic acid encoding the CAR is stably integrated into the genome of the cell.
9. The engineered NK cell of any of embodiments 1-7, wherein the heterologous nucleic acid encoding the CAR is transiently expressed.
10. The engineered NK cell of any one of embodiments 1-9, wherein the g-NK cell has a surface phenotype that is CD16^{pos}/CD57^{pos}/CD7^{dim/neg}/CD161^{neg}.
11. The engineered NK cell of any of embodiments 1-10, wherein the g-NK cell further has a surface phenotype that is NKG2A^{neg}/CD161^{neg}.
12. The engineered NK cell of any of embodiments 1-11, wherein the g-NK cell further is CD38^{neg}.
13. The engineered NK cell of any of embodiments 1-12, wherein the g-NK cell has a surface phenotype that further is CD45^{pos}/CD3^{neg}/CD56^{pos}.
14. The engineered NK cell of any of embodiments 1-13, wherein the g-NK cells comprise CD16 158V/V (V158), 15. The engineered NK cell of any of embodiments 1-13, wherein the g-NK cells are CD16 158V/F.
16. A composition comprising a plurality of engineered g-NK cells of any of embodiments 1-15.
17. The composition of embodiment 16, wherein greater than at or about 50% of the NK cells or total cells in the composition are g-NK cells.
18. The composition of embodiment 16, wherein greater than at or about 60% of the NK cells or total cells in the composition are g-NK cells.
19. The composition of embodiment 16, wherein greater than at or about 70% of the NK cells or total cells in the composition are g-NK cells.
20. The composition of embodiment 16, wherein greater than at or about 80% of the NK cells or total cells in the composition are g-NK cells.
21. The composition of embodiment 16, wherein greater than at or about 90% of the NK cells or total cells in the composition are g-NK cells.
22. The composition of embodiment 16, wherein greater than at or about 95% of the NK cells or total cells in the composition are g-NK cells.
23. The composition of any one of embodiments 16-22, wherein the plurality of engineered g-NK cells comprises greater than at or about 20%, greater than at or about 30%, greater than at or about 40%, greater than at or about 50%, greater than at or about 60%, or greater than at or about 70% g-NK cells comprising a heterologous nucleic acid encoding the CAR.
24. The composition of any one of embodiments 16-23, wherein the total composition comprises greater than at or about 20%, greater than at or about 30%, greater than at or about 40%, greater than at or about 50%, greater than at or about 60%, or greater than at or about 70% g-NK cells comprising a heterologous nucleic acid encoding the CAR.
25. The composition of any one of embodiments 16-24, wherein greater than at or about 70% of the g-NK cells are positive for perforin and greater than at or about 70% of the g-NK cells are positive for granzyme B.
26. The composition of any one of embodiments 16-25, wherein greater than at or about 80% of the g-NK cells are positive for perforin and greater than at or about 80% of the g-NK cells are positive for granzyme B.
27. The composition of any one of embodiments 16-26, wherein greater than at or about 90% of the g-NK cells are positive for perforin and greater than at or about 90% of the g-NK cells are positive for granzyme B.
28. The composition of any one of embodiments 16-27, wherein greater than at or about 95% of the g-NK cells are positive for perforin and greater than at or about 95% of the g-NK cells are positive for granzyme B.
29. The composition of any of embodiments 25-28, wherein:
among the cells positive for perforin, the cells express a mean level of perforin as measured by intracellular flow cytometry that is, based on mean fluorescence intensity (MFI), at least at or about two times the mean level of perforin expressed by cells that are FcRγ^{pos}; and/or
among the cells positive for granzyme B, the cells express a mean level of granzyme B as measured by intracellular flow cytometry that is, based on mean fluorescence intensity (MFI), at least at or about two times the mean level of granzyme B expressed by cells that are FcRγ^{pos}.
30. The composition of any of embodiments 16-29, wherein greater than 10% of the cells in the composition are capable of degranulation against tumor target cells, optionally as measured by CD107a expression, optionally wherein the degranulation is measured in the absence of an antibody against the tumor target cells.
31. The composition of any of embodiments 16-30, wherein greater than 10% of the cells in the composition are capable of producing interferon-gamma or TNF-alpha against tumor target cells, optionally wherein the interferon-gamma or TNF-alpha is measured in the absence of an antibody against the tumor target cells.
32. The composition of any of embodiments 16-31, wherein, among the cells in the composition, greater than at or about 15%, greater than at or about 20%, greater than at or about 30%, greater than at or about 40% or greater than at or about 50% produce an effector cytokine in the presence of cells expressing a target antigen (target cells) and an antibody directed against the target antigen (anti-target antibody).
33. The composition of any of embodiments 16-32, wherein, among the total cells in the composition or among the g-NK cells in the composition, greater than at or about 30% are positive for NKG2C (NKG2C^{pos}) and/or greater than at or about 50% are negative or low for NKG2A (NKG2A^{neg}).
34. The composition of any of embodiments 16-33, wherein, among the total cells in the composition or among the g-NK cells in the composition, greater than at or about 35% are positive for NKG2C (NKG2C^{pos}) and/or greater than at or about 60% are negative or low for NKG2A (NKG2A^{neg}).
35. The composition of any of embodiments 16-34, wherein, among the total cells in the composition or among the g-NK cells in the composition, greater than at or about 40% are positive for NKG2C (NKG2C^{pos}) and/or greater than at or about 70% are negative or low for NKG2A (NKG2A^{neg}).
36. The composition of any of embodiments 16-34, wherein, among the total cells in the composition or among the g-NK cells in the composition, greater than at or about 45% are positive for NKG2C (NKG2C^{pos}) and/or greater than at or about 80% are negative or low for NKG2A (NKG2A^{neg}).
37. The composition of any of embodiments 16-34, wherein, among the total cells in the composition or among the g-NK cells in the composition, greater than at or about 50% are positive for NKG2C (NKG2C^{pos}) and/or greater than at or about 85% are negative or low for NKG2A (NKG2A^{neg}).
38. The composition of any of embodiments 16-34, wherein, among the total cells in the composition or among the g-NK cells in the composition, greater than at or about 55% are positive for NKG2C (NKG2C^{pos}) and/or greater than at or about 90% are negative or low for NKG2A (NKG2A^{neg}).
39. The composition of any of embodiments 16-34, wherein, among the total cells in the composition, or among the g-NK cells in the composition, greater than at or about 60% are positive for NKG2C (NKG2C^{pos}) and/or greater than at or about 95% are negative or low for NKG2A (NKG2A^{neg}).
40. The composition of any of embodiments 16-39, wherein, among the total cells in the composition or among the g-NK cells in the composition, greater than at or about 50%, greater than at or about 60%, greater than at or about 70%, greater than at or about 80%, or greater than at or about 90% are CD38^{neg}.
41. The composition of any of embodiments 16-39, wherein, among the total cells in the composition or among the g-NK cells in the composition, greater than at or about 50%, greater than at or about 60%, greater than at or about 70%, greater than at or about 80%, or greater than at or about 90% are CD16^{pos}/CD57^{pos}/CD7^{dim/neg}/CD161^{neg}.
42. The composition of any of embodiments 16-39, wherein, among the total cells in the composition or among the g-NK cells in the composition, greater than at or about 50%, greater than at or about 60%, greater than at or about 70%, greater than at or about 80%, or greater than at or about 90% are NKG2A^{neg}/CD161^{neg}.
43. The composition of any of embodiments 16-42, wherein the plurality of g-NK cells are CD16 158V/V (V158).
44. The composition of any of embodiments 16-42, wherein the plurality of g-NK cells are CD16 158V/F.
45. The composition of any of embodiments 16-44, wherein the composition comprises at least or about at least 10⁸ cells.
46. The composition of any of embodiments 16-45, wherein the number of g-NK cells in the composition is from at or about 10⁸ to at or about 10¹² cells, from at or about 10⁸ to at or about 10¹¹ cells, from at or about 10⁸ to at or about 10¹⁰ cells, from at or about 10⁸ to at or about 10⁹ cells, from at or about 10⁹ cells to at or about 10¹² cells, from at or about 10⁹ to at or about 10¹¹ cells, from at or about 10⁹ to at or about 10¹⁰ cells, from at or about 10¹⁰ to at or about 10¹² cells, from at or about 10¹⁰ to at or about 10¹¹ cells, or from at or about 10¹¹ to at or about 10¹² cells.
47. The composition of any of embodiments 16-46, wherein the number of g-NK cells in the composition is or is about 5×10⁸ cells, is or is about 1×10⁹ cells, is or is about 5×10¹⁰ cells, or is or is about 1×10¹⁰ cells.
48. The composition of any of embodiments 16-47, wherein the volume of the composition is between at or about 50 mL and at or about 500 mL, optionally at or about 200 mL.
49. The composition of any of embodiments 16-48, wherein the cells in the composition are from a single donor subject that have been expanded from the same biological sample.
50. The composition of any of embodiments 16-49, wherein the composition is a pharmaceutical composition.
51. The composition of any of embodiments 16-50, comprising a pharmaceutically acceptable excipient.
52. The composition of any of embodiments 16-51, wherein the composition is formulated in a serum-free cryopreservation medium comprising a cryoprotectant.
53. The composition of embodiment 52, wherein the cryoprotectant is DMSO and the cryopreservation medium is 5% to 10% DMSO (v/v).
54. The composition of embodiment 53, wherein the cryoprotectant is or is about 10% DMSO (v/v).
55. The composition of any of embodiments 16-54 that is sterile.
56. A sterile bag, comprising the composition of any of embodiments 16-55.
57. The sterile bag of embodiment 56, wherein the bag is a cryopreservation-compatible bag.
58. A method of producing a genetically engineered g-NK cell, comprising introducing into an NK cell deficient in expression of FcRγ chain (g-NK cells) a heterologous nucleic acid encoding a chimeric antigen receptor (CAR).
59. The method of embodiment 58, wherein the CAR comprises 1) an antigen binding domain; 2) a flexible linker (spacer); 3) a transmembrane region; and 4) an intracellular signaling domain.
60. The method of embodiment 59, wherein the antigen binding domain is targeted against a tumor antigen.
61. The method of embodiment 59 or 60, wherein the antigen binding domain is a single chain variable fragment (scFv).
62. The method of any of embodiments 59-61, wherein the intracellular signaling domain comprises one or more signaling domain from CD3ζ, DAP10, DAP12, CD28, 4-1BB, or OX40.
63. The method of any of embodiments 59-62, wherein the intracellular signaling domain comprises two or more signaling domains from CD28, 4-1BB, or OX40.
64. The method of any of embodiments 58-63, wherein the heterologous nucleic acid encoding the CAR is introduced under conditions for stable integration into the genome of the g-NK cell.
65. The method of any of embodiments 58-64, wherein the heterologous nucleic acid encoding the CAR is comprised in a viral vector and is introduced into the g-NK cell by transduction.
66. The method of embodiment 65, wherein the viral vector is a lentiviral vector.
67. The method of any one of embodiments 58-63, wherein the nucleic acid encoding the CAR is introduced under conditions for transient expression in the g-NK cell.
68. The method of any one of embodiments 58-63 and 67, wherein the nucleic acid encoding the CAR is introduced by non-viral delivery.
69. The method of any of embodiments 58-63, 67 and 68, wherein the nucleic acid encoding the CAR is introduced to the g-NK cell via a lipid nanoparticle.
70. The method of any of embodiments 58-69, wherein the nucleic acid encoding the CAR is DNA.
71. The method of any of embodiments 58-63 and 67-79, wherein the nucleic acid encoding the CAR is RNA.
72. The method of embodiment 71, wherein the RNA is mRNA.
73. The method of embodiment 72, wherein the RNA is a self-amplifying mRNA.
74. The method of any of embodiments 58-63 and 67-73, wherein the nucleic acid is introduced to the g-NK cell via electroporation.
75. The method of any of embodiments 58-74, wherein the g-NK cell composition has been produced by ex vivo expansion of NK cells enriched from a biological sample from a subject that are either (i) negative or low for CD3 and positive for CD57 (CD3^{neg}CD57^{pos}) or (ii) negative or low for CD3 and positive for CD56 (CD3^{neg}CD56^{pos}), wherein the enriched NK cells are cultured with irradiated HLA-E+ feeder cells and one or more recombinant cytokines.
76. The method of embodiment 75, wherein the one or more recombinant cytokine is selected from an effective amount of SCF, GSK3i, FLT3, IL-2, IL-6, IL-7, IL-15, IL-12, IL-18, IL-21, IL-27, or combinations thereof.
77. The method of embodiment 75 or embodiment 76, wherein the culturing is performed in the presence of two or more recombinant cytokines, wherein at least one recombinant cytokine is interleukin (IL)-2 and at least one recombinant cytokine is IL-21.
78. The method of any of embodiments 58-77, wherein the introducing is carried out during a method for expanding FcRγ-deficient NK (g-NK) cells, said method comprising:
(A) obtaining a population of primary human cells enriched for natural killer (NK) cells, wherein the population enriched for NK cells is selected from a biological sample from a human subject; and
(B) culturing the population of engineered NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21;
wherein the introducing the nucleic acid encoding the CAR is performed after step (A) and prior to, during or after step (B), thereby producing a population of engineered NK cells; and
wherein the method produces an expanded population of engineered NK cells that are enriched in g-NK cells and comprise cells engineered with a CAR.
79. The method of any of embodiments 58-77, wherein the introducing is carried out during a method for expanding FcRγ-deficient NK cells (g-NK), said method comprising:
(A) obtaining a population of primary human cells enriched for natural killer (NK) cells, wherein the population enriched for NK cells is selected from a biological sample from a human subject;
(B) introducing into the population of enriched NK cells the nucleic acid encoding a CAR, thereby producing a population of engineered NK cells; and
(C) culturing the population of engineered NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21, thereby producing an expanded population of NK cells; and
wherein the method produces an expanded population of engineered NK cells that are enriched in g-NK cells and comprise cells engineered with a CAR.
80. The method of any of embodiments 58-77, wherein the introducing is carried out during a method for expanding FcRγ-deficient NK cells (g-NK), said method comprising:
(A) obtaining a population of primary human cells enriched for natural killer (NK) cells, wherein the population enriched for NK cells is selected from a biological sample from a human subject; and
(B) culturing the population of enriched NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21, thereby producing an expanded population of NK cells; and
(C) introducing into NK cells of the expanded population of NK cells the nucleic acid encoding a CAR,
wherein the method produces an expanded population of engineered NK cells that are enriched in g-NK cells and comprises cells engineered with a CAR.
81. The method of any of embodiments 58-77, wherein the introducing is carried out during a method for expanding FcRγ-deficient NK cells (g-NK), said method comprising:
(A) obtaining a population of primary human cells enriched for natural killer (NK) cells, wherein the population enriched for NK cells is selected from a biological sample from a human subject; and
(B) performing a first expansion comprising culturing the population of enriched NK cells in culture medium under conditions for expanding the NK cells to produce a first expanded population of NK cells;
(C) introducing into NK cells of the first expanded population of NK cells the nucleic acid encoding a CAR, thereby producing a population of engineered NK cells; and
(D) performing a second expansion comprising culturing the population of engineered NK cells under conditions for further expansion of the NK cells,
wherein the first expansion and/or second expansion comprise culturing the population of enriched NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21; and
wherein the method produces an expanded population of engineered NK cells that are enriched in g-NK cells and comprise cells engineered with a CAR.
82. The method of any of embodiments 75-81, wherein the population of primary human cells enriched for NK cells are obtained by selecting from a biological sample from a human subject cells that are either (i) negative or low for CD3 and positive for CD57 (CD3^{neg}CD57^{pos}) or (ii) negative or low for CD3 and positive for CD56 (CD3^{BO}9CD56^{pos}).
83. The method of embodiment 82, wherein:
the population enriched for NK cells are cells further selected for cells positive for NKG2C (NKG2C^{pos});
the population enriched for NK cells are cells further selected for cells negative or low for NKG2A (NKG2A^{neg}); or
the population enriched for NK cells are cells further selected for cells positive for NKG2C and negative or low for NKG2A (NKG2C^{pos}NKG2A^{neg}).
84. The method of any of embodiments 75-83, wherein the human subject is a subject in which at least at or about 20% of natural killer (NK) cells in a peripheral blood sample from the subject are positive for NKG2C (NKG2C^{pos}) and at least 70% of NK cells in the peripheral blood sample are negative or low for NKG2A (NKG2A^{neg}).
85. The method of any of embodiments 75-84, wherein the subject is CMV-seropositive.
86. The method of any of embodiments 75-85, wherein the percentage of g-NK cells among NK cells in the biological sample from the subject is greater than at or about 5%, greater than at or about 10% or greater than at or about 30%.
87. The method of any of embodiments 75-86, wherein the percentage of g-NK cells among the population of enriched NK cells is between at or about 20% and at or about 90%, is between at or about 40% and at or about 90% or is between at or about 60% and at or about 90%.
88. The method of any of embodiments 75-87, wherein the population enriched for NK cells are cells selected from the biological sample that are negative or low for CD3 and positive for CD57 (CD3^{neg}CD57^{pos}).
89. The method of any of embodiments 75-87, wherein the population enriched for NK cells are cells selected from the biological sample that are negative or low for CD3 and positive for CD56 (CD3^{neg}CD56^{pos}).
90. The method of any of embodiments 77-89, wherein the two or more recombinant cytokines further comprises an effective amount of SCF, GSK3i, FLT3, IL-6, IL-7, IL-15, IL-12, IL-18, IL-27, or combinations thereof.
91. The method of any of embodiments 75-89, wherein the recombinant cytokines are IL-21 and IL-2.
92. The method of any of embodiments 75-90, wherein the recombinant cytokines are IL-21, IL-2, and IL-15.
93. The method of any of embodiments 75-92, wherein recombinant IL-21 is added to the culture medium during at least a portion of the culturing at a concentration that is from at or about 10 ng/mL to at or about 100 ng/mL.
94. The method of any of embodiments 75-93, wherein recombinant IL-21 is added to the culture medium during at least a portion of the culturing at a concentration that is at or about 25 ng/mL.
95. The method of any of embodiments 75-94, wherein recombinant IL-2 is added to the culture medium during at least a portion of the culturing at a concentration that is from at or about 10 IU/mL to at or about 500 IU/mL.
96. The method of any of embodiments 75-95, wherein recombinant IL-2 is added to the culture medium during at least a portion of the culturing at a concentration that is at or about 100 IU/mL.
97. The method of any of embodiments 75-96, wherein recombinant IL-2 is added to the culture medium during at least a portion of the culturing at a concentration that is at or about 500 IU/mL.
98. The method of any of embodiments 75-90 and 92-97, wherein recombinant IL-15 is added to the culture medium during at least a portion of the culturing at a concentration that is from at or about 1 ng/mL to 50 ng/mL.
99. The method of any of embodiments 75-90 and 92-97, wherein recombinant IL-15 is added to the culture medium during at least a portion of the culturing at a concentration that is at or about 10 ng/mL.
100. The method of any of embodiments 75-99, wherein the recombinant cytokines are added to the culture medium beginning at or about the initiation of the culturing.
101. The method of any of embodiments 75-100, wherein the method further comprises exchanging the culture medium one or more times during the culturing, wherein at each exchange of the culture medium, fresh media containing the recombinant cytokines is added.
102. The method of embodiment 101, wherein the exchanging of the culture medium is carried out every two or three days for the duration of the culturing.
103. The method of embodiment 101 or embodiment 102, wherein exchanging the media is performed after a first expansion without media exchange for up to 5 days, optionally after a first expansion without media exchange for up to 5 days.
104. The method of any of embodiments 75-103, wherein the human subject has the CD16 158V/V NK cell genotype or the CD16 158V/F NK cell genotype, optionally wherein the biological sample is from a human subject selected for the CD16 158V/V NK cell genotype or the CD16 158V/F NK cell genotype.
105. The method of any of embodiments 75-104, wherein the biological sample is or comprises peripheral blood mononuclear cells (PBMCs), optionally is a blood sample, an apheresis sample or a leukapheresis sample.
106. The method of any of embodiments 75-105, wherein the HLA-E+ feeder cells are K562 cells transformed with HLA-E (K562-HLA-E).
107. The method of any of embodiments 75-106, wherein the HLA-E+ feeder cells are 221.AEH cells.
108. The method of any of embodiments 75-107, wherein the ratio of irradiated HLA-E+ feeder cells to enriched NK cells is between 1:1 and 5:1, inclusive is between 1:1 and 3:1, inclusive, optionally is or is about 2.5:1 or is or is about 2:1 or is about 1:1
109. The method of any of embodiments 75-108, wherein the recombinant cytokines added to the culture medium during at least a portion of the culturing are 500 IU/mL IL-2, 10 ng/mL IL-15, and 25 ng/mL IL-21.
110. The method of any of embodiments 75-109, wherein the population of enriched NK cells comprises between at or about 2.0×10⁵ enriched NK cells and at or about 5.0×10⁷ enriched NK cells, between at or about 1.0×10⁶ enriched NK cells and at or about 1.0×10⁸ enriched NK cells, between at or about 1.0×10⁷ enriched NK cells and at or about 5.0×10⁸ enriched NK cells, or between at or about 1.0×10⁷ enriched NK cells and at or about 1.0×10⁹ enriched NK cells, each inclusive, optionally wherein the population of enriched NK cells comprises at or about 1.0×10⁶ enriched NK cells.
111. The method of any of embodiments 75-110, wherein the population of enriched NK cells at the initiation of the culturing is at a concentration of between or between about 0.05×10⁶ enriched NK cells/mL and 1.0×10⁶ enriched NK cells/mL or between or between about 0.05×10⁶ enriched NK cells/mL and 0.5×10⁶ enriched NK cells/mL, optionally wherein the population of enriched NK cells at the initiation of the culturing comprises a concentration of or about 0.2×10⁶ enriched NK cells/mL.
112. The method of any of embodiments 75-111, wherein the culturing is carried out until a time at which the method achieves expansion of at least or at least about 2.50×10⁸ g-NK cells, at least or at least about 5.00×10⁸ g-NK cells, at least or at least about 1.0×10⁹ g-NK cells or at least or at least about 5.0×10⁹ g-NK cells.
113. The method of any of embodiments 75-112, wherein the culturing is carried out for or about or at least or at least about 5 days, 6 days, 7 days, 8 days, 9 days, 10 days, 11 days, 12 days, 13 days, 14 days, 15 days, 16 days, 17 days, 18 days, 19 days, 20 day, 21 days, 22 days, 23 days, 24 days or 25 days.
114. The method of any of embodiments 75-113, further comprising collecting the expanded population of engineered NK cells produced by the method.
115. The method of any of embodiments 75-114, further comprising formulating the expanded population of engineered NK cells in a pharmaceutically acceptable excipient.
116. The method of embodiment 115, further comprising formulating the expanded population of engineered NK cells with a serum-free cryopreservation medium comprising a cryoprotectant.
117. The method of embodiment 116, wherein the cryoprotectant is DMSO, optionally wherein the cryoprotectant is DMSO and the cryopreservation medium is 5% to 10% DMSO (v/v), optionally is or is about 10% DMSO (v/v).
118. The method of any of embodiments 75-117, wherein, among the expanded population of engineered NK cells produced by the method, greater than 50% of the population are FcRγ^{neg}, greater than 60% of the population are FcRγ^{neg}, greater than 70% of the population are FcRγ^{neg}, greater than 80% of the population are FcRγ^{neg}, greater than 90% of the population are FcRγ^{neg} or greater than 95% of the population are FcRγ^{neg}.
119. A composition comprising a plurality of engineered NK cells produced by the method of any one of embodiments 58-118.
120. A method of treating a disease or condition in a subject, comprising administering an effective amount of cells of the composition of any of embodiments 16-55 and 119 to an individual in need thereof.
121. The method of embodiment 120, wherein the disease or condition is selected from the group consisting of an inflammatory condition, an infection, or cancer.
122. The method of embodiment 120 or embodiment 121, wherein the disease or condition is a cancer and the cancer is leukemia, a lymphoma, or a myeloma.
123. The method of embodiment 120 or embodiment 121, wherein the disease or condition is a cancer and the cancer comprises a solid tumor.
124. The method of embodiment 123, wherein the cancer is selected from among an adenocarcinoma of the stomach or gastroesophageal junction, a bladder cancer, a breast cancer, a brain cancer, a cervical cancer, a colorectal cancer, an endocrine/neuroendocrine cancer, a head and neck cancer, a gastrointestinal stromal cancer, a giant cell tumor of the bone, a kidney cancer, a liver cancer, a lung cancer, a neuroblastoma, an ovarian epithelial/fallopian tube/primary peritoneal cancers, a pancreatic cancer, a prostate cancer, a skin cancer, and a soft tissue carcinoma.
125. The method of any of embodiments 120-124, wherein the composition is administered as a monotherapy.
126. The method of any of embodiments 120-125, comprising administering from at or about 1×10⁵ to at or about 50×10⁹ cells of the g-NK cell composition to the individual.
127. The method of any of embodiments 120-126, comprising administering from at or about from at or about 1×10⁸ cells to at or about 50×10⁹ NK cells of the g-NK cell composition, optionally at or about 5×10⁸ cells of the g-NK cell composition, at or about 5×10⁹ cells of the g-NK cell composition or at or about 10×10⁹ cells of the g-NK cell composition.
128. The method of any one of embodiments 120-127, further comprising administering exogenous cytokine support to facilitate expansion or persistence of the administered NK cells in vivo in the subject, optionally wherein the exogenous cytokine is or comprises IL-15.
129. The method of any of embodiments 120-128, wherein prior to the administration of the dose of g-NK cells, the subject has received a lymphodepleting therapy.
130. The method of embodiment 129, wherein the lymphodepleting therapy comprises fludarabine and/or cyclophosphamide.
131. The method of embodiment 129 or embodiment 130, wherein the lymphodepleting comprises the administration of fludarabine at or about 20-40 mg/m² body surface area of the subject, optionally at or about 30 mg/m², daily, for 2-4 days, and/or cyclophosphamide at or about 200-400 mg/m² body surface area of the subject, optionally at or about 300 mg/m², daily, for 2-4 days.
132. The method of any of embodiments 129-131, wherein the lymphodepleting therapy comprises fludarabine and cyclophosphamide.
133. The method of any of embodiments 129-132, wherein the lymphodepleting therapy comprises the administration of fludarabine at or about 30 mg/m² body surface area of the subject, daily, and cyclophosphamide at or about 300 mg/m² body surface area of the subject, daily, each for 2-4 days, optionally 3 days.
134. The method of any of embodiments 129-133, wherein administration of the cells is initiated within two weeks or at or about two weeks after initiation of the lymphodepleting therapy.
135. The method of any one of embodiments 120-169, wherein the individual is a human.
136. The method of any one of embodiments 120-135, wherein the NK cells in the composition are allogenic to the individual.
137. The method of any one of embodiments 120-136, wherein the NK cells in the composition are autologous to the subject.

Among the provided embodiments are:
1. An engineered Natural Killer (NK) cell that is deficient in expression of FcRγ chain (g-NK cells), and comprises a heterologous nucleic acid encoding an immunomodulator.
2. The engineered NK cell of embodiment 1, wherein the immunomodulator is an immunosuppressant.
3. The engineered NK cell of embodiment 1, wherein the immunomodulator is an immunoactivator.
4. The engineered NK cell of embodiment 1 or embodiment 3, wherein the immunomodulator is a cytokine.
5. The engineered NK cell of any of embodiments 1-4, wherein the cytokine is secretable from the engineered NK cell.
6. The engineered NK cell of embodiment 5, wherein the secretable cytokine is IL-2 or a biological portion thereof; IL-15 or a biological portion thereof; or IL-21 or a biological portion thereof; or combinations thereof.
7. The engineered NK cell of any of embodiments 1-5, wherein the cytokine is membrane-bound.
8. The engineered NK cell of embodiment 7, wherein the membrane-bound cytokine is membrane-bound IL-2 (mbIL-2); membrane-bound IL-15 (mbIL-15); membrane-bound IL-21 (mbIL-21); or combinations thereof.
9. The engineered NK cell of any of embodiments 1-8, wherein the heterologous nucleic acid encoding the immunomodulator is stably integrated into the genome of the cell.
10. The engineered NK cell of any of embodiments 1-8, wherein the heterologous nucleic acid encoding the immunomodulator is transiently expressed.
11. The engineered NK cell of any one of embodiments 1-10, wherein the g-NK cell has a surface phenotype that is CD16^{pos}/CD57^{pos}/CD7^{dim/neg}/CD161^{neg}.
12. The engineered NK cell of any of embodiments 1-11, wherein the g-NK cell further has a surface phenotype that is NKG2A^{neg}/CD161^{neg}.
13. The engineered NK cell of any of embodiments 1-12, wherein the g-NK cell further is CD38^{neg}.
14. The engineered NK cell of any of embodiments 1-13, wherein the g-NK cell has a surface phenotype that further is CD45^{pos}/CD3^{neg}/CD56^{pos}.
15. The engineered NK cell of any of embodiments 1-14, wherein the g-NK cells comprises CD16 158V/V (V158).
16. The engineered NK cell of any of embodiments 1-14, wherein the g-NK cells are CD16 158V/F.
17. A composition comprising a plurality of engineered g-NK cells of any of embodiments 1-16.
18. The composition of embodiment 17, wherein greater than at or about 50% of the NK cells or total cells in the composition are g-NK cells.
19. The composition of embodiment 17, wherein greater than at or about 60% of the NK cells or total cells in the composition are g-NK cells.
20. The composition of embodiment 17, wherein greater than at or about 70% of the NK cells or total cells in the composition are g-NK cells.
21. The composition of embodiment 17, wherein greater than at or about 80% of the NK cells or total cells in the composition are g-NK cells.
22. The composition of embodiment 17, wherein greater than at or about 90% of the NK cells or total cells in the composition are g-NK cells.
23. The composition of embodiment 17, wherein greater than at or about 95% of the NK cells or total cells in the composition are g-NK cells.
24. The composition of any one of embodiments 17-23, wherein the plurality of engineered g-NK cells comprises greater than at or about 20%, greater than at or about 30%, greater than at or about 40%, greater than at or about 50%, greater than at or about 60%, or greater than at or about 70% g-NK cells comprising a heterologous nucleic acid encoding the immunomodulator.
25. The composition of any one of embodiments 17-24, wherein the total composition comprises greater than at or about 20%, greater than at or about 30%, greater than at or about 40%, greater than at or about 50%, greater than at or about 60%, or greater than at or about 70% g-NK cells comprising a heterologous nucleic acid encoding the immunomodulator.
26. The composition of any one of embodiments 17-25, wherein greater than at or about 70% of the g-NK cells are positive for perforin and greater than at or about 70% of the g-NK cells are positive for granzyme B.
27. The composition of any one of embodiments 17-26, wherein greater than at or about 80% of the g-NK cells are positive for perforin and greater than at or about 80% of the g-NK cells are positive for granzyme B.
28. The composition of any one of embodiments 17-27, wherein greater than at or about 90% of the g-NK cells are positive for perforin and greater than at or about 90% of the g-NK cells are positive for granzyme B.
29. The composition of any one of embodiments 17-28, wherein greater than at or about 95% of the g-NK cells are positive for perforin and greater than at or about 95% of the g-NK cells are positive for granzyme B.
30. The composition of any of embodiments 26-29, wherein:
among the cells positive for perforin, the cells express a mean level of perforin as measured by intracellular flow cytometry that is, based on mean fluorescence intensity (MFI), at least at or about two times the mean level of perforin expressed by cells that are FcRγ^{pos}; and/or
among the cells positive for granzyme B, the cells express a mean level of granzyme B as measured by intracellular flow cytometry that is, based on mean fluorescence intensity (MFI), at least at or about two times the mean level of granzyme B expressed by cells that are FcRγ^{pos}.
31. The composition of any of embodiments 17-30, wherein greater than 10% of the cells in the composition are capable of degranulation against tumor target cells, optionally as measured by CD107a expression, optionally wherein the degranulation is measured in the absence of an antibody against the tumor target cells.
32. The composition of any of embodiments 17-31, wherein greater than 10% of the cells in the composition are capable of producing interferon-gamma or TNF-alpha against tumor target cells, optionally wherein the interferon-gamma or TNF-alpha is measured in the absence of an antibody against the tumor target cells.
33. The composition of any of embodiments 17-32, wherein, among the cells in the composition, greater than at or about 15%, greater than at or about 20%, greater than at or about 30%, greater than at or about 40% or greater than at or about 50% produce an effector cytokine in the presence of cells expressing a target antigen (target cells) and an antibody directed against the target antigen (anti-target antibody).
34. The composition of any of embodiments 17-33, wherein, among the total cells in the composition or among the g-NK cells in the composition, greater than at or about 30% are positive for NKG2C (NKG2C^{pos}) and/or greater than at or about 50% are negative or low for NKG2A (NKG2A^{neg}).
35. The composition of any of embodiments 17-33, wherein, among the total cells in the composition or among the g-NK cells in the composition, greater than at or about 35% are positive for NKG2C (NKG2C^{pos}) and/or greater than at or about 60% are negative or low for NKG2A (NKG2A^{neg}).
36. The composition of any of embodiments 17-33, wherein, among the total cells in the composition or among the g-NK cells in the composition, greater than at or about 40% are positive for NKG2C (NKG2C^{pos}) and/or greater than at or about 70% are negative or low for NKG2A (NKG2A^{neg}).
37. The composition of any of embodiments 17-33, wherein, among the total cells in the composition or among the g-NK cells in the composition, greater than at or about 45% are positive for NKG2C (NKG2C^{pos}) and/or greater than at or about 80% are negative or low for NKG2A (NKG2A^{neg}).
38. The composition of any of embodiments 17-33, wherein, among the total cells in the composition or among the g-NK cells in the composition, greater than at or about 50% are positive for NKG2C (NKG2C^{pos}) and/or greater than at or about 85% are negative or low for NKG2A (NKG2A^{neg}).
39. The composition of any of embodiments 17-33, wherein, among the total cells in the composition or among the g-NK cells in the composition, greater than at or about 55% are positive for NKG2C (NKG2C^{pos}) and/or greater than at or about 90% are negative or low for NKG2A (NKG2A^{neg}).
40. The composition of any of embodiments 17-33, wherein, among the total cells in the composition, or among the g-NK cells in the composition, greater than at or about 60% are positive for NKG2C (NKG2C^{pos}) and/or greater than at or about 95% are negative or low for NKG2A (NKG2A^{neg}).
41. The composition of any of embodiments 17-40, wherein, among the total cells in the composition or among the g-NK cells in the composition, greater than at or about 50%, greater than at or about 60%, greater than at or about 70%, greater than at or about 80%, or greater than at or about 90% are CD38^{neg}.
42. The composition of any of embodiments 17-40, wherein, among the total cells in the composition or among the g-NK cells in the composition, greater than at or about 50%, greater than at or about 60%, greater than at or about 70%, greater than at or about 80%, or greater than at or about 90% are CD16^{pos}/CD57^{pos}/CD7^{dim/neg}/CD161^{neg}.
43. The composition of any of embodiments 17-40, wherein, among the total cells in the composition or among the g-NK cells in the composition, greater than at or about 50%, greater than at or about 60%, greater than at or about 70%, greater than at or about 80%, or greater than at or about 90% are NKG2A^{neg}/CD161^{neg}.
44. The composition of any of embodiments 17-43, wherein the plurality of g-NK cells are CD16 158V/V (V158).
45. The composition of any of embodiments 17-44, wherein the plurality of g-NK cells are CD16 158V/F.
46. The composition of any of embodiments 17-45, wherein the composition comprises at least or about at least 10⁸ cells.
47. The composition of any of embodiments 17-46, wherein the number of g-NK cells in the composition is from at or about 10⁸ to at or about 10¹² cells, from at or about 10⁸ to at or about 10¹¹ cells, from at or about 10⁸ to at or about 10¹⁰ cells, from at or about 10⁸ to at or about 10⁹ cells, from at or about 10⁹ cells to at or about 10¹² cells, from at or about 10⁹ to at or about 10¹¹ cells, from at or about 10⁹ to at or about 10¹⁰ cells, from at or about 10¹⁰ to at or about 10¹² cells, from at or about 10¹⁰ to at or about 10¹¹ cells, or from at or about 10¹¹ to at or about 10¹² cells.
48. The composition of any of embodiments 17-47, wherein the number of g-NK cells in the composition is or is about 5×10⁸ cells, is or is about 1×10⁹ cells, is or is about 5×10¹⁰ cells, or is or is about 1×10¹⁰ cells.
49. The composition of any of embodiments 17-48, wherein the volume of the composition is between at or about 50 mL and at or about 500 mL, optionally at or about 200 mL.
50. The composition of any of embodiments 17-49, wherein the cells in the composition are from a single donor subject that have been expanded from the same biological sample.
51. The composition of any of embodiments 17-50, wherein the composition is a pharmaceutical composition.
52. The composition of any of embodiments 17-51, comprising a pharmaceutically acceptable excipient.
53. The composition of any of embodiments 17-52, wherein the composition is formulated in a serum-free cryopreservation medium comprising a cryoprotectant.
54. The composition of embodiment 53, wherein the cryoprotectant is DMSO and the cryopreservation medium is 5% to 10% DMSO (v/v).
55. The composition of embodiment 54, wherein the cryoprotectant is or is about 10% DMSO (v/v).
56. The composition of any of embodiments 17-55 that is sterile.
57. A sterile bag, comprising the composition of any of embodiments 17-56.
58. The sterile bag of embodiment 57, wherein the bag is a cryopreservation-compatible bag.
59. A method of producing a genetically engineered g-NK cell, comprising introducing into the g-NK cell a heterologous nucleic acid encoding an immunomodulator, thereby producing a genetically engineered g-NK cell.
60. The method of embodiment 59, wherein the immunomodulator is an immunosuppressant.
61. The method of embodiment 59, wherein the immunomodulator is an immunoactivator.
62. The method of embodiment 59 or embodiment 61, wherein the immunomodulator is a cytokine.
63. The method of embodiment 62, wherein the cytokine is secretable from the engineered NK cell.
64. The method of embodiment 63, wherein the secretable cytokine is IL-2 or a biologically active portion thereof; IL-15 or a biologically active portion thereof; IL-21 or a biologically active portion thereof or combinations thereof.
65. The method of embodiment 62, wherein the cytokine is membrane bound.
66. The method of embodiment 65, wherein the membrane-bound cytokine is membrane bound IL-2 (mbIL-2); membrane-bound IL-15 (mbIL-15); or membrane bound IL-21 (mbIL-21) or combinations thereof.
67. The method of any one of embodiments 59-66, wherein the nucleic acid encoding the immunomodulator is introduced under conditions for stable integration into the genome of the g-NK cell.
68. The method of any of embodiments 59-67, wherein the nucleic acid encoding an immunomodulator is comprised in a viral vector and is introduced into the g-NK cell by transduction.
69. The method of embodiment 68, wherein the viral vector is a lentiviral vector.
70. The method of any one of embodiments 59-66, wherein the nucleic acid encoding the immunomodulator is introduced under conditions for transient expression in the g-NK cell.
71. The method of any one of embodiments 59-66 and 70, wherein the nucleic acid encoding the immunomodulator is introduced by non-viral delivery.
72. The method of any of embodiments 59-66, 70 and 71, wherein the nucleic acid encoding the immunomodulator is introduced to the g-NK cell via a lipid nanoparticle.
73. The method of any of embodiments 67-72, wherein the nucleic acid encoding the immunomodulator is DNA.
74. The method of any of embodiments 70-73, wherein the nucleic acid encoding the immunomodulator is RNA.
75. The method of embodiment 74, wherein the RNA is mRNA.
76. The method of embodiment 75, wherein the RNA is a self-amplifying mRNA.
77. The method of any of embodiments 59-66 and 70-75, wherein the nucleic acid encoding the immunomodulator is introduced to the g-NK cell via electroporation.
78. The method of any of embodiments 59-77, wherein the g-NK cell composition has been produced by ex vivo expansion of NK cells enriched from a biological sample from a subject that are either (i) negative or low for CD3 and positive for CD57 (CD3^{neg}CD57^{pos}) or (ii) negative or low for CD3 and positive for CD56 (CD3^{neg}CD56^{pos}), wherein the enriched NK cells cultured with irradiated HLA-E+ feeder cells and one or more recombinant cytokines.
79. The method of embodiment 78, wherein the one or more recombinant cytokine is selected from an effective amount of SCF, GSK3i, FLT3, IL-2, IL-6, IL-7, IL-15, IL-12, IL-18, IL-21, IL-27, or combinations thereof.
80. The method of embodiment 78 or embodiment 79, wherein the culturing is performed in the presence of two or more recombinant cytokines, wherein at least one recombinant cytokine is interleukin (IL)-2 and at least one recombinant cytokine is IL-21.
81. The method of any of embodiments 59-80, wherein the introducing is carried out during a method for expanding FcRγ-deficient NK (g-NK) cells, said method comprising:
(A) obtaining a population of primary human cells enriched for natural killer (NK) cells, wherein the population enriched for NK cells is selected from a biological sample from a human subject; and
(B) culturing the population of engineered NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21;
wherein the introducing the nucleic acid encoding the immunomodulator is performed after step (A) and prior to, during or after step (B), thereby producing a population of engineered NK cells; and
wherein the method produces an expanded population of engineered NK cells that are enriched in g-NK cells and comprises cells engineered with an immunomodulator.
82. The method of any of embodiments 59-80, wherein the introducing is carried out during a method for expanding FcRγ-deficient NK cells (g-NK), said method comprising:
(A) obtaining a population of primary human cells enriched for natural killer (NK) cells, wherein the population enriched for NK cells is selected from a biological sample from a human subject;
(B) introducing into the population of enriched NK cells the nucleic acid encoding an immunomodulator, thereby producing a population of engineered NK cells; and
(C) culturing the population of engineered NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21, thereby producing an expanded population of NK cells; and
wherein the method produces an expanded population of engineered NK cells that are enriched in g-NK cells and comprise cells engineered with an immunomodulator.
83. The method of any of embodiments 59-80, wherein the introducing is carried out during a method for expanding FcRγ-deficient NK cells (g-NK), said method comprising:
(A) obtaining a population of primary human cells enriched for natural killer (NK) cells, wherein the population enriched for NK cells is selected from a biological sample from a human subject; and
(B) culturing the population of enriched NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21, thereby producing an expanded population of NK cells; and
(C) introducing into NK cells of the expanded population of NK cells the nucleic acid encoding an immunomodulator,
wherein the method produces an expanded population of engineered NK cells that are enriched in g-NK cells and comprise cells engineered with an immunomodulator.
84. The method of any of embodiments 59-80, wherein the introducing is carried out during a method for expanding FcRγ-deficient NK cells (g-NK), said method comprising:
(A) obtaining a population of primary human cells enriched for natural killer (NK) cells, wherein the population enriched for NK cells is selected from a biological sample from a human subject; and
(B) performing a first expansion comprising culturing the population of enriched NK cells in culture medium under conditions for expanding the NK cells to produce a first expanded population of NK cells;
(C) introducing into NK cells of the first expanded population of NK cells (the nucleic acid encoding an immunomodulator, thereby producing a population of engineered NK cells; and
(D) performing a second expansion comprising culturing the population of engineered NK cells under conditions for further expansion of the NK cells,
wherein the first expansion and/or second expansion comprise culturing the population of enriched NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21; and
wherein the method produces an expanded population of engineered NK cells that are enriched in g-NK cells and comprise cells engineered with an immunomodulator.
85. The method of any of embodiments 78-84, wherein the population of primary human cells enriched for NK cells are obtained by selecting from a biological sample from a human subject cells that are either (i) negative or low for CD3 and positive for CD57 (CD3^{neg}CD57^{pos}) or (ii) negative or low for CD3 and positive for CD56 (CD3^{neg}CD56^{pos}).
86. The method of embodiment 85, wherein:
the population enriched for NK cells are cells further selected for cells positive for NKG2C (NKG2C^{pos});
the population enriched for NK cells are cells further selected for cells negative or low for NKG2A (NKG2A^{neg}); or
the population enriched for NK cells are cells further selected for cells positive for NKG2C and negative or low for NKG2A (NKG2C^{pos}NKG2A^{neg}).
87. The method of any of embodiments 78-86, wherein the human subject is a subject in which at least at or about 20% of natural killer (NK) cells in a peripheral blood sample from the subject are positive for NKG2C (NKG2C^{pos}) and at least 70% of NK cells in the peripheral blood sample are negative or low for NKG2A (NKG2A^{neg}).
88. The method of any of embodiments 78-87, wherein the subject is CMV-seropositive.
89. The method of any of embodiments 78-88, wherein the percentage of g-NK cells among NK cells in the biological sample from the subject is greater than at or about 5%, greater than at or about 10% or greater than at or about 30%.
90. The method of any of embodiments 78-89, wherein the percentage of g-NK cells among the population of enriched NK cells is between at or about 20% and at or about 90%, is between at or about 40% and at or about 90% or is between at or about 60% and at or about 90%.
91. The method of any of embodiments 78-90, wherein the population enriched for NK cells are cells selected from the biological sample that are negative or low for CD3 and positive for CD57 (CD3^{neg}CD57^{pos}).
92. The method of any of embodiments 78-90, wherein the population enriched for NK cells are cells selected from the biological sample that are negative or low for CD3 and positive for CD56 (CD3^{neg}CD56^{pos}).
93. The method of any of embodiments 80-92, wherein the two or more recombinant cytokines further comprises an effective amount of SCF, GSK3i, FLT3, IL-6, IL-7, IL-15, IL-12, IL-18, IL-27, or combinations thereof.
94. The method of any of embodiments 78-92, wherein the recombinant cytokines are IL-21 and IL-2.
95. The method of any of embodiments 78-93, wherein the recombinant cytokines are IL-21, IL-2, and IL-15.
96. The method of any of embodiments 78-95, wherein recombinant IL-21 is added to the culture medium during at least a portion of the culturing at a concentration that is from at or about 10 ng/mL to at or about 100 ng/mL.
97. The method of any of embodiments 78-96, wherein recombinant IL-21 is added to the culture medium during at least a portion of the culturing at a concentration that is at or about 25 ng/mL.
98. The method of any of embodiments 78-97, wherein recombinant IL-2 is added to the culture medium during at least a portion of the culturing at a concentration that is from at or about 10 IU/mL to at or about 500 IU/mL.
99. The method of any of embodiments 78-98, wherein recombinant IL-2 is added to the culture medium during at least a portion of the culturing at a concentration that is at or about 100 IU/mL.
100. The method of any of embodiments 78-99, wherein recombinant IL-2 is added to the culture medium during at least a portion of the culturing at a concentration that is at or about 500 IU/mL.
101. The method of any of embodiments 78-93 and 95-100, wherein recombinant IL-15 is added to the culture medium during at least a portion of the culturing at a concentration that is from at or about 1 ng/mL to 50 ng/mL.
102. The method of any of embodiments 78-93 and 95-101, wherein recombinant IL-15 is added to the culture medium during at least a portion of the culturing at a concentration that is at or about 10 ng/mL.
103. The method of any of embodiments 78-102, wherein the recombinant cytokines are added to the culture medium beginning at or about the initiation of the culturing.
104. The method of any of embodiments 78-103, wherein the method further comprises exchanging the culture medium one or more times during the culturing, wherein at each exchange of the culture medium, fresh media containing the recombinant cytokines is added.
105. The method of embodiment 104, wherein the exchanging of the culture medium is carried out every two or three days for the duration of the culturing.
106. The method of embodiment 104 or embodiment 105, wherein exchanging the media is performed after a first expansion without media exchange for up to 5 days, optionally after a first expansion without media exchange for up to 5 days.
107. The method of any of embodiments 78-106, wherein the human subject has the CD16 158V/V NK cell genotype or the CD16 158V/F NK cell genotype, optionally wherein the biological sample is from a human subject selected for the CD16 158V/V NK cell genotype or the CD16 158V/F NK cell genotype.
108. The method of any of embodiments 78-107, wherein the biological sample is or comprises peripheral blood mononuclear cells (PBMCs), optionally is a blood sample, an apheresis sample or a leukapheresis sample.
109. The method of any of embodiments 78-108, wherein the HLA-E+ feeder cells are K562 cells transformed with HLA-E (K562-HLA-E).
110. The method of any of embodiments 78-109, wherein the HLA-E+ feeder cells are 221.AEH cells.
111. The method of any of embodiments 78-110, wherein the ratio of irradiated HLA-E+ feeder cells to enriched NK cells is between 1:1 and 5:1, inclusive, is between 1:1 and 3:1, inclusive, optionally is or is about 2.5:1 or is or is about 2:1 or is about 1:1
112. The method of any of embodiments 78-111, wherein the recombinant cytokines added to the culture medium during at least a portion of the culturing are 500 IU/mL IL-2, 10 ng/mL IL-15, and 25 ng/mL IL-21.
113. The method of any of embodiments 78-112, wherein the population of enriched NK cells comprises between at or about 2.0×10⁵ enriched NK cells and at or about 5.0×10⁷ enriched NK cells, between at or about 1.0×10⁶ enriched NK cells and at or about 1.0×10⁸ enriched NK cells, between at or about 1.0×10⁷ enriched NK cells and at or about 5.0×10⁸ enriched NK cells, or between at or about 1.0×10⁷ enriched NK cells and at or about 1.0×10⁹ enriched NK cells, each inclusive, optionally wherein the population of enriched NK cells comprises at or about 1.0×10⁶ enriched NK cells.
114. The method of any of embodiments 78-113, wherein the population of enriched NK cells at the initiation of the culturing is at a concentration of between or between about 0.05×10⁶ enriched NK cells/mL and 1.0×10⁶ enriched NK cells/mL or between about 0.05×10⁶ enriched NK cells/mL and 0.5×10⁶ enriched NK cells/mL, optionally wherein the population of enriched NK cells at the initiation of the culturing comprises a concentration of or about 0.2×10⁶ enriched NK cells/mL.
115. The method of any of embodiments 78-114, wherein the culturing is carried out until a time at which the method achieves expansion of at least or at least about 2.50×10⁸ g-NK cells, at least or at least about 5.00×10⁸ g-NK cells, at least or at least about 1.0×10⁹ g-NK cells or at least or at least about 5.0×10⁹ g-NK cells.
116. The method of any of embodiments 78-115, wherein the culturing is carried out for or about or at least or at least about 5 days, 6 days, 7 days, 8 days, 9 days, 10 days, 11 days, 12 days, 13 days, 14 days, 15 days, 16 days, 17 days, 18 days, 19 days, 20 day, 21 days, 22 days, 23 days, 24 days or 25 days.
117. The method of any of embodiments 78-116, further comprising collecting the expanded population of engineered NK cells produced by the method.
118. The method of any of embodiments 78-117, further comprising formulating the expanded population of engineered NK cells in a pharmaceutically acceptable excipient.
119. The method of embodiment 118, further comprising formulating the expanded population of engineered NK cells with a serum-free cryopreservation medium comprising a cryoprotectant.
120. The method of embodiment 119, wherein the cryoprotectant is DMSO, optionally wherein the cryoprotectant is DMSO and the cryopreservation medium is 5% to 10% DMSO (v/v), optionally is or is about 10% DMSO (v/v).
121. The method of any of embodiments 78-120, wherein, among the expanded population of engineered NK cells produced by the method, greater than 50% of the population are FcRγ^{neg}, greater than 60% of the population are FcRγ^{neg}, greater than 70% of the population are FcRγ^{neg}, greater than 80% of the population are FcRγ^{neg}, greater than 90% of the population are FcRγ^{neg} or greater than 95% of the population are FcRγ^{neg}.
122. A composition comprising a plurality of engineered NK cells produced by the method of any one of embodiments 59-121.
123. A method of treating a disease or condition in a subject, comprising administering an effective amount of cells of the composition of any of embodiments 17-56 and 122 to an individual in need thereof.
124. The method of embodiment 123, wherein the disease or condition is selected from the group consisting of an inflammatory condition, an infection, or cancer.
125. The method of embodiment 123 or embodiment 124, wherein the disease or condition is a cancer and the cancer is leukemia, a lymphoma, or a myeloma.
126. The method of embodiment 123 or embodiment 124, wherein the disease or condition is a cancer and the cancer comprises a solid tumor.
127. The method of embodiments 126, wherein the cancer is selected from among an adenocarcinoma of the stomach or gastroesophageal junction, a bladder cancer, a breast cancer, a brain cancer, a cervical cancer, a colorectal cancer, an endocrine/neuroendocrine cancer, a head and neck cancer, a gastrointestinal stromal cancer, a giant cell tumor of the bone, a kidney cancer, a liver cancer, a lung cancer, a neuroblastoma, an ovarian epithelial/fallopian tube/primary peritoneal cancers, a pancreatic cancer, a prostate cancer, a skin cancer, and a soft tissue carcinoma.
128. The method of any of embodiments 123-127, wherein the composition is administered as a monotherapy.
129. The method of any of embodiments 123-127, further comprising administering an additional agent to the individual for treating the disease or condition.
130. The method of embodiment 129, wherein the additional agent is an antibody or an Fc-fusion protein.
131. The method of embodiment 130, wherein the disease or condition is a cancer and the additional agent, optional the antibody, recognizes a tumor antigen associated with the cancer.
132. The method of any of embodiments 123-131, comprising administering from at or about 1×10⁵ to at or about 50×10⁹ cells of the g-NK cell composition to the individual.
133. The method of any of embodiments 123-132, comprising administering from at or about 1×10⁸ cells to at or about 50×10⁹ NK cells of the g-NK cell composition, optionally at or about 5×10⁸ cells of the g-NK cell composition, at or about 5×10⁹ cells of the g-NK cell composition or at or about 10×10⁹ cells of the g-NK cell composition.
134. The method of any of embodiments 123-133, wherein prior to the administration of the dose of g-NK cells, the subject has received a lymphodepleting therapy.
135. The method of embodiment 134, wherein the lymphodepleting therapy comprises fludarabine and/or cyclophosphamide.
136 The method of embodiment 134 or embodiment 135, wherein the lymphodepleting comprises the administration of fludarabine at or about 20-40 mg/m² body surface area of the subject, optionally at or about 30 mg/m², daily, for 2-4 days, and/or cyclophosphamide at or about 200-400 mg/m² body surface area of the subject, optionally at or about 300 mg/m², daily, for 2-4 days.
137. The method of any of embodiments 134-136, wherein the lymphodepleting therapy comprises fludarabine and cyclophosphamide
138. The method of any of embodiments 134-137, wherein the lymphodepleting therapy comprises the administration of fludarabine at or about 30 mg/m² body surface area of the subject, daily, and cyclophosphamide at or about 300 mg/m² body surface area of the subject, daily, each for 2-4 days, optionally 3 days.
139. The method of any of embodiments 134-138, wherein administration of a dose of g-NK cells is initiated within two weeks or at or about two weeks after initiation of the lymphodepleting therapy.
140. The method of any one of embodiments 123-139, wherein the individual is a human.
141. The method of any one of embodiments 123-140, wherein the NK cells in the composition are allogenic to the individual.
142. The method of any one of embodiments 123-140, wherein the NK cells in the composition are autologous to the subject.

Among the provided embodiments are:
1. An engineered Natural Killer (NK) cell that is deficient in expression of FcRγ chain (g-NK cells), and comprises a heterologous nucleic acid encoding a chimeric antigen receptor (CAR) and a heterologous nucleic acid encoding an immunomodulator.
2. The engineered NK cell of embodiment 1, wherein the immunomodulator is an immunosuppressant.
3. The engineered NK cell of embodiment 1, wherein the immunomodulator is an immunoactivator.
4. The engineered NK cell of embodiment 1 or embodiment 3, wherein the immunomodulator is a cytokine.
5. The engineered NK cell of any of embodiments 1-4, wherein the cytokine is secretable from the engineered NK cell.
6. The engineered NK cell of embodiment 5, wherein the secretable cytokine is IL-2 or a biological portion thereof; IL-15 or a biological portion thereof; or IL-21 or a biological portion thereof; or combinations thereof.
7. The engineered NK cell of any of embodiments 1-5, wherein the cytokine is membrane-bound.
8. The engineered NK cell of embodiment 7, wherein the membrane-bound cytokine is membrane-bound IL-2 (mbIL-2); membrane-bound IL-15 (mbIL-15); membrane-bound IL-21 (mbIL-21); or combinations thereof.
9. The engineered NK cell of any of embodiments 1-8, wherein the CAR comprises 1) an antigen binding domain; 2) a flexible linker; 3) a transmembrane region; and 4) an intracellular signaling domain.
10. The engineered NK cell of embodiment 9, wherein the antigen binding domain is targeted against a tumor antigen.
11. The engineered NK cell of embodiment 9 or 10, wherein the antigen binding domain is a single chain variable fragment (scFv).
12. The engineered NK cell of any of embodiments 9-11, wherein the intracellular signaling domain comprises a primary signaling domain and a costimulatory signaling domain.
13. The engineered NK cell of any of embodiments 9-12, wherein the intracellular signaling domain comprises one or more signaling domains of CD3ζ, DAP10, DAP12, CD28, 4-1BB, or OX40.
14. The engineered NK cell of any of embodiments 9-12, wherein the intracellular signaling domain comprises two or more signaling domains of CD3ζ, DAP10, DAP12, CD28, 4-1BB, or OX40.
15. The engineered NK cell of any of embodiments 1-14, wherein the heterologous nucleic acid encoding the CAR is stably integrated into the genome of the cell.
16. The engineered NK cell of any of embodiments 1-14, wherein the heterologous nucleic acid encoding the CAR is transiently expressed.
17. The engineered NK cell of any of embodiments 1-16, wherein the heterologous nucleic acid encoding the immunomodulator is stably integrated into the genome of the cell.
18. The engineered NK cell of any of embodiments 1-16, wherein the heterologous nucleic acid encoding the immunomodulator is transiently expressed.
19. The engineered NK cell of any one of embodiments 1-18, wherein the g-NK cell has a surface phenotype that is CD16^{pos}/CD57^{pos}/CD7^{dim/neg}/CD161^{neg}.
20. The engineered NK cell of any of embodiments 1-19, wherein the g-NK cell further has a surface phenotype that is NKG2A^{neg}/CD161^{neg}.
21. The engineered NK cell of any of embodiments 1-20, wherein the g-NK cell further is CD38^{neg}.
22. The engineered NK cell of any of embodiments 1-21, wherein the g-NK cell has a surface phenotype that further is CD45^{pos}/CD3^{neg}/CD56^{pos}.
23. The engineered NK cell of any of embodiments 1-22, wherein the g-NK cells comprises CD16 158V/V (V158).
24. The engineered NK cell of any of embodiments 1-22, wherein the g-NK cells are CD16 158V/F.
25. A composition comprising a plurality of engineered g-NK cells of any of embodiments 1-24.
26. The composition of embodiment 25, wherein greater than at or about 50% of the NK cells or total cells in the composition are g-NK cells.
27. The composition of embodiment 25, wherein greater than at or about 60% of the NK cells or total cells in the composition are g-NK cells.
28. The composition of embodiment 25, wherein greater than at or about 70% of the NK cells or total cells in the composition are g-NK cells.
29. The composition of embodiment 25, wherein greater than at or about 80% of the NK cells or total cells in the composition are g-NK cells.
30. The composition of embodiment 25, wherein greater than at or about 90% of the NK cells or total cells in the composition are g-NK cells.
31. The composition of embodiment 25, wherein greater than at or about 95% of the NK cells or total cells in the composition are g-NK cells.
32. The composition of any one of embodiments 25-31, wherein the plurality of engineered g-NK cells comprises greater than at or about 20%, greater than at or about 30%, greater than at or about 40%, greater than at or about 50%, greater than at or about 60%, or greater than at or about 70% g-NK cells comprising a heterologous nucleic acid encoding the CAR and a heterologous nucleic acid encoding the immunomodulator.
33. The composition of any one of embodiments 25-32, wherein the total composition comprises greater than at or about 20%, greater than at or about 30%, greater than at or about 40%, greater than at or about 50%, greater than at or about 60%, or greater than at or about 70% g-NK cells comprising a heterologous nucleic acid encoding the CAR and a heterologous nucleic acid encoding the immunomodulator.
34. The composition of any one of embodiments 25-33, wherein the plurality of engineered g-NK cells comprises greater than at or about 20%, greater than at or about 30%, greater than at or about 40%, greater than at or about 50%, greater than at or about 60%, or greater than at or about 70% g-NK cells comprising a heterologous nucleic acid encoding the CAR
35. The composition of any one of embodiments 25-34, wherein the plurality of engineered g-NK cells comprises greater than at or about 20%, greater than at or about 30%, greater than at or about 40%, greater than at or about 50%, greater than at or about 60%, or greater than at or about 70% g-NK cells comprising a heterologous nucleic acid encoding the immunomodulator.
36. The composition of any one of embodiments 25-35, wherein the total composition comprises greater than at or about 20%, greater than at or about 30%, greater than at or about 40%, greater than at or about 50%, greater than at or about 60%, or greater than at or about 70% g-NK cells comprising a heterologous nucleic acid encoding the CAR
37. The composition of any one of embodiments 25-36, wherein the total composition comprises greater than at or about 20%, greater than at or about 30%, greater than at or about 40%, greater than at or about 50%, greater than at or about 60%, or greater than at or about 70% g-NK cells comprising a heterologous nucleic acid encoding the immunomodulator.
38. The composition of any one of embodiments 25-37, wherein greater than at or about 70% of the g-NK cells are positive for perforin and greater than at or about 70% of the g-NK cells are positive for granzyme B.
39. The composition of any one of embodiments 25-38, wherein greater than at or about 80% of the g-NK cells are positive for perforin and greater than at or about 80% of the g-NK cells are positive for granzyme B.
40. The composition of any one of embodiments 25-39, wherein greater than at or about 90% of the g-NK cells are positive for perforin and greater than at or about 90% of the g-NK cells are positive for granzyme B.
41. The composition of any one of embodiments 25-39, wherein greater than at or about 95% of the g-NK cells are positive for perforin and greater than at or about 95% of the g-NK cells are positive for granzyme B.
42. The composition of any of embodiments 38-41, wherein:
among the cells positive for perforin, the cells express a mean level of perforin as measured by intracellular flow cytometry that is, based on mean fluorescence intensity (MFI), at least at or about two times the mean level of perforin expressed by cells that are FcRγ^{pos}; and/or
among the cells positive for granzyme B, the cells express a mean level of granzyme B as measured by intracellular flow cytometry that is, based on mean fluorescence intensity (MFI), at least at or about two times the mean level of granzyme B expressed by cells that are FcRγ^{pos}.
43. The composition of any of embodiments 25-42, wherein greater than 10% of the cells in the composition are capable of degranulation against tumor target cells, optionally as measured by CD107a expression, optionally wherein the degranulation is measured in the absence of an antibody against the tumor target cells.
44. The composition of any of embodiments 25-43, wherein greater than 10% of the cells in the composition are capable of producing interferon-gamma or TNF-alpha against tumor target cells, optionally wherein the interferon-gamma or TNF-alpha is measured in the absence of an antibody against the tumor target cells.
45. The composition of any of embodiments 25-44, wherein, among the cells in the composition, greater than at or about 15%, greater than at or about 20%, greater than at or about 30%, greater than at or about 40% or greater than at or about 50% produce an effector cytokine in the presence of cells expressing a target antigen (target cells) and an antibody directed against the target antigen (anti-target antibody).
46. The composition of any of embodiments 25-45, wherein, among the total cells in the composition or among the g-NK cells in the composition, greater than at or about 30% are positive for NKG2C (NKG2C^{pos}) and/or greater than at or about 50% are negative or low for NKG2A (NKG2A^{neg}).
47. The composition of any of embodiments 25-45, wherein, among the total cells in the composition or among the g-NK cells in the composition, greater than at or about 35% are positive for NKG2C (NKG2C^{pos}) and/or greater than at or about 60% are negative or low for NKG2A (NKG2A^{neg}).
48. The composition of any of embodiments 25-45, wherein, among the total cells in the composition or among the g-NK cells in the composition, greater than at or about 40% are positive for NKG2C (NKG2C^{pos}) and/or greater than at or about 70% are negative or low for NKG2A (NKG2A^{neg}).
49. The composition of any of embodiments 25-45, wherein, among the total cells in the composition or among the g-NK cells in the composition, greater than at or about 45% are positive for NKG2C (NKG2C^{pos}) and/or greater than at or about 80% are negative or low for NKG2A (NKG2A^{neg}).
50. The composition of any of embodiments 25-45, wherein, among the total cells in the composition or among the g-NK cells in the composition, greater than at or about 50% are positive for NKG2C (NKG2C^{pos}) and/or greater than at or about 85% are negative or low for NKG2A (NKG2A^{neg}).
51. The composition of any of embodiments 25-45, wherein, among the total cells in the composition or among the g-NK cells in the composition, greater than at or about 55% are positive for NKG2C (NKG2C^{pos}) and/or greater than at or about 90% are negative or low for NKG2A (NKG2A^{neg}).
52. The composition of any of embodiments 25-45, wherein, among the total cells in the composition, or among the g-NK cells in the composition, greater than at or about 60% are positive for NKG2C (NKG2C^{pos}) and/or greater than at or about 95% are negative or low for NKG2A (NKG2A^{neg}).
53. The composition of any of embodiments 25-52, wherein, among the total cells in the composition or among the g-NK cells in the composition, greater than at or about 50%, greater than at or about 60%, greater than at or about 70%, greater than at or about 80%, or greater than at or about 90% are CD38^{neg}.
54. The composition of any of embodiments 25-52, wherein, among the total cells in the composition or among the g-NK cells in the composition, greater than at or about 50%, greater than at or about 60%, greater than at or about 70%, greater than at or about 80%, or greater than at or about 90% are CD16^{pos}/CD57^{pos}/CD7^{dim/neg}/CD161^{neg}.
55. The composition of any of embodiments 25-52, wherein, among the total cells in the composition or among the g-NK cells in the composition, greater than at or about 50%, greater than at or about 60%, greater than at or about 70%, greater than at or about 80%, or greater than at or about 90% are NKG2A^{neg}/CD161^{neg}.
56. The composition of any of embodiments 25-55, wherein the plurality of g-NK cells are CD16 158V/V (V158).
57. The composition of any of embodiments 25-56, wherein the plurality of g-NK cells are CD16 158V/F.
58. The composition of any of embodiments 25-57, wherein the immunomodulator is a cytokine, optionally comprising IL-2 or a biologically active portion thereof, IL-15 or a biologically active portion thereof, or IL-21 or a biologically active portion thereof.
59. The composition of embodiment 58, wherein the cytokine is secretable from the engineered cells of the composition or is membrane-bound.
60. The composition of any of embodiments 25-59, wherein the composition comprises at least or about at least 10⁸ cells.
61. The composition of any of embodiments 25-60, wherein the number of g-NK cells in the composition is from at or about 10⁸ to at or about 10¹² cells, from at or about 10⁸ to at or about 10¹¹ cells, from at or about 10⁸ to at or about 10¹⁰ cells, from at or about 10⁸ to at or about 10⁹ cells, from at or about 10⁹ cells to at or about 10¹² cells, from at or about 10⁹ to at or about 10¹¹ cells, from at or about 10⁹ to at or about 10¹⁰ cells, from at or about 10¹⁰ to at or about 10¹² cells, from at or about 10¹⁰ to at or about 10¹¹ cells, or from at or about 10¹¹ to at or about 10¹² cells.
62. The composition of any of embodiments 25-61, wherein the number of g-NK cells in the composition is or is about 5×10⁸ cells, is or is about 1×10⁹ cells, is or is about 5×10¹⁰ cells, or is or is about 1×10¹⁰ cells.
63. The composition of any of embodiments 25-62, wherein the volume of the composition is between at or about 50 mL and at or about 500 mL, optionally at or about 200 mL.
64. The composition of any of embodiments 25-63, wherein the cells in the composition are from a single donor subject that have been expanded from the same biological sample.
65. The composition of any of embodiments 25-64, wherein the composition is a pharmaceutical composition.
66. The composition of any of embodiments 25-65, comprising a pharmaceutically acceptable excipient.
67. The composition of any of embodiments 25-66, wherein the composition is formulated in a serum-free cryopreservation medium comprising a cryoprotectant.
68. The composition of embodiment 67, wherein the cryoprotectant is DMSO and the cryopreservation medium is 5% to 10% DMSO (v/v).
69. The composition of embodiment 68, wherein the cryoprotectant is or is about 10% DMSO (v/v).
70. The composition of any of embodiments 25-69 that is sterile.
71. A sterile bag, comprising the composition of any of embodiments 25-70.
72. The sterile bag of embodiment 71, wherein the bag is a cryopreservation-compatible bag.
73. A method of producing a genetically engineered g-NK cell, comprising:

(a) introducing into an NK cell deficient in expression of FcRγ chain (g-NK cells) a heterologous nucleic acid encoding a chimeric antigen receptor (CAR), and

(b) introducing into the g-NK cell a heterologous nucleic acid encoding an immunomodulator,

thereby producing a genetically engineered g-NK cell, wherein steps (a) and (b) are carried out simultaneously or sequentially in any order.
74. The method of embodiment 73, wherein the immunomodulator is an immunosuppressant.
75. The method of embodiment 73, wherein the immunomodulator is an immunoactivator.
76. The method of embodiment 73 or embodiment 75, wherein the immunomodulator is a cytokine.
77. The method of embodiment 76, wherein the cytokine is secretable from the engineered NK cell.
78. The method of embodiment 77, wherein the secretable cytokine is IL-2 or a biologically active portion thereof; IL-15 or a biologically active portion thereof; IL-21 or a biologically active portion thereof or combinations thereof.
79. The method of embodiment 76, wherein the cytokine is membrane bound.
80. The method of embodiment 79, wherein the membrane-bound cytokine is membrane bound IL-2 (mbIL-2); membrane-bound IL-15 (mbIL-15); or membrane bound IL-21 (mbIL-21) or combinations thereof.
81. The method of any of embodiments 73-80, wherein the CAR comprises 1) an antigen binding domain; 2) a flexible linker (spacer); 3) a transmembrane region; and 4) an intracellular signaling domain.
82. The method of embodiment 81, wherein the antigen binding domain is targeted against a tumor antigen.
83. The method of embodiment 81 or 82, wherein the antigen binding domain is a single chain variable fragment (scFv).
84. The method of any of embodiments 81-83, wherein the intracellular signaling domain comprises one or more signaling domain from CD3ζ, DAP10, DAP12, CD28, 4-1BB, or OX40.
85. The method of any of embodiments 81-84, wherein the intracellular signaling domain comprises two or more signaling domains from CD28, 4-1BB, or OX40.
86. The method of any of embodiments 73-85, wherein the heterologous nucleic acid encoding the CAR is introduced under conditions for stable integration into the genome of the g-NK cell.
87. The method of any of embodiments 73-86, wherein the heterologous nucleic acid encoding the CAR is comprised in a viral vector and is introduced into the g-NK cell by transduction
88. The method of embodiment 87, wherein the viral vector is a lentiviral vector.
89. The method of any one of embodiments 73-85, wherein the nucleic acid encoding the CAR is introduced under conditions for transient expression in the g-NK cell.
90. The method of any one of embodiments 73-85 and 89, wherein the nucleic acid encoding the CAR is introduced by non-viral delivery.
91. The method of any of embodiments 73-85, 89 and 90, wherein the nucleic acid encoding the CAR is introduced to the g-NK cell via a lipid nanoparticle.
92. The method of any of embodiments 73-91, wherein the nucleic acid encoding the CAR is DNA.
93. The method of any of embodiments 73-85 and 89-91, wherein the nucleic acid encoding the CAR is RNA.
94. The method of embodiment 93, wherein the RNA is mRNA.
95. The method of any of embodiments 73-85 and 89-94, wherein the nucleic acid is introduced to the g-NK cell via electroporation.
96. The method of any one of embodiments 73-95, wherein the nucleic acid encoding the immunomodulator is introduced under conditions for stable integration into the genome of the g-NK cell.
97. The method of any of embodiments 73-96, wherein the nucleic acid encoding an immunomodulator is comprised in a viral vector and is introduced into the g-NK cell by transduction.
98. The method of embodiment 97, wherein the viral vector is a lentiviral vector.
99. The method of any one of embodiments 73-95, wherein the nucleic acid encoding the immunomodulator is introduced under conditions for transient expression in the g-NK cell.
100. The method of any one of embodiments 73-95 and 99, wherein the nucleic acid encoding the immunomodulator is introduced by non-viral delivery.
101. The method of any of embodiments 73-95, 99 and 100, wherein the nucleic acid encoding the immunomodulator is introduced to the g-NK cell via a lipid nanoparticle.
102. The method of any of embodiments 96-101, wherein the nucleic acid encoding the immunomodulator is DNA.
103. The method of any of embodiments 99-101, wherein the nucleic acid encoding the immunomodulator is RNA.
104. The method of embodiment 103, wherein the RNA is mRNA.
105. The method of any of embodiments 73-85 and 99-104, wherein the nucleic acid encoding the immunomodulator is introduced to the g-NK cell via electroporation.
106. The method of embodiment 94 and embodiment 104, wherein the RNA is a self-amplifying mRNA.
107. The method of any of embodiments 73-106, wherein the nucleic acid encoding the CAR and the nucleic acid encoding the immunomodulator are encoded from the same polynucleotide and introduced together.
108. The method of embodiment 107, wherein the nucleic acid encoding the CAR and the nucleic acid encoding the immunomodulator are separated by a multicistronic element of the polynucleotide sequence.
109. The method of embodiment 108, wherein the multicistronic element is a self-cleaving peptide selected from the group consisting of T2A, P2A and F2A.
110. The method of any of embodiments 73-109, wherein the nucleic acid encoding the CAR and the nucleic acid encoding the immunomodulator are introduced simultaneously during an ex vivo process for expanding a population enriched in g-NK cells.
111. The method of any of embodiments 73-110, wherein the g-NK cell composition has been produced by ex vivo expansion of NK cells enriched from a biological sample from a subject that are either (i) negative or low for CD3 and positive for CD57 (CD3^{neg}CD57^{pos}) or (ii) negative or low for CD3 and positive for CD56 (CD3^{neg}CD56^{pos}), wherein the enriched NK cells are cultured with irradiated HLA-E+ feeder cells and one or more recombinant cytokines.
112. The method of embodiment 111, wherein the one or more recombinant cytokine is selected from an effective amount of SCF, GSK3i, FLT3, IL-2, IL-6, IL-7, IL-15, IL-12, IL-18, IL-21, IL-27, or combinations thereof.
113. The method of embodiment 111 or embodiment 112, wherein the culturing is performed in the presence of two or more recombinant cytokines, wherein at least one recombinant cytokine is interleukin (IL)-2 and at least one recombinant cytokine is IL-21.
114. The method of any of embodiments 73-113, wherein the introducing is carried out during a method for expanding FcRγ-deficient NK (g-NK) cells, said method comprising:
(A) obtaining a population of primary human cells enriched for natural killer (NK) cells, wherein the population enriched for NK cells is selected from a biological sample from a human subject; and
(B) culturing the population of engineered NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21;
wherein the introducing (i) the nucleic acid encoding the CAR and/or (ii) the nucleic acid encoding the immunomodulator is performed after step (A) and prior to, during or after step (B), wherein steps (i) and (ii) are carried out simultaneously or sequentially in any order, thereby producing a population of engineered NK cells; and
wherein the method produces an expanded population of engineered NK cells that are enriched in g-NK cells and comprise cells engineered with a CAR and an immunomodulator.
115. The method of any of embodiments 73-113, wherein the introducing is carried out during a method for expanding FcRγ-deficient NK cells (g-NK), said method comprising:
(A) obtaining a population of primary human cells enriched for natural killer (NK) cells, wherein the population enriched for NK cells is selected from a biological sample from a human subject;
(B) introducing into the population of enriched NK cells (i) the nucleic acid encoding a CAR, and (ii) the nucleic acid encoding an immunomodulator, wherein steps (i) and (ii) are carried out simultaneously or sequentially in any order, thereby producing a population of engineered NK cells; and
(C) culturing the population of engineered NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21, thereby producing an expanded population of NK cells; and
wherein the method produces an expanded population of engineered NK cells that are enriched in g-NK cells and comprise cells engineered with a CAR and an immunomodulator.
116. The method of any of embodiments 73-113, wherein the introducing is carried out during a method for expanding FcRγ-deficient NK cells (g-NK), said method comprising:
(A) obtaining a population of primary human cells enriched for natural killer (NK) cells, wherein the population enriched for NK cells is selected from a biological sample from a human subject; and
(B) culturing the population of enriched NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21, thereby producing an expanded population of NK cells; and
(C) introducing into NK cells of the expanded population of NK cells (i) the nucleic acid encoding a CAR, and (ii) the nucleic acid encoding an immunomodulator, wherein steps (i) and (ii) are carried out simultaneously or sequentially in any order,
wherein the method produces an expanded population of engineered NK cells that are enriched in g-NK cells and comprise cells engineered with a CAR and an immunomodulator.
117. The method of any of embodiments 73-113, wherein the introducing is carried out during a method for expanding FcRγ-deficient NK cells (g-NK), said method comprising:
(A) obtaining a population of primary human cells enriched for natural killer (NK) cells, wherein the population enriched for NK cells is selected from a biological sample from a human subject; and
(B) performing a first expansion comprising culturing the population of enriched NK cells in culture medium under conditions for expanding the NK cells to produce a first expanded population of NK cells;
(C) introducing into NK cells of the first expanded population of NK cells (i) the nucleic acid encoding a CAR, and (ii) the nucleic acid encoding an immunomodulator, wherein steps (i) and (ii) are carried out simultaneously or sequentially in any order, thereby producing a population of engineered NK cells; and
(D) performing a second expansion comprising culturing the population of engineered NK cells under conditions for further expansion of the NK cells,
wherein the first expansion and/or second expansion comprise culturing the population of enriched NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21; and
wherein the method produces an expanded population of engineered NK cells that are enriched in g-NK cells and comprise cells engineered with a CAR and an immunomodulator.
118. The method of any of embodiments 111-117, wherein the population of primary human cells enriched for NK cells are obtained by selecting from a biological sample from a human subject cells that are either (i) negative or low for CD3 and positive for CD57 (CD3^{neg}CD57^{pos}) or (ii) negative or low for CD3 and positive for CD56 (CD3^{neg}CD56^{pos}).
119. The method of embodiment 118, wherein:
the population enriched for NK cells are cells further selected for cells positive for NKG2C (NKG2C^{pos});
the population enriched for NK cells are cells further selected for cells negative or low for NKG2A (NKG2A^{neg}); or
the population enriched for NK cells are cells further selected for cells positive for NKG2C and negative or low for NKG2A (NKG2C^{pos}NKG2A^{neg}).
120. The method of any of embodiments 111-118, wherein the human subject is a subject in which at least at or about 20% of natural killer (NK) cells in a peripheral blood sample from the subject are positive for NKG2C (NKG2C^{pos}) and at least 70% of NK cells in the peripheral blood sample are negative or low for NKG2A (NKG2A^{neg}).
121. The method of any of embodiments 111-120, wherein the subject is CMV-seropositive.
122. The method of any of embodiments 111-121, wherein the percentage of g-NK cells among NK cells in the biological sample from the subject is greater than at or about 5%, greater than at or about 10% or greater than at or about 30%.
123. The method of any of embodiments 111-122, wherein the percentage of g-NK cells among the population of enriched NK cells is between at or about 20% and at or about 90%, is between at or about 40% and at or about 90% or is between at or about 60% and at or about 90%.
124. The method of any of embodiments 111-123, wherein the population enriched for NK cells are cells selected from the biological sample that are negative or low for CD3 and positive for CD57 (CD3^{neg}CD57^{pos}).
125. The method of any of embodiments 111-123, wherein the population enriched for NK cells are cells selected from the biological sample that are negative or low for CD3 and positive for CD56 (CD3^{neg}CD56^{pos}).
126. The method of any of embodiments 113-125, wherein the two or more recombinant cytokines further comprises an effective amount of SCF, GSK3i, FLT3, IL-6, IL-7, IL-15, IL-12, IL-18, IL-27, or combinations thereof.
127. The method of any of embodiments 111-125, wherein the recombinant cytokines are IL-21 and IL-2.
128. The method of any of embodiments 111-126, wherein the recombinant cytokines are IL-21, IL-2, and IL-15.
129. The method of any of embodiments 111-128, wherein recombinant IL-21 is added to the culture medium during at least a portion of the culturing at a concentration that is from at or about 10 ng/mL to at or about 100 ng/mL.
130. The method of any of embodiments 111-129, wherein recombinant IL-21 is added to the culture medium during at least a portion of the culturing at a concentration that is at or about 25 ng/mL.
131. The method of any of embodiments 111-130, wherein recombinant IL-2 is added to the culture medium during at least a portion of the culturing at a concentration that is from at or about 10 IU/mL to at or about 500 IU/mL.
132. The method of any of embodiments 111-131, wherein recombinant IL-2 is added to the culture medium during at least a portion of the culturing at a concentration that is at or about 100 IU/mL.
133. The method of any of embodiments 111-132, wherein recombinant IL-2 is added to the culture medium during at least a portion of the culturing at a concentration that is at or about 500 IU/mL.
134. The method of any of embodiments 111-126 and 128-134, wherein recombinant IL-15 is added to the culture medium during at least a portion of the culturing at a concentration that is from at or about 1 ng/mL to 50 ng/mL.
135. The method of any of embodiments 111-126 and 128-134, wherein recombinant IL-15 is added to the culture medium during at least a portion of the culturing at a concentration that is at or about 10 ng/mL.
136. The method of any of embodiments 111-135, wherein the recombinant cytokines are added to the culture medium beginning at or about the initiation of the culturing.
137. The method of any of embodiments 111-136, wherein the method further comprises exchanging the culture medium one or more times during the culturing, wherein at each exchange of the culture medium, fresh media containing the recombinant cytokines is added.
138. The method of embodiment 137, wherein the exchanging of the culture medium is carried out every two or three days for the duration of the culturing.
139. The method of embodiment 137 or embodiment 138, wherein exchanging the media is performed after a first expansion without media exchange for up to 5 days, optionally after a first expansion without media exchange for up to 5 days.
140. The method of any of embodiments 111-139, wherein the human subject has the CD16 158V/V NK cell genotype or the CD16 158V/F NK cell genotype, optionally wherein the biological sample is from a human subject selected for the CD16 158V/V NK cell genotype or the CD16 158V/F NK cell genotype.
141. The method of any of embodiments 111-140, wherein the biological sample is or comprises peripheral blood mononuclear cells (PBMCs), optionally is a blood sample, an apheresis sample or a leukapheresis sample.
142. The method of any of embodiments 111-141, wherein the HLA-E+ feeder cells are K562 cells transformed with HLA-E (K562-HLA-E).
143. The method of any of embodiments 111-145, wherein the HLA-E+ feeder cells are 221.AEH cells.
144. The method of any of embodiments 111-143, wherein the ratio of irradiated HLA-E+ feeder cells to enriched NK cells is between 1:1 and 5:1, inclusive, is between 1:1 and 3:1, inclusive, optionally is or is about 2.5:1 or is or is about 2:1 or is about 1:1
145. The method of any of embodiments 111-144, wherein the recombinant cytokines added to the culture medium during at least a portion of the culturing are 500 IU/mL IL-2, 10 ng/mL IL-15, and 25 ng/mL IL-21.
146. The method of any of embodiments 111-145, wherein the population of enriched NK cells comprises between at or about 2.0×10⁵ enriched NK cells and at or about 5.0×10⁷ enriched NK cells, between at or about 1.0×10⁶ enriched NK cells and at or about 1.0×10⁸ enriched NK cells, between at or about 1.0×10⁷ enriched NK cells and at or about 5.0×10⁸ enriched NK cells, or between at or about 1.0×10⁷ enriched NK cells and at or about 1.0×10⁸ enriched NK cells, each inclusive, optionally wherein the population of enriched NK cells comprises at or about 1.0×10⁶ enriched NK cells.
147. The method of any of embodiments 111-146, wherein the population of enriched NK cells at the initiation of the culturing is at a concentration of between or between about 0.05×10⁶ enriched NK cells/mL and 1.0×10⁶ enriched NK cells/mL or between about 0.05×10⁶ enriched NK cells/mL and 0.5×10⁶ enriched NK cells/mL, optionally wherein the population of enriched NK cells at the initiation of the culturing comprises a concentration of or about 0.2×10⁶ enriched NK cells/mL.
148. The method of any of embodiments 111-146, wherein the culturing is carried out until a time at which the method achieves expansion of at least or at least about 2.50×10⁸ g-NK cells, at least or at least about 5.00×10⁸ g-NK cells, at least or at least about 1.0×10⁹ g-NK cells or at least or at least about 5.0×10⁹ g-NK cells.
149. The method of any of embodiments 111-148, wherein the culturing is carried out for or about or at least or at least about 5 days, 6 days, 7 days, 8 days, 9 days, 10 days, 11 days, 12 days, 13 days, 14 days, 15 days, 16 days, 17 days, 18 days, 19 days, 20 day, 21 days, 22 days, 23 days, 24 days or 25 days.
150. The method of any of embodiments 111-149, further comprising collecting the expanded population of engineered NK cells produced by the method.
151. The method of any of embodiments 111-150, further comprising formulating the expanded population of engineered NK cells in a pharmaceutically acceptable excipient.
152. The method of embodiment 151, further comprising formulating the expanded population of engineered NK cells with a serum-free cryopreservation medium comprising a cryoprotectant.
153. The method of embodiment 152, wherein the cryoprotectant is DMSO, optionally wherein the cryoprotectant is DMSO and the cryopreservation medium is 5% to 10% DMSO (v/v), optionally is or is about 10% DMSO (v/v).
154. The method of any of embodiments 111-153, wherein, among the expanded population of engineered NK cells produced by the method, greater than 50% of the population are FcRγ^{neg}, greater than 60% of the population are FcRγ^{neg}, greater than 70% of the population are FcRγ^{neg}, greater than 80% of the population are FcRγ^{neg}, greater than 90% of the population are FcRγ^{neg} or greater than 95% of the population are FcRγ^{neg}.
155. A composition comprising a plurality of engineered NK cells produced by the method of any one of embodiments 73-154.
156. A method of treating a disease or condition in a subject, comprising administering an effective amount of cells of the composition of any of embodiments 25-72 and 155 to an individual in need thereof.
157. The method of embodiment 156, wherein the disease or condition is selected from the group consisting of an inflammatory condition, an infection, or cancer.
158. The method of embodiment 156 or embodiment 157, wherein the disease or condition is a cancer and the cancer is leukemia, a lymphoma, or a myeloma.
159. The method of embodiment 156 or embodiment 157, wherein the disease or condition is a cancer and the cancer comprises a solid tumor.
160. The method of embodiment 159, wherein the cancer is selected from among an adenocarcinoma of the stomach or gastroesophageal junction, a bladder cancer, a breast cancer, a brain cancer, a cervical cancer, a colorectal cancer, an endocrine/neuroendocrine cancer, a head and neck cancer, a gastrointestinal stromal cancer, a giant cell tumor of the bone, a kidney cancer, a liver cancer, a lung cancer, a neuroblastoma, an ovarian epithelial/fallopian tube/primary peritoneal cancers, a pancreatic cancer, a prostate cancer, a skin cancer, and a soft tissue carcinoma.
161. The method of any of embodiments 156-160, wherein the composition is administered as a monotherapy.
162. The method of any of embodiments 156-161, comprising administering from at or about 1×10⁵ to at or about 50×10⁹ cells of the g-NK cell composition to the individual.
163. The method of any of embodiments 156-162, comprising administering from at or about 1×10⁸ cells to at or about 50×10⁹ NK cells of the g-NK cell composition, optionally at or about 5×10⁸ cells of the g-NK cell composition, at or about 5×10⁹ cells of the g-NK cell composition or at or about 10×10⁹ cells of the g-NK cell composition.
164. The method of any of embodiments 156-163, wherein prior to the administration of the dose of g-NK cells, the subject has received a lymphodepleting therapy.
165. The method of embodiment 164, wherein the lymphodepleting therapy comprises fludarabine and/or cyclophosphamide.
166. The method of embodiment 164 or embodiment 165, wherein the lymphodepleting comprises the administration of fludarabine at or about 20-40 mg/m² body surface area of the subject, optionally at or about 30 mg/m², daily, for 2-4 days, and/or cyclophosphamide at or about 200-400 mg/m² body surface area of the subject, optionally at or about 300 mg/m², daily, for 2-4 days.
167. The method of any of embodiments 164-166, wherein the lymphodepleting therapy comprises fludarabine and cyclophosphamide.
168. The method of any of embodiments 164-167, wherein the lymphodepleting therapy comprises the administration of fludarabine at or about 30 mg/m² body surface area of the subject, daily, and cyclophosphamide at or about 300 mg/m² body surface area of the subject, daily, each for 2-4 days, optionally 3 days.
169. The method of any of embodiments 164-168, wherein administration of a dose of g-NK cells is initiated within two weeks or at or about two weeks after initiation of the lymphodepleting therapy.
170. The method of any one of embodiments 156-169, wherein the individual is a human.
171. The method of any one of embodiments 156-170, wherein the NK cells in the composition are allogenic to the individual.
172. The method of any one of embodiments 156-170, wherein the NK cells in the composition are autologous to the subject.

### IX. EXAMPLES

The following examples are included for illustrative purposes only and are not intended to limit the scope of the invention.

### Example 1: Expansion of g-NK Cells in the Presence of Different Cytokines

Fifty mL of fresh whole blood from a CMV-seropositive donor (NKG2C^{pos} and NKG2A^{neg} NK-cell percentages of 56.24% and 11.68%, respectively) was collected into ACD vacutainer tubes and diluted 1:1 with PBS. PBMCs were isolated by Histopaque® density centrifugation as per manufacturer's instructions. After harvesting the PBMC-containing buffy coat, the PBMCs were washed with PBS and counted. Following the cell count, a magnetic bead separation was conducted to increase the frequency of g-NK cells. The magnetic bead separation was a CD3 depletion followed by CD57 enrichment in order to isolate CD57^{pos} NK cells.

The transgenic lymphoma cell line 221.AEH (Lee et al. (1998) Journal of Immunology, 160:4951-4960) and the transgenic leukemia cell line K562-mb15-41BBL (Fujisaki et al. (2009) Cancer Research, 69(9): 4010-4017) were prepared as feeder cells for the NK cell expansion. Feeder cells were taken from fresh culture (i.e., not cryopreserved stock) and were irradiated prior to use. 221.AEH and K562-mb15-41BBL cells were expanded with a seeding density of 5×10⁵ cells per mL and a subculture density of 2×10⁵ cells per mL. The media used to grow the 221.AEH feeder cells was RPMI-1640 with 10% FBS and 200 μg/mL of Hygromycin B. The media used to grow the K562-mb15-41BBL feeder cells was RPMI-1640 with 10% FBS.

The non-cryopreserved NK cells were expanded under four different conditions: at a 2:1 AEH to NK cell ratio with 500 IU/mL IL-2; at a 2:1 K562-mb15-41BBL to NK cell ratio with 500 IU/mL IL-2; at a 1:1:1 AEH to K562-mb15-41BBL to NK cell ratio with 500 IU/mL IL-2; and at a 2:1 AEH to NK cell ratio with 500 IU/mL IL-2, 10 ng/mL IL-15, and 25 ng/mL IL-21. All expansions were carried out in CellGenix GMP SCGM media supplemented with 5% human AB Serum and with the respective cytokines. The co-cultured cells were cultivated for 21 days at 37° C. and 5% CO₂. Cells were counted every time the media was changed or replenished (day 5, 7, 10, 13, 16, 19, and 21), and the percentage of g-NK was assessed by flow cytometry at day 0, day 13, and day 21.

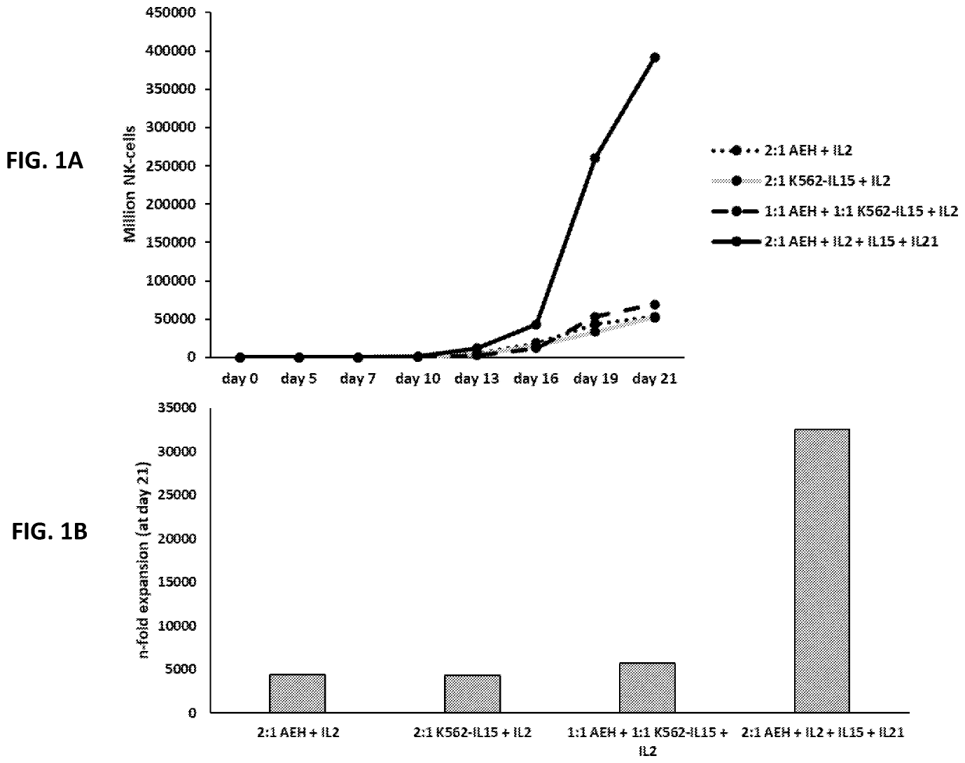

As shown in FIG. 1A-1B, the addition of IL-21 to the expansion media led to a marked increase in g-NK cell expansion. Total g-NK cell count (cells deficient in FcεR1γ, also referred to interchangeably herein FcRγ) was highest for g-NK cells expanded in the presence of IL-21 (FIG. 1A). Fold-expansion of g-NK cells by day 21 was also highest for g-NK cells expanded in the presence of IL-21 (FIG. 1B).

Together, these results show that the presence of IL-21 improves g-NK cell expansion.

### Example 2: Cell Effector Function of g-NK Cells Expanded in the Presence of Different Cytokines

In this study, NK cell effector function was measured in g-NK cells expanded in the presence of different feeder cells and cytokines, including in the presence of IL-21, as described in Example 1. Assays were performed as described below using target cell lines LP1 and MM.1S at a 0.5:1 NK to MM cell ratio and with antibodies daratumumab and elotuzumab.

### A. Cell Mediated Cytotoxicity

Upon thawing of expanded NK cells, 10⁴ NK cells were co-cultured with MM target cells at a 1:1 NK cell to MM cell ratio and in the presence of one μg/mL daratumumab (anti-CD38) or one μg/mL elotuzumab (anti-CD319). After a four-hour incubation at 37° C. in a CO₂ incubator, the cells were washed and stained with anti-CD3 and CD56 antibodies to quantify the number of NK cells. After a final wash, propidium iodide (PI) was added, and the number of NK cells, live target cells, and dead target cells were resolved using 4-color flow cytometry (Bigley et al. (2016), Clin. Exp. Immunol., 185:239-251).

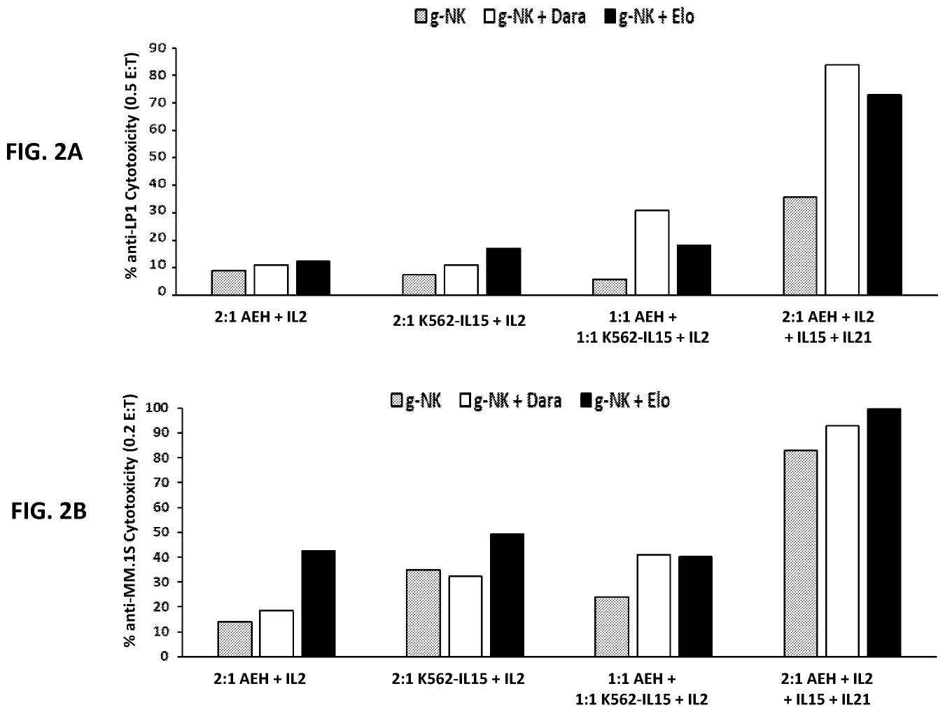

As shown in FIG. 2A-2B, g-NK cells expanded for 21 days in the presence of IL-21 had greater cell-mediated cytotoxicity against the CD38^{high} MM cell line LP1 (FIG. 2A) and the SLAMF7^{high} MM cell line MM.1S (FIG. 2B) than did g-NK cells expanded without IL-21. Greater cell-mediated cytotoxicity for IL-21 expanded g-NK cells was observed in the absence of antibody as well as in the presence of either daratumumab or elotuzumab.

Together, these results show that g-NK cells expanded in the presence of IL-21 have enhanced cell-mediated cytotoxicity against tumor cells compared to g-NK cells expanded without IL-21.

### B. Degranulation

Upon thawing of expanded NK cells, 2.0×10⁵ NK cells were co-cultured MM target cells at a 1:1 NK cell to MM cell ratio and in the presence of one μg/mL daratumumab or one μg/mL elotuzumab. For the degranulation assay, two μL of VioGreen-conjugated anti-CD107a was added to the co-culture for a one-hour incubation at 37° C. in a CO₂ incubator, after which four μL of BD GolgiStop containing monensin was added. For cytokine expression assays, six μL of BD GolgiStop containing brefeldin A was added instead. The cells were then incubated for an additional five hours at 37° C. in a CO2 incubator. Following incubation, the cells were harvested, washed, and stained with 0.5 μL of anti-CD45 antibody, 0.5 μL of anti-CD3 antibody, and one μL of anti-CD56 antibody (all antibodies purchased from Miltenyi Biotec). The cells were then fixed and permeabilized using the Inside Stain Kit from Miltenyi Biotec as per the manufacturer's instructions. The cells were then stained with one μL of anti-FcRγ, two μL of anti-perforin, two μL of anti-granzyme B, two μL of Interferon-gamma, and two μL of TNF-alpha antibodies, as described in Table E1. After a final wash, the cells were resolved using eight-color flow cytometry.

TABLE E1
Antibody Panel for Functional Assays.

| | | | | | | | | |
|---|---|---|---|---|---|---|---|---|
| Tubes | V1 | V2 | B1 | B2 | B3 | B4 | R1 | R2 |

Degranulation (release of cytotoxic granules)

| | | | | | | | |
|---|---|---|---|---|---|---|---|
| 1 | CD45 | CD107a | FcRγ | Perforin | CD3 | Granz B | CD56 |

Cytokine expression

| | | | | | | | |
|---|---|---|---|---|---|---|---|
| 2 | CD45 | CD107a | FcRγ | IFN-γ | CD3 | TNF-α | CD56 |

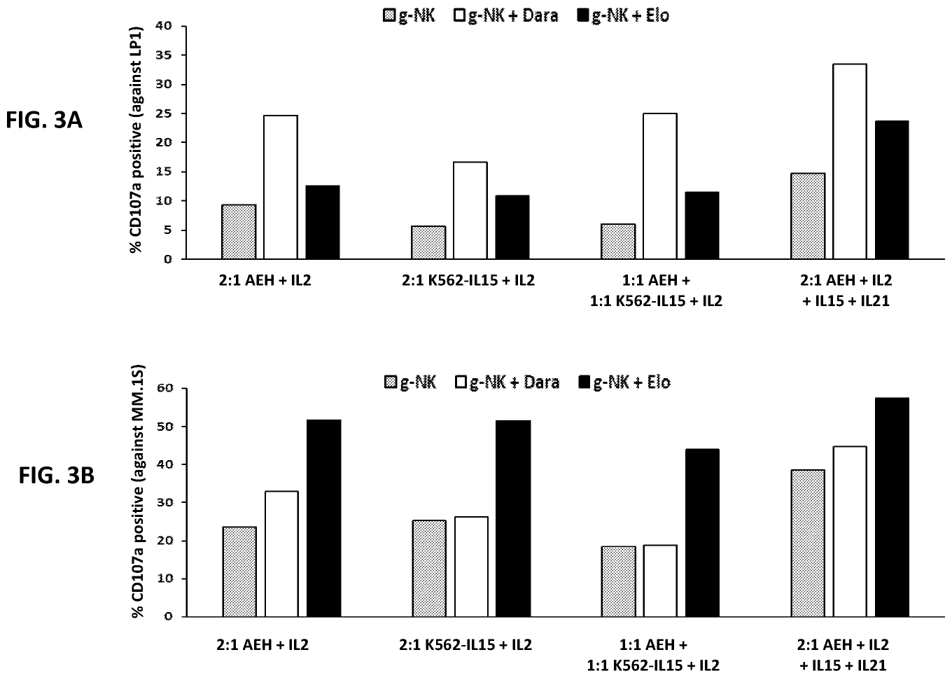

As shown in FIG. 3A-3D, after both 13 days (FIGS. 3A-3B) and 21 days (FIGS. 3C-3D) of expansion, g-NK cells expanded in the presence of IL-21 degranulated more against the CD38^{high} MM cell line LP1 (FIG. 3A and FIG. 3C) and the SLAMF7^{high} MM cell line MM.1S (FIG. 3B and FIG. 3D) than did g-NK cells expanded without IL-21. Greater degranulation for IL-21 expanded g-NK cells was observed in the absence of antibody as well as in the presence of either daratumumab or elotuzumab.

Together, these results show that g-NK cells expanded in the presence of IL-21 have enhanced degranulation against tumor cells compared to g-NK cells expanded without IL-21.

### C. Perforin and Granzyme B Expression

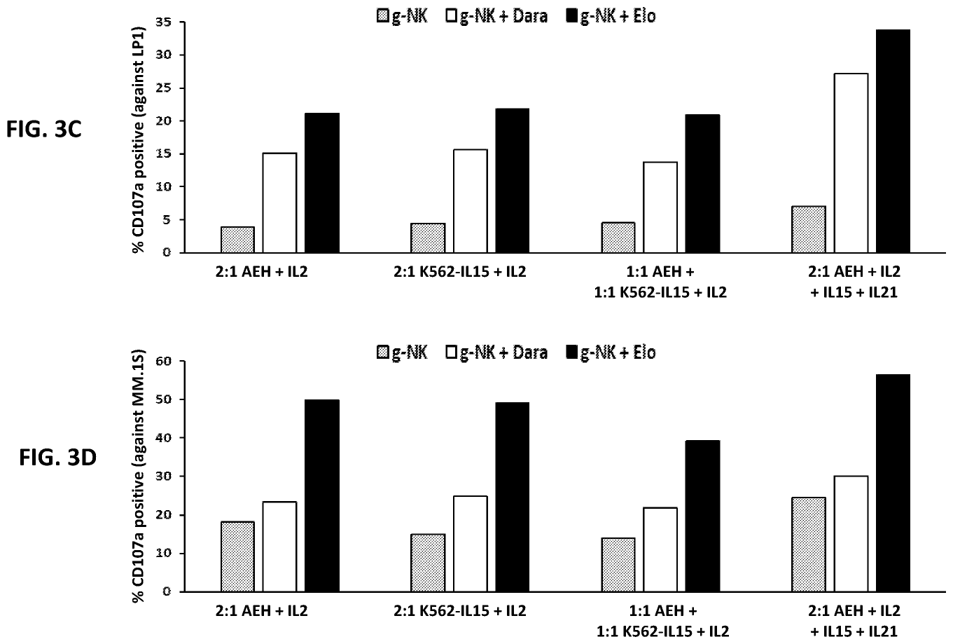

As shown in FIG. 4A-4D, after both 13 days (FIGS. 4A-4B) and 21 days (FIGS. 4C-4D) of expansion, g-NK cells expanded in the presence of IL-21 expressed more of the cytolytic protein perforin than did g-NK cells expanded without IL-21, as measured by both the percentage of perforin positive cells (FIG. 4A and FIG. 4C) and the total perforin expression (MFI) (FIG. 4B and FIG. 4D). In addition, after both 13 days and 21 days of expansion, g-NK cells expanded in the presence of IL-21 expressed more of the pro-apoptotic protein granzyme B than did g-NK cells expanded without IL-21, as measured by both the percentage of granzyme B positive cells (FIG. 4A and FIG. 4C) and the total granzyme B expression (MFI) (FIG. 4B and FIG. 4D).

Together, these results show that g-NK cells expanded in the presence of IL-21 have enhanced expression of perforin and granzyme B compared to g-NK cells expanded without IL-21.

### D. Interferon-γ Expression

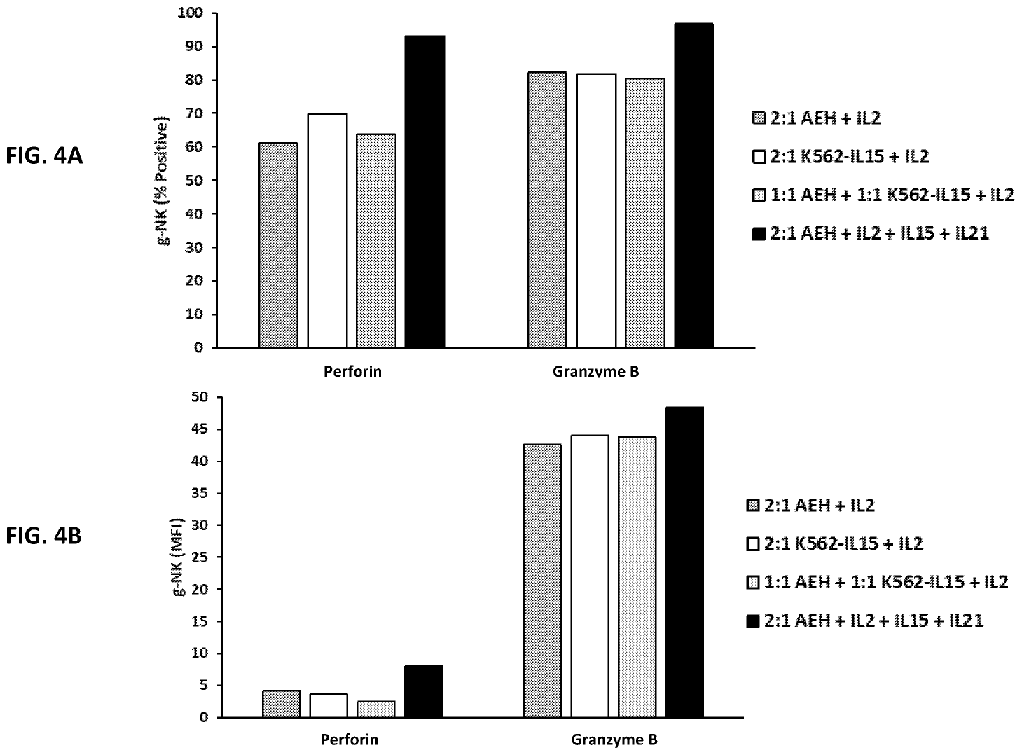

As shown in FIG. 5A-5D, after both 13 days (FIGS. 5A-5B) and 21 days (FIGS. 5C-5D) of expansion, g-NK cells expanded in the presence of IL-21 expressed more Interferon-γ against the CD38^{high} MM cell line LP1 (FIG. 5A and FIG. 5C) and the SLAMF7^{high} MM cell line MM.1S (FIG. 5B and FIG. 5D) than did g-NK cells expanded without IL-21. Greater Interferon-γ expression for IL-21 expanded g-NK cells was observed in the absence of antibody as well as in the presence of either daratumumab or elotuzumab.

Together, these results show that g-NK cells expanded in the presence of IL-21 have enhanced Interferon-γ expression against tumor cells compared to g-NK cells expanded without IL-21.

### E. TNF-α Expression

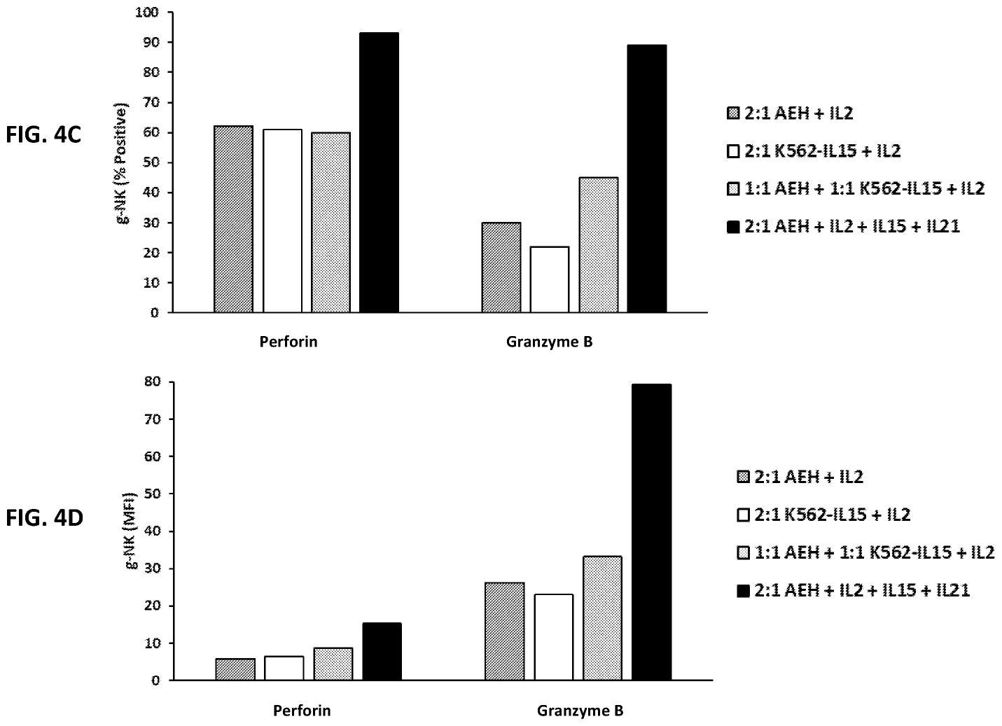

As shown in FIG. 6A-6D, after both 13 days (FIGS. 6A-6B) and 21 days (FIGS. 6C-6D) of expansion, g-NK cells expanded in the presence of IL-21 expressed more TNF-α against the CD38^{high} MM cell line LP1 (FIG. 6A and FIG. 6C) and the SLAMF7^{high} MM cell line MM.1S (FIG. 6B and FIG. 6D) than did g-NK cells expanded without IL-21. Greater TNF-α expression for IL-21 expanded g-NK cells was observed in the absence of antibody as well as in the presence of either daratumumab or elotuzumab.

Together, these results show that g-NK cells expanded in the presence of IL-21 have enhanced TNF-α expression against tumor cells compared to g-NK cells expanded without IL-21.

### Example 3: Expansion of g-NK Cells in the Presence of Additional Cytokines

In another study, the expansion rates of NK cells expanded in the presence of various combinations of cytokine mixtures and concentrations were compared. NK cells were harvested from the same donor as in Example 1 and as described above. NK cells were seeded at both a density and a subculture density of 2×10⁵ cells per mL, and they were co-cultured with irradiated 221.AEH feeder cells at a 2:1 221.AEH to NK cell ratio. For the NK cell expansions, cytokines were added at the following concentrations: IL-2 at 100 IU/mL (low IL-2) or 500 IU/mL (IL-2); IL-15 at 10 ng/mL; IL-21 at 25 ng/mL; IL-12 at 10 ng/mL; IL-18 at 10 ng/mL; and/or IL-27 at 10 ng/mL. All expansions were carried out in CellGenix GMP SCGM media supplemented with 5% human AB Serum and with the respective cytokines.

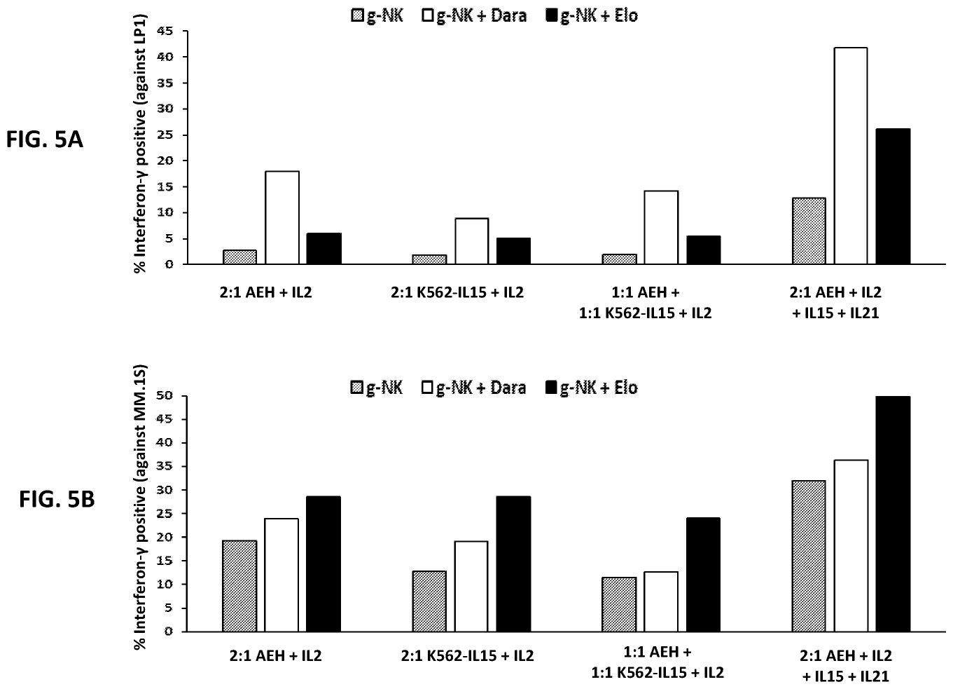

As shown in FIG. 7, NK cells expanded in the presence of IL-21 had a higher g-NK cell expansion rate than did NK cells expanded in the presence of IL-2 and IL-15; IL-12, IL-15, and IL-18; and IL-15, IL-18, and IL-27 by themselves. The combination of cytokines leading to the highest g-NK cell expansion rate was IL-2 and IL-21, either in the presence or absence of IL-15.

Together, these results show that the presence of IL-21 improves g-NK cell expansion rate more so than does other cytokine mixtures.

### Example 4: Cell Effector Function of g-NK Cells Expanded in the Presence of Additional Cytokines

NK cell effector function was measured in g-NK cells expanded for 15 days in the presence of cytokines, including in the presence of IL-21, as described in Example 3. Assays were performed as described in Example 2 using target cell lines LP1 and MM.1S at a 0.5:1 NK to MM cell ratio and with antibodies daratumumab and elotuzumab.

### A. Cell Mediated Cytoxicity

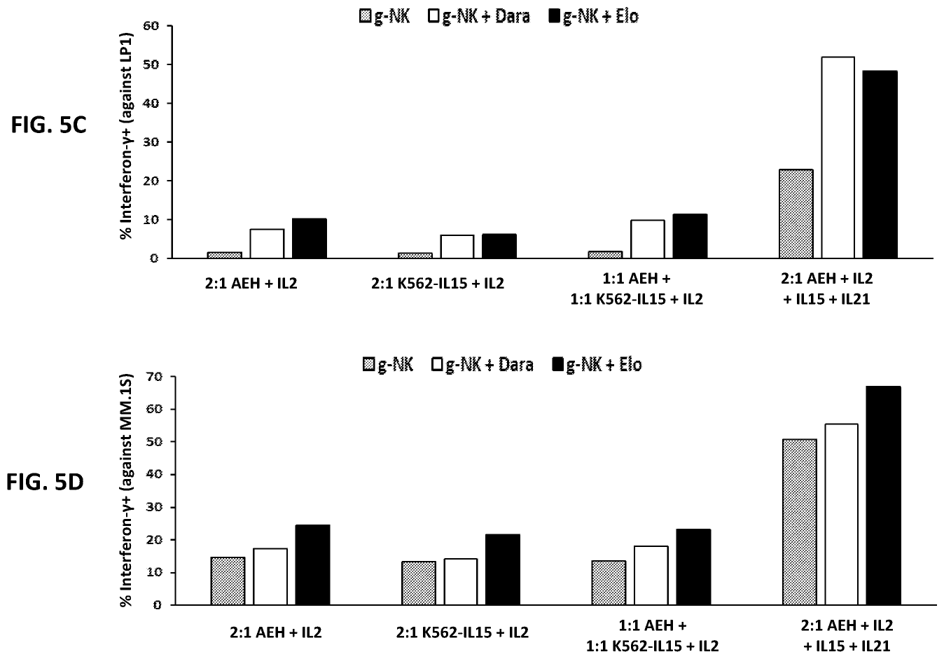

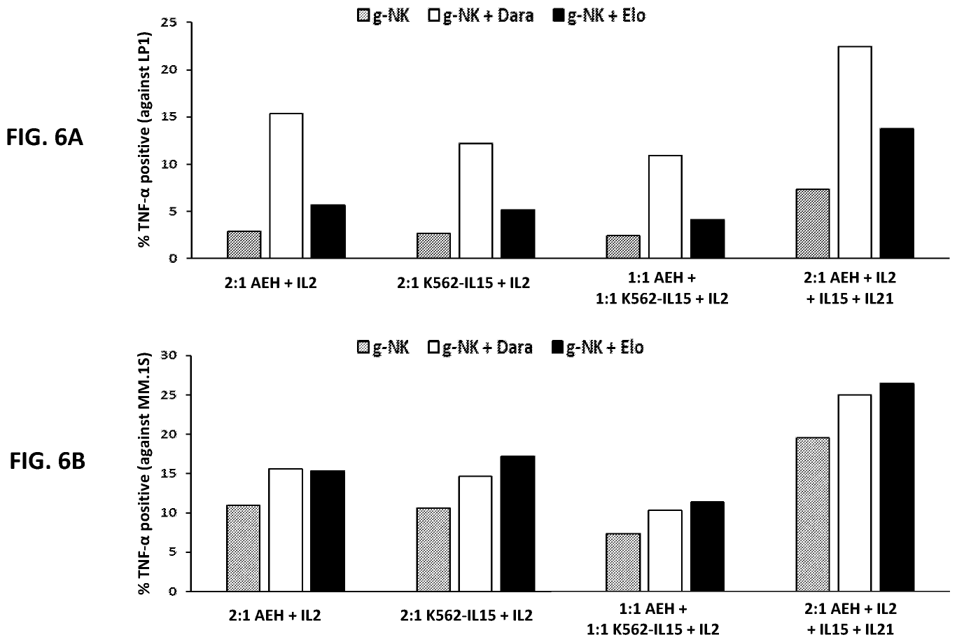

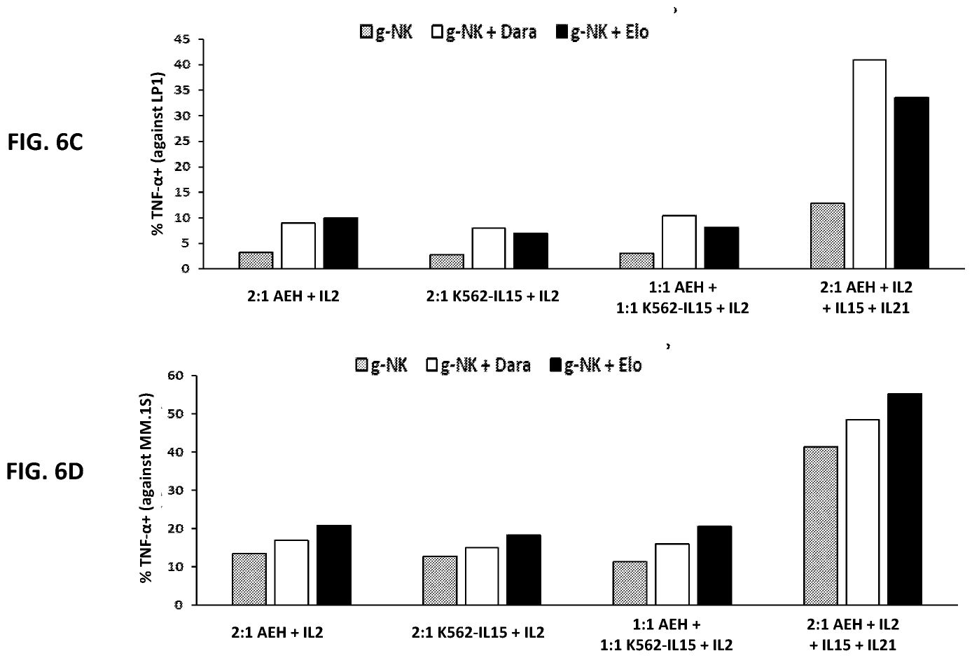

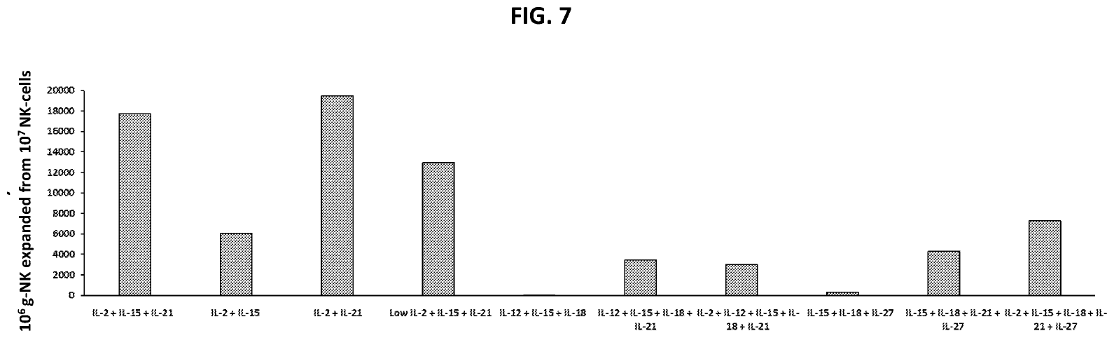

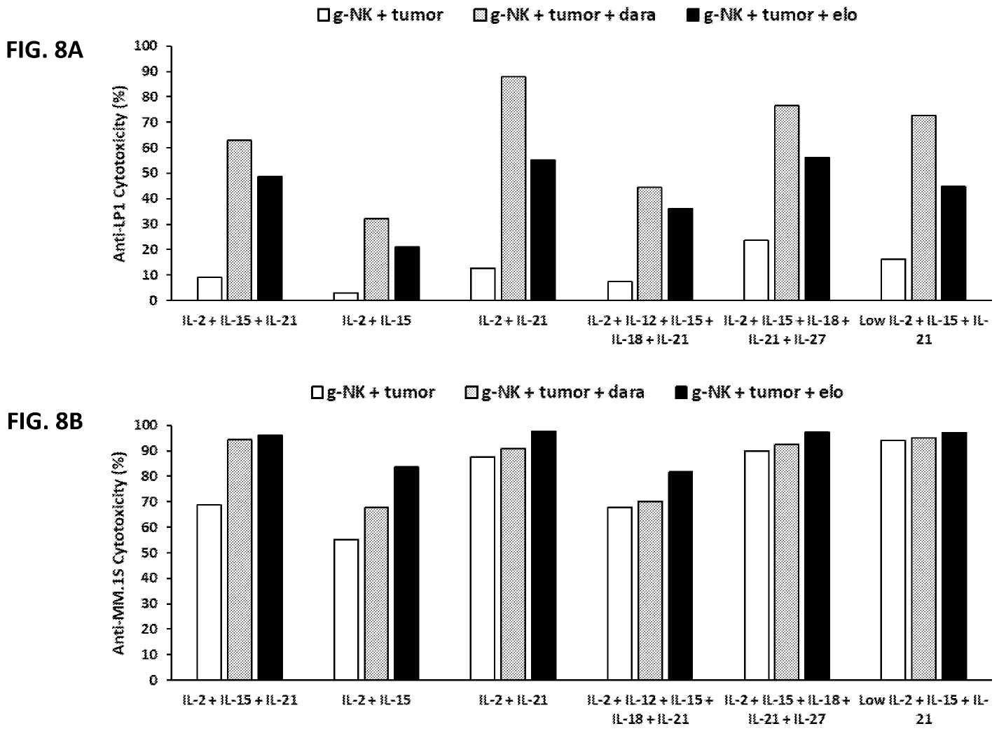

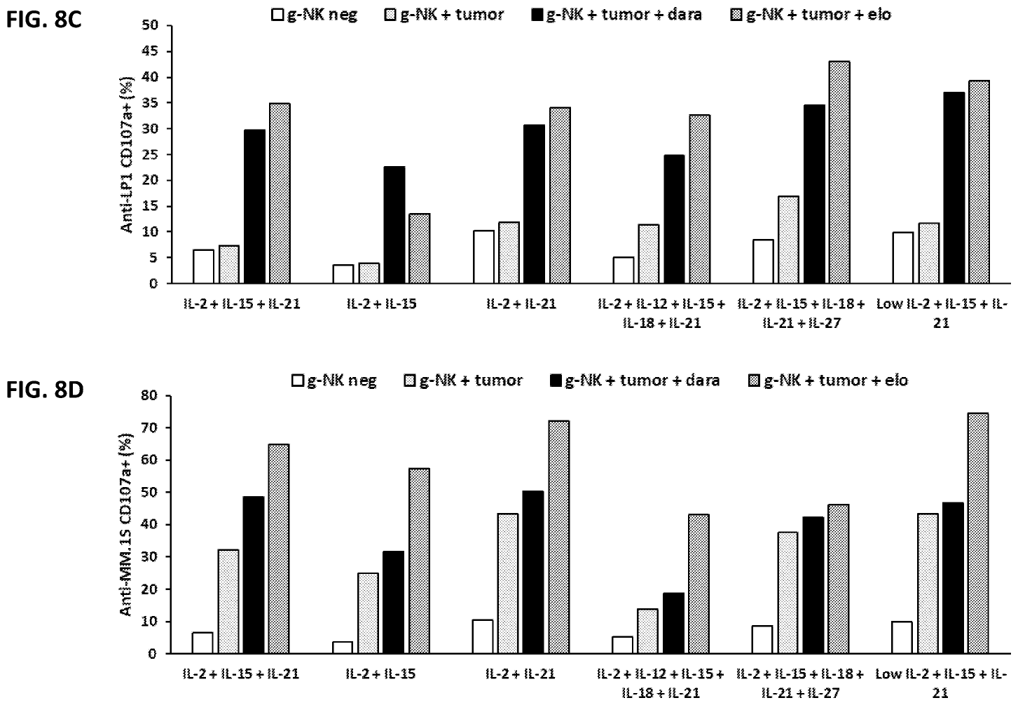

As shown in FIG. 8A and FIG. 8B, g-NK cells expanded in the presence of IL-2, IL-15, and IL-21 had greater cell-mediated cytotoxicity against the CD38^{high} MM cell line LP1 (FIG. 8A) and the SLAMF7^{high} MM cell line MM.1S (FIG. 8B) than did g-NK cells expanded in the presence of IL-2 and IL-15. Greater cell-mediated cytotoxicity for g-NK cells expanded in the presence of IL-2, IL-15, and IL-21 was observed in the absence of antibody as well as in the presence of either daratumumab or elotuzumab.

Together, these results show that g-NK cells expanded in the presence of IL-2, IL-15, and IL-21 have enhanced cell-mediated cytotoxicity against tumor cells compared to g-NK cells expanded in the presence of IL-2 and IL-15.

### B. Degranulation

As shown in FIG. 8C and FIG. 8D, g-NK cells expanded in the presence of IL-2, IL-15, and IL-21 degranulated more against the CD38^{high} MM cell line LP1 (FIG. 8C) and the SLAMF7s MM cell line MM.1S (FIG. 8D) than did g-NK cells expanded in the presence of IL-2 and IL-15. Greater degranulation for g-NK cells expanded in the presence of IL-2, IL-15, and IL-21 was observed under all conditions, including in the absence of antibody.

Together, these results show that g-NK cells expanded in the presence of IL-2, IL-15, and IL-21 have enhanced degranulation against tumor cells compared to g-NK cells expanded in the presence of IL-2 and IL-15.

### C. Perforin and Granzyme B Expression

As shown in FIG. 8E and FIG. 8F, g-NK cells expanded in the presence of IL-2, IL-15, and IL-21 expressed more of the cytolytic protein perforin than did g-NK cells expanded in the presence of IL-2 and IL-15, as measured by both the percentage of perforin positive cells (FIG. 8E) and the total perforin expression (MFI) (FIG. 8F). In addition, g-NK cells expanded in the presence of IL-2, IL-15, and IL-21 expressed more of the pro-apoptotic protein granzyme B than did g-NK cells expanded in the presence of IL-2 and IL-15, as measured by both the percentage of granzyme B positive cells (FIG. 8E) and the total granzyme B expression (MFI) (FIG. 8F). Addition of IL-2, IL-15, IL-18, IL-21, and IL-27 to expansion media enhanced granzyme B expression by g-NK cells.

Together, these results show that g-NK cells expanded in the presence of IL-2, IL-15, and IL-21 have enhanced expression of perforin and granzyme B compared to g-NK cells expanded in the presence of IL-2 and IL-15.

### D. Interferon-γ Expression

As shown in FIG. 8G-8H, g-NK cells expanded in the presence of IL-2, IL-15, and IL-21 expressed more Interferon-γ against the CD38^{high} MM cell line LP1 (FIG. 8G) and the SLAMF7^{high} MM cell line MM.1S (FIG. 8H) than did g-NK cells expanded in the presence of IL-2 and IL-15. Greater Interferon-γ expression for g-NK cells expanded in the presence of IL-2, IL-15, and IL-21 was observed under all conditions, including in the absence of antibody. Addition of IL-2, IL-12, IL-15, IL-18, and IL-21 to expansion media enhanced interferon-γ expression by g-NK cells under all conditions, including in the absence of antibody. Addition of IL-2, IL-15, IL-18, IL-21, and IL-27 to expansion media enhanced interferon-γ expression by g-NK cells under all conditions, including in the absence of antibody.

Together, these results show that g-NK cells expanded in the presence of IL-2, IL-15, and IL-21 have enhanced Interferon-γ expression against tumor cells compared to g-NK cells expanded in the presence of IL-2 and IL-15.

### E. TNF-α Expression

As shown in FIG. 8I-8J, g-NK cells expanded in the presence of IL-2, IL-15, and IL-21 expressed more TNF-α against the CD38^{high} MM cell line LP1 (FIG. 8I) and the SLAMF7^{high} MM cell line MM.1S (FIG. 8J) than did g-NK cells expanded in the presence of IL-2 and IL-15. Greater TNF-α expression for g-NK cells expanded in the presence of IL-2, IL-15, and IL-21 was observed under all conditions, including in the absence of antibody. Addition of IL-2, IL-15, IL-18, IL-21, and IL-27 to expansion media enhanced antibody-induced TNF-α expression by g-NK cells under all conditions, including in the absence of antibody.

Together, these results show that g-NK cells expanded in the presence of IL-2, IL-15, and IL-21 have enhanced TNF-α expression against tumor cells compared to g-NK cells expanded in the presence of IL-2 and IL-15.

### Example 5: Expansion and Cell Effector Function of g-NK Cells Expanded in the Presence of IL-21

In this study, the expansion rate and NK cell effector function of NK cells expanded in the presence of IL-21 were compared to that of NK cells expanded in the absence of IL-21. Human peripheral blood mononuclear cells (PBMC) were isolated by Histopaque® density centrifugation from whole blood from a CMV-positive human donor, or for comparison a CMV-seronegative donor, as per manufacturer's instructions. Donors were CMV-seropositive (n=8) and CMV seronegative (n=6) (Age 37.8±10.6 yrs; 8 males and 6 females).

PBMCs were harvested from buffy coat, washed, and assessed by flow cytometry for viable CD45^{pos} cells. NK cells were enriched by immunoaffinity-based magnetic bead separation using Miltenyi MACS™ Microbeads either by depletion of CD3^{pos} cells to remove T cells (CD3 depletion, CD3^{neg}) or by CD3 depletion followed by positive selection for CD57 to enrich CD57^{pos} NK cells (CD3^{neg}CD57^{pos}). The latter method of initially enriching for CD3^{neg}/CD57^{pos} cells prior to expansion was used in subsequent experiments for expanding g-NK cells. As a further comparison, NK cells were enriched by CD3 depletion followed by positive selection for CD16 (enrich CD16^{pos} NK cells and monocytes (CD3negCD57pos). NK cells were seeded at a density of 2×10⁵ cells per mL and a subculture density of 2×10⁵ cells per mL. The NK cells were co-cultured with gamma irradiated (100 Gy) 221.AEH feeder cells at a 2:1 221.AEH to NK cell ratio and expanded in the presence of IL-2 (500 IU/mL), IL-15 (10 ng/mL), and IL-21 (25 ng/mL); or IL-2 alone (500 IU/mL). A ratio of 1:1 irradiated 221.AEH feeder cells to NK cells was used if the PBMCs had been cryopreserved prior to enrichment of NK cells, as further described in Example 6. All expansions were carried out in CellGenix GMP SCGM media supplemented with 5% human AB Serum and with the respective cytokines. NK cells were expanded for 2 weeks and media was changed every 2-5 days. Expanded NK-cells were cryopreserved using 90% FBS and 10% DMSO for later use in functional assays.

Expansion and cell effector function were assessed after 14 days of expansion. Assays were performed as described in Example 2 using target cell lines LP1 and MM.1S at a 0.5:1 NK to MM cell ratio and with antibodies daratumumab and elotuzumab.

In some studies described in subsequent examples, phenotypic and functional activities of g-NK cells were compared to cNK cells. Due to insufficient yield of cNK cells from CMV-seronegative donors and preferential expansion of g-NK cells from CMV-seropositive donors using the above described method (results described in section A below), an alternative method was used to expand cNK cells for in vitro functional and in vivo studies. This expansion method used K652-mbIL15-41BBL feeder cells and 500 IU/mL IL-2 to expand cNK cells 180±89 fold (n=5 CMV^{neg}) over 2 weeks (Fujisaki et al., 2009 Cancer Res., 68(9):4010-4017). The proportion of g-NK cells in the 5 CMVneg donors (Age 38.9±9.8 yrs; 3 males and 2 females) was 1.5±0.5% before and 1.6±0.4% after expansion.

A. Expansion Rate of g-NK Cells

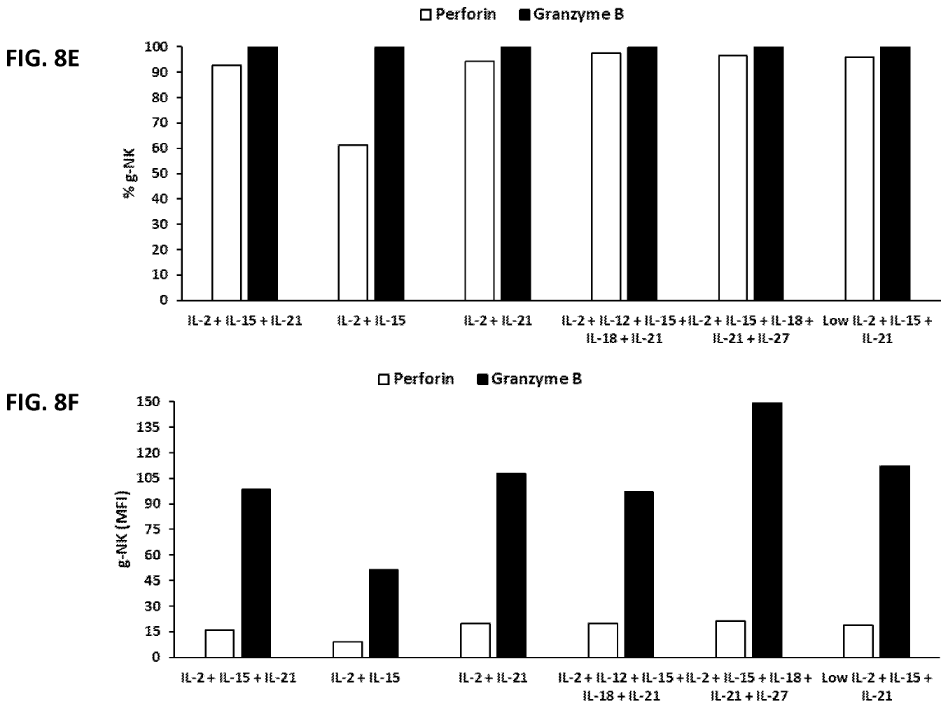

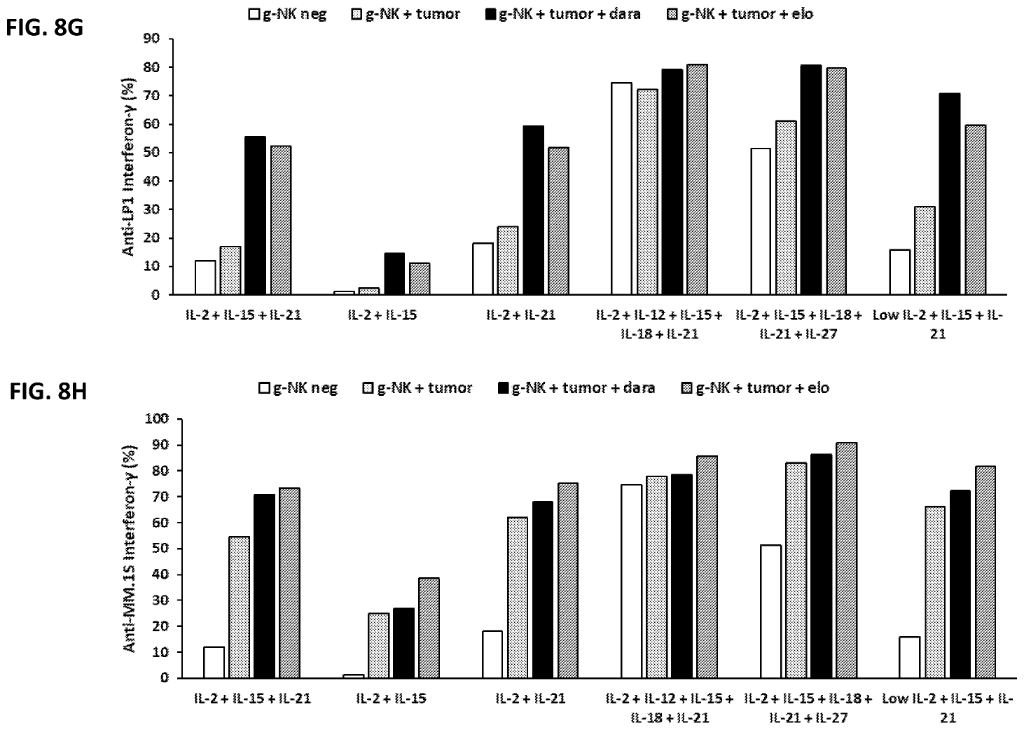

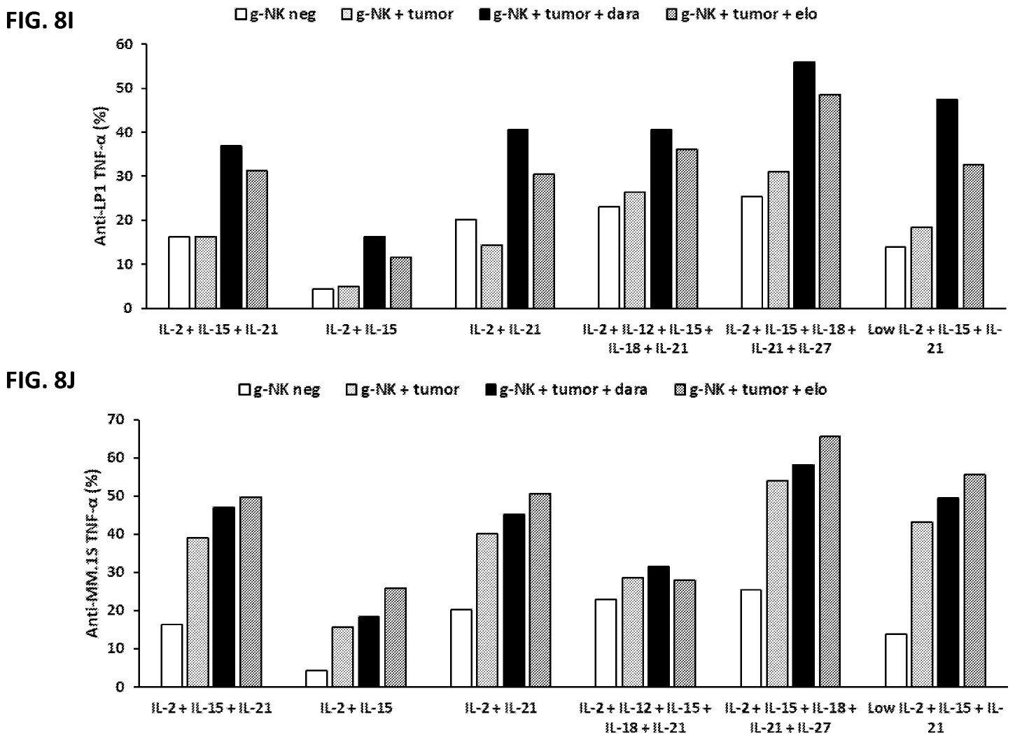

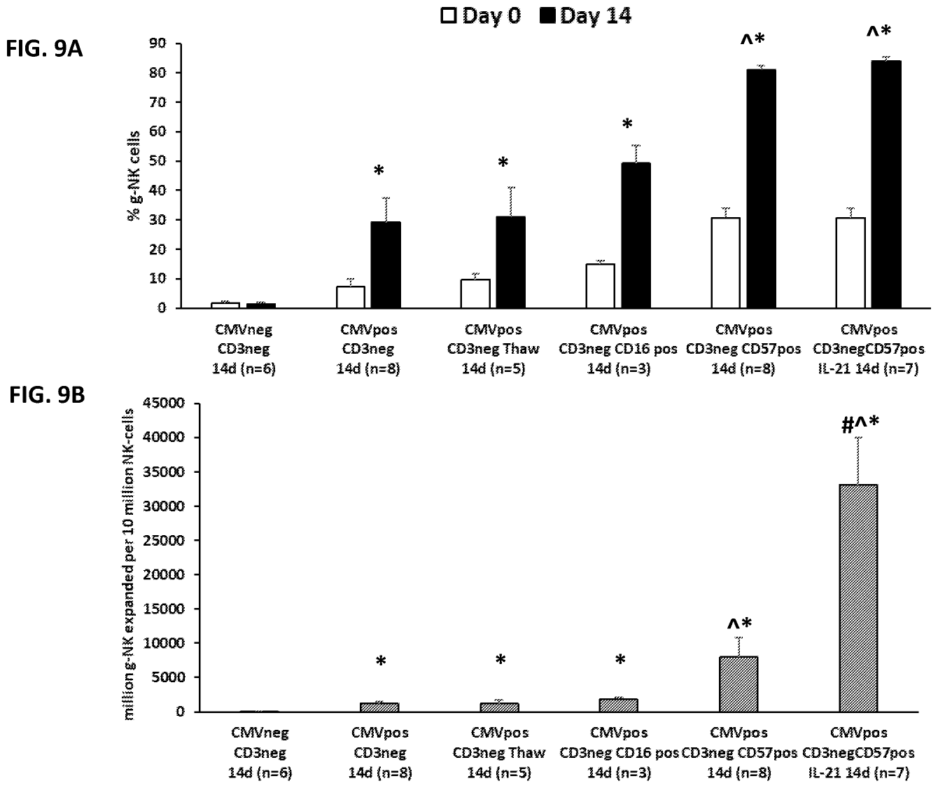

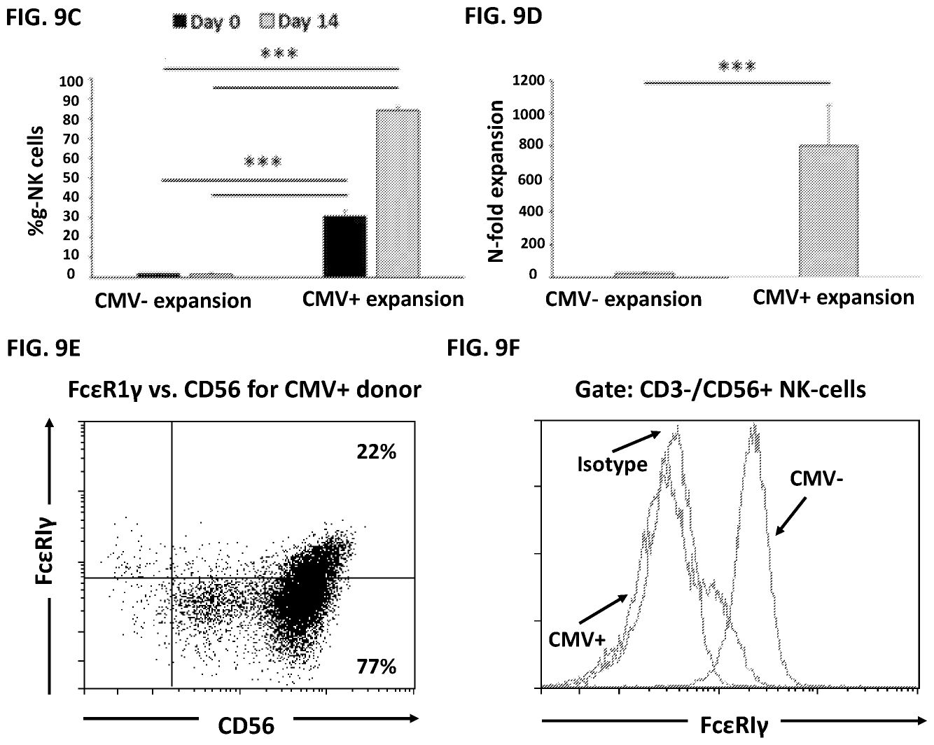

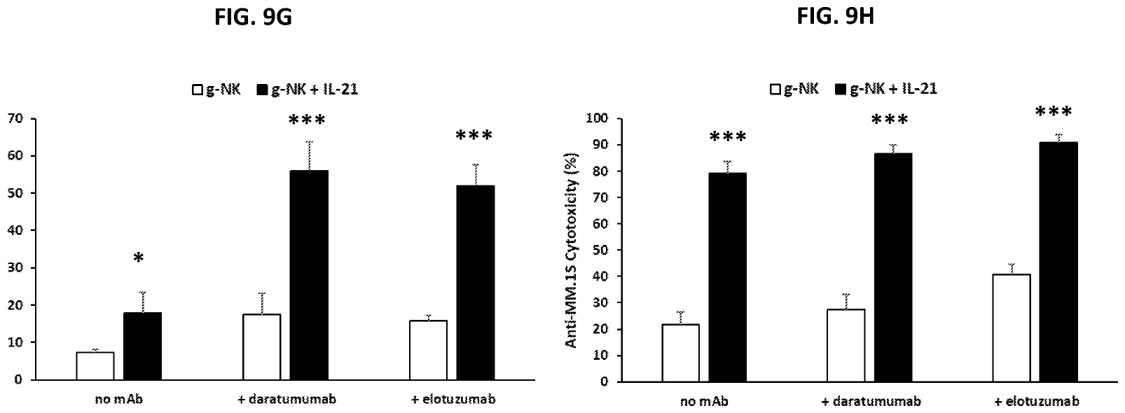

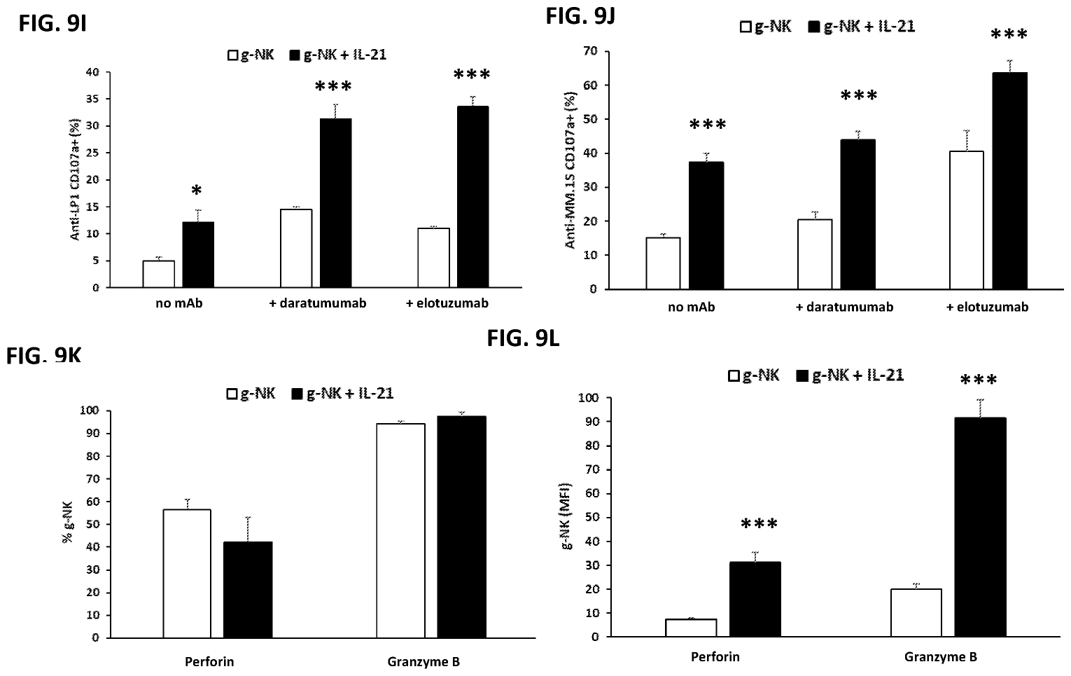

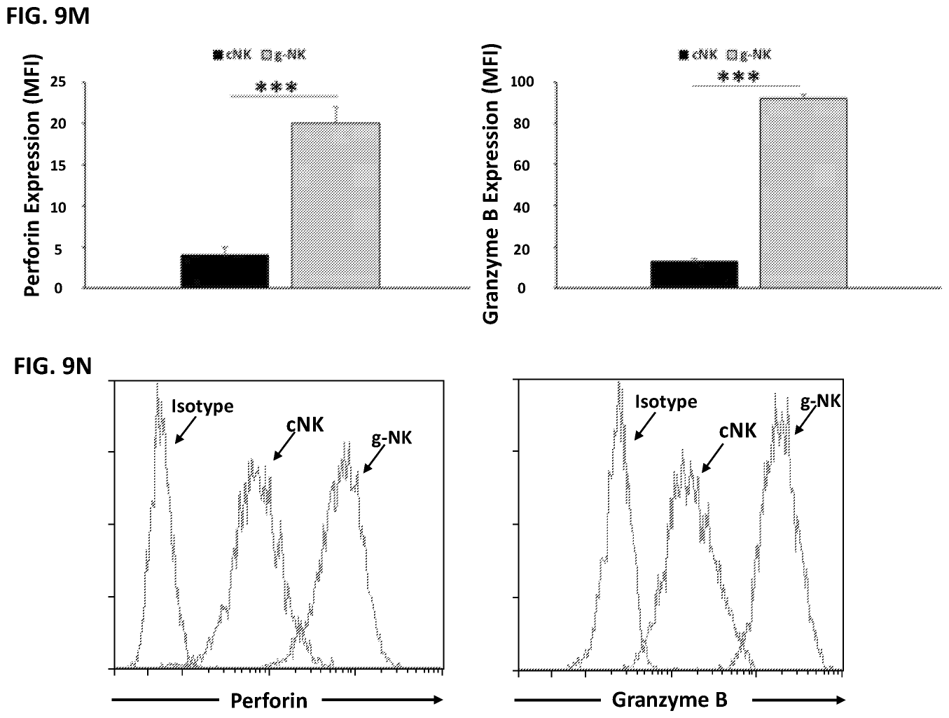

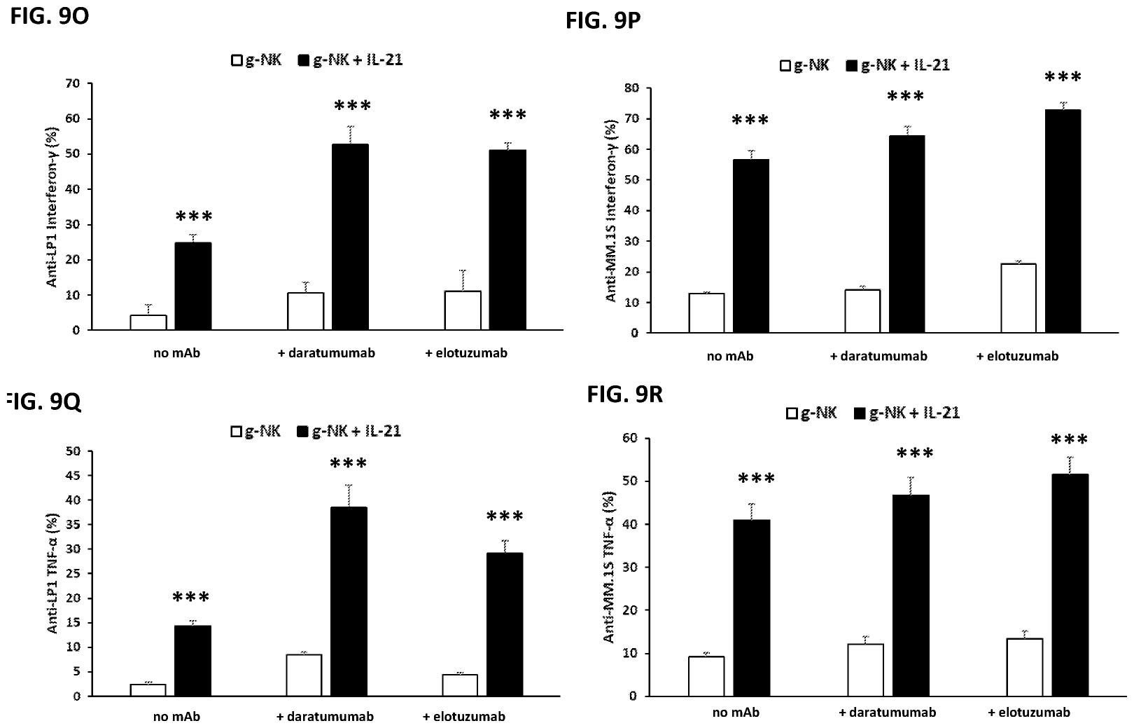

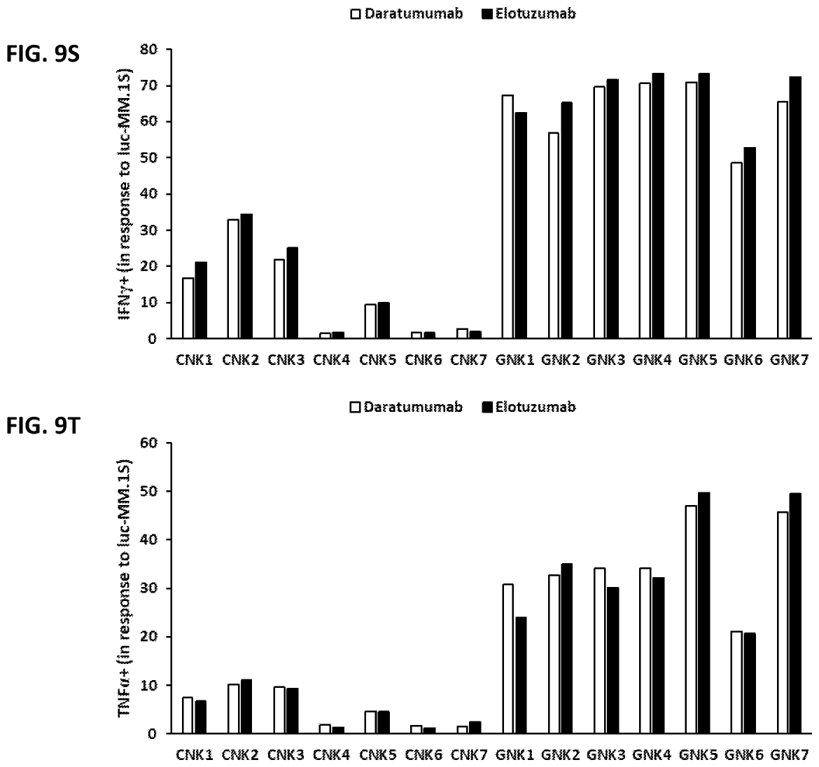

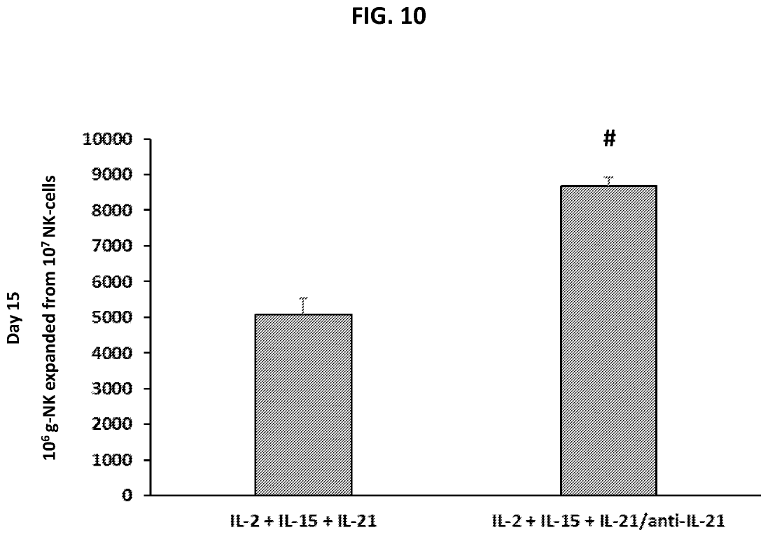

Cells were counted at media change and the percentage of g-NK cells was assessed by flow cytometry at day 0 and day 14. As shown in FIG. 9A and FIG. 9B, NK cells that has been initially enriched for CD3^{neg}/CD57^{pos} cells prior to expansion and then expanded in the presence of IL-21 had higher g-NK cell expansion rates than the similar conditions but without IL-21. As measured using intracellular staining of FcRγ and flow cytometry, higher g-NK cell expansion rates were observed when measuring both the percentage (FIG. 9A) and count (FIG. 9B) of g-NK cells.

Prior to expansion, the proportion of g-NK cells in the CMV seropositive donors was 30.8±3.1% (% of total NK-cells), while the proportion of g-NK cell was only 1.8±0.3% (% of total NK-cells) in the CMV seronegative donors. Following expansion after initial enrichment for CD3^{neg}/CD57^{pos} cells, the proportion of g-NK cells was increased to 84.0 1.4% for CMV-seropositive donors, but was unchanged for CMV-seronegative donors (1.5±0.4%) (FIG. 9C). Representative flow cytometry dot plots and histograms depicting the proportion of g-NK cells in CMV seropositive and seronegative donors are shown in FIGS. 9E and 9F. The percentage of NKG2Cpos/NKG2Aneg NK-cells within the g-NK subset ranged from 1.7 to 51% (26.8±13.9%). Thus, there is a phenotypic overlap between g-NK and NKG2C^{pos}/NKG2C^{neg} NK-cells but they are not identical.

A representative expansion of g-NK cells is shown in FIG. 9D, in which it is shown that the expansion method increased the proportion of g-NK cells from a CMV-seropositive donor with a detectable g-NK population with at least a 400-fold increase in overall NK-cell number.

Together, these results show that the presence of IL-21 improves g-NK cell expansion.

### B. Cell Mediated Cytoxicity

As shown in FIG. 9G and FIG. 9H, NK cells expanded in the presence of IL-21 had greater cell-mediated cytotoxicity against the CD38^{high} MM cell line LP1 (FIG. 9G) and the SLAMF7^{high} MM cell line MM.1S (FIG. 9H) than did g-NK cells expanded without IL-21. Greater cell-mediated cytotoxicity for IL-21 expanded g-NK cells was observed in the absence of antibody as well as in the presence of either daratumumab or elotuzumab.

Together, these results show that g-NK cells expanded in the presence of IL-21 have enhanced cell-mediated cytotoxicity against tumor cells compared to g-NK cells expanded without IL-21.

### C. Degranulation

As shown in FIG. 9I and FIG. 9J, g-NK cells expanded in the presence of IL-21 degranulated more against the CD38^{high} MM cell line LP1 (FIG. 9I) and the SLAMF7^{high} MM cell line MM.1S (FIG. 9J) than did g-NK cells expanded without IL-21. Greater degranulation for IL-21 expanded g-NK cells was observed in the absence of antibody as well as in the presence of either daratumumab or elotuzumab.

Together, these results show that g-NK cells expanded in the presence of IL-21 have enhanced degranulation against tumor cells compared to g-NK cells expanded without IL-21.

### D. Perforin and Granzyme B Expression

As shown in FIG. 9K and FIG. 9L, g-NK cells expanded in the presence of IL-21 expressed more of the cytolytic protein perforin than did g-NK cells expanded without IL-21, as measured by the total perforin expression (GMFI) (FIG. 9L), but not the percentage of perforin positive cells (FIG. 9K). In addition, g-NK cells expanded in the presence of IL-21 expressed more of the pro-apoptotic protein granzyme B than did g-NK cells expanded without IL-21, as measured by both the percentage of granzyme B positive cells (FIG. 9K) and the total granzyme B expression (GMFI) (FIG. 9L).

Baseline expression of perforin (FIG. 9M, left) and granzyme B (FIG. 9M, right) also was significantly higher in expanded g-NK cells than cNK cells (n=5). Representative histograms of perforin and granzyme B expression for NK and cNK cells is shown in FIG. 9N.

Together, these results show that g-NK cells expanded in the presence of IL-21 have enhanced expression of perforin and granzyme B against tumor cells compared to g-NK cells expanded without IL-21.

### E. Interferon-γ Expression

As shown in FIG. 9O and FIG. 9P, g-NK cells expanded in the presence of IL-21 expressed more Interferon-γ against the CD38^{high} MM cell line LP1 (FIG. 9O) and the SLAMF7^{high} MM cell line MM.1S (FIG. 9P) than did g-NK cells expanded without IL-21. Greater Interferon-γ expression for IL-21 expanded g-NK cells was observed in the absence of antibody as well as in the presence of either daratumumab or elotuzumab.

Together, these results show that g-NK cells expanded in the presence of IL-21 have enhanced Interferon-γ expression against tumor cells compared to g-NK cells expanded without IL-21.

### F. TNF-α Expression

As shown in FIG. 9Q and FIG. 9R, g-NK cells expanded in the presence of IL-21 expressed more TNF-α against the CD38^{high} MM cell line LP1 (FIG. 9Q) and the SLAMF7^{high} MM cell line MM.1S (FIG. 9R) than did g-NK cells expanded without IL-21. Greater TNF-α expression for IL-21 expanded g-NK cells was observed in the absence of antibody as well as in the presence of either daratumumab or elotuzumab.

Together, these results show that g-NK cells expanded in the presence of IL-21 have enhanced TNF-α expression against tumor cells compared to g-NK cells expanded without IL-21.

G. Comparison of Effector Functions Amongst g-NK Donors

g-NK cells and cNK cells were expanded as described and effector activity was compared amongst the different donors. Assays were performed as described in Example 2 using target cell line MM.1S at a 0.5:1 NK to MM cell ratio and with antibodies daratumumab and elotuzumab. After co-culture, the cells were fixed and permeabilized and analyzed by intracellular cytokine staining for Interferon-gamma (IFNγ) and TNF-alpha (TNFα). Results depicted in FIG. 9S (IFNγ) and FIG. 9T (TNFα) show that donor variability amongst g-NK donors is low, with a standard error of less than 5 for mAb-dependent IFNγ and TNFα response. Similar results were seen for other effector functions. The results showed that effector functions of all g-NK donors were superior to all cNK donors tested.

### Example 6: Expansion of g-NK Cells in the Presence of IL-21/Anti-IL-21 Complexes

Cryopreserved PBMCs were thawed and enriched for CD3^{neg}CD57^{pos} NK cells via magnetic sorting. Prior to expansion of these NK cells, IL-21/anti-IL-21 complexes were formed by combining IL-21 and an anti-IL-21 antibody. IL-21 and anti-IL-21 antibody were co-incubated for 30 minutes at 37° C. and at concentrations of 25 ng/mL and 250 ng/mL, respectively. The complexes, along with 500 IU/mL IL-2 and 10 ng/mL IL-15, were then added to the NK cell expansion media. NK cells were co-cultured with irradiated 221.AEH feeder cells at a 1:1 NK to 221.AEH feeder cell ratio. For comparison, NK cells were also expanded in the presence of IL-2, IL-15, and IL-21 at concentrations of 500 IU/mL, 10 ng/mL, and 25 ng/mL, respectively.

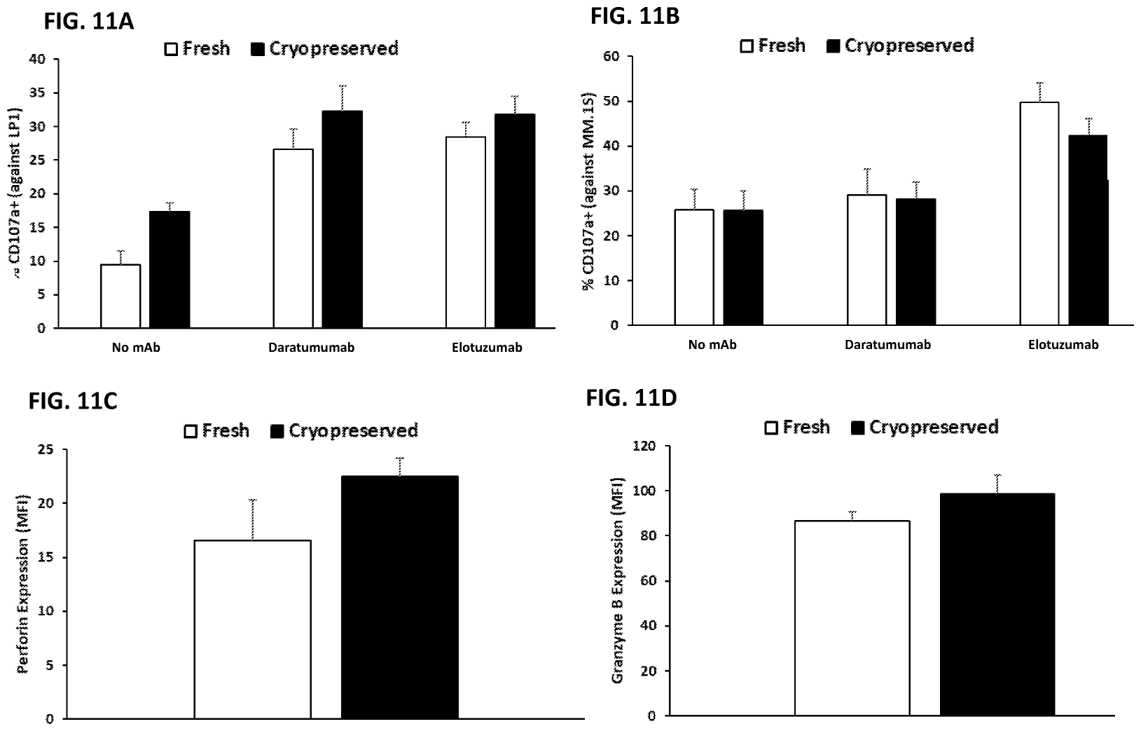

As shown in FIG. 10, g-NK cells expanded in the presence of IL-2, IL-15, and the IL-21/anti-IL-21 complex had a higher expansion rate than did g-NK cells expanded in the presence of IL-2, IL-15, and IL-21.

### Example 7: Maintenance of g-NK Cell Effector Function after Cryopreservation

NK cell effector function of previously cryopreserved g-NK cells was compared to that of freshly enriched (i.e., non-cryopreserved) g-NK cells (n=4). CD3^{neg}/CD57^{pos} enriched NK cells were co-cultured with irradiated 221.AEH feeder cells at a 2:1 221.AEH to NK cell ratio and in the presence of 500 IU/mL of IL-2, 10 ng/mL of IL-15, and 25 ng/mL of IL-21. After expansion, NK cells were functionally assessed fresh or were cryopreserved in 90% FBS with 10% DMSO and at a concentration of 20 million cells per 1.8 ml of cryopreservation media. NK cell effector functions against LP1 and MM.1S cell lines were assessed without antibody as well as in the presence of one μg/mL daratumumab or one μg/mL elotuzumab.

### A. Degranulation

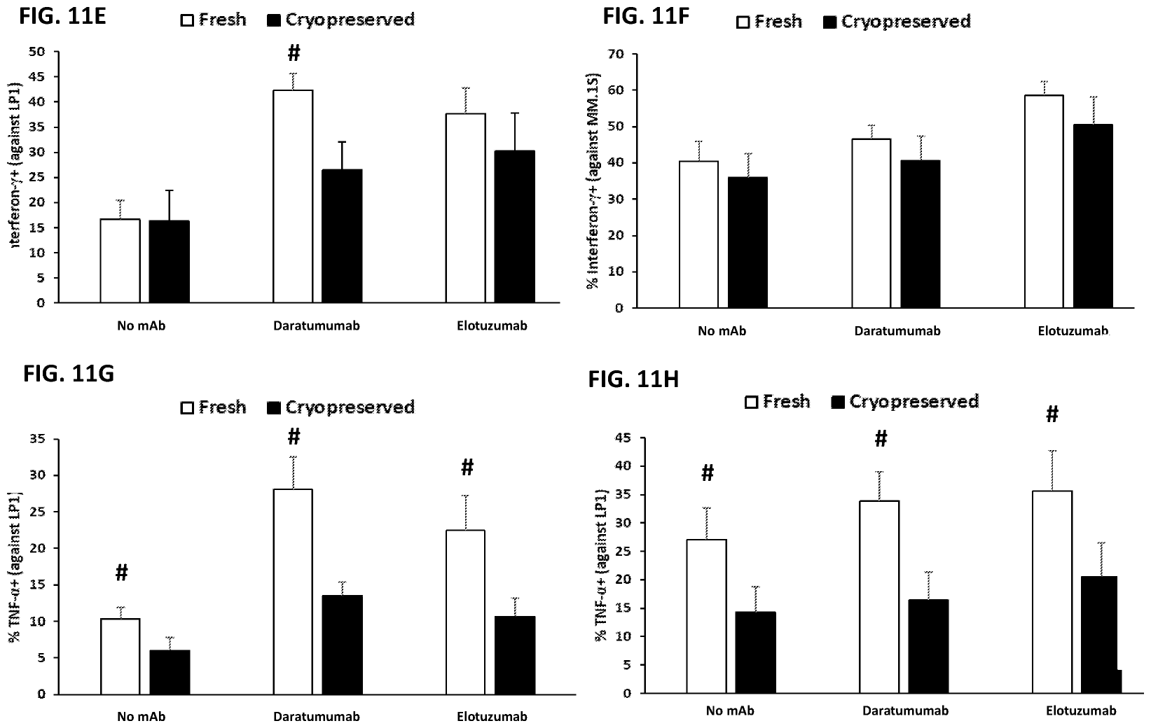

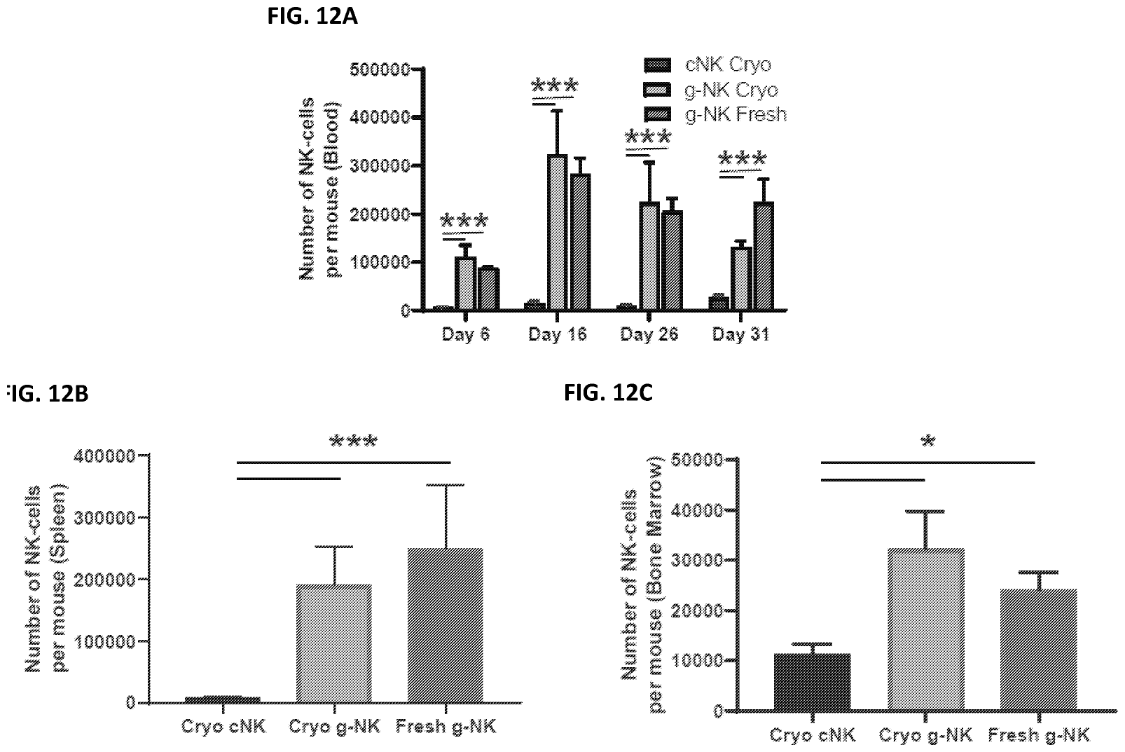

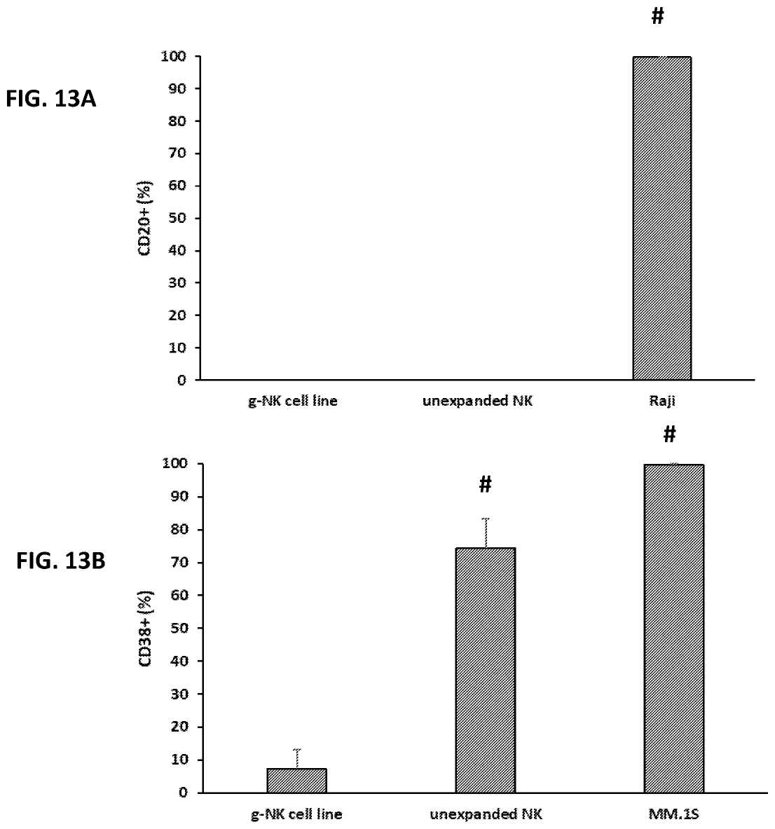

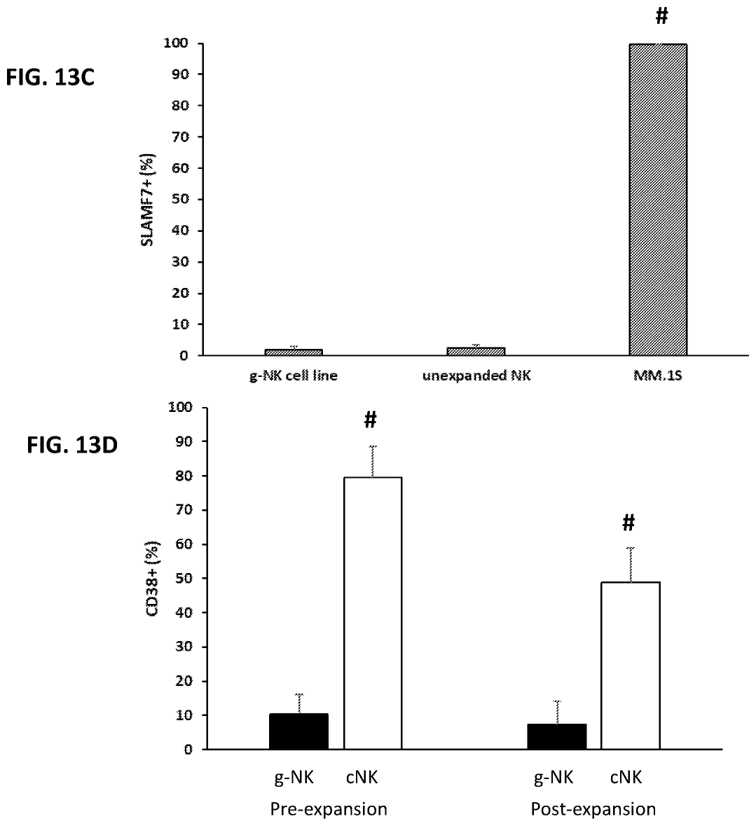

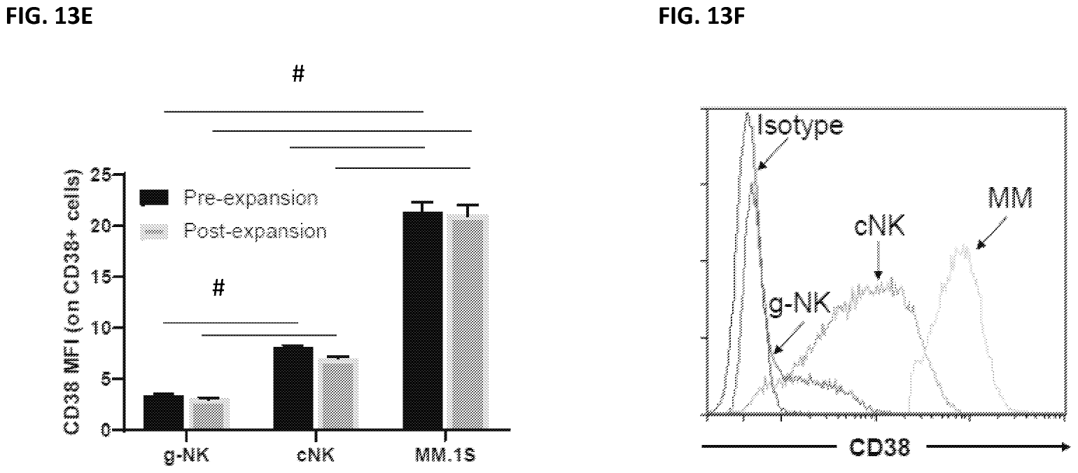

As shown in FIG. 11A and FIG. 11B, previously cryopreserved g-NK cells had degranulation levels comparable to that of fresh g-NK cells against the CD38^{high} MM cell line LP1 (FIG. 11A) and the SLAMF7^{high} MM cell line MM.1S (FIG. 11B). Comparable degranulation levels were observed in the absence of antibody as well as in the presence of either daratumumab or elotuzumab.

Together, these results show that g-NK cell degranulation in response to multiple myeloma target cells is maintained after cryopreservation.

### B. Perforin and Granzyme B Expression

As shown in FIG. 11C and FIG. 11D, previously cryopreserved g-NK cells had perforin (FIG. 11C) and granzyme B expression (FIG. 11D) comparable to that of fresh g-NK cells. Together, these results show that g-NK cell perforin and granzyme B expression is maintained after cryopreservation.

### C. Interferon-γ Expression

As shown in FIG. 11E and FIG. 11F, previously cryopreserved g-NK cells had Interferon-γ expression levels comparable to that of fresh g-NK cells against the CD38^{high} MM cell line LP1 (FIG. 11E) and the SLAMF7^{high} MM cell line MM.1S (FIG. 11F). Comparable Interferon-γ expression was observed in the absence of antibody as well as in the presence of either daratumumab or elotuzumab.

Together, these results show that g-NK cell Interferon-γ expression in response to multiple myeloma target cells is maintained after cryopreservation.

### D. TNF-α Expression

As shown in FIG. 11G and FIG. 11H, previously cryopreserved g-NK cells had decreased TNF-α expression levels compared to that of fresh g-NK cells against the CD38^{high} MM cell line LP1 (FIG. 11G) and the SLAMF7^{high} MM cell line MM.1S (FIG. 11H). Decreased TNF-α expression was observed in the absence of antibody as well as in the presence of either daratumumab or elotuzumab.

Together, these results show that g-NK cell TNF-α expression in response to multiple myeloma target cells is decreased after cryopreservation.

### Example 8: Assessment of Persistence of g-NK Cells In Vivo Compared to cNK Cells

NK cells, expanded substantially as described in Example 5, were injected into mice and biological samples were subjected to analysis using flow cytometry to assess their persistence.

As described in Example 5, g-NK cells were expanded after initially enriching for CD3^{neg}/CD57^{pos} cells from cryopreserved PBMCs, followed by expansion with irradiated 221.AEH feeder cells at a 1:1 221.AEH to NK cell ratio and in the presence of IL-2 (500 IU/mL), IL-15 (10 ng/mL), and IL-21 (25 ng/mL) stimulatory cytokines. The alternative method described in Example 5 was used to expand cNK cells due to insufficient yield of cNK cells from CMV-seronegative donors. cNK cells were expanded for 2 weeks using the transgenic leukemia cell line K562-mb15-41BBL and IL-2. All cells were expanded from cryopreserved PBMCs and cryopreserved feeder cells. Freeze media for the cryopreserved cells was CS-10 (Biolife Solutions, Bothel, WA, USA). Cryopreserved cell products were thawed rapidly in a hot water bath prior to being administered to the mice (37° C.).

A single dose of 1×10⁷ expanded NK cells (fresh g-NK, cryopreserved g-NK, or cryopreserved cNK cells) were intravenously injected via the tail vein into female NOD.Cg-PrkDc^{scid} IL2rg^{tm1wjl}/SzJ (NSG) mice (n=9, 3 per group). To provide NK-cell support, about 2 μg/mouse human recombinant IL-15 was administered via the I.P. route every three days (see Table 2). Blood collected at days 6, 16, 26, and 31 days post-infusion was immediately analyzed by flow cytometry. Mice were sacrificed at day 31, and bone marrow and spleen were harvested for immediate flow cytometry analysis.

TABLE 2
Persistence Study Design

| Group | | Number | Day of | Days of blood |
|---|---|---|---|---|
| Number | Arm | of Mice | NK dose | collection |
| 1 | IL-15 + Fresh day | 3 | 1 | 6, 16, 26, 31 (sac) |
| | 14 g-NK | | | |
| 2 | IL-15 + Cryo | 3 | 1 | 6, 16, 26, 31 (sac) |
| | day 14 g-NK | | | |
| 3 | IL-15 + Cryo | 3 | 1 | 6, 16, 26, 31 (sac) |
| | day 14 cNK | | | |

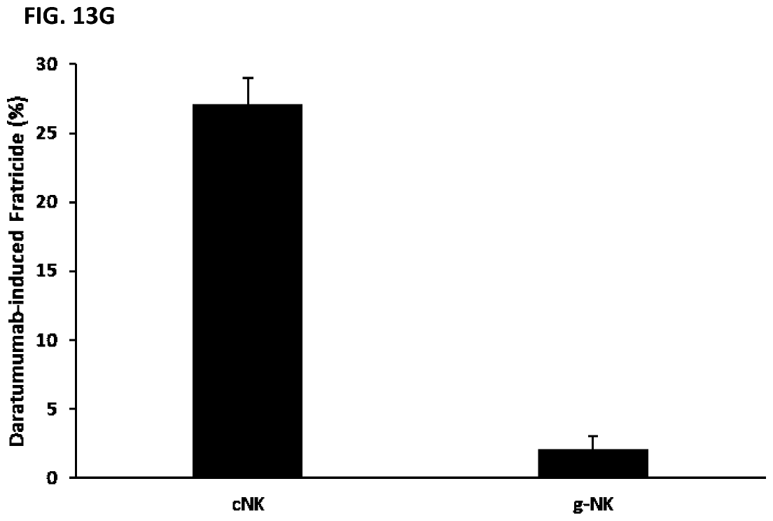

FIG. 12A-C shows enhanced persistence of fresh and cryopreserved g-NK cells relative to cNK cells in peripheral blood (FIG. 12A), spleen (FIG. 12B), and bone marrow (FIG. 12C). Persistence of cryopreserved g-NK cells was >90% greater than that seen with cryopreserved cNK cells in peripheral blood at multiple time points (p<0.001) (FIG. 12A), as well as spleen (p<0.001) (FIG. 12B) and bone marrow (p<0.05) (FIG. 12C) at sacrifice at day 31 (p<0.001). FIG. 12A also shows that levels of fresh and cryopreserved g-NK cells persisted at comparable levels until at least day 26 of the study.

The results are consistent with an observation that g-NK cells exhibit significantly improved persistence. These results demonstrate the utility of fresh or cryopreserved g-NK as a viable, off-the-shelf cellular therapy to enhance mAb ADCC.

### Example 9: Assessment of CD38 and SLAMF7 on g-NK Cells and Fratricide Activity of g-NK Cells

This example demonstrates, in part, the protection of g-NK cells from antibody due to lack of target surface markers.

g-NK cells were expanded substantially by the methods described in Example 5 with certain exceptions: 1) The ratio of 22.AEH target cells to NK cells was 2.5:1 (compared to a 2:1 ratio in Example 5), 2) NK cells were exposed to a lower level of IL-2 (100 IU/ml compared to 500 IU/ml in Example 5) and 3) IL-21 was absent during expansion. Approximately 2.0×10⁵ NK-cells and/or MM.1S or Raji cells were aliquoted into flow tubes and stained with 2 μL of 7-AAD viability dye and 2 μL of anti-CD45, 2 μL of anti-CD20, 2 μL of anti-CD38, 2 μL of anti-CD3, 10 μL of anti-SLAMF7, and 2 μL of anti-CD56 antibodies as described in Table E3. After a 10-minute incubation at 4° C., the cells were washed and intracellular staining was performed using an anti-FceRI antibody (Millipore). After completion of the staining process, the percentages of CD2, CD38, and SLAMF7 expressing g-NK, cNK, and MM.1S or Raji cells were assessed by 8-color flow cytometry (Miltenyi MACSQuant Analyzer 10).

TABLE E3
Flow cytometry panel to determine CD20, CD38, and
SLAMF7 expression on NK, MM, and Raji cells.

| Condition | V1 | V2 | B1 | B2 | B3 | B4 | R1 | R2 |
|---|---|---|---|---|---|---|---|---|
| NK Only | CD45 | CD20 | *FcRg | CD38 | 7-AAD | CD3 | SLAMF7 | CD56 |
| NK Only | CD45 | | *FcRg | CD38 | 7-AAD | CD3 | SLAMF7 | CD56 |
| CD20 FMO | | | | | | | | |
| NK Only | CD45 | CD20 | *FcRg | | 7-AAD | CD3 | SLAMF7 | CD56 |
| CD38 FMO | | | | | | | | |
| NK Only | CD45 | CD20 | *FcRg | CD38 | 7-AAD | CD3 | | CD56 |
| SLAMF7 FMO | | | | | | | | |
| NK + MM | CD45 | CD20 | *FcRg | CD38 | 7-AAD | CD3 | SLAMF7 | CD56 |
| NK + MM | CD45 | CD20 | *FcRg | | 7-AAD | CD3 | SLAMF7 | CD56 |
| CD38 FMO | | | | | | | | |
| NK + MM | CD45 | CD20 | *FcRg | CD38 | 7-AAD | CD3 | | CD56 |
| SLAMF7 FMO | | | | | | | | |
| MM Only | CD45 | CD20 | | CD38 | 7-AAD | | SLAMF7 | |
| MM Only | CD45 | CD20 | | | 7-AAD | | SLAMF7 | |
| CD38 FMO | | | | | | | | |
| MM Only | CD45 | CD20 | | CD38 | 7-AAD | | | |
| SLAMF7 FMO | | | | | | | | |
| NK + Raji | CD45 | CD20 | *FcRg | CD38 | 7-AAD | CD3 | SLAMF7 | CD56 |
| NK + Raji | CD45 | | *FcRg | CD38 | 7-AAD | CD3 | SLAMF7 | CD56 |
| CD20 FMO | | | | | | | | |
| Raji Only | CD45 | CD20 | | | 7-AAD | | | |
| Raji Only | CD45 | | | | 7-AAD | | | |
| CD20 FMO | | | | | | | | |

*FcRg is an intracellular epitope

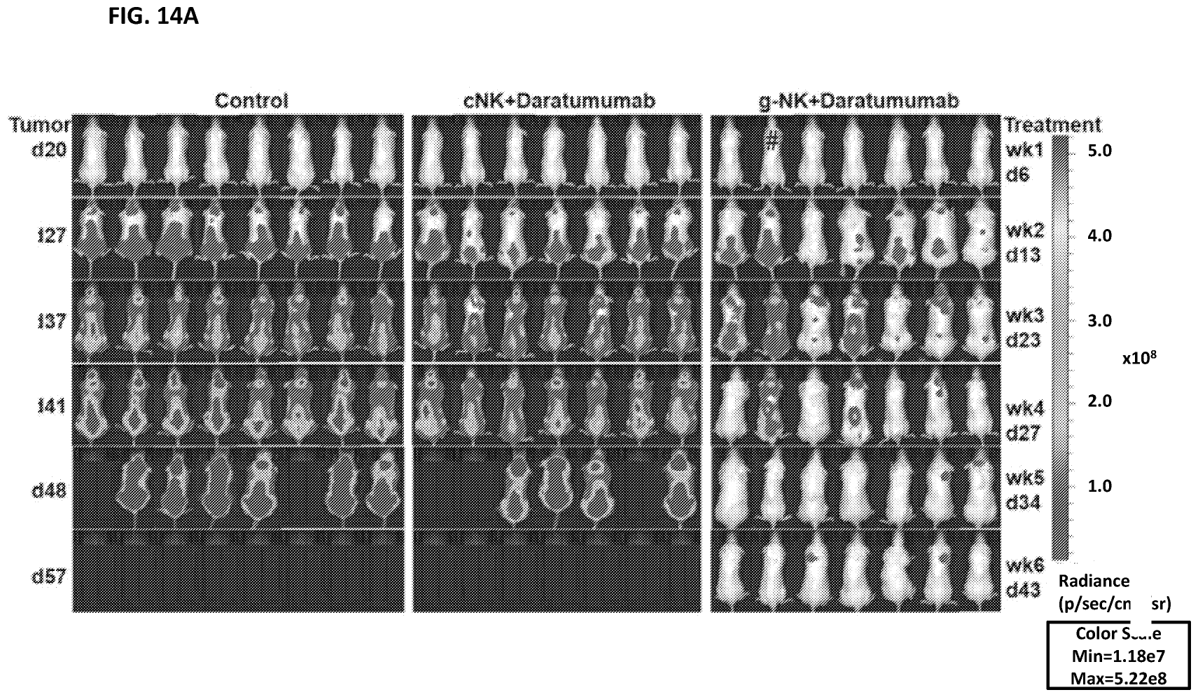

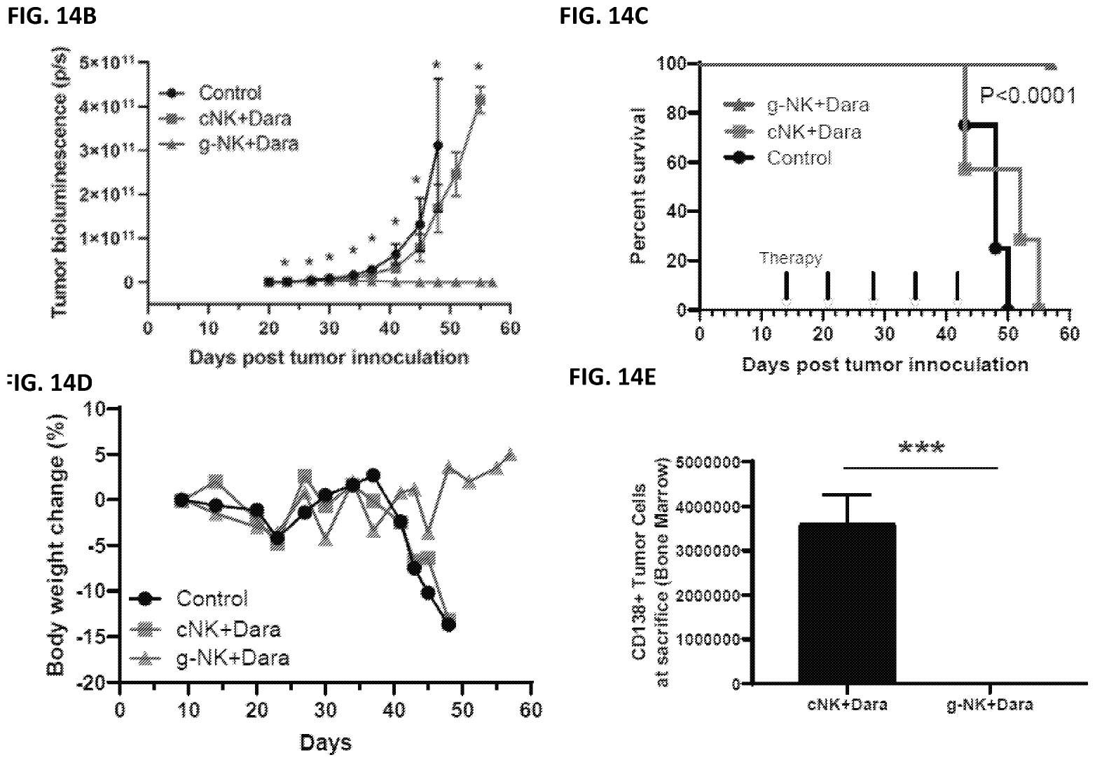

Expression of CD20, CD38, and SLAMF7 on g-NK, cNK, and MM.1S cells is presented in FIG. 13A-13D. Both g-NK and cNK lacked expression of CD20, which was highly expressed on Raji lymphoma cells (FIG. 13A). The expression of CD38 by g-NK was far less than for cNK and MM.1S cells (see FIG. 13B; p<0.001 for both). Expression of SLAMF7 was not different between g-NK and cNK (p=0.9), but both g-NK and cNK exhibited far lower expression of SLAMF7 than MM.1S cells (see FIG. 13C; p<0.001 for both). Reduced percentage of CD38^{pos} NK-cells was also seen on expanded g-NK when compared to expanded cNK (see FIG. 13D, p<0.001). Furthermore, intensity of CD38 expression (MFI) was reduced on CD38pos g-NK cells relative to CD38pos cNK and MM1/S cells (FIG. 13E, p<0.001). A representative histogram depicting the reduced CD38 expression of g-NK cells relative to cNK and MM.1S cells is shown in FIG. 13F.

The lack of CD20, CD38, or SLAMF7 expression by g-NK afforded protection from mAb-induced fratricide by rituximab (anti-CD20), daratumumab (anti-CD38), or elotuzumab (anti-SLAMF7). Overall, this data further illustrates how g-NK have a persistence advantage when compared to cNK, especially when in the presence of therapeutic antibodies such as daratumumab.

Similar results were observed by the expansion method described in Example 5 in the presence of IL-21, indicating that there is no difference in CD38 or SLAMF7 expression between g-NK cells expanded with or without IL-21. In a further assessment, the fratricide rate of expanded g-NK cells was compared to that of expanded cNK cells. As shown in FIGS. 13B and 13D-13F, CD38 expression was markedly lower on g-NK cells than cNK cells, and as shown in FIG. 13C equally low levels of SLAMF7 was present on g-NK and cNK cells. These results indicate the potential for lack of a fratricide effect by g-NK cells against these targets, since if NK cells express a mAb target an ADCC activity may lead to elimination of NK cells by fratricide in addition to the tumor. The finding that cNK cells express high levels of CD38 is consistent with prior results suggesting that >90% of CD38^{high} NK cells are depleted rapidly after daratumumab treatment in patients (Casneuf et al., 2017 Blood Adv, 1(23):2105-2114).

Six (6) unique donors were used to generate the expanded g-NK (6 CMV+, 3 M, 3F, age 39±7 years) and 8 unique donors were used to expand cNK (8 CMV−, 4 M, 4 F, age 38±9 years) using the methods substantially as described in Example 5. The proportion of g-NK was 85±4% for the g-NK donors and 2±1% for the cNK donors.

To assess fratricide, about 1×10⁴ expanded NK cells (g-NK or cNK) were cultured in the presence of 1 μg/mL daratumumab (anti-CD38). After a four-hour incubation at 37° C. in a 5% CO² incubator, the cells were washed and stained with anti-CD3 and anti-CD56 antibodies to quantify the number of NK cells. After a final wash, propidium iodide (PI) was added, and the number of live and dead NK-cells were resolved using 3-color flow cytometry (Bigley et al. (2016), Clin. Exp. Immunol., 185:239-251). As shown in FIG. 13G, g-NK cells have 13 times lower fratricide than cNK. Similar experiments carried out with elotuzumab showed that fratricide was not detected for g-NK or cNK treated with elotuzumab.

Together with the results of g-NK cells expanded in the absence of IL-21, these results are consistent with the ability of g-NK cells to confer enhanced mAb anti-tumor activity in MM without suffering from fratricide-related depletion.

### Example 10: In Vivo Efficacy in a Disseminated Orthotopic Xenograft MM.1S Model of Multiple Myeloma

The in vivo efficacy of NK cells (expanded g-NK cells or cNK cells) in combination with daratumumab was evaluated by measuring tumor inhibition and survival in a murine model of multiple myeloma. g-NK cells were expanded as described in Example 5 after initially enriching for CD3^{neg}/CD57^{pos} cells from cryopreserved PBMCs, followed by expansion with irradiated 221.AEH feeder cells at a 1:1 221.AEH to NK cell ratio and in the presence of IL-2 (500 IU/mL), IL-15 (10 ng/mL), and IL-21 (25 ng/mL) stimulatory cytokines. The alternative method described in Example 5 was used to expand cNK cells due to insufficient yield of cNK cells from CMV-seronegative donors. cNK cells were expanded for 2 weeks using the transgenic leukemia cell line K562-mb15-41BBL and IL-2. All cells were expanded from cryopreserved PBMCs and cryopreserved feeder cells.

Approximately 5×10⁵ luciferase-labeled MM.1S human myeloma cells were injected intravenously into to tail veins of female NSG mice and allowed to grow for 14 days. The monoclonal antibody daratumumab was administered via the I.P. route in combination with intravenous administration of 6.0×10⁶ expanded g-NK or cNK cells weekly, for a duration of five weeks. Beginning two weeks after tumor administration, 2 μg/mouse human recombinant IL-15 was administered every three days via the I.P. route to provide NK-cell support. Table 4 summarizes the groups of mice treated in the study.

Bioluminescence imaging (BLI) was performed twice per week to monitor tumor burden. Mice were checked daily for signs of discomfort and tolerability, and body weight was measured twice per week beginning one week after tumor inoculation. Mice were imaged after 15 minutes of subcutaneous injection of 150 mg/kg D-luciferin. Total flux (photons/second) of the entire mouse was quantified using Living Image software (PerkinElmer). Tumor bearing mice were sacrificed upon development of symptomatic myeloma, such as hind limb paralysis, grooming, and/or lethargy. Time to sacrifice was used as a proxy for survival. All surviving mice were sacrificed 43 days after initial NK-cell dose for tissue collection. At the completion of the study, flow cytometry was used to quantify g-NK, cNK, and MM.1S (CD138pos/CD45neg) cells from biological samples to determine tumor burden and NK-cell survival.

TABLE E4
MM Efficacy Study Design

| Group | | Number of | Days of Antibody | Days of NK cell |
|---|---|---|---|---|
| Number | Arm | Mice | Administration | administration |
| 1 | Vehicle control | 8 | N/A | N/A |
| 2 | g-NK I.V. + 10 ug | 7 | 14, 21, 28, 35, 42 | 14, 21, 28, 35, 42 |
| | Daratumumab I.P. + | | | |
| | IL-15 I.P.* | | | |
| 3 | cNK I.V. + 10 ug | 7 | 14, 21, 28, 35, 42 | 14, 21, 28, 35, 42 |
| | Daratumumab I.P. + | | | |
| | IL-15 I.P.* | | | |

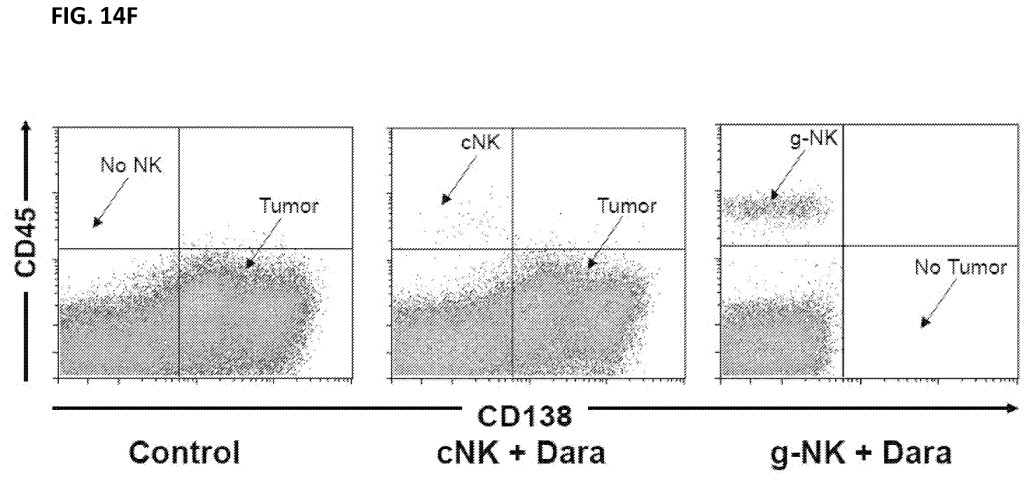

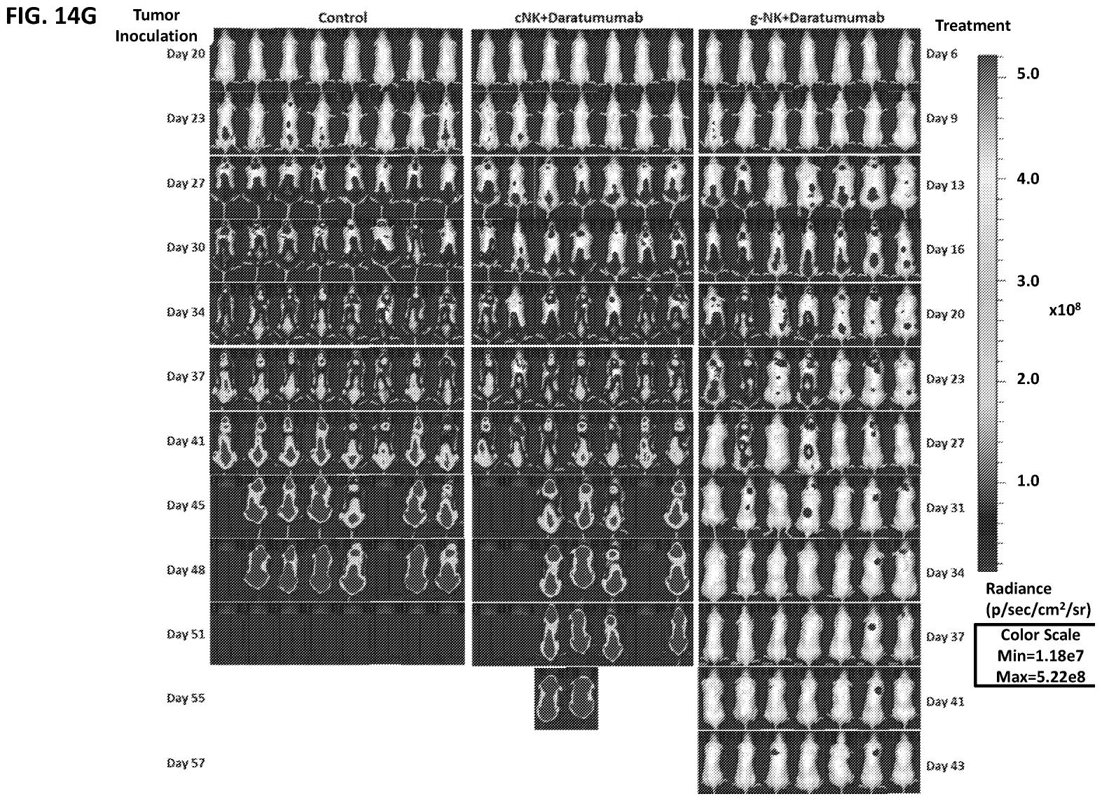

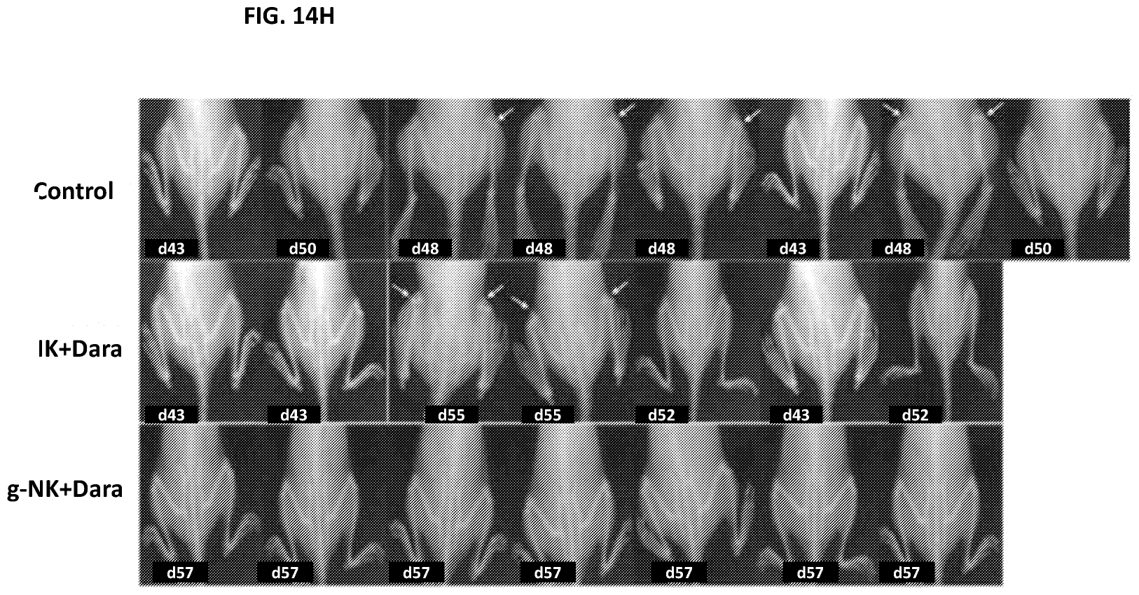

Co-administration of g-NK and daratumumab resulted in significant tumor inhibition and enhanced survival compared to treatment with cNK and daratumumab. As shown in FIG. 14A, g-NK cells plus daratumumab eliminated myeloma tumor burden in 5 of 7 mice evidenced by BLI imaging after 5 weeks of treatment. Quantitative BLI analysis showed g-NK plus daratumumab induced sustained and statistically significant tumor regression (FIG. 14B). The Kaplan-Meier survival analysis showed that the overall survival probability of the g-NK plus daratumumab treated mice was significantly better than those mice treated with vehicle or with cNK and daratumumab (p<0.0001) (FIG. 14C). All mice dosed with g-NK cells were energetic with no weight loss or toxicities observed at the conclusion of the study, while all control mice or mice treated with cNK cells and daratumumab had severe weight loss and succumbed to myeloma before conclusion of the study (FIG. 14D). Interestingly, one of the mice treated with g-NK cells was not dosed until day 21 after tumor inoculation due to anesthesia-induced suffocation of one of the mice, and this mouse had no detectable tumor BLI at the conclusion of the study despite having the highest peak BLI of the g-NK mice (FIG. 14A, mouse labeled as #). Of the 7 mice who were dosed with g-NK cells, only 2 had a minimally detectable amount of residual tumor BLI.

Flow cytometry analysis of the bone marrow confirmed that the 5 g-NK treated mice with no detectable tumor BLI were in fact tumor free (no CD138 pos cell in bone marrow). The average tumor burden for all 7 g-NK treated mice was reduced greater than 99/a relative to mice treated with cNK and daratumumab (p<0.001; FIG. 14E). Representative flow cytometry dot plots depicting tumor burden and persistent NK-cells in bone marrow are shown in FIG. 14F. All of the BLI images taken over the course of the study are shown in FIG. 14G. X-ray images were obtained from all of the mice prior to sacrifice and it was determined that control mice or mice treated with cNK cells and daratumumab had fractures and malformations of the hind limb bones, while one of the mice treated with g-NK cells and daratumumab had any bone deformities (FIG. 14H).

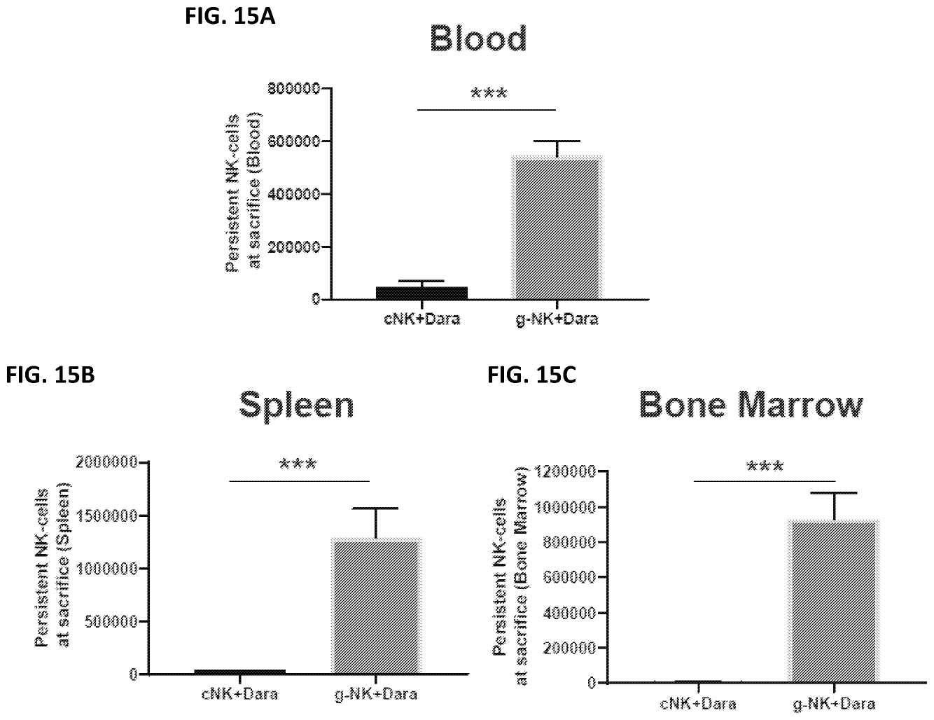

Analysis of NK cells in blood, spleen and bone marrow demonstrated a large increased in persistence of g-NK cells in daratumumab treated mice relative to cNK cells (FIG. 15A-C). Notably, g-NK cell numbers were >90% higher than cNK cells in blood (FIG. 15A), >95% higher in spleen (FIG. 15B), and >99% higher in bone marrow (FIG. 15C).

Taken together, the results further support the superiority of g-NK cells, including compared to cNK cells, for enhancing mAb effects in vivo and suggest that g-NK cells given in combination with daratumumab could be potentially curative for MM. Further, the results support that enhanced survival and resistance to fratricide result in superior anti-tumor effects and persistence of g-NK cells.

### Example 11: Identification of g-NK Surrogate Surface Markers

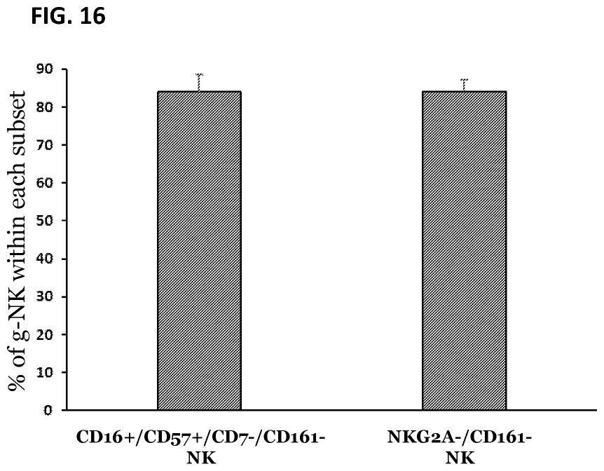

A study was carried out to identify a combination of extracellular surface markers that could be used as surrogate surface markers to identify g-NK cells, which are negative for the intracellular marker FcεR1γ (FcRγ^{neg}). The percentage of g-NK cells were determined in a human peripheral blood sample by flow cytometry by intracellular staining for FcεR1γ and by extracellular staining for CD45, CD3 and CD56 to identify the g-NK cell subset CD45^{pos}/CD3^{neg}/CD56^{pos}/FcRγ^{neg}. As shown in FIG. 16, among g-NK cells in the sample, cells having the NK cell phenotype CD45^{pos}/CD3^{neg}/CD56^{pos} and that had an extracellular surface phenotype of CD16^{pos}/CD57^{pos}/CD7^{dim/neg}/CD161^{neg} or NKG2A^{neg}/CD161^{neg} highly correlated to the presence of g-NK cells in the sample. Specifically, the percentage of g-NK cells within the CD16^{pos}/CD57^{pos}/CD7^{dim/neg}/CD161^{neg} or NKG2A^{neg}/CD161^{neg} NK cell subsets were both greater than 80%.

### Example 12: Transduction Feasibility and Cytotoxic Efficacy of g-NK Cells with a CD20 CAR

A study was carried out to assess expression and potency of a chimeric antigen receptor (CAR) engineered in a g-NK cell. G-NK cells were expanded from enriched CD3^{neg}/CD57^{pos} NK cells from peripheral blood by co-culture with irradiated 221.AEH feeder cells at a 2:1 221.AEH to NK cell ratio in the presence of 500 IU/mL of IL-2, 10 ng/mL of IL-15, and 25 ng/mL of IL-21, substantially as described in Example 5. The cells were electroporated using the Neon™ transfection system. Cells were washed with PBS prior to electroporation and re-suspended in Opti-MEM™ medium at a cell density of 4.8×107/mL. 100 μL of cells were mixed with 14.4 μg of 1 mg/mL GFP mRNA or CAR-CD20 mRNA. The CAR-CD20 was composed of murine anti-CD20 (Leu16) scFv that binds a CD20 polypeptide (SEQ ID NO: 37), an IgG4 Fc spacer (SEQ ID NO:38), a CD28 transmembrane domain (SEQ ID NO:39), a CD28 intracellular co-stimulatory signaling domain (SEQ ID NO:39), and a CD3ζ primary signaling domain (SEQ ID NO:41) The amino acid sequence of the CAR is set forth in SEQ ID NO: 42 (GenBank No. KX055829.1), and also was generated with an HA tag at the 5′-end. The anti-CD20 mRNA is set forth in SEQ ID NO: 45.

The mixtures of g-NK cells and mRNA were subjected to electroshock at a first pulse of 1820 (20 ms) followed by a second pulse of 500V (100 ms). Cells were then cultured in 2 mL of NK MACS® media with 500 IU/mL of IL-2, 10 ng/mL of IL-15, and 25 ng/mL of IL-21. The cells were incubated for 24 hours at 37° C. with 5% CO₂.

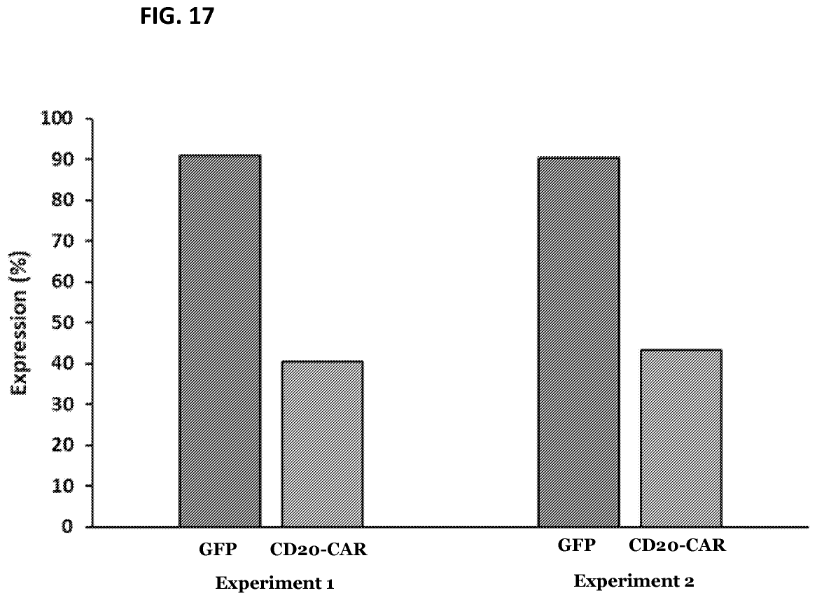

After the incubation, the cells were analyzed by flow cytometry to evaluate the levels of GFP and CAR-CD20 expression. The post-transduction expressions of GFP and CD20-CAR by g-NK cells from two separate experiments using distinct donors are shown in FIG. 17. The percentage of GFP or CAR-CD20-positive cells is reported as the percentage of surviving cells expressing GFP or CAR-CD20.

Cytotoxicity of g-NK cells (effector) with or without the CAR-CD20 was analyzed by incubating the cells with 60,000 Raji lymphoma cells (target) at various effector-to-target (E:T) ratios in the presence or absence of rituximab (Rituxan®). The concentration of rituximab (Rituxan®) used was 5 μg/ml. Raji cells were pre-stained with APC-conjugated mouse anti-human CD19 mAb. Residual dye was removed by washing with RPMI 1640 medium with 10% FBS and 1% penicillin-streptomycin (assay medium). The pre-stained Raji cells were verified by flow cytometry to confirm that all target cells were successfully labeled.

Day 17 expanded g-NK cells or 36-hour post electroporation CAR-CD20 g-NK cells were mixed with the prestained Raji target cells at the E:T ratios 0.5:1, 2.5:1, and 5:1 in 1 mL of assay medium. The mixtures were pelleted at 300 g for 5 minutes and then incubated for 4 hours at 37° C. with 5% CO₂. Cytotoxicity was assessed using 4-color flow cytometry. To identify NK cells (CD3^{neg}/CD56^{pos}), PE-conjugated mouse anti-human CD56 mAb and FITC-conjugated mouse anti-human CD3 mAb were used. To distinguish live (CD19^{pos}/PI^{neg}) and dead target (CD19^{pos}/PI^{pos}) cells, propidium iodide (PI) was used.

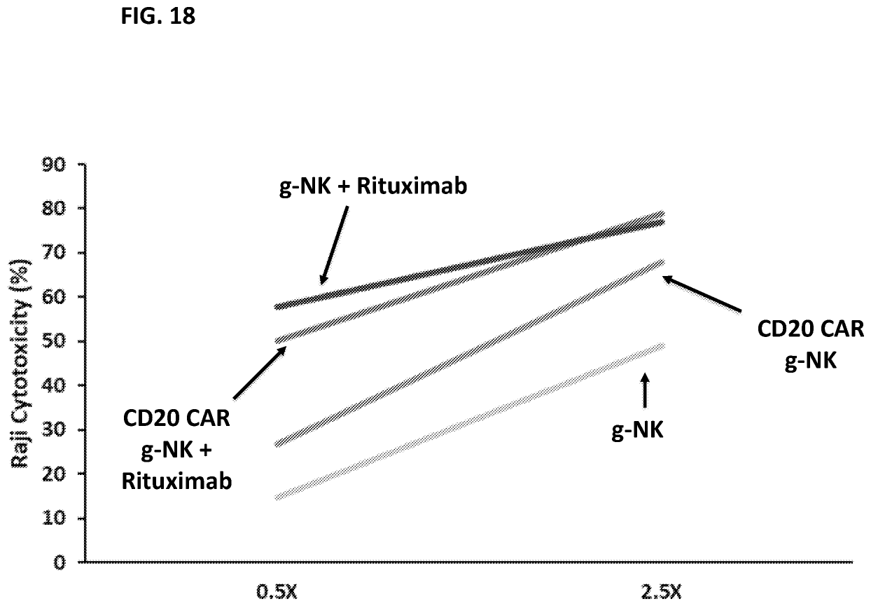

FIG. 18 demonstrates the potency of g-NK cells with or without the CD20-CAR against Raji lymphoma cells in the presence or absence of rituximab (Rituxan®). Addition of the CD20-CAR enhances the potency of the g-NK cells as a monotherapy. Addition of rituximab (Rituxan) also enhanced the potency of the g-NK cells to similar levels, whether or not the CD20-CAR is expressed.

This result demonstrates that the CAR is functional in g-NK cells that are deficient in expression of FcRγ chain. This result further confirms the surprising finding that addition of the CAR does not undermine g-NK cell ADCC mechanism via CD16-engagement by an IgG1 mAb. Thus, in addition to activity of the CAR-engineered g-NK cell monotherapy, these results support a combination therapy with g-NK cell engineered with a CAR and an Fc-targeted agent, such as an IgG1 mAb, including approaches using CAR targeting antigens distinct from the mAb. Such dual-targeting strategies could potentially be functionally additive or compensatory depending on the antigen expression of a tumor and possible antigen loss.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequence Table

| # | SEQUENCE | ANNOTATION |
|---|---|---|
| 1 | APTSSSTKKTQLQLEHLLLDLQMILNGINNYKNPKLTRM | Human IL-2 (mature; |
| | LTFKFYMPKKATELKHLQCLEEELKPLEEVLNLAQSKNF | UniProt P60568) |
| | HLRPRDLISNINVIVLELKGSETTFMCEYADETATIVEFLN | |
| | RWITFCQSIISTLT | |
| 2 | NWVNVISDLKKIEDLIQSMHIDATLYTESDVHPSCKVTA | Human IL-15 (mature; |
| | MKCFLLELQVISLESGDASIHDTVENLIILANNSLSSNGN | UniProt P40933) |
| | VTESGCKECEELEEKNIKEFLQSFVHIVQMFINTS | |
| 3 | HKSSSQGQDRHMIRMRQLIDIVDQLKNYVNDLVPEFLPA | Human IL-21 (mature; |
| | PEDVETNCEWSAFSCFQKAQLKSANTGNNERIINVSIKK | UniProt Q9HBE4) |
| | LKRKPPSTNAGRRQKHRLTCPSCDSYEKKPPKEFLERFK | |
| | SLLQKMIHQHLSSRTHGSEDS | |
| 4 | QGQDRHMIRMRQLIDIVDQLKNYVNDLVPEFLPAPEDV | IL-21 |
| | ETNCEWSAFSCFQKAQLKSANTGNNERIINVSIKKLKRK | |
| | PPSTNAGRRQKHRLTCPSCDSYEKKPPKEFLERFKSLLQ | |
| | KMIHQHLSSRTHGSEDS | |
| 5 | MLLLVTSLLLCELPHPAFLLIPNWVNVISDLKKIEDLIQS | Membrane-bound IL- |
| | MHIDATLYTESDVHPSCKVTAMKCFLLELQVISLESGDA | 15 (mbIL-15) |
| | SIHDTVENLIILANNSLSSNGNVTESGCKECEELEEKNIKE | |
| | FLQSFVHIVQMFINTSESKYGPPCPPCPAPEFLGGPSVFLF | |
| | PPKPKDTLMISRTPEVTCVVVDVSQEDPEVQFNWYVDG | |
| | VEVHNAKTKPREEQFNSTYRVVSVLTVLHQDWLNGKE | |
| | YKCKVSNKGLPSSIEKTISKAKGQPREPQVYTLPPSQEEM | |
| | TKNQVSLTCLVKGFYPSDIAVEWESNGQPENNYKTTPPV | |
| | LDSDGSFFLYSRLTVDKSRWQEGNVFSCSVMHEALHNH | |
| | YTQKSLSLSLGKMALIVLGGVAGLLLFIGLGIFF | |
| 6 | MLLLVTSLLLCELPHPAFLLIPQDRHMIRMRQLIDIVDQL | Membrane-bound IL- |
| | KNYVNDLVPEFLPAPEDVETNCEWSAFSCFQKAQLKSA | 21 (mbIL-21) |
| | NTGNNERIINVSIKKLKRKPPSTNAGRRQKHRLTCPSCDS | |
| | YEKKPPKEFLERFKSLLQKVSTLSFIESKYGPPCPPCPAPE | |
| | FLGGPSVFLFPPKPKDTLMISRTPEVTCVVVDVSQEDPEV | |
| | QFNWYVDGVEVHNAKTKPREEQFNSTYRVVSVLTVLH | |
| | QDWLNGKEYKCKVSNKGLPSSIEKTISKAKGQPREPQVY | |
| | TLPPSQEEMTKNQVSLTCLVKGFYPSDIAVEWESNGQPE | |
| | NNYKTTPPVLDSDGSFFLYSRLTVDKSRWQEGNVFSCSV | |
| | MHEALHNHYTQKSLSLSLGKMALIVLGGVAGLLLFIGL | |
| | GIFF | |
| 7 | MALPVTALLLPLALLLHAARPNWVNVISDLKKIEDLIQS | Membrane-bound IL- |
| | MHIDATLYTESDVHPSCKVTAMKCFLLELQVISLESGDA | 15 (mbIL-15) |
| | SIHDTVENLIILANNSLSSNGNVTESGCKECEELEEKNIKE | |
| | FLQSFVHIVQMFINTSTTTPAPRPPTPAPTIASQPLSLRPEA | |
| | CRPAAGGAVHTRGLDFACDIYIWAPLAGTCGVLLLSLVI | |
| | TLYC | |
| 8 | MALPVTALLLPLALLLHAARPNWVNVISDLKKIEDLIQS | Membrane-bound IL- |
| | MHIDATLYTESDVHPSCKVTAMKCFLLELQVISLESGDA | 15 (mbIL-15) |
| | SIHDTVENLIILANNSLSSNGNVTESGCKECEELEEKNIKE | |
| | FLQSFVHIVQMFINTSTTTPAPRPPTPAPTIASQPLSLRPEA | |
| | CRPAAGGAVHTRGLDFACDIYIWAPLAGTCGVLLLSLVS | |
| | PFTA | |
| 9 | ATGAGAATTTCGAAACCACATTTGAGAAGTATTTCCA | IL-15 nucleotide |
| | TCCAGTGCTACTTGTGTTTACTTCTAAACAGTCATTTT | |
| | CTAACTGAAGCTGGCATTCATGTCTTCATTTTGGGCTG | |
| | TTTCAGTGCAGGGCTTCCTAAAACAGAAGCCAACTGG | |
| | GTGAATGTAATAAGTGATTTGAAAAAAATTGAAGATC | |
| | TTATTCAATCTATGCATATTGATGCTACTTTATATACG | |
| | GAAAGTGATGTTCACCCCAGTTGCAAAGTAACAGCAA | |
| | TGAAGTGCTTTCTCTTGGAGTTACAAGTTATTTCACTT | |
| | GAGTCCGGAGATGCAAGTATTCATGATACAGTAGAAA | |
| | ATCTGATCATCCTAGCAAACAACAGTTTGTCTTCTAAT | |
| | GGGAATGTAACAGAATCTGGATGCAAAGAATGTGAG | |
| | GAACTGGAGGAAAAAAATATTAAAGAATTTTTGCAGA | |
| | GTTTTGTACATATTGTCCAAATGTTCATCAACACTTCT | |
| | TGA | |
| 10 | IEVMYPPPYLDNEKSNGTIIHVKGKHLCPSPLFPGPSKPF | CD28 transmembrane |
| | WVLVVVGGVLACYSLLVTVAFIIFWVR | domain |
| 11 | TTTPAPRPPTPAPTIASQPLSLRPEACRPAAGGAVHTRGL | CD8 transmembrane |
| | DFACDIYIWAPLAGTCGVLLLSLVITLYC | domain |
| 12 | MALPVTALLLPLALLLHAARP | CD8alpha signal |
| | | peptide |
| 13 | MLLLVTSLLLCELPHPAFLLIP | GM-CSFRa signal |
| | | peptide |
| 14 | MRLPAQLLGLLMLWVPGSSG | IgK signal peptide |
| 15 | MALIVLGGVAGLLLFIGLGIFF | CD4 transmembrane |
| | | domain |
| 16 | UAAUUUCUACUCUUGUAGAU | Cpfl crRNA scaffold |
| | | sequence |
| 17 | ATATTTACAGAATGGCACAGG | primer |
| 18 | GACTTGGTACCCAGGTTGAA | Primer |
| 19 | ATCAGATTCGATCCTACTTCTGCAGGGGGCAT | Primer |
| 20 | ACGTGCTGAGCTTGAGTGATGGTGATGTTCAC | Primer |
| 21 | CCCAACTCAACTTCCCAGTGTGAT | Primer |
| 22 | GAAATCTACCTTTTCCTCTAATAGGGCAAT | Primer |
| 23 | GAAATCTACCTTTTCCTCTAATAGGGCAA | Primer |
| 24 | GAAATCTACCTTTTCCTCTAATAGGGCA | Primer |
| 25 | CCAAAAGCCACACTCAAAGAC | Primer |
| 26 | ACCCAGGTGGAAAGAATGATG | Primer |
| 27 | AACATCACCATCACTCAAGGTTTGG | Primer |
| 28 | CTGAAGACACATTTTTACTCCCAAA | Primer |
| 29 | TCCAAAAGCCACACTCAAAGAC | Primer |
| 30 | CTGAAGACACATTTTTACTCCCAAC | Primer |
| 31 | GMRTEDLPKAVVFLEPQWYRVLEKDSVTLKCQGAYSPE | CD16 (F158) |
| | DNSTQWFHNESLISSQASSYFIDAATVDDSGEYRCQTNL | |
| | STLSDPVQLEVHIGWLLLQAPRWVFKEEDPIHLRCHSWK | |
| | NTALHKVTYLQNGKGRKYFHHNSDFYIPKATLKDSGSY | |
| | FCRGLFGSKNVSSETVNITITQGLAVSTISSFFPPGYQVSF | |
| | CLVMVLLFAVDTGLYFSVKTNIRSSTRDWKDHKFKWRK | |
| | DPQDK | |
| 32 | MWQLLLPTALLLLVSA | Signal peptide |
| 33 | GMRTEDLPKAVVFLEPQWYRVLEKDSVTLKCQGAYSPE | CD16 (V158 or |
| | DNSTQWFHNESLISSQASSYFIDAATVDDSGEYRCQTNL | 158V+) |
| | STLSDPVQLEVHIGWLLLQAPRWVFKEEDPIHLRCHSWK | |
| | NTALHKVTYLQNGKGRKYFHHNSDFYIPKATLKDSGSY | |
| | FCRGLVGSKNVSSETVNITITQGLAVSTISSFFPPGYQVSF | |
| | CLVMVLLFAVDTGLYFSVKTNIRSSTRDWKDHKFKWRK | |
| | DPQDK | |
| 34 | MIPAVVLLLLLLVEQAAALGEPQLCYILDAILFLYGIVLT | FcRγ |
| | LLYCRLKIQVRKAAITSYEKSDGVYTGLSTRNQETYETL | |
| | KHEKPPQ | |
| 35 | IVLTQSPAILSASPGEKVTMTCRASSSVNYMDWYQKKPG | Anti-CD20 (Leu16) |
| | SSPKPWIYATSNLASGVPARFSGSGSGTSYSLTISRVEAE | VL |
| | DAATYYCQQWSFNPPTFGGGTKLEIK | |
| 36 | GAELVKPGASVKMSCKASGYTFTSYNMHWVKQTPGQG | Anti-CD20 (Leu16) |
| | LEWIGAIYPGNGDTSYNQKFKGKATLTADKSSSTAYMQ | VH |
| | LSSLTSEDSADYYCARSNYYGSSYWFFDVWGAGTTVTV | |
| | SSLD | |
| 37 | IVLTQSPAILSASPGEKVTMTCRASSSVNYMDWYQKKPG | Anti-CD20 (Leu16) |
| | SSPKPWIYATSNLASGVPARFSGSGSGTSYSLTISRVEAE | scFv |
| | DAATYYCQQWSFNPPTFGGGTKLEIKGSTSGGGSGGGS | |
| | GGGGSSEVQLQQSGAELVKPGASVKMSCKASGYTFTSY | |
| | NMHWVKQTPGQGLEWIGAIYPGNGDTSYNQKFKGKAT | |
| | LTADKSSSTAYMQLSSLTSEDSADYYCARSNYYGSSYW | |
| | FFDVWGAGTTVTVSSLD | |
| 38 | ESKYGPPCPPCPAPEFLGGPSVFLFPPKPKDTLMISRTPEV | IgG4 Fc spacer |
| | TCVVVDVSQEDPEVQFNWYVDGVEVHNAKTKPREEQF | |
| | NSTYRVVSVLTVLHQDWLNGKEYKCKVSNKGLPSSIEK | |
| | TISKAKGQPREPQVYTLPPSQEEMTKNQVSLTCLVKGFY | |
| | PSDIAVEWESNGQPENNYKTTPPVLDSDGSFFLYSRLTV | |
| | DKSRWQEGNVFSCSVMHEALHNHYTQKSLSLSLGKM | |
| 39 | FWVLVVVGGVLACYSLLVTVAFIIFWV | CD28 transmembrane |
| | | domain |
| 40 | RSKRSRGGHSDYMNMTPRRPGPTRKHYQPYAPPRDFAA | CD28 costimulatory |
| | YRS | signaling domain |
| 41 | RVKFSRSADAPAYQQGQNQLYNELNLGRREEYDVLDK | CD3 zeta signaling |
| | RRGRDPEMGGKPRRKNPQEGLYNELQKDKMAEAYSEIG | domain |
| | MKGERRRGKGHDGLYQGLSTATKDTYDALHMQALPPR | |
| 42 | IVLTQSPAILSASPGEKVTMTCRASSSVNYMDWYQKKPG | Anti-CD20 CAR |
| | SSPKPWIYATSNLASGVPARFSGSGSGTSYSLTISRVEAE | Genbank No. |
| | DAATYYCQQWSFNPPTFGGGTKLEIKGSTSGGGSGGGS | KX055829.1 |
| | GGGGSSEVQLQQSGAELVKPGASVKMSCKASGYTFTSY | |
| | NMHWVKQTPGQGLEWIGAIYPGNGDTSYNQKFKGKAT | |
| | LTADKSSSTAYMQLSSLTSEDSADYYCARSNYYGSSYW | |
| | FFDVWGAGTTVTVSSLDESKYGPPCPPCPAPEFLGGPSV | |
| | FLFPPKPKDTLMISRTPEVTCVVVDVSQEDPEVQFNWYV | |
| | DGVEVHNAKTKPREEQFNSTYRVVSVLTVLHQDWLNG | |
| | KEYKCKVSNKGLPSSIEKTISKAKGQPREPQVYTLPPSQE | |
| | EMTKNQVSLTCLVKGFYPSDIAVEWESNGQPENNYKTT | |
| | PPVLDSDGSFFLYSRLTVDKSRWQEGNVFSCSVMHEAL | |
| | HNHYTQKSLSLSLGKMFWVLVVVGGVLACYSLLVTVA | |
| | FIIFWVRSKRSRGGHSDYMNMTPRRPGPTRKHYQPYAPP | |
| | RDFAAYRSRVKFSRSADAPAYQQGQNQLYNELNLGRRE | |
| | EYDVLDKRRGRDPEMGGKPRRKNPQEGLYNELQKDKM | |
| | AEAYSEIGMKGERRRGKGHDGLYQGLSTATKDTYDAL | |
| | HMQALPPR | |
| 43 | METDTLLLWVLLLWVPGSTGD | IgK Signal peptide |
| 44 | YPYDVPDYA | HA tag |
| 45 | atggagacagacacactcctgctatgggtgctgctgctctgggttccaggttccacaggtg | Anti-CD20 CAR |
| | actacccatacgatgttccagattacgctattgtgctgacccaatctccagctatcctgtctg | (for Trilink RNA) |
| | catctccaggggagaaggtcacaatgacttgcagggccagctcaagtgtaaattacatgg | |
| | actggtaccagaagaagccaggatcctcccccaaaccctggatttatgccacatccaacc | |
| | tggcttctggagtccctgctcgcttcagtggcagtgggtctgggacctcttactctctcaca | |
| | atcagcagagtggaggctgaagatgctgccacttattactgccagcagtggagttttaatc | |
| | cacccacgttcggaggggggaccaagctggaaataaaaggcagtactagcggtggtgg | |
| | ctccgggggcggttccggtgggggcggcagcagcgaggtgcagctgcagcagtctgg | |
| | ggctgagctggtgaagcctggggcctcagtgaagatgtcctgcaaggcttctggctacac | |
| | atttaccagttacaatatgcactgggtaaagcagacacctggacagggcctggaatggatt | |
| | ggagctatttatccaggaaatggtgatacttcctacaatcagaagttcaaaggcaaggcca | |
| | cattgactgcagacaaatcctccagcacagcctacatgcagctcagcagcctgacatctg | |
| | aggactctgcggactattactgtgcaagatctaattattacggtagtagctactggttcttcg | |
| | atgtctggggcgcagggaccacggtcaccgtctcctcactcgacgaatctaagtacgga | |
| | ccgccctgccccccttgccctgcccccgagttcctgggggacccagcgtgttcctgttc | |
| | ccccccaagcccaaggacaccctgatgatcagccggacccccgaggtgacctgcgtgg | |
| | tggtggacgtgagccaggaagatcccgaggtccagttcaattggtacgtggacggcgtg | |
| | gaagtgcacaacgccaagaccaagcccagagaggaacagttcaacagcacctaccgg | |
| | gtggtgtctgtgctgaccgtgctgcaccaggactggctgaacggcaaagaatacaagtg | |
| | caaggtgtccaacaagggcctgcccagcagcatcgaaaagaccatcagcaaggccaa | |
| | gggccagcctcgcgagccccaggtgtacaccctgcctccccccaggaagagatgacc | |
| | aagaaccaggtgtccctgacctgcctggtgaagggcttctaccccagcgacatcgccgt | |
| | ggagtgggagagcaacggccagcctgagaacaactacaagaccacccctcccgtgct | |
| | ggacagcgacggcagcttcttcctgtacagccggctgaccgtggacaagagccggtgg | |
| | caggaaggcaacgtctttagctgcagcgtgatgcacgaggccctgcacaaccactacac | |
| | ccagaagagcctgagcctgtccctgggcaagatgttctgggtgctggtggtggtgggcg | |
| | gggtgctggcctgctacagcctgctggtgacagtggccttcatcatcttttgggtgcggag | |
| | caagcggagcagaggcggccacagcgactacatgaacatgacccccagacggcctgg | |
| | ccccacccggaagcactaccagccctacgccccacccagggactttgccgcctacaga | |
| | agccgggtgaagttcagcagaagcgccgacgcccctgcctaccagcagggccagaat | |
| | cagctgtacaacgagctgaacctgggcagaagggaagagtacgacgtcctggataagc | |
| | ggagaggccgggaccctgagatgggcggcaagcctcggcggaagaacccccaggaa | |
| | ggcctgtataacgaactgcagaaagacaagatggccgaggcctacagcgagatcggca | |
| | tgaagggcgagcggaggggggcaagggccacgacggcctgtatcagggcctgtcc | |
| | accgccaccaaggatacctacgacgccctgcacatgcaggccctgcccccaaggtga | |
| 46 | QVQLVQSGAEVKKPGSSVKVSCKAFGGTFSSYAISWVR | Anti-CD38 VH |
| | QAPGQGLEWMGRIIRFLGIANYAQKFQGRVTLIADKSTN | |
| | TAYMELSSLRSEDTAVYYCAGEPGERDPDAVDIWGQGT | |
| | MVTVSS | |
| 47 | EVQLVQSGAEVKKPGESLKISCKGSGYSFSNYWIGWVR | Anti-CD38 VH |
| | QMPGKGLEWMGIIYPHDSDARYSPSFQGQVTFSADKSIS | |
| | TAYLQWSSLKASDTAMYYCARHVGWGSRYWYFD | |
| | LWGRGTLVTVSS | |
| 48 | EIVLTQSPDFQSVTPKEKVTITCRASQSIGSSLHWYQQKP | Anti-CD38 VL |
| | DQSPKLLIKYASQSFSGVPSRFSGSGSGTDFTLTINSLEAE | |
| | DAATYYCHQSSSLPYTFGQGTKLEIK | |
| 49 | DIQMTQSPSSLSASVGDRVTITCRASQGISNYLAWFQQK | Anti-CD38 VL |
| | PGKAPKSLIYAASSLQSGVPSRFSGSGSGTDFTLTISSLQP | |
| | EDFATYYCQQYNSYPYTFGQGTKLEIK | |
| 50 | RVKFSRSADAPAYKQGQNQLYNELNLGRREEYDVLDK | CD3 zeta |
| | RRGRDPEMGGKPRRKNPQEGLYNELQKDKMAEAYSEIG | |
| | MKGERRRGKGHDGLYQGLSTATKDTYDALHMQALPPR | |
| 51 | KRGRKKLLYIFKQPFMRPVQTTQEEDGCSCRFPEEEEGG | 4-1BB costimulatory |
| | CEL | domain |
| 52 | RSKRSRLLHSDYMNMTPRRPGPTRKHYQPYAPPRDFAA | CD28 costimulatory |
| | YRS | domain |
| 53 | DIQMTQTTSSLSASLGDRVTISCRASQDISKYLNWYQQK | Anti-CD19 VL |
| | PDGTVKLLIYHTSRLHSGVPSRFSGSGSGTDYSLTISNLE | |
| | QEDIATYFCQQGNTLPYTFGGGTKLEIT | |
| 54 | EVKLQESGPGLVAPSQSLSVTCTVSGVSLPDYGVSWIRQ | Anti-CD19 VH |
| | PPRKGLEWLGVIWGSETTYYNSALKSRLTIIKDNSKSQV | |
| | FLKMNSLQTDDTAIYYCAKHYYYGGSYAMDYWGQGTS | |
| | VTVSS | |
| 55 | GSTSGSGKPGSGEGSTKG | Whitlow linker |
| 56 | GGGGSGGGGSGGGGS | GS linker |
| 57 | DIQMTQTTSSLSASLGDRVTISCRASQDISKYLNWYQQK | Anti-CD19 scFv |
| | PDGTVKLLIYHTSRLHSGVPSRFSGSGSGTDYSLTISNLE | |
| | QEDIATYFCQQGNTLPYTFGGGTKLEITGSTSGSGKPGSG | |
| | EGSTKGEVKLQESGPGLVAPSQSLSVTCTVSGVSLPDYG | |
| | VSWIRQPPRKGLEWLGVIWGSETTYYNSALKSRLTIIKD | |
| | NSKSQVFLKMNSLQTDDTAIYYCAKHYYYGGSYAMDY | |
| | WGQGTSVTVSS | |
| 58 | DIQMTQTTSSLSASLGDRVTISCRASQDISKYLNWYQQK | Anti-CD19 scFv |
| | PDGTVKLLIYHTSRLHSGVPSRFSGSGSGTDYSLTISNLE | |
| | QEDIATYFCQQGNTLPYTFGGGTKLEITGGGGSGGGGSG | |
| | GGGSEVKLQESGPGLVAPSQSLSVTCTVSGVSLPDYGVS | |
| | WIRQPPRKGLEWLGVIWGSETTYYNSALKSRLTIIKDNS | |
| | KSQVFLKMNSLQTDDTAIYYCAKHYYYGGSYAMDYW | |
| | GQGTSVTVSS | |
| 59 | ESKYGPPCPPCP | IgG4 hinge |
| 60 | TTTPAPRPPTPAPTIASQPLSLRPE | CD8 hinge |
| 61 | ACRPAAGGAVHTRGLDFACDIYIWAPLAGTCGVLLLSL | CD8 transemembrane |
| | VITLYC | |
| 62 | DIVLTQSPASLAMSLGKRATISCRASESVSVIGAHLIHWY | Anti-BCMA C11D5.3 |
| | QQKPGQPPKLLIYLASNLETGVPARFSGSGSGTDFTLTID | VL |
| | PVEEDDVAIYSCLQSRIFPRTFGGGTKLEIK | |
| 63 | QIQLVQSGPELKKPGETVKISCKASGYTFTDYSINWVKR | Anti-BCMA C11D5.3 |
| | APGKGLKWMGWINTETREPAYAYDFRGRFAFSLETSAS | VH |
| | TAYLQINNLKYEDTATYFCALDYSYAMDYWGQGTSVT | |
| | VSS | |
| 64 | DIVLTQSPASLAMSLGKRATISCRASESVSVIGAHLIHWY | Anti-BCMA C11D5.3 |
| | QQKPGQPPKLLIYLASNLETGVPARFSGSGSGTDFTLTID | scFv |
| | PVEEDDVAIYSCLQSRIFPRTFGGGTKLEIKGSTSGSGKP | |
| | GSGEGSTKGQIQLVQSGPELKKPGETVKISCKASGYTFT | |
| | DYSINWVKRAPGKGLKWMGWINTETREPAYAYDFRGR | |
| | FAFSLETSASTAYLQINNLKYEDTATYFCALDYSYAMDY | |
| | WGQGTSVTVSS | |
| 65 | DIVLTQSPPSLAMSLGKRATISCRASESVTILGSHLIYWY | Anti-BCMA C12A3.2 |
| | QQKPGQPPTLLIQLASNVQTGVPARFSGSGSRTDFTLTID | VL |
| | PVEEDDVAVYYCLQSRTIPRTFGGGTKLEIK | |
| 66 | QIQLVQSGPELKKPGETVKISCKASGYTFRHYSMNWVK | Anti-BCMA C12A3.2 |
| | QAPGKGLKWMGRINTESGVPIYADDFKGRFAFSVETSAS | VH |
| | TAYLVINNLKDEDTASYFCSNDYLYSLDFWGQGTALTV | |
| | SS | |
| 67 | DIVLTQSPPSLAMSLGKRATISCRASESVTILGSHLIYWY | Anti-BCMA C12A3.2 |
| | QQKPGQPPTLLIQLASNVQTGVPARFSGSGSRTDFTLTID | scFv |
| | PVEEDDVAVYYCLQSRTIPRTFGGGTKLEIKGSTSGSGKP | |
| | GSGEGSTKGQIQLVQSGPELKKPGETVKISCKASGYTFR | |
| | HYSMNWVKQAPGKGLKWMGRINTESGVPIYADDFKGR | |
| | FAFSVETSASTAYLVINNLKDEDTASYFCSNDYLYSLDF | |
| | WGQGTALTVSS | |
| 68 | QIQLVQSGPELKKPGETVKISCKASGYTFTDYSINWVKR | Anti-BCMA BCMA02 |
| | APGKGLKWMGWINTETREPAYAYDFRGRFAFSLETSAS | VH |
| | TAYLQINNLKYEDTATYFCALDYSYAMDYWGQG | |
| | TSVTVSS | |
| 69 | DIVLTQSPPSLAMSLGKRATISCRASESVTILGSHLIHWY | Anti-BCMA BCMA02 |
| | QQKPGQPPTLLIQLASNVQTGVPARFSGSGSRTDFTLTID | VL |
| | PVEEDDVAVYYCLQSRTIPRTFGGGTKLEIK | |
| 70 | DIVLTQSPPSLAMSLGKRATISCRASESVTILGSHLIHWY | Anti-BCMA02 scFv |
| | QQKPGQPPTLLIQLASNVQTGVPARFSGSGSRTDFTLTID | |
| | PVEEDDVAVYYCLQSRTIPRTFGGGTKLEIKGSTSGSGKP | |
| | GSGEGSTKGQIQLVQSGPELKKPGETVKISCKASGYTFT | |
| | DYSINWVKRAPGKGLKWMGWINTETREPAYAYDFRGR | |
| | FAFSLETSASTAYLQINNLKYEDTATYFCALDYSYAMDY | |
| | WGQGTSVTVSS | |

What is claimed:

1. An engineered Natural Killer (NK) cell that is deficient in expression of FcRγ chain (g-NK cells) and comprises a heterologous nucleic acid encoding a chimeric antigen receptor (CAR).

2. An engineered Natural Killer (NK) cell that is deficient in expression of FcRγ chain (g-NK cells), and comprises a heterologous nucleic acid encoding an immunomodulator.

3. An engineered Natural Killer (NK) cell that is deficient in expression of FcRγ chain (g-NK cells), and comprises a heterologous nucleic acid encoding a chimeric antigen receptor (CAR) and a heterologous nucleic acid encoding an immunomodulator.

4. The engineered NK cell of claim 1 or claim 3, wherein the CAR comprises 1) an antigen binding domain; 2) a flexible linker; 3) a transmembrane region; and 4) an intracellular signaling domain.

5. The engineered NK cell of claim 4, wherein the antigen binding domain is targeted against a tumor antigen.

6. The engineered NK cell of claim 4 or claim 5, wherein the antigen binding domain is a single chain variable fragment (scFv).

7. The engineered NK cell of any of claims 4-6, wherein the intracellular signaling domain comprises a primary signaling domain and a costimulatory signaling domain.

8. The engineered NK cell of any of claims 4-7, wherein the intracellular signaling domain comprises one or more signaling domains of CD3ζ, DAP10, DAP12, CD28, 4-1BB, or OX40.

9. The engineered NK cell of any of claims 4-7, wherein the intracellular signaling domain comprises two or more signaling domains of CD3ζ, DAP10, DAP12, CD28, 4-1BB, or OX40.

10. The engineered NK cell of any of claims 4-9, wherein the intracellular signaling domain comprises a primary signaling domain comprising a signaling domain of CD3ζ and a costimulatory signaling domain.

11. The engineered NK cell of claim 10, wherein the costimulatory signaling domain is a signaling domain of CD28 or 4-1BB.

12. The engineered NK cell of any of claims 1 or 3-11, wherein the heterologous nucleic acid encoding the CAR is stably integrated into the genome of the cell.

13. The engineered NK cell of any of claims 1 or 3-11, wherein the heterologous nucleic acid encoding the CAR is transiently expressed.

14. The engineered NK cell of claim 2 or claim 3, wherein the immunomodulator is an immunosuppressant.

15. The engineered NK cell of claim 2 or claim 3, wherein the immunomodulator is an immunoactivator.

16. The engineered NK cell of any one of claims 2, 3, or 15, wherein the immunomodulator is a cytokine.

17. The engineered NK cell of claim 16, wherein the cytokine is secretable from the engineered NK cell.

18. The engineered NK cell of claim 17, wherein the secretable cytokine is IL-2 or a biological portion thereof; IL-15 or a biological portion thereof; or IL-21 or a biological portion thereof; or combinations thereof.

19. The engineered NK cell of claim 16, wherein the cytokine is membrane-bound.

20. The engineered NK cell of claim 19, wherein the membrane-bound cytokine is membrane-bound IL-2 (mbIL-2); membrane-bound IL-15 (mbIL-15); membrane-bound IL-21 (mbIL-21); or combinations thereof.

21. The engineered NK cell of any of claims 2-20, wherein the heterologous nucleic acid encoding the immunomodulator is stably integrated into the genome of the cell.

22. The engineered NK cell of any of claims 2-21, wherein the heterologous nucleic acid encoding the immunomodulator is transiently expressed.

23. The engineered NK cell of any one of claims 1-22, wherein the g-NK cell has a surface phenotype that is CD16^{pos}/CD57^{pos}/CD7^{dim/neg}/CD161^{neg}.

24. The engineered NK cell of any of claims 1-23, wherein the g-NK cell further has a surface phenotype that is NKG2A^{neg}/CD161^{neg}.

25. The engineered NK cell of any of claims 1-24, wherein the g-NK cell further is CD38^{neg}.

26. The engineered NK cell of any of claims 1-25, wherein the g-NK cell has a surface phenotype that further is CD45^{pos}/CD3^{neg}/CD56^{pos}.

27. The engineered NK cell of any of claims 1-26, wherein the g-NK cells comprise CD16 158V/V (V158)

28. The engineered NK cell of any of claims 1-26, wherein the g-NK cells are CD16 158V/F.

29. A composition comprising a plurality of engineered g-NK cells of any of claims 1, 3-13, or 23-28.

30. A composition comprising a plurality of engineered g-NK cells of any of claims 2 or 14-28.

31. A composition comprising a plurality of engineered g-NK cells of any of claims 3-28.

32. The composition of any one of claims 29-31, wherein greater than at or about 50% of the NK cells or total cells or greater than at or about 60% of the NK cells or total cells in the composition are g-NK cells.

33. The composition of any one of claims 29-31, wherein greater than at or about 70% of the NK cells or total cells in the composition are g-NK cells.

34. The composition of any one of claims 29-31, wherein greater than at or about 80% of the NK cells or total cells in the composition are g-NK cells.

35. The composition of any one of claims 29-31, wherein greater than at or about 90% of the NK cells or total cells in the composition are g-NK cells.

36. The composition of any one of claims 29-31, wherein greater than at or about 95% of the NK cells or total cells in the composition are g-NK cells.

37. The composition of any one of claims 29 or 31-36, wherein the plurality of engineered g-NK cells comprises greater than at or about 20%, greater than at or about 30%, greater than at or about 40%, greater than at or about 50%, greater than at or about 60%, or greater than at or about 70% g-NK cells comprising a heterologous nucleic acid encoding the CAR.

38. The composition of any one of claims 29 or 31-37, wherein the total composition comprises greater than at or about 20%, greater than at or about 30%, greater than at or about 40%, greater than at or about 50%, greater than at or about 60%, or greater than at or about 70% g-NK cells comprising a heterologous nucleic acid encoding the CAR.

39. The composition of any one of claims 30-36, wherein the plurality of engineered g-NK cells comprises greater than at or about 20%, greater than at or about 30%, greater than at or about 40%, greater than at or about 50%, greater than at or about 60%, or greater than at or about 70% g-NK cells comprising a heterologous nucleic acid encoding the immunomodulator.

40. The composition of any one of claims 30-36 or 39, wherein the total composition comprises greater than at or about 20%, greater than at or about 30%, greater than at or about 40%, greater than at or about 50%, greater than at or about 60%, or greater than at or about 70% g-NK cells comprising a heterologous nucleic acid encoding the immunomodulator.

41. The composition of any one of claims 31-36, wherein the plurality of engineered g-NK cells comprises greater than at or about 20%, greater than at or about 30%, greater than at or about 40%, greater than at or about 50%, greater than at or about 60%, or greater than at or about 70% g-NK cells comprising a heterologous nucleic acid encoding the CAR and a heterologous nucleic acid encoding the immunomodulator.

42. The composition of any one of claims 31-36 or 41 wherein the total composition comprises greater than at or about 20%, greater than at or about 30%, greater than at or about 40%, greater than at or about 50%, greater than at or about 60%, or greater than at or about 70% g-NK cells comprising a heterologous nucleic acid encoding the CAR and the heterologous nucleic acid encoding the immunomodulator.

43. The composition of any one of claims 29-42, wherein greater than at or about 70% of the g-NK cells are positive for perforin and greater than at or about 70% of the g-NK cells are positive for granzyme B.

44. The composition of any one of claims 29-43, wherein greater than at or about 80% of the g-NK cells are positive for perforin and greater than at or about 80% of the g-NK cells are positive for granzyme B.

45. The composition of any one of claims 29-44, wherein greater than at or about 90% of the g-NK cells are positive for perforin and greater than at or about 90% of the g-NK cells are positive for granzyme B.

46. The composition of any one of claims 29-45, wherein greater than at or about 95% of the g-NK cells are positive for perforin and greater than at or about 95% of the g-NK cells are positive for granzyme B.

47. The composition of any of claims 43-46, wherein:
among the cells positive for perforin, the cells express a mean level of perforin as measured by intracellular flow cytometry that is, based on mean fluorescence intensity (MFI), at least at or about two times the mean level of perforin expressed by cells that are FcRγ^{pos}; and/or
among the cells positive for granzyme B, the cells express a mean level of granzyme B as measured by intracellular flow cytometry that is, based on mean fluorescence intensity (MFI), at least at or about two times the mean level of granzyme B expressed by cells that are FcRγ^{pos}.

48. The composition of any of claims 29-47, wherein greater than 10% of the cells in the composition are capable of degranulation against tumor target cells, optionally as measured by CD107a expression, optionally wherein the degranulation is measured in the absence of an antibody against the tumor target cells.

49. The composition of any of claims 29-48, wherein greater than 10% of the cells in the composition are capable of producing interferon-gamma or TNF-alpha against tumor target cells, optionally wherein the interferon-gamma or TNF-alpha is measured in the absence of an antibody against the tumor target cells.

50. The composition of any of claims 29-49, wherein, among the cells in the composition, greater than at or about 15%, greater than at or about 20%, greater than at or about 30%, greater than at or about 40% or greater than at or about 50% produce an effector cytokine in the presence of cells expressing a target antigen (target cells) and an antibody directed against the target antigen (anti-target antibody).

51. The composition of any of claims 29-50, wherein, among the total cells in the composition or among the g-NK cells in the composition, greater than at or about 30% are positive for NKG2C (NKG2C^{pos}) and/or greater than at or about 50% are negative or low for NKG2A (NKG2A^{neg}).

52. The composition of any of claims 29-51, wherein, among the total cells in the composition or among the g-NK cells in the composition, greater than at or about 35% are positive for NKG2C (NKG2C^{pos}) and/or greater than at or about 60% are negative or low for NKG2A (NKG2A^{neg}).

53. The composition of any of claims 29-52, wherein, among the total cells in the composition or among the g-NK cells in the composition, greater than at or about 40% are positive for NKG2C (NKG2C^{pos}) and/or greater than at or about 70% are negative or low for NKG2A (NKG2A^{neg}).

54. The composition of any of claims 29-52, wherein, among the total cells in the composition or among the g-NK cells in the composition, greater than at or about 45% are positive for NKG2C (NKG2C^{pos}) and/or greater than at or about 80% are negative or low for NKG2A (NKG2A^{neg}).

55. The composition of any of claims 29-52, wherein, among the total cells in the composition or among the g-NK cells in the composition, greater than at or about 50% are positive for NKG2C (NKG2C^{pos}) and/or greater than at or about 85% are negative or low for NKG2A (NKG2A^{neg}).

56. The composition of any of claims 29-52, wherein, among the total cells in the composition or among the g-NK cells in the composition, greater than at or about 55% are positive for NKG2C (NKG2C^{pos}) and/or greater than at or about 90% are negative or low for NKG2A (NKG2A^{neg}).

57. The composition of any of claims 29-52, wherein, among the total cells in the composition, or among the g-NK cells in the composition, greater than at or about 60% are positive for NKG2C (NKG2C^{pos}) and/or greater than at or about 95% are negative or low for NKG2A (NKG2A^{neg}).

58. The composition of any of claims 29-57, wherein, among the total cells in the composition or among the g-NK cells in the composition, greater than at or about 50%, greater than at or about 60%, greater than at or about 70%, greater than at or about 80%, or greater than at or about 90% are CD3809.

59. The composition of any of claims 29-57, wherein, among the total cells in the composition or among the g-NK cells in the composition, greater than at or about 50%, greater than at or about 60%, greater than at or about 70%, greater than at or about 80%, or greater than at or about 90% are CD16^{pos}/CD57^{pos}/CD7^{dim/neg}/CD161^{neg}.

60. The composition of any of claims 29-57, wherein, among the total cells in the composition or among the g-NK cells in the composition, greater than at or about 50%, greater than at or about 60%, greater than at or about 70%, greater than at or about 80%, or greater than at or about 90% are NKG2A^{neg}/CD161^{neg}.

61. The composition of any of claims 29-60, wherein the plurality of g-NK cells are CD16 158V/V (V158).

62. The composition of any of claims 29-60, wherein the plurality of g-NK cells are CD16 158V/F.

63. The composition of any of claims 29-62, wherein the composition comprises at least or about at least 10⁸ cells.

64. The composition of any of claims 29-63, wherein the number of g-NK cells in the composition is from at or about 10⁸ to at or about 10¹² cells, from at or about 10⁸ to at or about 10¹¹ cells, from at or about 10⁸ to at or about 10¹⁰ cells, from at or about 10⁸ to at or about 10⁹ cells, from at or about 10⁹ cells to at or about 10¹² cells, from at or about 10⁹ to at or about 10¹¹ cells, from at or about 10⁹ to at or about 10¹⁰ cells, from at or about 10¹⁰ to at or about 10¹² cells, from at or about 10¹⁰ to at or about 10¹¹ cells, or from at or about 10¹¹ to at or about 10¹² cells.

65. The composition of any of claims 29-64, wherein the number of g-NK cells in the composition is or is about 5×10⁸ cells, is or is about 1×10⁹ cells, is or is about 5×10¹⁰ cells, or is or is about 1×10¹⁰ cells.

66. The composition of any of claims 29-65, wherein the volume of the composition is between at or about 50 mL and at or about 500 mL, optionally at or about 200 mL.

67. The composition of any of claims 29-66, wherein the cells in the composition are from a single donor subject that have been expanded from the same biological sample.

68. The composition of any of claims 29-67, wherein the composition is a pharmaceutical composition.

69. The composition of any of claims 29-68, comprising a pharmaceutically acceptable excipient.

70. The composition of any of claims 29-69, wherein the composition is formulated in a serum-free cryopreservation medium comprising a cryoprotectant.

71. The composition of claim 70, wherein the cryoprotectant is DMSO and the cryopreservation medium is 5% to 10% DMSO (v/v).

72. The composition of claim 71, wherein the cryoprotectant is or is about 10% DMSO (v/v).

73. The composition of any of claims 29-72 that is sterile.

74. A sterile bag, comprising the composition of any of claims 29-73.

75. The sterile bag of claim 74, wherein the bag is a cryopreservation-compatible bag.

76. A method of producing a genetically engineered g-NK cell, comprising introducing into an NK cell deficient in expression of FcRγ chain (g-NK cells) a heterologous nucleic acid encoding a chimeric antigen receptor (CAR).

77. A method of producing a genetically engineered g-NK cell, comprising introducing into the g-NK cell a heterologous nucleic acid encoding an immunomodulator, thereby producing a genetically engineered g-NK cell.

78. A method of producing a genetically engineered g-NK cell, comprising:
(a) introducing into an NK cell deficient in expression of FcRγ chain (g-NK cells) a heterologous nucleic acid encoding a chimeric antigen receptor (CAR), and
(b) introducing into the g-NK cell a heterologous nucleic acid encoding an immunomodulator,
thereby producing a genetically engineered g-NK cell, wherein steps (a) and (b) are carried out simultaneously or sequentially in any order.

79. The method of claim 76 or claim 78, wherein the CAR comprises 1) an antigen binding domain; 2) a flexible linker (spacer); 3) a transmembrane region; and 4) an intracellular signaling domain.

80. The method of claim 79, wherein the antigen binding domain is targeted against a tumor antigen.

81. The method of claim 79 or 80, wherein the antigen binding domain is a single chain variable fragment (scFv).

82. The method of any of claims 79-81, wherein the intracellular signaling domain comprises one or more signaling domain from CD3ζ, DAP10, DAP12, CD28, 4-1BB, or OX40.

83. The method of any of claims 79-82, wherein the intracellular signaling domain comprises two or more signaling domains from CD3ζ, DAP10, DAP12, CD28, 4-1BB, or OX40.

84. The method of any of claims 79-83, wherein the intracellular signaling domain comprises a primary signaling domain comprising a signaling domain of CD3ζ and a costimulatory signaling domain.

85. The method of claim 84, wherein the costimulatory signaling domain is a signaling domain of CD28 or 4-1BB.

86. The method of any of claims 76 or 78-85, wherein the heterologous nucleic acid encoding the CAR is introduced under conditions for stable integration into the genome of the g-NK cell.

87. The method of any of claims 76 or 78-86, wherein the heterologous nucleic acid encoding the CAR is comprised in a viral vector and is introduced into the g-NK cell by transduction.

88. The method of claim 87, wherein the viral vector is a lentiviral vector.

89. The method of any one of claims 76 or 78-85, wherein the nucleic acid encoding the CAR is introduced under conditions for transient expression in the g-NK cell.

90. The method of any one of claims 76, 78-85, or 89, wherein the nucleic acid encoding the CAR is introduced by non-viral delivery.

91. The method of any of claims 76, 78-85, or 89-90, wherein the nucleic acid encoding the CAR is introduced to the g-NK cell via a lipid nanoparticle.

92. The method of any of claims 76 or 78-91, wherein the nucleic acid encoding the CAR is DNA.

93. The method of any of claims 76, 78-85, or 89-91, wherein the nucleic acid encoding the CAR is RNA.

94. The method of claim 93, wherein the RNA is mRNA.

95. The method of claim 94, wherein the RNA is a self-amplifying mRNA.

96. The method of any of claims 76, 78-85, or 89-95, wherein the nucleic acid is introduced to the g-NK cell via electroporation.

97. The method of claim 77 or claim 78, wherein the immunomodulator is an immunosuppressant.

98. The method of any one of claim 77 or claim 78 wherein the immunomodulator is an immunoactivator.

99. The method of claim 77, 78, or claim 98, wherein the immunomodulator is a cytokine.

100. The method of claim 99, wherein the cytokine is secretable from the engineered NK cell.

101. The method of claim 100, wherein the secretable cytokine is IL-2 or a biologically active portion thereof; IL-15 or a biologically active portion thereof; IL-21 or a biologically active portion thereof or combinations thereof.

102. The method of claim 99, wherein the cytokine is membrane bound.

103. The method of claim 102, wherein the membrane-bound cytokine is membrane bound IL-2 (mbIL-2); membrane-bound IL-15 (mbIL-15); or membrane bound IL-21 (mbIL-21) or combinations thereof.

104. The method of any one of claims 77-103, wherein the nucleic acid encoding the immunomodulator is introduced under conditions for stable integration into the genome of the g-NK cell.

105. The method of any of claims 77-104, wherein the nucleic acid encoding an immunomodulator is comprised in a viral vector and is introduced into the g-NK cell by transduction.

106. The method of claim 105, wherein the viral vector is a lentiviral vector.

107. The method of any one of claims 77-103, wherein the nucleic acid encoding the immunomodulator is introduced under conditions for transient expression in the g-NK cell.

108. The method of any one of claims 77-85, 89-90, or 97-107, wherein the nucleic acid encoding the immunomodulator is introduced by non-viral delivery.

109. The method of any of claims 77-85, 89-91, or 97-108, wherein the nucleic acid encoding the immunomodulator is introduced to the g-NK cell via a lipid nanoparticle.

110. The method of any of claims 77-109, wherein the nucleic acid encoding the immunomodulator is DNA.

111. The method of any of claims 77-85, 89-91, or 97-109, wherein the nucleic acid encoding the immunomodulator is RNA.

112. The method of claim 111, wherein the RNA is mRNA.

113. The method of claim 112, wherein the RNA is a self-amplifying mRNA.

114. The method of any of claims 77-85, 89-103, or 107-113, wherein the nucleic acid encoding the immunomodulator is introduced to the g-NK cell via electroporation.

115. The method of any of claims 78-114, wherein the nucleic acid encoding the CAR and the nucleic acid encoding the immunomodulator are encoded from the same polynucleotide and introduced together.

116. The method of claim 115, wherein the nucleic acid encoding the CAR and the nucleic acid encoding the immunomodulator are separated by a multicistronic element of the polynucleotide sequence.

117. The method of claim 116, wherein the multicistronic element is a self-cleaving peptide selected from the group consisting of T2A, P2A and F2A.

118. The method of any of claims 78-114, wherein the nucleic acid encoding the CAR and the nucleic acid encoding the immunomodulator are introduced simultaneously during an ex vivo process for expanding a population enriched in g-NK cells.

119. The method of any of claims 76-118, wherein the g-NK cell composition has been produced by ex vivo expansion of NK cells enriched from a biological sample from a subject that are either (i) negative or low for CD3 and positive for CD57 (CD3^{neg}CD57^{pos}) or (ii) negative or low for CD3 and positive for CD56 (CD3^{neg}CD56^{pos}), wherein the enriched NK cells are cultured with irradiated HLA-E+ feeder cells and one or more recombinant cytokines. 76. The method of claim 75, wherein the one or more recombinant cytokine is selected from an effective amount of SCF, GSK3i, FLT3, IL-2, IL-6, IL-7, IL-15, IL-12, IL-18, IL-21, IL-27, or combinations thereof.

120. The method of claim 119, wherein the one or more recombinant cytokine comprises IL-21.

121. The method of claim 119 or claim 120, wherein the culturing is performed in the presence of two or more recombinant cytokines, wherein at least one recombinant cytokine is interleukin (IL)-2 and at least one recombinant cytokine is IL-21.

122. The method of any of claims 76, 79-96, or 119-121, wherein the introducing is carried out during a method for expanding FcRγ-deficient NK (g-NK) cells, said method comprising:
(A) obtaining a population of primary human cells enriched for natural killer (NK) cells, wherein the population enriched for NK cells is selected from a biological sample from a human subject; and
(B) culturing the population of engineered NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21;
wherein the introducing the nucleic acid encoding the CAR is performed after step (A) and prior to, during or after step (B), thereby producing a population of engineered NK cells; and
wherein the method produces an expanded population of engineered NK cells that are enriched in g-NK cells and comprise cells engineered with a CAR.

123. The method of any of claims 76, 79-96, or 119-121, wherein the introducing is carried out during a method for expanding FcRγ-deficient NK cells (g-NK), said method comprising:
(A) obtaining a population of primary human cells enriched for natural killer (NK) cells, wherein the population enriched for NK cells is selected from a biological sample from a human subject;
(B) introducing into the population of enriched NK cells the nucleic acid encoding a CAR, thereby producing a population of engineered NK cells; and
(C) culturing the population of engineered NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21, thereby producing an expanded population of NK cells; and
wherein the method produces an expanded population of engineered NK cells that are enriched in g-NK cells and comprise cells engineered with a CAR.

124. The method of any of claims 76, 79-96, or 119-121, wherein the introducing is carried out during a method for expanding FcRγ-deficient NK cells (g-NK), said method comprising:
(A) obtaining a population of primary human cells enriched for natural killer (NK) cells, wherein the population enriched for NK cells is selected from a biological sample from a human subject; and
(B) culturing the population of enriched NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21, thereby producing an expanded population of NK cells; and
(C) introducing into NK cells of the expanded population of NK cells the nucleic acid encoding a CAR,
wherein the method produces an expanded population of engineered NK cells that are enriched in g-NK cells and comprises cells engineered with a CAR.

125. The method of any of claims 76, 79-96, or 119-121, wherein the introducing is carried out during a method for expanding FcRγ-deficient NK cells (g-NK), said method comprising:
(A) obtaining a population of primary human cells enriched for natural killer (NK) cells, wherein the population enriched for NK cells is selected from a biological sample from a human subject; and
(B) performing a first expansion comprising culturing the population of enriched NK cells in culture medium under conditions for expanding the NK cells to produce a first expanded population of NK cells;
(C) introducing into NK cells of the first expanded population of NK cells the nucleic acid encoding a CAR, thereby producing a population of engineered NK cells; and
(D) performing a second expansion comprising culturing the population of engineered NK cells under conditions for further expansion of the NK cells,
wherein the first expansion and/or second expansion comprise culturing the population of enriched NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21; and
wherein the method produces an expanded population of engineered NK cells that are enriched in g-NK cells and comprise cells engineered with a CAR.

126. The method of any of claims 77, 97-114, or 119-121, wherein the introducing is carried out during a method for expanding FcRγ-deficient NK (g-NK) cells, said method comprising:
(A) obtaining a population of primary human cells enriched for natural killer (NK) cells, wherein the population enriched for NK cells is selected from a biological sample from a human subject; and
(B) culturing the population of engineered NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21;
wherein the introducing the nucleic acid encoding the immunomodulator is performed after step (A) and prior to, during or after step (B), thereby producing a population of engineered NK cells; and
wherein the method produces an expanded population of engineered NK cells that are enriched in g-NK cells and comprises cells engineered with an immunomodulator.

127. The method of any of claims 77, 97-114, or 119-121, wherein the introducing is carried out during a method for expanding FcRγ-deficient NK cells (g-NK), said method comprising:
(A) obtaining a population of primary human cells enriched for natural killer (NK) cells, wherein the population enriched for NK cells is selected from a biological sample from a human subject;
(B) introducing into the population of enriched NK cells the nucleic acid encoding an immunomodulator, thereby producing a population of engineered NK cells; and
(C) culturing the population of engineered NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21, thereby producing an expanded population of NK cells; and
wherein the method produces an expanded population of engineered NK cells that are enriched in g-NK cells and comprise cells engineered with an immunomodulator.

128. The method of any of claims 77, 97-114, or 119-121, wherein the introducing is carried out during a method for expanding FcRγ-deficient NK cells (g-NK), said method comprising:
(A) obtaining a population of primary human cells enriched for natural killer (NK) cells, wherein the population enriched for NK cells is selected from a biological sample from a human subject; and
(B) culturing the population of enriched NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21, thereby producing an expanded population of NK cells; and
(C) introducing into NK cells of the expanded population of NK cells the nucleic acid encoding an immunomodulator,
wherein the method produces an expanded population of engineered NK cells that are enriched in g-NK cells and comprise cells engineered with an immunomodulator.

129. The method of any of claims 77, 97-114, or 119-121, wherein the introducing is carried out during a method for expanding FcRγ-deficient NK cells (g-NK), said method comprising:
(A) obtaining a population of primary human cells enriched for natural killer (NK) cells, wherein the population enriched for NK cells is selected from a biological sample from a human subject; and
(B) performing a first expansion comprising culturing the population of enriched NK cells in culture medium under conditions for expanding the NK cells to produce a first expanded population of NK cells;
(C) introducing into NK cells of the first expanded population of NK cells (the nucleic acid encoding an immunomodulator, thereby producing a population of engineered NK cells; and
(D) performing a second expansion comprising culturing the population of engineered NK cells under conditions for further expansion of the NK cells,
wherein the first expansion and/or second expansion comprise culturing the population of enriched NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21; and
wherein the method produces an expanded population of engineered NK cells that are enriched in g-NK cells and comprise cells engineered with an immunomodulator.

130. The method of any of claims 78-121, wherein the introducing is carried out during a method for expanding FcRγ-deficient NK (g-NK) cells, said method comprising:
(A) obtaining a population of primary human cells enriched for natural killer (NK) cells, wherein the population enriched for NK cells is selected from a biological sample from a human subject; and
(B) culturing the population of engineered NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21;
wherein the introducing (i) the nucleic acid encoding the CAR and/or (ii) the nucleic acid encoding the immunomodulator is performed after step (A) and prior to, during or after step (B), wherein steps (i) and (ii) are carried out simultaneously or sequentially in any order, thereby producing a population of engineered NK cells; and
wherein the method produces an expanded population of engineered NK cells that are enriched in g-NK cells and comprise cells engineered with a CAR and an immunomodulator.

131. The method of any of claims 78-121, wherein the introducing is carried out during a method for expanding FcRγ-deficient NK cells (g-NK), said method comprising:
(A) obtaining a population of primary human cells enriched for natural killer (NK) cells, wherein the population enriched for NK cells is selected from a biological sample from a human subject;
(B) introducing into the population of enriched NK cells (i) the nucleic acid encoding a CAR, and (ii) the nucleic acid encoding an immunomodulator, wherein steps (i) and (ii) are carried out simultaneously or sequentially in any order, thereby producing a population of engineered NK cells; and
(C) culturing the population of engineered NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21, thereby producing an expanded population of NK cells; and
wherein the method produces an expanded population of engineered NK cells that are enriched in g-NK cells and comprise cells engineered with a CAR and an immunomodulator.

132. The method of any of claims 78-121, wherein the introducing is carried out during a method for expanding FcRγ-deficient NK cells (g-NK), said method comprising:
(A) obtaining a population of primary human cells enriched for natural killer (NK) cells, wherein the population enriched for NK cells is selected from a biological sample from a human subject; and
(B) culturing the population of enriched NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21, thereby producing an expanded population of NK cells; and
(C) introducing into NK cells of the expanded population of NK cells (i) the nucleic acid encoding a CAR, and (ii) the nucleic acid encoding an immunomodulator, wherein steps (i) and (ii) are carried out simultaneously or sequentially in any order,
wherein the method produces an expanded population of engineered NK cells that are enriched in g-NK cells and comprise cells engineered with a CAR and an immunomodulator.

133. The method of any of claims 78-121, wherein the introducing is carried out during a method for expanding FcRγ-deficient NK cells (g-NK), said method comprising:
(A) obtaining a population of primary human cells enriched for natural killer (NK) cells, wherein the population enriched for NK cells is selected from a biological sample from a human subject; and
(B) performing a first expansion comprising culturing the population of enriched NK cells in culture medium under conditions for expanding the NK cells to produce a first expanded population of NK cells;
(C) introducing into NK cells of the first expanded population of NK cells (i) the nucleic acid encoding a CAR, and (ii) the nucleic acid encoding an immunomodulator, wherein steps (i) and (ii) are carried out simultaneously or sequentially in any order, thereby producing a population of engineered NK cells; and
(D) performing a second expansion comprising culturing the population of engineered NK cells under conditions for further expansion of the NK cells,
wherein the first expansion and/or second expansion comprise culturing the population of enriched NK cells in culture medium with (1) irradiated HLA-E+ feeder cells, wherein the feeder cells are deficient in HLA class I and HLA class II and wherein the ratio of irradiated HLA-E+ feeder cells to enriched NK cells is from 1:10 to 10:1; and (2) an effective amount of two or more recombinant cytokines for expansion of the NK cells, wherein at least one recombinant cytokine is interleukin (IL)-2 and at least one recombinant cytokine is IL-21; and
wherein the method produces an expanded population of engineered NK cells that are enriched in g-NK cells and comprise cells engineered with a CAR and an immunomodulator.

134. The method of any of claims 119-133, wherein the population of primary human cells enriched for NK cells are obtained by selecting from a biological sample from a human subject cells that are either (i) negative or low for CD3 and positive for CD57 (CD3^{neg}CD57^{pos}) or (ii) negative or low for CD3 and positive for CD56 (CD3^{neg}CD56^{pos}).

135. The method of claim 134, wherein:
the population enriched for NK cells are cells further selected for cells positive for NKG2C (NKG2C^{pos});
the population enriched for NK cells are cells further selected for cells negative or low for NKG2A (NKG2A^{neg}); or
the population enriched for NK cells are cells further selected for cells positive for NKG2C and negative or low for NKG2A (NKG2C^{pos}NKG2A^{neg}).

136. The method of any of claims 119-135, wherein the human subject is a subject in which at least at or about 20% of natural killer (NK) cells in a peripheral blood sample from the subject are positive for NKG2C (NKG2C^{pos}) and at least 70% of NK cells in the peripheral blood sample are negative or low for NKG2A (NKG2A^{neg}).

137. The method of any of claims 119-136, wherein the subject is CMV-seropositive.

138. The method of any of claims 119-137, wherein the percentage of g-NK cells among NK cells in the biological sample from the subject is greater than at or about 5%, greater than at or about 10% or greater than at or about 30%.

139. The method of any of claims 119-138, wherein the percentage of g-NK cells among the population of enriched NK cells is between at or about 20% and at or about 90%, is between at or about 40% and at or about 90% or is between at or about 60% and at or about 90%.

140. The method of any of claims 119-139, wherein the population enriched for NK cells are cells selected from the biological sample that are negative or low for CD3 and positive for CD57 (CD3^{neg}CD57^{pos}).

141. The method of any of claims 119-140, wherein the population enriched for NK cells are cells selected from the biological sample that are negative or low for CD3 and positive for CD56 (CD3^{neg}CD56^{pos}).

142. The method of any of claims 119-141, wherein the two or more recombinant cytokines further comprises an effective amount of SCF, GSK3i, FLT3, IL-6, IL-7, IL-15, IL-12, IL-18, IL-27, or combinations thereof.

143. The method of any of claims 114-142, wherein the recombinant cytokines are IL-21 and IL-2.

144. The method of any of claims 114-142, wherein the recombinant cytokines are IL-21, IL-2, and IL-15.

145. The method of any of claims 114-144, wherein recombinant IL-21 is added to the culture medium during at least a portion of the culturing at a concentration that is from at or about 10 ng/mL to at or about 100 ng/mL.

146. The method of any of claims 114-145, wherein recombinant IL-21 is added to the culture medium during at least a portion of the culturing at a concentration that is at or about 25 ng/mL.

147. The method of any of claims 114-146, wherein recombinant IL-2 is added to the culture medium during at least a portion of the culturing at a concentration that is from at or about 10 IU/mL to at or about 500 IU/mL.

148. The method of any of claims 114-147, wherein recombinant IL-2 is added to the culture medium during at least a portion of the culturing at a concentration that is at or about 100 IU/mL.

149. The method of any of claims 114-148, wherein recombinant IL-2 is added to the culture medium during at least a portion of the culturing at a concentration that is at or about 500 IU/mL.

150. The method of any of claims 114-142 and 144-149, wherein recombinant IL-15 is added to the culture medium during at least a portion of the culturing at a concentration that is from at or about 1 ng/mL to 50 ng/mL.

151. The method of any of claims 114-142 and 144-148, wherein recombinant IL-15 is added to the culture medium during at least a portion of the culturing at a concentration that is at or about 10 ng/mL.

152. The method of any of claims 114-151, wherein the recombinant cytokines are added to the culture medium beginning at or about the initiation of the culturing.

153. The method of any of claims 114-152, wherein the method further comprises exchanging the culture medium one or more times during the culturing, wherein at each exchange of the culture medium, fresh media containing the recombinant cytokines is added.

154. The method of claim 153, wherein the exchanging of the culture medium is carried out every two or three days for the duration of the culturing.

155. The method of claim 153 or claim 154, wherein exchanging the media is performed after a first expansion without media exchange for up to 5 days, optionally after a first expansion without media exchange for up to 5 days.

156. The method of any of claims 114-155, wherein the human subject has the CD16 158V/V NK cell genotype or the CD16 158V/F NK cell genotype, optionally wherein the biological sample is from a human subject selected for the CD16 158V/V NK cell genotype or the CD16 158V/F NK cell genotype.

157. The method of any of claims 114-156, wherein the biological sample is or comprises peripheral blood mononuclear cells (PBMCs), optionally is a blood sample, an apheresis sample or a leukapheresis sample.

158. The method of any of claims 114-157, wherein the HLA-E+ feeder cells are K562 cells transformed with HLA-E (K562-HLA-E).

159. The method of any of claims 114-158, wherein the HLA-E+ feeder cells are 221.AEH cells.

160. The method of any of claims 114-159, wherein the ratio of irradiated HLA-E+ feeder cells to enriched NK cells is between 1:1 and 5:1, inclusive is between 1:1 and 3:1, inclusive, optionally is or is about 2.5:1 or is or is about 2:1 or is about 1:1

161. The method of any of claims 114-160, wherein the recombinant cytokines added to the culture medium during at least a portion of the culturing are 500 IU/mL IL-2, 10 ng/mL IL-15, and 25 ng/mL IL-21.

162. The method of any of claims 114-161, wherein the population of enriched NK cells comprises between at or about 2.0×10⁵ enriched NK cells and at or about 5.0×10⁷ enriched NK cells, between at or about 1.0×10⁶ enriched NK cells and at or about 1.0×10⁸ enriched NK cells, between at or about 1.0×10⁷ enriched NK cells and at or about 5.0×10⁸ enriched NK cells, or between at or about 1.0×10⁷ enriched NK cells and at or about 1.0×10⁸ enriched NK cells, each inclusive, optionally wherein the population of enriched NK cells comprises at or about 1.0×10⁶ enriched NK cells.

163. The method of any of claims 114-162, wherein the population of enriched NK cells at the initiation of the culturing is at a concentration of between or between about 0.05×10⁶ enriched NK cells/mL and 1.0×10⁶ enriched NK cells/mL or between or between about 0.05×10⁶ enriched NK cells/mL and 0.5×10⁶ enriched NK cells/mL, optionally wherein the population of enriched NK cells at the initiation of the culturing comprises a concentration of or about 0.2×10⁶ enriched NK cells/mL.

164. The method of any of claims 114-163, wherein the culturing is carried out until a time at which the method achieves expansion of at least or at least about 2.50×10⁸ g-NK cells, at least or at least about 5.00×10⁸ g-NK cells, at least or at least about 1.0×10⁹ g-NK cells or at least or at least about 5.0×10⁹ g-NK cells.

165. The method of any of claims 114-164, wherein the culturing is carried out for or about or at least or at least about 5 days, 6 days, 7 days, 8 days, 9 days, 10 days, 11 days, 12 days, 13 days, 14 days, 15 days, 16 days, 17 days, 18 days, 19 days, 20 day, 21 days, 22 days, 23 days, 24 days or 25 days.

166. The method of any of claims 114-165 further comprising collecting the expanded population of engineered NK cells produced by the method.

167. The method of any of claims 114-166, further comprising formulating the expanded population of engineered NK cells in a pharmaceutically acceptable excipient.

168. The method of claim 167, further comprising formulating the expanded population of engineered NK cells with a serum-free cryopreservation medium comprising a cryoprotectant.

169. The method of claim 168, wherein the cryoprotectant is DMSO, optionally wherein the cryoprotectant is DMSO and the cryopreservation medium is 5% to 10% DMSO (v/v), optionally is or is about 10% DMSO (v/v).

170. The method of any of claims 114-169, wherein, among the expanded population of engineered NK cells produced by the method, greater than 50% of the population are FcRγ^{neg}, greater than 60% of the population are FcRγ^{neg}, greater than 70% of the population are FcRγ^{neg}, greater than 80% of the population are FcRγ^{neg}, greater than 90% of the population are FcRγ^{neg} or greater than 95% of the population are FcRγ^{neg}.

171. A composition comprising a plurality of engineered NK cells produced by the method of any one of claims 76, 78-96, 119-125, or 134-170-.

172. A composition comprising a plurality of engineered NK cells produced by the method of any one of claims 77, 97-114, 119-121, 126-129, or 134-170.

173. A composition comprising a plurality of engineered NK cells produced by the method of any one of claims 77-121 or 130-170.

174. A method of treating a disease or condition in a subject, comprising administering an effective amount of cells of the composition of any of claims 29-73 or 171-173 to an individual in need thereof.

175. The method of claim 174, wherein the disease or condition is selected from the group consisting of an inflammatory condition, an infection, or cancer.

176. The method of claim 174 or claim 175, wherein the disease or condition is a cancer and the cancer is leukemia, a lymphoma, or a myeloma.

177. The method of claim 174 or claim 175, wherein the disease or condition is a cancer and the cancer comprises a solid tumor.

178. The method of claim 177, wherein the cancer is selected from among an adenocarcinoma of the stomach or gastroesophageal junction, a bladder cancer, a breast cancer, a brain cancer, a cervical cancer, a colorectal cancer, an endocrine/neuroendocrine cancer, a head and neck cancer, a gastrointestinal stromal cancer, a giant cell tumor of the bone, a kidney cancer, a liver cancer, a lung cancer, a neuroblastoma, an ovarian epithelial/fallopian tube/primary peritoneal cancers, a pancreatic cancer, a prostate cancer, a skin cancer, and a soft tissue carcinoma.

179. The method of any of claims 174-178, wherein the composition is administered as a monotherapy.

180. The method of any of claims 174-179, further comprising administering an additional agent to the individual for treating the disease or condition.

181. The method of claim 180, wherein the additional agent is an antibody or an Fc-fusion protein.

182. The method of claim 180 or claim 181, wherein the additional agent is an antibody that is a monoclonal antibody.

183. The method of claim 181 or claim 182, wherein the antibody is a full-length antibody.

184. The method of any of claims 181-183, wherein the antibody is an IgG1 antibody.

185. The method of claim 180-184, wherein the disease or condition is a cancer and the additional agent, optionally the antibody, recognizes a tumor antigen associated with the cancer.

186. The method of any of claims 174-185, comprising administering from at or about 1×10⁵ to at or about 50×10⁹ cells of the g-NK cell composition to the individual.

187. The method of any of claims 174-186, comprising administering from at or about from at or about 1×10⁸ cells to at or about 50×10⁹ NK cells of the g-NK cell composition, optionally at or about 5×10⁸ cells of the g-NK cell composition, at or about 5×10⁹ cells of the g-NK cell composition or at or about 10×10⁹ cells of the g-NK cell composition.

188. The method of any one of claims 174-187, further comprising administering exogenous cytokine support to facilitate expansion or persistence of the administered NK cells in vivo in the subject, optionally wherein the exogenous cytokine is or comprises IL-15.

189. The method of any of claims 174-188, wherein prior to the administration of the dose of g-NK cells, the subject has received a lymphodepleting therapy.

190. The method of claim 129, wherein the lymphodepleting therapy comprises fludarabine and/or cyclophosphamide.

191. The method of claim 189 or claim 190, wherein the lymphodepleting comprises the administration of fludarabine at or about 20-40 mg/m² body surface area of the subject, optionally at or about 30 mg/m², daily, for 2-4 days, and/or cyclophosphamide at or about 200-400 mg/m² body surface area of the subject, optionally at or about 300 mg/m², daily, for 2-4 days.

192. The method of any of claims 189-191, wherein the lymphodepleting therapy comprises fludarabine and cyclophosphamide.

193. The method of any of claims 189-192, wherein the lymphodepleting therapy comprises the administration of fludarabine at or about 30 mg/m² body surface area of the subject, daily, and cyclophosphamide at or about 300 mg/m² body surface area of the subject, daily, each for 2-4 days, optionally 3 days.

194. The method of any of claims 189-193, wherein administration of the cells is initiated within two weeks or at or about two weeks after initiation of the lymphodepleting therapy.

195. The method of any one of claims 174-194, wherein the individual is a human.

196. The method of any one of claims 174-195, wherein the NK cells in the composition are allogenic to the individual.

197. The method of any one of claims 189-195, wherein the NK cells in the composition are autologous to the subject.
